(19) United States
(12) Patent Application Publication
(10) Pub. No.: US 2026/0043085 A1
(43) Pub. Date: Feb. 12, 2026

(54) COMPOSITIONS, METHODS AND KITS FOR DIAGNOSIS OF A GASTROENTEROPANCREATIC NEUROENDOCRINE NEOPLASM

(71) Applicant: Clifton Life Sciences LLC, Charlestown (KN)

(72) Inventors: Irvin Mark MODLIN, Woodbridge, CT (US); Mark KIDD, New Haven, CT (US); Ignat DROZDOV, Stratford Upon Avon (GB)

(21) Appl. No.: 19/071,035

(22) Filed: Mar. 5, 2025

Related U.S. Application Data
(63) Continuation of application No. 17/521,205, filed on Nov. 8, 2021, now Pat. No. 12,258,633.
Application No. 17/521,205 is a continuation of application No. 16/528,864, filed on Aug. 1, 2019, now Pat. No. 11,168,372.
Application No. 16/528,864 is a division of application No. 14/855,229, filed on Sep. 15, 2015, now Pat. No. 10,407,730.
(60) Provisional application No. 62/050,465, filed on Sep. 15, 2014.

(51) Int. Cl. C12Q 1/6886; G01N 33/574

(52) U.S. Cl. CPC C12Q 1/6886; G01N 33/57407; C12Q 2600/112; C12Q 2600/118; C12Q 2600/158

(57) ABSTRACT

Methods are provided for diagnosing, detecting, or prognosticating a GEP-NEN based on the expression level score of biomarkers exhibiting differential expression in subjects having a GEP-NEN relative to a reference or control sample. The invention also provides compositions and kits comprising these biomarkers and methods of using these biomarkers in subsets or panels thereof to diagnose, classify, and monitor GEP-NEN and types of GEP-NEN. The methods and compositions provided herein may be used to diagnose or classify a subject as having a GEP-NEN, to distinguish between different stages of GEP-NENs, e.g., stable or progressive, to provide a measure of risk of developing a progressive GEP-NEN, and to gauge the completeness of treatments for GEP-NEN including, but not limited to surgery and somatostatin therapy.

### CROSS REFERENCE TO RELATED APPLICATIONS

This application is a Continuation of U.S. patent application Ser. No. 17/521,205, filed on Nov. 8, 2021, now U.S. Pat. No. 12,258,633, which is a Continuation of U.S. patent application Ser. No. 16/528,864, filed on Aug. 1, 2019, now U.S. Pat. No. 11,168,372. U.S. patent application Ser. No. 16/528,864 is a Divisional of U.S. patent application Ser. No. 14/855,229, filed on Sep. 15, 2015, now U.S. Pat. No. 10,407,730. U.S. patent application Ser. No. 14/855,229 claims the priority to, and the benefit of, U.S. Provisional Application Ser. No. 62/050,465, filed on Sep. 15, 2014. The contents of each of the aforementioned applications are incorporated by reference in their entireties.

### SEQUENCE LISTING

The Sequence Listing XML associated with this application is provided electronically in XML format and is hereby incorporated by reference into the specification. The name of the XML file containing the Sequence Listing XML is “CLSL-001_C03US_SeqList.xml”. The XML file is 270,805 bytes, created on Mar. 5, 2025, and is being submitted electronically via USPTO Patent Center.

### BACKGROUND OF THE INVENTION

Gastroenteropancreatic (GEP) neuroendocrine neoplasm (GEP-NEN), also referred to as Gastroenteropancreatic Neuroendocrine Tumor and Neuroendocrine Tumor (NET), is the second most prevalent malignant tumor in the gastrointestinal (GI) tract in the U.S. Incidence and prevalence have increased between 100 and 600 percent in the U.S. over the last thirty years, with no significant increase in survival.

Heterogeneity and complexity of GEP-NENs has made diagnosis, treatment, and classification difficult. These neoplasms lack several mutations commonly associated with other cancers and microsatellite instability is largely absent. See Tannapfel A, Vomschloss S, Karhoff D, et al., “BRAF gene mutations are rare events in gastroenteropancreatic neuroendocrine tumors,” Am J Clin Pathol 2005; 123 (2): 256-60; Banck M, Kanwar R, Kulkarni A A, et al., “The genomic landscape of small intestine neuroendocrine tumors,” J Clin Invest 2013; 123 (6): 2502-8; Zikusoka M N, Kidd M, Eick G, et al., Molecular genetics of gastroenteropancreatic neuroendocrine tumors. Cancer 2005; 104:2292-309; Kidd M, Eick G, Shapiro M D, et al. Microsatellite instability and gene mutations in transforming growth factor-beta type II receptor are absent in small bowel carcinoid tumors,” Cancer 2005; 103 (2): 229-36.

Individual histopathologic subtypes as determined from tissue resources e.g., biopsy, associate with distinct clinical behavior, yet there is no definitive, generally accepted pathologic classification or prediction scheme, hindering treatment assessment and follow-up.

Existing diagnostic and prognostic approaches for GEP-NENs include imaging (e.g., CT or MRI), histology, measurements of circulating hormones and proteins associated with NENs e.g., chromogranin A and detection of some gene products. Available methods are limited, for example, by low sensitivity and/or specificity, inability to detect early-stage disease, or exposure to radiation risk. GEP-NENs often go undiagnosed until they are metastatic and often untreatable. In addition, follow-up is difficult, particularly in patients with residual disease burden.

There is a need for specific and sensitive methods and agents for the detection of GEP-NEN, including stable and progressive GEP-NEN, for example, for use in diagnosis, prognosis, prediction, staging, classification, treatment, monitoring, and risk assessment, and for investigating and understanding molecular factors of pathogenesis, malignancy, and aggressiveness of this disease. For example, such methods and agents are needed that can be repeatedly and directly collected with low risk exposure e.g., non-invasive peripheral blood test, be performed simply, rapidly, and at relatively low cost.

The present application overcomes the above-noted problems by providing novel compositions, methods, and kits for accurately diagnosing, detecting, and monitoring the presence of GEP-NENs and/or the types or stage of GEP-NEN in circulating peripheral blood samples. The described embodiments furthermore may be used to identify a level of risk for a patient to develop a progressive GEP-NEN, and/or to determine the risk of residual or reoccurring progressive GEP-NEN in a post-surgery or post-somatostatin treated human patient. In addition, it can be used as a prognostic for predicting response to therapy e.g., peptide receptor radiotherapy (PRRT).

### SUMMARY OF THE INVENTION

In one aspect, the present invention relates to gastroenteropancreatic neuroendocrine neoplasm (GEP-NEN) biomarkers measured in circulating blood, the detection of which may be used in diagnostic, prognostic and predictive methods. Among the provided objects are GEP-NEN biomarkers, feature subsets and panels of the biomarkers, agents for binding and detecting the biomarkers, kits and systems containing such agents, and methods and compositions for detecting the biomarkers, for example, in biological samples e.g., blood, as well as prognostic, predictive, diagnostic, and therapeutic uses thereof.

Provided are agents, sets of agents, and systems containing the agents for GEP-NEN prognosis, detection and diagnosis. Typically, the systems include a plurality of agents (e.g., set of agents), where the plurality specifically binds to and/or detects a plurality of GEP-NEN biomarkers in a panel of GEP-NEN biomarkers. The agents may be isolated polypeptides or polynucleotides which specifically bind to one or more GEP-NEN biomarkers. For example, provided are sets of isolated polynucleotides and polypeptides that bind to a panel of GEP-NEN biomarkers, and methods and uses of the same.

Also provided are prognostic, diagnostic, and predictive methods and uses of the agents, compositions, systems, and kits for GEP-NEN and associated conditions, syndromes and symptoms. For example, provided are methods and uses for detection, diagnosis, classification, prediction, therapeutic monitoring, prognosis, or other evaluation of GEP-NEN or an outcome, stage or level of aggressiveness or risk thereof, or associated condition. In some embodiments, the methods are performed by determining the presence, absence, expression levels, or expression profile of a GEP-NEN biomarker, more typically a plurality of GEP-NEN biomarkers, such as a feature subset chosen from a panel of biomarkers, and/or comparing such information with normal or reference expression levels or profiles or standards. Thus, in some embodiments, the methods are carried out by obtaining a biological test sample and detecting the presence, absence, expression level score, or expression profile of a GEP-NEN biomarker as described herein. For example, the methods can be performed with any of the systems of agents, e.g., polynucleotides or polypeptides, provided herein. For example, the methods generally are carried out using one or more of the provided systems.

Provided are methods, agents and compositions for detection of and distinguishing between a number of different GEP-NEN types or stages. Exemplary GEP-NEN types and stages include stable disease (SD) and progressive (highly active) disease (PD).

In one aspect, the provided methods and compositions may be used to specifically and sensitively detect different stages of GEP-NENs, such as GEP-NENs in a stable disease (SD) or progressive disease (PD) states; in some aspects, the methods and compositions may be used to predict disease progression, treatment response, and metastasis. Methods and compositions provided herein are useful for diagnosis, prognosis, prediction, staging, classification, treatment, monitoring, assessing risk, and investigating molecular factors associated with GEP-NEN disease.

Provided are such methods capable of being carried out quickly, simply, and at relatively low cost, as compared to other diagnostic and prognostic methods.

Provided are methods and compositions that are useful for defining gene expression-based classification of GEP-NENs, and thus are useful for allowing the prediction of malignancy and metastasis, such as in early stage disease or using histologically negative samples, providing accurate staging, facilitating rational therapy, and in developing large validated clinical datasets for GEP-NEN-specific therapeutics.

The GEP-NEN biomarkers may include a subset of biomarkers, the expression of which is different in or is associated with the presence or absence of GEP-NEN, or is different in or is associated with a particular classification, stage, aggressiveness, severity, degree, metastasis, symptom, risk, treatment responsiveness or efficacy, or associated syndrome. The subset of GEP-NEN biomarkers typically includes at least 22 GEP-NEN biomarkers. In some embodiments, the subset of biomarkers includes at least 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51 GEP-NEN biomarkers, or includes at or about 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51 GEP-NEN biomarkers.

For example, in some aspects, the subset of biomarkers includes at least 22, or at least 38, or at least 51 biomarkers. In a particular example, the subset contains at least 22 biomarkers, or about 22 biomarkers, or 22 biomarkers, chosen from a panel of 38 biomarkers. In some embodiments, the subset of biomarkers includes at least 22, 23, 24, 25, 26, 27, 28, 29, 30, 31, 32, 33, 34, 35, 36, 37, or 38 biomarkers chosen from a panel of 38 biomarkers.

Because the systems, methods, and kits contain a plurality of agents that specifically bind to or hybridize to the biomarkers in the panel, the number of biomarkers generally relates to the number of agents in a particular system. For example, among the provided methods is a method that contains at least 22 binding agents, which specifically hybridizes to or binds to a subset of at least 22 GEP-NEN biomarkers, respectively.

In some aspects, the subset of biomarkers includes at least 22, 23, 24, 25, 26, 27, 28, 29, 30, 31, 32, 33, 34, 35, 36, 37, and/or all of the following group of gene products, including polynucleotides (e.g. 38 transcripts) and polypeptides: PNMA2, NAP1L1, FZD7, SLC18A2/VMAT2, NOL3, SSTR5, TPH1, RAF1, RSF1, SSTR3, SSTR1, CD59, ARAF, APLP2, KRAS, MORF4L2, TRMT112, MKI67/KI67, SSTR4, CTGF, SPATA7, ZFHX3, PHF21A, SLC18A1/VMAT1, ZZZ3, TECPR2, ATP6V1H, OAZ2, PANK2, PLD3, PQBP1, RNF41, SMARCD3, BNIP3L, WDFY3, COMMD9, BRAF, and/or GLT8D1 gene products.

In a particular example, the subset of 22 biomarkers includes PNMA2, NAPILI, FZD7, SLC18A2, NOL3, SSTR5, TPH1, RAF1, RSF1, SSTR3, SSTR1, CD59, ARAF, APLP2, KRAS, MORF4L2, TRMT112, MKI67, SSTR4, CTGF, SPATA7, and ZFHX3 gene products.

Among the provided methods, agents, and systems are those that are able to classify or detect a GEP-NEN in a human blood sample. In some embodiments, the provided systems and methods can identify or classify a GEP-NEN in a human blood sample. In some examples, the systems can provide such information with a specificity, sensitivity, and/or accuracy of at least 75%, 80%, 85%, 90%, 91%, 92%, 93%, 94%, 95%, 96%, 97%, 98%, 99% or 100%, e.g., at least 80%.

In some embodiments, the system can predict treatment responsiveness to, or determine whether a patient has become clinically stable following, or is responsive or non-responsive to, a GEP-NEN treatment, such as a surgical intervention or drug therapy (for example, somatostatin analog therapy). In some cases, the methods and systems do so with a specificity, sensitivity, and/or accuracy of at least 75%, 80%, 85%, 90%, 91%, 92%, 93%, 94%, 95%, 96%, 97%, 98%, 99% or 100%, e.g., with at least 90% accuracy. In some cases, it can differentiate between treated and untreated GEP-NEN with a specificity, sensitivity, and/or accuracy of at least 75%, 80%, 85%, 90%, 91%, 92%, 93%, 94%, 95%, 96%, 97%, 98%, 99% or 100%, e.g., with a sensitivity and specificity of at least 85%.

In some cases, the system can determine diagnostic or prognostic information regarding a subject previously diagnosed with GEP-NEN, for example, whether the subject has a stable disease (SD) or progressive disease (PD) state of GEP-NEN, or is in complete remission, for example, would be clinically categorized as having stable disease, progressive disease, or being in complete remission.

In some embodiments, the agents for detecting the biomarkers, e.g., the sets of polynucleotide or polypeptide agents, and uses thereof, are capable of distinguishing between the presence and absence of GEP-NEN in a biological sample, between GEP-NEN and mucosal samples and GEP-NEN samples, and/or between specific classes or subtypes of GEP-NENs, for example, between aggressive (high activity) and benign (low activity) GEP-NEN samples,

In one aspect, the system is able to classify or detect a GEP-NEN in a human blood sample or human saliva sample. In one aspect, the human sample is whole blood or nucleic acid or protein prepared from whole blood, without first sorting or enriching for any particular population of cells. In one aspect, the system includes agents that bind to biomarkers in a subset of at least 22 GEP-NEN biomarkers.

In some embodiments, in addition to the agents that bind the GEP-NEN biomarkers, the provided systems contain one or more agents that bind to gene products for use in normalization or as controls, for example, housekeeping gene products include ALG9 gene products;

In some embodiments, the methods include selecting a subset of at least 22 biomarkers chosen from a panel of 38 biomarkers useful in generating a classifier for GEP-NEN and different stages of GEP-NEN.

In some embodiments, the methods further include contacting a test sample from the human patient with a plurality of agents specific to the biomarkers in the subset.

The biological test sample used with the methods can be any biological sample, such as tissue, biological fluid, or other sample, including blood samples, such as plasma, serum, whole blood, buffy coat, or other blood sample, tissue, saliva, serum, urine, or semen sample. In some aspects, the sample is obtained from blood. Often, the test sample is taken from a GEP-NEN patient.

The agents can be any agents for detection of biomarkers, and typically are isolated polynucleotides or isolated polypeptides or proteins, such as antibodies, for example, those that specifically hybridize to or bind to a subset or panel of GEP-NEN biomarkers including at least 22 GEP-NEN biomarkers.

In some embodiments, the methods are performed by contacting the test sample with one of the provided agents, more typically with a plurality of the provided agents, for example, one of the provided systems, such as a set of polynucleotides that specifically bind to the subset of GEP-NEN biomarkers. In some embodiments, the set of polynucleotides includes DNA, RNA, cDNA, PNA, genomic DNA, or synthetic oligonucleotides. In some embodiments, the methods include the step of isolating RNA from the test sample prior to detection, such as by RT-PCR, e.g., QPCR. Thus, in some embodiments, detection of the GEP-NEN biomarkers, such as expression levels thereof, includes detecting the presence, absence, or amount of RNA. In one example, the RNA is detected by PCR or by hybridization.

In one aspect, the polynucleotides include sense and antisense primers, such as a pair of primers that is specific to each of the GEP-NEN biomarkers in the subset of biomarkers. In one aspect of this embodiment, the detection of the GEP-NEN biomarkers is carried out by PCR, typically quantitative or real-time PCR. For example, in one aspect, detection is carried out by producing cDNA from the test sample by reverse transcription; then amplifying the cDNA using the pairs of sense and antisense primers that specifically hybridize to the panel of GEP-NEN biomarkers, and detecting products of the amplification. In some embodiments, the GEP-NEN biomarkers include mRNA, cDNA, or protein.

In some embodiments, the methods further include determining a mathematically-derived expression level score of biomarkers selected in the subset in the test sample. This is the MAARC-NET score (Multi-Analyte Risk Classification for NETs). It has two scales 0-8 and the percentage-derivatives scaled to 100% i.e., 0-100%.

The mathematically-derived MAARC-NET score is the product of a classifier built from predictive classification algorithms, e.g. support vector machines (SVM), linear discriminant analysis (LDA), K-nearest neighbor (KNN) and/or naive Bayes (NB). In some examples, the classifier is generated from a combination of SVM, LDA, KNN, and NB classification algorithms and a 10-fold cross-validation design.

In some embodiments, the methods further include a step of determining a mathematically-derived expression level score of biomarkers in the subset in a normal or reference sample, typically carried out prior to the normalization and comparing steps.

The normal or reference sample may be from a healthy patient or a patient who has GEP-NEN. Where the test sample is from a patient with GEP-NEN, the normal or reference sample or level may be from the same or a different patient. For example, the normal or reference sample may be from the GEP-NEN patient from a tissue, fluid or cell not expected to contain GEP-NEN or GEP-NEN cells. On another aspect, the normal or control sample is from the GEP-NEN patient before or after therapeutic intervention, such as after surgery or chemical intervention. In another aspect, the reference or normal sample is from a tissue or fluid that corresponds to the GEP-NEN or metastasis of the test sample, from a healthy individual, such as normal enterochromaffin cell (EC) preparation or small intestinal (SI) sample, or normal liver, lung, bone, blood, saliva, or other bodily fluid, tissue, or biological sample. In another embodiment, the test sample is from a metastasis, plasma, or whole blood or other fluid of a GEP-NEN patient and the reference sample is from primary tumor or fluorescent activated cell (FAC)-sorted tumor cells.

In other aspects, the test sample is from blood and the test biological sample is from the GEP-NEN patient after treatment and the reference sample is from the same GEP-NEN patient as the test biological sample, prior to treatment; the reference sample is from a tissue or fluid not containing GEP-NEN cells; the reference sample is from a healthy individual; the reference sample is from a cancer other than GEP-NEN; the reference sample is from an EC cell or SI tissue; the test biological sample is from a metastatic GEP-NEN and the reference sample is from a non-metastatic GEP-NEN; or the reference sample is from a GEP-NEN of a different classification compared to the GEP-NEN patient from which the test biological sample is obtained.

In one aspect, the test biological sample is from a GEP-NEN patient prior to treatment and the normal or reference sample is from the GEP-NEN patient after treatment. In another aspect, the normal or reference sample is from a non-metastatic tissue of the GEP-NEN patient.

In some cases, a normalization step is performed to normalize the level of expression score of the biomarkers in the subset in the test sample to the level of expression score of the biomarkers in the subset in the reference sample.

In some cases, a comparison step is performed to determine whether there is a difference, such as a significant difference, between the normalized expression level score and a predetermined cut-off value or score threshold. Certain predetermined cut-off values or score thresholds are indicative of different stages of GEP-NEN, while others are indicative of different levels of risk, i.e. low, intermediate, or high, for developing a progressive GEP-NEN.

In one aspect, the methods include comparing the normalized expression level score with a predetermined cutoff value chosen to exclude a control or reference sample, wherein a normalized expression level above the predetermined cutoff value is indicative of a GEP-NEN, wherein the cutoff value is about 2 (on a scale of 0-8, or 13.4% on a scale of 0-100%).

In another aspect, the methods include comparing the normalized expression level score with a predetermined cutoff value chosen to exclude a non-progressive GEP-NEN, wherein a normalized expression level above the predetermined cutoff value of 5 (on a scale of 0-8, or 43.4% on a scale of 0-100%) is indicative of progressive GEP-NEN.

In another aspect, the methods further include identifying the level of risk for a human patient to develop progressive GEP-NEN, wherein a normalized expression level score below about 5 (or 43.4%) is indicative of a low level of risk for developing a progressive GEP-NEN, a normalized expression level score between about 5 and 7 (43.4%-63.4%) is indicative of an intermediate level of risk for developing progressive GEP-NEN, and a normalized expression level score between about 7 and 8 (>63.4%) is indicative of a high level of risk for developing progressive GEP-NEN.

In some cases, a subsequent determination is performed for the actual expression level (not mathematically-derived expression level score) of individual genes, where identifying the intermediate level of risk for developing progressive GEP-NEN further includes determining a first state of intermediate risk, wherein the normalized expression level score between a non-progressive reference sample and the test sample is about 5 (43.4%), the normalized expression level of SMARCD3 is below a first threshold value, and the expression level of TPH1 is below a second threshold value.

In other cases, identifying the intermediate level of risk for developing progressive GEP-NEN further includes determining a second state of intermediate risk, wherein the normalized expression level score between a non-progressive reference sample and the test sample is about 6 (52.7%), the normalized expression level of VMAT1 is equal to or above 0, and the expression level of PHF21A is equal to or above a first threshold value.

In some cases, identifying the intermediate level of risk for developing progressive GEP-NEN further includes determining a third state of intermediate risk, wherein the normalized expression level score between a non-progressive reference sample and the test sample is about 7 (63.4%), the expression level of VMAT1 is equal to or above 0, and the expression level of PHF21A is equal to or below a first threshold value.

In other cases, identifying the high level of risk for developing progressive GEP-NEN further includes determining the normalized expression level score of ZZZ3, wherein the expression level score of ZZZ3 is equal to or less than 14.

Also provided are methods and uses of the provided biomarkers, agents, systems and detection methods for use in determining the risk of residual or reoccurring progressive GEP-NEN in a post-surgery human patient. In such cases, the level of risk for residual or reoccurring progressive GEP-NEN in the post-surgical test sample is identified, wherein a normalized expression level score below about 5 (43.4%) is indicative of a low level of risk, a normalized expression level score between about 5 and 7 (43.4-63.4%) is indicative of an intermediate level of risk, and a normalized expression level score between about 7 and 8>63.4%) is indicative of a high level of risk.

In some cases, identifying the level of risk for residual or reoccurring progressive GEP-NEN further includes determining an elevated expression level score of gene products in at least one gene cluster as determined between a pre-surgical test sample from the patient and the post-surgical test sample.

In some embodiments, the at least one gene cluster includes the proliferome, signalome, secretome I and II, plurome, epigenome, plurome, SSTRome, and combinations thereof.

In other embodiments, the at least one gene cluster includes the PD cluster, the ND cluster, the TD cluster, and the ID cluster. The PD cluster includes the proliferome, signalome, secretome II, plurome, and epigenome. The ND cluster includes the ARAF1, BRAF, KRAS, RAF1, Ki67, NAPILI, NOL3, GLT8D1, PLD3, PNMA2, VMAT2, TPH1, FZD7, MORF4L2, and ZFHX3. The TD cluster includes the Secretome (I), the Plurome, and the SSTRome. The ID cluster includes the Proliferome, secretome (II), plurome, and epigenome.

In other embodiments, determining the elevated expression of gene products in at least one gene cluster includes evaluating a plurality of gene cluster algorithms including the PDA, NDA, TDA, and IDA algorithms.

In some embodiments, the methods further include treating the patient based on the indication of intermediate or high level of risk for residual or recurring progressive GEP-NEN by one of surgery or therapy.

Also provided are methods and uses of the provided biomarkers, agents, systems and detection methods for use in determining the risk of residual or reoccurring progressive GEP-NEN in a post-somatostatin analog treated patient. In such cases, the level of risk for somatostatin treatment failure is identified, wherein a normalized expression level score below about 5 (43.4%) is indicative of a low level of risk, a normalized expression level score between about 5 and 7 (43.4-63.4%) is indicative of an intermediate level of risk, and a normalized expression level score between about 7 and 8 (>63.4%) is indicative of a high level of risk.

The methods may further include determining the difference in expression level score in at least one of the SSTRome and Proliferome gene clusters between a pre-therapy test sample from the human patient and the post-therapy test sample, wherein an increased level of expression score is indicative of increased risk for residual or reoccurring progressive GEP-NEN.

In some cases, a somatostatin analog is administered to the human patient-based on the indication of intermediate or high level of risk for residual or recurring progressive GEP-NEN and an increased level of expression in at least one of the SSTRome and Proliferome gene clusters.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents, and other references mentioned herein are incorporated by reference. The references cited herein are not admitted to be prior art to the claimed invention. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIGS. 1A-1E are graphs showing differential exon expression in a marker gene panel inferred from an Affymetrix Human Exon 1.0 ST array in neuroendocrine tumor (NET) tissue relative to normal intestinal mucosa controls. RMA-normalized exon expressions of (FIG. 1A) Tph1, (FIG. 1B) VMAT2, (FIG. 1C) SCG5, (FIG. 1D) CgA, and (FIG. 1E) PTPRN2 were visualized in normal (green) and tumor (samples).

FIGS. 2A-2E are graphs showing the validation of alternative splicing in marker genes by Reverse transcriptase polymerase chain reaction (RT-PCR). Marker genes (FIG. 2A) Tph1, (FIG. 2B) VMAT2, (FIG. 2C) SCG5, (FIG. 2D) CgA, and (FIG. 2E) PTPRN2 were differentially expressed in NET samples relative to normal mucosa controls.

FIG. 3 is a line graph showing the prediction accuracy of four classification algorithms (SVM, LDA, KNN, and Bayes) using sequential addition of up to 22 significantly up-regulated genes (p<0.05) in GEP-NET samples obtained using the results of 10-fold cross validation.

FIGS. 4A-4C are graphs showing mathematically-derived MAARC-NET scores in the test set. (FIG. 4A) Frequency distribution for the 0-4 score in the controls, SD and PD; (FIG. 4B) Frequency distribution using a 0-8 score in the same sample set; (FIG. 4C) Correlation assessment for each of the two scores in (FIG. 4A) and (FIG. 4B).

FIGS. 5A-5B are graphs showing MAARC-NET scores in the test set and a Receiver Operating Characteristics (ROC) analysis. In FIG. 5A, NETs had a significantly elevated score compared to controls, where values for PD were higher than SD. In FIG. 5B, a ROC curve of controls versus GEP-NETS is shown, wherein the AUC was >0.98, p<0.0001. *p<0.05 vs. controls, #p<0.05 vs. SD (2-tailed Mann-Whitney U-test).

FIG. 6A-6B are (FIG. 6A) a graph of MAARC-NET scores in the independent set, wherein Progressive Disease (PD) NETs had a significantly higher elevated score compared to Stable Disease (SD); and (FIG. 6B) a frequency distribution graph for the 0-8 score in SD and PD. #p<0.0001 vs. SD (2-tailed Mann-Whitney U-test).

FIGS. 7A-7B are (FIG. 7A) a graph of ROC of SD versus PD NETs with an AUC of >0.93, p<0.0001 and (FIG. 7B) a graph of the percentage of SD and PD NETs correctly called using a cut-off score of ≥7.

FIG. 8 is a nomogram for NETest 1 demonstrating how the score is achieved and categorizing patients into different disease classes.

FIG. 9 is a graph of the utility of the nomogram of FIG. 8. The percentages of correctly predicted SD and PD NETs, including the level of disease activity, using the nomogram of FIG. 8 are shown.

FIGS. 10A-10B are graphs each showing the frequency distribution for the 0-8 score in SD and PD NET tumors in (FIG. 10A) the test set and (FIG. 10B) the independent set.

FIGS. 11A-11B are graphs of (FIG. 11A) the frequency distribution for the 0-8 score in SD and PD in the combined sets and (FIG. 11B) the risk probability for a score being either SD or PD vs. NETest Score.

FIG. 12 is a nomogram of NETest 2a with the inclusion of score and risk categorizations. Top figure includes MAARC-NET as 0-8 scores; bottom figure is the 0-100% scaled version.

FIG. 13 is a nomogram of NETest 2 with the inclusion of risk category delineation.

FIGS. 14A-14B are illustrations representing the Hallmarks of Neoplasia refocused on NETs. FIG. 14A shows a delineation of tumor (adenocarcinoma) derived hallmarks from Hanahan D, Weinberg R A: Hallmarks of cancer: the next generation. Cell 2011, 144 (5): 646-674. FIG. 14B shows NET hallmark based on the Hanahan and Weinberg classification.

FIGS. 15A-15B are graphs showing normalized gene expression of gene clusters in (FIG. 15A) normal mucosa and (FIG. 15B) NETs.

FIG. 16 is a graph of normalized gene expression as evaluated by the PDA and NDA algorithms in normal mucosa (NM) and NET.

FIGS. 17A-17C are graphs of normalized gene expression in (FIG. 17A) normal mucosa, (FIG. 17B) a SD related gene cluster, and (FIG. 17C) a PD related gene cluster.

FIG. 18 is a graph of normalized gene expression in an independent test set, where the genes in PD and SD tumors were evaluated.

FIGS. 19A-19B are graphs showing normalized gene expression of PDA and NDA gene cluster algorithms in (FIG. 19A) the test set and (FIG. 19B) the independent set.

FIGS. 20A-20B are graphs showing (FIG. 20A) normalized gene expression of PDA and NDA gene cluster algorithms in the combined set, and (FIG. 20B) a ROC analysis curve of PDA and NDA for differentiating SD from PD, where *p<0.05 vs. SD.

FIGS. 21A-21B are graphs showing normalized gene expression as evaluated by TDA and IDA gene cluster algorithms in (FIG. 21A) the test set, and (FIG. 21B) the independent set.

FIGS. 22A-22B are graphs showing a ROC analysis of (FIG. 22A) TDA and IDA for differentiating SD from PD, and (FIG. 22B) for each of the individual gene clusters.

FIGS. 23A-23B are graphs showing the alternation in (FIG. 23A) NETest Score in Pre- and Post-Surgery conditions and (FIG. 23B) circulating Chromogranin A (CgA) levels in Pre- and Post-Surgery conditions.

FIGS. 24A-24B are illustrations showing differences in the NETest nomogram in (FIG. 24A) pre-surgical therapy conditions and (FIG. 24B) post-surgical therapy conditions.

FIGS. 25A-25B are graphs showing the percentage change in (FIG. 25A) mathematically-derived score and (FIG. 25B) Chromogranin A in both R0 (complete resections) and R1/2 (incomplete sections) conditions.

FIGS. 26A-26D are graphs showing the difference in NETest score for gene-derived algorithms, (FIG. 26A) PDA, (FIG. 26B) NDA, (FIG. 26C) TDA, and (FIG. 26D) IDA, in pre- and post-surgery conditions.

FIGS. 27A-27I are graphs showing the differences in NETest score for gene-derived clusters, (FIG. 27A) SSTRome, (FIG. 27B) Proliferome, (FIG. 27C) Signalome, (FIG. 27D) Metabolome, (FIG. 27E) Secretome, (FIG. 27F) Secretome, (FIG. 27G) Plurome, (FIG. 27H) EpiGenome, and (FIG. 27I) ApopTome, in pre- and post-surgery conditions.

FIG. 28 is a nomogram of NETest 3 with the inclusion of surgically-relevant algorithms and gene clusters.

FIGS. 29A-29B are graphs showing the differences in (FIG. 29A) NETest score and (FIG. 29B) circulating CgA levels, each in in stable disease (SD) conditions and somatostatin analog (SSA) treatment failure (equivalent of PD conditions).

FIGS. 30A-30D are graphs showing the differences in gene-derived algorithms, (FIG. 30A) PDA, (FIG. 30B) NDA, (FIG. 30C) TDA, and (FIG. 30D) IDA, in stably treated patients (SD) and treatment failure (PD).

FIGS. 31A-31I are graphs showing the differences in gene-derived clusters, specifically (FIG. 31A) SSTrome, (FIG. 31B) Proliferome, (FIG. 31C) Signalome, (FIG. 31D) Metabolome, (FIG. 31E) Secretome, (FIG. 31F) Secretome, (FIG. 31G) Plurome, (FIG. 31H) EpiGenome, and (FIG. 31I) ApopTome, in stably treated patients (SD) and SSA treatment failure (equivalent of PD conditions).

FIGS. 32A-32B are graphs showing a ROC analysis according to (FIG. 32A) gene-derived cluster algorithms and (FIG. 32B) gene clusters for differentiating treatment failure (equivalent of PD conditions) from controls.

FIGS. 33A-33B are graphs showing the percentage of correct calls for each of (FIG. 33A) the gene-derived cluster algorithms and clusters for defining treatment failure in patients categorized as SD and (FIG. 33B) a Best-of-3 outperformed by a combination of SSTRome and Proliferome.

FIG. 34 is a nomogram for somatostatin analog treated patients including the mathematically-derived score as well as the SSTRome, Proliferome, and their combination.

FIGS. 35A-35B are graphs that demonstrate therapeutic efficacy of SSAs and the proportion of patients with low/high NETest scores that developed disease recurrence.

FIGS. 36A-36B are graphs that demonstrate the time point when either the NETest was elevated (>80%) or CgA was abnormal prior to the development of image positive disease recurrence as well as the times that these events occurred prior to image-positive disease recurrence.

FIGS. 37A-37D are graphs that demonstrate the NETest scores prior to long-term follow up (FIG. 37A), and the times to relapse in patients with elevated scores (FIGS. 37B, 37D) or disease-free time (FIG. 37C).

FIGS. 38A-38F are graphs including predicted Ki67 index versus Ki67 index in (FIGS. 38A-38B) SSTRome, (FIGS. 38C-38E) All genes, and (FIGS. 38D-38F) high relevant genes (KRAS, SSTR4, and VPS13C).

FIGS. 39A-39F are graphs showing the correlations (linear regression) between gene clusters or algorithms, (FIG. 39A) SSTRome and Ki67, (FIG. 39B) TDA and Ki67, (FIG. 39C) Proliferome and Ki67, (FIG. 39D) PDA and Ki67, (FIG. 39E) IDA and Ki67, and (FIG. 39F) PDA and Ki67, each versus the Ki-67 index.

FIGS. 40A-40D are graphs modeling predicted SUVmax (tumor uptake—a measure of receptor density/target availability) for SSTRome (Group I: (FIG. 40A) and (FIG. 40C)) and all genes (Group II: (FIG. 40B) and (FIG. 40D)).

FIGS. 41A-41F are graphs modeling MTV (molecular tumor volume—a measure of the tumor burden) in individual genes (FIGS. 41A-41B), SSTRome (FIGS. 41C-41E), and all genes (FIGS. 41D-41F).

FIGS. 42A-42C are graphs showing ZFHX3 expression in patients identified with (FIG. 42A) new lesions by imaging, with (FIG. 42B) progressive disease by RECIST, and (FIG. 42C) following surgery.

FIGS. 43A-43B are graphs showing ZFHX3 expression in (FIG. 43A) patients who remain in a stable disease state of GEP-NEN versus (FIG. 43B) those who develop a progressive disease state of GEP-NEN.

FIGS. 44A-44C are graphs representing (FIG. 44A) the effectiveness of peptide receptor radionucleotide therapy (PRRT), (FIG. 44B) changes in NETest Score versus clinical status at 6M Follow-up (FuP) in responders (R) and non-responders (NR); and (FIG. 44C) change in CgA level versus clinical status at 6M FuP.

FIG. 45 are graphs showing concordance between the NETest in responders and non-responders prior to and after therapy and in addition the comparison to CgA.

FIGS. 46A-46B shows the accuracy of the NETest Score versus CgA for treatment responses.

FIG. 47A-47D shows expression of two subsets of NETest genes, the signalome and metabolome in blood samples prior to therapy and the differences between responders (R) and non-responders (NR), the predictive utility of each as well as when combined into a biological index (FIG. 47B), the utility for predicting treatment response alone (Quotient) or as a combination with grade (Combination) (FIG. 47C) and the metrics of the combination for predicting the outcome of PRRT therapy (FIG. 47D).

### DETAILED DESCRIPTION OF THE INVENTION

Three-quarters of all human genes undergo alternative splicing. Identifying and defining cancer-specific splice variants is therefore advantageous for the development of biomarker assays. The described embodiments derive from the surprising discovery that particular cancer-specific splice variants of NET marker genes can be used to maximize the difference between neoplasia and normal samples in biomarker diagnostic methods.

The present invention provides a method for detecting a gastroenteropancreatic neuroendocrine neoplasm (GEP-NEN) in a subject in need thereof, including determining the expression level of at least 22 biomarkers from a test sample from the subject by contacting the test sample with a plurality of agents specific to detect the expression of the at least 22 biomarkers, wherein the 22 biomarkers are selected from the group consisting of APLP2, ARAF, ATP6V1H, BNIP3L, BRAF, CD59, COMMD9, CTGF, FZD7, GLT8D1, KRAS, MKI67/KI67, MORF4L2, NAPILI, NOL3, OAZ2, PANK2, PHF21A, PLD3, PNMA2, PQBP1, RAF1, RNF41, RSF1, SLC18A1/VMAT1, SLC18A2/VMAT2, SMARCD3, SPATA7, SSTR1, SSTR3, SSTR4, SSTR5, TECPR2, TPH1, TRMT112, WDFY3, ZFHX3 and ZZZ3; determining the expression level of the at least 22 biomarkers from a reference sample by contacting the reference sample with a plurality of agents specific to detect the expression of the at least 22 biomarkers; normalizing the expression level of the at least 22 biomarkers in the test sample to the expression level of the at least 22 biomarkers in the reference sample; comparing the normalized expression level of the at least 22 biomarkers in the test sample with a predetermined cutoff value; determining the presence of a GEP-NEN in the subject when the normalized expression level is equal to or greater than the predetermined cutoff value or determining the absence of a GEP-NEN in the subject when the normalized expression level is below the predetermined cutoff value, wherein the predetermined cutoff value is 2 on a MAARC-NET scoring system scale of 0-8, or 0% on a scale of 0-100%.

The score is based on a “majority vote” strategy and was developed from a binary classification system whereby a sample will be called “normal” and given a score of 0 or “tumor” and will be scored “1”. The score can range from 0 (four calls all “normal”) to 4 (four calls all “tumor”). Each “call” is the binary result (either “0” for normal or “1” for tumor) of one of four different learning algorithms: Support Vector Machine (SVM), Linear Discrimination Analysis (LDA), K-Nearest Neighbor (KNN), and Naïve Bayes (Bayes). Each of these four learning algorithms were trained on an internal training set including 67 controls and 63 GEP-NEN. In this training set, differentially expressed genes (control versus GEP-NEN) were identified as significant using a t-test. Based upon the training set, each of the learning algorithms were trained to differentiate between normal and tumor gene expression to within a level of significance of at least p<0.05. According to the majority voting strategy, those samples with less than 2 “normal” calls are classified as GEP-NEN.

The at least 22 biomarkers can include APLP2, ARAF, CD59, CTGF, FZD7, KRAS, MKI67/KI67, MORF4L2, NAPILI, NOL3, PNMA2, RAF1, RSF1, SLC18A2/VMAT2, SMARCD3, SPATA7, SSTR1, SSTR3, SSTR4, SSTR5, TPH1, TRMT112, and ZFHX3.

The methods can further include determining the presence of a progressive GEP-NEN in the subject when the normalized expression level is equal to or higher than the predetermined cutoff value, wherein the predetermined cutoff value is 5 on a scale of 0-8, or less than 55% on a scale of 0-100%.

The methods can further include identifying a level of risk for the subject to develop a progressive GEP-NEN the method further including identifying a low level of risk for developing a progressive GEP-NEN when the normalized expression level is less than a predetermined cutoff value of 5 on a scale of 0-8, or less than 55% on a scale of 0-100%; identifying an intermediate level of risk for developing a progressive GEP-NEN when the normalized expression level is equal to or greater than a predetermined cutoff value of 5 and less than a predetermined cutoff value of 7 on a scale of 0-8, or equal to or greater than 55% and less than 75% on a scale of 0-100%; or identifying a high level of risk for developing a progressive GEP-NEN when the normalized expression level is equal to or greater than a predetermined cutoff value of 7 on a scale of 0-8, or equal to or greater than 75% on a scale of 0-100%.

The biomarker can be RNA, cDNA, or protein. When the biomarker is RNA, the RNA can be reverse transcribed to produce cDNA (such as by RT-PCR, and the produced cDNA expression level is detected. The expression level of the biomarker can be detected by forming a complex between the biomarker and a labeled probe or primer. When the biomarker is RNA or cDNA, the RNA or cDNA detected by forming a complex between the RNA or cDNA and a labeled nucleic acid probe or primer. The complex between the RNA or cDNA and the labeled nucleic acid probe or primer can be a hybridization complex. When the biomarker is protein, the protein can be detected by forming a complex between the protein and a labeled antibody. The label can be any label for example a fluorescent label, chemiluminescence label, radioactive label, etc.

The test sample can be any biological fluid obtained from the subject. Preferably, the test sample is blood, serum, plasma or neoplastic tissue. The reference sample can be any biological fluid obtained from a subject not having, showing symptoms of or diagnosed with a neoplastic disease. Preferably, the reference sample is blood, serum, plasma or non-neoplastic tissue.

The subject in need thereof can be a subject diagnosed with a GEP-NEN, a subject having at least one GEP-NEN symptom or a subject having a predisposition or familial history for developing a GEP-NEN. The subject can be any mammal. Preferably, the subject is human. The terms subject and patient are used interchangeably herein.

The methods can further include treating a subject identified as having an intermediate level or high level of risk for developing a progressive GEP-NEN with surgery or drug therapy. The drug therapy can be somatostatin analog treatment or peptide receptor radiotherapy therapy (PRRT). The methods can further include treating a subject identified as having a low level of risk for developing a progressive GEP-NEN with regular or periodic monitoring over at least a six month period, a twelve month period, an eighteen month period or twenty four month period.

The present invention also provides a method for differentiating stable and progressive GEP-NEN in a subject comprising determining that the normalized expression level of the at least 22 biomarkers from the test sample from the subject is equal to or greater than a predetermined cutoff value of 5 and less than a predetermined cutoff value of 6, according to the methods of the present invention; detecting an expression level of SMARCD3 and TPH1 from the test sample and from a reference sample by contacting the test sample and the reference sample with a plurality of agents specific to detect the expression of SMARCD3 and the expression of TPH1; normalizing the expression level of SMARCD3 and TPH1 in the test sample to the expression level of SMARCD3 and TPH1 in the reference sample; comparing the normalized expression level of SMARCD3 and TPH1 in the test sample with a first and a second predetermined cutoff value, respectively; and determining the presence of stable GEP-NEN in the subject when the normalized expression level of SMARCD3 is greater than the first predetermined cutoff value and the expression level of TPH1 is equal to or greater than the second predetermined cutoff value, or determining the presence of progressive GEP-NEN in the subject when the normalized expression level of SMARCD3 is equal to or less than the first predetermined cutoff value and the expression level of TPH1 is less than the second predetermined cutoff value wherein the first predetermined cutoff value is 1.3 on a scale of 0-8 and wherein the second predetermined cutoff value is 4 on a scale of 0-8.

The first predetermined cutoff value of 1.3 corresponds to 12% on a scale of 0-100% and wherein the second predetermined cutoff value of 4 corresponds to 41% on a scale of 0-100%.

The present invention also provides a method for differentiating stable and progressive GEP-NEN in a subject comprising determining that the normalized expression level of the at least 22 biomarkers from the test sample from the subject is equal to or greater than a predetermined cutoff value of 6 and less than a predetermined cutoff value of 7, according to the methods of the present invention; detecting an expression level of VMAT1 and PHF21A from the test sample and from a reference sample by contacting the test sample and reference sample with a plurality of agents specific to detect the expression of VMAT1 and the expression of PHF21A, normalizing the expression level of VMAT1 and PHF21A in the test sample to the expression level of VMAT1 and PHF21A in the reference sample; comparing the normalized expression level of VMAT1 and PHF21A in the test sample with a first and a second predetermined cutoff value, respectively; and determining the presence of stable GEP-NEN in the subject when the normalized expression level of VMAT1 is equal to or greater than the first predetermined cutoff value and the expression level of PHF21A is less than the second predetermined cutoff value, or determining the presence of progressive GEP-NEN in the subject when the normalized expression level of VMAT1 is equal to or greater than the first predetermined cutoff value and the expression level of PHF21A is equal to or greater than the second predetermined cutoff value wherein the first predetermined cutoff value is 0 on a scale of 0-8 and wherein the second predetermined cutoff value is 1.2 on a scale of 0-8.

The first predetermined cutoff value of 0 corresponds to 0% on a scale of 0-100% and wherein the second predetermined cutoff value of 1.2 corresponds to 8% on a scale of 0-100%.

The present invention also provides a method for differentiating stable and progressive GEP-NEN in a subject comprising determining that the normalized expression level of the at least 22 biomarkers from the test sample from the subject is equal to or greater than a predetermined cutoff value of 7 and less than a predetermined cutoff value of 8, according to the methods of the present invention; detecting an expression level of VMAT1 and PHF21A from the test sample and a reference sample by contacting the test sample and the reference sample with a plurality of agents specific to detect the expression of VMAT1 and the expression of PHF21A; normalizing the expression level of VMAT1 and PHF21A in the test sample to the expression level of VMAT1 and PHF21A in the reference sample; comparing the normalized expression level of VMAT1 and PHF21A in the test sample with a first and a second predetermined cutoff value, respectively; and determining the presence of stable GEP-NEN in the subject when the normalized expression level of VMAT1 is equal to or greater than the first predetermined cutoff value and the expression level of PHF21A is greater than the second predetermined cutoff value, or determining the presence of progressive GEP-NEN in the subject when the normalized expression level of VMAT1 is equal to or greater than the first predetermined cutoff value and the expression level of PHF21A is equal to or less than the second predetermined cutoff value wherein the first predetermined cutoff value is 0 on a scale of 0-8 and wherein the second predetermined cutoff value is 1 on a scale of 0-8.

The first predetermined cutoff value of 0 corresponds to 0% on a scale of 0-100% and wherein the second predetermined cutoff value of 1 corresponds to 7% on a scale of 0-100%.

The present invention also provides a method for differentiating stable and progressive GEP-NEN in a subject comprising determining that the normalized expression level of the at least 22 biomarkers from the test sample from the subject is equal to a predetermined cutoff value of 8, according to the methods of the present invention; detecting an expression level of ZZZ3 from the test sample and a reference sample by contacting the test sample and the reference sample with at least one agent specific to detect the expression of ZZZ3; normalizing the expression level of ZZZ3 in the test sample to the expression level of ZZZ3 in the reference sample; comparing the normalized expression level of ZZZ3 in the test sample with a predetermined cutoff value; and determining the presence of progressive GEP-NEN in the subject when the normalized expression level of ZZZ3 is equal to or less than the predetermined cutoff value, wherein the predetermined cutoff value is 1 on a scale of 0-8.

The predetermined cutoff value of 1 corresponds to 18% on a scale of 0-100%.

The methods of the present invention further include determining the expression level of each of 16 biomarkers from a test sample from the subject and a reference sample by contacting the test sample and the reference sample with a plurality of agents specific to detect the expression of each of the 16 biomarkers, wherein the 16 biomarkers comprise Ki67, NAPILI, NOL3, TECPR2, ARAF1, BRAF, KRAS, RAF1, PQBP1, TPH1, COMMD9, MORF4L2, RNF41, RSF1, SMARCD3, and ZFHX3; summing the expression level of each of the 16 biomarkers of the test sample to generate a progressive diagnostic I total test value and summing the expression level of each of the 16 biomarkers of the reference sample to generate a progressive diagnostic I total reference value, wherein an increased value of the progressive diagnostic I total test value compared to the progressive diagnostic I total reference value indicates the presence of progressive GEP-NEN in the subject.

The methods of the present invention further include determining the expression level of each of 15 biomarkers from a test sample from the subject and a reference sample by contacting the test sample and the reference sample with a plurality of agents specific to detect the amount of each of the 15 biomarkers, wherein the 15 biomarkers comprise ARAF1, BRAF, KRAS, RAF1, Ki67, NAPILI, NOL3, GLT8D1, PLD3, PNMA2, VMAT2, TPH1, FZD7, MORF4L2 and ZFHX3; averaging the expression level of each of the 15 biomarkers of the test sample to generate a progressive diagnostic II test value and averaging the expression level of each of the 15 biomarkers of the reference sample to generate a progressive diagnostic II reference value, wherein an increased value of the progressive diagnostic II test value compared to the progressive diagnostic II reference value indicates the presence of progressive GEP-NEN in the subject.

The methods of the present invention further include determining the expression level of each of 7 biomarkers from a test sample from the subject and a reference sample by contacting the test sample and the reference sample with a plurality of agents specific to detect the amount of each of the 7 biomarkers, wherein the 7 biomarkers comprise PNMA2, VMAT2, COMMD9, SSTR1, SSTR3, SSTR4, and SSTR5; summing the expression level of each of the 7 biomarkers of the test sample to generate a progressive diagnostic III total test value and summing the expression level of each of the 7 biomarkers of the reference sample to generate a progressive diagnostic III total reference value, wherein an increased value of the progressive diagnostic III total test value compared to the progressive diagnostic III total reference value indicates the presence of progressive GEP-NEN in the subject.

The methods of the present invention further include determining the expression level of each of 11 biomarkers from a test sample from the subject and a reference sample by contacting the test sample and the reference sample with a plurality of agents specific to detect the amount of each of the 11 biomarkers, wherein the 11 biomarkers comprise Ki67, NAPILI, NOL3, TECPR2, PQBP1, TPH1, MORF4L2, RNF41, RSF1, SMARCD3, and ZFHX3; summing the expression level of each of the 11 biomarkers of the test sample to generate a progressive diagnostic IV total test value and summing the expression level of each of the 11 biomarkers of the reference sample to generate a progressive diagnostic IV total reference value, wherein an increased value of the progressive diagnostic IV total test value compared to the progressive diagnostic IV total reference value indicates the presence of progressive GEP-NEN in the subject.

The present invention also provides a method for determining the risk of relapsing or reoccurring progressive GEP-NEN in a post-surgery subject, including determining the expression level of at least 22 biomarkers from a test sample from the subject by contacting the test sample with a plurality of agents specific to detect the expression of the at least 22 biomarkers, wherein the 22 biomarkers are selected from the group consisting of APLP2, ARAF, ATP6V1H, BNIP3L, BRAF, CD59, COMMD9, CTGF, FZD7, GLT8D1, KRAS, MKI67/KI67, MORF4L2, NAPILI, NOL3, OAZ2, PANK2, PHF21A, PLD3, PNMA2, PQBP1, RAF1, RNF41, RSF1, SLC18A1/VMAT1, SLC18A2/VMAT2, SMARCD3, SPATA7, SSTR1, SSTR3, SSTR4, SSTR5, TECPR2, TPH1, TRMT112, WDFY3, ZFHX3 and ZZZ3; determining the expression level of the at least 22 biomarkers from a reference sample by contacting the reference sample with a plurality of agents specific to detect the expression of the at least 22 biomarkers; normalizing the expression level of the at least 22 biomarkers in the test sample to the expression level of the at least 22 biomarkers in the reference sample; comparing the normalized expression level of the at least 22 biomarkers in the test sample with a predetermined cutoff value; identifying an absence of risk of relapsing or reoccurring progressive GEP-NEN post-surgery when the normalized expression level is less than a predetermined cutoff value of 2 on a scale of 0-8, or less than 0% on a scale of 0-100%; identifying a low level of risk of relapsing or reoccurring progressive GEP-NEN post-surgery when the normalized expression level is less than a predetermined cutoff value of 5 on a scale of 0-8, or less than 55% on a scale of 0-100%; identifying an intermediate level of risk of relapsing or reoccurring progressive GEP-NEN post-surgery when the normalized expression level is equal to or greater than a predetermined cutoff value of 5 and less than a predetermined cutoff value of 7 on a scale of 0-8, or equal to or greater than 55% and less than 75% on a scale of 0-100%; or identifying a high level of risk of relapsing or reoccurring progressive GEP-NEN post-surgery when the normalized expression level is equal to or greater than a predetermined cutoff value of 7 on a scale of 0-8, or equal to or greater than 75% on a scale of 0-100%.

The present invention also provides a method for determining the risk of relapsing or reoccurring progressive GEP-NEN in a subject treated with somatostatin, including determining the expression level of at least 22 biomarkers from a test sample from the subject by contacting the test sample with a plurality of agents specific to detect the expression of the at least 22 biomarkers, wherein the 22 biomarkers are selected from the group consisting of APLP2, ARAF, ATP6V1H, BNIP3L, BRAF, CD59, COMMD9, CTGF, FZD7, GLT8D1, KRAS, MKI67/KI67, MORF4L2, NAPILI, NOL3, OAZ2, PANK2, PHF21A, PLD3, PNMA2, PQBP1, RAF1, RNF41, RSF1, SLC18A1/VMAT1, SLC18A2/VMAT2, SMARCD3, SPATA7, SSTR1, SSTR3, SSTR4, SSTR5, TECPR2, TPH1, TRMT112, WDFY3, ZFHX3 and ZZZ3; determining the expression level of the at least 22 biomarkers from a reference sample by contacting the reference sample with a plurality of agents specific to detect the expression of the at least 22 biomarkers; normalizing the expression level of the at least 22 biomarkers in the test sample to the expression level of the at least 22 biomarkers in the reference sample; comparing the normalized expression level of the at least 22 biomarkers in the test sample with a predetermined cutoff value; determining the presence of a GEP-NEN in the subject when the normalized expression level is equal to or greater than the predetermined cutoff value or determining the absence of a GEP-NEN in the subject when the normalized expression level is below the predetermined cutoff value, wherein the predetermined cutoff value is 2 on a MAARC-NET scoring system scale of 0-8, or 0% on a scale of 0-100%; when a GEP-NEN is present, determining the expression level of each of 8 biomarkers from a test sample from the subject and a reference sample by contacting the test sample and the reference sample with a plurality of agents specific to detect the expression of each of the 8 biomarkers, wherein the 8 biomarkers comprise Ki67, NAPILI, NOL3, TECPR2, SSTR1, SSTR2, SSTR4, and SSTR5; summing the expression level of each of the 8 biomarkers of the test sample to generate a progressive diagnostic V total test value and summing the expression level of each of the 8 biomarkers of the reference sample to generate a progressive diagnostic V total reference value, wherein an increased value of the progressive diagnostic V total test value compared to the progressive diagnostic V total reference value indicates the presence of relapsing or reoccurring progressive GEP-NEN in the subject.

The present invention also provides a method for determining a response of a peptide receptor radionucleotide therapy (PRRT) of a GEP-NEN in a subject in need thereof, including determining the expression level of each of 8 biomarkers from a test sample from the subject and a reference sample by contacting the test sample and the reference sample with a plurality of agents specific to detect the expression of each of the 8 biomarkers, wherein the 8 biomarkers comprise ARAF1, BRAF, KRAS, RAF1, ATP6V1H, OAZ2, PANK2, PLD3; normalizing the expression level of the 8 biomarkers in the test sample to the expression level of the 8 biomarkers in the reference sample; comparing the normalized expression level of the 8 biomarkers in the test sample with a predetermined cutoff value; determining the presence of a PRRT-responsive GEP-NEN in the subject when the normalized expression level of the 8 biomarkers is greater than a predetermined cutoff value, wherein the predetermined cutoff value is 5.9 on a scale of 0-8.

The present invention also provides a method for determining a response of a peptide receptor radionucleotide therapy (PRRT) of a GEP-NEN in a subject in need thereof, including (a) following a first cycle of PRRT therapy: determining the expression level of at least 22 biomarkers from a first cycle test sample from the subject by contacting the first cycle test sample with a plurality of agents specific to detect the expression of the at least 22 biomarkers, wherein the 22 biomarkers are selected from the group consisting of APLP2, ARAF, ATP6V1H, BNIP3L, BRAF, CD59, COMMD9, CTGF, FZD7, GLT8D1, KRAS, MKI67/KI67, MORF4L2, NAPILI, NOL3, OAZ2, PANK2, PHF21A, PLD3, PNMA2, PQBP1, RAF1, RNF41, RSF1, SLC18A1/VMAT1, SLC18A2/VMAT2, SMARCD3, SPATA7, SSTR1, SSTR3, SSTR4, SSTR5, TECPR2, TPH1, TRMT112, WDFY3, ZFHX3 and ZZZ3; determining the expression level of the at least 22 biomarkers from a reference sample by contacting the reference sample with a plurality of agents specific to detect the expression of the at least 22 biomarkers; normalizing the expression level of the at least 22 biomarkers in the first cycle test sample to the expression level of the at least 22 biomarkers in the reference sample; (b) following a second cycle of PRRT therapy, determining the expression level of at least 22 biomarkers from a second cycle test sample from the subject by contacting the test sample with a plurality of agents specific to detect the expression of the at least 22 biomarkers, wherein the 22 biomarkers are selected from the group consisting of APLP2, ARAF, ATP6V1H, BNIP3L, BRAF, CD59, COMMD9, CTGF, FZD7, GLT8D1, KRAS, MKI67/KI67, MORF4L2, NAPILI, NOL3, OAZ2, PANK2, PHF21A, PLD3, PNMA2, PQBP1, RAF1, RNF41, RSF1, SLC18A1/VMAT1, SLC18A2/VMAT2, SMARCD3, SPATA7, SSTR1, SSTR3, SSTR4, SSTR5, TECPR2, TPH1, TRMT112, WDFY3, ZFHX3 and ZZZ3; determining the expression level of the at least 22 biomarkers from a reference sample by contacting the reference sample with a plurality of agents specific to detect the expression of the at least 22 biomarkers; normalizing the expression level of the at least 22 biomarkers in the second cycle test sample to the expression level of the at least 22 biomarkers in the reference sample; (c) determining a ratio of change of the normalized expression levels from (a) to the normalized expression levels from (b); (d) determining the presence of a PRRT-responsive GEP-NEN when the ratio of change is greater than a pre-PRRT therapy cutoff value, wherein the pre-PRRT therapy cutoff value is 1 on a scale of 0-8.

The present invention also provides a method for determining a progression of a GEP-NEN in a subject in need thereof, including determining the expression level of ZFHX3 from a test sample from the subject by contacting the test sample with an agent specific to detect the expression of ZFHX3; determining the expression level of ZFHX3 from a reference sample by contacting the reference sample with an agent specific to detect the expression of ZFHX3; normalizing the expression level of ZFHX3 in the test sample to the expression level of ZFHX3 in the reference sample; comparing the normalized expression level of ZFHX3 in the test sample with a predetermined cutoff value; determining the progression of a GEP-NEN in the subject when the normalized expression level is equal to or greater than the predetermined cutoff value, wherein the predetermined cutoff value is 0.5 on a scale of 0-8.

The present invention also provides a method for predicting tumor proliferation of a GEP-NEN in a subject in need thereof, including (a) determining the expression level of at least 22 biomarkers from a test sample from the subject by contacting the test sample with a plurality of agents specific to detect the expression of the at least 22 biomarkers, wherein the 22 biomarkers are selected from the group consisting of APLP2, ARAF, ATP6V1H, BNIP3L, BRAF, CD59, COMMD9, CTGF, FZD7, GLT8D1, KRAS, MKI67/KI67, MORF4L2, NAPILI, NOL3, OAZ2, PANK2, PHF21A, PLD3, PNMA2, PQBP1, RAF1, RNF41, RSF1, SLC18A1/VMAT1, SLC18A2/VMAT2, SMARCD3, SPATA7, SSTR1, SSTR3, SSTR4, SSTR5, TECPR2, TPH1, TRMT112, WDFY3, ZFHX3 and ZZZ3; determining the expression level of the at least 22 biomarkers from a reference sample by contacting the reference sample with a plurality of agents specific to detect the expression of the at least 22 biomarkers; normalizing the expression level of the at least 22 biomarkers in the test sample to the expression level of the at least 22 biomarkers in the reference sample; comparing the normalized expression level of the at least 22 biomarkers in the test sample with a predetermined cutoff value; determining the presence of a GEP-NEN in the subject when the normalized expression level is equal to or greater than the predetermined cutoff value or determining the absence of a GEP-NEN in the subject when the normalized expression level is below the predetermined cutoff value, wherein the predetermined cutoff value is 2 on a MAARC-NET scoring system scale of 0-8, or 0% on a scale of 0-100%; (b) when a GEP-NEN is present, determining the expression level of each of 3 biomarkers from a test sample from the subject and a reference sample by contacting the test sample and the reference sample with a plurality of agents specific to detect the expression of each of the 3 biomarkers, wherein the 3 biomarkers comprise KRAS, SSTR4 and VPS13C; summing the expression level of each of the 3 biomarkers of the test sample to generate a progressive diagnostic VI total test value and summing the expression level of each of the 3 biomarkers of the reference sample to generate a progressive diagnostic VI total reference value, wherein an increased value of the progressive diagnostic VI total test value compared to the progressive diagnostic VI total reference value indicates the presence of tumor proliferation of a GEP-NEN in the subject.

The method wherein (b) further includes determining the expression level of each of 3 biomarkers from a test sample from the subject and a reference sample by contacting the test sample and the reference sample with a plurality of agents specific to detect the expression of each of the 3 biomarkers, wherein the 3 biomarkers comprise SSTR1, SSTR2 and SSTR5; summing the expression level of each of the 3 biomarkers of the test sample to generate a progressive diagnostic VII total test value and summing the expression level of each of the 3 biomarkers of the reference sample to generate a progressive diagnostic VII total reference value, wherein an increased value of the progressive diagnostic VII total test value compared to the progressive diagnostic VII total reference value indicates the presence of tumor proliferation of a GEP-NEN in the subject.

As used herein, the term “GEP-NEN biomarker” and “NET biomarker” refer synonymously to a biological molecule, such as a gene product, the expression or presence of which (e.g., the expression level or expression profile) on its own or as compared to one or more other biomarkers (e.g., relative expression) differs (i.e., is increased or decreased) depending on the presence, absence, type, class, severity, metastasis, location, stage, prognosis, associated symptom, outcome, risk, likelihood or treatment responsiveness, or prognosis of GEP-NEN disease, or is associated positively or negatively with such factors of the prediction thereof.

As used herein, the term “polynucleotide” or nucleic acid molecule means a polymeric form of nucleotides of at least 10 bases or base pairs in length, either ribonucleotides or deoxynucleotides or a modified form of either type of nucleotide, and is meant to include single and double stranded forms of DNA. As used herein, a nucleic acid molecule or nucleic acid sequence that serves as a probe in a microarray analysis preferably comprises a chain of nucleotides, more preferably DNA and/or RNA. In other embodiments, a nucleic acid molecule or nucleic acid sequence comprises other kinds of nucleic acid structures such a for instance a DNA/RNA helix, peptide nucleic acid (PNA), locked nucleic acid (LNA) and/or a ribozyme. Hence, as used herein the term “nucleic acid molecule” also encompasses a chain comprising non-natural nucleotides, modified nucleotides and/or non-nucleotide building blocks which exhibit the same function as natural nucleotides.

As used herein, the terms “hybridize,” “hybridizing”, “hybridizes,” and the like, used in the context of polynucleotides, are meant to refer to conventional hybridization conditions, preferably such as hybridization in 50% formamide/6×SSC/0.1% SDS/100 μg/ml ssDNA, in which temperatures for hybridization are above 37 degrees and temperatures for washing in 0.1×SSC/0.1% SDS are above 55 degrees C., and most preferably to stringent hybridization conditions.

The term “blood biopsy” refers to a diagnostic study of the blood to determine whether a patient presenting with symptoms has a condition that may be classified as either benign (low activity) or malignant (high activity/metastatic).

The term “classifying” as used herein with regard to different types or stages of GEP-NEN refers to the act of compiling and analyzing expression data for using statistical techniques to provide a classification to aid in diagnosis of a stage or type of GEP-NEN.

The term “classifier” as used herein refers to an algorithm that discriminates between disease states with a predetermined level of statistical significance. A two-class classifier is an algorithm that uses data points from measurements from a sample and classifies the data into one of two groups. A multi-class classifier is an algorithm that uses data points from measurements from a sample and classifies the data into one of multiple groups. The “classifier” maximizes the probability of distinguishing a randomly selected cancer sample from a randomly selected benign sample, i.e., the area under a curve (AUC) of receiver operating characteristic (ROC) curve.

The term “normalization” or “normalizer” as used herein refers to the expression of a differential value in terms of a standard value to adjust for effects which arise from technical variation due to sample handling, sample preparation and mass spectrometry measurement rather than biological variation of protein concentration in a sample. For example, when measuring the expression of a differentially expressed protein, the absolute value for the expression of the protein can be expressed in terms of an absolute value for the expression of a standard protein that is substantially constant in expression.

The term “condition” as used herein refers generally to a disease, event, or change in health status.

The terms “diagnosis” and “diagnostics” also encompass the terms “prognosis” and “prognostics”, respectively, as well as the applications of such procedures over two or more time points to monitor the diagnosis and/or prognosis over time, and statistical modeling based thereupon. Furthermore the term diagnosis includes: a. prediction (determining if a patient will likely develop aggressive disease (hyperproliferative/invasive)), b. prognosis (predicting whether a patient will likely have a better or worse outcome at a pre-selected time in the future), c. therapy selection, d. therapeutic drug monitoring, and e. relapse monitoring.

The term “providing” as used herein with regard to a biological sample refers to directly or indirectly obtaining the biological sample from a subject. For example, “providing” may refer to the act of directly obtaining the biological sample from a subject (e.g., by a blood draw, tissue biopsy, lavage and the like). Likewise, “providing” may refer to the act of indirectly obtaining the biological sample. For example, providing may refer to the act of a laboratory receiving the sample from the party that directly obtained the sample, or to the act of obtaining the sample from an archive.

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures.

The term “biological sample” as used herein refers to any sample of biological origin potentially containing one or more biomarker proteins. Examples of biological samples include tissue, organs, or bodily fluids such as whole blood, plasma, serum, tissue, lavage or any other specimen used for detection of disease.

The term “subject” as used herein refers to a mammal, preferably a human.

“Treating” or “treatment” as used herein with regard to a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

Biomarker levels may change due to treatment of the disease. The changes in biomarker levels may be measured by the present invention. Changes in biomarker levels may be used to monitor the progression of disease or therapy.

“Altered”, “changed” or “significantly different” refer to a detectable change or difference from a reasonably comparable state, profile, measurement, or the like. Such changes may be all or none. They may be incremental and need not be linear. They may be by orders of magnitude. A change may be an increase or decrease by 1%, 5%, 10%, 20%, 30%, 40%, 50%, 60%, 70%, 80%, 90%, 95%, 99%, 100%, or more, or any value in between 0% and 100%. Alternatively the change may be 1-fold, 1.5-fold 2-fold, 3-fold, 4-fold, 5-fold or more, or any values in between 1-fold and five-fold. The change may be statistically significant with a p value of 0.1, 0.05, 0.001, or 0.0001.

The term “disease prevalence” refers to the number of all new and old cases of a disease or occurrences of an event during a particular period. Prevalence is expressed as a ratio in which the number of events is the numerator and the population at risk is the denominator.

The term “disease incidence” refers to a measure of the risk of developing some new condition within a specified period of time; the number of new cases during some time period, it is better expressed as a proportion or a rate with a denominator.

The term “stable disease” refers to a diagnosis for the presence of GEP-NEN, however GEP-NEN has been treated and remains in a stable condition, i.e. one that that is not progressive, as determined by imaging data and/or best clinical judgment.

The term “progressive disease” refers to a diagnosis for the presence of a highly active state of GEP-NEN, i.e. one has not been treated and is not stable or has been treated and has not responded to therapy, or has been treated and active disease remains, as determined by imaging data and/or best clinical judgment.

The term “expression level score” or “NETest score” refers to the output of a mathematically-derived classifier algorithm generated from the combination of classification algorithms, i.e. SVM, LDA, KNN, and Bayes. This score ranges between 0 and 100%. The expression level score from a test sample, once compared to the expression level score for a reference or control sample, may be used to diagnose the presence of GEP-NEN, the different stages of GEP-NEN, predict the risk of contracting a stage of GEP-NEN, or determines the risk of recurrence of GEP-NEN in post-therapy human patients. Distinctions between GEP-NEN disease states are based on pre-determined expression level score thresholds and/or ranges as further defined in the present application.

Diagnosis and prognosis of GEP-NEN has been difficult, in part due to the prosaic symptoms and syndromes of the disease, such as carcinoid syndrome, diarrhea, flushing, sweating, bronchoconstriction, gastrointestinal bleeding, cardiac disease, intermittent abdominal pain, which often remain silent for years. Available diagnostic methods include anatomical localization, such as by imaging, e.g., X-ray, gastrointestinal endoscopy, abdominal computed tomography (CT), combined stereotactic radiosurgery (SRS)/CT, and MRI, and detection of some gene products e.g., chromogranin A. Known methods are limited, for example by low specificity and/or sensitivity and/or in the ability to detect early-stage disease.

Detection of single biomarkers has not been entirely satisfactory, for example, to identify malignancy in human blood samples and to predict complex outcomes like fibrosis and metastasis. See Michiels S, Koscielny S, Hill C, “Interpretation of microarray data in cancer,” Br J Cancer 2007; 96 (8): 1155-8. Limitations in available methods have contributed to difficulties in pathological classification, staging, and prediction, treatment developing and monitoring therapeutic effects. Among the embodiments provided herein are methods and compositions that address these limitations.

In one aspect, the present application relates to the detection and identification of GEP-NEN biomarkers and panels of such biomarkers, for example, in biological samples. Provided are methods and compositions (e.g., agents, such as polynucleotides), for detecting, determining expression levels of, and recognizing or binding to the biomarkers, in biological samples, typically blood samples.

Also provided are models and biomathematical algorithms, e.g., supervised learning algorithms, and methods using the same, for prediction, classification, and evaluation of GEP-NEN and associated outcomes, for example, predicting degree of risk, responsiveness to treatment, metastasis or aggressiveness, and for determining GEP-NEN subtype.

Detection of the biomarkers using the provided embodiments is useful for improving GEP-NEN diagnostics and prognostics, and to inform treatment protocols. In some aspects, detection of the biomarkers and/or expression levels by the provided embodiments confirms or indicates the presence, absence, stage, class, location, sub-type, aggressiveness, malignancy, metastasis, prognosis, or other outcome of GEP-NEN, or a GEP-NEN cell, such as a circulating GEP-NEN cell (CNC). The provided methods and compositions may be used for tumor localization, and for predicting or detecting metastases, micrometastases, and small lesions, and/or for determining degree of risk, likelihood of recurrence, treatment responsiveness or remission, and informing appropriate courses of treatment. For example, detecting the biomarkers, e.g., in circulation may be used to detect early-stage and primary GEP-NENs (e.g., to identify GEP-NEN disease or metastases in a patient previously deemed “negative” by another approach, such as anatomic localization).

The provided methods and compositions may be used for designing, implementing, and monitoring treatment strategies, including patient-specific treatment strategies. In one example, detected expression levels of the GEP-NEN biomarkers serve as surrogate markers for treatment efficacy, e.g., to monitor the effects of surgical therapy, e.g., removal of tumors, targeted medical therapy, e.g., inhibition of tumor secretion/proliferation, and other therapeutic approaches, by detecting remission or recurrence of tumors, even in the form of small micrometastases. The methods also may be used in evaluating clinical symptoms and outcomes, and for histological grading and molecular characterization of GEP-NENs.

The provided biomarkers including GEP-NEN biomarkers, and subsets and panels of the same. Among the provided GEP-NEN biomarkers are gene products, such as DNA, RNA, e.g., transcripts, and protein, which are differentially expressed in GEP-NEN disease, and/or in different stages or sub-types of GEP-NEN, or in different GEP-NEN tumors, such as gene products differentially expressed in metastatic versus non-metastatic tumors, tumors with different degrees of aggressiveness, high versus low-risk tumors, responsive versus non-responsive tumors, tumors exhibiting different pathological classifications and/or likelihood of response to particular courses of treatment, as well as those associated with features of GEP-NEN disease, stage, or type, or with neuroendocrine cells or related cell-types.

For example, the biomarkers include gene products whose expression is associated with or implicated in tumorogenicity, metastasis, or hormone production, or a phenotype of primary or metastatic GEP-NEN, such as adhesion, migration, proliferation, apoptosis, metastasis, and hormone secretion, and those associated with neoplasia or malignancy in general.

Among the biomarkers are GEP-NEN cell secretion products, including hormones and amines, e.g., gastrin, ghrelin, pancreatic polypeptide, substance P, histamine, and serotonin, and growth factors such as tumor growth factor-beta (TGF-β) and connective tissue growth factor (CTGF), which are detectable in the circulation. Secretion products can vary with tumor sub-type and origin.

In one example, the biomarkers are gene products associated with regulatory genotypes (i.e., adhesion, migration, proliferation, apoptosis, metastasis, and/or hormone secretion) that underlay various GEP-NEN subtypes, stages, degrees of aggressiveness, or treatment responsiveness.

A total of 51 differentially expressed biomarker genes have been discovered for the diagnosis, prognosis, and/or monitoring of GEP-NENs. Further details regarding the 51 differentially expressed GEP-NEN biomarkers as well as the housekeeping gene, ALG9, are found in TABLE 1.

TABLE 1
GEP-NEN Biomarker/Houskeeper Sequence Information

| | | | SEQ ID |
|---|---|---|---|
| Gene Name | RefSeq Accession | Sequence | NO: |
| ALG9 | NM_024740.2 | GTCTTTTGTCCCTCGGCGGACACCGTTTGCCAGCCAAAGC | 1 |
| | | TATGTCTGCGCGCTCACCGACTTCATAGGGTGCCGAATTC | |
| | | TTTTTTCCCCAGGCTTGCCATGGCTAGTCGAGGGGCTCGG | |
| | | CAGCGCCTGAAGGGCAGCGGGGCCAGCAGTGGGGATACGG | |
| | | CCCCGGCTGCGGACAAGCTGCGGGAGCTGCTGGGCAGCCG | |
| | | AGAGGCGGGCGGCGCGGAGCACCGGACCGAGTTATCTGGG | |
| | | AACAAAGCAGGACAAGTCTGGGCACCTGAAGGATCTACTG | |
| | | CTTTCAAGTGTCTGCTTTCAGCAAGGTTATGTGCTGCTCT | |
| | | CCTGAGCAACATCTCTGACTGTGATGAAACATTCAACTAC | |
| | | TGGGAGCCAACACACTACCTCATCTATGGGGAAGGGTTTC | |
| | | AGACTTGGGAATATTCCCCAGCATATGCCATTCGCTCCTA | |
| | | TGCTTACCTGTTGCTTCATGCCTGGCCAGCTGCATTTCAT | |
| | | GCAAGAATTCTACAAACTAATAAGATTCTTGTGTTTTACT | |
| | | TTTTGCGATGTCTTCTGGCTTTTGTGAGCTGTATTTGTGA | |
| | | ACTTTACTTTTACAAGGCTGTGTGCAAGAAGTTTGGGTTG | |
| | | CACGTGAGTCGAATGATGCTAGCCTTCTTGGTTCTCAGCA | |
| | | CTGGCATGTTTTGCTCATCATCAGCATTCCTTCCTAGTAG | |
| | | CTTCTGTATGTACACTACGTTGATAGCCATGACTGGATGG | |
| | | TATATGGACAAGACTTCCATTGCTGTGCTGGGAGTAGCAG | |
| | | CTGGGGCTATCTTAGGCTGGCCATTCAGTGCAGCTCTTGG | |
| | | TTTACCCATTGCCTTTGATTTGCTGGTCATGAAACACAGG | |
| | | TGGAAGAGTTTCTTTCATTGGTCGCTGATGGCCCTCATAC | |
| | | TATTTCTGGTGCCTGTGGTGGTCATTGACAGCTACTATTA | |
| | | TGGGAAGTTGGTGATTGCACCACTCAACATTGTTTTGTAT | |
| | | AATGTCTTTACTCCTCATGGACCTGATCTTTATGGTACAG | |
| | | AACCCTGGTATTTCTATTTAATTAATGGATTTCTGAATTT | |
| | | CAATGTAGCCTTTGCTTTGGCTCTCCTAGTCCTACCACTG | |
| | | ACTTCTCTTATGGAATACCTGCTGCAGAGATTTCATGTTC | |
| | | AGAATTTAGGCCACCCGTATTGGCTTACCTTGGCTCCAAT | |
| | | GTATATTTGGTTTATAATTTTCTTCATCCAGCCTCACAAA | |
| | | GAGGAGAGATTTCTTTTCCCTGTGTATCCACTTATATGTC | |
| | | TCTGTGGCGCTGTGGCTCTCTCTGCACTTCAGCACAGTTT | |
| | | TCTGTACTTCCAGAAATGTTACCACTTTGTGTTTCAACGA | |
| | | TATCGCCTGGAGCACTATACTGTGACATCGAATTGGCTGG | |
| | | CATTAGGAACTGTCTTCCTGTTTGGGCTCTTGTCATTTTC | |
| | | TCGCTCTGTGGCACTGTTCAGAGGATATCACGGGCCCCTT | |
| | | GATTTGTATCCAGAATTTTACCGAATTGCTACAGACCCAA | |
| | | CCATCCACACTGTCCCAGAAGGCAGACCTGTGAATGTCTG | |
| | | TGTGGGAAAAGAGTGGTATCGATTTCCCAGCAGCTTCCTT | |
| | | CTTCCTGACAATTGGCAGCTTCAGTTCATTCCATCAGAGT | |
| | | TCAGAGGTCAGTTACCAAAACCTTTTGCAGAAGGACCTCT | |
| | | GGCCACCCGGATTGTTCCTACTGACATGAATGACCAGAAT | |
| | | CTAGAAGAGCCATCCAGATATATTGATATCAGTAAATGCC | |
| | | ATTATTTAGTGGATTTGGACACCATGAGAGAAACACCCCG | |
| | | GGAGCCAAAATATTCATCCAATAAAGAAGAATGGATCAGC | |
| | | TTGGCCTATAGACCATTCCTTGATGCTTCTAGATCTTCAA | |
| | | AGCTGCTGCGGGCATTCTATGTCCCCTTCCTGTCAGATCA | |
| | | GTATACAGTGTACGTAAACTACACCATCCTCAAACCCCGG | |
| | | AAAGCAAAGCAAATCAGGAAGAAAAGTGGAGGTTAGCAAC | |
| | | ACACCTGTGGCCCCAAAGGACAACCATCTTGTTAACTATT | |
| | | GATTCCAGTGACCTGACTCCCTGCAAGTCATCGCCTGTAA | |
| | | CATTTGTAATAAAGGTCTTCTGACATGAATACTGGAATCT | |
| | | GGGTGCTCTGGGCTAGTCAAAGTCTATTTCAAAGTCTAAT | |
| | | CAAAGTCACATTTGCTCCCTGTGTGTGTCTCTGTTCTGCA | |
| | | TGTAAACTTTTTGCAGCTAGGCAGAGAAAGGCCCTAAAGC | |
| | | ACAGATAGATATATTGCTCCACATCTCATTGTTTTTCCTC | |
| | | TGTTCAATTATTTACTAGACCGGAGAAGAGCAGAACCAAC | |
| | | TTACAGGAAGAATTGAAAATCCTGGTACTGGATGGCTGTG | |
| | | ATAAGCTGTTCTCCACACTCTGGCCTGGCATCTGAGAACT | |
| | | AGCAAGCCTCTCTTAGGCCATATGGGCTTCTCCACCAAAG | |
| | | CTGTTTGGCAGCTCCTAGCAGACCTTCTTATTGAAATCCT | |
| | | CATGCTGAAAATGAACACAGCCTAGTTGCCAACCCACATG | |
| | | TCCTTTTCACCTCCAGCAAGACTAAGCTTCTTTAAAGCAC | |
| | | TTCACAGGACTAGGACCCTGTCCTGGAGCTATCTCAGGAA | |
| | | AAAGGTGACCATTTGAGGAACTGTGACCTAATTTTATTAT | |
| | | AATGATGCCTCTAATTTTCATTTCCTTTACAACCAACTGT | |
| | | AACTATAAGGTTGTATTGCTTTTTTGTTCAGTTTTAGCAT | |
| | | GCTATTTTTTGAATTCTAGACTCCTCCATGTGAAGATATC | |
| | | AACAGACAAAACTACAACTGTATAGGACATATTTGGAGAA | |
| | | AATTCTATCAATTGATACATTTGGATGACATCACATTTTT | |
| | | AAGTAATGTAATCTGAGGCCATTGCTGAGGAAATTAAGAA | |
| | | TTTTCCTTTTTTTTTAACCACCCCCAGTGAAAAGGATCAG | |
| | | TGTATATTTATAGCACCTATTTTTTAGTTCTGTCTGTTGT | |
| | | GAGGCACATCCTGCATGGGGCACTTCTAGTCAAATAGGCA | |
| | | ATGATAAGGACCTAATTAAAATGTGATAAGTGTATACTAT | |
| | | TACTTTAAAAGCCTTTACAGTCAGTACTTCAGTTTACAAG | |
| | | GCACTTTCACAGCATCTCGTTTGATCCTCACAGTCACAAC | |
| | | ATGTGGTAGACAAGGCAGGTGATTTTTATCCCCATTTTAC | |
| | | AGATAAGGAAACAGGCTGCGGGTGGGGAGTGAGGGGAGGT | |
| | | AAAGATAGTTAGTTGCCTAAGGTCACACAGCCAGTAAGTA | |
| | | ATAGAGCTGGGACTGGAACCCAGGTTTCCTTACTCTCATC | |
| | | TATTGCTCCTCCATATTCCTCACTCAACCATGAAAACATT | |
| | | ACTTGAAAGGACTGATGAGGTTAACCAGAGACCTAACTGA | |
| | | TATTGTAACTTTCTATTTTAAGGAAGAATTGTGTCTGTAT | |
| | | TTGAGTTCTTTGGAGCCTCCAGTCTGCCTGTGTGTTAGAC | |
| | | CAGCACAGCAGTGCTGTGTGATGCAGCCTGACCTGTGGCA | |
| | | GGAAAGTAGTGCTTCTGTTTGGAAGTCATGTTCTTTTGCA | |
| | | GCCACACAGGATCCAAATATCAGTACTATTCCTGTAGTCA | |
| | | ATCTGGGGTCACATTATAGGTGCCTTATTTCCCTAAGGGT | |
| | | AACTGATCTGAATATCTGCAAATAGGATGAATCTATTTTT | |
| | | CAGAAGTTCCATCTTTCATTTTTCTTTTTTTTTTTGAGAC | |
| | | AGAGTCTCATTCTGTCGCCCATGCTGGAGTGCAGTGGCGC | |
| | | GATCTCGGCTCGCTGCAACCTCTGCCTCCCAGGTTGAAGC | |
| | | AATTCTCATGCCTCAGCCACCCGAGTAGCTGGGATTACAG | |
| | | GCATGCGCCATCATGCCCAGCTAATTTATGTATTTTTAGT | |
| | | AGAGTTGGAGTTTCACCATGTTGGCCAGGCTGGTCTTGGA | |
| | | CTCCTGACCTCAGGTCATCCACCCGCCTCAGCCTCCCAAA | |
| | | GTGCTGGTATTACAGGCGTGAGCCACCGCACCCAGCCCCA | |
| | | TCTTTCATTTTCAAAGAGAAGGGCATTCTAATAGGAACTG | |
| | | GTGCCAAGAGAGAAGAAAAGAAGTGATAACAGAAGAAATG | |
| | | GCTAGTTACAATATTAAAAAGCTCCTCTTTGAGATCTCCT | |
| | | CTGCAGGAATATCAGAGACGGAGTTGAAGCGCTGGAGAGG | |
| | | TAATAGGTCTAGACAGTACAGAACAATAACTGGGGAGTGT | |
| | | GTGAGGATAGACTGGGCTCCCCCTTGCTTGAAAGATCTCT | |
| | | GGCATTTAATTCTCAATTCTTGATTACTATTTTCCAGTGT | |
| | | AAAACTAGCACATATGATCTGACTACAGGACAGAGAATTT | |
| | | TAAGTGAAACATTTGCCTTACTTGCAGTAATAATGTGCTG | |
| | | TTCTTCACAGTAGCTAAGGCCCTCTATGTTTCCCAGAGGT | |
| | | AAATAAGAATCCAGGAATGGAGGTCCATCTGTGATGAATG | |
| | | GCTTTTTTCTAATCAAAGTAGTATAATGCTGTTTTATCTG | |
| | | TTTTGTCATCTTGTTTTTTTTTTTTTTTAAAAAAACAAAA | |
| | | CCTTAATTATAATATAGCGCAAAGAAAGGCCAGGACTGAT | |
| | | GCAGGGATTCCTTGGAAATATCAGTTCCTATCACTTTTAA | |
| | | AACCTGATTTTGGATCTCTCTGTTCTATGTATGTCTTTAG | |
| | | TGAGAGCACAATACATGGCAGAACGCTGTGCCAAATGTTA | |
| | | TAGGTAAGGAATATAGAAATGAATGTTTTTTGTTGTGAAG | |
| | | GTGTTTTCATGTGATATTTTATAAACACATTTTAAAAAAT | |
| | | CTCCATCACTTTTTAGTATAGGAAGGATAGCTTTGCCTGG | |
| | | GAAAAACAGTTTCAACACACCTGCTCAGAGTAGCAGTTCT | |
| | | CCCTCAAAAAAGCAGTGTTCAGCCTGCACTGACTGTTCTG | |
| | | CTTGCCAAAAGGAGGAAGCATGCAAGATACTTATTTCTCC | |
| | | ATAGATTGTGGAGTATAGAGGGATGTGGGACTACAGATTA | |
| | | TTATTTTTTTTCCCCGAGACAGAGTCTTGCTCTGTCGCCC | |
| | | AGGTTGGAACACAATGGCACGACCTCAGCTCACTGCAACC | |
| | | TCTGTCTCCCGGGTTCAAGCAATTCTCCTGCTTCAGCCTC | |
| | | CTGAGTAGCTGGGATTACAGGCACACACCACCACCGCACT | |
| | | CAGCTAATTTTTGTATTTTTAGTAGAGGTGGGGTTTTACC | |
| | | ATGTTGGCCAGGCTGGTCTTAAACTCCTGACCTTGTAATC | |
| | | ATCCCGCCTCGGCCTCCTAAAGTGCTAGGATTACAGGCAT | |
| | | GAGCCACCGCACCCGGCCCAGATAATTTTTAATAGCCTTT | |
| | | GATCATGGGGTGAGTGAGGGAGTAGGTATACTTGGCAAAT | |
| | | GCATGGTTCTCTGATTTCTAGCTCTAAAGCAGCCTTATCT | |
| | | GAATCCCCAAATCTTGTGATGCTGAGTACCATTACTGAAC | |
| | | CAGTCTGCACGGTAGGCATCTGCTACCAAAATTTACCTCC | |
| | | TACCTGGTAGGTGTCATCTGATAAGAAAGAAGACAGGTTA | |
| | | TTTTAATTTTTTGAGATAATCACAGAAAATTGCAGCCCAT | |
| | | ACTCTTTATTACCGAATTCAAGTTTGGAAATAGACCCTTT | |
| | | GTTTTAAATCATGATGGGTCTTTATCCCAATCATTTATCT | |
| | | GGGTCATTTTTCCAACTTTGGAGTTCTAGGAAAGAACCTT | |
| | | GAAAACCTGATATGATTCTGCAGCATGAGGTCTACGGTGA | |
| | | CCATTTGGGCAAAGCTCCAGTGGCAATCATTTATTGTGTT | |
| | | TTGCATTTCCTGGGATTTATTGAAATAAGAATTCACTGTG | |
| | | ATTATGTAGTCTTCTGGCTAGTATCAGGCAGCTCTGCTTT | |
| | | TAATTTGGTTAATTTTATTTTCTCTGAAGAGGGAGAAGAG | |
| | | GTACAATTTAATCTTGGCCTCCACAAGCATATTAAAGCTC | |
| | | ACGTGTTAATCAGTGCATTCTTATGCTCCTACATTAAATG | |
| | | CCTTGGGTAAATGGATAAATGGACATGTGCCCAGCTTTAA | |
| | | TTTTTTTTGCAACAGAAAGATCAGACTTCCGTATGGCATC | |
| | | GTTGGATTTCAGAGGCTTTCTGGTGTATCTGTAAATCTGA | |
| | | ATGTTGCCTTCTGCCAGTCTGTATAACCAGGTGATTCATG | |
| | | CTGCAAATGAAATCAGGAAGCAGTAAAGTGTTAAAGCAAG | |
| | | AGTATTGTCCAATTCACTTGTCTTCCTGATCCTTGTACTT | |
| | | TATTTCACGTGTCGGTGTTTACATTACATACTTATATTTC | |
| | | CTGTGAAAGAAAGAGTTAAATAAATTGTAGCAGTTTGA | |
| AKAP8L | NM_014371.3 | ACTGATATGAGGAGGCATAGAGATAGACAGCGGTTCCTTC | 2 |
| | | CAATAGACGTGAAGCCGAGGCCGGTATGAGCCAATGCGGT | |
| | | CGGGAGGCGGGGCTCGGGTGTGTGTGGAGGGGACCCTGTG | |
| | | GTTAGCAGCAGCTATCGCAGCGTCGGATGTTCAGAGCAGC | |
| | | AGAAGCCGGCGTCGTCGGATGTTGTGTTGCCCGCCACCAT | |
| | | GAGCTACACAGGCTTTGTCCAGGGATCTGAAACCACTTTG | |
| | | CAGTCGACATACTCGGATACCAGCGCTCAGCCCACCTGTG | |
| | | ATTATGGATATGGAACTTGGAACTCTGGGACAAATAGAGG | |
| | | CTACGAGGGCTATGGCTATGGCTATGGCTATGGCCAGGAT | |
| | | AACACCACCAACTATGGGTATGGTATGGCCACTTCACACT | |
| | | CTTGGGAAATGCCTAGCTCTGACACAAATGCAAACACTAG | |
| | | TGCCTCGGGTAGCGCCAGTGCCGATTCCGTTTTATCCAGA | |
| | | ATTAACCAGCGCTTAGATATGGTGCCGCATTTGGAGACAG | |
| | | ACATGATGCAAGGAGGCGTGTACGGCTCAGGTGGAGAAAG | |
| | | GTATGACTCTTATGAGTCCTGCGACTCGAGGGCCGTCCTG | |
| | | AGTGAGCGCGACCTGTACCGGTCAGGCTATGACTACAGCG | |
| | | AGCTTGACCCTGAGATGGAAATGGCCTATGAGGGCCAATA | |
| | | CGATGCCTACCGCGACCAGTTCCGCATGCGTGGCAACGAC | |
| | | ACCTTCGGTCCCAGGGCACAGGGCTGGGCCCGGGATGCCC | |
| | | GGAGCGGCCGGCCAATGGCCTCAGGCTATGGGCGCATGTG | |
| | | GGAAGACCCCATGGGGGCCCGGGGCCAGTGCATGTCTGGT | |
| | | GCCTCTCGGCTGCCCTCCCTCTTCTCCCAGAACATCATCC | |
| | | CCGAGTACGGCATGTTCCAGGGCATGCGAGGTGGGGGCGC | |
| | | CTTCCCGGGCGGCTCCCGCTTTGGTTTCGGGTTTGGCAAT | |
| | | GGCATGAAGCAGATGAGGCGGACCTGGAAGACCTGGACCA | |
| | | CAGCCGACTTCCGAACCAAGAAGAAGAAGAGAAAGCAGGG | |
| | | CGGCAGTCCTGATGAGCCAGATAGCAAAGCCACCCGCACG | |
| | | GACTGCTCGGACAACAGCGACTCAGACAATGATGAGGGCA | |
| | | CCGAGGGGGAAGCCACAGAGGGCCTTGAAGGCACCGAGGC | |
| | | TGTGGAGAAGGGCTCCAGAGTGGACGGAGAGGATGAGGAG | |
| | | GGAAAAGAGGATGGGAGAGAAGAAGGCAAAGAGGATCCAG | |
| | | AGAAGGGGGCCCTAACCACCCAGGATGAAAATGGCCAGAC | |
| | | CAAGCGCAAGTTGCAGGCAGGCAAGAAGAGTCAGGACAAG | |
| | | CAGAAAAAGCGGCAGCGAGACCGCATGGTGGAAAGGATCC | |
| | | AGTTTGTGTGTTCTCTGTGCAAATACCGGACCTTCTATGA | |
| | | GGACGAGATGGCCAGCCATCTTGACAGCAAGTTCCACAAG | |
| | | GAACACTTTAAGTACGTAGGCACCAAGCTCCCTAAGCAGA | |
| | | CGGCTGACTTTCTGCAGGAGTACGTCACTAACAAGACCAA | |
| | | GAAGACAGAGGAGCTCCGAAAAACCGTGGAGGACCTTGAT | |
| | | GGCCTCATCCAGCAAATCTACAGAGACCAGGATCTGACCC | |
| | | AGGAAATTGCCATGGAGCATTTTGTGAAGAAGGTGGAGGC | |
| | | AGCCCATTGTGCAGCCTGCGACCTCTTCATTCCCATGCAG | |
| | | TTTGGGATCATCCAGAAGCATCTGAAGACCATGGATCACA | |
| | | ACCGGAACCGCAGGCTCATGATGGAGCAGTCCAAGAAGTC | |
| | | CTCCCTCATGGTGGCCCGCAGTATTCTCAACAACAAGCTC | |
| | | ATCAGCAAGAAGCTGGAGCGCTACCTGAAGGGCGAGAACC | |
| | | CTTTCACCGACAGCCCCGAGGAGGAGAAGGAGCAGGAGGA | |
| | | GGCTGAGGGCGGTGCCCTGGACGAGGGGGCGCAGGGCGAA | |
| | | GCGGCAGGGATCTCGGAGGGCGCAGAGGGCGTGCCGGCGC | |
| | | AGCCTCCCGTGCCCCCAGAGCCAGCCCCCGGGGCCGTGTC | |
| | | GCCGCCACCGCCGCCGCCCCCAGAGGAGGAGGAGGAGGGC | |
| | | GCCGTGCCCTTGCTGGGAGGGGCGCTGCAACGCCAGATCC | |
| | | GCGGCATCCCGGGCCTCGACGTGGAGGACGACGAGGAGGG | |
| | | CGGCGGGGGCGCCCCGTGACCCGAGCTCGGGGCGGGCGGA | |
| | | GCCCGCGTGGCCGAAGCTGGAAACCAAACCTAATAAAGTT | |
| | | TTCCCATCCCACCAAAAAAAAAAAAAAAAAA | |
| APLP2 | NM_001142276.1 | AGAAGGAGGGCGTGGTAATATGAAGTCAGTTCCGGTTGGT | 3 |
| | | GTAAAACCCCCGGGGCGGCGGCGAACTGGCTTTAGATGCT | |
| | | TCTGGGTCGCGGTGTGCTAAGCGAGGAGTCCGAGTGTGTG | |
| | | AGCTTGAGAGCCGCGCGCTAGAGCGACCCGGCGAGGGATG | |
| | | GCGGCCACCGGGACCGCGGCCGCCGCAGCCACGGGCAGGC | |
| | | TCCTGCTTCTGCTGCTGGTGGGGCTCACGGCGCCTGCCTT | |
| | | GGCGCTGGCCGGCTACATCGAGGCTCTTGCAGCCAATGCC | |
| | | GGAACAGGATTTGCTGTTGCTGAGCCTCAAATCGCAATGT | |
| | | TTTGTGGGAAGTTAAATATGCATGTGAACATTCAGACTGG | |
| | | GAAATGGGAACCTGATCCAACAGGCACCAAGAGCTGCTTT | |
| | | GAAACAAAAGAAGAAGTTCTTCAGTACTGTCAGGAGATGT | |
| | | ATCCAGAGCTACAGATCACAAATGTGATGGAGGCAAACCA | |
| | | GCGGGTTAGTATTGACAACTGGTGCCGGAGGGACAAAAAG | |
| | | CAATGCAAGAGTCGCTTTGTTACACCTTTCAAGTGTCTCG | |
| | | TGGGTGAATTTGTAAGTGATGTCCTGCTAGTTCCAGAAAA | |
| | | GTGCCAGTTTTTCCACAAAGAGCGGATGGAGGTGTGTGAG | |
| | | AATCACCAGCACTGGCACACGGTAGTCAAAGAGGCATGTC | |
| | | TGACTCAGGGAATGACCTTATATAGCTACGGCATGCTGCT | |
| | | CCCATGTGGGGTAGACCAGTTCCATGGCACTGAATATGTG | |
| | | TGCTGCCCTCAGACAAAGATTATTGGATCTGTGTCAAAAG | |
| | | AAGAGGAAGAGGAAGATGAAGAGGAAGAGGAAGAGGAAGA | |
| | | TGAAGAGGAAGACTATGATGTTTATAAAAGTGAATTTCCT | |
| | | ACTGAAGCAGATCTGGAAGACTTCACAGAAGCAGCTGTGG | |
| | | ATGAGGATGATGAGGATGAGGAAGAAGGGGAGGAAGTGGT | |
| | | GGAGGACCGAGATTACTACTATGACACCTTCAAAGGAGAT | |
| | | GACTACAATGAGGAGAATCCTACTGAACCCGGCAGCGACG | |
| | | GCACCATGTCAGACAAGGAAATTACTCATGATGTCAAAGC | |
| | | TGTCTGCTCCCAGGAGGCGATGACGGGGCCCTGCCGGGCC | |
| | | GTGATGCCTCGTTGGTACTTCGACCTCTCCAAGGGAAAGT | |
| | | GCGTGCGCTTTATATATGGTGGCTGCGGCGGCAACAGGAA | |
| | | CAATTTTGAGTCTGAGGATTATTGTATGGCTGTGTGTAAA | |
| | | GCGATGATTCCTCCAACTCCTCTGCCAACCAATGATGTTG | |
| | | ATGTGTATTTCGAGACCTCTGCAGATGATAATGAGCATGC | |
| | | TCGCTTCCAGAAGGCTAAGGAGCAGCTGGAGATTCGGCAC | |
| | | CGCAACCGAATGGACAGGGTAAAGAAGGAATGGGAAGAGG | |
| | | CAGAGCTTCAAGCTAAGAACCTCCCCAAAGCAGAGAGGCA | |
| | | GACTCTGATTCAGCACTTCCAAGCCATGGTTAAAGCTTTA | |
| | | GAGAAGGAAGCAGCCAGTGAGAAGCAGCAGCTGGTGGAGA | |
| | | CCCACCTGGCCCGAGTGGAAGCTATGCTGAATGACCGCCG | |
| | | TCGGATGGCTCTGGAGAACTACCTGGCTGCCTTGCAGTCT | |
| | | GACCCGCCACGGCCTCATCGCATTCTCCAGGCCTTACGGC | |
| | | GTTATGTCCGTGCTGAGAACAAAGATCGCTTACATACCAT | |
| | | CCGTCATTACCAGCATGTGTTGGCTGTTGACCCAGAAAAG | |
| | | GCGGCCCAGATGAAATCCCAGGTGATGACACATCTCCACG | |
| | | TGATTGAAGAAAGGAGGAACCAAAGCCTCTCTCTGCTCTA | |
| | | CAAAGTACCTTATGTAGCCCAAGAAATTCAAGAGGAAATT | |
| | | GATGAGCTCCTTCAGGAGCAGCGTGCAGATATGGACCAGT | |
| | | TCACTGCCTCAATCTCAGAGACCCCTGTGGACGTCCGGGT | |
| | | GAGCTCTGAGGAGAGTGAGGAGATCCCACCGTTCCACCCC | |
| | | TTCCACCCCTTCCCAGCCCTACCTGAGAACGAAGGATCTG | |
| | | GAGTGGGAGAGCAGGATGGGGGACTGATCGGTGCCGAAGA | |
| | | GAAAGTGATTAACAGTAAGAATAAAGTGGATGAAAACATG | |
| | | GTCATTGACGAGACTCTGGATGTTAAGGAAATGATTTTCA | |
| | | ATGCCGAGAGAGTTGGAGGCCTCGAGGAAGAGCGGGAATC | |
| | | CGTGGGCCCACTGCGGGAGGACTTCAGTCTGAGTAGCAGT | |
| | | GCTCTCATTGGCCTGCTGGTCATCGCAGTGGCCATTGCCA | |
| | | CGGTCATCGTCATCAGCCTGGTGATGCTGAGGAAGAGGCA | |
| | | GTATGGCACCATCAGCCACGGGATCGTGGAGGTTGATCCA | |
| | | ATGCTCACCCCAGAAGAGCGTCACCTGAACAAGATGCAGA | |
| | | ACCATGGCTATGAGAACCCCACCTACAAATACCTGGAGCA | |
| | | GATGCAGATTTAGGTGGCAGGGAGCGCGGCAGCCCTGGCG | |
| | | GAGGGATGCAGGTGGGCCGGAAGATCCCACGATTCCGATC | |
| | | GACTGCCAAGCAGCAGCCGCTGCCAGGGGCTGCGTCTGAC | |
| | | ATCCTGACCTCCTGGACTGTAGGACTATATAAAGTACTAC | |
| | | TGTAGAACTGCAATTTCCATTCTTTTAAATGGGTGAAAAA | |
| | | TGGTAATATAACAATATATGATATATAAACCTTAAATGAA | |
| | | AAAAATGATCTATTGCAGATATTTGATGTAGTTTTCTTTT | |
| | | TTAAATTAATCAGAAACCCCACTTCCATTGTATTGTCTGA | |
| | | CACATGCTCTCAATATATAATAAATGGGAAATGTCGATTT | |
| | | TCAATAATAGACTTATATGCAGGCTGTCGTTCCGGTTATG | |
| | | TTGTGTAAGTCAACTCTTCAGCCTCATTCACTGTCCTGGC | |
| | | TTTTATTTAAAGAAAAAAAAGGCAGTATTCCCTTTTTAAA | |
| | | TGAGCTTTCAGGAAGTTGCTGAGAAATGGGGTGGAATAGG | |
| | | GAACTGTAATGGCCACTGAAGCACGTGAGAGACCCTCGCA | |
| | | AAATGATGTGAAAGGACCAGTTTCTTGAAGTCCAGTGTTT | |
| | | CCACGGCTGGATACCTGTGTGTCTCCATAAAAGTCCTGTC | |
| | | ACCAAGGACGTTAAAGGCATTTTATTCCAGCGTCTTCTAG | |
| | | AGAGCTTAGTGTATACAGATGAGGGTGTCCGCTGCTGCTT | |
| | | TCCTTCGGAATCCAGTGCTTCCACAGAGATTAGCCTGTAG | |
| | | CTTATATTTGACATTCTTCACTGTCTGTTGTTTACCTACC | |
| | | GTAGCTTTTTACCGTTCACTTCCCCTTCCAACTATGTCCA | |
| | | GATGTGCAGGCTCCTCCTCTCTGGACTTTCTCCAAAGGCA | |
| | | CTGACCCTCGGCCTCTACTTTGTCCCCTCACCTCCACCCC | |
| | | CTCCTGTCACCGGCCTTGTGACATTCACTCAGAGAAGACC | |
| | | ACACCAAGGAGGCGGCCGCTGGCCCAGGAGAGAACACGGG | |
| | | GAGGTTTGTTTGTGTGAAAGGAAAGTAGTCCAGGCTGTCC | |
| | | CTGAAACTGAGTCTGTGGACACTGTGGAAAGCTTTGAACA | |
| | | ATTGTGTTTTCGTCACAGGAGTCTTTGTAATGCTTGTACA | |
| | | GTTGATGTCGATGCTCACTGCTTCTGCTTTTTCTTTCTTT | |
| | | TTATTTTAAATCTGAAGGTTCTGGTAACCTGTGGTGTATT | |
| | | TTTATTTTCCTGTGACTGTTTTTGTTTTGTTTTTTTCCTT | |
| | | TTTCCTCCCCTTTGACCCTATTCATGTCTCTACCCACTAT | |
| | | GCACAGATTAAACTTCACCTACAAACTCCTTAATATGATC | |
| | | TGTGGAGAATGTACACAGTTTAAACACATCAATAAATACT | |
| | | TTAACTTCCACCGAGAAAAAAAAAAAAAAAA | |
| ARAF1 | NM_001654.4 | CTTGACAGACGTGACCCTGACCCAATAAGGGTGGAAGGCT | 4 |
| | | GAGTCCCGCAGAGCCAATAACGAGAGTCCGAGAGGCGACG | |
| | | GAGGCGGACTCTGTGAGGAAACAAGAAGAGAGGCCCAAGA | |
| | | TGGAGACGGCGGCGGCTGTAGCGGCGTGACAGGAGCCCCA | |
| | | TGGCACCTGCCCAGCCCCACCTCAGCCCATCTTGACAAAA | |
| | | TCTAAGGCTCCATGGAGCCACCACGGGGCCCCCCTGCCAA | |
| | | TGGGGCCGAGCCATCCCGGGCAGTGGGCACCGTCAAAGTA | |
| | | TACCTGCCCAACAAGCAACGCACGGTGGTGACTGTCCGGG | |
| | | ATGGCATGAGTGTCTACGACTCTCTAGACAAGGCCCTGAA | |
| | | GGTGCGGGGTCTAAATCAGGACTGCTGTGTGGTCTACCGA | |
| | | CTCATCAAGGGACGAAAGACGGTCACTGCCTGGGACACAG | |
| | | CCATTGCTCCCCTGGATGGCGAGGAGCTCATTGTCGAGGT | |
| | | CCTTGAAGATGTCCCGCTGACCATGCACAATTTTGTACGG | |
| | | AAGACCTTCTTCAGCCTGGCGTTCTGTGACTTCTGCCTTA | |
| | | AGTTTCTGTTCCATGGCTTCCGTTGCCAAACCTGTGGCTA | |
| | | CAAGTTCCACCAGCATTGTTCCTCCAAGGTCCCCACAGTC | |
| | | TGTGTTGACATGAGTACCAACCGCCAACAGTTCTACCACA | |
| | | GTGTCCAGGATTTGTCCGGAGGCTCCAGACAGCATGAGGC | |
| | | TCCCTCGAACCGCCCCCTGAATGAGTTGCTAACCCCCCAG | |
| | | GGTCCCAGCCCCCGCACCCAGCACTGTGACCCGGAGCACT | |
| | | TCCCCTTCCCTGCCCCAGCCAATGCCCCCCTACAGCGCAT | |
| | | CCGCTCCACGTCCACTCCCAACGTCCATATGGTCAGCACC | |
| | | ACGGCCCCCATGGACTCCAACCTCATCCAGCTCACTGGCC | |
| | | AGAGTTTCAGCACTGATGCTGCCGGTAGTAGAGGAGGTAG | |
| | | TGATGGAACCCCCCGGGGGAGCCCCAGCCCAGCCAGCGTG | |
| | | TCCTCGGGGAGGAAGTCCCCACATTCCAAGTCACCAGCAG | |
| | | AGCAGCGCGAGCGGAAGTCCTTGGCCGATGACAAGAAGAA | |
| | | AGTGAAGAACCTGGGGTACCGGGACTCAGGCTATTACTGG | |
| | | GAGGTACCACCCAGTGAGGTGCAGCTGCTGAAGAGGATCG | |
| | | GGACGGGCTCGTTTGGCACCGTGTTTCGAGGGCGGTGGCA | |
| | | TGGCGATGTGGCCGTGAAGGTGCTCAAGGTGTCCCAGCCC | |
| | | ACAGCTGAGCAGGCCCAGGCTTTCAAGAATGAGATGCAGG | |
| | | TGCTCAGGAAGACGCGACATGTCAACATCTTGCTGTTTAT | |
| | | GGGCTTCATGACCCGGCCGGGATTTGCCATCATCACACAG | |
| | | TGGTGTGAGGGCTCCAGCCTCTACCATCACCTGCATGTGG | |
| | | CCGACACACGCTTCGACATGGTCCAGCTCATCGACGTGGC | |
| | | CCGGCAGACTGCCCAGGGCATGGACTACCTCCATGCCAAG | |
| | | AACATCATCCACCGAGATCTCAAGTCTAACAACATCTTCC | |
| | | TACATGAGGGGCTCACGGTGAAGATCGGTGACTTTGGCTT | |
| | | GGCCACAGTGAAGACTCGATGGAGCGGGGCCCAGCCCTTG | |
| | | GAGCAGCCCTCAGGATCTGTGCTGTGGATGGCAGCTGAGG | |
| | | TGATCCGTATGCAGGACCCGAACCCCTACAGCTTCCAGTC | |
| | | AGACGTCTATGCCTACGGGGTTGTGCTCTACGAGCTTATG | |
| | | ACTGGCTCACTGCCTTACAGCCACATTGGCTGCCGTGACC | |
| | | AGATTATCTTTATGGTGGGCCGTGGCTATCTGTCCCCGGA | |
| | | CCTCAGCAAAATCTCCAGCAACTGCCCCAAGGCCATGCGG | |
| | | CGCCTGCTGTCTGACTGCCTCAAGTTCCAGCGGGAGGAGC | |
| | | GGCCCCTCTTCCCCCAGATCCTGGCCACAATTGAGCTGCT | |
| | | GCAACGGTCACTCCCCAAGATTGAGCGGAGTGCCTCGGAA | |
| | | CCCTCCTTGCACCGCACCCAGGCCGATGAGTTGCCTGCCT | |
| | | GCCTACTCAGCGCAGCCCGCCTTGTGCCTTAGGCCCCGCC | |
| | | CAAGCCACCAGGGAGCCAATCTCAGCCCTCCACGCCAAGG | |
| | | AGCCTTGCCCACCAGCCAATCAATGTTCGTCTCTGCCCTG | |
| | | ATGCTGCCTCAGGATCCCCCATTCCCCACCCTGGGAGATG | |
| | | AGGGGGTCCCCATGTGCTTTTCCAGTTCTTCTGGAATTGG | |
| | | GGGACCCCCGCCAAAGACTGAGCCCCCTGTCTCCTCCATC | |
| | | ATTTGGTTTCCTCTTGGCTTTGGGGATACTTCTAAATTTT | |
| | | GGGAGCTCCTCCATCTCCAATGGCTGGGATTTGTGGCAGG | |
| | | GATTCCACTCAGAACCTCTCTGGAATTTGTGCCTGATGTG | |
| | | CCTTCCACTGGATTTTGGGGTTCCCAGCACCCCATGTGGA | |
| | | TTTTGGGGGGTCCCTTTTGTGTCTCCCCCGCCATTCAAGG | |
| | | ACTCCTCTCTTTCTTCACCAAGAAGCACAGAATTCTGCTG | |
| | | GGCCTTTGCTTGTTTAAAAAAAAAAAAAAAAAAAAAAAAA | |
| | | AAAAAAAAAAAAAAAAAAAAAAAAAAAAAAAAAAAAAAAA | |
| | | AA | |
| ATP6V1H | NM_015941.3 | AGCAGTCACGTGCCTCCGATCACGTGACCGGCGCCTCTGT | 5 |
| | | CATTCTACTGCGGCCGCCCTGGCTTCCTTCTACCTGTGCG | |
| | | GCCCTCAACGTCTCCTTGGTGCGGGACCCGCTTCACTTTC | |
| | | GGCTCCCGGAGTCTCCCTCCACTGCTCAGACCTCTGGACC | |
| | | TGACAGGAGACGCCTACTTGGCTCTGACGCGGCGCCCCAG | |
| | | CCCGGCTGTGTCCCCGGCGCCCCGGACCACCCTCCCTGCC | |
| | | GGCTTTGGGTGCGTTGTGGGGTCCCGAGGATTCGCGAGAT | |
| | | TTGTTGAAAGACATTCAAGATTACGAAGTTTAGATGACCA | |
| | | AAATGGATATCCGAGGTGCTGTGGATGCTGCTGTCCCCAC | |
| | | CAATATTATTGCTGCCAAGGCTGCAGAAGTTCGTGCAAAC | |
| | | AAAGTCAACTGGCAATCCTATCTTCAGGGACAGATGATTT | |
| | | CTGCTGAAGATTGTGAGTTTATTCAGAGGTTTGAAATGAA | |
| | | ACGAAGCCCTGAAGAGAAGCAAGAGATGCTTCAAACTGAA | |
| | | GGCAGCCAGTGTGCTAAAACATTTATAAATCTGATGACTC | |
| | | ATATCTGCAAAGAACAGACCGTTCAGTATATACTAACTAT | |
| | | GGTGGATGATATGCTGCAGGAAAATCATCAGCGTGTTAGC | |
| | | ATTTTCTTTGACTATGCAAGATGTAGCAAGAACACTGCGT | |
| | | GGCCCTACTTTCTGCCAATGTTGAATCGCCAGGATCCCTT | |
| | | CACTGTTCATATGGCAGCAAGAATTATTGCCAAGTTAGCA | |
| | | GCTTGGGGAAAAGAACTGATGGAAGGCAGTGACTTAAATT | |
| | | ACTATTTCAATTGGATAAAAACTCAGCTGAGTTCACAGAA | |
| | | ACTGCGTGGTAGCGGTGTTGCTGTTGAAACAGGAACAGTC | |
| | | TCTTCAAGTGATAGTTCGCAGTATGTGCAGTGCGTGGCCG | |
| | | GGTGTTTGCAGCTGATGCTCCGGGTCAATGAGTACCGCTT | |
| | | TGCTTGGGTGGAAGCAGATGGGGTAAATTGCATAATGGGA | |
| | | GTGTTGAGTAACAAGTGTGGCTTTCAGCTCCAGTATCAAA | |
| | | TGATTTTTTCAATATGGCTCCTGGCATTCAGTCCTCAAAT | |
| | | GTGTGAACACCTGCGGCGCTATAATATCATTCCAGTTCTG | |
| | | TCTGATATCCTTCAGGAGTCTGTCAAAGAGAAAGTAACAA | |
| | | GAATCATTCTTGCAGCATTTCGTAACTTTTTAGAAAAATC | |
| | | AACTGAAAGAGAAACTCGCCAAGAATATGCCCTGGCTATG | |
| | | ATTCAGTGCAAAGTTCTGAAACAGTTGGAGAACTTGGAAC | |
| | | AGCAGAAGTACGATGATGAAGATATCAGCGAAGATATCAA | |
| | | ATTTCTTTTGGAAAAACTTGGAGAGAGTGTCCAGGACCTT | |
| | | AGTTCATTTGATGAATACAGTTCAGAACTTAAATCTGGAA | |
| | | GGTTGGAATGGAGTCCTGTGCACAAATCTGAGAAATTTTG | |
| | | GAGAGAGAATGCTGTGAGGTTAAATGAGAAGAATTATGAA | |
| | | CTCTTGAAAATCTTGACAAAACTTTTGGAAGTGTCAGATG | |
| | | ATCCCCAAGTCTTAGCTGTTGCTGCTCACGATGTTGGAGA | |
| | | ATATGTGCGGCATTATCCACGAGGCAAACGGGTCATCGAG | |
| | | CAGCTCGGTGGGAAGCAGCTGGTCATGAACCACATGCATC | |
| | | ATGAAGACCAGCAGGTCCGCTATAATGCTCTGCTGGCCGT | |
| | | GCAGAAGCTCATGGTGCACAACTGGGAATACCTTGGCAAG | |
| | | CAGCTCCAGTCCGAGCAGCCCCAGACCGCTGCCGCCCGAA | |
| | | GCTAAGCCTGCCTCTGGCCTTCCCCTCCGCCTCAATGCAG | |
| | | AACCAGTAGTGGGAGCACTGTGTTTAGAGTTAAGAGTGAA | |
| | | CACTGTTTGATTTTACTTGGAATTTCCTCTGTTATATAGC | |
| | | TTTTCCCAATGCTAATTTCCAAACAACAACAACAAAATAA | |
| | | CATGTTTGCCTGTTAAGTTGTATAAAAGTAGGTGATTCTG | |
| | | TATTTAAAGAAAATATTACTGTTACATATACTGCTTGCAA | |
| | | TTTCTGTATTTATTGTTCTCTGGAAATAAATATAGTTATT | |
| | | AAAGGATTCTCACTCCAAACATGGCCTCTCTCTTTACTTG | |
| | | GACTTTGAACAAAAGTCAACTGTTGTCTCTTTTCAAACCA | |
| | | AATTGGGAGAATTGTTGCAAAGTAGTGAATGGCAAATAAA | |
| | | TGTTTTAAAATCTATCGCTCTATCAA | |
| BNIP3L | NM_004331.2 | CGTCAGGGGCAGGGGAGGGACGGCGCAGGCGCAGAAAAGG | 6 |
| | | GGGCGGCGGACTCGGCTTGTTGTGTTGCTGCCTGAGTGCC | |
| | | GGAGACGGTCCTGCTGCTGCCGCAGTCCTGCCAGCTGTCC | |
| | | GACAATGTCGTCCCACCTAGTCGAGCCGCCGCCGCCCCTG | |
| | | CACAACAACAACAACAACTGCGAGGAAAATGAGCAGTCTC | |
| | | TGCCCCCGCCGGCCGGCCTCAACAGTTCCTGGGTGGAGCT | |
| | | ACCCATGAACAGCAGCAATGGCAATGATAATGGCAATGGG | |
| | | AAAAATGGGGGGCTGGAACACGTACCATCCTCATCCTCCA | |
| | | TCCACAATGGAGACATGGAGAAGATTCTTTTGGATGCACA | |
| | | ACATGAATCAGGACAGAGTAGTTCCAGAGGCAGTTCTCAC | |
| | | TGTGACAGCCCTTCGCCACAAGAAGATGGGCAGATCATGT | |
| | | TTGATGTGGAAATGCACACCAGCAGGGACCATAGCTCTCA | |
| | | GTCAGAAGAAGAAGTTGTAGAAGGAGAGAAGGAAGTCGAG | |
| | | GCTTTGAAGAAAAGTGCGGACTGGGTATCAGACTGGTCCA | |
| | | GTAGACCCGAAAACATTCCACCCAAGGAGTTCCACTTCAG | |
| | | ACACCCTAAACGTTCTGTGTCTTTAAGCATGAGGAAAAGT | |
| | | GGAGCCATGAAGAAAGGGGGTATTTTCTCCGCAGAATTTC | |
| | | TGAAGGTGTTCATTCCATCTCTCTTCCTTTCTCATGTTTT | |
| | | GGCTTTGGGGCTAGGCATCTATATTGGAAAGCGACTGAGC | |
| | | ACACCCTCTGCCAGCACCTACTGAGGGAAAGGAAAAGCCC | |
| | | CTGGAAATGCGTGTGACCTGTGAAGTGGTGTATTGTCACA | |
| | | GTAGCTTATTTGAACTTGAGACCATTGTAAGCATGACCCA | |
| | | ACCTACCACCCTGTTTTTACATATCCAATTCCAGTAACTC | |
| | | TCAAATTCAATATTTTATTCAAACTCTGTTGAGGCATTTT | |
| | | ACTAACCTTATACCCTTTTTGGCCTGAAGACATTTTAGAA | |
| | | TTTCCTAACAGAGTTTACTGTTGTTTAGAAATTTGCAAGG | |
| | | GCTTCTTTTCCGCAAATGCCACCAGCAGATTATAATTTTG | |
| | | TCAGCAATGCTATTATCTCTAATTAGTGCCACCAGACTAG | |
| | | ACCTGTATCATTCATGGTATAAATTTTACTCTTGCAACAT | |
| | | AACTACCATCTCTCTCTTAAAACGAGATCAGGTTAGCAAA | |
| | | TGATGTAAAAGAAGCTTTATTGTCTAGTTGTTTTTTTTCC | |
| | | CCCAAGACAAAGGCAAGTTTCCCTAAGTTTGAGTTGATAG | |
| | | TTATTAAAAAGAAAACAAAACAAAAAAAAAAGGCAAGGCA | |
| | | CAACAAAAAAATATCCTGGGCAATAAAAAAAATATTTTAA | |
| | | ACCAGCTTTGGAGCCACTTTTTTGTCTAAGCCTCCTAATA | |
| | | GCGTCTTTTAATTTATAGGAGGCAAACTGTATAAATGATA | |
| | | GGTATGAAATAGAATAAGAAGTAAAATACATCAGCAGATT | |
| | | TTCATACTAGTATGTTGTAATGCTGTCTTTTCTATGGTGT | |
| | | AGAATCTTTCTTTCTGATAAGGAACGTCTCAGGCTTAGAA | |
| | | ATATATGAAATTGCTTTTTGAGATTTTTGCGTGTGTGTTT | |
| | | GATATTTTTTACGATAATTAGCTGCATGTGAATTTTTCAT | |
| | | GACCTTCTTTACATTTTTTATTTTTTATTTCTTTATTTTT | |
| | | TTTTCTCTAAGAAGAGGCTTTGGAATGAGTTCCAATTTGT | |
| | | GATGTTAATACAGGCTTCTTGTTTTAGGAAGCATCACCTA | |
| | | TACTCTGAAGCCTTTAAACTCTGAAGAGAATTGTTTCAGA | |
| | | GTTATTCCAAGCACTTGTGCAACTTGGAAAAACAGACTTG | |
| | | GGTTGTGGGAACAGTTGACAGCGTTCTGAAAAGATGCCAT | |
| | | TTGTTTCCTTCTGATCTCTCACTGAATAATGTTTACTGTA | |
| | | CAGTCTTCCCAAGGTGATTCCTGCGACTGCAGGCACTGGT | |
| | | CATTTTCTCATGTAGCTGTCTTTTCAGTTATGGTAAACTC | |
| | | TTAAAGTTCAGAACACTCAACAGATTCCTTCAGTGATATA | |
| | | CTTGTTCGTTCATTTCTAAAATGTGAAGCTTTAGGACCAA | |
| | | ATTGTTAGAAAGCATCAGGATGACCAGTTATCTCGAGTAG | |
| | | ATTTTCTTGGATTTCAGAACATCTAGCATGACTCTGAAGG | |
| | | ATACCACATGTTTTATATATAAATAATTACTGTTTATGAT | |
| | | ATAGACATTGATATTGACTATTTAGAGAACCGTTGTTAAT | |
| | | TTTAAAACTAGCAATCTATAAAGTGCACCAGGTCAACTTG | |
| | | AATAAAAACACTATGACAGACAGGTTTGCCAGTTTGCAGA | |
| | | AACTAACTCTTTTCTCACATCAACATTTGTAAAATTGATG | |
| | | TGTTATAGTGGAAAATAACATATAGATTAAACAAAATTTT | |
| | | TATCTTTTTTCAAGAATATAGCTGGCTATCTTTAAGAAAG | |
| | | ATGATATATCCTAGTTTTGAAAGTAATTTTCTTTTTTCTT | |
| | | TCTAGCATTTGATGTCTAAATAATTTTGGACATCTTTTTC | |
| | | CTAGACCATGTTTCTGTCTTACTCTTAAACCTGGTAACAC | |
| | | TTGATTTGCCTTCTATAACCTATTTATTTCAAGTGTTCAT | |
| | | ATTTGAATTTCTTTGGGAAGAAAGTAAATCTGATGGCTCA | |
| | | CTGATTTTTGAAAAGCCTGAATAAAATTGGAAAGACTGGA | |
| | | AAGTTAGGAGAACTGACTAGCTAAACTGCTACAGTATGCA | |
| | | ATTTCTATTACAATTGGTATTACAGGGGGGAAAAGTAAAA | |
| | | TTACACTTTACCTGAAAGTGACTTCTTACAGCTAGTGCAT | |
| | | TGTGCTCTTTCCAAGTTCAGCAGCAGTTCTATCAGTGGTG | |
| | | CCACTGAAACTGGGTATATTTATGATTTCTTTCAGCGTTA | |
| | | AAAAGAAACATAGTGTTGCCCTTTTTCTTAAAGCATCAGT | |
| | | GAAATTATGGAAAATTACTTAAAACGTGAATACATCATCA | |
| | | CAGTAGAATTTATTATGAGAGCATGTAGTATGTATCTGTA | |
| | | GCCCTAACACATGGGATGAACGTTTTACTGCTACACCCAG | |
| | | ATTTGTGTTGAACGAAAACATTGTGGTTTGGAAAGGAGAA | |
| | | TTCAACAATTAATAGTTGAAATTGTGAGGTTAATGTTTAA | |
| | | AAAGCTTTACACCTGTTTACAATTTGGGGACAAAAAGGCA | |
| | | GGCTTCATTTTTCATATGTTTGATGAAAACTGGCTCAAGA | |
| | | TGTTTGTAAATAGAATCAAGAGCAAAACTGCACAAACTTG | |
| | | CACATTGGAAAGTGCAACAAGTTCCCGTGATTGCAGTAAA | |
| | | AATATTTACTATTCTAAAAAAATGAGAATTGAAGACTTAG | |
| | | CCAGTCAGATAAGTTTTTTCATGAACCCGTTGTGGAAATT | |
| | | ATTGGAATTAACTGAGCCAAAGTGATTATGCATTCTTCAT | |
| | | CTATTTTAGTTAGCACTTTGTATCGTTATATACAGTTTAC | |
| | | AATACATGTATAACTTGTAGCTATAAACATTTTGTGCCAT | |
| | | TAAAGCTCTCACAAAACTTTAAAAA | |
| BRAF | NM_004333.4 | CGCCTCCCTTCCCCCTCCCCGCCCGACAGCGGCCGCTCGG | 7 |
| | | GCCCCGGCTCTCGGTTATAAGATGGCGGCGCTGAGCGGTG | |
| | | GCGGTGGTGGCGGCGCGGAGCCGGGCCAGGCTCTGTTCAA | |
| | | CGGGGACATGGAGCCCGAGGCCGGCGCCGGCGCCGGCGCC | |
| | | GCGGCCTCTTCGGCTGCGGACCCTGCCATTCCGGAGGAGG | |
| | | TGTGGAATATCAAACAAATGATTAAGTTGACACAGGAACA | |
| | | TATAGAGGCCCTATTGGACAAATTTGGTGGGGAGCATAAT | |
| | | CCACCATCAATATATCTGGAGGCCTATGAAGAATACACCA | |
| | | GCAAGCTAGATGCACTCCAACAAAGAGAACAACAGTTATT | |
| | | GGAATCTCTGGGGAACGGAACTGATTTTTCTGTTTCTAGC | |
| | | TCTGCATCAATGGATACCGTTACATCTTCTTCCTCTTCTA | |
| | | GCCTTTCAGTGCTACCTTCATCTCTTTCAGTTTTTCAAAA | |
| | | TCCCACAGATGTGGCACGGAGCAACCCCAAGTCACCACAA | |
| | | AAACCTATCGTTAGAGTCTTCCTGCCCAACAAACAGAGGA | |
| | | CAGTGGTACCTGCAAGGTGTGGAGTTACAGTCCGAGACAG | |
| | | TCTAAAGAAAGCACTGATGATGAGAGGTCTAATCCCAGAG | |
| | | TGCTGTGCTGTTTACAGAATTCAGGATGGAGAGAAGAAAC | |
| | | CAATTGGTTGGGACACTGATATTTCCTGGCTTACTGGAGA | |
| | | AGAATTGCATGTGGAAGTGTTGGAGAATGTTCCACTTACA | |
| | | ACACACAACTTTGTACGAAAAACGTTTTTCACCTTAGCAT | |
| | | TTTGTGACTTTTGTCGAAAGCTGCTTTTCCAGGGTTTCCG | |
| | | CTGTCAAACATGTGGTTATAAATTTCACCAGCGTTGTAGT | |
| | | ACAGAAGTTCCACTGATGTGTGTTAATTATGACCAACTTG | |
| | | ATTTGCTGTTTGTCTCCAAGTTCTTTGAACACCACCCAAT | |
| | | ACCACAGGAAGAGGCGTCCTTAGCAGAGACTGCCCTAACA | |
| | | TCTGGATCATCCCCTTCCGCACCCGCCTCGGACTCTATTG | |
| | | GGCCCCAAATTCTCACCAGTCCGTCTCCTTCAAAATCCAT | |
| | | TCCAATTCCACAGCCCTTCCGACCAGCAGATGAAGATCAT | |
| | | CGAAATCAATTTGGGCAACGAGACCGATCCTCATCAGCTC | |
| | | CCAATGTGCATATAAACACAATAGAACCTGTCAATATTGA | |
| | | TGACTTGATTAGAGACCAAGGATTTCGTGGTGATGGAGGA | |
| | | TCAACCACAGGTTTGTCTGCTACCCCCCCTGCCTCATTAC | |
| | | CTGGCTCACTAACTAACGTGAAAGCCTTACAGAAATCTCC | |
| | | AGGACCTCAGCGAGAAAGGAAGTCATCTTCATCCTCAGAA | |
| | | GACAGGAATCGAATGAAAACACTTGGTAGACGGGACTCGA | |
| | | GTGATGATTGGGAGATTCCTGATGGGCAGATTACAGTGGG | |
| | | ACAAAGAATTGGATCTGGATCATTTGGAACAGTCTACAAG | |
| | | GGAAAGTGGCATGGTGATGTGGCAGTGAAAATGTTGAATG | |
| | | TGACAGCACCTACACCTCAGCAGTTACAAGCCTTCAAAAA | |
| | | TGAAGTAGGAGTACTCAGGAAAACACGACATGTGAATATC | |
| | | CTACTCTTCATGGGCTATTCCACAAAGCCACAACTGGCTA | |
| | | TTGTTACCCAGTGGTGTGAGGGCTCCAGCTTGTATCACCA | |
| | | TCTCCATATCATTGAGACCAAATTTGAGATGATCAAACTT | |
| | | ATAGATATTGCACGACAGACTGCACAGGGCATGGATTACT | |
| | | TACACGCCAAGTCAATCATCCACAGAGACCTCAAGAGTAA | |
| | | TAATATATTTCTTCATGAAGACCTCACAGTAAAAATAGGT | |
| | | GATTTTGGTCTAGCTACAGTGAAATCTCGATGGAGTGGGT | |
| | | CCCATCAGTTTGAACAGTTGTCTGGATCCATTTTGTGGAT | |
| | | GGCACCAGAAGTCATCAGAATGCAAGATAAAAATCCATAC | |
| | | AGCTTTCAGTCAGATGTATATGCATTTGGAATTGTTCTGT | |
| | | ATGAATTGATGACTGGACAGTTACCTTATTCAAACATCAA | |
| | | CAACAGGGACCAGATAATTTTTATGGTGGGACGAGGATAC | |
| | | CTGTCTCCAGATCTCAGTAAGGTACGGAGTAACTGTCCAA | |
| | | AAGCCATGAAGAGATTAATGGCAGAGTGCCTCAAAAAGAA | |
| | | AAGAGATGAGAGACCACTCTTTCCCCAAATTCTCGCCTCT | |
| | | ATTGAGCTGCTGGCCCGCTCATTGCCAAAAATTCACCGCA | |
| | | GTGCATCAGAACCCTCCTTGAATCGGGCTGGTTTCCAAAC | |
| | | AGAGGATTTTAGTCTATATGCTTGTGCTTCTCCAAAAACA | |
| | | CCCATCCAGGCAGGGGGATATGGTGCGTTTCCTGTCCACT | |
| | | GAAACAAATGAGTGAGAGAGTTCAGGAGAGTAGCAACAAA | |
| | | AGGAAAATAAATGAACATATGTTTGCTTATATGTTAAATT | |
| | | GAATAAAATACTCTCTTTTTTTTTAAGGTGAACCAAAGAA | |
| | | CACTTGTGTGGTTAAAGACTAGATATAATTTTTCCCCAAA | |
| | | CTAAAATTTATACTTAACATTGGATTTTTAACATCCAAGG | |
| | | GTTAAAATACATAGACATTGCTAAAAATTGGCAGAGCCTC | |
| | | TTCTAGAGGCTTTACTTTCTGTTCCGGGTTTGTATCATTC | |
| | | ACTTGGTTATTTTAAGTAGTAAACTTCAGTTTCTCATGCA | |
| | | ACTTTTGTTGCCAGCTATCACATGTCCACTAGGGACTCCA | |
| | | GAAGAAGACCCTACCTATGCCTGTGTTTGCAGGTGAGAAG | |
| | | TTGGCAGTCGGTTAGCCTGGGTTAGATAAGGCAAACTGAA | |
| | | CAGATCTAATTTAGGAAGTCAGTAGAATTTAATAATTCTA | |
| | | TTATTATTCTTAATAATTTTTCTATAACTATTTCTTTTTA | |
| | | TAACAATTTGGAAAATGTGGATGTCTTTTATTTCCTTGAA | |
| | | GCAATAAACTAAGTTTCTTTTTATAAAAA | |
| C21ORF7 | NM_020152.3 | CGCAGCCCCGGTTCCTGCCCGCACCTCTCCCTCCACACCT | 8 |
| | | CCCCGCAAGCTGAGGGAGCCGGCTCCGGCCTCGGCCAGCC | |
| | | CAGGAAGGCGCTCCCACAGCGCAGTGGTGGGCTGAAGGGC | |
| | | TCCTCAAGTGCCGCCAAAGTGGGAGCCCAGGCAGAGGAGG | |
| | | CGCCGAGAGCGAGGGAGGGCTGTGAGGACTGCCAGCACGC | |
| | | TGTCACCTCTCAATAGCAGCCCAAACAGATTAAGACATGG | |
| | | GAGATGTACAAGGGCAGCCGTGGGGCTGGCAACAGCTTCG | |
| | | TAATCCTGGCTTCCTGCTTTCTGGGTCAAAGCCCTGGTGG | |
| | | TGTGTTCTTGATATCGGTCCATCTAGTGGCGTTGTTTGAT | |
| | | TCCTCCCACCTTGCTGATCATTCGTAGTGTAGCCCCCAAG | |
| | | GTGTGGAATAACCCTTAAGCCCTTACCGGGGTCCTTCTGG | |
| | | ACTGAGAATTGTTGTAAAGTAATACTGCTCAGGTGAAAGA | |
| | | CAACTTGAGTGGTTAAATTACTGTCATGCAAAGCGACTAG | |
| | | ATGGTTCAGCTGATTGCACCTTTAGAAGTTATGTGGAACG | |
| | | AGGCAGCAGATCTTAAGCCCCTTGCTCTGTCACGCAGGCT | |
| | | GGAATGCAGTGGTGGAATCATGGCTCACTACAGCCCTGAC | |
| | | CTCCTGGGCCCAGAGATGGAGTCTCGCTATTTTGCCCAGG | |
| | | TTGGTCTTGAACACCTGGCTTCAAGCAGTCCTCCTGCTTT | |
| | | TGGCTTCTTGAAGTGCTTGGATTACAGTATTTCAGTTTTA | |
| | | TGCTCTGCAACAAGTTTGGCCATGTTGGAGGACAATCCAA | |
| | | AGGTCAGCAAGTTGGCTACTGGCGATTGGATGCTCACTCT | |
| | | GAAGCCAAAGTCTATTACTGTGCCCGTGGAAATCCCCAGC | |
| | | TCCCCTCTGGATGATACACCCCCTGAAGACTCCATTCCTT | |
| | | TGGTCTTTCCAGAATTAGACCAGCAGCTACAGCCCCTGCC | |
| | | GCCTTGTCATGACTCCGAGGAATCCATGGAGGTGTTCAAA | |
| | | CAGCACTGCCAAATAGCAGAAGAATACCATGAGGTCAAAA | |
| | | AGGAAATCACCCTGCTTGAGCAAAGGAAGAAGGAGCTCAT | |
| | | TGCCAAGTTAGATCAGGCAGAAAAGGAGAAGGTGGATGCT | |
| | | GCTGAGCTGGTTCGGGAATTCGAGGCTCTGACGGAGGAGA | |
| | | ATCGGACGTTGAGGTTGGCCCAGTCTCAATGTGTGGAACA | |
| | | ACTGGAGAAACTTCGAATACAGTATCAGAAGAGGCAGGGC | |
| | | TCGTCCTAACTTTAAATTTTTCAGTGTGAGCATACGAGGC | |
| | | TGATGACTGCCCTGTGCTGGCCAAAAGATTTTTATTTTAA | |
| | | ATGAATAGTGAGTCAGATCTATTGCTTCTCTGTATTACCC | |
| | | ACATGACAACTGTCTATAATGAGTTTACTGCTTGCCAGCT | |
| | | TCTAGCTTGAGAGAAGGGATATTTTAAATGAGATCATTAA | |
| | | CGTGAAACTATTACTAGTATATGTTTTTGGAGATCAGAAT | |
| | | TCTTTTCCAAAGATATATGTTTTTTTCTTTTTTAGGAAGA | |
| | | TATGATCATGCTGTACAACAGGGTAGAAAATGATAAAAAT | |
| | | AGACTATTGACTGACCCAGCTAAGAATCGTGGGCTGAGCA | |
| | | GAGTTAAACCATGGGACAAACCCATAACATGTTCACCATA | |
| | | GTTTCACGTATGTGTATTTTTAAATTTCATGCCTTTAATA | |
| | | TTTCAAATATGCTCAAATTTAAACTGTCAGAAACTTCTGT | |
| | | GCATGTATTTATATTTGCCAGAGTATAAACTTTTATACTC | |
| | | TGATTTTTATCCTTCAATGATTGATTATACTAAGAATAAA | |
| | | TGGTCACATATCCTAAAAGCTTCTTCATGAAATTATTAGC | |
| | | AGAAACCATGTTTGTAACCAAAGCACATTTGCCAATGCTA | |
| | | ACTGGCTGTTGTAATAATAAACAGATAAGGCTGCATTTGC | |
| | | TTCATGCCATGTGACCTCACAGTAAACATCTCTGCCTTTG | |
| | | CCTGTGTGTGTTCTGGGGGAGGGGGGACATGGAAAAATAT | |
| | | TGTTTGGACATTACTTGGGTGAGTGCCCATGAAAACATCA | |
| | | GTGAACTTGTAACTATTGTTTTGTTTTGGATTTAAGGAGA | |
| | | TGTTTTAGATCAGTAACAGCTAATAGGAATATGCGAGTAA | |
| | | ATTCAGAATTGAAACAATTTCTCCTTGTTCTACCTATCAC | |
| | | CACATTTTCTCAAATTGAACTCTTTGTTATATGTCCATTT | |
| | | CTATTCATGTAACTTCTTTTTCATTAAACATGGATCAAAA | |
| | | CTGACAAAAAAAAAAAAAAA | |
| CD59 | NM_203331.2 | GGGGCCGGGGGGCGGAGCCTTGCGGGCTGGAGCGAAAGAA | 9 |
| | | TGCGGGGGCTGAGCGCAGAAGCGGCTCGAGGCTGGAAGAG | |
| | | GATCTTGGGCGCCGCCAGTCTTTAGCACCAGTTGGTGTAG | |
| | | GAGTTGAGACCTACTTCACAGTAGTTCTGTGGACAATCAC | |
| | | AATGGGAATCCAAGGAGGGTCTGTCCTGTTCGGGCTGCTG | |
| | | CTCGTCCTGGCTGTCTTCTGCCATTCAGGTCATAGCCTGC | |
| | | AGTGCTACAACTGTCCTAACCCAACTGCTGACTGCAAAAC | |
| | | AGCCGTCAATTGTTCATCTGATTTTGATGCGTGTCTCATT | |
| | | ACCAAAGCTGGGTTACAAGTGTATAACAAGTGTTGGAAGT | |
| | | TTGAGCATTGCAATTTCAACGACGTCACAACCCGCTTGAG | |
| | | GGAAAATGAGCTAACGTACTACTGCTGCAAGAAGGACCTG | |
| | | TGTAACTTTAACGAACAGCTTGAAAATGGTGGGACATCCT | |
| | | TATCAGAGAAAACAGTTCTTCTGCTGGTGACTCCATTTCT | |
| | | GGCAGCAGCCTGGAGCCTTCATCCCTAAGTCAACACCAGG | |
| | | AGAGCTTCTCCCAAACTCCCCGTTCCTGCGTAGTCCGCTT | |
| | | TCTCTTGCTGCCACATTCTAAAGGCTTGATATTTTCCAAA | |
| | | TGGATCCTGTTGGGAAAGAATAAAATTAGCTTGAGCAACC | |
| | | TGGCTAAGATAGAGGGGCTCTGGGAGACTTTGAAGACCAG | |
| | | TCCTGTTTGCAGGGAAGCCCCACTTGAAGGAAGAAGTCTA | |
| | | AGAGTGAAGTAGGTGTGACTTGAACTAGATTGCATGCTTC | |
| | | CTCCTTTGCTCTTGGGAAGACCAGCTTTGCAGTGACAGCT | |
| | | TGAGTGGGTTCTCTGCAGCCCTCAGATTATTTTTCCTCTG | |
| | | GCTCCTTGGATGTAGTCAGTTAGCATCATTAGTACATCTT | |
| | | TGGAGGGTGGGGCAGGAGTATATGAGCATCCTCTCTCACA | |
| | | TGGAACGCTTTCATAAACTTCAGGGATCCCGTGTTGCCAT | |
| | | GGAGGCATGCCAAATGTTCCATATGTGGGTGTCAGTCAGG | |
| | | GACAACAAGATCCTTAATGCAGAGCTAGAGGACTTCTGGC | |
| | | AGGGAAGTGGGGAAGTGTTCCAGATAGCAGGGCATGAAAA | |
| | | CTTAGAGAGGTACAAGTGGCTGAAAATCGAGTTTTTCCTC | |
| | | TGTCTTTAAATTTTATATGGGCTTTGTTATCTTCCACTGG | |
| | | AAAAGTGTAATAGCATACATCAATGGTGTGTTAAAGCTAT | |
| | | TTCCTTGCCTTTTTTTTATTGGAATGGTAGGATATCTTGG | |
| | | CTTTGCCACACACAGTTACAGAGTGAACACTCTACTACAT | |
| | | GTGACTGGCAGTATTAAGTGTGCTTATTTTAAATGTTACT | |
| | | GGTAGAAAGGCAGTTCAGGTATGTGTGTATATAGTATGAA | |
| | | TGCAGTGGGGACACCCTTTGTGGTTACAGTTTGAGACTTC | |
| | | CAAAGGTCATCCTTAATAACAACAGATCTGCAGGGGTATG | |
| | | TTTTACCATCTGCATCCAGCCTCCTGCTAACTCCTAGCTG | |
| | | ACTCAGCATAGATTGTATAAAATACCTTTGTAACGGCTCT | |
| | | TAGCACACTCACAGATGTTTGAGGCTTTCAGAAGCTCTTC | |
| | | TAAAAAATGATACACACCTTTCACAAGGGCAAACTTTTTC | |
| | | CTTTTCCCTGTGTATTCTAGTGAATGAATCTCAAGATTCA | |
| | | GTAGACCTAATGACATTTGTATTTTATGATCTTGGCTGTA | |
| | | TTTAATGGCATAGGCTGACTTTTGCAGATGGAGGAATTTC | |
| | | TTGATTAATGTTGAAAAAAAACCCTTGATTATACTCTGTT | |
| | | GGACAAACCGAGTGCAATGAATGATGCTTTTCTGAAAATG | |
| | | AAATATAACAAGTGGGTGAATGTGGTTATGGCCGAAAAGG | |
| | | ATATGCAGTATGCTTAATGGTAGCAACTGAAAGAAGACAT | |
| | | CCTGAGCAGTGCCAGCTTTCTTCTGTTGATGCCGTTCCCT | |
| | | GAACATAGGAAAATAGAAACTTGCTTATCAAAACTTAGCA | |
| | | TTACCTTGGTGCTCTGTGTTCTCTGTTAGCTCAGTGTCTT | |
| | | TCCTTACATCAATAGGTTTTTTTTTTTTTTTTTGGCCTGA | |
| | | GGAAGTACTGACCATGCCCACAGCCACCGGCTGAGCAAAG | |
| | | AAGCTCATTTCATGTGAGTTCTAAGGAATGAGAAACAATT | |
| | | TTGATGAATTTAAGCAGAAAATGAATTTCTGGGAACTTTT | |
| | | TTGGGGGCGGGGGGGTGGGGAATTCAGCCACACTCCAGAA | |
| | | AGCCAGGAGTCGACAGTTTTGGAAGCCTCTCTCAGGATTG | |
| | | AGATTCTAGGATGAGATTGGCTTACTGCTATCTTGTGTCA | |
| | | TGTACCCACTTTTTGGCCAGACTACACTGGGAAGAAGGTA | |
| | | GTCCTCTAAAGCAAAATCTGAGTGCCACTAAATGGGGAGA | |
| | | TGGGGCTGTTAAGCTGTCCAAATCAACAAGGGTCATATAA | |
| | | ATGGCCTTAAACTTTGGGGTTGCTTTCTGCAAAAAGTTGC | |
| | | TGTGACTCATGCCATAGACAAGGTTGAGTGCCTGGACCCA | |
| | | AAGGCAATACTGTAATGTAAAGACATTTATAGTACTAGGC | |
| | | AAACAGCACCCCAGGTACTCCAGGCCCTCCTGGCTGGAGA | |
| | | GGGCTGTGGCAATAGAAAATTAGTGCCAACTGCAGTGAGT | |
| | | CAGCCTAGGTTAAATAGAGAGTGTAAGAGTGCTGGACAGG | |
| | | AACCTCCACCCTCATGTCACATTTCTTCAATGTGACCCTT | |
| | | CTGGCCCCTCTCCTCCTGACAGCGGAACAATGACTGCCCC | |
| | | GATAGGTGAGGCTGGAGGAAGAATCAGTCCTGTCCTTGGC | |
| | | AAGCTCTTCACTATGACAGTAAAGGCTCTCTGCCTGCTGC | |
| | | CAAGGCCTGTGACTTTCTAACCTGGCCTCACGCTGGGTAA | |
| | | GCTTAAGGTAGAGGTGCAGGATTAGCAAGCCCACCTGGCT | |
| | | ACCAGGCCGACAGCTACATCCTCCAACTGACCCTGATCAA | |
| | | CGAAGAGGGATTCATGTGTCTGTCTCAGTTGGTTCCAAAT | |
| | | GAAACCAGGGAGCAGGGGAGTTAGGAATCGAACACCAGTC | |
| | | ATGCCTACTGGCTCTCTGCTCGAGAGCCAATACCCTGTGC | |
| | | CCTCCACTCATCTGGATTTACAGGAACTGTCATAGTGTTC | |
| | | AGTATTGGGTGGTGATAAGCCCATTGGATTGTCCCCTTGG | |
| | | GGGGATGAGCTAGGGGTGCAAGGAACACCTGATGAGTAGA | |
| | | TAAGTGGAGCTCATGGTATTTCCTGAAAGATGCTAATCTA | |
| | | TTTGCCAAACTTGGTCTTGAATGTACTGGGGGCTTCAAGG | |
| | | TATGGGTATATTTTTCTTGTGTCCTTGCAGTTAGCCCCCA | |
| | | TGTCTTATGTGTGTCCTGAAAAAATAAGAGCCTGCCCAAG | |
| | | ACTTTGGGCCTCTTGACAGAATTAACCACTTTTATACATC | |
| | | TGAGTTCTCTTGGTAAGTTCTTTAGCAGTGTTCAAAGTCT | |
| | | ACTAGCTCGCATTAGTTTCTGTTGCTGCCAACAGATCTGA | |
| | | ACTAATGCTAACAGATCCCCCTGAGGGATTCTTGATGGGC | |
| | | TGAGCAGCTGGCTGGAGCTAGTACTGACTGACATTCATTG | |
| | | TGATGAGGGCAGCTTTCTGGTACAGGATTCTAAGCTCTAT | |
| | | GTTTTATATACATTTTCATCTGTACTTGCACCTCACTTTA | |
| | | CACAAGAGGAAACTATGCAAAGTTAGCTGGATCGCTCAAG | |
| | | GTCACTTAGGTAAGTTGGCAAGTCCATGCTTCCCACTCAG | |
| | | CTCCTCAGGTCAGCAAGTCTACTTCTCTGCCTATTTTGTA | |
| | | TACTCTCTTTAATATGTGCCTAGCTTTGGAAAGTCTAGAA | |
| | | TGGGTCCCTGGTGCCTTTTTACTTTGAAGAAATCAGTTTC | |
| | | TGCCTCTTTTTGGAAAAGAAAACAAAGTGCAATTGTTTTT | |
| | | TACTGGAAAGTTACCCAATAGCATGAGGTGAACAGGACGT | |
| | | AGTTAGGCCTTCCTGTAAACAGAAAATCATATCAAAACAC | |
| | | TATCTTCCCATCTGTTTCTCAATGCCTGCTACTTCTTGTA | |
| | | GATATTTCATTTCAGGAGAGCAGCAGTTAAACCCGTGGAT | |
| | | TTTGTAGTTAGGAACCTGGGTTCAAACCCTCTTCCACTAA | |
| | | TTGGCTATGTCTCTGGACAAGTTTTTTTTTTTTTTTTTTT | |
| | | TTAAACCCTTTCTGAACTTTCACTTTCTATGTCTACCTCA | |
| | | AAGAATTGTTGTGAGGCTTGAGATAATGCATTTGTAAAGG | |
| | | GTCTGCCAGATAGGAAGATGCTAGTTATGGATTTACAAGG | |
| | | TTGTTAAGGCTGTAAGAGTCTAAAACCTACAGTGAATCAC | |
| | | AATGCATTTACCCCCACTGACTTGGACATAAGTGAAAACT | |
| | | AGCCAGAAGTCTCTTTTTCAAATTACTTACAGGTTATTCA | |
| | | ATATAAAATTTTTGTAATGGATAATCTTATTTATCTAAAC | |
| | | TAAAGCTTCCTGTTTATACACACTCCTGTTATTCTGGGAT | |
| | | AAGATAAATGACCACAGTACCTTAATTTCTAGGTGGGTGC | |
| | | CTGTGATGGTTCATTGTAGGTAAGGACATTTTCTCTTTTT | |
| | | CAGCAGCTGTGTAGGTCCAGAGCCTCTGGGAGAGGAGGGG | |
| | | GGTAGCATGCACCCAGCAGGGGACTGAACTGGGAAACTCA | |
| | | AGGTTCTTTTTACTGTGGGGTAGTGAGCTGCCTTTCTGTG | |
| | | ATCGGTTTCCCTAGGGATGTTGCTGTTCCCCTCCTTGCTA | |
| | | TTCGCAGCTACATACAACGTGGCCAACCCCAGTAGGCTGA | |
| | | TCCTATATATGATCAGTGCTGGTGCTGACTCTCAATAGCC | |
| | | CCACCCAAGCTGGCTATAGGTTTACAGATACATTAATTAG | |
| | | GCAACCTAAAATATTGATGCTGGTGTTGGTGTGACATAAT | |
| | | GCTATGGCCAGAACTGAAACTTAGAGTTATAATTCATGTA | |
| | | TTAGGGTTCTCCAGAGGGACAGAATTAGTAGGATATATGT | |
| | | ATATATGAAAGGGAGGTTATTAGGGAGAACTGGCTCCCAC | |
| | | AGTTAGAAGGCGAAGTCGCACAATAGGCCGTCTGCAAGCT | |
| | | GGGTTAGAGAGAAGCCAGTAGTGGCTCAGCCTGAGTTCAA | |
| | | AAACCTCAAAACTGGGGAAGCTGACAGTGCAGCCAGCCTT | |
| | | CAGTCTGTGGCCAAAGGCCCAAGAGCCCCTGGCAACCAAC | |
| | | CCACTGGTGCAAGTCCTAGATTCCAAAGGCTGAAGAACCT | |
| | | GGAGTCTGATGTCCAAGAGCAGGAAGAGTGGAAGAAAGCC | |
| | | AGAAGACTCAGCAAACAAGGTAGACAGTGTCTACCACCAT | |
| | | AGTGGCCATACCAAAGAGGCTACCGATTCCTTCCTGCTAC | |
| | | CTGGATCCCTGAAGTTGCCCTGGTCTCTGCACCTTCTAAA | |
| | | CCTAGTTCTTAAGAGCTTTCCATTACATGAGCTGTCTCAA | |
| | | AGCCCTCCAATAAATTCTCAGTGTAAGCTTCTGTTGCTTG | |
| | | TGGACAGAAAATTCTGACAGACCTACCCTATAAGTGTTAC | |
| | | TGTCAGGATAACATGAGAACGCACAACAGTAAGTGGTCAC | |
| | | TAAGTGTTAGCTACGGTTATTTTGCCCAAGGTAGCATGGC | |
| | | TAGTTGATGCCGGTTGATGGGGCTTAAACCCAGCTCCCTC | |
| | | ATCTTCCAGGCCTCTGTACTCCCTATTCCACTAAACTACC | |
| | | TCTCAGGTTTATTTTTTTAAATTCTTACTCTGCAAGTACA | |
| | | TAGGACCACATTTACCTGGGAAAACAAGAATAAAGGCTGC | |
| | | TCTGCATTTTTTAGAAACTTTTTTGAAAGGGAGATGGGAA | |
| | | TGCCTGCACCCCCAAGTCCAGACCAACACAATGGTTAATT | |
| | | GAGATGAATAATAAAGGAAAGACTGTTCTGGGCTTCCCAG | |
| | | AATAGCTTGGTCCTTAAATTGTGGCACAAACAACCTCCTG | |
| | | TCAGAGCCAGCCTCCTGCCAGGAAGAGGGGTAGGAGACTA | |
| | | GAGGCCGTGTGTGCAGCCTTGCCCTGAAGGCTAGGGTGAC | |
| | | AATTTGGAGGCTGTCCAAACACCCTGGCCTCTAGAGCTGG | |
| | | CCTGTCTATTTGAAATGCCGGCTCTGATGCTAATCGGCGA | |
| | | CCCTCAGGCAAGTTACTTAACCTTACATGCCTCAGTTTTC | |
| | | TCATCTGGAAAATGAGAACCCTAGGTTTAGGGTTGTTAGA | |
| | | AAAGTTAAATGAGTTAAGACAAGTGCCTGGGACACAGTAG | |
| | | CCTCTTGTGTGTGTTTATCATTATGTCCTCAGCAGGTCGT | |
| | | AGAAGCAGCTTCTCAGGTGTGAGGCTGGCGCGATTATCTG | |
| | | GAGTGGGTTGGGTTTTCTAGGATGGACCCCCTGCTGCATT | |
| | | TTCCTCATTCATCCACCAGGGCTTAATGGGGAATCAAGGA | |
| | | ATCCATGTGTAACTGTATAATAACTGTAGCCACACTCCAA | |
| | | TGACCACCTACTAGTTGTCCCTGGCACTGCTTATACATAT | |
| | | GTCCATCAAATCAATCCTATGAAGTAGATACTGTCTTCAT | |
| | | TTTATAGATCAGAGACAATTGGGGTTCAGAGAGCTGATGT | |
| | | GATTTTCCCAGGGTCACAGAGAGTCCCAGATTCAGGCACA | |
| | | ACTCTTGTATTCCAAGACACAACCACTACATGTCCAAAGG | |
| | | CTGCCCAGAGCCACCGGGCACGGCAAATTGTGACATATCC | |
| | | CTAAAGAGGCTGAGCACCTGGTCAGGATCTGATGGCTGAC | |
| | | AGTGTGTCCAGATGCAGAGCTGGAGTGGGGGAGGGGAAGG | |
| | | GGGGCTCCTTGGGACAGAGAAGGCTTTCTGTGCTTTCTCT | |
| | | GAAGGGAGCAGTCTGAGGACCAAGGGAACCCGGCAAACAG | |
| | | CACCTCAGGTACTCCAGGCCCTCCTGGCTGGAGAGGGCTG | |
| | | TGGCAATGGAAAATTAGTGCCAACTGCAATGAGTCAGCCT | |
| | | CGGTTAAATAGAGAGTGAAGAATGCTGGACAGGAACCTCC | |
| | | ACCCTCATGTCACATTTCTTCAGTGTGACCCTTCTGGCCC | |
| | | CTCTCCTCCTGACAGCGGAACAATGACTGCCCCGATAGGT | |
| | | GAGGCTGGAGGAAGAATCAGTCCTGTCCTTGGCAAGCTCT | |
| | | TCACTATGACAGTAAAGGCTCTCTGCCTGCTGCCAAGGCC | |
| | | TGTGACTTTCTAACCTGGCCTCACGCTGGGTAAGCTTAAG | |
| | | GTAGAGGTGCAGGATTAGCAAGCCCACCTGGCTACCAGGC | |
| | | CGACAGCTACATCTTTCAACTGACCCTGATCAACGAAGAG | |
| | | GGACTTGTGTCTCTCAGTTGGTTCCAAATGAAACCAGGGA | |
| | | GCAGGGGCGTTAGGAAGCTCCAACAGGATGGTACTTAATG | |
| | | GGGCATTTGAGTGGAGAGGTAGGTGACATAGTGCTTTGGA | |
| | | GCCCAGGGAGGGAAAGGTTCTGCTGAAGTTGAATTCAAGA | |
| | | CTGTTCTTTCATCACAAACTTGAGTTTCCTGGACATTTGT | |
| | | TTGCAGAAACAACCGTAGGGTTTTGCCTTAACCTCGTGGG | |
| | | TTTATTATTACCTCATAGGGACTTTGCCTCCTGACAGCAG | |
| | | TTTATGGGTGTTCATTGTGGCACTTGAGTTTTCTTGCATA | |
| | | CTTGTTAGAGAAACCAAGTTTGTCATCAACTTCTTATTTA | |
| | | ACCCCCTGGCTATAACTTCATGGATTATGTTATAATTAAG | |
| | | CCATCCAGAGTAAAATCTGTTTAGATTATCTTGGAGTAAG | |
| | | GGGGAAAAAATCTGTAATTTTTTCTCCTCAACTAGATATA | |
| | | TACATAAAAAATGATTGTATTGCTTCATTTAAAAAATATA | |
| | | ACGCAAAATCTCTTTTCCTTCTAAAAAAAAAAAAAAAAAA | |
| COMMD9 | NM_001101653.1 | GCTTCCCTGGGTGCCACGGTCATGTGACTTCGGCAAGATG | 10 |
| | | GCTGCCCTGACAGCGGAGCATTTTGCAGCACTCCAGAGCC | |
| | | TGCTCAAGCTGCTCCAGGCTCTGCACCGCCTCACTAGGCT | |
| | | GGTGGCATTCCGTGACCTGTCCTCTGCCGAGGCAATTCTG | |
| | | GCTCTCTTTCCAGAAAATTTCCACCAAAACCTCAAAAACC | |
| | | TGCTGACAAAGATCATCCTAGAACATGTGTCTACTTGGAG | |
| | | AACCGAAGCCCAGGCAAATCAGATCTCTCTGCCACGCCTG | |
| | | GTCGATCTGGACTGGAGAGTGGATATCAAAACCTCCTCAG | |
| | | ACAGCATCAGCCGCATGGCCGTCCCCACCTGCCTGCTCCA | |
| | | GATGAAGATCCAAGAAGATCCCAGCCTATGCGGAGACAAA | |
| | | CCCTCCATCTCAGCTGTCACCGTGGAGCTGAGCAAAGAAA | |
| | | CACTGGACACCATGTTAGATGGCCTGGGCCGCATCCGAGA | |
| | | CCAACTCTCTGCCGTGGCCAGTAAATGATCCAGCCAGCTG | |
| | | CCAGGGCCACTGCCATGACCCAGCTGCTCATGAGTGATAA | |
| | | ATGTCTCCCCATATGCAGGCTGCCCTTGCAGCTGCAGCTG | |
| | | ACAACAGGCAGGATGGTGGGGACAGCAGGGGGCTACTGCC | |
| | | ATCCAGAAGTTACAGTTGGATTGGGAAGAAGCAGCCAGAT | |
| | | CCCCCGCTGTTCTCACTCATCTTCTTTCTCTTTCTGAAGC | |
| | | TGGAGAGCAGAAGCCCCCATCTTTGAAAAGCTCCTGAGTG | |
| | | CAACTTAATTACCACCATGGCAGGGTGAGGGAACATTTGC | |
| | | ATCGTCAGCTGCCTCTGCATAGCTGTTTGAGAAATTCAGG | |
| | | CCCAAATCATGCAGCCTATCCAATAAGTAAGTTTATTTCC | |
| | | AACATTAGCTCTAATTAGTTCATTTCCAATCCCAGAACAC | |
| | | ATGGAGGGAATCGGACAGGTGATGCCAGCAGTTCCTGCTC | |
| | | CTCTGTCAGGGAAGCCAGGCAGAGCCCACAGAGCATGGTC | |
| | | CATCCAGAGTGTTCCCTGAGCCCCCTCCACCATACTGGAA | |
| | | CCCCTCTTCAGTGTAGGAAGTCTGAAATGGGTGCTAATTC | |
| | | CCTTCTTCATGAAACCAGGGCCCTCTTCCTTCATCTAATG | |
| | | CAGCCACTCCTAGGTGAAGAAGTGGGAATAATTGGAAATA | |
| | | AACAACAGTTCTAAAACTTCCATGATTTTTGTAGCTTCTT | |
| | | TTGTCCCCAAGTTGAAGCTTTTGGCCAGTACCTTCTCTAG | |
| | | TTTTTAAAGATGATCCCAACTTCCTAATTCCCAGCTAAGC | |
| | | CCTTGACCCATGGTGTGACATGAAATCAGGCAATTGAATC | |
| | | GCACCACTTTCTGTGTTTTCACCTGTTACGTAGAACAAAA | |
| | | GGAAGCAAGGTGGCCAGGCGCAATGGCTCACGCCTGTAAT | |
| | | CCCAGCACTTTGGGAGGCCGAGGCAGGCAGATCATGAGGT | |
| | | CAGGAGATCGAGACCATGGTGAAACCCCATCTCTACTAAA | |
| | | AATACAAAAAATTAGCTGGGCGCGGTGGCGGGCATCTGTA | |
| | | GTCCCAGCTCCTCGGGAGGCTGAGGCAGGAGAATGGCGTG | |
| | | AACCTGGGAGGCAGAGCTTGCAGTGAGCCGAGATCGTGCC | |
| | | ACTGCACTCCAGTCTGGGTGACAGAGAAGGACTCGTCTCA | |
| | | AAAAATAAAAATAAATAAAAAGGAAGCAAGGCTAATCATC | |
| | | AGTATGTGCTTGTTACAAGAGCTATGATGAAGGCACTCCT | |
| | | TCGAGTTTAACCAAATGAGATCATCTCTGTCATGTGCCTC | |
| | | ACGCCTCACAGGGACTCCATGTGTGAAGATTCCCCCTTCA | |
| | | CTCACCAGATCATCTCCATGGCAACAGCTTGCAGCCTGCT | |
| | | CTTGGAGTGCTTTGTTTTGGCAGCTTCTCTGCTAGTTTGT | |
| | | GTATGGAGTGAATGGAGGAGGTAAATCCACAGATTAAGAA | |
| | | TATGCTGTCAGGAGTCAGGCAGCCAAGGTCAGAAGCCAGC | |
| | | TCTGCTTCTCAGTGGTAAGGTGCTTGACTTCTACATCTCA | |
| | | ATTTTCACCCACTTTGTACTTTTTTCCTAAATTAAATGAG | |
| | | TATAATAGTAGTACCTACTTGATAGGACTTTTGTGAAAAT | |
| | | TAAATGATATAATGCACCTAAAAACAGTACTGTTACAACT | |
| | | AATAGGAAAGGCTTTGATTATTAATGGATGAGAGTAGAAA | |
| | | GCTTGGTGCATTTATTGTCTCATCTACTATAACAGAGTTG | |
| | | GTGTGAGAATTAGTATTATCATCCTCCCTTTATTGACCAG | |
| | | GAAACCAGCTCATTGAGATTGAGTCATCTGCTGGTAAATG | |
| | | GTCTCATTAAGAGGTGGACCCATATTTCTCTAGCTTTCTC | |
| | | TTTACAACACAGGACTTTGCAAGGAACATATAATTCTGTG | |
| | | ACTAGCGCCATTTGGAAAATGTTGAAACTGAAGTAGAGAT | |
| | | GAGAGATCTTACGTCTGCCTACCCAGTGAGATACGAGGAA | |
| | | GGTCAAGGGAAAAAAAATTCCAAGCTCTTCTTTATCTGCT | |
| | | ATAGGAAATGAACATTCAATTTTTTGCATGCAACGACAAG | |
| | | AGGTCAAGGACCCCAGAAGCCAGCCCGCTACTTCCAAGTT | |
| | | GAGAGCCCCTGGTCATACCCTCCAGTTGAGCTCAGATTTG | |
| | | TCACAAATTTACCCCTCTCCTTTCCTTCCATTCCCCATGA | |
| | | CCTGCAGAGAGAGATGTCAGATACCTTCCTCTTGGCCTCC | |
| | | CATGGGCATCCATAAGAAACTTACTTGAAGCAAGAAGCCC | |
| | | AGTATAGGTGTCTGGGCAGTTGGACATTTCCTCTAGCCAG | |
| | | ATCTGTCCGAATAGAGCCATCTGGGTACATGACGCAGAGG | |
| | | GCATTTGATAAATAACTGGAAAAGTCAATAAATCTTTGCT | |
| | | ACCCTTCAAAAAAAAAAAAAAAAAAAAAAAAAAAAAAAAA | |
| CTGF | NM_001901.2 | AAACTCACACAACAACTCTTCCCCGCTGAGAGGAGACAGC | 11 |
| | | CAGTGCGACTCCACCCTCCAGCTCGACGGCAGCCGCCCCG | |
| | | GCCGACAGCCCCGAGACGACAGCCCGGCGCGTCCCGGTCC | |
| | | CCACCTCCGACCACCGCCAGCGCTCCAGGCCCCGCCGCTC | |
| | | CCCGCTCGCCGCCACCGCGCCCTCCGCTCCGCCCGCAGTG | |
| | | CCAACCATGACCGCCGCCAGTATGGGCCCCGTCCGCGTCG | |
| | | CCTTCGTGGTCCTCCTCGCCCTCTGCAGCCGGCCGGCCGT | |
| | | CGGCCAGAACTGCAGCGGGCCGTGCCGGTGCCCGGACGAG | |
| | | CCGGCGCCGCGCTGCCCGGCGGGCGTGAGCCTCGTGCTGG | |
| | | ACGGCTGCGGCTGCTGCCGCGTCTGCGCCAAGCAGCTGGG | |
| | | CGAGCTGTGCACCGAGCGCGACCCCTGCGACCCGCACAAG | |
| | | GGCCTCTTCTGTGACTTCGGCTCCCCGGCCAACCGCAAGA | |
| | | TCGGCGTGTGCACCGCCAAAGATGGTGCTCCCTGCATCTT | |
| | | CGGTGGTACGGTGTACCGCAGCGGAGAGTCCTTCCAGAGC | |
| | | AGCTGCAAGTACCAGTGCACGTGCCTGGACGGGGCGGTGG | |
| | | GCTGCATGCCCCTGTGCAGCATGGACGTTCGTCTGCCCAG | |
| | | CCCTGACTGCCCCTTCCCGAGGAGGGTCAAGCTGCCCGGG | |
| | | AAATGCTGCGAGGAGTGGGTGTGTGACGAGCCCAAGGACC | |
| | | AAACCGTGGTTGGGCCTGCCCTCGCGGCTTACCGACTGGA | |
| | | AGACACGTTTGGCCCAGACCCAACTATGATTAGAGCCAAC | |
| | | TGCCTGGTCCAGACCACAGAGTGGAGCGCCTGTTCCAAGA | |
| | | CCTGTGGGATGGGCATCTCCACCCGGGTTACCAATGACAA | |
| | | CGCCTCCTGCAGGCTAGAGAAGCAGAGCCGCCTGTGCATG | |
| | | GTCAGGCCTTGCGAAGCTGACCTGGAAGAGAACATTAAGA | |
| | | AGGGCAAAAAGTGCATCCGTACTCCCAAAATCTCCAAGCC | |
| | | TATCAAGTTTGAGCTTTCTGGCTGCACCAGCATGAAGACA | |
| | | TACCGAGCTAAATTCTGTGGAGTATGTACCGACGGCCGAT | |
| | | GCTGCACCCCCCACAGAACCACCACCCTGCCGGTGGAGTT | |
| | | CAAGTGCCCTGACGGCGAGGTCATGAAGAAGAACATGATG | |
| | | TTCATCAAGACCTGTGCCTGCCATTACAACTGTCCCGGAG | |
| | | ACAATGACATCTTTGAATCGCTGTACTACAGGAAGATGTA | |
| | | CGGAGACATGGCATGAAGCCAGAGAGTGAGAGACATTAAC | |
| | | TCATTAGACTGGAACTTGAACTGATTCACATCTCATTTTT | |
| | | CCGTAAAAATGATTTCAGTAGCACAAGTTATTTAAATCTG | |
| | | TTTTTCTAACTGGGGGAAAAGATTCCCACCCAATTCAAAA | |
| | | CATTGTGCCATGTCAAACAAATAGTCTATCAACCCCAGAC | |
| | | ACTGGTTTGAAGAATGTTAAGACTTGACAGTGGAACTACA | |
| | | TTAGTACACAGCACCAGAATGTATATTAAGGTGTGGCTTT | |
| | | AGGAGCAGTGGGAGGGTACCAGCAGAAAGGTTAGTATCAT | |
| | | CAGATAGCATCTTATACGAGTAATATGCCTGCTATTTGAA | |
| | | GTGTAATTGAGAAGGAAAATTTTAGCGTGCTCACTGACCT | |
| | | GCCTGTAGCCCCAGTGACAGCTAGGATGTGCATTCTCCAG | |
| | | CCATCAAGAGACTGAGTCAAGTTGTTCCTTAAGTCAGAAC | |
| | | AGCAGACTCAGCTCTGACATTCTGATTCGAATGACACTGT | |
| | | TCAGGAATCGGAATCCTGTCGATTAGACTGGACAGCTTGT | |
| | | GGCAAGTGAATTTGCCTGTAACAAGCCAGATTTTTTAAAA | |
| | | TTTATATTGTAAATATTGTGTGTGTGTGTGTGTGTGTATA | |
| | | TATATATATATGTACAGTTATCTAAGTTAATTTAAAGTTG | |
| | | TTTGTGCCTTTTTATTTTTGTTTTTAATGCTTTGATATTT | |
| | | CAATGTTAGCCTCAATTTCTGAACACCATAGGTAGAATGT | |
| | | AAAGCTTGTCTGATCGTTCAAAGCATGAAATGGATACTTA | |
| | | TATGGAAATTCTGCTCAGATAGAATGACAGTCCGTCAAAA | |
| | | CAGATTGTTTGCAAAGGGGAGGCATCAGTGTCCTTGGCAG | |
| | | GCTGATTTCTAGGTAGGAAATGTGGTAGCCTCACTTTTAA | |
| | | TGAACAAATGGCCTTTATTAAAAACTGAGTGACTCTATAT | |
| | | AGCTGATCAGTTTTTTCACCTGGAAGCATTTGTTTCTACT | |
| | | TTGATATGACTGTTTTTCGGACAGTTTATTTGTTGAGAGT | |
| | | GTGACCAAAAGTTACATGTTTGCACCTTTCTAGTTGAAAA | |
| | | TAAAGTGTATATTTTTTCTATAAAAAAAAAAAAAAAAA | |
| ENPP4 | NM_014936.4 | AGACGCTCGCCTGGCAGCTGCGCACACTCGGAGCGCCCCG | 12 |
| | | AGCGGCGCAGATAGGGACGTTGGGGCTGTGCCCCGCGGCG | |
| | | CGGCGCCTGCCACTGCGCAGGCGCCTCAGGAAGAGCTCGG | |
| | | CATCGCCCCTCTTCCTCCAGGTCCCCCTTCCCCGCAACTT | |
| | | CCCACGAGTGCCAGGTGCCGCGAGCGCCGAGTTCCGCGCA | |
| | | TTGGAAAGAAGCGACCGCGGCGGCTGGAACCCTGATTGCT | |
| | | GTCCTTCAACGTGTTCATTATGAAGTTATTAGTAATACTT | |
| | | TTGTTTTCTGGACTTATAACTGGTTTTAGAAGTGACTCTT | |
| | | CCTCTAGTTTGCCACCTAAGTTACTACTAGTATCCTTTGA | |
| | | TGGCTTCAGAGCTGATTATCTGAAGAACTATGAATTTCCT | |
| | | CATCTCCAGAATTTTATCAAAGAAGGTGTTTTGGTAGAGC | |
| | | ATGTTAAAAATGTTTTTATCACAAAAACATTTCCAAACCA | |
| | | CTACAGTATTGTGACAGGCTTGTATGAAGAAAGCCATGGC | |
| | | ATTGTGGCTAATTCCATGTATGATGCAGTCACAAAGAAAC | |
| | | ACTTTTCTGACTCTAATGACAAGGATCCTTTTTGGTGGAA | |
| | | TGAGGCAGTACCTATTTGGGTGACCAATCAGCTTCAGGAA | |
| | | AACAGATCAAGTGCTGCTGCTATGTGGCCTGGTACTGATG | |
| | | TACCCATTCACGATACCATCTCTTCCTATTTTATGAATTA | |
| | | CAACTCCTCAGTGTCATTTGAGGAAAGACTAAATAATATT | |
| | | ACTATGTGGCTAAACAATTCGAACCCACCAGTCACCTTTG | |
| | | CAACACTATATTGGGAAGAACCAGATGCAAGTGGCCACAA | |
| | | ATACGGACCTGAAGATAAAGAAAACATGAGCAGAGTGTTG | |
| | | AAAAAAATAGATGATCTTATCGGTGACTTAGTCCAAAGAC | |
| | | TCAAGATGTTAGGGCTATGGGAAAATCTTAATGTGATCAT | |
| | | TACAAGTGATCATGGGATGACCCAGTGTTCTCAGGACAGA | |
| | | CTGATAAACCTGGATTCCTGCATCGATCATTCATACTACA | |
| | | CTCTTATAGATTTGAGCCCAGTTGCTGCAATACTTCCCAA | |
| | | AATAAATAGAACAGAGGTTTATAACAAACTGAAAAACTGT | |
| | | AGCCCTCATATGAATGTTTATCTCAAAGAAGACATTCCTA | |
| | | ACAGATTTTATTACCAACATAATGATCGAATTCAGCCCAT | |
| | | TATTTTGGTTGCCGATGAAGGCTGGACAATTGTGCTAAAT | |
| | | GAATCATCACAAAAATTAGGTGACCATGGTTATGATAATT | |
| | | CTTTGCCTAGTATGCATCCATTTCTAGCTGCCCACGGACC | |
| | | TGCATTTCACAAAGGCTACAAGCATAGCACAATTAACATT | |
| | | GTGGATATTTATCCAATGATGTGCCACATCCTGGGATTAA | |
| | | AACCACATCCCAATAATGGGACCTTTGGTCATACTAAGTG | |
| | | CTTGTTAGTTGACCAGTGGTGCATTAATCTCCCAGAAGCC | |
| | | ATCGCGATTGTTATCGGTTCACTCTTGGTGTTAACCATGC | |
| | | TAACATGCCTCATAATAATCATGCAGAATAGACTTTCTGT | |
| | | ACCTCGTCCATTTTCTCGACTTCAGCTACAAGAAGATGAT | |
| | | GATGATCCTTTAATTGGGTGACATGTGCTAGGGCTTATAC | |
| | | AAAGTGTCTTTGATTAATCACAAAACTAAGAATACATCCA | |
| | | AAGAATAGTGTTGTAACTATGAAAAAGAATACTTTGAAAG | |
| | | ACAAAGAACTTAGACTAAGCATGTTAAAATTATTACTTTG | |
| | | TTTTCCTTGTGTTTTGTTTCGGTGCATTTGCTAATAAGAT | |
| | | AACGCTGACCATAGTAAAATTGTTAGTAAATCATTAGGTA | |
| | | ACATCTTGTGGTAGGAAATCATTAGGTAACATCAATCCTA | |
| | | ACTAGAAATACTAAAAATGGCTTTTGAGAAAAATACTTCC | |
| | | TCTGCTTGTATTTTGCGATGAAGATGTGATACATCTTTAA | |
| | | ATGAAAATATACCAAAATTTAGTAGGCATGTTTTTCTAAT | |
| | | AAATTTATATATTTGTAAAGAAAACAACAGAAATCTTTAT | |
| | | GCAATTTGTGAATTTTGTATATTAGGGAGGAAAAGCTTCC | |
| | | TATATTTTTATATTTACCTTTAATTAGTTTGTATCTCAAG | |
| | | TACCCTCTTGAGGTAGGAAATGCTCTGTGATGGTAAATAA | |
| | | AATTGGAGCAGACAGAAAAGATATAGCAAATGAAGAAATA | |
| | | TTTTAAGGAAACCTATTTGAAAAAAAAAGCAAAGACCATT | |
| | | TGATAAAAGCCTGAGTTGTCACCATTATGTCTTAAGCTGT | |
| | | TAGTCTTAAAGATTATTGTTAAAAAATTCAGAAGAAAAGA | |
| | | GAGACAAGTGCTCTTCTCTCTATCTATGCTTAATGCCTTT | |
| | | ATGTAAGTTACTTAGTTGTTTGCGTGTGCCTGTGCAAGTG | |
| | | TGTTTGTGTGTGGTTGTGTGGACATTATGTGATTTACTAT | |
| | | ATAAGGAGGTCAGAGATGGACTGTGGCCAGGCTTCCACAT | |
| | | TCCTGAAGCACACAGATCTCAGGAAAGGTTATTTTTGCAC | |
| | | TTCATATTTGTTTACTTTCTCCTAACTCACAAGTTAAAAT | |
| | | CATAACTTAATTTCATTAACTTTTATCATTTAACTCTCTC | |
| | | ATGTTTGTTGTAACCTGAGGTATCCAAATGCTACAGAAAA | |
| | | ATTTATGACCCAAATACAAATCTCAATTTGACTGGGACAG | |
| | | AATGAGGAATGGAGATTTTTGTATTTATCTTTGGGACTTT | |
| | | ATGCCTTACTTTTTAGGCTATAGAATAGTTAAGAAATTTT | |
| | | AAACAAAATTTAGTATCTTTTGGTCTTTCACACCATTCAT | |
| | | ATGTTAAGTGGCAGAATAGCCTTAGTGCTACCTCCACTTT | |
| | | TTTCTCCAGTATTTGCATCACAGAAATAATCCCTCTGTTT | |
| | | AACATGTTTGTTCAGAGCCAAGGGTTTATTGTGAAGAACT | |
| | | GTCATCCTGCCTTTGCTAGCTGGTACCTTCTAGTAATCAA | |
| | | AATTAATATGAAGAAACTAGGTTGTGACAGACTAGATTAT | |
| | | ATTTAGTAGGGGAAAAATTGGGCTCAAGAACCATTCATCA | |
| | | GTACGTGAGACAAGCAGTTAATAGTATGATCTTTAAAGTT | |
| | | TTGACAATATAAAATAAACTTGGTAACTGTTTTACAAATA | |
| | | TAAAAGTATAATAAATATGCAGCCCAGTTAAATATTGATT | |
| | | ATCTGTGATGGTAAAGAACAACAGTGGTGCCAGTCATCAA | |
| | | ACATACAGTGCGTCCTATTGAGTCACTGCTAATTTCTTGA | |
| | | GCCTGGTATTTGCTGCCTATTGTATTTGTGGTTGTTGAGA | |
| | | GGCATTTTCAAACCCTGTATAAATAATCCATGCTGTTGGT | |
| | | CATAAGTTAACTGTATTAAGAACAGTAAAATAAATAAAAA | |
| | | CCAATAGTACTAATTTTGCTTTAAAAAAATTTCTAATTTT | |
| | | TTTCACATAAAACAATTATCCTAAAGGTTAATAGTTGATC | |
| | | GAAACAGAATAATAGAAAAATTCTACTTTAATTTCCATTA | |
| | | AAAAGCAAATAGCATTGACACATTTAAAGCTTTTCATTTA | |
| | | AAGTAGTGGATGTTTTTGAAGTATCTAAAATAGTAGCAGA | |
| | | ATATTTTATACTTGGTCCTTGCAATGGTGTGAGTTTTAAT | |
| | | GATTGCATTATCGTGATTGGTGGTTATGAGTTTCAGAAAT | |
| | | CTATACTTGGCATCCAACTCATGAGTGGATTTTATATAGG | |
| | | ATGGAACAGGAAGGTATGTCCTGTCAGTATCTTAACCCTT | |
| | | TCAACAAGACATTTACCTATTTGTCTTTCCTTACGTTCTC | |
| | | AAAATATTAACTCGAATTGTAAATTAAGCAAAAATTTAAA | |
| | | AAGTATATGTTGATGGGACAAGAAGAATAGTATTTATTTA | |
| | | ATAAAACATATATTATATTGAACTATGTGTTAATTCATTT | |
| | | GTATCTTTTAAAAAATTATCACTGTTAAAGCCATTGACTC | |
| | | CTTTAGTACACTGAGAAAAATCTTATAGTAAAACTAGCCT | |
| | | TTCACATTAAGGTTTTGGTGTGTATTTTGTTAAATAACTA | |
| | | ACATGCTGCTCTATTTTCTGGGTGTAGAAAGTATTTGGCT | |
| | | CTAGGAAACATTTACTTGTTTGTGAAAACAATACCCCAAG | |
| | | GTAATAGGAAAAGTTTGAGTTAAGTGTTTTTAATTCAGTC | |
| | | AGTGAATTCAGAATAAGTACATTCATGTATAACATAGGGA | |
| | | CAGTTCTGCTGCTGTTATTTATATGCAATTCTTCTGGTAA | |
| | | ATAGCAATAGAATAAAACATATTTCAATGTTTGTGTATAG | |
| | | GTTTTATATTATTATTCCACTAGGAATGGCATAAGAATTT | |
| | | ATAGATAAATTCTTGTAACATTAAAGGATTAAAATGTTTT | |
| | | TACATTGTTTTTGGGTGTCTCCTTCTTGTGCCCATATCTG | |
| | | ATAAGCTTTATGGATTATTGCATTTAATTCCTTTTATTTG | |
| | | GAGGGTTTTACTTCCTTGTTAACATATAAAGTTATAAATG | |
| | | AAGGACAAGGAGGAGATGGAAAATGTGTATTTATTGTTAA | |
| | | TTCTTAAAATAGTGTGTAAATAAAATAACATCAGTGTGCT | |
| | | TTAAAGAAATGTGTATGTAGTGCCTTAATTTAAATTAAAA | |
| | | TATTTTTGACTGTTACTTGAGTTCAGAATTAATGACTTTG | |
| | | TTCATGATTTTTAAAATGTGTGTGAATAAAATCTACCAAA | |
| | | AAATTCTTACTGTAATTATTAAATATAAAGTTCAGTGTCA | |
| | | AAAAAAAAAAAAAAAAA | |
| FAM131A | NM_001171093.1 | ACCGGCCCGGTTCCCTCTCCGGGGAGCGGCGGCGGACGCG | 13 |
| | | CGGCTCCCACCCCTCCCCTCTCACGGGCTCTCCCCTCCCC | |
| | | AGTGTGGCCGCGACCCTACCCTCTGCAAGGCGATGGCCCG | |
| | | CGCCCCGAGCGCAGGCTAGCGTGCCTGGGTGCCCGGCCAT | |
| | | GGGCTGTATCGGCTCTCGGAGCCCGGCGGGTCAGGCATTT | |
| | | CTGGGGACCAACAGCTGGCCGAGGCTCAGGGATAGAGACG | |
| | | GCTGCTCCAGCTAAAGGTGAATGTTGGAGACACAGTCGCG | |
| | | ATGCTGCCCAAGTCCCGGCGAGCCCTAACTATCCAGGAGA | |
| | | TCGCTGCGCTGGCCAGGTCCTCCCTGCATGGTATTTCCCA | |
| | | GGTGGTGAAGGACCACGTGACCAAGCCTACCGCCATGGCC | |
| | | CAGGGCCGAGTGGCTCACCTCATTGAGTGGAAGGGCTGGA | |
| | | GCAAGCCGAGTGACTCACCTGCTGCCCTGGAATCAGCCTT | |
| | | TTCCTCCTATTCAGACCTCAGCGAGGGCGAACAAGAGGCT | |
| | | CGCTTTGCAGCAGGAGTGGCTGAGCAGTTTGCCATCGCGG | |
| | | AAGCCAAGCTCCGAGCATGGTCTTCGGTGGATGGCGAGGA | |
| | | CTCCACTGATGACTCCTATGATGAGGACTTTGCTGGGGGA | |
| | | ATGGACACAGACATGGCTGGGCAGCTGCCCCTGGGGCCGC | |
| | | ACCTCCAGGACCTGTTCACCGGCCACCGGTTCTCCCGGCC | |
| | | TGTGCGCCAGGGCTCCGTGGAGCCTGAGAGCGACTGCTCA | |
| | | CAGACCGTGTCCCCAGACACCCTGTGCTCTAGTCTGTGCA | |
| | | GCCTGGAGGATGGGTTGTTGGGCTCCCCGGCCCGGCTGGC | |
| | | CTCCCAGCTGCTGGGCGATGAGCTGCTTCTCGCCAAACTG | |
| | | CCCCCCAGCCGGGAAAGTGCCTTCCGCAGCCTGGGCCCAC | |
| | | TGGAGGCCCAGGACTCACTCTACAACTCGCCCCTCACAGA | |
| | | GTCCTGCCTTTCCCCCGCGGAGGAGGAGCCAGCCCCCTGC | |
| | | AAGGACTGCCAGCCACTCTGCCCACCACTAACGGGCAGCT | |
| | | GGGAACGGCAGCGGCAAGCCTCTGACCTGGCCTCTTCTGG | |
| | | GGTGGTGTCCTTAGATGAGGATGAGGCAGAGCCAGAGGAA | |
| | | CAGTGACCCACATCATGCCTGGCAGTGGCATGCATCCCCC | |
| | | GGCTGCTGCCAGGGGCAGAGCCTCTGTGCCCAAGTGTGGG | |
| | | CTCAAGGCTCCCAGCAGAGCTCCACAGCCTAGAGGGCTCC | |
| | | TGGGAGCGCTCGCTTCTCCGTTGTGTGTTTTGCATGAAAG | |
| | | TGTTTGGAGAGGAGGCAGGGGCTGGGCTGGGGGCGCATGT | |
| | | CCTGCCCCCACTCCCGGGGCTTGCCGGGGGTTGCCCGGGG | |
| | | CCTCTGGGGCATGGCTACAGCTGTGGCAGACAGTGATGTT | |
| | | CATGTTCTTAAAATGCCACACACACATTTCCTCCTCGGAT | |
| | | AATGTGAACCACTAAGGGGGTTGTGACTGGGCTGTGTGAG | |
| | | GGTGGGGTGGGAGGGGGCCCAGCAACCCCCCACCCTCCCC | |
| | | ATGCCTCTCTCTTCTCTGCTTTTCTTCTCACTTCCGAGTC | |
| | | CATGTGCAGTGCTTGATAGAATCACCCCCACCTGGAGGGG | |
| | | CTGGCTCCTGCCCTCCCGGAGCCTATGGGTTGAGCCGTCC | |
| | | CTCAAGGGCCCCTGCCCAGCTGGGCTCGTGCTGTGCTTCA | |
| | | TTCACCTCTCCATCGTCTCTAAATCTTCCTCTTTTTTCCT | |
| | | AAAGACAGAAGGTTTTTGGTCTGTTTTTTCAGTCGGATCT | |
| | | TCTCTTCTCTGGGAGGCTTTGGAATGATGAAAGCATGTAC | |
| | | CCTCCACCCTTTTCCTGGCCCCCTAATGGGGCCTGGGCCC | |
| | | TTTCCCAACCCCTCCTAGGATGTGCGGGCAGTGTGCTGGC | |
| | | GCCTCACAGCCAGCCGGGCTGCCCATTCACGCAGAGCTCT | |
| | | CTGAGCGGGAGGTGGAAGAAAGGATGGCTCTGGTTGCCAC | |
| | | AGAGCTGGGACTTCATGTTCTTCTAGAGAGGGCCACAAGA | |
| | | GGGCCACAGGGGTGGCCGGGAGTTGTCAGCTGATGCCTGC | |
| | | TGAGAGGCAGGAATTGTGCCAGTGAGTGACAGTCATGAGG | |
| | | GAGTGTCTCTTCTTGGGGAGGAAAGAAGGTAGAGCCTTTC | |
| | | TGTCTGAATGAAAGGCCAAGGCTACAGTACAGGGCCCCAC | |
| | | CCCAGCCAGGGTGTTAATGCCCACGTAGTGGAGGCCTCTG | |
| | | GCAGATCCTGCATTCCAAGGTCACTGGACTGTACGTTTTT | |
| | | ATGGTTGTGGGAAGGGTGGGTGGCTTTAGAATTAAGGGCC | |
| | | TTGTAGGCTTTGGCAGGTAAGAGGGCCCAAGGTAAGAACG | |
| | | AGAGCCAACGGGCACAAGCATTCTATATATAAGTGGCTCA | |
| | | TTAGGTGTTTATTTTGTTCTATTTAAGAATTTGTTTTATT | |
| | | AAATTAATATAAAAATCTTTGTAAATCTCTAAAAAAAAAA | |
| | | AAAAAAAA | |
| FLJ10357 | NM_018071.4 | GGAGCGGGCCGAGCCGCCACCGCGGCCGGAGCTGTCCCTT | 14 |
| | | AGCCAGACCCGGCGAGACACGAGCGGCGGGAGGGAGGCGG | |
| | | TGGCGCGCCCGGCCCCGCCCGCCCGACCAAGCGTCGGACG | |
| | | CGGCCCGGCGCCGAGCCATGGAGCCTGAGCCAGTGGAGGA | |
| | | CTGTGTGCAGAGCACTCTCGCCGCCCTGTATCCACCCTTT | |
| | | GAGGCAACAGCCCCCACCCTGTTGGGCCAGGTGTTCCAGG | |
| | | TGGTGGAGAGGACTTATCGGGAGGACGCACTGAGGTACAC | |
| | | GCTGGACTTCCTGGTACCAGCCAAGCACCTGCTTGCCAAG | |
| | | GTCCAGCAGGAAGCCTGTGCCCAATACAGTGGATTCCTCT | |
| | | TCTTCCATGAGGGGTGGCCGCTCTGCCTGCATGAACAGGT | |
| | | GGTGGTGCAGCTAGCAGCCCTACCCTGGCAACTGCTGCGC | |
| | | CCAGGAGACTTCTATCTGCAGGTGGTGCCCTCAGCTGCCC | |
| | | AAGCACCCCGACTAGCACTCAAGTGTCTGGCCCCTGGGGG | |
| | | TGGGCGGGTGCAGGAGGTTCCTGTGCCCAATGAGGCTTGT | |
| | | GCCTACCTATTCACACCTGAGTGGCTACAAGGCATCAACA | |
| | | AGGACCGGCCAACAGGTCGCCTCAGTACCTGCCTACTGTC | |
| | | TGCGCCCTCTGGGATTCAGCGGCTGCCCTGGGCTGAGCTC | |
| | | ATCTGTCCACGATTTGTGCACAAAGAGGGCCTCATGGTTG | |
| | | GACATCAGCCAAGTACACTGCCCCCAGAACTGCCCTCTGG | |
| | | ACCTCCAGGGCTTCCCAGCCCTCCACTTCCTGAGGAGGCG | |
| | | CTGGGTACCCGGAGTCCTGGGGATGGGCACAATGCCCCTG | |
| | | TGGAAGGACCTGAGGGCGAGTATGTGGAGCTGTTAGAGGT | |
| | | GACGCTGCCCGTGAGGGGGAGCCCAACAGATGCTGAAGGC | |
| | | TCCCCAGGCCTCTCCAGAGTCCGGACGGTACCCACCCGCA | |
| | | AGGGCGCTGGAGGGAAGGGCCGCCACCGGAGACACCGGGC | |
| | | GTGGATGCACCAGAAGGGCCTGGGGCCTCGGGGCCAGGAT | |
| | | GGAGCACGCCCACCCGGCGAGGGGAGCAGCACCGGAGCCT | |
| | | CCCCTGAGTCTCCCCCAGGAGCTGAGGCTGTCCCAGAGGC | |
| | | AGCAGTCTTGGAGGTGTCTGAGCCCCCAGCAGAGGCTGTG | |
| | | GGAGAAGCCTCCGGATCTTGCCCCCTGAGGCCAGGGGAGC | |
| | | TTAGAGGAGGAGGAGGAGGAGGCCAGGGGGCTGAAGGACC | |
| | | ACCTGGTACCCCTCGGAGAACAGGCAAAGGAAACAGAAGA | |
| | | AAGAAGCGAGCTGCAGGTCGAGGGGCTCTTAGCCGAGGAG | |
| | | GGGACAGTGCCCCACTGAGCCCTGGGGACAAGGAAGATGC | |
| | | CAGCCACCAAGAAGCCCTTGGCAATCTGCCCTCACCAAGT | |
| | | GAGCACAAGCTTCCAGAATGCCACCTGGTTAAGGAGGAAT | |
| | | ATGAAGGCTCAGGGAAGCCAGAATCTGAGCCAAAAGAGCT | |
| | | CAAAACAGCAGGCGAGAAAGAGCCTCAGCTCTCTGAAGCC | |
| | | TGTGGGCCTACAGAAGAGGGGGCCGGAGAGAGAGAGCTGG | |
| | | AGGGGCCAGGCCTGCTGTGTATGGCAGGACACACAGGCCC | |
| | | AGAAGGCCCCCTGTCTGACACTCCAACACCTCCGCTGGAG | |
| | | ACTGTGCAGGAAGGAAAAGGGGACAACATTCCAGAAGAGG | |
| | | CCCTTGCAGTCTCCGTCTCTGATCACCCTGATGTAGCTTG | |
| | | GGACTTGATGGCATCTGGATTCCTCATCCTGACGGGAGGG | |
| | | GTGGACCAGAGTGGGCGAGCTCTGCTGACCATTACCCCAC | |
| | | CGTGCCCTCCTGAGGAGCCCCCACCCTCCCGAGACACGCT | |
| | | GAACACAACTCTTCATTACCTCCACTCACTGCTCAGGCCT | |
| | | GATCTACAGACACTGGGGCTGTCCGTCCTGCTGGACCTTC | |
| | | GTCAGGCACCTCCACTGCCTCCAGCACTCATTCCTGCCTT | |
| | | GAGCCAACTTCAGGACTCAGGAGATCCTCCCCTTGTTCAG | |
| | | CGGCTGCTGATTCTCATTCATGATGACCTTCCAACTGAAC | |
| | | TCTGTGGATTTCAGGGTGCTGAGGTGCTGTCAGAGAATGA | |
| | | TCTGAAAAGAGTGGCCAAGCCAGAGGAGCTGCAGTGGGAG | |
| | | TTAGGAGGTCACAGGGACCCCTCTCCCAGTCACTGGGTAG | |
| | | AGATACACCAGGAAGTGGTAAGGCTATGTCGCCTGTGCCA | |
| | | AGGTGTGCTGGGCTCGGTACGGCAGGCCATTGAGGAGCTG | |
| | | GAGGGAGCAGCAGAGCCAGAGGAAGAGGAGGCAGTGGGAA | |
| | | TGCCCAAGCCACTGCAGAAGGTGCTGGCAGATCCCCGGCT | |
| | | GACGGCACTGCAGAGGGATGGGGGGGCCATCCTGATGAGG | |
| | | CTGCGCTCCACTCCCAGCAGCAAGCTGGAGGGCCAAGGCC | |
| | | CAGCTACACTGTATCAGGAAGTGGACGAGGCCATTCACCA | |
| | | GCTTGTGCGCCTCTCCAACCTGCACGTGCAGCAGCAAGAG | |
| | | CAGCGGCAGTGCCTGCGGCGACTCCAGCAGGTGTTGCAGT | |
| | | GGCTCTCGGGCCCAGGGGAGGAGCAGCTGGCAAGCTTTGC | |
| | | TATGCCTGGGGACACCTTGTCTGCCCTGCAGGAGACAGAG | |
| | | CTGCGATTCCGTGCTTTCAGCGCTGAGGTCCAGGAGCGCC | |
| | | TGGCCCAGGCACGGGAGGCCCTGGCTCTGGAGGAGAATGC | |
| | | CACCTCCCAGAAGGTGCTGGATATCTTTGAACAGCGGCTG | |
| | | GAGCAGGTTGAGAGTGGCCTCCATCGGGCCCTGCGGCTAC | |
| | | AGCGCTTCTTCCAGCAGGCACATGAATGGGTGGATGAGGG | |
| | | CTTTGCTCGGCTGGCAGGAGCTGGGCCGGGTCGGGAGGCT | |
| | | GTGCTGGCTGCACTGGCCCTGCGGCGGGCCCCAGAGCCCA | |
| | | GTGCCGGCACCTTCCAGGAGATGCGGGCCCTGGCCCTGGA | |
| | | CCTGGGCAGCCCAGCAGCCCTGCGAGAATGGGGCCGCTGC | |
| | | CAGGCCCGCTGCCAAGAGCTAGAGAGGAGGATCCAGCAAC | |
| | | ACGTGGGAGAGGAGGCGAGCCCACGGGGCTACCGACGACG | |
| | | GCGGGCAGACGGTGCCAGCAGTGGAGGGGCCCAGTGGGGG | |
| | | CCCCGCAGCCCCTCGCCCAGCCTCAGCTCCTTGCTGCTCC | |
| | | CCAGCAGCCCTGGGCCACGGCCAGCCCCATCCCATTGCTC | |
| | | CCTGGCCCCATGTGGAGAGGACTATGAGGAAGAGGGCCCT | |
| | | GAGCTGGCTCCAGAAGCAGAGGGCAGGCCCCCAAGAGCTG | |
| | | TGCTGATCCGAGGCCTGGAGGTCACCAGCACTGAGGTGGT | |
| | | AGACAGGACGTGCTCACCACGGGAACACGTGCTGCTGGGC | |
| | | CGGGCTAGGGGGCCAGACGGACCCTGGGGAGTAGGCACCC | |
| | | CCCGGATGGAGCGCAAGCGAAGCATCAGTGCCCAGCAGCG | |
| | | GCTGGTGTCTGAGCTGATTGCCTGTGAACAAGATTACGTG | |
| | | GCCACCTTGAGTGAGCCAGTGCCACCCCCTGGGCCTGAGC | |
| | | TGACGCCTGAACTTCGGGGCACCTGGGCTGCTGCCCTGAG | |
| | | TGCCCGGGAAAGGCTTCGCAGCTTCCACCGGACACACTTT | |
| | | CTGCGGGAGCTTCAGGGCTGCGCCACCCACCCCCTACGCA | |
| | | TTGGGGCCTGCTTCCTTCGCCACGGGGACCAGTTCAGCCT | |
| | | TTATGCACAGTACGTGAAGCACCGACACAAACTGGAGAAT | |
| | | GGTCTGGCTGCGCTCAGTCCCTTAAGCAAGGGCTCCATGG | |
| | | AGGCTGGCCCTTACCTGCCCCGAGCCCTGCAGCAGCCTCT | |
| | | GGAACAGCTGACTCGGTATGGGCGGCTCCTGGAGGAGCTC | |
| | | CTGAGGGAAGCTGGGCCTGAGCTCAGTTCTGAGTGCCGGG | |
| | | CCCTTGGGGCTGCTGTACAGCTGCTCCGGGAACAAGAGGC | |
| | | CCGTGGCAGAGACCTGCTGGCCGTGGAGGCGGTGCGTGGC | |
| | | TGTGAGATAGATCTGAAGGAGCAGGGACAGCTCTTGCATC | |
| | | GAGACCCCTTCACTGTCATCTGTGGCCGAAAGAAGTGCCT | |
| | | TCGCCATGTCTTTCTCTTCGAGCATCTCCTCCTGTTCAGC | |
| | | AAGCTCAAGGGCCCTGAAGGGGGGTCAGAGATGTTTGTTT | |
| | | ACAAGCAGGCCTTTAAGACTGCTGATATGGGGCTGACAGA | |
| | | AAACATCGGGGACAGCGGACTCTGCTTTGAGTTGTGGTTT | |
| | | CGGCGGCGGCGTGCACGAGAGGCATACACTCTGCAGGCAA | |
| | | CCTCACCAGAGATCAAACTCAAGTGGACAAGTTCTATTGC | |
| | | CCAGCTGCTGTGGAGACAGGCAGCCCACAACAAGGAGCTC | |
| | | CGAGTGCAGCAGATGGTGTCCATGGGCATTGGGAATAAAC | |
| | | CCTTCCTGGACATCAAAGCCCTTGGGGAGCGGACGCTGAG | |
| | | TGCCCTGCTCACTGGAAGAGCCGCCCGCACCCGGGCCTCC | |
| | | GTGGCCGTGTCATCCTTTGAGCATGCCGGCCCCTCCCTTC | |
| | | CCGGCCTTTCGCCGGGAGCCTGCTCCCTGCCTGCCCGCGT | |
| | | CGAGGAGGAGGCCTGGGATCTGGACGTCAAGCAAATTTCC | |
| | | CTGGCCCCAGAAACACTTGACTCTTCTGGAGATGTGTCCC | |
| | | CAGGACCAAGAAACAGCCCCAGCCTGCAACCCCCCCACCC | |
| | | TGGGAGCAGCACTCCCACCCTGGCCAGTCGAGGGATCTTA | |
| | | GGGCTATCCCGACAGAGTCATGCTCGAGCCCTGAGTGACC | |
| | | CCACCACGCCTCTGTGACCTGGAGAAGATCCAGAACTTGC | |
| | | GTGCAGCTTCTCCTCTCAGCACACTTTGGGCTGGGATGGC | |
| | | AGTGGGGCATAATGGAGCCCTGGGCGATCGCTGAATTTCT | |
| | | TCCCTCTGCTTCCTGGACACAGAGGAGGTCTAACGACCAG | |
| | | AGTATTGCCCTGCCACCACTATCTCTAGTCTCCCTAGCTT | |
| | | GGTGCCTTCTCCTGCAGGAGTCAGAGCAGCCACATTGCTT | |
| | | GCCTTCATACCCTGGAGGTGGGGAAGTTATCCCTCTTCCG | |
| | | GTGCTTTCCCATCCTGGGCCACTGTATCCAGGACATCACT | |
| | | CCCATGCCAGCCCTCCCTGGCAGCCCATGTTCTCCTCTTT | |
| | | TCTCACCCCCTGACTTTCCCTGAGAAGAATCATCTCTGCC | |
| | | AGGTCAACTGGAGTCCCTGGTGACTCCATTCTGAGGTGTC | |
| | | ACAAGCAATGAAGCTATGCAAACAATAGGAGGGTGTGACA | |
| | | GGGGAACCGTAGACTTTATATATGTAATTACTGTTATTAT | |
| | | AATACTATTGTTATATTAAATGTATTTACTCACACTTTGC | |
| | | CTCTAAGGAGCTAGAGTAGTCCTCTGGATTAAGGTGATAA | |
| | | ATAACTTGAGCACTTTCCCTCAACCAGCCCTTAACTAGAA | |
| | | CACAGAAAATAAAACCAAGACTGGAAGGTCCCCTCTACCC | |
| | | CTCCCAGGCCCAGAGCTAGCTGACTGTGTATGAGCCTGGG | |
| | | AGAATGTGTCTCCTCCACAGTGGCTCCCAGAGGTTCCACA | |
| | | CACTCTCTGAAGCTCCTTCTCCCACACTGCACCTACTCCT | |
| | | TGAGGCTGAACTGGTCACAGACAAACTGGGATCCAGCACA | |
| | | GTCCAGCAGTTCTCAAAATGAGGTCCTCAGGCCACAGTGC | |
| | | GTGAGAACTTGCTTGGCTGTTTGTTAAATGCTAATTCTTG | |
| | | GGCCCCATCAGAGCTACTGCATCGAAACCTGGGGGTAAAA | |
| | | CCCAATATTCTGCATTTCTTATCAAACTCTTTGGGTGATA | |
| | | ACTAAGTGTCTGAAGAGGTGACTATTTCCTGACAGAAGGA | |
| | | CCCAAAGAGGGAAGCAGGACATAGGTAGGCAGACAGACAC | |
| | | AGGGCCCTGTGCCTCAAGACACCTGTTTATTGGGGACACG | |
| | | ACTCTGCAATAGGGATGACAGGAATCGTACCAAAAATAGC | |
| | | GACGTCTACAGGGCCCCTGATGGGGCTAGAAGGGTACAGT | |
| | | GCCCCCCACCCTCACCCCTTGTACAAAAATAAACTCTCAC | |
| | | GCCTATGGACCAGCAAAAAAAAAAAAAA | |
| FZD7 | NM_003507.1 | CTCTCCCAACCGCCTCGTCGCACTCCTCAGGCTGAGAGCA | 15 |
| | | CCGCTGCACTCGCGGCCGGCGATGCGGGACCCCGGCGCGG | |
| | | CCGCTCCGCTTTCGTCCCTGGGCCTCTGTGCCCTGGTGCT | |
| | | GGCGCTGCTGGGCGCACTGTCCGCGGGCGCCGGGGCGCAG | |
| | | CCGTACCACGGAGAGAAGGGCATCTCCGTGCCGGACCACG | |
| | | GCTTCTGCCAGCCCATCTCCATCCCGCTGTGCACGGACAT | |
| | | CGCCTACAACCAGACCATCCTGCCCAACCTGCTGGGCCAC | |
| | | ACGAACCAAGAGGACGCGGGCCTCGAGGTGCACCAGTTCT | |
| | | ACCCGCTGGTGAAGGTGCAGTGTTCTCCCGAACTCCGCTT | |
| | | TTTCTTATGCTCCATGTATGCGCCCGTGTGCACCGTGCTC | |
| | | GATCAGGCCATCCCGCCGTGTCGTTCTCTGTGCGAGCGCG | |
| | | CCCGCCAGGGCTGCGAGGCGCTCATGAACAAGTTCGGCTT | |
| | | CCAGTGGCCCGAGCGGCTGCGCTGCGAGAACTTCCCGGTG | |
| | | CACGGTGCGGGCGAGATCTGCGTGGGCCAGAACACGTCGG | |
| | | ACGGCTCCGGGGGCCCAGGCGGCGGCCCCACTGCCTACCC | |
| | | TACCGCGCCCTACCTGCCGGACCTGCCCTTCACCGCGCTG | |
| | | CCCCCGGGGGCCTCAGATGGCAGGGGGCGTCCCGCCTTCC | |
| | | CCTTCTCATGCCCCCGTCAGCTCAAGGTGCCCCCGTACCT | |
| | | GGGCTACCGCTTCCTGGGTGAGCGCGATTGTGGCGCCCCG | |
| | | TGCGAACCGGGCCGTGCCAACGGCCTGATGTACTTTAAGG | |
| | | AGGAGGAGAGGCGCTTCGCCCGCCTCTGGGTGGGCGTGTG | |
| | | GTCCGTGCTGTGCTGCGCCTCGACGCTCTTTACCGTTCTC | |
| | | ACCTACCTGGTGGACATGCGGCGCTTCAGCTACCCAGAGC | |
| | | GGCCCATCATCTTCCTGTCGGGCTGCTACTTCATGGTGGC | |
| | | CGTGGCGCACGTGGCCGGCTTCCTTCTAGAGGACCGCGCC | |
| | | GTGTGCGTGGAGCGCTTCTCGGACGATGGCTACCGCACGG | |
| | | TGGCGCAGGGCACCAAGAAGGAGGGCTGCACCATCCTCTT | |
| | | CATGGTGCTCTACTTCTTCGGCATGGCCAGCTCCATCTGG | |
| | | TGGGTCATTCTGTCTCTCACTTGGTTCCTGGCGGCCGGCA | |
| | | TGAAGTGGGGCCACGAGGCCATCGAGGCCAACTCGCAGTA | |
| | | CTTCCACCTGGCCGCGTGGGCCGTGCCCGCCGTCAAGACC | |
| | | ATCACTATCCTGGCCATGGGCCAGGTAGACGGGGACCTGC | |
| | | TGAGCGGGGTGTGCTACGTTGGCCTCTCCAGTGTGGACGC | |
| | | GCTGCGGGGCTTCGTGCTGGCGCCTCTGTTCGTCTACCTC | |
| | | TTCATAGGCACGTCCTTCTTGCTGGCCGGCTTCGTGTCCC | |
| | | TCTTCCGTATCCGCACCATCATGAAACACGACGGCACCAA | |
| | | GACCGAGAAGCTGGAGAAGCTCATGGTGCGCATCGGCGTC | |
| | | TTCAGCGTGCTCTACACAGTGCCCGCCACCATCGTCCTGG | |
| | | CCTGCTACTTCTACGAGCAGGCCTTCCGCGAGCACTGGGA | |
| | | GCGCACCTGGCTCCTGCAGACGTGCAAGAGCTATGCCGTG | |
| | | CCCTGCCCGCCCGGCCACTTCCCGCCCATGAGCCCCGACT | |
| | | TCACCGTCTTCATGATCAAGTACCTGATGACCATGATCGT | |
| | | CGGCATCACCACTGGCTTCTGGATCTGGTCGGGCAAGACC | |
| | | CTGCAGTCGTGGCGCCGCTTCTACCACAGACTTAGCCACA | |
| | | GCAGCAAGGGGGAGACTGCGGTATGAGCCCCGGCCCCTCC | |
| | | CCACCTTTCCCACCCCAGCCCTCTTGCAAGAGGAGAGGCA | |
| | | CGGTAGGGAAAAGAACTGCTGGGTGGGGGCCTGTTTCTGT | |
| | | AACTTTCTCCCCCTCTACTGAGAAGTGACCTGGAAGTGAG | |
| | | AAGTTCTTTGCAGATTTGGGGCGAGGGGTGATTTGGAAAA | |
| | | GAAGACCTGGGTGGAAAGCGGTTTGGATGAAAAGATTTCA | |
| | | GGCAAAGACTTGCAGGAAGATGATGATAACGGCGATGTGA | |
| | | ATCGTCAAAGGTACGGGCCAGCTTGTGCCTAATAGAAGGT | |
| | | TGAGACCAGCAGAGACTGCTGTGAGTTTCTCCCGGCTCCG | |
| | | AGGCTGAACGGGGACTGTGAGCGATCCCCCTGCTGCAGGG | |
| | | CGAGTGGCCTGTCCAGACCCCTGTGAGGCCCCGGGAAAGG | |
| | | TACAGCCCTGTCTGCGGTGGCTGCTTTGTTGGAAAGAGGG | |
| | | AGGGCCTCCTGCGGTGTGCTTGTCAAGCAGTGGTCAAACC | |
| | | ATAATCTCTTTTCACTGGGGCCAAACTGGAGCCCAGATGG | |
| | | GTTAATTTCCAGGGTCAGACATTACGGTCTCTCCTCCCCT | |
| | | GCCCCCTCCCGCCTGTTTTTCCTCCCGTACTGCTTTCAGG | |
| | | TCTTGTAAAATAAGCATTTGGAAGTCTTGGGAGGCCTGCC | |
| | | TGCTAGAATCCTAATGTGAGGATGCAAAAGAAATGATGAT | |
| | | AACATTTTGAGATAAGGCCAAGGAGACGTGGAGTAGGTAT | |
| | | TTTTGCTACTTTTTCATTTTCTGGGGAAGGCAGGAGGCAG | |
| | | AAAGACGGGTGTTTTATTTGGTCTAATACCCTGAAAAGAA | |
| | | GTGATGACTTGTTGCTTTTCAAAACAGGAATGCATTTTTC | |
| | | CCCTTGTCTTTGTTGTAAGAGACAAAAGAGGAAACAAAAG | |
| | | TGTCTCCCTGTGGAAAGGCATAACTGTGACGAAAGCAACT | |
| | | TTTATAGGCAAAGCAGCGCAAATCTGAGGTTTCCCGTTGG | |
| | | TTGTTAATTTGGTTGAGATAAACATTCCTTTTTAAGGAAA | |
| | | AGTGAAGAGCAGTGTGCTGTCACACACCGTTAAGCCAGAG | |
| | | GTTCTGACTTCGCTAAAGGAAATGTAAGAGGTTTTGTTGT | |
| | | CTGTTTTAAATAAATTTAATTCGGAACACATGATCCAACA | |
| | | GACTATGTTAAAATATTCAGGGAAATCTCTCCCTTCATTT | |
| | | ACTTTTTCTTGCTATAAGCCTATATTTAGGTTTCTTTTCT | |
| | | ATTTTTTTCTCCCATTTGGATCCTTTGAGGTAAAAAAACA | |
| | | TAATGTCTTCAGCCTCATAATAAAGGAAAGTTAATTAAAA | |
| | | AAAAAAAGCAAAGAGCCATTTTGTCCTGTTTTCTTGGTTC | |
| | | CATCAATCTGTTTATTAAACATCATCCATATGCTGACCCT | |
| | | GTCTCTGTGTGGTTGGGTTGGGAGGCGATCAGCAGATACC | |
| | | ATAGTGAACGAAGAGGAAGGTTTGAACCATGGGCCCCATC | |
| | | TTTAAAGAAAGTCATTAAAAGAAGGTAAACTTCAAAGTGA | |
| | | TTCTGGAGTTCTTTGAAATGTGCTGGAAGACTTAAATTTA | |
| | | TTAATCTTAAATCATGTACTTTTTTTCTGTAATAGAACTC | |
| | | GGATTCTTTTGCATGATGGGGTAAAGCTTAGCAGAGAATC | |
| | | ATGGGAGCTAACCTTTATCCCACCTTTGACACTACCCTCC | |
| | | AATCTTGCAACACTATCCTGTTTCTCAGAACAGTTTTTAA | |
| | | ATGCCAATCATAGAGGGTACTGTAAAGTGTACAAGTTACT | |
| | | TTATATATGTAATGTTCACTTGAGTGGAACTGCTTTTTAC | |
| | | ATTAAAGTTAAAATCGATCTTGTGTTTCTTCAACCTTCAA | |
| | | AACTATCTCATCTGTCAGATTTTTAAAACTCCAACACAGG | |
| | | TTTTGGCATCTTTTGTGCTGTATCTTTTAAGTGCATGTGA | |
| | | AATTTGTAAAATAGAGATAAGTACAGTATGTATATTTTGT | |
| | | AAATCTCCCATTTTTGTAAGAAAATATATATTGTATTTAT | |
| | | ACATTTTTACTTTGGATTTTTGTTTTGTTGGCTTTAAAGG | |
| | | TCTACCCCACTTTATCACATGTACAGATCACAAATAAATT | |
| | | TTTTTAAATAC | |
| GLT8D1 | NM_001010983.2 | GACGGGCCGGTACAGCCCGTGTCCCCGCCCCGCGCCATCG | 16 |
| | | CTAGGCGACGTGCGCTTTTGCCGCGCCGTGCTGCCCGCGA | |
| | | GGGCAGCTGAGGTGGTGGTGGCGGCCGCCTTGTCGAGGCA | |
| | | TCGCGCGCCCGTGAAGTGTTCGCCGTCAGTGCTGTTGGGT | |
| | | GCCTGGAGCCGCGTCCCCCGTCCCGAAAACTGTCCTTGAC | |
| | | AGTACTTGCGCGGCCCAACGGCCGCCGGCGCCCCCGCGTC | |
| | | TCCATGGCGACGGCCTTTTTCCCTGCGAGGACCCCGGCGG | |
| | | CAGGGCTGCCCCGCGGCGCCTGCTTGGCGCGACGCTCTAG | |
| | | CGGTTACCGCTGCGGGCTGGCTGGGCGTAGTGGGGCTGCG | |
| | | CGGCTGCCACGGAGCTAGAGGGCAAGTGTGCTCGGCCCAG | |
| | | CGTGCAGGGAACGCGGGCGGCCAGACAACGGGCTGGGCTC | |
| | | CGGGGCCTGCGGCGCGGGCGCTGAGCTGGCAGGGCGGGTC | |
| | | GGGGCGCGGGCTGCATCCGCATCTCCTCCATCGCCTGCAG | |
| | | TAAGGGCGGCCGCGGCGAGCCTTTGAGGGGAACGACTTGT | |
| | | CGGAGCCCTAACCAGGGGTATCTCTGAGCCTGGTGGGATC | |
| | | CCCGGAGCGTCACATCACTTTCCGATCACTTCAAAGTACA | |
| | | GCAGACCGAGGACACGGTTGTTACCAAGACCAGGCTGTTG | |
| | | CCTTGGAAGAGCCCAGAGCGTGTCAAGGGAGACAGCCACA | |
| | | TCACGCCAGAAATACATGACAGCTGGATTAGCCCTGGGAG | |
| | | AGGGAGGCCCAGATGTGGGAGCTCAGGGGAGGTGCAGCTC | |
| | | AACGTGGAGTTTGGAGGAGGCTACCTTGACCTTTGAATGC | |
| | | CAAGTGGGAGCCAGCCAGATGAAAGGGGTTAAAAACTAAT | |
| | | ATTTATATGACAGAAGAAAAAGATGTCATTCCGTAAAGTA | |
| | | AACATCATCATCTTGGTCCTGGCTGTTGCTCTCTTCTTAC | |
| | | TGGTTTTGCACCATAACTTCCTCAGCTTGAGCAGTTTGTT | |
| | | AAGGAATGAGGTTACAGATTCAGGAATTGTAGGGCCTCAA | |
| | | CCTATAGACTTTGTCCCAAATGCTCTCCGACATGCAGTAG | |
| | | ATGGGAGACAAGAGGAGATTCCTGTGGTCATCGCTGCATC | |
| | | TGAAGACAGGCTTGGGGGGGCCATTGCAGCTATAAACAGC | |
| | | ATTCAGCACAACACTCGCTCCAATGTGATTTTCTACATTG | |
| | | TTACTCTCAACAATACAGCAGACCATCTCCGGTCCTGGCT | |
| | | CAACAGTGATTCCCTGAAAAGCATCAGATACAAAATTGTC | |
| | | AATTTTGACCCTAAACTTTTGGAAGGAAAAGTAAAGGAGG | |
| | | ATCCTGACCAGGGGGAATCCATGAAACCTTTAACCTTTGC | |
| | | AAGGTTCTACTTGCCAATTCTGGTTCCCAGCGCAAAGAAG | |
| | | GCCATATACATGGATGATGATGTAATTGTGCAAGGTGATA | |
| | | TTCTTGCCCTTTACAATACAGCACTGAAGCCAGGACATGC | |
| | | AGCTGCATTTTCAGAAGATTGTGATTCAGCCTCTACTAAA | |
| | | GTTGTCATCCGTGGAGCAGGAAACCAGTACAATTACATTG | |
| | | GCTATCTTGACTATAAAAAGGAAAGAATTCGTAAGCTTTC | |
| | | CATGAAAGCCAGCACTTGCTCATTTAATCCTGGAGTTTTT | |
| | | GTTGCAAACCTGACGGAATGGAAACGACAGAATATAACTA | |
| | | ACCAACTGGAAAAATGGATGAAACTCAATGTAGAAGAGGG | |
| | | ACTGTATAGCAGAACCCTGGCTGGTAGCATCACAACACCT | |
| | | CCTCTGCTTATCGTATTTTATCAACAGCACTCTACCATCG | |
| | | ATCCTATGTGGAATGTCCGCCACCTTGGTTCCAGTGCTGG | |
| | | AAAACGATATTCACCTCAGTTTGTAAAGGCTGCCAAGTTA | |
| | | CTCCATTGGAATGGACATTTGAAGCCATGGGGAAGGACTG | |
| | | CTTCATATACTGATGTTTGGGAAAAATGGTATATTCCAGA | |
| | | CCCAACAGGCAAATTCAACCTAATCCGAAGATATACCGAG | |
| | | ATCTCAAACATAAAGTGAAACAGAATTTGAACTGTAAGCA | |
| | | AGCATTTCTCAGGAAGTCCTGGAAGATAGCATGCGTGGGA | |
| | | AGTAACAGTTGCTAGGCTTCAATGCCTATCGGTAGCAAGC | |
| | | CATGGAAAAAGATGTGTCAGCTAGGTAAAGATGACAAACT | |
| | | GCCCTGTCTGGCAGTCAGCTTCCCAGACAGACTATAGACT | |
| | | ATAAATATGTCTCCATCTGCCTTACCAAGTGTTTTCTTAC | |
| | | TACAATGCTGAATGACTGGAAAGAAGAACTGATATGGCTA | |
| | | GTTCAGCTAGCTGGTACAGATAATTCAAAACTGCTGTTGG | |
| | | TTTTAATTTTGTAACCTGTGGCCTGATCTGTAAATAAAAC | |
| | | TTACATTTTTCAATAGGTAAAAAAAAAAAAAAAA | |
| HDAC9 | NM_001204144.1 | GCAGCGCGCACCGAGCCGGCCGCGCCGCGCCCGCCGCTCT | 17 |
| | | CGCCGCTTTCGCCGCGGTCTCCTCCTCTAGCGCCCGCCGC | |
| | | GGCCGGTAAATCTCGGCTGGAGGAGCAGCGGCGGCCCCCG | |
| | | AGTCAACTTTCATTCCCTTTTTGCTTCTGCCTCACCATTC | |
| | | TCTTCTCCTCCTCGAAAGATGGCTGTTTGGAGAAGGGGGA | |
| | | GAAGTTAAGAGGTCGCCAGCGCGGAGCGAAGGAGGGCGCG | |
| | | ATAGCCTCAGCAGGAGCGGGCGGAGGTTTCTCCTCTGCCA | |
| | | ACCCCTCCTGGACCATTGTCAGCAGTTGAACGACAAAGGC | |
| | | TGTGAATCTGCATCCTAGTCTTAGCAGTCCCTCTGATTCT | |
| | | CATGATGAGCTCACCTGCACAGCCTGACCTCATGTGGAAC | |
| | | CTTGTACCATGGGTGCTATTCTGTGGCTGCTGTAGGATCT | |
| | | TCCCAGATGGGGTGGCTGGACGAGAGCAGCTCTTGGCTCA | |
| | | GCAAAGAATGCACAGTATGATCAGCTCAGTGGATGTGAAG | |
| | | TCAGAAGTTCCTGTGGGCCTGGAGCCCATCTCACCTTTAG | |
| | | ACCTAAGGACAGACCTCAGGATGATGATGCCCGTGGTGGA | |
| | | CCCTGTTGTCCGTGAGAAGCAATTGCAGCAGGAATTACTT | |
| | | CTTATCCAGCAGCAGCAACAAATCCAGAAGCAGCTTCTGA | |
| | | TAGCAGAGTTTCAGAAACAGCATGAGAACTTGACACGGCA | |
| | | GCACCAGGCTCAGCTTCAGGAGCATATCAAGGAACTTCTA | |
| | | GCCATAAAACAGCAACAAGAACTCCTAGAAAAGGAGCAGA | |
| | | AACTGGAGCAGCAGAGGCAAGAACAGGAAGTAGAGAGGCA | |
| | | TCGCAGAGAACAGCAGCTTCCTCCTCTCAGAGGCAAAGAT | |
| | | AGAGGACGAGAAAGGGCAGTGGCAAGTACAGAAGTAAAGC | |
| | | AGAAGCTTCAAGAGTTCCTACTGAGTAAATCAGCAACGAA | |
| | | AGACACTCCAACTAATGGAAAAAATCATTCCGTGAGCCGC | |
| | | CATCCCAAGCTCTGGTACACGGCTGCCCACCACACATCAT | |
| | | TGGATCAAAGCTCTCCACCCCTTAGTGGAACATCTCCATC | |
| | | CTACAAGTACACATTACCAGGAGCACAAGATGCAAAGGAT | |
| | | GATTTCCCCCTTCGAAAAACTGAATCCTCAGTCAGTAGCA | |
| | | GTTCTCCAGGCTCTGGTCCCAGTTCACCAAACAATGGGCC | |
| | | AACTGGAAGTGTTACTGAAAATGAGACTTCGGTTTTGCCC | |
| | | CCTACCCCTCATGCCGAGCAAATGGTTTCACAGCAACGCA | |
| | | TTCTAATTCATGAAGATTCCATGAACCTGCTAAGTCTTTA | |
| | | TACCTCTCCTTCTTTGCCCAACATTACCTTGGGGCTTCCC | |
| | | GCAGTGCCATCCCAGCTCAATGCTTCGAATTCACTCAAAG | |
| | | AAAAGCAGAAGTGTGAGACGCAGACGCTTAGGCAAGGTGT | |
| | | TCCTCTGCCTGGGCAGTATGGAGGCAGCATCCCGGCATCT | |
| | | TCCAGCCACCCTCATGTTACTTTAGAGGGAAAGCCACCCA | |
| | | ACAGCAGCCACCAGGCTCTCCTGCAGCATTTATTATTGAA | |
| | | AGAACAAATGCGACAGCAAAAGCTTCTTGTAGCTGGTGGA | |
| | | GTTCCCTTACATCCTCAGTCTCCCTTGGCAACAAAAGAGA | |
| | | GAATTTCACCTGGCATTAGAGGTACCCACAAATTGCCCCG | |
| | | TCACAGACCCCTGAACCGAACCCAGTCTGCACCTTTGCCT | |
| | | CAGAGCACGTTGGCTCAGCTGGTCATTCAACAGCAACACC | |
| | | AGCAATTCTTGGAGAAGCAGAAGCAATACCAGCAGCAGAT | |
| | | CCACATGAACAAACTGCTTTCGAAATCTATTGAACAACTG | |
| | | AAGCAACCAGGCAGTCACCTTGAGGAAGCAGAGGAAGAGC | |
| | | TTCAGGGGGACCAGGCGATGCAGGAAGACAGAGCGCCCTC | |
| | | TAGTGGCAACAGCACTAGGAGCGACAGCAGTGCTTGTGTG | |
| | | GATGACACACTGGGACAAGTTGGGGCTGTGAAGGTCAAGG | |
| | | AGGAACCAGTGGACAGTGATGAAGATGCTCAGATCCAGGA | |
| | | AATGGAATCTGGGGAGCAGGCTGCTTTTATGCAACAGGTA | |
| | | ATAGGCAAAGATTTAGCTCCAGGATTTGTAATTAAAGTCA | |
| | | TTATCTGAACATGAAATGCATTGCAGGTTTGGTAAATGGA | |
| | | TATGATTTCCTATCAGTTTATATTTCTCTATGATTTGAGT | |
| | | TCAGTGTTTAAGGATTCTACCTAATGCAGATATATGTATA | |
| | | TATCTATATAGAGGTCTTTCTATATACTGATCTCTATATA | |
| | | GATATCAATGTTTCATTGAAAATCCACTGGTAAGGAAATA | |
| | | CCTGTTATACTAAAATTATGATACATAATATCTGAGCAGT | |
| | | TAATAGGCTTTAAATTTATCCCAAAGCCTGCTACACCAAT | |
| | | TACTTCTAAAGAAAACAAATTCACTGTTATTTTGAGTTTA | |
| | | TGTGTTGAGATCAGTGACTGCTGGATAGTCTCCCAGTCTG | |
| | | ATCAATGAAGCATTCGATTAGTTTTTGATTTTTTGCAACA | |
| | | TCTAGAATTTAATTTTCACATCACTGTACATAATGTATCA | |
| | | TACTATAGTCTTGAACACTGTTAAAGGTAGTCTGCCCCTT | |
| | | CCTTCCTCTCTCTTTTTTTAGTTAAGTAGAAATGTTCTGG | |
| | | TCACCATGCCAGTAGTCCTAGGTTATTGTGTAGGTTGCAA | |
| | | TTGAACATATTAGGAATACAGGTGGTTTTAAATATATAGA | |
| | | TGCAAATTGCAGCACTACTTTAAATATTAGATTATGTCTC | |
| | | ACATAGCACTGCTCATTTTACTTTTATTTTGTGTAATTTG | |
| | | ATGACACTGTCTATCAAAAAAGAGCAAATGAAGCAGATGC | |
| | | AAATGTTAGTGAGAAGTAATGTGCAGCATTATGGTCCAAT | |
| | | CAGATACAATATTGTGTCTACAATTGCAAAAAACACAGTA | |
| | | ACAGGATGAATATTATCTGATATCAAGTCAAAATCAGTTT | |
| | | GAAAAGAAGGTGTATCATATTTTATATTGTCACTAGAATC | |
| | | TCTTAAGTATAATTCCATAATGACATGGGCATATACCGTA | |
| | | ACATTCTGGCAAATAACAATTAGAAAAGATAGGTTTAACA | |
| | | AAAAAATTTACTTGTATATAATGCACCTTCAGGAGGACTA | |
| | | TGTCCTTTGATGCTATAAAATACAAACAACTTTGAAGGCA | |
| | | ACAGAAGACACTGTTTATTCAAGTCAGTTCTTTGTCAGGT | |
| | | TCCTGCTGTTCTCCTACAGAAAAGTGATTCTGTGAGGGTG | |
| | | AACAGGAAATGCCTTGTGGAAACAGGAAGTCCAAGTGATT | |
| | | CATGTACTGAGGAATGTAGGAAAAAAAATCTGAGGATAGT | |
| | | GCTTTACTCTTTCTGTTTTTAAAGGGCACTCTATGAATTG | |
| | | ATTTATTGTCTAAGAAAATAACACCACAAGTAGGGAAATT | |
| | | GTTACGGAAGCTTTTCACTGGAACATTTCCTTCATATTCC | |
| | | CTTTTGATATGTTTACCTTGTTTTATAGGTTTACTTTTGT | |
| | | TAAGCTAGTTAAAGGTTCGTTGTATTAAGACCCCTTTAAT | |
| | | ATGGATAATCCAAATTGACCTAGAATCTTTGTGAGGTTTT | |
| | | TTCTATTAAAATATTTATATTTCTAAATCCGAGGTATTTC | |
| | | AAGGTGTAGTATCCTATTTCAAAGGAGATATAGCAGTTTT | |
| | | GCCAAATGTAGACATTGTTCAACTGTATGTTATTGGCACG | |
| | | TGTTGTTTACATTTTGCTGTGACATTTAAAAATATTTCTT | |
| | | TAAAAATGTTACTGCTAAAGATACATTATCCTTTTTTAAA | |
| | | AAGTCTCCATTCAAATTAAATTAACATAACTAGAAGTTAG | |
| | | AAAGTTTAAAAGTTTTCCACATAATGAAAGTCCTTCTGAT | |
| | | AATTTGACAAATAGCTATAATAGGAACACTCCCTATCACC | |
| | | AACATATTTTGGTTAGTATATTCCTTCATATTAAAATGAC | |
| | | TTTTTGTCAGTTGTTTTGCATTAAAAATATGGCATGCCTA | |
| | | AGATAAAATTGTATATTTTTTCCATCTCATAAATATTCAT | |
| | | TTTCTTCAAAGTCTTTTTTCAATCTCATAAAAAAGGGATA | |
| | | GTGCATCTTTTAAAATACATTTTATTTGGGGAGGAACATG | |
| | | TGGCTGAGCAGACTTTTGTATAATATTACTTCAAAGATAT | |
| | | GTAATCACAAACAAAAAAAACTATTTTTTATAATGTCATT | |
| | | TGAGAGAGTTTCATCAGTACAGTTGGTGGACGTTAATTGT | |
| | | TTGAATTTGATAGTCTTTGAATTTAATCAAGAAACTACCT | |
| | | GGAACCAGTGAAAAGGAAAGCTGGACTTAAATAATCTTAG | |
| | | AATTAATTGATAAATGTCTCTTTTAAAATCTACTGTATTT | |
| | | ATTATAATTTACACCCTTGAAGGTGATCTCTTGTTTTGTG | |
| | | TTGTAAATATATTGTTTGTATGTTTCCCTTCTTGCCTTCT | |
| | | GTTATAAGTCTCTTCCTTTCTCAAATAAAGTTTTTTTTAA | |
| | | AAGAAAAAAAAAAAAAAAAAAAA | |
| HSF2 | NM_004506.3 | ACTTGTCCGTCACGTGCGGCCGCCCGGCCTCTCGGCCTTG | 18 |
| | | CCGCGCGCCTGGCGGGGTTGGGGGGGCGGGGACCAAGATC | |
| | | TGCTGCGCCTGCGTTGTGGGCGTTCTCGGGGAGCTGCTGC | |
| | | CGTAGCTGCCGCCGCCGCTACCACCGCGTTCGGGTGTAGA | |
| | | ATTTGGAATCCCTGCGCCGCGTTAACAATGAAGCAGAGTT | |
| | | CGAACGTGCCGGCTTTCCTCAGCAAGCTGTGGACGCTTGT | |
| | | GGAGGAAACCCACACTAACGAGTTCATCACCTGGAGCCAG | |
| | | AATGGCCAAAGTTTTCTGGTCTTGGATGAGCAACGATTTG | |
| | | CAAAAGAAATTCTTCCCAAATATTTCAAGCACAATAATAT | |
| | | GGCAAGCTTTGTGAGGCAACTGAATATGTATGGTTTCCGT | |
| | | AAAGTAGTACATATCGACTCTGGAATTGTAAAGCAAGAAA | |
| | | GAGATGGTCCTGTAGAATTTCAGCATCCTTACTTCAAACA | |
| | | AGGACAGGATGACTTGTTGGAGAACATTAAAAGGAAGGTT | |
| | | TCATCTTCAAAACCAGAAGAAAATAAAATTCGTCAGGAAG | |
| | | ATTTAACAAAAATTATAAGTAGTGCTCAGAAGGTTCAGAT | |
| | | AAAACAGGAAACTATTGAGTCCAGGCTTTCTGAATTAAAA | |
| | | AGTGAGAATGAGTCCCTTTGGAAGGAGGTGTCAGAATTAC | |
| | | GAGCAAAGCATGCACAACAGCAACAAGTTATTCGAAAGAT | |
| | | TGTCCAGTTTATTGTTACATTGGTTCAAAATAACCAACTT | |
| | | GTGAGTTTAAAACGTAAAAGGCCTCTACTTCTAAACACTA | |
| | | ATGGAGCCCAAAAGAAGAACCTGTTTCAGCACATAGTCAA | |
| | | AGAACCAACTGATAATCATCATCATAAAGTTCCACACAGT | |
| | | AGGACTGAAGGTTTAAAGCCAAGGGAGAGGATTTCAGATG | |
| | | ACATCATTATTTATGATGTTACTGATGATAATGCAGATGA | |
| | | AGAAAATATCCCAGTTATTCCAGAAACTAATGAGGATGTT | |
| | | ATATCTGATCCCTCCAACTGTAGCCAGTACCCTGATATTG | |
| | | TCATCGTTGAAGATGACAATGAAGATGAGTATGCACCTGT | |
| | | CATTCAGAGTGGAGAGCAGAATGAACCAGCCAGAGAATCC | |
| | | CTAAGTTCAGGCAGTGATGGCAGCAGCCCTCTCATGTCTA | |
| | | GTGCTGTCCAGCTAAATGGCTCATCCAGTCTGACCTCAGA | |
| | | AGATCCAGTGACCATGATGGATTCCATTTTGAATGATAAC | |
| | | ATCAATCTTTTGGGAAAGGTTGAGCTGTTGGATTATCTTG | |
| | | ACAGTATTGACTGCAGTTTAGAGGACTTCCAGGCCATGCT | |
| | | ATCAGGAAGACAATTTAGCATAGACCCAGATCTCCTGGTT | |
| | | GATCTTTTCACTAGTTCTGTGCAGATGAATCCCACAGATT | |
| | | ACATCAATAATACAAAATCTGAGAATAAAGGATTAGAAAC | |
| | | TACCAAGAACAATGTAGTTCAGCCAGTTTCGGAAGAGGGA | |
| | | AGAAAATCTAAATCCAAACCAGATAAGCAGCTTATCCAGT | |
| | | ATACCGCCTTTCCACTTCTTGCATTCCTCGATGGGAACCC | |
| | | TGCTTCTTCTGTTGAACAGGCGAGTACAACAGCATCATCA | |
| | | GAAGTTTTGTCCTCTGTAGATAAACCCATAGAAGTTGATG | |
| | | AGCTTCTGGATAGCAGCCTAGACCCAGAACCAACCCAAAG | |
| | | TAAGCTTGTTCGCCTGGAGCCATTGACTGAAGCTGAAGCT | |
| | | AGTGAAGCTACACTGTTTTATTTATGTGAACTTGCTCCTG | |
| | | CACCTCTGGATAGTGATATGCCACTTTTAGATAGCTAAAT | |
| | | CCCCAGGAAGTGGACTTTACATGTATATATTCATCAAAAT | |
| | | GATGAACTATTTATTTTAAAGTATCATTTGGTACTTTTTT | |
| | | TGTAAATTGCTTTGTTTTGTTTAATCAGATACTGTGGAAT | |
| | | AAAAGCACCTTTTGCTTTTCTCACTAACCACACACTCTTG | |
| | | CAGAGCTTTCAGGTGTTACTCAGCTGCATAGTTACGCAGA | |
| | | TGTAATGCACATTATTGGCGTATCTTTAAGTTGGATTCAA | |
| | | ATGGCCATTTTTCTCCAATTTTGGTAAATTGGATATCTTT | |
| | | TTTTTACAAATACGACCATTAACCTCAGTTAAATTTTTGT | |
| | | TTGTTTTCCTGTTTGATGCTGTCTATTTGCATTGAGTGTA | |
| | | AGTCATTTGAACTAATGGTATAACTCCTAAAGCTTTCTCT | |
| | | GCTCCAGTTATTTTTATTAAATATTTTTCACTTGGCTTAT | |
| | | TTTTAAAACTGGGAACATAAAGTGCCTGTATCTTGTAAAA | |
| | | CTTCATTTGTTTCTTTTGGTTCAGAGAAGTTCATTTATGT | |
| | | TCAAAGACGTTTATTCATGTTCAACAGGAAAGACAAAGTG | |
| | | TACGTGAATGCTCGCTGTCTGATAGGGTTCCAGCTCCATA | |
| | | TATATAGAAAGATCGGGGGTGGGATGGGATGGAGTGAGCC | |
| | | CCATCCAGTTAGTTGGACTAGTTTTAAATAAAGGTTTTCC | |
| | | GGTTTGTGTTTTTTTGAACCATACTGTTTAGTAAAATAAA | |
| | | TACAATGAATGTTGAGTACTAGTGTCTGTTATGTGTCTTC | |
| | | TTTAGAGGTGACACTCACATGAAACAATTTTTTCTTCTCA | |
| | | TAGGAAGCAGTAGCTTTAAACTGTCTGTGGTTCATTATTC | |
| | | TCAATATGAATCATACCAAGATATTTGTGCCTCATCTCGA | |
| | | AAATATATTGTATATTG | |
| Ki-67 | NM_001145966.1 | TACCGGGCGGAGGTGAGCGCGGCGCCGGCTCCTCCTGCGG | 19 |
| | | CGGACTTTGGGTGCGACTTGACGAGCGGTGGTTCGACAAG | |
| | | TGGCCTTGCGGGCCGGATCGTCCCAGTGGAAGAGTTGTAA | |
| | | ATTTGCTTCTGGCCTTCCCCTACGGATTATACCTGGCCTT | |
| | | CCCCTACGGATTATACTCAACTTACTGTTTAGAAAATGTG | |
| | | GCCCACGAGACGCCTGGTTACTATCAAAAGGAGCGGGGTC | |
| | | GACGGTCCCCACTTTCCCCTGAGCCTCAGCACCTGCTTGT | |
| | | TTGGAAGGGGTATTGAATGTGACATCCGTATCCAGCTTCC | |
| | | TGTTGTGTCAAAACAACATTGCAAAATTGAAATCCATGAG | |
| | | CAGGAGGCAATATTACATAATTTCAGTTCCACAAATCCAA | |
| | | CACAAGTAAATGGGTCTGTTATTGATGAGCCTGTACGGCT | |
| | | AAAACATGGAGATGTAATAACTATTATTGATCGTTCCTTC | |
| | | AGGTATGAAAATGAAAGTCTTCAGAATGGAAGGAAGTCAA | |
| | | CTGAATTTCCAAGAAAAATACGTGAACAGGAGCCAGCACG | |
| | | TCGTGTCTCAAGATCTAGCTTCTCTTCTGACCCTGATGAG | |
| | | AGTGAGGGAATACCTTTGAAAAGAAGGCGTGTGTCCTTTG | |
| | | GTGGGCACCTAAGACCTGAACTATTTGATGAAAACTTGCC | |
| | | TCCTAATACGCCTCTCAAAAGGGGAGAAGCCCCAACCAAA | |
| | | AGAAAGTCTCTGGTAATGCACACTCCACCTGTCCTGAAGA | |
| | | AAATCATCAAGGAACAGCCTCAACCATCAGGAAAACAAGA | |
| | | GTCAGGTTCAGAAATCCATGTGGAAGTGAAGGCACAAAGC | |
| | | TTGGTTATAAGCCCTCCAGCTCCTAGTCCTAGGAAAACTC | |
| | | CAGTTGCCAGTGATCAACGCCGTAGGTCCTGCAAAACAGC | |
| | | CCCTGCTTCCAGCAGCAAATCTCAGACAGAGGTTCCTAAG | |
| | | AGAGGAGGGAGAAAGAGTGGCAACCTGCCTTCAAAGAGAG | |
| | | TGTCTATCAGCCGAAGTCAACATGATATTTTACAGATGAT | |
| | | ATGTTCCAAAAGAAGAAGTGGTGCTTCGGAAGCAAATCTG | |
| | | ATTGTTGCAAAATCATGGGCAGATGTAGTAAAACTTGGTG | |
| | | CAAAACAAACACAAACTAAAGTCATAAAACATGGTCCTCA | |
| | | AAGGTCAATGAACAAAAGGCAAAGAAGACCTGCTACTCCA | |
| | | AAGAAGCCTGTGGGCGAAGTTCACAGTCAATTTAGTACAG | |
| | | GCCACGCAAACTCTCCTTGTACCATAATAATAGGGAAAGC | |
| | | TCATACTGAAAAAGTACATGTGCCTGCTCGACCCTACAGA | |
| | | GTGCTCAACAACTTCATTTCCAACCAAAAAATGGACTTTA | |
| | | AGGAAGATCTTTCAGGAATAGCTGAAATGTTCAAGACCCC | |
| | | AGTGAAGGAGCAACCGCAGTTGACAAGCACATGTCACATC | |
| | | GCTATTTCAAATTCAGAGAATTTGCTTGGAAAACAGTTTC | |
| | | AAGGAACTGATTCAGGAGAAGAACCTCTGCTCCCCACCTC | |
| | | AGAGAGTTTTGGAGGAAATGTGTTCTTCAGTGCACAGAAT | |
| | | GCAGCAAAACAGCCATCTGATAAATGCTCTGCAAGCCCTC | |
| | | CCTTAAGACGGCAGTGTATTAGAGAAAATGGAAACGTAGC | |
| | | AAAAACGCCCAGGAACACCTACAAAATGACTTCTCTGGAG | |
| | | ACAAAAACTTCAGATACTGAGACAGAGCCTTCAAAAACAG | |
| | | TATCCACTGCAAACAGGTCAGGAAGGTCTACAGAGTTCAG | |
| | | GAATATACAGAAGCTACCTGTGGAAAGTAAGAGTGAAGAA | |
| | | ACAAATACAGAAATTGTTGAGTGCATCCTAAAAAGAGGTC | |
| | | AGAAGGCAACACTACTACAACAAAGGAGAGAAGGAGAGAT | |
| | | GAAGGAAATAGAAAGACCTTTTGAGACATATAAGGAAAAT | |
| | | ATTGAATTAAAAGAAAACGATGAAAAGATGAAAGCAATGA | |
| | | AGAGATCAAGAACTTGGGGGCAGAAATGTGCACCAATGTC | |
| | | TGACCTGACAGACCTCAAGAGCTTGCCTGATACAGAACTC | |
| | | ATGAAAGACACGGCACGTGGCCAGAATCTCCTCCAAACCC | |
| | | AAGATCATGCCAAGGCACCAAAGAGTGAGAAAGGCAAAAT | |
| | | CACTAAAATGCCCTGCCAGTCATTACAACCAGAACCAATA | |
| | | AACACCCCAACACACACAAAACAACAGTTGAAGGCATCCC | |
| | | TGGGGAAAGTAGGTGTGAAAGAAGAGCTCCTAGCAGTCGG | |
| | | CAAGTTCACACGGACGTCAGGGGAGACCACGCACACGCAC | |
| | | AGAGAGCCAGCAGGAGATGGCAAGAGCATCAGAACGTTTA | |
| | | AGGAGTCTCCAAAGCAGATCCTGGACCCAGCAGCCCGTGT | |
| | | AACTGGAATGAAGAAGTGGCCAAGAACGCCTAAGGAAGAG | |
| | | GCCCAGTCACTAGAAGACCTGGCTGGCTTCAAAGAGCTCT | |
| | | TCCAGACACCAGGTCCCTCTGAGGAATCAATGACTGATGA | |
| | | GAAAACTACCAAAATAGCCTGCAAATCTCCACCACCAGAA | |
| | | TCAGTGGACACTCCAACAAGCACAAAGCAATGGCCTAAGA | |
| | | GAAGTCTCAGGAAAGCAGATGTAGAGGAAGAATTCTTAGC | |
| | | ACTCAGGAAACTAACACCATCAGCAGGGAAAGCCATGCTT | |
| | | ACGCCCAAACCAGCAGGAGGTGATGAGAAAGACATTAAAG | |
| | | CATTTATGGGAACTCCAGTGCAGAAACTGGACCTGGCAGG | |
| | | AACTTTACCTGGCAGCAAAAGACAGCTACAGACTCCTAAG | |
| | | GAAAAGGCCCAGGCTCTAGAAGACCTGGCTGGCTTTAAAG | |
| | | AGCTCTTCCAGACTCCTGGTCACACCGAGGAATTAGTGGC | |
| | | TGCTGGTAAAACCACTAAAATACCCTGCGACTCTCCACAG | |
| | | TCAGACCCAGTGGACACCCCAACAAGCACAAAGCAACGAC | |
| | | CCAAGAGAAGTATCAGGAAAGCAGATGTAGAGGGAGAACT | |
| | | CTTAGCGTGCAGGAATCTAATGCCATCAGCAGGCAAAGCC | |
| | | ATGCACACGCCTAAACCATCAGTAGGTGAAGAGAAAGACA | |
| | | TCATCATATTTGTGGGAACTCCAGTGCAGAAACTGGACCT | |
| | | GACAGAGAACTTAACCGGCAGCAAGAGACGGCCACAAACT | |
| | | CCTAAGGAAGAGGCCCAGGCTCTGGAAGACCTGACTGGCT | |
| | | TTAAAGAGCTCTTCCAGACCCCTGGTCATACTGAAGAAGC | |
| | | AGTGGCTGCTGGCAAAACTACTAAAATGCCCTGCGAATCT | |
| | | TCTCCACCAGAATCAGCAGACACCCCAACAAGCACAAGAA | |
| | | GGCAGCCCAAGACACCTTTGGAGAAAAGGGACGTACAGAA | |
| | | GGAGCTCTCAGCCCTGAAGAAGCTCACACAGACATCAGGG | |
| | | GAAACCACACACACAGATAAAGTACCAGGAGGTGAGGATA | |
| | | AAAGCATCAACGCGTTTAGGGAAACTGCAAAACAGAAACT | |
| | | GGACCCAGCAGCAAGTGTAACTGGTAGCAAGAGGCACCCA | |
| | | AAAACTAAGGAAAAGGCCCAACCCCTAGAAGACCTGGCTG | |
| | | GCTTGAAAGAGCTCTTCCAGACACCAGTATGCACTGACAA | |
| | | GCCCACGACTCACGAGAAAACTACCAAAATAGCCTGCAGA | |
| | | TCACAACCAGACCCAGTGGACACACCAACAAGCTCCAAGC | |
| | | CACAGTCCAAGAGAAGTCTCAGGAAAGTGGACGTAGAAGA | |
| | | AGAATTCTTCGCACTCAGGAAACGAACACCATCAGCAGGC | |
| | | AAAGCCATGCACACACCCAAACCAGCAGTAAGTGGTGAGA | |
| | | AAAACATCTACGCATTTATGGGAACTCCAGTGCAGAAACT | |
| | | GGACCTGACAGAGAACTTAACTGGCAGCAAGAGACGGCTA | |
| | | CAAACTCCTAAGGAAAAGGCCCAGGCTCTAGAAGACCTGG | |
| | | CTGGCTTTAAAGAGCTCTTCCAGACACGAGGTCACACTGA | |
| | | GGAATCAATGACTAACGATAAAACTGCCAAAGTAGCCTGC | |
| | | AAATCTTCACAACCAGACCCAGACAAAAACCCAGCAAGCT | |
| | | CCAAGCGACGGCTCAAGACATCCCTGGGGAAAGTGGGCGT | |
| | | GAAAGAAGAGCTCCTAGCAGTTGGCAAGCTCACACAGACA | |
| | | TCAGGAGAGACTACACACACACACACAGAGCCAACAGGAG | |
| | | ATGGTAAGAGCATGAAAGCATTTATGGAGTCTCCAAAGCA | |
| | | GATCTTAGACTCAGCAGCAAGTCTAACTGGCAGCAAGAGG | |
| | | CAGCTGAGAACTCCTAAGGGAAAGTCTGAAGTCCCTGAAG | |
| | | ACCTGGCCGGCTTCATCGAGCTCTTCCAGACACCAAGTCA | |
| | | CACTAAGGAATCAATGACTAACGAAAAAACTACCAAAGTA | |
| | | TCCTACAGAGCTTCACAGCCAGACCTAGTGGACACCCCAA | |
| | | CAAGCTCCAAGCCACAGCCCAAGAGAAGTCTCAGGAAAGC | |
| | | AGACACTGAAGAAGAATTTTTAGCATTTAGGAAACAAACG | |
| | | CCATCAGCAGGCAAAGCCATGCACACACCCAAACCAGCAG | |
| | | TAGGTGAAGAGAAAGACATCAACACGTTTTTGGGAACTCC | |
| | | AGTGCAGAAACTGGACCAGCCAGGAAATTTACCTGGCAGC | |
| | | AATAGACGGCTACAAACTCGTAAGGAAAAGGCCCAGGCTC | |
| | | TAGAAGAACTGACTGGCTTCAGAGAGCTTTTCCAGACACC | |
| | | ATGCACTGATAACCCCACGACTGATGAGAAAACTACCAAA | |
| | | AAAATACTCTGCAAATCTCCGCAATCAGACCCAGCGGACA | |
| | | CCCCAACAAACACAAAGCAACGGCCCAAGAGAAGCCTCAA | |
| | | GAAAGCAGACGTAGAGGAAGAATTTTTAGCATTCAGGAAA | |
| | | CTAACACCATCAGCAGGCAAAGCCATGCACACGCCTAAAG | |
| | | CAGCAGTAGGTGAAGAGAAAGACATCAACACATTTGTGGG | |
| | | GACTCCAGTGGAGAAACTGGACCTGCTAGGAAATTTACCT | |
| | | GGCAGCAAGAGACGGCCACAAACTCCTAAAGAAAAGGCCA | |
| | | AGGCTCTAGAAGATCTGGCTGGCTTCAAAGAGCTCTTCCA | |
| | | GACACCAGGTCACACTGAGGAATCAATGACCGATGACAAA | |
| | | ATCACAGAAGTATCCTGCAAATCTCCACAACCAGACCCAG | |
| | | TCAAAACCCCAACAAGCTCCAAGCAACGACTCAAGATATC | |
| | | CTTGGGGAAAGTAGGTGTGAAAGAAGAGGTCCTACCAGTC | |
| | | GGCAAGCTCACACAGACGTCAGGGAAGACCACACAGACAC | |
| | | ACAGAGAGACAGCAGGAGATGGAAAGAGCATCAAAGCGTT | |
| | | TAAGGAATCTGCAAAGCAGATGCTGGACCCAGCAAACTAT | |
| | | GGAACTGGGATGGAGAGGTGGCCAAGAACACCTAAGGAAG | |
| | | AGGCCCAATCACTAGAAGACCTGGCCGGCTTCAAAGAGCT | |
| | | CTTCCAGACACCAGACCACACTGAGGAATCAACAACTGAT | |
| | | GACAAAACTACCAAAATAGCCTGCAAATCTCCACCACCAG | |
| | | AATCAATGGACACTCCAACAAGCACAAGGAGGCGGCCCAA | |
| | | AACACCTTTGGGGAAAAGGGATATAGTGGAAGAGCTCTCA | |
| | | GCCCTGAAGCAGCTCACACAGACCACACACACAGACAAAG | |
| | | TACCAGGAGATGAGGATAAAGGCATCAACGTGTTCAGGGA | |
| | | AACTGCAAAACAGAAACTGGACCCAGCAGCAAGTGTAACT | |
| | | GGTAGCAAGAGGCAGCCAAGAACTCCTAAGGGAAAAGCCC | |
| | | AACCCCTAGAAGACTTGGCTGGCTTGAAAGAGCTCTTCCA | |
| | | GACACCAATATGCACTGACAAGCCCACGACTCATGAGAAA | |
| | | ACTACCAAAATAGCCTGCAGATCTCCACAACCAGACCCAG | |
| | | TGGGTACCCCAACAATCTTCAAGCCACAGTCCAAGAGAAG | |
| | | TCTCAGGAAAGCAGACGTAGAGGAAGAATCCTTAGCACTC | |
| | | AGGAAACGAACACCATCAGTAGGGAAAGCTATGGACACAC | |
| | | CCAAACCAGCAGGAGGTGATGAGAAAGACATGAAAGCATT | |
| | | TATGGGAACTCCAGTGCAGAAATTGGACCTGCCAGGAAAT | |
| | | TTACCTGGCAGCAAAAGATGGCCACAAACTCCTAAGGAAA | |
| | | AGGCCCAGGCTCTAGAAGACCTGGCTGGCTTCAAAGAGCT | |
| | | CTTCCAGACACCAGGCACTGACAAGCCCACGACTGATGAG | |
| | | AAAACTACCAAAATAGCCTGCAAATCTCCACAACCAGACC | |
| | | CAGTGGACACCCCAGCAAGCACAAAGCAACGGCCCAAGAG | |
| | | AAACCTCAGGAAAGCAGACGTAGAGGAAGAATTTTTAGCA | |
| | | CTCAGGAAACGAACACCATCAGCAGGCAAAGCCATGGACA | |
| | | CACCAAAACCAGCAGTAAGTGATGAGAAAAATATCAACAC | |
| | | ATTTGTGGAAACTCCAGTGCAGAAACTGGACCTGCTAGGA | |
| | | AATTTACCTGGCAGCAAGAGACAGCCACAGACTCCTAAGG | |
| | | AAAAGGCTGAGGCTCTAGAGGACCTGGTTGGCTTCAAAGA | |
| | | ACTCTTCCAGACACCAGGTCACACTGAGGAATCAATGACT | |
| | | GATGACAAAATCACAGAAGTATCCTGTAAATCTCCACAGC | |
| | | CAGAGTCATTCAAAACCTCAAGAAGCTCCAAGCAAAGGCT | |
| | | CAAGATACCCCTGGTGAAAGTGGACATGAAAGAAGAGCCC | |
| | | CTAGCAGTCAGCAAGCTCACACGGACATCAGGGGAGACTA | |
| | | CGCAAACACACACAGAGCCAACAGGAGATAGTAAGAGCAT | |
| | | CAAAGCGTTTAAGGAGTCTCCAAAGCAGATCCTGGACCCA | |
| | | GCAGCAAGTGTAACTGGTAGCAGGAGGCAGCTGAGAACTC | |
| | | GTAAGGAAAAGGCCCGTGCTCTAGAAGACCTGGTTGACTT | |
| | | CAAAGAGCTCTTCTCAGCACCAGGTCACACTGAAGAGTCA | |
| | | ATGACTATTGACAAAAACACAAAAATTCCCTGCAAATCTC | |
| | | CCCCACCAGAACTAACAGACACTGCCACGAGCACAAAGAG | |
| | | ATGCCCCAAGACACGTCCCAGGAAAGAAGTAAAAGAGGAG | |
| | | CTCTCAGCAGTTGAGAGGCTCACGCAAACATCAGGGCAAA | |
| | | GCACACACACACACAAAGAACCAGCAAGCGGTGATGAGGG | |
| | | CATCAAAGTATTGAAGCAACGTGCAAAGAAGAAACCAAAC | |
| | | CCAGTAGAAGAGGAACCCAGCAGGAGAAGGCCAAGAGCAC | |
| | | CTAAGGAAAAGGCCCAACCCCTGGAAGACCTGGCCGGCTT | |
| | | CACAGAGCTCTCTGAAACATCAGGTCACACTCAGGAATCA | |
| | | CTGACTGCTGGCAAAGCCACTAAAATACCCTGCGAATCTC | |
| | | CCCCACTAGAAGTGGTAGACACCACAGCAAGCACAAAGAG | |
| | | GCATCTCAGGACACGTGTGCAGAAGGTACAAGTAAAAGAA | |
| | | GAGCCTTCAGCAGTCAAGTTCACACAAACATCAGGGGAAA | |
| | | CCACGGATGCAGACAAAGAACCAGCAGGTGAAGATAAAGG | |
| | | CATCAAAGCATTGAAGGAATCTGCAAAACAGACACCGGCT | |
| | | CCAGCAGCAAGTGTAACTGGCAGCAGGAGACGGCCAAGAG | |
| | | CACCCAGGGAAAGTGCCCAAGCCATAGAAGACCTAGCTGG | |
| | | CTTCAAAGACCCAGCAGCAGGTCACACTGAAGAATCAATG | |
| | | ACTGATGACAAAACCACTAAAATACCCTGCAAATCATCAC | |
| | | CAGAACTAGAAGACACCGCAACAAGCTCAAAGAGACGGCC | |
| | | CAGGACACGTGCCCAGAAAGTAGAAGTGAAGGAGGAGCTG | |
| | | TTAGCAGTTGGCAAGCTCACACAAACCTCAGGGGAGACCA | |
| | | CGCACACCGACAAAGAGCCGGTAGGTGAGGGCAAAGGCAC | |
| | | GAAAGCATTTAAGCAACCTGCAAAGCGGAAGCTGGACGCA | |
| | | GAAGATGTAATTGGCAGCAGGAGACAGCCAAGAGCACCTA | |
| | | AGGAAAAGGCCCAACCCCTGGAAGATCTGGCCAGCTTCCA | |
| | | AGAGCTCTCTCAAACACCAGGCCACACTGAGGAACTGGCA | |
| | | AATGGTGCTGCTGATAGCTTTACAAGCGCTCCAAAGCAAA | |
| | | CACCTGACAGTGGAAAACCTCTAAAAATATCCAGAAGAGT | |
| | | TCTTCGGGCCCCTAAAGTAGAACCCGTGGGAGACGTGGTA | |
| | | AGCACCAGAGACCCTGTAAAATCACAAAGCAAAAGCAACA | |
| | | CTTCCCTGCCCCCACTGCCCTTCAAGAGGGGAGGTGGCAA | |
| | | AGATGGAAGCGTCACGGGAACCAAGAGGCTGCGCTGCATG | |
| | | CCAGCACCAGAGGAAATTGTGGAGGAGCTGCCAGCCAGCA | |
| | | AGAAGCAGAGGGTTGCTCCCAGGGCAAGAGGCAAATCATC | |
| | | CGAACCCGTGGTCATCATGAAGAGAAGTTTGAGGACTTCT | |
| | | GCAAAAAGAATTGAACCTGCGGAAGAGCTGAACAGCAACG | |
| | | ACATGAAAACCAACAAAGAGGAACACAAATTACAAGACTC | |
| | | GGTCCCTGAAAATAAGGGAATATCCCTGCGCTCCAGACGC | |
| | | CAAAATAAGACTGAGGCAGAACAGCAAATAACTGAGGTCT | |
| | | TTGTATTAGCAGAAAGAATAGAAATAAACAGAAATGAAAA | |
| | | GAAGCCCATGAAGACCTCCCCAGAGATGGACATTCAGAAT | |
| | | CCAGATGATGGAGCCCGGAAACCCATACCTAGAGACAAAG | |
| | | TCACTGAGAACAAAAGGTGCTTGAGGTCTGCTAGACAGAA | |
| | | TGAGAGCTCCCAGCCTAAGGTGGCAGAGGAGAGCGGAGGG | |
| | | CAGAAGAGTGCGAAGGTTCTCATGCAGAATCAGAAAGGGA | |
| | | AAGGAGAAGCAGGAAATTCAGACTCCATGTGCCTGAGATC | |
| | | AAGAAAGACAAAAAGCCAGCCTGCAGCAAGCACTTTGGAG | |
| | | AGCAAATCTGTGCAGAGAGTAACGCGGAGTGTCAAGAGGT | |
| | | GTGCAGAAAATCCAAAGAAGGCTGAGGACAATGTGTGTGT | |
| | | CAAGAAAATAAGAACCAGAAGTCATAGGGACAGTGAAGAT | |
| | | ATTTGACAGAAAAATCGAACTGGGAAAAATATAATAAAGT | |
| | | TAGTTTTGTGATAAGTTCTAGTGCAGTTTTTGTCATAAAT | |
| | | TACAAGTGAATTCTGTAAGTAAGGCTGTCAGTCTGCTTAA | |
| | | GGGAAGAAAACTTTGGATTTGCTGGGTCTGAATCGGCTTC | |
| | | ATAAACTCCACTGGGAGCACTGCTGGGCTCCTGGACTGAG | |
| | | AATAGTTGAACACCGGGGGCTTTGTGAAGGAGTCTGGGCC | |
| | | AAGGTTTGCCCTCAGCTTTGCAGAATGAAGCCTTGAGGTC | |
| | | TGTCACCACCCACAGCCACCCTACAGCAGCCTTAACTGTG | |
| | | ACACTTGCCACACTGTGTCGTCGTTTGTTTGCCTATGTCC | |
| | | TCCAGGGCACGGTGGCAGGAACAACTATCCTCGTCTGTCC | |
| | | CAACACTGAGCAGGCACTCGGTAAACACGAATGAATGGAT | |
| | | GAGCGCACGGATGAATGGAGCTTACAAGATCTGTCTTTCC | |
| | | AATGGCCGGGGGCATTTGGTCCCCAAATTAAGGCTATTGG | |
| | | ACATCTGCACAGGACAGTCCTATTTTTGATGTCCTTTCCT | |
| | | TTCTGAAAATAAAGTTTTGTGCTTTGGAGAATGACTCGTG | |
| | | AGCACATCTTTAGGGACCAAGAGTGACTTTCTGTAAGGAG | |
| | | TGACTCGTGGCTTGCCTTGGTCTCTTGGGAATACTTTTCT | |
| | | AACTAGGGTTGCTCTCACCTGAGACATTCTCCACCCGCGG | |
| | | AATCTCAGGGTCCCAGGCTGTGGGCCATCACGACCTCAAA | |
| | | CTGGCTCCTAATCTCCAGCTTTCCTGTCATTGAAAGCTTC | |
| | | GGAAGTTTACTGGCTCTGCTCCCGCCTGTTTTCTTTCTGA | |
| | | CTCTATCTGGCAGCCCGATGCCACCCAGTACAGGAAGTGA | |
| | | CACCAGTACTCTGTAAAGCATCATCATCCTTGGAGAGACT | |
| | | GAGCACTCAGCACCTTCAGCCACGATTTCAGGATCGCTTC | |
| | | CTTGTGAGCCGCTGCCTCCGAAATCTCCTTTGAAGCCCAG | |
| | | ACATCTTTCTCCAGCTTCAGACTTGTAGATATAACTCGTT | |
| | | CATCTTCATTTACTTTCCACTTTGCCCCCTGTCCTCTCTG | |
| | | TGTTCCCCAAATCAGAGAATAGCCCGCCATCCCCCAGGTC | |
| | | ACCTGTCTGGATTCCTCCCCATTCACCCACCTTGCCAGGT | |
| | | GCAGGTGAGGATGGTGCACCAGACAGGGTAGCTGTCCCCC | |
| | | AAAATGTGCCCTGTGCGGGCAGTGCCCTGTCTCCACGTTT | |
| | | GTTTCCCCAGTGTCTGGCGGGGAGCCAGGTGACATCATAA | |
| | | ATACTTGCTGAATGAATGCAGAAATCAGCGGTACTGACTT | |
| | | GTACTATATTGGCTGCCATGATAGGGTTCTCACAGCGTCA | |
| | | TCCATGATCGTAAGGGAGAATGACATTCTGCTTGAGGGAG | |
| | | GGAATAGAAAGGGGCAGGGAGGGGACATCTGAGGGCTTCA | |
| | | CAGGGCTGCAAAGGGTACAGGGATTGCACCAGGGCAGAAC | |
| | | AGGGGAGGGTGTTCAAGGAAGAGTGGCTCTTAGCAGAGGC | |
| | | ACTTTGGAAGGTGTGAGGCATAAATGCTTCCTTCTACGTA | |
| | | GGCCAACCTCAAAACTTTCAGTAGGAATGTTGCTATGATC | |
| | | AAGTTGTTCTAACACTTTAGACTTAGTAGTAATTATGAAC | |
| | | CTCACATAGAAAAATTTCATCCAGCCATATGCCTGTGGAG | |
| | | TGGAATATTCTGTTTAGTAGAAAAATCCTTTAGAGTTCAG | |
| | | CTCTAACCAGAAATCTTGCTGAAGTATGTCAGCACCTTTT | |
| | | CTCACCCTGGTAAGTACAGTATTTCAAGAGCACGCTAAGG | |
| | | GTGGTTTTCATTTTACAGGGCTGTTGATGATGGGTTAAAA | |
| | | ATGTTCATTTAAGGGCTACCCCCGTGTTTAATAGATGAAC | |
| | | ACCACTTCTACACAACCCTCCTTGGTACTGGGGGAGGGAG | |
| | | AGATCTGACAAATACTGCCCATTCCCCTAGGCTGACTGGA | |
| | | TTTGAGAACAAATACCCACCCATTTCCACCATGGTATGGT | |
| | | AACTTCTCTGAGCTTCAGTTTCCAAGTGAATTTCCATGTA | |
| | | ATAGGACATTCCCATTAAATACAAGCTGTTTTTACTTTTT | |
| | | CGCCTCCCAGGGCCTGTGGGATCTGGTCCCCCAGCCTCTC | |
| | | TTGGGCTTTCTTACACTAACTCTGTACCTACCATCTCCTG | |
| | | CCTCCCTTAGGCAGGCACCTCCAACCACCACACACTCCCT | |
| | | GCTGTTTTCCCTGCCTGGAACTTTCCCTCCTGCCCCACCA | |
| | | AGATCATTTCATCCAGTCCTGAGCTCAGCTTAAGGGAGGC | |
| | | TTCTTGCCTGTGGGTTCCCTCACCCCCATGCCTGTCCTCC | |
| | | AGGCTGGGGCAGGTTCTTAGTTTGCCTGGAATTGTTCTGT | |
| | | ACCTCTTTGTAGCACGTAGTGTTGTGGAAACTAAGCCACT | |
| | | AATTGAGTTTCTGGCTCCCCTCCTGGGGTTGTAAGTTTTG | |
| | | TTCATTCATGAGGGCCGACTGCATTTCCTGGTTACTCTAT | |
| | | CCCAGTGACCAGCCACAGGAGATGTCCAATAAAGTATGTG | |
| | | ATGAAATGGTCTTAAAAAAAAAAAAAA | |
| KRAS | NM_004985.4 | TCCTAGGCGGCGGCCGCGGCGGCGGAGGCAGCAGCGGCGG | 20 |
| | | CGGCAGTGGCGGCGGCGAAGGTGGCGGCGGCTCGGCCAGT | |
| | | ACTCCCGGCCCCCGCCATTTCGGACTGGGAGCGAGCGCGG | |
| | | CGCAGGCACTGAAGGCGGCGGCGGGGCCAGAGGCTCAGCG | |
| | | GCTCCCAGGTGCGGGAGAGAGGCCTGCTGAAAATGACTGA | |
| | | ATATAAACTTGTGGTAGTTGGAGCTGGTGGCGTAGGCAAG | |
| | | AGTGCCTTGACGATACAGCTAATTCAGAATCATTTTGTGG | |
| | | ACGAATATGATCCAACAATAGAGGATTCCTACAGGAAGCA | |
| | | AGTAGTAATTGATGGAGAAACCTGTCTCTTGGATATTCTC | |
| | | GACACAGCAGGTCAAGAGGAGTACAGTGCAATGAGGGACC | |
| | | AGTACATGAGGACTGGGGAGGGCTTTCTTTGTGTATTTGC | |
| | | CATAAATAATACTAAATCATTTGAAGATATTCACCATTAT | |
| | | AGAGAACAAATTAAAAGAGTTAAGGACTCTGAAGATGTAC | |
| | | CTATGGTCCTAGTAGGAAATAAATGTGATTTGCCTTCTAG | |
| | | AACAGTAGACACAAAACAGGCTCAGGACTTAGCAAGAAGT | |
| | | TATGGAATTCCTTTTATTGAAACATCAGCAAAGACAAGAC | |
| | | AGGGTGTTGATGATGCCTTCTATACATTAGTTCGAGAAAT | |
| | | TCGAAAACATAAAGAAAAGATGAGCAAAGATGGTAAAAAG | |
| | | AAGAAAAAGAAGTCAAAGACAAAGTGTGTAATTATGTAAA | |
| | | TACAATTTGTACTTTTTTCTTAAGGCATACTAGTACAAGT | |
| | | GGTAATTTTTGTACATTACACTAAATTATTAGCATTTGTT | |
| | | TTAGCATTACCTAATTTTTTTCCTGCTCCATGCAGACTGT | |
| | | TAGCTTTTACCTTAAATGCTTATTTTAAAATGACAGTGGA | |
| | | AGTTTTTTTTTCCTCTAAGTGCCAGTATTCCCAGAGTTTT | |
| | | GGTTTTTGAACTAGCAATGCCTGTGAAAAAGAAACTGAAT | |
| | | ACCTAAGATTTCTGTCTTGGGGTTTTTGGTGCATGCAGTT | |
| | | GATTACTTCTTATTTTTCTTACCAATTGTGAATGTTGGTG | |
| | | TGAAACAAATTAATGAAGCTTTTGAATCATCCCTATTCTG | |
| | | TGTTTTATCTAGTCACATAAATGGATTAATTACTAATTTC | |
| | | AGTTGAGACCTTCTAATTGGTTTTTACTGAAACATTGAGG | |
| | | GAACACAAATTTATGGGCTTCCTGATGATGATTCTTCTAG | |
| | | GCATCATGTCCTATAGTTTGTCATCCCTGATGAATGTAAA | |
| | | GTTACACTGTTCACAAAGGTTTTGTCTCCTTTCCACTGCT | |
| | | ATTAGTCATGGTCACTCTCCCCAAAATATTATATTTTTTC | |
| | | TATAAAAAGAAAAAAATGGAAAAAAATTACAAGGCAATGG | |
| | | AAACTATTATAAGGCCATTTCCTTTTCACATTAGATAAAT | |
| | | TACTATAAAGACTCCTAATAGCTTTTCCTGTTAAGGCAGA | |
| | | CCCAGTATGAAATGGGGATTATTATAGCAACCATTTTGGG | |
| | | GCTATATTTACATGCTACTAAATTTTTATAATAATTGAAA | |
| | | AGATTTTAACAAGTATAAAAAATTCTCATAGGAATTAAAT | |
| | | GTAGTCTCCCTGTGTCAGACTGCTCTTTCATAGTATAACT | |
| | | TTAAATCTTTTCTTCAACTTGAGTCTTTGAAGATAGTTTT | |
| | | AATTCTGCTTGTGACATTAAAAGATTATTTGGGCCAGTTA | |
| | | TAGCTTATTAGGTGTTGAAGAGACCAAGGTTGCAAGGCCA | |
| | | GGCCCTGTGTGAACCTTTGAGCTTTCATAGAGAGTTTCAC | |
| | | AGCATGGACTGTGTCCCCACGGTCATCCAGTGTTGTCATG | |
| | | CATTGGTTAGTCAAAATGGGGAGGGACTAGGGCAGTTTGG | |
| | | ATAGCTCAACAAGATACAATCTCACTCTGTGGTGGTCCTG | |
| | | CTGACAAATCAAGAGCATTGCTTTTGTTTCTTAAGAAAAC | |
| | | AAACTCTTTTTTAAAAATTACTTTTAAATATTAACTCAAA | |
| | | AGTTGAGATTTTGGGGTGGTGGTGTGCCAAGACATTAATT | |
| | | TTTTTTTTAAACAATGAAGTGAAAAAGTTTTACAATCTCT | |
| | | AGGTTTGGCTAGTTCTCTTAACACTGGTTAAATTAACATT | |
| | | GCATAAACACTTTTCAAGTCTGATCCATATTTAATAATGC | |
| | | TTTAAAATAAAAATAAAAACAATCCTTTTGATAAATTTAA | |
| | | AATGTTACTTATTTTAAAATAAATGAAGTGAGATGGCATG | |
| | | GTGAGGTGAAAGTATCACTGGACTAGGAAGAAGGTGACTT | |
| | | AGGTTCTAGATAGGTGTCTTTTAGGACTCTGATTTTGAGG | |
| | | ACATCACTTACTATCCATTTCTTCATGTTAAAAGAAGTCA | |
| | | TCTCAAACTCTTAGTTTTTTTTTTTTACAACTATGTAATT | |
| | | TATATTCCATTTACATAAGGATACACTTATTTGTCAAGCT | |
| | | CAGCACAATCTGTAAATTTTTAACCTATGTTACACCATCT | |
| | | TCAGTGCCAGTCTTGGGCAAAATTGTGCAAGAGGTGAAGT | |
| | | TTATATTTGAATATCCATTCTCGTTTTAGGACTCTTCTTC | |
| | | CATATTAGTGTCATCTTGCCTCCCTACCTTCCACATGCCC | |
| | | CATGACTTGATGCAGTTTTAATACTTGTAATTCCCCTAAC | |
| | | CATAAGATTTACTGCTGCTGTGGATATCTCCATGAAGTTT | |
| | | TCCCACTGAGTCACATCAGAAATGCCCTACATCTTATTTC | |
| | | CTCAGGGCTCAAGAGAATCTGACAGATACCATAAAGGGAT | |
| | | TTGACCTAATCACTAATTTTCAGGTGGTGGCTGATGCTTT | |
| | | GAACATCTCTTTGCTGCCCAATCCATTAGCGACAGTAGGA | |
| | | TTTTTCAAACCTGGTATGAATAGACAGAACCCTATCCAGT | |
| | | GGAAGGAGAATTTAATAAAGATAGTGCTGAAAGAATTCCT | |
| | | TAGGTAATCTATAACTAGGACTACTCCTGGTAACAGTAAT | |
| | | ACATTCCATTGTTTTAGTAACCAGAAATCTTCATGCAATG | |
| | | AAAAATACTTTAATTCATGAAGCTTACTTTTTTTTTTTGG | |
| | | TGTCAGAGTCTCGCTCTTGTCACCCAGGCTGGAATGCAGT | |
| | | GGCGCCATCTCAGCTCACTGCAACCTCCATCTCCCAGGTT | |
| | | CAAGCGATTCTCGTGCCTCGGCCTCCTGAGTAGCTGGGAT | |
| | | TACAGGCGTGTGCCACTACACTCAACTAATTTTTGTATTT | |
| | | TTAGGAGAGACGGGGTTTCACCCTGTTGGCCAGGCTGGTC | |
| | | TCGAACTCCTGACCTCAAGTGATTCACCCACCTTGGCCTC | |
| | | ATAAACCTGTTTTGCAGAACTCATTTATTCAGCAAATATT | |
| | | TATTGAGTGCCTACCAGATGCCAGTCACCGCACAAGGCAC | |
| | | TGGGTATATGGTATCCCCAAACAAGAGACATAATCCCGGT | |
| | | CCTTAGGTAGTGCTAGTGTGGTCTGTAATATCTTACTAAG | |
| | | GCCTTTGGTATACGACCCAGAGATAACACGATGCGTATTT | |
| | | TAGTTTTGCAAAGAAGGGGTTTGGTCTCTGTGCCAGCTCT | |
| | | ATAATTGTTTTGCTACGATTCCACTGAAACTCTTCGATCA | |
| | | AGCTACTTTATGTAAATCACTTCATTGTTTTAAAGGAATA | |
| | | AACTTGATTATATTGTTTTTTTATTTGGCATAACTGTGAT | |
| | | TCTTTTAGGACAATTACTGTACACATTAAGGTGTATGTCA | |
| | | GATATTCATATTGACCCAAATGTGTAATATTCCAGTTTTC | |
| | | TCTGCATAAGTAATTAAAATATACTTAAAAATTAATAGTT | |
| | | TTATCTGGGTACAAATAAACAGGTGCCTGAACTAGTTCAC | |
| | | AGACAAGGAAACTTCTATGTAAAAATCACTATGATTTCTG | |
| | | AATTGCTATGTGAAACTACAGATCTTTGGAACACTGTTTA | |
| | | GGTAGGGTGTTAAGACTTACACAGTACCTCGTTTCTACAC | |
| | | AGAGAAAGAAATGGCCATACTTCAGGAACTGCAGTGCTTA | |
| | | TGAGGGGATATTTAGGCCTCTTGAATTTTTGATGTAGATG | |
| | | GGCATTTTTTTAAGGTAGTGGTTAATTACCTTTATGTGAA | |
| | | CTTTGAATGGTTTAACAAAAGATTTGTTTTTGTAGAGATT | |
| | | TTAAAGGGGGAGAATTCTAGAAATAAATGTTACCTAATTA | |
| | | TTACAGCCTTAAAGACAAAAATCCTTGTTGAAGTTTTTTT | |
| | | AAAAAAAGCTAAATTACATAGACTTAGGCATTAACATGTT | |
| | | TGTGGAAGAATATAGCAGACGTATATTGTATCATTTGAGT | |
| | | GAATGTTCCCAAGTAGGCATTCTAGGCTCTATTTAACTGA | |
| | | GTCACACTGCATAGGAATTTAGAACCTAACTTTTATAGGT | |
| | | TATCAAAACTGTTGTCACCATTGCACAATTTTGTCCTAAT | |
| | | ATATACATAGAAACTTTGTGGGGCATGTTAAGTTACAGTT | |
| | | TGCACAAGTTCATCTCATTTGTATTCCATTGATTTTTTTT | |
| | | TTCTTCTAAACATTTTTTCTTCAAACAGTATATAACTTTT | |
| | | TTTAGGGGATTTTTTTTTAGACAGCAAAAACTATCTGAAG | |
| | | ATTTCCATTTGTCAAAAAGTAATGATTTCTTGATAATTGT | |
| | | GTAGTAATGTTTTTTAGAACCCAGCAGTTACCTTAAAGCT | |
| | | GAATTTATATTTAGTAACTTCTGTGTTAATACTGGATAGC | |
| | | ATGAATTCTGCATTGAGAAACTGAATAGCTGTCATAAAAT | |
| | | GAAACTTTCTTTCTAAAGAAAGATACTCACATGAGTTCTT | |
| | | GAAGAATAGTCATAACTAGATTAAGATCTGTGTTTTAGTT | |
| | | TAATAGTTTGAAGTGCCTGTTTGGGATAATGATAGGTAAT | |
| | | TTAGATGAATTTAGGGGAAAAAAAAGTTATCTGCAGATAT | |
| | | GTTGAGGGCCCATCTCTCCCCCCACACCCCCACAGAGCTA | |
| | | ACTGGGTTACAGTGTTTTATCCGAAAGTTTCCAATTCCAC | |
| | | TGTCTTGTGTTTTCATGTTGAAAATACTTTTGCATTTTTC | |
| | | CTTTGAGTGCCAATTTCTTACTAGTACTATTTCTTAATGT | |
| | | AACATGTTTACCTGGAATGTATTTTAACTATTTTTGTATA | |
| | | GTGTAAACTGAAACATGCACATTTTGTACATTGTGCTTTC | |
| | | TTTTGTGGGACATATGCAGTGTGATCCAGTTGTTTTCCAT | |
| | | CATTTGGTTGCGCTGACCTAGGAATGTTGGTCATATCAAA | |
| | | CATTAAAAATGACCACTCTTTTAATTGAAATTAACTTTTA | |
| | | AATGTTTATAGGAGTATGTGCTGTGAAGTGATCTAAAATT | |
| | | TGTAATATTTTTGTCATGAACTGTACTACTCCTAATTATT | |
| | | GTAATGTAATAAAAATAGTTACAGTGACTATGAGTGTGTA | |
| | | TTTATTCATGAAATTTGAACTGTTTGCCCCGAAATGGATA | |
| | | TGGAATACTTTATAAGCCATAGACACTATAGTATACCAGT | |
| | | GAATCTTTTATGCAGCTTGTTAGAAGTATCCTTTATTTCT | |
| | | AAAAGGTGCTGTGGATATTATGTAAAGGCGTGTTTGCTTA | |
| | | AACTTAAAACCATATTTAGAAGTAGATGCAAAACAAATCT | |
| | | GCCTTTATGACAAAAAAATAGGATAACATTATTTATTTAT | |
| | | TTCCTTTTATCAAAGAAGGTAATTGATACACAACAGGTGA | |
| | | CTTGGTTTTAGGCCCAAAGGTAGCAGCAGCAACATTAATA | |
| | | ATGGAAATAATTGAATAGTTAGTTATGTATGTTAATGCCA | |
| | | GTCACCAGCAGGCTATTTCAAGGTCAGAAGTAATGACTCC | |
| | | ATACATATTATTTATTTCTATAACTACATTTAAATCATTA | |
| | | CCAGG | |
| LEO1 | NM_138792.3 | CGTAAAGAGAGGCCGGGAGCTGCCCCTAACCGAGGCAGCA | 21 |
| | | GCGGACGTGAGCGATAATGGCGGATATGGAGGATCTCTTC | |
| | | GGGAGCGACGCCGACAGCGAAGCTGAGCGTAAAGATTCTG | |
| | | ATTCTGGATCTGACTCAGATTCTGATCAAGAGAATGCTGC | |
| | | CTCTGGCAGTAATGCCTCTGGAAGTGAAAGTGATCAGGAT | |
| | | GAAAGAGGTGATTCAGGACAACCAAGTAATAAGGAACTGT | |
| | | TTGGAGATGACAGTGAGGACGAGGGAGCTTCACATCATAG | |
| | | TGGTAGTGATAATCACTCTGAAAGATCAGACAATAGATCA | |
| | | GAAGCTTCTGAGCGTTCTGACCATGAGGACAATGACCCCT | |
| | | CAGATGTAGATCAGCACAGTGGATCAGAAGCCCCTAATGA | |
| | | TGATGAAGACGAAGGTCATAGATCGGATGGAGGGAGCCAT | |
| | | CATTCAGAAGCAGAAGGTTCTGAAAAAGCACATTCAGATG | |
| | | ATGAAAAATGGGGCAGAGAAGATAAAAGTGACCAGTCAGA | |
| | | TGATGAAAAGATACAAAATTCTGATGATGAGGAGAGGGCA | |
| | | CAAGGATCTGATGAAGATAAGCTGCAGAATTCTGACGATG | |
| | | ATGAGAAAATGCAGAACACAGATGATGAGGAGAGGCCTCA | |
| | | GCTTTCCGATGATGAGAGACAACAGCTATCTGAGGAGGAA | |
| | | AAGGCTAATTCTGATGATGAACGGCCGGTAGCTTCTGATA | |
| | | ATGATGATGAGAAACAGAATTCTGATGATGAAGAACAACC | |
| | | ACAGCTGTCTGATGAAGAGAAAATGCAAAATTCTGATGAT | |
| | | GAAAGGCCACAGGCCTCAGATGAAGAACACAGGCATTCAG | |
| | | ATGATGAAGAGGAACAGGATCATAAATCAGAATCTGCAAG | |
| | | AGGCAGTGATAGTGAAGATGAAGTTTTACGAATGAAACGC | |
| | | AAGAATGCGATTGCATCTGATTCAGAAGCGGATAGTGACA | |
| | | CTGAGGTGCCAAAAGATAATAGTGGAACCATGGATTTATT | |
| | | TGGAGGTGCAGATGATATCTCTTCAGGGAGTGATGGAGAA | |
| | | GACAAACCACCTACTCCAGGACAGCCTGTTGATGAAAATG | |
| | | GATTGCCTCAGGATCAACAGGAAGAGGAGCCAATTCCTGA | |
| | | GACCAGAATAGAAGTAGAAATACCCAAAGTAAACACTGAT | |
| | | TTAGGAAACGACTTATATTTTGTTAAACTGCCCAACTTTC | |
| | | TCAGTGTAGAGCCCAGACCTTTTGATCCTCAGTATTATGA | |
| | | AGATGAATTTGAAGATGAAGAAATGCTGGATGAAGAAGGT | |
| | | AGAACCAGGTTAAAATTAAAGGTAGAAAATACTATAAGAT | |
| | | GGAGGATACGCCGAGATGAAGAAGGAAATGAAATTAAAGA | |
| | | AAGCAATGCTCGGATAGTCAAGTGGTCAGATGGAAGCATG | |
| | | TCCCTGCATTTAGGCAATGAAGTGTTTGATGTGTACAAAG | |
| | | CCCCACTGCAGGGCGACCACAATCATCTTTTTATAAGACA | |
| | | AGGTACTGGTCTACAGGGACAAGCAGTCTTTAAAACGAAA | |
| | | CTCACCTTCAGACCTCACTCTACGGACAGTGCCACACATA | |
| | | GAAAGATGACTCTGTCACTTGCAGATAGGTGTTCAAAGAC | |
| | | ACAGAAGATTAGAATCTTGCCAATGGCTGGTCGTGATCCT | |
| | | GAATGCCAACGCACAGAAATGATTAAGAAAGAAGAAGAAC | |
| | | GTTTGAGGGCTTCCATACGTAGGGAATCTCAGCAGCGCCG | |
| | | AATGAGAGAGAAACAGCACCAGCGGGGGCTGAGCGCCAGT | |
| | | TACCTGGAACCTGATCGATACGATGAGGAGGAGGAAGGCG | |
| | | AGGAGTCCATCAGCTTGGCTGCCATTAAAAACCGATATAA | |
| | | AGGGGGCATTCGAGAGGAACGAGCCAGAATCTATTCATCA | |
| | | GACAGTGATGAGGGATCAGAAGAAGATAAAGCTCAAAGAT | |
| | | TACTCAAAGCAAAGAAACTTACCAGTGATGAGGAAGGTGA | |
| | | ACCTTCCGGAAAGAGAAAAGCAGAAGATGATGATAAAGCA | |
| | | AATAAAAAGCATAAGAAGTATGTGATCAGCGATGAAGAGG | |
| | | AAGAAGATGATGATTGAAGTATGAAATATGAAAACATTTT | |
| | | ATATATTTTATTGTACAGTTATAAATATGTAAACATGAGT | |
| | | TATTTTGATTGAAATGAATCGATTTGCTTTTGTGTAATTT | |
| | | TAATTGTAATAAAACAATTTAAAAGCAAAAAAAAAAAAAA | |
| | | AA | |
| MORF4L2 | NM_001142418.1 | TTGATTATGGAACATTCTAAAACTTAGACAAGACGATTGT | 22 |
| | | GATTGGCTGAAGGGCATACGCCCTCCTCCAGGGTGACGTG | |
| | | TCTGCCTATGGATATCAGTTGCCAGAGAAACCTGGCTTTA | |
| | | CTATGGCGGTTGGAGGAACGGCAGTGATCACACGTCGGCT | |
| | | GCTGGGAAGATCTGGATTCTCGTTTCAGGTCACCATCAGA | |
| | | AAAGCTAAGTTTGCTGTATAGTGAGGATCAGGAGATCTGA | |
| | | TCCTGATTGCAGAACCTTCCCTGATTACAGAATCTTGGGA | |
| | | TTGTTGAGAGGATTACATGTAAAGTACCAGGACAGTGCAT | |
| | | GGCACATATGATTTCACAAAAGTTCATCTTCATTGCAGAT | |
| | | ACCTGCCTTTCTTTCTAGGTTGTATCTCCCACTTCACCCT | |
| | | TCTAGACCATCCCAGAAGATCTATAAGATTTCATCTGGGA | |
| | | AATCACTAGGAGTTCTTGGAAGGGAAAGAAGGAAGATTGT | |
| | | TGGTTGGAATAAAAACAGGGTTGAATGAGTTCCAGAAAGC | |
| | | AGGGTTCTCAACCTCGTGGACAGCAATCTGCAGAAGAAGA | |
| | | GAACTTCAAAAAACCAACTAGAAGCAACATGCAGAGAAGT | |
| | | AAAATGAGAGGGGCCTCCTCAGGAAAGAAGACAGCTGGTC | |
| | | CACAGCAGAAAAATCTTGAACCAGCTCTCCCAGGAAGATG | |
| | | GGGTGGTCGCTCTGCAGAGAACCCCCCTTCAGGATCCGTG | |
| | | AGGAAGACCAGAAAGAACAAGCAGAAGACTCCTGGAAACG | |
| | | GAGATGGTGGCAGTACCAGCGAAGCACCTCAGCCCCCTCG | |
| | | GAAGAAAAGGGCCCGGGCAGACCCCACTGTTGAAAGTGAG | |
| | | GAGGCGTTTAAGAATAGAATGGAGGTTAAAGTGAAGATTC | |
| | | CTGAAGAATTAAAACCATGGCTTGTTGAGGACTGGGACTT | |
| | | AGTTACCAGGCAGAAGCAGCTGTTTCAACTCCCTGCCAAG | |
| | | AAAAATGTAGATGCAATTCTGGAGGAGTATGCAAATTGCA | |
| | | AGAAATCGCAGGGAAATGTTGATAATAAGGAATATGCGGT | |
| | | TAATGAAGTTGTGGCAGGAATAAAAGAATATTTCAATGTG | |
| | | ATGTTGGGCACTCAGCTGCTCTACAAATTTGAGAGGCCCC | |
| | | AGTATGCTGAAATCCTCTTGGCTCACCCTGATGCTCCAAT | |
| | | GTCCCAGGTTTATGGAGCACCACACCTACTGAGATTATTT | |
| | | GTAAGAATTGGAGCAATGTTGGCCTATACGCCCCTTGATG | |
| | | AGAAAAGCCTTGCATTATTGTTGGGCTATTTGCATGATTT | |
| | | CCTAAAATATCTGGCAAAGAATTCTGCATCTCTCTTTACT | |
| | | GCCAGTGATTACAAAGTGGCTTCTGCTGAGTACCACCGCA | |
| | | AAGCCCTGTGAGCGTCTACAGACAGCTCACCATTTTTGTC | |
| | | CTGTATCTGTAAACACTTTTTGTTCTTAGTCTTTTTCTTG | |
| | | TAAAATTGATGTTCTTTAAAATCGTTAATGTATAACAGGG | |
| | | CTTATGTTTCAGTTTGTTTTCCGTTCTGTTTTAAACAGAA | |
| | | AATAAAAGGAGTGTAAGCTCCTTTTCTCATTTCAAAGTTG | |
| | | CTACCAGTGTATGCAGTAATTAGAACAAAGAAGAAACATT | |
| | | CAGTAGAACATTTTATTGCCTAGTTGACAACATTGCTTGA | |
| | | ATGCTGGTGGTTCCTATCCCTTTGACACTACACAATTTTC | |
| | | TAATATGTGTTAATGCTATGTGACAAAACGCCCTGATTCC | |
| | | TAGTGCCAAAGGTTCAACTTAATGTATATACCTGAAAACC | |
| | | CATGCATTTGTGCTCTTTTTTTTTTTTTATGGTGCTTGAA | |
| | | GTAAAACAGCCCATCCTCTGCAAGTCCATCTATGTTGTTC | |
| | | TTAGGCATTCTATCTTTGCTCAAATTGTTGAAGGATGGTG | |
| | | ATTTGTTTCATGGTTTTTGTATTTGAGTCTAATGCACGTT | |
| | | CTAACATGATAGAGGCAATGCATTATTGTGTAGCCACGGT | |
| | | TTTCTGGAAAAGTTGATATTTTAGGAATTGTATTTCAGAT | |
| | | CTTAAATAAAATTTGTTTCTAAATTTCAAAGCAAAAAAAA | |
| | | AAAAAAA | |
| NAP1L1 | NM_139207.2 | AAAAGATATGGTGGGGTGCTTAACAGAGGAGGTTAGACAC | 23 |
| | | CGGCGGGAACCAGAGGAGCCCAAGCGCGGCGCCTGGGCCT | |
| | | CGGGGCTGCAGGAGTCCTCGGTGGGGGTATGGAGGTCGCC | |
| | | GGGGAAGGAGGACGGTTCAGTTGCTAGGCAACCCGGCCTG | |
| | | GACCCGCCTCTCGCTCGCGTTGCTGGGAGACTACAAGGCC | |
| | | GGGAGGAGGGCGGCGAAAGGGCCCTACGTGCTGACGCTAA | |
| | | TTGTATATGAGCGCGAGCGGCGGGCTCTTGGGTCTTTTTT | |
| | | AGCGCCATCTGCTCGCGGCGCCGCCTCCTGCTCCTCCCGC | |
| | | TGCTGCTGCCGCTGCCGCCCTGAGTCACTGCCTGCGCAGC | |
| | | TCCGGCCGCCTGGCTCCCCATACTAGTCGCCGATATTTGG | |
| | | AGTTCTTACAACATGGCAGACATTGACAACAAAGAACAGT | |
| | | CTGAACTTGATCAAGATTTGGATGATGTTGAAGAAGTAGA | |
| | | AGAAGAGGAAACTGGTGAAGAAACAAAACTCAAAGCACGT | |
| | | CAGCTAACTGTTCAGATGATGCAAAATCCTCAGATTCTTG | |
| | | CAGCCCTTCAAGAAAGACTTGATGGTCTGGTAGAAACACC | |
| | | AACAGGATACATTGAAAGCCTGCCTAGGGTAGTTAAAAGA | |
| | | CGAGTGAATGCTCTCAAAAACCTGCAAGTTAAATGTGCAC | |
| | | AGATAGAAGCCAAATTCTATGAGGAAGTTCACGATCTTGA | |
| | | AAGGAAGTATGCTGTTCTCTATCAGCCTCTATTTGATAAG | |
| | | CGATTTGAAATTATTAATGCAATTTATGAACCTACGGAAG | |
| | | AAGAATGTGAATGGAAACCAGATGAAGAAGATGAGATTTC | |
| | | GGAGGAATTGAAAGAAAAGGCCAAGATTGAAGATGAGAAA | |
| | | AAAGATGAAGAAAAAGAAGACCCCAAAGGAATTCCTGAAT | |
| | | TTTGGTTAACTGTTTTTAAGAATGTTGACTTGCTCAGTGA | |
| | | TATGGTTCAGGAACACGATGAACCTATTCTGAAGCACTTG | |
| | | AAAGATATTAAAGTGAAGTTCTCAGATGCTGGCCAGCCTA | |
| | | TGAGTTTTGTCTTAGAATTTCACTTTGAACCCAATGAATA | |
| | | TTTTACAAATGAAGTGCTGACAAAGACATACAGGATGAGG | |
| | | TCAGAACCAGATGATTCTGATCCCTTTTCTTTTGATGGAC | |
| | | CAGAAATTATGGGTTGTACAGGGTGCCAGATAGATTGGAA | |
| | | AAAAGGAAAGAATGTCACTTTGAAAACTATTAAGAAGAAG | |
| | | CAGAAACACAAGGGACGTGGGACAGTTCGTACTGTGACTA | |
| | | AAACAGTTTCCAATGACTCTTTCTTTAACTTTTTTGCCCC | |
| | | TCCTGAAGTTCCTGAGAGTGGAGATCTGGATGATGATGCT | |
| | | GAAGCTATCCTTGCTGCAGACTTCGAAATTGGTCACTTTT | |
| | | TACGTGAGCGTATAATCCCAAGATCAGTGTTATATTTTAC | |
| | | TGGAGAAGCTATTGAAGATGATGATGATGATTATGATGAA | |
| | | GAAGGTGAAGAAGCGGATGAGGAAGGGGAAGAAGAAGGAG | |
| | | ATGAGGAAAATGATCCAGACTATGACCCAAAGAAGGATCA | |
| | | AAACCCAGCAGAGTGCAAGCAGCAGTGAAGCAGGATGTAT | |
| | | GTGGCCTTGAGGATAACCTGCACTGGTCTACCTTCTGCTT | |
| | | CCCTGGAAAGGATGAATTTACATCATTTGACAAGCCTATT | |
| | | TTCAAGTTATTTGTTGTTTGTTTGCTTGTTTTTGTTTTTG | |
| | | CAGCTAAAATAAAAATTTCAAATACAATTTTAGTTCTTAC | |
| | | AAGATAATGTCTTAATTTTGTACCAATTCAGGTAGAAGTA | |
| | | GAGGCCTACCTTGAATTAAGGGTTATACTCAGTTTTTAAC | |
| | | ACATTGTTGAAGAAAAGGTACCAGCTTTGGAACGAGATGC | |
| | | TATACTAATAAGCAAGTGTAAAAAAAAAAAAAAAAGAGGA | |
| | | AGAAAATCTTAAGTGATTGATGCTGTTTTCTTTTAAAAAA | |
| | | AAAAAAAAAAATTCATTTTCTTTGGGTTAGAGCTAGAGAG | |
| | | AAGGCCCCAAGCTTCTATGGTTTCTTCTAATTCTTATTGC | |
| | | TTAAAGTATGAGTATGTCACTTACCCGTGCTTCTGTTTAC | |
| | | TGTGTAATTAAAATGGGTAGTACTGTTTACCTAACTACCT | |
| | | CATGGATGTGTTAAGGCATATTGAGTTAAATCTCATATAA | |
| | | TGTTTCTCAATCTTGTTAAAAGCTCAAAATTTTGGGCCTA | |
| | | TTTGTAATGCCAGTGTGACACTAAGCATTTTGTTCACACC | |
| | | ACGCTTTGATAACTAAACTGGAAAACAAAGGTGTTAAGTA | |
| | | CCTCTGTTCTGGATCTGGGCAGTCAGCACTCTTTTTAGAT | |
| | | CTTTGTGTGGCTCCTATTTTTATAGAAGTGGAGGGATGCA | |
| | | CTATTTCACAAGGTCCAAGATTTGTTTTCAGATATTTTTG | |
| | | ATGACTGTATTGTAAATACTACAGGGATAGCACTATAGTA | |
| | | TTGTAGTCATGAGACTTAAAGTGGAAATAAGACTATTTTT | |
| | | GACAAAAGATGCCATTAAATTTCAGACTGTAGAGCCACAT | |
| | | TTACAATACCTCAGGCTAATTACTGTTAATTTTGGGGTTG | |
| | | AACTTTTTTTTGACAGTGAGGGTGGATTATTGGATTGTCA | |
| | | TTAGAGGAAGGTCTAGATTTCCTGCTCTTAATAAAATTAC | |
| | | ATTGAATTGATTTTTAGAGGTAATGAAAACTTCCTTTCTG | |
| | | AGAAGTTAGTGTTAAGGTCTTGGAATGTGAACACATTGTT | |
| | | TGTAGTGCTATCCATTCCTCTCCTGAGATTTTAACTTACT | |
| | | ACTGGAAATCCTTAACCAATTATAATAGCTTTTTTTCTTT | |
| | | ATTTTCAAAATGATTTCCTTTGCTTTGATTAGACACTATG | |
| | | TGCTTTTTTTTTTTAACCATAGTTCATCGAAATGCAGCTT | |
| | | TTTCTGAACTTCAAAGATAGAATCCCATTTTTAATGAACT | |
| | | GAAGTAGCAAAATCATCTTTTTCATTCTTTAGGAAATAGC | |
| | | TATTGCCAAAGTGAAGGTGTAGATAATACCTAGTCTTGTT | |
| | | ACATAAAGGGGATGTGGTTTGCAGAAGAATTTTCTTTATA | |
| | | AAATTGAAGTTTTAAGGGACGTCAGTGTTTATGCCATTTT | |
| | | TCCAGTTCCAAAATGATTCCATTCCATTCTAGAAATTTGA | |
| | | AGTATGTAACCTGAAATCCTTAATAAAATTTGGATTTAAT | |
| | | TTTATAAAATGTACTGGTGATATTTTGGGTGTTTTTTTTT | |
| | | AAATGAATGTATATACTTTTTTTTTGAAGAGTGGAGAGTA | |
| | | GTGATGTCTAGAGGGAGCTATTTTGTGCTGAGGCCACTAT | |
| | | GTTCTGTAAATATATAATTTTAAGAGCAACCTCACAATCC | |
| | | CTGCTAAGTGGAGTTTATTATTTGAAGACTAAAATGGAAT | |
| | | TCCATAGTTCCTGATAGGTTATATTCTGGGTTATTATTCT | |
| | | GAGTTATCTACAAACATTTTTGAGATTTGTCTTTACACTC | |
| | | TGATTGTAGTTTCCAGCAGCCCATGCACACTGCCAAGTAA | |
| | | GTCTCATTTTTTCCTGTTAGAAATGGTGAAATATCATATA | |
| | | ATCACTTATAAAGAAAACTGATATGAAAAAATTTTAGAGT | |
| | | TGTTTGCTTTATGGTCACTCAAGTAGGGTAAGTGTTCCAC | |
| | | AAATTCCACAAGTTGATAGTTTAACATGGATGTCTGAAAG | |
| | | CCACATATATAATTTCTTAGGATTCTTAAATTAGTAAATC | |
| | | TAGCTTACTGAAGCAGTATTAGCATCACTATTTTAGATTG | |
| | | CAAAAATACCTTAATTGTGTGGAACTGGCTTGTAGAGTGG | |
| | | TACTTAAGAAAAATGGGATTCTACCTCTATTTCTGTTTTA | |
| | | GCACACTTAATCAGGAAAGGATATATTAACTTTCATAAAA | |
| | | ATATTTTTGTTGTGTGAATAGGTTAATGATATGGTAAGGC | |
| | | CCCTAAAATAACTGAATTAATTGTTTATTGTAATTGTAGG | |
| | | CCATTCCCATTATTAAAAATAAAGACAAAACTTGAAGTAA | |
| | | CTGAAAATCTTATCGTGCTATGTAGAAATATTGAACTAAT | |
| | | ATTCAAATATTTGAATGCTTTGGTTTCAGGGATTGGTTTA | |
| | | AAATTGGAGTCCTTTTTTATGGGTTAGTCTTACAAAAATT | |
| | | TAAGCCTTTATATTTTTGACTTTAAATCAAAACAAATGTT | |
| | | ATTTTAAATGTACAGAATAGATTGGTAGTGCAGAAGAGTG | |
| | | TAAGTTCTTCATAGGAGCTTTAGAAAAGAGAAATATGTGC | |
| | | TAATTCAGTTTTTTTTTAATCTGCACTGTACATATATACT | |
| | | TGGTAATTATGAGCTTGATTTTGTTTTTGGAAATATGTGT | |
| | | TCATAATTTAGGTAATTTGCTACTTAAAGCACTAAGTCTC | |
| | | TGATACCTGAAAAGTACATGTAAATGGTGATGGTGAAATA | |
| | | ATACTGCAGTTAACTTAATAGATGTATACTGGTGATTTTT | |
| | | GTATGCTGGATTAAAACTCCAGATATTAAAATATAACCTG | |
| | | GATAAAAAGCC | |
| NOL3 | NM_001185057.2 | GGCATTCAGAGAGTAGATGCCAGTCCTGGGAAAGGCAGGG | 24 |
| | | GAGGAGAGGAGAGCCACGGCTGACGCTTGGGGACAGAAGG | |
| | | AGGAGCCTGAGGAGGAGACAGGACAGAGCGTCTGGAGAGG | |
| | | CAGGAGGACACCGAGTTCCCCGTGTTGGCCTCCAGGTCCT | |
| | | GTGCTTGCGGAGCCGTCCGGCGGCTGGGATCGAGCCCCGA | |
| | | CAATGGGCAACGCGCAGGAGCGGCCGTCAGAGACTATCGA | |
| | | CCGCGAGCGGAAACGCCTGGTCGAGACGCTGCAGGCGGAC | |
| | | TCGGGACTGCTGTTGGACGCGCTGCTGGCGCGGGGCGTGC | |
| | | TCACCGGGCCAGAGTACGAGGCATTGGATGCACTGCCTGA | |
| | | TGCCGAGCGCAGGGTGCGCCGCCTACTGCTGCTGGTGCAG | |
| | | GGCAAGGGCGAGGCCGCCTGCCAGGAGCTGCTACGCTGTG | |
| | | CCCAGCGTACCGCGGGCGCGCCGGACCCCGCTTGGGACTG | |
| | | GCAGCACGCTACCGGGACCGCAGCTATGACCCTCCATGCC | |
| | | CAGGCCACTGGACGCCGGAGGCACCCGGCTCGGGGACCAC | |
| | | ATGCCCCGGGTTGCCCAGAGCTTCAGACCCTGACGAGGCC | |
| | | GGGGGCCCTGAGGGCTCCGAGGCGGTGCAATCCGGGACCC | |
| | | CGGAGGAGCCAGAGCCAGAGCTGGAAGCTGAGGCCTCTAA | |
| | | AGAGGCTGAACCGGAGCCGGAGCCAGAGCCAGAGCTGGAA | |
| | | CCCGAGGCTGAAGCAGAACCAGAGCCGGAACTGGAGCCAG | |
| | | AACCGGACCCAGAGCCCGAGCCCGACTTCGAGGAAAGGGA | |
| | | CGAGTCCGAAGATTCCTGAAGGCCAGAGCTCTGACAGGCG | |
| | | GTGCCCCGCCCATGCTGGATAGGACCTGGGATGCTGCTGG | |
| | | AGCTGAATCGGATGCCACCAAGGCTCGGTCCAGCCCAGTA | |
| | | CCGCTGGAAGTGAATAAACTCCGGAGGGTCGGACGGGACC | |
| | | TGGGCTCTCTCCACGATTCTGGCTGTTTGCCCAGGAACTT | |
| | | AGGGTGGGTACCTCTGAGTCCCAGGGACCTGGGCAGGCCC | |
| | | AAGCCCACCACGAGCATCATCCAGTCCTCAGCCCTAATCT | |
| | | GCCCTTAGGAGTCCAGGCTGCACCCTGGAGATCCCAAACC | |
| | | TAGCCCCCTAGTGGGACAAGGACCTGACCCTCCTGCCCGC | |
| | | ATACACAACCCATTTCCCCTGGTGAGCCACTTGGCAGCAT | |
| | | ATGTAGGTACCAGCTCAACCCCACGCAAGTTCCTGAGCTG | |
| | | AACATGGAGCAAGGGGAGGGTGACTTCTCTCCACATAGGG | |
| | | AGGGCTTAGAGCTCACAGCCTTGGGAAGTGAGACTAGAAG | |
| | | AGGGGAGCAGAAAGGGACCTTGAGTAGACAAAGGCCACAC | |
| | | ACATCATTGTCATTACTGTTTTAATTGTCTGGCTTCTCTC | |
| | | TGGACTGGGAGCTCAGTGAGGATTCTGACCAGTGACTTAC | |
| | | ACAAAAGGCGCTCTATACATATTATAATATATTCGCTTAC | |
| | | TAAATGAATAAGGACTTTCCAAAAAAAAAAAAAAAAAAAA | |
| | | AAAAAAAAAA | |
| NUDT3 | NM_006703.3 | GGTGCAGCCTTACGCCGCTGACGCATCGCGCCCAAGATGG | 25 |
| | | CGGCGCGGTCGTCGTCGGGGGTGGCGGCGGCAGAGGGGGC | |
| | | GGCGGCCCTGGCGGCAGCGGAGACGGCAGCCGTGACGGTG | |
| | | GCAGCGGCGGCGCGGGACCTGGGCCTGGGGGAATGAGGCG | |
| | | GCCGCGGCGGGCCAGCGGCGGAGCCGTGTAGCGGAGAAGC | |
| | | TCCCCCTCCCTGCTTCCCTTGGCCGAGCCGGGGGCGCGCG | |
| | | CGCACGCGGCCGTCCAGAGCGGGCTCCCCACCCCTCGACT | |
| | | CCTGCGACCCGCACCGCACCCCCACCCGGGCCCGGAGGAT | |
| | | GATGAAGCTCAAGTCGAACCAGACCCGCACCTACGACGGC | |
| | | GACGGCTACAAGAAGCGGGCCGCATGCCTGTGTTTCCGCA | |
| | | GCGAGAGCGAGGAGGAGGTGCTACTCGTGAGCAGTAGTCG | |
| | | CCATCCAGACAGATGGATTGTCCCTGGAGGAGGCATGGAG | |
| | | CCCGAGGAGGAGCCAAGTGTGGCAGCAGTTCGTGAAGTCT | |
| | | GTGAGGAGGCTGGAGTAAAAGGGACATTGGGAAGATTAGT | |
| | | TGGAATTTTTGAGAACCAGGAGAGGAAGCACAGGACGTAT | |
| | | GTCTATGTGCTCATTGTCACTGAAGTGCTGGAAGACTGGG | |
| | | AAGATTCAGTTAACATTGGAAGGAAGAGGGAATGGTTTAA | |
| | | AATAGAAGACGCCATAAAAGTGCTGCAGTATCACAAACCC | |
| | | GTGCAGGCATCATATTTTGAAACATTGAGGCAAGGCTACT | |
| | | CAGCCAACAATGGCACCCCAGTCGTGGCCACCACATACTC | |
| | | GGTTTCTGCTCAGAGCTCGATGTCAGGCATCAGATGACTG | |
| | | AAGACTTCCTGTAAGAGAAATGGAAATTGGAAACTAGACT | |
| | | GAAGTGCAAATCTTCCCTCTCACCCTGGCTCTTTCCACTT | |
| | | CTCACAGGCCTCCTCTTTCAAATAAGGCATGGTGGGCAGC | |
| | | AAAGAAAGGGTGTATTGATAATGTTGCTGTTTGGTGTTAA | |
| | | GTGATGGGGCTTTTTCTTCTGTTTTTATTGAGGGTGGGGG | |
| | | TTGGGTGTGTAATTTGTAAGTACTTTTGTGCATGATCTGT | |
| | | CCCTCCCTCTTCCCACCCCTGCAGTCCTCTGAAGAGAGGC | |
| | | CAACAGCCTTCCCCTGCCTTGGATTCTGAAGTGTTCCTGT | |
| | | TTGTCTTATCCTGGCCCTGGCCAGACGTTTTCTTTGATTT | |
| | | TTAATTTTTTTTTTTTATTAAAAGATACCAGTATGAGATG | |
| | | AAAACTTCCAATAATTTGTCCTATAATGTGCTGTACAGTT | |
| | | CAGTAGAGTGGTCACTTTCACTGCAGTATACATTTATCTA | |
| | | CACATTATATATCGGACATATAATATGTAAATAAATGACT | |
| | | TCTAGAAAGAGAAATTTGTTTAATTTTTCAAGGTTTTTTT | |
| | | CTCTTTTAATTTGGGCATTTCTAGAATTGAGAGCCTCACA | |
| | | ATTAACATACCTTTTTGTTTTCGATGCTAGTGGCTGGGCA | |
| | | GGTTGCCCTGTCCTTTCTCTATTTCCCAGTCATTGACTGT | |
| | | AGATATGGGAAGAGTTTAGCTACCTTCATAGTGCTCCCAG | |
| | | GACTCATGGCCTTTCCTTCTTTAAGCTGTATTTCCCTGCC | |
| | | CAGAAAGAAACAGGAAGAAACCTTTTTTTATTTTTTTATT | |
| | | TTTTTTTAACCAAGCAAGGAGCAAATGGCCTCAGCCCAGA | |
| | | TCTGTAAAAACAATGATAGAAATTGAATTCTGCCCCACAT | |
| | | GTTGACAGTAGAGTTGGAACTGGATTCTTGGGATTACTTA | |
| | | TCTAAAAAACTGGAGCATCAGGTCCATTTCTGTTCTGCTG | |
| | | GTTTGGAATCTTTTCCGTAATGCTATTTATTGCCAACAAT | |
| | | GGCCTCTCTTTGTGTCCATATATGCCTTACACCGTGCTGA | |
| | | CCTGGGTATCATCCATGTGCTCTGAAGCATCCAACTTTAC | |
| | | TTTGCAGGTGCATCAATGTAGTCCTGTCCCTGAACTGAGT | |
| | | AACCGTGTTCCTGAAAAGTACACTAGGGAAATTCACCTGC | |
| | | TTGCTTGTCTTTGTATTGGCATGGCACTTGTGATTGCACC | |
| | | ATGGAGCATGCTCAGAGCTATTAAATTGGTCTCCCATCTC | |
| | | CCACCAGGATATGAAAGGTCCATATGGGAGGCCACGTAAT | |
| | | CACTTATTACAGTGGTTACATAATACACTGGCTCACTGCA | |
| | | GACTCTCTTGTTTTTTGATACAGTTTCGTGCTGGCTTCAT | |
| | | TTGCCAATTGTGTTGTTTAGTTCGGAAGTAAGAGGGTCTT | |
| | | GAGATTGAGGGGTAGGGAGGGCTACACTGACTGATCCGTG | |
| | | GCTTAAGACAGGAGATTATCTCTGTACTCCAGTGGCATCT | |
| | | CCTTAGCCAAGATGTGAAATTAAAATCATAGTTCGCCTCA | |
| | | TTTAAAAATTCTAATAAAGCACTCAAACTTTGAAAAAAAA | |
| | | AAAAAAAAAA | |
| OAZ2 | NM_002537.3 | ATGCAGATGAGGCACTCGGGGGCGGGGCGGCGGCGGCGGC | 26 |
| | | GGCGGCGGTGGCGGCCGGGGAGGGTCAGTTGGAGGCAGGC | |
| | | GCTCGCTGAGGCAAAAGGAGGCGCTCGGCCCGCGGCCTGA | |
| | | CAGGGACTTAGCCCGCAGAGATCGACCCCGCGCGCGTGAC | |
| | | CCCACACCCACCCACTCATCCATCTATCCACTCCCTGCGC | |
| | | CGCCTCCTCCCACCCTGAGCAGAGCCGCCGAGGATGATAA | |
| | | ACACCCAGGACAGTAGTATTTTGCCTTTGAGTAACTGTCC | |
| | | CCAGCTCCAGTGCTGCAGGCACATTGTTCCAGGGCCTCTG | |
| | | TGGTGCTCCTGATGCCCCTCACCCACTGTCGAAGATCCCC | |
| | | GGTGGGCGAGGGGGCGGCAGGGATCCTTCTCTCTCAGCTC | |
| | | TAATATATAAGGACGAGAAGCTCACTGTGACCCAGGACCT | |
| | | CCCTGTGAATGATGGAAAACCTCACATCGTCCACTTCCAG | |
| | | TATGAGGTCACCGAGGTGAAGGTCTCTTCTTGGGATGCAG | |
| | | TCCTGTCCAGCCAGAGCCTGTTTGTAGAAATCCCAGATGG | |
| | | ATTATTAGCTGATGGGAGCAAAGAAGGATTGTTAGCACTG | |
| | | CTAGAGTTTGCTGAAGAGAAGATGAAAGTGAACTATGTCT | |
| | | TCATCTGCTTCAGGAAGGGCCGAGAAGACAGAGCTCCACT | |
| | | CCTGAAGACCTTCAGCTTCTTGGGCTTTGAGATTGTACGT | |
| | | CCAGGCCATCCCTGTGTCCCCTCTCGGCCAGATGTGATGT | |
| | | TCATGGTTTATCCCCTGGACCAGAACTTGTCCGATGAGGA | |
| | | CTAATAGTCATAGAGGATGCTTTACCCAAGAGCCACAGTG | |
| | | GGGGAAGAGGGGAAGTTAGGCAGCCCTGGGACAGACGAGA | |
| | | GGGCTCCTCGCTGTCTAGGGAAGGACACTGAGGGGCTCAG | |
| | | GGTGAGGGTTGCCTATTGTGTTCTCGGAGTTGACTCGTTG | |
| | | AAATTGTTTTCCATAAAGAACAGTATAAACATATTATTCA | |
| | | CATGTAATCACCAATAGTAAATGAAGATGTTTATGAACTG | |
| | | GCATTAGAAGCTTTCTAAACTGCGCTGTGTGATGTGTTCT | |
| | | ATCTAGCCTAGGGGAGGACATTGCCTAGAGGGGGAGGGAC | |
| | | TGTCTGGGTTCAGGGGCATGGCCTGGAGGGCTGGTGGGCA | |
| | | GCACTGTCAGGCTCAGGTTTCCCTGCTGTTGGCTTTCTGT | |
| | | TTTGGTTATTAAGACTTGTGTATTTTCTTTCTTTGCTTCC | |
| | | TGTCACCCCAGGGGCTCCTGAGTATAGGCTTTTCAGTCCC | |
| | | TGGGCAGTGTCCTTGAGTTGTTTTTTGACACTCTTACCTG | |
| | | GGCTTCTCTGTGTGCATTTGCGTCTGGCCTGGAGTAAGCA | |
| | | GGTCCGACCCCTCCTTCTTTACAGCTTAGTGTTATTCTGG | |
| | | CATTTGGTTAAGCTGGCTTAATCTGTTTAATGTTATCAGT | |
| | | ACATTTTAAATAGGGGCATTGAAATTTACTCCCACCACCA | |
| | | GGGCTTTTTTGGGGGATGCCTGGGCCTTTAAAACACTAGC | |
| | | CAAACTCTAATTAATTCTCAAATCACTGCCAGGAGTTCTT | |
| | | GCTCCTGGCTGCAGGCCCAGGCCCCAAGGTCTCCTTCTTG | |
| | | GGGTCACAAACAGCAGTAAGGAAGAGGAATATATAGCAAC | |
| | | TCAGGGCCTGGGAATTGTGGGGCAATCCGTTCTTAGGGAC | |
| | | TGGATACTTCTGGCTGGCTGAGTATAGTACTAGCTGCCTC | |
| | | CCCACCAGGTTCCGAGTAGTGTCTGAGACTCTGCTCTGCA | |
| | | GGGCCTAGGGTAGCGCTGGGAGTGTAGAAGTGGCCTGCCC | |
| | | TTAACTGTTTTCACTAAACAGCTTTTTCTAAGGGGAGAGC | |
| | | AAGGGGGAGAGATCTAGATTGGGTGAGGGGGACGGGGATG | |
| | | TCAGGGAGGCAAGTGTGTTGTGTTACTGTGTCAATAAACT | |
| | | GATTTAAAGTTGTGAAAAAAAAAAAAA | |
| PANK2 | NM_024960.4 | ATGCTGGGGGAGGGGCTGGCGGCCTCGACGGCAGCTGCGG | 27 |
| | | AACTAGGCCGAGGGACAAAGGCTAAGTTTTTCCATGGTTT | |
| | | GGACTGGATATCGGTGGAACTCTGGTCAAGCTGGTATATT | |
| | | TTGAACCCAAAGACATCACTGCTGAAGAAGAAGAGGAAGA | |
| | | AGTGGAAAGTCTTAAAAGCATTCGGAAGTACCTGACCTCC | |
| | | AATGTGGCTTATGGGTCTACAGGCATTCGGGACGTGCACC | |
| | | TCGAGCTGAAGGACCTGACTCTGTGTGGACGCAAAGGCAA | |
| | | TCTGCACTTTATACGCTTTCCCACTCATGACATGCCTGCT | |
| | | TTTATTCAAATGGGCAGAGATAAAAACTTCTCGAGTCTCC | |
| | | ACACTGTCTTTTGTGCCACTGGAGGTGGAGCGTACAAATT | |
| | | TGAGCAGGATTTTCTCACAATAGGTGATCTTCAGCTTTGC | |
| | | AAACTGGATGAACTAGATTGCTTGATCAAAGGAATTTTAT | |
| | | ACATTGACTCAGTCGGATTCAATGGACGGTCACAGTGCTA | |
| | | TTACTTTGAAAACCCTGCTGATTCTGAAAAGTGTCAGAAG | |
| | | TTACCATTTGATTTGAAAAATCCGTATCCTCTGCTTCTGG | |
| | | TGAACATTGGCTCAGGGGTTAGCATCTTAGCAGTATATTC | |
| | | CAAAGATAATTACAAACGGGTCACAGGTACTAGTCTTGGA | |
| | | GGAGGAACTTTTTTTGGTCTCTGCTGTCTTCTTACTGGCT | |
| | | GTACCACTTTTGAAGAAGCTCTTGAAATGGCATCTCGTGG | |
| | | AGATAGCACCAAAGTGGATAAACTAGTACGAGATATTTAT | |
| | | GGAGGGGACTATGAGAGGTTTGGACTGCCAGGCTGGGCTG | |
| | | TGGCTTCAAGCTTTGGAAACATGATGAGCAAGGAGAAGCG | |
| | | AGAGGCTGTCAGTAAAGAGGACCTGGCCAGAGCGACTTTG | |
| | | ATCACCATCACCAACAACATTGGCTCAATAGCAAGAATGT | |
| | | GTGCCCTTAATGAAAACATTAACCAGGTGGTATTTGTTGG | |
| | | AAATTTCTTGAGAATTAATACGATCGCCATGCGGCTTTTG | |
| | | GCATATGCTTTGGATTATTGGTCCAAGGGGCAGTTGAAAG | |
| | | CACTTTTTTCGGAACACGAGGGTTATTTTGGAGCTGTTGG | |
| | | AGCACTCCTTGAGCTGTTGAAGATCCCGTGATCATTACCT | |
| | | GGGGAGGGGTTCCTGAAACCTTCCACAATGGGATCTGTGG | |
| | | ACTTTCATTTTTTTAAGAGACTTACTCAATTTCATGACTG | |
| | | TACTACCTGAAACAAAGTGAGAAAGGACAGGTGTATTTTT | |
| | | CTAAGTCATCAAGATAAATCCTTAAGAATTCAGTCTAAAT | |
| | | TAGCAACCAGGAAGGAAAAATATATTAAAAACAACAAAAA | |
| | | AGTGGCACATGTCCAGGCAGTGTGAGGATTTGCTGTATAT | |
| | | AAGTTGCCTGCTTTGTATTTTTGAAATCTCTGCATCACTC | |
| | | ATTGGAAGTGCTTCTGAAGAGAGCTGCTCTGTGTTCAGTT | |
| | | GACTGGTTTTGTGTCCTGTTTGAACTTGCTGAATGTAAGG | |
| | | CAGGCTACTATGCGTTATAATCTAATCACAATTTGTCAAT | |
| | | ATGGTCTTGGCAATCATCTGTGCATTACTCTGGTTTGCAT | |
| | | TAAGCCTGTGTGTGAACTTACTGTAAAACATGTTTTATTT | |
| | | CAAGGTTCTGCAAAATTAATTGGGCAGGTTAATTGTGTAC | |
| | | CTGAAACTTAACAAGCAGTTTTTGGAAGGGCA | |
| PHF21A | NM_001101802.1 | GGTGAATGGGCTGGTGGTGCTCGCTGCTGCTGCTGAGAGG | 28 |
| | | AGGAGGAGGATGAAGAGTTGGGCTTGTTTGTCTCCTACAG | |
| | | TTTCTCTCCTGCTGCTCTGATTCCCCCCTCCCGATTCCGG | |
| | | CCCGGGGCCTGTGTGTGTCCCTCCTGGAGGAGGAGGAGGA | |
| | | TCCAGTTCCTCCCCCCAACCCCCTCCTCCCCACCCCCCCT | |
| | | TGCCTGGGGAAGAGGAGGAAAGAAACAGCCCAGAGAGAGA | |
| | | GAGAGAGAGAGAGTGAGTGAGAGAGAGAGGAGAGGAGAGG | |
| | | AGGAGGAGGAGGAGGGAGAAGGGAACAACCTACCATCTTA | |
| | | ACACACTAATATCTAAAAAGTGCGAGAGGCCCAGAGCAGC | |
| | | AGCAGAAGCAGCAGCAGCAGCTCCAGCTTCTTCCCTCCCT | |
| | | CCCCATGAAGAAGAGTTCCCTCCTCCTCCTCCTCCTGCTT | |
| | | CTCCTGCTCAGAGTTCCTGCCTCCAGCTGCCAGGGGGGAC | |
| | | AGCCAGCCAGCAGCAGGAGGGGGGCTAGAGAGCTGAAGGA | |
| | | GAGCCAGTTTCCCCAAAATTGCTGCAGTGAGAAGAGGAGT | |
| | | TTGTTACTTTAAACAGAGGCTGAAGAAACTATAGAATTAG | |
| | | CAGAGAAAGTGGAGAAGGTAGAGGATGGAGTTGCAGACTC | |
| | | TACAGGAGGCTCTTAAAGTGGAAATTCAGGTTCACCAGAA | |
| | | ACTGGTTGCTCAAATGAAGCAGGATCCACAGAATGCTGAC | |
| | | TTAAAGAAACAGCTTCATGAACTCCAAGCCAAAATCACAG | |
| | | CTTTGAGTGAGAAACAGAAAAGAGTAGTTGAACAGCTACG | |
| | | GAAGAACCTGATAGTAAAGCAAGAACAACCGGACAAGTTC | |
| | | CAAATACAGCCATTGCCACAATCTGAAAACAAACTACAAA | |
| | | CAGCACAGCAGCAACCACTACAGCAACTACAACAACAGCA | |
| | | GCAGTACCACCACCACCACGCCCAGCAGTCAGCTGCAGCC | |
| | | TCTCCCAACCTGACTGCTTCACAGAAGACTGTAACTACAG | |
| | | CTTCTATGATTACCACAAAGACACTACCTCTCGTCTTGAA | |
| | | AGCAGCAACTGCGACCATGCCTGCCTCTGTGGTGGGCCAG | |
| | | AGACCTACCATTGCTATGGTGACCGCCATCAACAGTCAGA | |
| | | AGGCTGTGCTCAGCACTGATGTGCAGAACACACCAGTCAA | |
| | | CCTCCAGACGTCTAGTAAGGTCACTGGGCCTGGGGCAGAG | |
| | | GCTGTCCAAATTGTGGCAAAAAACACAGTCACTCTGGTTC | |
| | | AGGCAACACCTCCTCAGCCCATCAAAGTACCACAGTTTAT | |
| | | CCCCCCTCCTAGACTCACTCCACGTCCAAACTTTCTTCCA | |
| | | CAGGTTCGACCCAAGCCTGTGGCCCAGAATAACATTCCTA | |
| | | TTGCCCCAGCACCACCTCCCATGCTCGCAGCTCCTCAGCT | |
| | | TATCCAGAGGCCCGTCATGCTGACCAAGTTCACCCCCACA | |
| | | ACCCTTCCCACATCCCAGAATTCCATCCACCCCGTCCGTG | |
| | | TCGTCAATGGGCAGACTGCAACCATAGCCAAAACGTTCCC | |
| | | CATGGCCCAGCTCACCAGCATTGTGATAGCTACTCCAGGG | |
| | | ACCAGACTCGCTGGACCTCAAACTGTACAGCTTAGCAAGC | |
| | | CAAGTCTTGAAAAACAGACAGTTAAATCTCACACAGAAAC | |
| | | AGATGAGAAACAAACAGAGAGCCGCACCATCACCCCACCT | |
| | | GCTGCACCCAAACCAAAACGGGAGGAGAACCCTCAGAAAC | |
| | | TTGCCTTCATGGTGTCTCTAGGGTTGGTAACACATGACCA | |
| | | TCTAGAAGAAATCCAAAGCAAGAGGCAAGAGCGAAAAAGA | |
| | | AGAACAACAGCAAATCCGGTCTACAGTGGAGCAGTCTTTG | |
| | | AGCCAGAGCGTAAGAAGAGTGCAGTGACATACCTAAACAG | |
| | | CACAATGCACCCTGGGACCCGGAAGAGAGGTCGTCCTCCA | |
| | | AAATACAATGCAGTGCTGGGGTTTGGAGCCCTTACCCCAA | |
| | | CATCCCCCCAATCCAGTCATCCTGACTCCCCTGAAAATGA | |
| | | AAAGACAGAGACCACATTCACTTTCCCTGCACCTGTTCAG | |
| | | CCTGTGTCCCTGCCCAGCCCCACCTCCACAGACGGTGATA | |
| | | TTCATGAGGATTTTTGCAGCGTTTGCAGAAAAAGTGGCCA | |
| | | GTTACTGATGTGCGACACATGTTCCCGTGTATATCATTTG | |
| | | GACTGCTTAGACCCCCCTCTGAAAACAATTCCCAAGGGCA | |
| | | TGTGGATCTGTCCCAGATGTCAGGACCAGATGCTGAAGAA | |
| | | GGAAGAAGCAATTCCATGGCCTGGAACTTTAGCAATTGTT | |
| | | CATTCCTATATTGCCTACAAAGCAGCAAAAGAAGAAGAGA | |
| | | AACAGAAGTTACTTAAATGGAGTTCAGATTTAAAACAAGA | |
| | | ACGAGAACAACTAGAGCAAAAGGTGAAACAGCTCAGCAAT | |
| | | TCCATAAGTAAATGCATGGAAATGAAGAACACCATCCTGG | |
| | | CCCGGCAGAAGGAGATGCACAGCTCCCTGGAGAAGGTAAA | |
| | | ACAGCTGATTCGCCTCATCCACGGCATCGACCTCTCCAAA | |
| | | CCTGTAGACTCTGAGGCCACTGTGGGGGCCATCTCCAATG | |
| | | GCCCGGACTGCACCCCCCCTGCCAATGCCGCCACCTCCAC | |
| | | GCCGGCCCCTTCCCCCTCCTCCCAGAGCTGCACAGCGAAC | |
| | | TGTAACCAGGGGGAAGAGACTAAATAACAGAGCCCCTCTA | |
| | | GGAGAAGCCACGGGATCCCGGCGGCAAGGAGAACAGAACA | |
| | | CTGAAGACTCTAGAAAAGCAAAGCCGGATTTCTGGAAAGT | |
| | | GCAGAATTCTTTTGGTTCTTTGGTTCCAGAGAGAGAGAAG | |
| | | ATGCTTGTGCCAGGTGGCACCAGAGTTTGCCAATTGATCC | |
| | | TTCTTATTCTGTGTGTACATGCAAAGATTGGACCATGTTA | |
| | | CATGAAATAGTGCCAGCTGGAGGTTCTTTGCCAGCACCAT | |
| | | GCCAAGTGAAATAATATATTTACTCTCTCTATTATACACC | |
| | | AGTGTGTGCCTGCAGCAGCCTCCACAGCCACGATGGGTTT | |
| | | GTTTCTGTTTTCTTGGGTGGGGAGCAGGGACGGGCGGAGG | |
| | | GAGGAGAGCAGGTTTCAGATCCTTACTTGCCGAGCCGTTT | |
| | | GTTTAGGTAGAGAAGACAAGTCCAAAGAGTGTGTGGGCTT | |
| | | TCCTGTTTCTAAACTTTCGCTACTATAAAACCAAAAAAAG | |
| | | GAATTGAGATTTCACCAACCCCAGTGCCCAGAAGAGGGAA | |
| | | GGGGAGTGGCTGGAGGGAGCAGGGGGTGGGACAGTGTATC | |
| | | AAATAAGCAGTATTTAATCACCTCTGGCGGGGGCCTCGTG | |
| | | CAAGGGGAGACTGACACCAAGAACAGCCAGTAGGTTCTTC | |
| | | TCCCCTGCACTCTGCTCCCTGCGCGGTAACCCCACCACTC | |
| | | CTGAAGCCTGCCCAGTCTCCTTCCTTCCCTGCTTGGTGAG | |
| | | TCGCGCATCTCCGTGGTTATCCCGCTGTCTCCTCTCCAAG | |
| | | AACAAGCAGAGCCCGGGCCACTGGCCCTTGCCCAAGGCAG | |
| | | GGAAGAAGGATGTGTGTGTCCAGGAAGGAAAAAAAGGTGG | |
| | | ATCAGTGATTTTACTTGAAAACAAGCTCCATCCCTTTTCT | |
| | | ATATTTATAAGAAGAGAAGATCTTGAGTGAAGCAGCACGC | |
| | | GACCCAGGTGTGTGTGAATTGAATGGAGACGTTTCTTTTC | |
| | | TCTTTCTTTAATTTTTGTTTTTGTTCTTTTTTTCTTTAAG | |
| | | GAAAGTTTTATTTTACTGTTCATTTTACTTTCTTGGTAAC | |
| | | AAAAACTAAAATAAGGAATAGAAAAGCTGTTTTTCAGGCT | |
| | | GACAGTCCAATTAAGGGTAGCCAAGACCTTGCATGGTAGA | |
| | | GTAGGAATCATAGTGTCAGTGAGGTCCCGTGAGTCTTTGT | |
| | | GAGTCCTTGTGTCATCGTTCGGGCACTGTTTTTTTATGCA | |
| | | AGGGCAAAAATCTTTGTATCTGGGGAAAAAAAACTTTTTT | |
| | | TTAAATTAAAAAGGAAAATAAAAGATATTGAGGTCTTCCT | |
| | | AGTGTTACTTAAATTAAGATCAAGGTAAGAAACATTGTAA | |
| | | AAAAAAATTACAAAAGTGCTATTTGTTTCCTAAAAACAGT | |
| | | GATTTCTATTAAAAAGGTGTCAGAACTGGAGAAAATGCCG | |
| | | TGTAGTTATAATTTTTTAGCACAGACCCTGCTGATCACGA | |
| | | TGACATTTTGCCGTGTGTGTGTCTCTAGACTGGTGGGCCA | |
| | | GTCTCCTTGAAGGACAGAGGCGGAGCTCCCCACCCTTCTC | |
| | | TCTCCTCAGAAAAGACCGTGCTCTCTTCTTGGTGCAGGGA | |
| | | TCTTGTCTCCTGTTGTGAAGCCCAAATGGAAGCGTGGATG | |
| | | GTATCAGGGCCCTACCCGTGGTCTTCTCAGATTCTGCTAG | |
| | | AGCAAAAGGCTGGTGCCTAAATAAGATCCCTTCCTTTGGT | |
| | | GCTGCTTTTGGTCTTTCAGCCACCAGCATTATGAGTGCCT | |
| | | GGGGGACACCTCCGAGGGAACTGGCCAGCGGAGCTCTGTG | |
| | | GTGCGCACGCACCCTGGCCGTGACAGGAGGGTGCGGGAGT | |
| | | ACAGGCTGGCTGCATCAGCCCTTGGTGCTTAGAACAGAGG | |
| | | AGGAGTGACATGTTTTGAGGGTACGTCTCTGAGACAGAGC | |
| | | CCCAGCGTGGCCTTCGCTCTGTCTTGCCTTTGGGGAGAGG | |
| | | TCTGAAGCTCCCACTCCTTTCTCTGCCTGTTGGCTCCAGG | |
| | | CACCAGAAATTTACTCCACTCCACCCACCCACAAGCCTCC | |
| | | TGGGTGACCCTGGGCTAGAATTGCTGCGCTTGCCTCGGCT | |
| | | TGGCCGGTTGTGGCCTCTCCTTGAGAAAACCAGGGTTGTG | |
| | | AAAGACTCAGACCATTCTCTCATCTTGCCTTGTCAGAAGT | |
| | | AAATTGTGTCAGATTTGTGCTCTCGCTGGAGACCTTTGCC | |
| | | CCTTGCGTGCCCCTGGCCGATGGGAGGGCGGTGGAGGCTC | |
| | | TGTACCCTGGCCCTGCTGGAGCATCTCCCCCAAGCCCACT | |
| | | CCAGGCCCTGGGAATGGCCAGAGTCTAGGAGAGGTAGAAA | |
| | | CGATCCTATCAGCTTCTCTCCCACCCAATTAGGCCCAGAG | |
| | | AGACAAAGACAGATCTGAAAGCAAATGCAACAGAGAAGAG | |
| | | ACACTTCTTAGAGTAAAATGTGTCTCATCTCTATCAGCCA | |
| | | TCGCCTTTCATCTTCCCAGGGGCCTCAGAAGAAGGAATTA | |
| | | AGTTAGGCTGAACAGGCCTCAGAGTTAGGCCCTGGCTGCT | |
| | | TGATTGGCTGAGGGGGAAAGAGTTCCCTTTTCTCATTCAG | |
| | | AAACCAAGGTGCTGTGTCTAGTCAGGGAGCCTTGGAGATG | |
| | | CCTGGACTAGTTGGAGGAATCGTTGGCAGAGGATCAGAGA | |
| | | CCAGCAGCAGGCTGTCTGCCCTGTCTAGAGCTCTTCCCCT | |
| | | CAACTTGTCTGGGCCCATCTGGGGGTTGCCACACAACACC | |
| | | TAACTTACCTTTTCCTGAAAGAAGTTGGGAAACCATCATC | |
| | | ACTAGAGGCCTTTGCTCAGAGAGGAGCTGCCTTAGGAGTC | |
| | | TTGGGTCGGAGGACGGGGCTAGGAATTGACCAGGGCTTTG | |
| | | CCTGCCGCCCTCAGCAGTGTCGGGTACATTCTGACCTCGC | |
| | | CTGCAGCTGGGCTGTGGATTCTTCCTGACATTCAGATGTG | |
| | | AGCTGTTTTGGGAGTCAGCTAGTATGGAGTACGAGATGCA | |
| | | ACCCAGCCCCCAAACCTACATTCTGCACTCAAATTCCAAA | |
| | | ACACTGCTTTACTGTAAAGAAGAGGCCCCTGGCACCCAAT | |
| | | CTCCCTGTCCTTCACTGTCCCCTCAGACCTGGGCGGGGAG | |
| | | GGGGGGGGGCCTGTGACCACCTGAGACATACGCTCGTGAC | |
| | | ACTGCCCCACCCCAGCCACCTCCACTTGCTTCCTCCTCCT | |
| | | TCCCTCCGCTGCTCTTTCCCCACGGCCCAGAATTTAGCTG | |
| | | CTCTGACAGCCACTTTTGAGACCAGCTGGCTTTGTAGTCA | |
| | | CTTCAGAGAGCTGGAGCGGCTGCCCACTGGGCCCTGACTG | |
| | | GGAGTCCCCTGCCAGCTCCTGATCAGGCGCTGCGCCCTGG | |
| | | TGGCAGTGATGACTGGGAGTCCCCTGCCAGCTCCTGTCCA | |
| | | GGCGCTGCATCCTGGTAACAGTGAGGCCATGTTGCTGTCA | |
| | | TCTCCACCTCTGCATTCTTGCTGCCTGTGGGTCCTTTTTC | |
| | | TTTCATGGAGCCTGCTGGGTCTTGTCTCACCTGTGCTGAG | |
| | | CTCCTCTGGGGTTTTGATTTCTTCCTTCCTTATCAGGCCC | |
| | | TTTGGGGTAAGCCTGCTGGTTGTACCTGACATAGGGAGGC | |
| | | AGTTAGGGGCAGTCCCTGGTGGGGCCGCCCTGGCAGCCTC | |
| | | CAGCTGGCACCATCGTGTGCCTGGTTTCCCTGCAACACCT | |
| | | GCCTCTCTGTCCCTGCTGCTGCTTGGCTCAGGCCCAACAG | |
| | | GCAGCGTGCATGGAGGTGGTTACACACAGCTGTTTCCGTG | |
| | | AGGGTGACCGTGTCTGCAGCACGCTTCCGTCTCCGCATGC | |
| | | ACGGCTGCCTCTCCAGCCACCTCTGATACTTCTCTCTTGG | |
| | | GGCCATCAGAGCCTCCCTTGGGCTGTCACCTCCCAGCTCA | |
| | | CACACACTCTTCAGTGGTTTCCTCTCTTCATTCTCTTATA | |
| | | GGGCGTGGTCCTTCTTATTTATCTAAAGGGCTGAATTTAG | |
| | | GAGACTTTTTACCCAGGGGCAAAAGGCTCTTAGGGTAATG | |
| | | AGATGGATGGTGGCCCAGGTGCATTTTCCAGGGCCTGGGT | |
| | | TCTCCAGATCCCGTGGCTTCTGTTGAGTGGAGGCAACTTT | |
| | | GCTCTGTGTGAACCTCGCCCCTGTCCCTCTGCCGGGCACC | |
| | | CCTGGCAGGAAGCAGGACTCCCATCCTCACCCTGACTTAG | |
| | | ACTGTCCTCTGAGTCAGCTCCTCTCCAAGACAGGAGTGGG | |
| | | CAGCCCTGGGCAGTCTTCTGGCCCCTTGCTAAAGTGAGGG | |
| | | GCAGGAAGCTGGGGCTGCCCTCCAGAAAGCCGGGGTAGGA | |
| | | ACTCTGAAAAATACCTCCTCTAAACGGAAGCAGGGCTCTC | |
| | | CAGTTCCACTTGGCGCCCCCTCCCACAAGGCCCTTCCTCC | |
| | | CTGAGGACCCCACCCCCCTACCCCTTCCCCAGCAGCCTTT | |
| | | GGACCCTCACCTCTCTCCGGTGTCCGTGGGTCCTCAGCCC | |
| | | AGGGTGAGCTGCAGTCAGGCGGGATGGGACGGGCAGGCCA | |
| | | GAGGTCAGCCAGCTCCTAGCAGAGAAGAGCCAGCCAGACC | |
| | | CCAACCCTGTCTCTTGTCCATGCCCTTTGTGATTTCAGTC | |
| | | TTGGTAGACTTGTATTTGGAGTTTTGTGCTTCAAAGTTTT | |
| | | TGTTTTTGTTTGTTTGGTTTTTGTTTTGAGGGGGTGGGGG | |
| | | GGGATACAGAGCAGCTGATCAATTTGTATTTATTTATTTT | |
| | | AACATTTTACTAAATAAAGCCAAATAAAGCCTCTCAAAAA | |
| | | AAAAAAAAAAAA | |
| PKD1 | NM_000296.3 | GCACTGCAGCGCCAGCGTCCGAGCGGGCGGCCGAGCTCCC | 29 |
| | | GGAGCGGCCTGGCCCCGAGCCCCGAGCGGGCGTCGCTCAG | |
| | | CAGCAGGTCGCGGCCGCAGCCCCATCCAGCCCCGCGCCCG | |
| | | CCATGCCGTCCGCGGGCCCCGCCTGAGCTGCGGCCTCCGC | |
| | | GCGCGGGCGGGCCTGGGGACGGCGGGGCCATGCGCGCGCT | |
| | | GCCCTAACGATGCCGCCCGCCGCGCCCGCCCGCCTGGCGC | |
| | | TGGCCCTGGGCCTGGGCCTGTGGCTCGGGGCGCTGGCGGG | |
| | | GGGCCCCGGGCGCGGCTGCGGGCCCTGCGAGCCCCCCTGC | |
| | | CTCTGCGGCCCAGCGCCCGGCGCCGCCTGCCGCGTCAACT | |
| | | GCTCGGGCCGCGGGCTGCGGACGCTCGGTCCCGCGCTGCG | |
| | | CATCCCCGCGGACGCCACAGCGCTAGACGTCTCCCACAAC | |
| | | CTGCTCCGGGCGCTGGACGTTGGGCTCCTGGCGAACCTCT | |
| | | CGGCGCTGGCAGAGCTGGATATAAGCAACAACAAGATTTC | |
| | | TACGTTAGAAGAAGGAATATTTGCTAATTTATTTAATTTA | |
| | | AGTGAAATAAACCTGAGTGGGAACCCGTTTGAGTGTGACT | |
| | | GTGGCCTGGCGTGGCTGCCGCGATGGGCGGAGGAGCAGCA | |
| | | GGTGCGGGTGGTGCAGCCCGAGGCAGCCACGTGTGCTGGG | |
| | | CCTGGCTCCCTGGCTGGCCAGCCTCTGCTTGGCATCCCCT | |
| | | TGCTGGACAGTGGCTGTGGTGAGGAGTATGTCGCCTGCCT | |
| | | CCCTGACAACAGCTCAGGCACCGTGGCAGCAGTGTCCTTT | |
| | | TCAGCTGCCCACGAAGGCCTGCTTCAGCCAGAGGCCTGCA | |
| | | GCGCCTTCTGCTTCTCCACCGGCCAGGGCCTCGCAGCCCT | |
| | | CTCGGAGCAGGGCTGGTGCCTGTGTGGGGCGGCCCAGCCC | |
| | | TCCAGTGCCTCCTTTGCCTGCCTGTCCCTCTGCTCCGGCC | |
| | | CCCCGCCACCTCCTGCCCCCACCTGTAGGGGCCCCACCCT | |
| | | CCTCCAGCACGTCTTCCCTGCCTCCCCAGGGGCCACCCTG | |
| | | GTGGGGCCCCACGGACCTCTGGCCTCTGGCCAGCTAGCAG | |
| | | CCTTCCACATCGCTGCCCCGCTCCCTGTCACTGCCACACG | |
| | | CTGGGACTTCGGAGACGGCTCCGCCGAGGTGGATGCCGCT | |
| | | GGGCCGGCTGCCTCGCATCGCTATGTGCTGCCTGGGCGCT | |
| | | ATCACGTGACGGCCGTGCTGGCCCTGGGGGCCGGCTCAGC | |
| | | CCTGCTGGGGACAGACGTGCAGGTGGAAGCGGCACCTGCC | |
| | | GCCCTGGAGCTCGTGTGCCCGTCCTCGGTGCAGAGTGACG | |
| | | AGAGCCTTGACCTCAGCATCCAGAACCGCGGTGGTTCAGG | |
| | | CCTGGAGGCCGCCTACAGCATCGTGGCCCTGGGCGAGGAG | |
| | | CCGGCCCGAGCGGTGCACCCGCTCTGCCCCTCGGACACGG | |
| | | AGATCTTCCCTGGCAACGGGCACTGCTACCGCCTGGTGGT | |
| | | GGAGAAGGCGGCCTGGCTGCAGGCGCAGGAGCAGTGTCAG | |
| | | GCCTGGGCCGGGGCCGCCCTGGCAATGGTGGACAGTCCCG | |
| | | CCGTGCAGCGCTTCCTGGTCTCCCGGGTCACCAGGAGCCT | |
| | | AGACGTGTGGATCGGCTTCTCGACTGTGCAGGGGGTGGAG | |
| | | GTGGGCCCAGCGCCGCAGGGCGAGGCCTTCAGCCTGGAGA | |
| | | GCTGCCAGAACTGGCTGCCCGGGGAGCCACACCCAGCCAC | |
| | | AGCCGAGCACTGCGTCCGGCTCGGGCCCACCGGGTGGTGT | |
| | | AACACCGACCTGTGCTCAGCGCCGCACAGCTACGTCTGCG | |
| | | AGCTGCAGCCCGGAGGCCCAGTGCAGGATGCCGAGAACCT | |
| | | CCTCGTGGGAGCGCCCAGTGGGGACCTGCAGGGACCCCTG | |
| | | ACGCCTCTGGCACAGCAGGACGGCCTCTCAGCCCCGCACG | |
| | | AGCCCGTGGAGGTCATGGTATTCCCGGGCCTGCGTCTGAG | |
| | | CCGTGAAGCCTTCCTCACCACGGCCGAATTTGGGACCCAG | |
| | | GAGCTCCGGCGGCCCGCCCAGCTGCGGCTGCAGGTGTACC | |
| | | GGCTCCTCAGCACAGCAGGGACCCCGGAGAACGGCAGCGA | |
| | | GCCTGAGAGCAGGTCCCCGGACAACAGGACCCAGCTGGCC | |
| | | CCCGCGTGCATGCCAGGGGGACGCTGGTGCCCTGGAGCCA | |
| | | ACATCTGCTTGCCGCTGGACGCCTCCTGCCACCCCCAGGC | |
| | | CTGCGCCAATGGCTGCACGTCAGGGCCAGGGCTACCCGGG | |
| | | GCCCCCTATGCGCTATGGAGAGAGTTCCTCTTCTCCGTTC | |
| | | CCGCGGGGCCCCCCGCGCAGTACTCGGTCACCCTCCACGG | |
| | | CCAGGATGTCCTCATGCTCCCTGGTGACCTCGTTGGCTTG | |
| | | CAGCACGACGCTGGCCCTGGCGCCCTCCTGCACTGCTCGC | |
| | | CGGCTCCCGGCCACCCTGGTCCCCAGGCCCCGTACCTCTC | |
| | | CGCCAACGCCTCGTCATGGCTGCCCCACTTGCCAGCCCAG | |
| | | CTGGAGGGCACTTGGGCCTGCCCTGCCTGTGCCCTGCGGC | |
| | | TGCTTGCAGCCACGGAACAGCTCACCGTGCTGCTGGGCTT | |
| | | GAGGCCCAACCCTGGACTGCGGCTGCCTGGGCGCTATGAG | |
| | | GTCCGGGCAGAGGTGGGCAATGGCGTGTCCAGGCACAACC | |
| | | TCTCCTGCAGCTTTGACGTGGTCTCCCCAGTGGCTGGGCT | |
| | | GCGGGTCATCTACCCTGCCCCCCGCGACGGCCGCCTCTAC | |
| | | GTGCCCACCAACGGCTCAGCCTTGGTGCTCCAGGTGGACT | |
| | | CTGGTGCCAACGCCACGGCCACGGCTCGCTGGCCTGGGGG | |
| | | CAGTGTCAGCGCCCGCTTTGAGAATGTCTGCCCTGCCCTG | |
| | | GTGGCCACCTTCGTGCCCGGCTGCCCCTGGGAGACCAACG | |
| | | ATACCCTGTTCTCAGTGGTAGCACTGCCGTGGCTCAGTGA | |
| | | GGGGGAGCACGTGGTGGACGTGGTGGTGGAAAACAGCGCC | |
| | | AGCCGGGCCAACCTCAGCCTGCGGGTGACGGCGGAGGAGC | |
| | | CCATCTGTGGCCTCCGCGCCACGCCCAGCCCCGAGGCCCG | |
| | | TGTACTGCAGGGAGTCCTAGTGAGGTACAGCCCCGTGGTG | |
| | | GAGGCCGGCTCGGACATGGTCTTCCGGTGGACCATCAACG | |
| | | ACAAGCAGTCCCTGACCTTCCAGAACGTGGTCTTCAATGT | |
| | | CATTTATCAGAGCGCGGCGGTCTTCAAGCTCTCACTGACG | |
| | | GCCTCCAACCACGTGAGCAACGTCACCGTGAACTACAACG | |
| | | TAACCGTGGAGCGGATGAACAGGATGCAGGGTCTGCAGGT | |
| | | CTCCACAGTGCCGGCCGTGCTGTCCCCCAATGCCACGCTA | |
| | | GCACTGACGGCGGGCGTGCTGGTGGACTCGGCCGTGGAGG | |
| | | TGGCCTTCCTGTGGACCTTTGGGGATGGGGAGCAGGCCCT | |
| | | CCACCAGTTCCAGCCTCCGTACAACGAGTCCTTCCCGGTT | |
| | | CCAGACCCCTCGGTGGCCCAGGTGCTGGTGGAGCACAATG | |
| | | TCATGCACACCTACGCTGCCCCAGGTGAGTACCTCCTGAC | |
| | | CGTGCTGGCATCTAATGCCTTCGAGAACCTGACGCAGCAG | |
| | | GTGCCTGTGAGCGTGCGCGCCTCCCTGCCCTCCGTGGCTG | |
| | | TGGGTGTGAGTGACGGCGTCCTGGTGGCCGGCCGGCCCGT | |
| | | CACCTTCTACCCGCACCCGCTGCCCTCGCCTGGGGGTGTT | |
| | | CTTTACACGTGGGACTTCGGGGACGGCTCCCCTGTCCTGA | |
| | | CCCAGAGCCAGCCGGCTGCCAACCACACCTATGCCTCGAG | |
| | | GGGCACCTACCACGTGCGCCTGGAGGTCAACAACACGGTG | |
| | | AGCGGTGCGGCGGCCCAGGCGGATGTGCGCGTCTTTGAGG | |
| | | AGCTCCGCGGACTCAGCGTGGACATGAGCCTGGCCGTGGA | |
| | | GCAGGGCGCCCCCGTGGTGGTCAGCGCCGCGGTGCAGACG | |
| | | GGCGACAACATCACGTGGACCTTCGACATGGGGGACGGCA | |
| | | CCGTGCTGTCGGGCCCGGAGGCAACAGTGGAGCATGTGTA | |
| | | CCTGCGGGCACAGAACTGCACAGTGACCGTGGGTGCGGCC | |
| | | AGCCCCGCCGGCCACCTGGCCCGGAGCCTGCACGTGCTGG | |
| | | TCTTCGTCCTGGAGGTGCTGCGCGTTGAACCCGCCGCCTG | |
| | | CATCCCCACGCAGCCTGACGCGCGGCTCACGGCCTACGTC | |
| | | ACCGGGAACCCGGCCCACTACCTCTTCGACTGGACCTTCG | |
| | | GGGATGGCTCCTCCAACACGACCGTGCGGGGGTGCCCGAC | |
| | | GGTGACACACAACTTCACGCGGAGCGGCACGTTCCCCCTG | |
| | | GCGCTGGTGCTGTCCAGCCGCGTGAACAGGGCGCATTACT | |
| | | TCACCAGCATCTGCGTGGAGCCAGAGGTGGGCAACGTCAC | |
| | | CCTGCAGCCAGAGAGGCAGTTTGTGCAGCTCGGGGACGAG | |
| | | GCCTGGCTGGTGGCATGTGCCTGGCCCCCGTTCCCCTACC | |
| | | GCTACACCTGGGACTTTGGCACCGAGGAAGCCGCCCCCAC | |
| | | CCGTGCCAGGGGCCCTGAGGTGACGTTCATCTACCGAGAC | |
| | | CCAGGCTCCTATCTTGTGACAGTCACCGCGTCCAACAACA | |
| | | TCTCTGCTGCCAATGACTCAGCCCTGGTGGAGGTGCAGGA | |
| | | GCCCGTGCTGGTCACCAGCATCAAGGTCAATGGCTCCCTT | |
| | | GGGCTGGAGCTGCAGCAGCCGTACCTGTTCTCTGCTGTGG | |
| | | GCCGTGGGCGCCCCGCCAGCTACCTGTGGGATCTGGGGGA | |
| | | CGGTGGGTGGCTCGAGGGTCCGGAGGTCACCCACGCTTAC | |
| | | AACAGCACAGGTGACTTCACCGTTAGGGTGGCCGGCTGGA | |
| | | ATGAGGTGAGCCGCAGCGAGGCCTGGCTCAATGTGACGGT | |
| | | GAAGCGGCGCGTGCGGGGGCTCGTCGTCAATGCAAGCCGC | |
| | | ACGGTGGTGCCCCTGAATGGGAGCGTGAGCTTCAGCACGT | |
| | | CGCTGGAGGCCGGCAGTGATGTGCGCTATTCCTGGGTGCT | |
| | | CTGTGACCGCTGCACGCCCATCCCTGGGGGTCCTACCATC | |
| | | TCTTACACCTTCCGCTCCGTGGGCACCTTCAATATCATCG | |
| | | TCACGGCTGAGAACGAGGTGGGCTCCGCCCAGGACAGCAT | |
| | | CTTCGTCTATGTCCTGCAGCTCATAGAGGGGCTGCAGGTG | |
| | | GTGGGCGGTGGCCGCTACTTCCCCACCAACCACACGGTAC | |
| | | AGCTGCAGGCCGTGGTTAGGGATGGCACCAACGTCTCCTA | |
| | | CAGCTGGACTGCCTGGAGGGACAGGGGCCCGGCCCTGGCC | |
| | | GGCAGCGGCAAAGGCTTCTCGCTCACCGTGCTCGAGGCCG | |
| | | GCACCTACCATGTGCAGCTGCGGGCCACCAACATGCTGGG | |
| | | CAGCGCCTGGGCCGACTGCACCATGGACTTCGTGGAGCCT | |
| | | GTGGGGTGGCTGATGGTGGCCGCCTCCCCGAACCCAGCTG | |
| | | CCGTCAACACAAGCGTCACCCTCAGTGCCGAGCTGGCTGG | |
| | | TGGCAGTGGTGTCGTATACACTTGGTCCTTGGAGGAGGGG | |
| | | CTGAGCTGGGAGACCTCCGAGCCATTTACCACCCATAGCT | |
| | | TCCCCACACCCGGCCTGCACTTGGTCACCATGACGGCAGG | |
| | | GAACCCGCTGGGCTCAGCCAACGCCACCGTGGAAGTGGAT | |
| | | GTGCAGGTGCCTGTGAGTGGCCTCAGCATCAGGGCCAGCG | |
| | | AGCCCGGAGGCAGCTTCGTGGCGGCCGGGTCCTCTGTGCC | |
| | | CTTTTGGGGGCAGCTGGCCACGGGCACCAATGTGAGCTGG | |
| | | TGCTGGGCTGTGCCCGGCGGCAGCAGCAAGCGTGGCCCTC | |
| | | ATGTCACCATGGTCTTCCCGGATGCTGGCACCTTCTCCAT | |
| | | CCGGCTCAATGCCTCCAACGCAGTCAGCTGGGTCTCAGCC | |
| | | ACGTACAACCTCACGGCGGAGGAGCCCATCGTGGGCCTGG | |
| | | TGCTGTGGGCCAGCAGCAAGGTGGTGGCGCCCGGGCAGCT | |
| | | GGTCCATTTTCAGATCCTGCTGGCTGCCGGCTCAGCTGTC | |
| | | ACCTTCCGCCTGCAGGTCGGCGGGGCCAACCCCGAGGTGC | |
| | | TCCCCGGGCCCCGTTTCTCCCACAGCTTCCCCCGCGTCGG | |
| | | AGACCACGTGGTGAGCGTGCGGGGCAAAAACCACGTGAGC | |
| | | TGGGCCCAGGCGCAGGTGCGCATCGTGGTGCTGGAGGCCG | |
| | | TGAGTGGGCTGCAGGTGCCCAACTGCTGCGAGCCTGGCAT | |
| | | CGCCACGGGCACTGAGAGGAACTTCACAGCCCGCGTGCAG | |
| | | CGCGGCTCTCGGGTCGCCTACGCCTGGTACTTCTCGCTGC | |
| | | AGAAGGTCCAGGGCGACTCGCTGGTCATCCTGTCGGGCCG | |
| | | CGACGTCACCTACACGCCCGTGGCCGCGGGGCTGTTGGAG | |
| | | ATCCAGGTGCGCGCCTTCAACGCCCTGGGCAGTGAGAACC | |
| | | GCACGCTGGTGCTGGAGGTTCAGGACGCCGTCCAGTATGT | |
| | | GGCCCTGCAGAGCGGCCCCTGCTTCACCAACCGCTCGGCG | |
| | | CAGTTTGAGGCCGCCACCAGCCCCAGCCCCCGGCGTGTGG | |
| | | CCTACCACTGGGACTTTGGGGATGGGTCGCCAGGGCAGGA | |
| | | CACAGATGAGCCCAGGGCCGAGCACTCCTACCTGAGGCCT | |
| | | GGGGACTACCGCGTGCAGGTGAACGCCTCCAACCTGGTGA | |
| | | GCTTCTTCGTGGCGCAGGCCACGGTGACCGTCCAGGTGCT | |
| | | GGCCTGCCGGGAGCCGGAGGTGGACGTGGTCCTGCCCCTG | |
| | | CAGGTGCTGATGCGGCGATCACAGCGCAACTACTTGGAGG | |
| | | CCCACGTTGACCTGCGCGACTGCGTCACCTACCAGACTGA | |
| | | GTACCGCTGGGAGGTGTATCGCACCGCCAGCTGCCAGCGG | |
| | | CCGGGGCGCCCAGCGCGTGTGGCCCTGCCCGGCGTGGACG | |
| | | TGAGCCGGCCTCGGCTGGTGCTGCCGCGGCTGGCGCTGCC | |
| | | TGTGGGGCACTACTGCTTTGTGTTTGTCGTGTCATTTGGG | |
| | | GACACGCCACTGACACAGAGCATCCAGGCCAATGTGACGG | |
| | | TGGCCCCCGAGCGCCTGGTGCCCATCATTGAGGGTGGCTC | |
| | | ATACCGCGTGTGGTCAGACACACGGGACCTGGTGCTGGAT | |
| | | GGGAGCGAGTCCTACGACCCCAACCTGGAGGACGGCGACC | |
| | | AGACGCCGCTCAGTTTCCACTGGGCCTGTGTGGCTTCGAC | |
| | | ACAGAGGGAGGCTGGCGGGTGTGCGCTGAACTTTGGGCCC | |
| | | CGCGGGAGCAGCACGGTCACCATTCCACGGGAGCGGCTGG | |
| | | CGGCTGGCGTGGAGTACACCTTCAGCCTGACCGTGTGGAA | |
| | | GGCCGGCCGCAAGGAGGAGGCCACCAACCAGACGGTGCTG | |
| | | ATCCGGAGTGGCCGGGTGCCCATTGTGTCCTTGGAGTGTG | |
| | | TGTCCTGCAAGGCACAGGCCGTGTACGAAGTGAGCCGCAG | |
| | | CTCCTACGTGTACTTGGAGGGCCGCTGCCTCAATTGCAGC | |
| | | AGCGGCTCCAAGCGAGGGCGGTGGGCTGCACGTACGTTCA | |
| | | GCAACAAGACGCTGGTGCTGGATGAGACCACCACATCCAC | |
| | | GGGCAGTGCAGGCATGCGACTGGTGCTGCGGCGGGGCGTG | |
| | | CTGCGGGACGGCGAGGGATACACCTTCACGCTCACGGTGC | |
| | | TGGGCCGCTCTGGCGAGGAGGAGGGCTGCGCCTCCATCCG | |
| | | CCTGTCCCCCAACCGCCCGCCGCTGGGGGGCTCTTGCCGC | |
| | | CTCTTCCCACTGGGCGCTGTGCACGCCCTCACCACCAAGG | |
| | | TGCACTTCGAATGCACGGGCTGGCATGACGCGGAGGATGC | |
| | | TGGCGCCCCGCTGGTGTACGCCCTGCTGCTGCGGCGCTGT | |
| | | CGCCAGGGCCACTGCGAGGAGTTCTGTGTCTACAAGGGCA | |
| | | GCCTCTCCAGCTACGGAGCCGTGCTGCCCCCGGGTTTCAG | |
| | | GCCACACTTCGAGGTGGGCCTGGCCGTGGTGGTGCAGGAC | |
| | | CAGCTGGGAGCCGCTGTGGTCGCCCTCAACAGGTCTTTGG | |
| | | CCATCACCCTCCCAGAGCCCAACGGCAGCGCAACGGGGCT | |
| | | CACAGTCTGGCTGCACGGGCTCACCGCTAGTGTGCTCCCA | |
| | | GGGCTGCTGCGGCAGGCCGATCCCCAGCACGTCATCGAGT | |
| | | ACTCGTTGGCCCTGGTCACCGTGCTGAACGAGTACGAGCG | |
| | | GGCCCTGGACGTGGCGGCAGAGCCCAAGCACGAGCGGCAG | |
| | | CACCGAGCCCAGATACGCAAGAACATCACGGAGACTCTGG | |
| | | TGTCCCTGAGGGTCCACACTGTGGATGACATCCAGCAGAT | |
| | | CGCTGCTGCGCTGGCCCAGTGCATGGGGCCCAGCAGGGAG | |
| | | CTCGTATGCCGCTCGTGCCTGAAGCAGACGCTGCACAAGC | |
| | | TGGAGGCCATGATGCTCATCCTGCAGGCAGAGACCACCGC | |
| | | GGGCACCGTGACGCCCACCGCCATCGGAGACAGCATCCTC | |
| | | AACATCACAGGAGACCTCATCCACCTGGCCAGCTCGGACG | |
| | | TGCGGGCACCACAGCCCTCAGAGCTGGGAGCCGAGTCACC | |
| | | ATCTCGGATGGTGGCGTCCCAGGCCTACAACCTGACCTCT | |
| | | GCCCTCATGCGCATCCTCATGCGCTCCCGCGTGCTCAACG | |
| | | AGGAGCCCCTGACGCTGGCGGGCGAGGAGATCGTGGCCCA | |
| | | GGGCAAGCGCTCGGACCCGCGGAGCCTGCTGTGCTATGGC | |
| | | GGCGCCCCAGGGCCTGGCTGCCACTTCTCCATCCCCGAGG | |
| | | CTTTCAGCGGGGCCCTGGCCAACCTCAGTGACGTGGTGCA | |
| | | GCTCATCTTTCTGGTGGACTCCAATCCCTTTCCCTTTGGC | |
| | | TATATCAGCAACTACACCGTCTCCACCAAGGTGGCCTCGA | |
| | | TGGCATTCCAGACACAGGCCGGCGCCCAGATCCCCATCGA | |
| | | GCGGCTGGCCTCAGAGCGCGCCATCACCGTGAAGGTGCCC | |
| | | AACAACTCGGACTGGGCTGCCCGGGGCCACCGCAGCTCCG | |
| | | CCAACTCCGCCAACTCCGTTGTGGTCCAGCCCCAGGCCTC | |
| | | CGTCGGTGCTGTGGTCACCCTGGACAGCAGCAACCCTGCG | |
| | | GCCGGGCTGCATCTGCAGCTCAACTATACGCTGCTGGACG | |
| | | GCCACTACCTGTCTGAGGAACCTGAGCCCTACCTGGCAGT | |
| | | CTACCTACACTCGGAGCCCCGGCCCAATGAGCACAACTGC | |
| | | TCGGCTAGCAGGAGGATCCGCCCAGAGTCACTCCAGGGTG | |
| | | CTGACCACCGGCCCTACACCTTCTTCATTTCCCCGGGGAG | |
| | | CAGAGACCCAGCGGGGAGTTACCATCTGAACCTCTCCAGC | |
| | | CACTTCCGCTGGTCGGCGCTGCAGGTGTCCGTGGGCCTGT | |
| | | ACACGTCCCTGTGCCAGTACTTCAGCGAGGAGGACATGGT | |
| | | GTGGCGGACAGAGGGGCTGCTGCCCCTGGAGGAGACCTCG | |
| | | CCCCGCCAGGCCGTCTGCCTCACCCGCCACCTCACCGCCT | |
| | | TCGGCGCCAGCCTCTTCGTGCCCCCAAGCCATGTCCGCTT | |
| | | TGTGTTTCCTGAGCCGACAGCGGATGTAAACTACATCGTC | |
| | | ATGCTGACATGTGCTGTGTGCCTGGTGACCTACATGGTCA | |
| | | TGGCCGCCATCCTGCACAAGCTGGACCAGTTGGATGCCAG | |
| | | CCGGGGCCGCGCCATCCCTTTCTGTGGGCAGCGGGGCCGC | |
| | | TTCAAGTACGAGATCCTCGTCAAGACAGGCTGGGGCCGGG | |
| | | GCTCAGGTACCACGGCCCACGTGGGCATCATGCTGTATGG | |
| | | GGTGGACAGCCGGAGCGGCCACCGGCACCTGGACGGCGAC | |
| | | AGAGCCTTCCACCGCAACAGCCTGGACATCTTCCGGATCG | |
| | | CCACCCCGCACAGCCTGGGTAGCGTGTGGAAGATCCGAGT | |
| | | GTGGCACGACAACAAAGGGCTCAGCCCTGCCTGGTTCCTG | |
| | | CAGCACGTCATCGTCAGGGACCTGCAGACGGCACGCAGCG | |
| | | CCTTCTTCCTGGTCAATGACTGGCTTTCGGTGGAGACGGA | |
| | | GGCCAACGGGGGCCTGGTGGAGAAGGAGGTGCTGGCCGCG | |
| | | AGCGACGCAGCCCTTTTGCGCTTCCGGCGCCTGCTGGTGG | |
| | | CTGAGCTGCAGCGTGGCTTCTTTGACAAGCACATCTGGCT | |
| | | CTCCATATGGGACCGGCCGCCTCGTAGCCGTTTCACTCGC | |
| | | ATCCAGAGGGCCACCTGCTGCGTTCTCCTCATCTGCCTCT | |
| | | TCCTGGGCGCCAACGCCGTGTGGTACGGGGCTGTTGGCGA | |
| | | CTCTGCCTACAGCACGGGGCATGTGTCCAGGCTGAGCCCG | |
| | | CTGAGCGTCGACACAGTCGCTGTTGGCCTGGTGTCCAGCG | |
| | | TGGTTGTCTATCCCGTCTACCTGGCCATCCTTTTTCTCTT | |
| | | CCGGATGTCCCGGAGCAAGGTGGCTGGGAGCCCGAGCCCC | |
| | | ACACCTGCCGGGCAGCAGGTGCTGGACATCGACAGCTGCC | |
| | | TGGACTCGTCCGTGCTGGACAGCTCCTTCCTCACGTTCTC | |
| | | AGGCCTCCACGCTGAGGCCTTTGTTGGACAGATGAAGAGT | |
| | | GACTTGTTTCTGGATGATTCTAAGAGTCTGGTGTGCTGGC | |
| | | CCTCCGGCGAGGGAACGCTCAGTTGGCCGGACCTGCTCAG | |
| | | TGACCCGTCCATTGTGGGTAGCAATCTGCGGCAGCTGGCA | |
| | | CGGGGCCAGGCGGGCCATGGGCTGGGCCCAGAGGAGGACG | |
| | | GCTTCTCCCTGGCCAGCCCCTACTCGCCTGCCAAATCCTT | |
| | | CTCAGCATCAGATGAAGACCTGATCCAGCAGGTCCTTGCC | |
| | | GAGGGGGTCAGCAGCCCAGCCCCTACCCAAGACACCCACA | |
| | | TGGAAACGGACCTGCTCAGCAGCCTGTCCAGCACTCCTGG | |
| | | GGAGAAGACAGAGACGCTGGCGCTGCAGAGGCTGGGGGAG | |
| | | CTGGGGCCACCCAGCCCAGGCCTGAACTGGGAACAGCCCC | |
| | | AGGCAGCGAGGCTGTCCAGGACAGGACTGGTGGAGGGTCT | |
| | | GCGGAAGCGCCTGCTGCCGGCCTGGTGTGCCTCCCTGGCC | |
| | | CACGGGCTCAGCCTGCTCCTGGTGGCTGTGGCTGTGGCTG | |
| | | TCTCAGGGTGGGTGGGTGCGAGCTTCCCCCCGGGCGTGAG | |
| | | TGTTGCGTGGCTCCTGTCCAGCAGCGCCAGCTTCCTGGCC | |
| | | TCATTCCTCGGCTGGGAGCCACTGAAGGTCTTGCTGGAAG | |
| | | CCCTGTACTTCTCACTGGTGGCCAAGCGGCTGCACCCGGA | |
| | | TGAAGATGACACCCTGGTAGAGAGCCCGGCTGTGACGCCT | |
| | | GTGAGCGCACGTGTGCCCCGCGTACGGCCACCCCACGGCT | |
| | | TTGCACTCTTCCTGGCCAAGGAAGAAGCCCGCAAGGTCAA | |
| | | GAGGCTACATGGCATGCTGCGGAGCCTCCTGGTGTACATG | |
| | | CTTTTTCTGCTGGTGACCCTGCTGGCCAGCTATGGGGATG | |
| | | CCTCATGCCATGGGCACGCCTACCGTCTGCAAAGCGCCAT | |
| | | CAAGCAGGAGCTGCACAGCCGGGCCTTCCTGGCCATCACG | |
| | | CGGTCTGAGGAGCTCTGGCCATGGATGGCCCACGTGCTGC | |
| | | TGCCCTACGTCCACGGGAACCAGTCCAGCCCAGAGCTGGG | |
| | | GCCCCCACGGCTGCGGCAGGTGCGGCTGCAGGAAGCACTC | |
| | | TACCCAGACCCTCCCGGCCCCAGGGTCCACACGTGCTCGG | |
| | | CCGCAGGAGGCTTCAGCACCAGCGATTACGACGTTGGCTG | |
| | | GGAGAGTCCTCACAATGGCTCGGGGACGTGGGCCTATTCA | |
| | | GCGCCGGATCTGCTGGGGGCATGGTCCTGGGGCTCCTGTG | |
| | | CCGTGTATGACAGCGGGGGCTACGTGCAGGAGCTGGGCCT | |
| | | GAGCCTGGAGGAGAGCCGCGACCGGCTGCGCTTCCTGCAG | |
| | | CTGCACAACTGGCTGGACAACAGGAGCCGCGCTGTGTTCC | |
| | | TGGAGCTCACGCGCTACAGCCCGGCCGTGGGGCTGCACGC | |
| | | CGCCGTCACGCTGCGCCTCGAGTTCCCGGCGGCCGGCCGC | |
| | | GCCCTGGCCGCCCTCAGCGTCCGCCCCTTTGCGCTGCGCC | |
| | | GCCTCAGCGCGGGCCTCTCGCTGCCTCTGCTCACCTCGGT | |
| | | GTGCCTGCTGCTGTTCGCCGTGCACTTCGCCGTGGCCGAG | |
| | | GCCCGTACTTGGCACAGGGAAGGGCGCTGGCGCGTGCTGC | |
| | | GGCTCGGAGCCTGGGCGCGGTGGCTGCTGGTGGCGCTGAC | |
| | | GGCGGCCACGGCACTGGTACGCCTCGCCCAGCTGGGTGCC | |
| | | GCTGACCGCCAGTGGACCCGTTTCGTGCGCGGCCGCCCGC | |
| | | GCCGCTTCACTAGCTTCGACCAGGTGGCGCAGCTGAGCTC | |
| | | CGCAGCCCGTGGCCTGGCGGCCTCGCTGCTCTTCCTGCTT | |
| | | TTGGTCAAGGCTGCCCAGCAGCTACGCTTCGTGCGCCAGT | |
| | | GGTCCGTCTTTGGCAAGACATTATGCCGAGCTCTGCCAGA | |
| | | GCTCCTGGGGGTCACCTTGGGCCTGGTGGTGCTCGGGGTA | |
| | | GCCTACGCCCAGCTGGCCATCCTGCTCGTGTCTTCCTGTG | |
| | | TGGACTCCCTCTGGAGCGTGGCCCAGGCCCTGTTGGTGCT | |
| | | GTGCCCTGGGACTGGGCTCTCTACCCTGTGTCCTGCCGAG | |
| | | TCCTGGCACCTGTCACCCCTGCTGTGTGTGGGGCTCTGGG | |
| | | CACTGCGGCTGTGGGGCGCCCTACGGCTGGGGGCTGTTAT | |
| | | TCTCCGCTGGCGCTACCACGCCTTGCGTGGAGAGCTGTAC | |
| | | CGGCCGGCCTGGGAGCCCCAGGACTACGAGATGGTGGAGT | |
| | | TGTTCCTGCGCAGGCTGCGCCTCTGGATGGGCCTCAGCAA | |
| | | GGTCAAGGAGTTCCGCCACAAAGTCCGCTTTGAAGGGATG | |
| | | GAGCCGCTGCCCTCTCGCTCCTCCAGGGGCTCCAAGGTAT | |
| | | CCCCGGATGTGCCCCCACCCAGCGCTGGCTCCGATGCCTC | |
| | | GCACCCCTCCACCTCCTCCAGCCAGCTGGATGGGCTGAGC | |
| | | GTGAGCCTGGGCCGGCTGGGGACAAGGTGTGAGCCTGAGC | |
| | | CCTCCCGCCTCCAAGCCGTGTTCGAGGCCCTGCTCACCCA | |
| | | GTTTGACCGACTCAACCAGGCCACAGAGGACGTCTACCAG | |
| | | CTGGAGCAGCAGCTGCACAGCCTGCAAGGCCGCAGGAGCA | |
| | | GCCGGGCGCCCGCCGGATCTTCCCGTGGCCCATCCCCGGG | |
| | | CCTGCGGCCAGCACTGCCCAGCCGCCTTGCCCGGGCCAGT | |
| | | CGGGGTGTGGACCTGGCCACTGGCCCCAGCAGGACACCCC | |
| | | TTCGGGCCAAGAACAAGGTCCACCCCAGCAGCACTTAGTC | |
| | | CTCCTTCCTGGCGGGGGTGGGCCGTGGAGTCGGAGTGGAC | |
| | | ACCGCTCAGTATTACTTTCTGCCGCTGTCAAGGCCGAGGG | |
| | | CCAGGCAGAATGGCTGCACGTAGGTTCCCCAGAGAGCAGG | |
| | | CAGGGGCATCTGTCTGTCTGTGGGCTTCAGCACTTTAAAG | |
| | | AGGCTGTGTGGCCAACCAGGACCCAGGGTCCCCTCCCCAG | |
| | | CTCCCTTGGGAAGGACACAGCAGTATTGGACGGTTTCTAG | |
| | | CCTCTGAGATGCTAATTTATTTCCCCGAGTCCTCAGGTAC | |
| | | AGCGGGCTGTGCCCGGCCCCACCCCCTGGGCAGATGTCCC | |
| | | CCACTGCTAAGGCTGCTGGCTTCAGGGAGGGTTAGCCTGC | |
| | | ACCGCCGCCACCCTGCCCCTAAGTTATTACCTCTCCAGTT | |
| | | CCTACCGTACTCCCTGCACCGTCTCACTGTGTGTCTCGTG | |
| | | TCAGTAATTTATATGGTGTTAAAATGTGTATATTTTTGTA | |
| | | TGTCACTATTTTCACTAGGGCTGAGGGGCCTGCGCCCAGA | |
| | | GCTGGCCTCCCCCAACACCTGCTGCGCTTGGTAGGTGTGG | |
| | | TGGCGTTATGGCAGCCCGGCTGCTGCTTGGATGCGAGCTT | |
| | | GGCCTTGGGCCGGTGCTGGGGGCACAGCTGTCTGCCAGGC | |
| | | ACTCTCATCACCCCAGAGGCCTTGTCATCCTCCCTTGCCC | |
| | | CAGGCCAGGTAGCAAGAGAGCAGCGCCCAGGCCTGCTGGC | |
| | | ATCAGGTCTGGGCAAGTAGCAGGACTAGGCATGTCAGAGG | |
| | | ACCCCAGGGTGGTTAGAGGAAAAGACTCCTCCTGGGGGCT | |
| | | GGCTCCCAGGGTGGAGGAAGGTGACTGTGTGTGTGTGTGT | |
| | | GTGCGCGCGCGCACGCGCGAGTGTGCTGTATGGCCCAGGC | |
| | | AGCCTCAAGGCCCTCGGAGCTGGCTGTGCCTGCTTCTGTG | |
| | | TACCACTTCTGTGGGCATGGCCGCTTCTAGAGCCTCGACA | |
| | | CCCCCCCAACCCCCGCACCAAGCAGACAAAGTCAATAAAA | |
| | | GAGCTGTCTGACTGC | |
| PLD3 | NM_001031696.3 | GCATCCTCTCACCGCCGGAAGCTGAACTGACTCGTCCGCG | 30 |
| | | GCCGCTCTACCCCAACAGGCCGCCACCAGCGAGAGTGCGG | |
| | | CCATAACCATCACGTGACCGCCCACCGACACCAGCGAGAG | |
| | | TGCAGTCGTAACCGTCACGTGACCGCCCACCGTCGGCCCG | |
| | | GCGCTCCCCTCCGCCCGAAGCTAGCAAGCGGCGCGGCCAA | |
| | | TGAGAAAGGCGCATGCCTGGCCCCCGCCGGCCTGCAGTCT | |
| | | AGCCGTAGTGCGCCTGCGCGCGGCTAGGAGGGGCCGTCAG | |
| | | GCGGGGATACAGCCTGGAAGGTAATGCATGTCCATGGTAC | |
| | | ACAAATTCACAAGTTTGGAGACCCTGACACACCCACCTTC | |
| | | TCACCTGGGCTCTGCGTATCCCCCAGCCTTGAGGGAAGAT | |
| | | GAAGCCTAAACTGATGTACCAGGAGCTGAAGGTGCCTGCA | |
| | | GAGGAGCCCGCCAATGAGCTGCCCATGAATGAGATTGAGG | |
| | | CGTGGAAGGCTGCGGAAAAGAAAGCCCGCTGGGTCCTGCT | |
| | | GGTCCTCATTCTGGCGGTTGTGGGCTTCGGAGCCCTGATG | |
| | | ACTCAGCTGTTTCTATGGGAATACGGCGACTTGCATCTCT | |
| | | TTGGGCCCAACCAGCGCCCAGCCCCCTGCTATGACCCTTG | |
| | | CGAAGCAGTGCTGGTGGAAAGCATTCCTGAGGGCCTGGAC | |
| | | TTCCCCAATGCCTCCACGGGGAACCCTTCCACCAGCCAGG | |
| | | CCTGGCTGGGCCTGCTCGCCGGTGCGCACAGCAGCCTGGA | |
| | | CATCGCCTCCTTCTACTGGACCCTCACCAACAATGACACC | |
| | | CACACGCAGGAGCCCTCTGCCCAGCAGGGTGAGGAGGTCC | |
| | | TCCGGCAGCTGCAGACCCTGGCACCAAAGGGCGTGAACGT | |
| | | CCGCATCGCTGTGAGCAAGCCCAGCGGGCCCCAGCCACAG | |
| | | GCGGACCTGCAGGCTCTGCTGCAGAGCGGTGCCCAGGTCC | |
| | | GCATGGTGGACATGCAGAAGCTGACCCATGGCGTCCTGCA | |
| | | TACCAAGTTCTGGGTGGTGGACCAGACCCACTTCTACCTG | |
| | | GGCAGTGCCAACATGGACTGGCGTTCACTGACCCAGGTCA | |
| | | AGGAGCTGGGCGTGGTCATGTACAACTGCAGCTGCCTGGC | |
| | | TCGAGACCTGACCAAGATCTTTGAGGCCTACTGGTTCCTG | |
| | | GGCCAGGCAGGCAGCTCCATCCCATCAACTTGGCCCCGGT | |
| | | TCTATGACACCCGCTACAACCAAGAGACACCAATGGAGAT | |
| | | CTGCCTCAATGGAACCCCTGCTCTGGCCTACCTGGCGAGT | |
| | | GCGCCCCCACCCCTGTGTCCAAGTGGCCGCACTCCAGACC | |
| | | TGAAGGCTCTACTCAACGTGGTGGACAATGCCCGGAGTTT | |
| | | CATCTACGTCGCTGTCATGAACTACCTGCCCACTCTGGAG | |
| | | TTCTCCCACCCTCACAGGTTCTGGCCTGCCATTGACGATG | |
| | | GGCTGCGGCGGGCCACCTACGAGCGTGGCGTCAAGGTGCG | |
| | | CCTGCTCATCAGCTGCTGGGGACACTCGGAGCCATCCATG | |
| | | CGGGCCTTCCTGCTCTCTCTGGCTGCCCTGCGTGACAACC | |
| | | ATACCCACTCTGACATCCAGGTGAAACTCTTTGTGGTCCC | |
| | | CGCGGATGAGGCCCAGGCTCGAATCCCATATGCCCGTGTC | |
| | | AACCACAACAAGTACATGGTGACTGAACGCGCCACCTACA | |
| | | TCGGAACCTCCAACTGGTCTGGCAACTACTTCACGGAGAC | |
| | | GGCGGGCACCTCGCTGCTGGTGACGCAGAATGGGAGGGGC | |
| | | GGCCTGCGGAGCCAGCTGGAGGCCATTTTCCTGAGGGACT | |
| | | GGGACTCCCCTTACAGCCATGACCTTGACACCTCAGCTGA | |
| | | CAGCGTGGGCAACGCCTGCCGCCTGCTCTGAGGCCCGATC | |
| | | CAGTGGGCAGGCCAAGGCCTGCTGGGCCCCCGCGGACCCA | |
| | | GGTGCTCTGGGTCACGGTCCCTGTCCCCGCGCCCCCGCTT | |
| | | CTGTCTGCCCCATTGTGGCTCCTCAGGCTCTCTCCCCTGC | |
| | | TCTCCCACCTCTACCTCCACCCCCACCGGCCTGACGCTGT | |
| | | GGCCCCGGGACCCAGCAGAGCTGGGGGAGGGATCAGCCCC | |
| | | CAAAGAAATGGGGGTGCATGCTGGGCCTGGCCCCCTGGCC | |
| | | CACCCCCACTTTCCAGGGCAAAAAGGGCCCAGGGTTATAA | |
| | | TAAGTAAATAACTTGTCTGTACAGCCTGAAAAAAAAAAAA | |
| | | AAAAAAA | |
| PNMA2 | NM_007257.5 | GAGCGGTGCTCAGGGGAGGGCTGGAGGGGAGGGAAGGAGA | 31 |
| | | GAGAGAGGGGAGGGCGGCACCGCCCCTAGCCCCGCGCTCC | |
| | | GGAAGTGAAGCGGCCAGACCACCAGCTAATGGATGCGGAG | |
| | | CGGAGGGCCCGCTGACCGCTCTCCGCGCCTGGAGCAGCTT | |
| | | GGCTTGGCTGGAGCTAAGAGCCAGACACACCACTGTGTGG | |
| | | AGGTGGGTGATGTCTTCCTGTGCTAAAAGGTGAATAAATA | |
| | | AGCTCCTCACCTCTCGCGGAACACTCGGGAACACATCAAC | |
| | | AGGGGTCCAAGCCGCCCTGCTGGGAGGCTTCTCTTCAAGA | |
| | | GTTCTGGGTCCCAGAGTGGAAGGCATTTTCCCATCAACTG | |
| | | GAGAGAGACGAAACATCAGAGACCAGGAGGCTGTGGAGAA | |
| | | AGCAGCTGTCCCAGGTGCCTCAACTATCAGAGAAGGGTCA | |
| | | GCGTCACGTGGCTGCCAGCATCTTTGAGAAAATCACTGGC | |
| | | AATCGGACTTCAGAGCTGCGGGCACAGGTGTGGTTAGAAC | |
| | | TGAGATACGACCTGCCCACCTGGGTCAGGCCTAAAGACAA | |
| | | GAAGTCCTGAGTTCTTGCCACTGAGTAGGCCAGGGTCATT | |
| | | TGTCCAGAAAACTTTGTGACTGTCTTTGAGTGACCTAGTC | |
| | | TGGGACCCATTCATTGGTGGGTTCTAAGGTTAGAAGCTCA | |
| | | TCCAGGATATTTTCAATATTAAGTCAGTGCATAGCTGCAC | |
| | | CACTAACAAATTGGTGCCTGTAGAGTCAGAGTGGGTCAAT | |
| | | TCTTAGGACAATGGCGCTGGCACTGTTAGAGGACTGGTGC | |
| | | AGGATAATGAGTGTGGATGAGCAGAAGTCACTGATGGTTA | |
| | | CGGGGATACCGGCGGACTTTGAGGAGGCTGAGATTCAGGA | |
| | | GGTCCTTCAGGAGACTTTAAAGTCTCTGGGCAGGTATAGA | |
| | | CTGCTTGGCAAGATATTCCGGAAGCAGGAGAATGCCAATG | |
| | | CTGTCTTACTAGAGCTTCTGGAAGATACTGATGTCTCGGC | |
| | | CATTCCCAGTGAGGTCCAGGGAAAGGGGGGTGTCTGGAAG | |
| | | GTGATCTTTAAGACCCCTAATCAGGACACTGAGTTTCTTG | |
| | | AAAGATTGAACCTGTTTCTAGAAAAAGAGGGGCAGACGGT | |
| | | CTCGGGTATGTTTCGAGCCCTGGGGCAGGAGGGCGTGTCT | |
| | | CCAGCCACAGTGCCCTGCATCTCACCAGAATTACTGGCCC | |
| | | ATTTGTTGGGACAGGCAATGGCACATGCGCCTCAGCCCCT | |
| | | GCTACCCATGAGATACCGGAAACTGCGAGTATTCTCAGGG | |
| | | AGTGCTGTCCCAGCCCCAGAGGAAGAGTCCTTTGAGGTCT | |
| | | GGTTGGAACAGGCCACGGAGATAGTCAAAGAGTGGCCAGT | |
| | | AACAGAGGCAGAAAAGAAAAGGTGGCTGGCGGAAAGCCTG | |
| | | CGGGGCCCTGCCCTGGACCTCATGCACATAGTGCAGGCAG | |
| | | ACAACCCGTCCATCAGTGTAGAAGAGTGTTTGGAGGCCTT | |
| | | TAAGCAAGTGTTTGGGAGCCTAGAGAGCCGCAGGACAGCC | |
| | | CAGGTGAGGTATCTGAAGACCTATCAGGAGGAAGGAGAGA | |
| | | AGGTCTCAGCCTATGTGTTACGGCTAGAAACCCTGCTCCG | |
| | | GAGAGCGGTGGAGAAACGCGCCATCCCTCGGCGTATTGCG | |
| | | GACCAGGTCCGCCTGGAGCAGGTCATGGCTGGGGCCACTC | |
| | | TTAACCAGATGCTGTGGTGCCGGCTTAGGGAGCTGAAGGA | |
| | | TCAGGGCCCGCCCCCCAGCTTCCTTGAGCTAATGAAGGTA | |
| | | ATACGGGAAGAAGAGGAGGAAGAGGCCTCCTTTGAGAATG | |
| | | AGAGTATCGAAGAGCCAGAGGAACGAGATGGCTATGGCCG | |
| | | CTGGAATCATGAGGGAGACGACTGAAAACCACCTGGGGGC | |
| | | AGGACCCACAGCCAGTGGGCTAAGACCTTTAAAAAATTTT | |
| | | TTTCTTTAATGTATGGGACTGAAATCAAACCATGAAAGCC | |
| | | AATTATTGACCTTCCTTCCTTCCTTCCTTCCCTCCCTTCC | |
| | | TCCTTCTCTCCTTCTCTCCTCCTCTCTCCTCTCCTCTCCT | |
| | | CTCTTTCCTTCCTTCCTTCCTTTTTTCTTTTTCTCTTTCT | |
| | | TCTTTATTTCTTGGGTCTCACTCTCATCACCCAGGCTAGA | |
| | | GTGCAGTGGCACAAAAATCTCGGCTCACTGCAGCCTTGAC | |
| | | TTCCCAGGCTCAGGCTCAGGTGATCCTCACACCTTAGCCT | |
| | | CCCAAGTACCTGGGACTACAGGCACGCACCACCATGCCTA | |
| | | GCTATTCTTTTGTATTTTTGGTAGAGACAGGGTTTTGCTG | |
| | | TGTTGCTCAGGCTGGTCTGGAACCCCTAGGCTCAAATGAT | |
| | | GTGCCCAACTCGGCCTCCCAAAGTGCTGGGATTACAGGCA | |
| | | TGAACCGCCATGCCTGGCCCTTGATTTTTCTTTTTAAGAA | |
| | | AAAAATATCTAGGAGTTTCTTAGACCCTATGTAGATTATT | |
| | | AATGAACAAAAGATTAAACTCCAAATATTAAATAGTAAGC | |
| | | CTGAAGGAATCTGAAACACTTGTACTTCCAATTTTCTTTA | |
| | | AATAATCCCAAATAGACCAGAATTGGCCCATACCATAGAA | |
| | | GAAAGAATTGGCAGTCAAAAAAAAAAATACCTTTTGTAAT | |
| | | GTTTGAAAAATAAAGCTGTTTGACTTGTCAGGTGTTTTCC | |
| | | TTTCTCAAATCAGCAAATTCTCTCTGAGTGCCTGGCTTTG | |
| | | TGAGACACTGTACAAGGAGTTACAAGACTACAGCTATAAC | |
| | | CTGCAGTTGAGCAGTTATAAACCTACAAAATGGGCCCTGC | |
| | | CCTCAGAGAGGTTCCAGTCTAGATGAGGAGCTGATCTAGA | |
| | | CAGGTAAAAGGCTAACTAACCCTTTGTGTAAATAAGTTCA | |
| | | TCACCCCAGTAAAAGTGTCATCACCCAGTGAATAGGACCA | |
| | | CCTCTGCCTGCAGATTTTTGTTGTTGTTGTTGTCATTGTT | |
| | | GTTGTTGTTTTAACCTGGGAAGTGTTCTTCCTGCCTTTCT | |
| | | GCTAGGTGTCAGATAGATGGTCCCAGAGCTAGGTGCTGTG | |
| | | TCAGGCCCTGAAGACACAGATGACTCAACCTAAGCTTTAC | |
| | | TTTCCAGAGGTCCACAGCCTGAGAGGTGTCCCCAAAGAAA | |
| | | GGGGGACATGAGGGGACTGCATGCTTGAGAGCAGGGTTGT | |
| | | TTAGGGCAGGTTTGGATTTAGTGAGCAGGCTGGTTTGCTT | |
| | | AGAGAAGGCTTTTAGTGGCAACAAAGGATGAAGAGGAGAG | |
| | | AAAAGGAACTCACATTTATTGAGGGCCTACTGTGTGCAAA | |
| | | GTGTTTCATGTATATCTCATTGAATGTATACAGCCACCCT | |
| | | GTTGTGGTATAATTTTGCTCTTTATAAAGAGAAAGACCGA | |
| | | AGCTCAGATGAGTTAAGTGGTCTCCTCAACACCAAAATGC | |
| | | CAAGAAGTGATGGAGCCTAGACAGAAGCCCAGAACTTTCT | |
| | | GACTCACACTAGTCCATCCTCTACCATCACGATGACTTTC | |
| | | AAATTGTGCTCTGCAGTTCTGCAGATTTTCTAGCAGTGCC | |
| | | ATCTCCAAAATGTGTTTTAAACTCTTTATTTTTTTAATTA | |
| | | TTATTAGTATTATTTTGAGACTGAGTCTTGCTCTATCACC | |
| | | CAGGCTGGAGTGCAGTGGTGCAATCTCAGCTCACTGCAAC | |
| | | CTCCGCCTCCCAGGTTCAAGCGATTTCGTGCCTCAGCCTC | |
| | | CCGAGTAGCTGGGATTACAGGCACCCACCACCACGCCCAG | |
| | | CTAATTTTTGTATTTTTAGTAGAAATGGGGTTTCACCATG | |
| | | TTGGCCAGGCTGGTCTCGAACTCCTGACCTCAAGTGATCC | |
| | | ACTCACCTCGGCCTCCCAAAGTGCTGGGATTACAGGTGTG | |
| | | AGCCACCATGCCTGGGCTAAACTCTTTAAGTCTCTAGTAA | |
| | | ATGCAGCTAGATTCAAATGGGCTGATAACCAAATTTTAAC | |
| | | ACATCAGCATTCACCACCAGGTTTACTTTTATTTTCAGAT | |
| | | TGGCTCATTTTGTGCAGACCTTAGAGCAAAGTTTCCTTTA | |
| | | TGGTATCTGTGTACGTATCCAAACTTCTTTTAATTGTTCA | |
| | | CAGATTTTAAAAGCGGTAGCACCACATGGTTGTGTAGATC | |
| | | AGACCTGTGTATTTAGATCAGACCTGTGTATCACGTAAGT | |
| | | GTGTGAGTGCAGTGCAGATGAGCACCATTTAGTTATATGT | |
| | | GCTAGGCAAATCTCCAACACAGTTGATGTGTAGTCTTGTG | |
| | | GTAGATTTGTGCATACTGTAAGCAAATTGCTTAGCTTCTC | |
| | | TAGACATCAGTTTCCACATCTGAAAAATAAGAAGATGAGA | |
| | | GTACACGGTTGTTATGAACAAATGACTTAATGCTTTTTAA | |
| | | GCACGTTGCATGACATCTGGAACACAGAAAGCCCTCAATA | |
| | | CATTGAAGCTCTTAGGATTTTCACGATGTTCCTGTCTGCT | |
| | | CAATGCATGCTTTCTTTATTGTTCTGACAGTTGTGTGGTA | |
| | | ACAAGCTAATATGCTTCCAGTTGACTTCCAGTCTACCCTG | |
| | | GTGTTAGAAACCGTTTCATCTCTTATTGTAAATTTGAGTG | |
| | | CTTGTTGTTTTTTATATTTGTGATGACTCTTCCAGCAGTT | |
| | | GTTGACAATTGTTAGAGGTTTGACTTTTAAATAATTACTT | |
| | | ATTTTTTCTGATTGTGGTTCAGTTTAACTGAAGAATATCC | |
| | | TGAGATTGTAAGAAAAGCATTTTTTAAAAGGTATCACTTG | |
| | | TGATCATTTATCTTTCTAAATTCTATTTTTAATACTGTTC | |
| | | CACCAAAGTGATGCAGTGGTTACCATGACACCCTAATTTC | |
| | | ATGTGTTTTTGTATTTATGAAAATAGTTTCATTGTCATTT | |
| | | ATTGGCGGTATACAAAGTAAAATGTTATAAATGTGAAGTT | |
| | | ATAAAATAAATATATGCTAATAAAATCCTGAGTTTTTCTG | |
| | | TTTCCT | |
| PQBP1 | NM_001032381.1 | TGCCTCCTGAGCGTAGTCCAGTTACTTTCAGGCTCGGGGA | 32 |
| | | GTGAAGGCCTCGTTGAGAGAAGGTCTCATTCGGTGTTTTG | |
| | | GGAAGAGAGTCGTGTGGGCCCAGGTCTGTCTGCTATCAGC | |
| | | TATGCCGCTGCCCGTTGCGCTGCAGACCCGCTTGGCCAAG | |
| | | AGAGGCATCCTCAAACATCTGGAGCCTGAACCAGAGGAAG | |
| | | AGATCATTGCCGAGGACTATGACGATGATCCTGTGGACTA | |
| | | CGAGGCCACCAGGTTGGAGGGCCTACCACCAAGCTGGTAC | |
| | | AAGGTGTTCGACCCTTCCTGCGGGCTCCCTTACTACTGGA | |
| | | ATGCAGACACAGACCTTGTATCCTGGCTCTCCCCACATGA | |
| | | CCCCAACTCCGTGGTTACCAAATCGGCCAAGAAGCTCAGA | |
| | | AGCAGTAATGCAGATGCTGAAGAAAAGTTGGACCGGAGCC | |
| | | ATGACAAGTCGGACAGGGGCCATGACAAGTCGGACCGCAG | |
| | | CCATGAGAAACTAGACAGGGGCCACGACAAGTCAGACCGG | |
| | | GGCCACGACAAGTCTGACAGGGATCGAGAGCGTGGCTATG | |
| | | ACAAGGTAGACAGAGAGAGAGAGCGAGACAGGGAACGGGA | |
| | | TCGGGACCGCGGGTATGACAAGGCAGACCGGGAAGAGGGC | |
| | | AAAGAACGGCGCCACCATCGCCGGGAGGAGCTGGCTCCCT | |
| | | ATCCCAAGAGCAAGAAGGCAGTAAGCCGAAAGGATGAAGA | |
| | | GTTAGACCCCATGGACCCTAGCTCATACTCAGACGCCCCC | |
| | | CGGGGCACGTGGTCAACAGGACTCCCCAAGCGGAATGAGG | |
| | | CCAAGACTGGCGCTGACACCACAGCAGCTGGGCCCCTCTT | |
| | | CCAGCAGCGGCCGTATCCATCCCCAGGGGCTGTGCTCCGG | |
| | | GCCAATGCAGAGGCCTCCCGAACCAAGCAGCAGGATTGAA | |
| | | GCTTCGGCCTCCCTGGCCCTGGGTTAAAATAAAAGCTTTC | |
| | | TGGTGATCCTGCCCACCAAAAAAAAAAAAAAAAAAAAAAA | |
| | | AAAAAAAAAAAAAA | |
| RAF1 | NM_002880.3 | AGAATCGGAGAGCCGGTGGCGTCGCAGGTCGGGAGGACGA | 33 |
| | | GCACCGAGTCGAGGGCTCGCTCGTCTGGGCCGCCCGAGAG | |
| | | TCTTAATCGCGGGCGCTTGGGCCGCCATCTTAGATGGCGG | |
| | | GAGTAAGAGGAAAACGATTGTGAGGCGGGAACGGCTTTCT | |
| | | GCTGCCTTTTTTGGGCCCCGAAAAGGGTCAGCTGGCCGGG | |
| | | CTTTGGGGCGCGTGCCCTGAGGCGCGGAGCGCGTTTGCTA | |
| | | CGATGCGGGGGCTGCTCGGGGCTCCGTCCCCTGGGCTGGG | |
| | | GACGCGCCGAATGTGACCGCCTCCCGCTCCCTCACCCGCC | |
| | | GCGGGGAGGAGGAGCGGGCGAGAAGCTGCCGCCGAACGAC | |
| | | AGGACGTTGGGGCGGCCTGGCTCCCTCAGGTTTAAGAATT | |
| | | GTTTAAGCTGCATCAATGGAGCACATACAGGGAGCTTGGA | |
| | | AGACGATCAGCAATGGTTTTGGATTCAAAGATGCCGTGTT | |
| | | TGATGGCTCCAGCTGCATCTCTCCTACAATAGTTCAGCAG | |
| | | TTTGGCTATCAGCGCCGGGCATCAGATGATGGCAAACTCA | |
| | | CAGATCCTTCTAAGACAAGCAACACTATCCGTGTTTTCTT | |
| | | GCCGAACAAGCAAAGAACAGTGGTCAATGTGCGAAATGGA | |
| | | ATGAGCTTGCATGACTGCCTTATGAAAGCACTCAAGGTGA | |
| | | GGGGCCTGCAACCAGAGTGCTGTGCAGTGTTCAGACTTCT | |
| | | CCACGAACACAAAGGTAAAAAAGCACGCTTAGATTGGAAT | |
| | | ACTGATGCTGCGTCTTTGATTGGAGAAGAACTTCAAGTAG | |
| | | ATTTCCTGGATCATGTTCCCCTCACAACACACAACTTTGC | |
| | | TCGGAAGACGTTCCTGAAGCTTGCCTTCTGTGACATCTGT | |
| | | CAGAAATTCCTGCTCAATGGATTTCGATGTCAGACTTGTG | |
| | | GCTACAAATTTCATGAGCACTGTAGCACCAAAGTACCTAC | |
| | | TATGTGTGTGGACTGGAGTAACATCAGACAACTCTTATTG | |
| | | TTTCCAAATTCCACTATTGGTGATAGTGGAGTCCCAGCAC | |
| | | TACCTTCTTTGACTATGCGTCGTATGCGAGAGTCTGTTTC | |
| | | CAGGATGCCTGTTAGTTCTCAGCACAGATATTCTACACCT | |
| | | CACGCCTTCACCTTTAACACCTCCAGTCCCTCATCTGAAG | |
| | | GTTCCCTCTCCCAGAGGCAGAGGTCGACATCCACACCTAA | |
| | | TGTCCACATGGTCAGCACCACCCTGCCTGTGGACAGCAGG | |
| | | ATGATTGAGGATGCAATTCGAAGTCACAGCGAATCAGCCT | |
| | | CACCTTCAGCCCTGTCCAGTAGCCCCAACAATCTGAGCCC | |
| | | AACAGGCTGGTCACAGCCGAAAACCCCCGTGCCAGCACAA | |
| | | AGAGAGCGGGCACCAGTATCTGGGACCCAGGAGAAAAACA | |
| | | AAATTAGGCCTCGTGGACAGAGAGATTCAAGCTATTATTG | |
| | | GGAAATAGAAGCCAGTGAAGTGATGCTGTCCACTCGGATT | |
| | | GGGTCAGGCTCTTTTGGAACTGTTTATAAGGGTAAATGGC | |
| | | ACGGAGATGTTGCAGTAAAGATCCTAAAGGTTGTCGACCC | |
| | | AACCCCAGAGCAATTCCAGGCCTTCAGGAATGAGGTGGCT | |
| | | GTTCTGCGCAAAACACGGCATGTGAACATTCTGCTTTTCA | |
| | | TGGGGTACATGACAAAGGACAACCTGGCAATTGTGACCCA | |
| | | GTGGTGCGAGGGCAGCAGCCTCTACAAACACCTGCATGTC | |
| | | CAGGAGACCAAGTTTCAGATGTTCCAGCTAATTGACATTG | |
| | | CCCGGCAGACGGCTCAGGGAATGGACTATTTGCATGCAAA | |
| | | GAACATCATCCATAGAGACATGAAATCCAACAATATATTT | |
| | | CTCCATGAAGGCTTAACAGTGAAAATTGGAGATTTTGGTT | |
| | | TGGCAACAGTAAAGTCACGCTGGAGTGGTTCTCAGCAGGT | |
| | | TGAACAACCTACTGGCTCTGTCCTCTGGATGGCCCCAGAG | |
| | | GTGATCCGAATGCAGGATAACAACCCATTCAGTTTCCAGT | |
| | | CGGATGTCTACTCCTATGGCATCGTATTGTATGAACTGAT | |
| | | GACGGGGGAGCTTCCTTATTCTCACATCAACAACCGAGAT | |
| | | CAGATCATCTTCATGGTGGGCCGAGGATATGCCTCCCCAG | |
| | | ATCTTAGTAAGCTATATAAGAACTGCCCCAAAGCAATGAA | |
| | | GAGGCTGGTAGCTGACTGTGTGAAGAAAGTAAAGGAAGAG | |
| | | AGGCCTCTTTTTCCCCAGATCCTGTCTTCCATTGAGCTGC | |
| | | TCCAACACTCTCTACCGAAGATCAACCGGAGCGCTTCCGA | |
| | | GCCATCCTTGCATCGGGCAGCCCACACTGAGGATATCAAT | |
| | | GCTTGCACGCTGACCACGTCCCCGAGGCTGCCTGTCTTCT | |
| | | AGTTGACTTTGCACCTGTCTTCAGGCTGCCAGGGGAGGAG | |
| | | GAGAAGCCAGCAGGCACCACTTTTCTGCTCCCTTTCTCCA | |
| | | GAGGCAGAACACATGTTTTCAGAGAAGCTGCTGCTAAGGA | |
| | | CCTTCTAGACTGCTCACAGGGCCTTAACTTCATGTTGCCT | |
| | | TCTTTTCTATCCCTTTGGGCCCTGGGAGAAGGAAGCCATT | |
| | | TGCAGTGCTGGTGTGTCCTGCTCCCTCCCCACATTCCCCA | |
| | | TGCTCAAGGCCCAGCCTTCTGTAGATGCGCAAGTGGATGT | |
| | | TGATGGTAGTACAAAAAGCAGGGGCCCAGCCCCAGCTGTT | |
| | | GGCTACATGAGTATTTAGAGGAAGTAAGGTAGCAGGCAGT | |
| | | CCAGCCCTGATGTGGAGACACATGGGATTTTGGAAATCAG | |
| | | CTTCTGGAGGAATGCATGTCACAGGCGGGACTTTCTTCAG | |
| | | AGAGTGGTGCAGCGCCAGACATTTTGCACATAAGGCACCA | |
| | | AACAGCCCAGGACTGCCGAGACTCTGGCCGCCCGAAGGAG | |
| | | CCTGCTTTGGTACTATGGAACTTTTCTTAGGGGACACGTC | |
| | | CTCCTTTCACAGCTTCTAAGGTGTCCAGTGCATTGGGATG | |
| | | GTTTTCCAGGCAAGGCACTCGGCCAATCCGCATCTCAGCC | |
| | | CTCTCAGGGAGCAGTCTTCCATCATGCTGAATTTTGTCTT | |
| | | CCAGGAGCTGCCCCTATGGGGCGGGGCCGCAGGGCCAGCC | |
| | | TTGTTTCTCTAACAAACAAACAAACAAACAGCCTTGTTTC | |
| | | TCTAGTCACATCATGTGTATACAAGGAAGCCAGGAATACA | |
| | | GGTTTTCTTGATGATTTGGGTTTTAATTTTGTTTTTATTG | |
| | | CACCTGACAAAATACAGTTATCTGATGGTCCCTCAATTAT | |
| | | GTTATTTTAATAAAATAAATTAAATTTAGGTGTAAAAAAA | |
| | | AAAAAAAAAAA | |
| RNF41 | NM_001242826.1 | GATGTCCCAGGGGTATTGGGGCGGGGGGTTGAAATAACTG | 34 |
| | | GGGTTCAGGAGGAGGGATGGTGGTAGAGATAAAAATGTGA | |
| | | GAAGGGAGCAGCACTGGCGAGGAGTCGGGAGAGTACTCCT | |
| | | GATTGTGACATCACATTCATCCCCTGGGCGATGGAGCTTG | |
| | | TCACTGGGAAGGAATACTCAGTCGGAGAATAGCCAACAAG | |
| | | ATGGGTTACTGGGAGAATCTCTTCAGTGGCACTGAGTGGA | |
| | | GGCATCAGGGGGTTGGAGCCTTGTGAACAGGGAACCTGCC | |
| | | CCCCAACACTTGGAAGGACCTGGGTTTCAGTGATGAGACA | |
| | | TGGGGTATGATGTAACCCGTTTCCAGGGGGATGTTGACGA | |
| | | AGATCTTATCTGCCCTATTTGCAGTGGAGTCTTGGAGGAG | |
| | | CCAGTACAGGCACCTCATTGTGAACATGCTTTCTGCAACG | |
| | | CCTGCATCACCCAGTGGTTCTCTCAGCAACAGACATGTCC | |
| | | AGTGGACCGTAGTGTTGTGACGGTCGCCCATCTGCGCCCA | |
| | | GTACCTCGGATCATGCGGAACATGTTGTCAAAGCTGCAGA | |
| | | TTGCCTGTGACAACGCTGTGTTCGGCTGTAGTGCCGTTGT | |
| | | CCGGCTTGACAACCTCATGTCTCACCTCAGCGACTGTGAG | |
| | | CACAACCCGAAGCGGCCTGTGACCTGTGAACAGGGCTGTG | |
| | | GCCTGGAGATGCCCAAAGATGAGCTGCCCAACCATAACTG | |
| | | CATTAAGCACCTGCGCTCAGTGGTACAGCAGCAGCAGACA | |
| | | CGCATCGCAGAGCTGGAGAAGACGTCAGCTGAACACAAAC | |
| | | ACCAGCTGGCGGAGCAGAAGCGAGACATCCAGCTGCTAAA | |
| | | GGCATACATGCGTGCAATCCGCAGTGTCAACCCCAACCTT | |
| | | CAGAACCTGGAGGAGACAATTGAATACAACGAGATCCTAG | |
| | | AGTGGGTGAACTCCCTTCAGCCAGCAAGAGTGACCCGCTG | |
| | | GGGAGGGATGATCTCGACTCCTGATGCTGTGCTCCAGGCT | |
| | | GTAATCAAGCGCTCCCTGGTGGAGAGTGGCTGTCCTGCTT | |
| | | CTATTGTCAACGAGCTGATTGAAAATGCCCACGAGCGTAG | |
| | | CTGGCCCCAGGGTCTGGCCACACTAGAGACTAGACAGATG | |
| | | AACCGACGCTACTATGAGAACTACGTGGCCAAGCGCATCC | |
| | | CTGGCAAGCAGGCTGTTGTCGTGATGGCCTGTGAGAACCA | |
| | | GCACATGGGGGATGACATGGTGCAAGAGCCAGGCCTTGTC | |
| | | ATGATATTTGCGCATGGCGTGGAAGAGATATAAGAGAACT | |
| | | CGACTGGCTATCAGGAAGAGATGGAAATCAGAAAATCCCA | |
| | | TCACTCCAGCAGCTGGGACCTGAGTCCTACCCACCATTCT | |
| | | TAATACTGTGGCTTATACCTGAGCCACACATCTCCCTGCC | |
| | | CTTCTGGCACTGAAGGGCCTTGGGGTAGTTTGCTCAGCCT | |
| | | TTCAGGTGGGAAACCCAGATTTCCTCCCTTTGCCATATTC | |
| | | CCCTAAAATGTCTATAAATTATCAGTCTGGGTGGGAAAGC | |
| | | CCCCACCTCCATCCATTTTCCTGCTTAGGGTCCCTGGTTC | |
| | | CAGTTATTTTCAGAAAGCACAAAGAGATTCAATTTCCCTG | |
| | | GAGGATCAGGACAGAGGAAGGAATCTCTAATCGTCCCTCT | |
| | | CCTCCAAAACCAGGGAATCAGAGCAGTCAGGCCTGTTGAC | |
| | | TCTAAGCAGCAGACATCCTGAAGAAATGGTAAGGGTGGAG | |
| | | CCAAATCTCTAGAAATAAGTAGTGAGGCCGTTAATTGGCC | |
| | | ATCACTGATGGCCCTTAGGGAAAGACTGGACCTCTGTGCC | |
| | | AAGCAGTATCCCTGTTCAGCCCACCTTAAAGGTGTAGGCA | |
| | | CCCACTGGGTCTACCAGTATGCAGGTTGGGATACTGAAAA | |
| | | TTTCCAGATGAGCTCTTCTTTCCTACAAGTTTTCATAATT | |
| | | AGGGAATGCCAGGGTTTAGGGTAGGGGTTAATCTGTTGGG | |
| | | GGTTGATGTGTTTAGCAAGAAGCTACTCCTAGCTTTTGCT | |
| | | AAAATATGGTTGGCACTGCCTCTTGTGGCACAGGCCATAA | |
| | | TTGTTCCATAGACCCCTCTCTAGCCCTGTGACTGTAGTTA | |
| | | GTTACTTTGATAATTTTCTTTGGCCATTGTTTGTTTATAT | |
| | | TTCACAAACTCCACCTACTGCCCCCCCCCCTCTTTTTTTT | |
| | | AAGAATGGCCTGATCATGGCTATCTCAGCCACATTGTTGG | |
| | | CAATTTAATTTATTTACTTCCTTTTTTTTTTTTTAAGAAA | |
| | | GGAAAAAAGAAAAAAAAATCAAACTTGAAACTTTTCTTTT | |
| | | GATGTTCCTATTGTGGGGGTTCTGGATAGGGTGGGACAGG | |
| | | GATGGGGGTGTGTTTTATATTTTTTCCTTTTCAGCACAAC | |
| | | CTTTGGCTTTAATATAGGAAGAGCCAAGGGAGTCCTCGGC | |
| | | TGAACTTACGATATCTGCCCCAAACCTCTGTAACCCCAAC | |
| | | TGAAATGAGGAGCTTCCTCTCTTCCTGTGAAGGATATGAC | |
| | | AGTCCAGCATCGATGCCTGTGCCCTCTGGAAAAATTTCCT | |
| | | CCTAGCCCTTCCAGGGCCTTATCATAAAACTCTGGATTTA | |
| | | GAGTATTCATTTTGAAGGCAACTCCCCCTTCCCCAAGTTT | |
| | | CCTTGGAGCTGTATAGCTGGGTTCTAAGCTTCACCATGCA | |
| | | AATCAGAAATTTTATCTCTAAGTACAGGCTGTGCCGTGTC | |
| | | TCACCCACACCCCCCTGGGGACTTCAGTTCCATTTCAGGT | |
| | | TACCTGGGGTATACCTTGATCCCTAGAGTGACTGGCAGAG | |
| | | TAAGAGAAGGGGAGAGATAATAGGTGTGATTATTTTAATA | |
| | | TGGAGGTGGGAGTGTGGTTGGAGATAGAAAGGCTCCTCCC | |
| | | CACCATGTAATGGCTTCCTCTCAGAATTTTATTCCAGGCT | |
| | | AGCTTGCTGCAGGTCTGGGTAGTTGGATCATGGCTCCACT | |
| | | GGGATTGGGGTGGAAAGCTTGAGGGGAGTAGGGTTCCAGC | |
| | | TCTGGGACATTGTGCTCAGGAATTTGAAAACGCTGCTATA | |
| | | CTTACTCTGGTTACTACATTTCTTCCACTCCCCTTTCCCC | |
| | | TACCTGCCTTAACCAAGGCTCATACTGTCCTGTCCTTACC | |
| | | CTCAGATGGAGCCAGGAAGCTCAGTGAAAGGCTTCCCTAC | |
| | | CCTTTGCACTAGTGTCTCTGCAGGTTGCTGGTTGTGTTGT | |
| | | ATGTGCTGTTCCATGGTGTTGACTGCACTAATAATAAACC | |
| | | TTTTACTCAACTCTCTAAATTCTTCAGCATTACTCCCTTT | |
| | | CTTGAGAAGGTTTCCCCTCTGCTTTTGCCTTTCTCTCACC | |
| | | TTAATTCCCTTTCTTCCTTACTTTGTTACCTACCCTTATC | |
| | | TTAGTGCTAACTTCTCTTTCAGGAGGATGTCTGGGAGTAG | |
| | | TGTGCACTTCACAGCTGCTTTCCCATGTACCCTCCTGCAT | |
| | | TCTTCCCTCCTATCTCCTGTTCTGTAGCAGCCAAAGCTCT | |
| | | CTAGTGATCTGAACTGTGTGCTTCCCAGGGTCTGCCTTTA | |
| | | TCCTAAATTCCATGTCTTCCCTGAGTGGTCCTGAGTTTTT | |
| | | GGGATAATTTCTACAGAAGATATGTATATATCTTTTTCCT | |
| | | TTGTCCCACAAGCAACTTTGCTTTAGAATCTAGAATTCCT | |
| | | TTGCAGGCAGAGAAGTCTCTACCTCCCAGTGTTTCCTAGC | |
| | | TAAGAACGTAAATGTGAGGAGGGAAATGTACTTGCAGAGG | |
| | | TTTCATAATTATTTACTTATAAAAATAGTCTTCATAGCCG | |
| | | GGCGCGGTGGCTCACGCCTGTAATCCCAGCACTTTGGGAG | |
| | | GCCGAGGTGGGTGGATCACAAGGTCAGGAGTTCGAGACCA | |
| | | TCCTGGCTAACACAGTGAAACCCCGTCTCTACTAAAAATA | |
| | | CAAAAAATTAGCCGGGCGTGGTGGCAGGCACCTGTAGTCC | |
| | | CAGCTACTTAGGAGGCTGAGGCAGGAGAATGGCGTGAACC | |
| | | CGGGAGGCAGAGCTTGCAGTGAGCAGAGATTGGGCCACTG | |
| | | CATTCCAGCCTGGGCGACAGAGCAAGGCTCCGTCTAAAAA | |
| | | AAAAAAAAAAAAAAAAAGTCTTCATAGGCCGGGCACGGTG | |
| | | GCTCACGTCTGTAATCCCAGCACTTTGGGAGGCCAAGGTG | |
| | | GGTGGATCACAACGTCAGGAGATCGAGACCATCCTGGCTA | |
| | | ACATGGTGAAACCCTGTCTCTACTAAAAATATAAATAAAT | |
| | | TAGCCGGACAGGCGCCTGTCCTCCCAGCTACTCAGGAGGC | |
| | | TGAGGCAGGAGAATGGTGTGAACCTGGGAGGCGGAGCTTG | |
| | | CAGTGAGCTGAGATCACGCCACTGCACTCCAGCCTGGGCA | |
| | | ACAGAGCAAGACTCCGTCTCAAAAAAAAAAAAAAAAAAAC | |
| | | CAGTCTTCATAAGTATTTGCTGCTACCTTTCCCTGTCATA | |
| | | AGAAAAAGGATAGCCAGACATGGTGGGACGCCACTATGAT | |
| | | CCCAGCTCCTTGGAAGGCTAAGGCACAAGAATCGCTTGAA | |
| | | CCTGGGAGGTGGAGGTTGCAGTGAGCTGAGATCATGCCAC | |
| | | TGCACTCCAGCCTGGTGACAGAGCAAGAGCCTGTCTCAAA | |
| | | AAAAAAAAAGAAAAGAAAAGAAAAAGGGATATCTTTTCCT | |
| | | CCTCCCAGAAGTTTGTTTTAAATTTGAGCATTTATCATGC | |
| | | ACCTGATGTAAACCTAATAGTACTCTTGATACTCTAGTGG | |
| | | CTTGAAAAAAAAAAAAAAGGCATTTCTGTGCTGAGTCTGC | |
| | | GCTTCTATGCACACAAGGTATGTTTATAAAATACTGATAA | |
| | | GCATGTCACAGTATAGAGCATAAGAGGCAATGTATGTATC | |
| | | CTAGTGACATTAGCAGTGCTTTTCCCCCCTTAAACTCCTT | |
| | | TAAAATTACTTTTAGAACTTGCTGCTCATTCTTGTGAATG | |
| | | TTATGAATGGTGTCATATTGTCCTTTTACAGAAGATACGA | |
| | | TTTTTAGAAACAAATATTCATTGAATGTCTGCCCTGTGAG | |
| | | ATACTCACTAGAGTGAACATGAGGAGGCTTATGTAGCAAA | |
| | | ATGGCACCTACCTGCAAAGAACTTAGTCCCTAATGGAGAT | |
| | | GAATATATAATAAGGGATCATAAATGTGCTAAGTGGATTT | |
| | | ACTAGTAATATGTGAGCCAAGGACGATAAAGCTCCTGATT | |
| | | CTGATGGGTATCAGGAAAGGCTTTTCAGGAAGTGTTACTT | |
| | | GTTATAGGTCAGAGGTCAGCAAACTACAGGTTACAACCCC | |
| | | ACTGCCTGCTTTTGTAAAAAACTTTATTGGAATACAGTTA | |
| | | TGCCCACTTGTTTATA | |
| RSF1 | NM_016578.3 | GATCCGCAGAGGAGCCCACTTGAGAGCGCCTCCTGTCGTC | 35 |
| | | TGTAAGGTTGCCTTGCCATCCCTCGGCACCCCAACTTCCC | |
| | | CCGCCCCCCCATCGCCTCCTCCTCCATCCTCCAGTTCAAA | |
| | | ATGGCGACGGCGGCGGCAGCGGCGGCGGTGATGGCTCCTC | |
| | | CGGGCTGCCCGGGTTCGTGCCCCAACTTCGCCGTAGTCTG | |
| | | CTCCTTCTTGGAGCGCTACGGGCCGCTGCTAGACCTGCCT | |
| | | GAGTTGCCGTTCCCTGAGCTGGAGCGGGTGCTGCAGGCGC | |
| | | CGCCGCCGGACGTCGGCAACGGAGAAGTACCAAAAGAATT | |
| | | GGTGGAGCTCCATTTGAAGCTGATGAGGAAAATTGGCAAA | |
| | | TCTGTTACTGCAGACAGATGGGAAAAATATTTGATCAAGA | |
| | | TATGCCAAGAGTTTAACAGTACCTGGGCATGGGAGATGGA | |
| | | GAAGAAGGGCTATCTTGAAATGAGTGTTGAATGCAAACTA | |
| | | GCACTCTTAAAGTACCTCTGTGAGTGTCAGTTTGATGACA | |
| | | ATCTCAAATTCAAGAATATTATTAATGAGGAGGATGCCGA | |
| | | TACTATGCGTCTCCAGCCAATTGGTCGAGACAAAGATGGC | |
| | | CTCATGTACTGGTACCAATTGGATCAAGATCACAATGTCA | |
| | | GAATGTACATAGAAGAACAAGATGATCAAGATGGCTCTTC | |
| | | ATGGAAATGCATTGTCAGAAATCGAAACGAGTTGGCTGAG | |
| | | ACTCTTGCACTCCTGAAAGCACAAATTGATCCTGTACTAT | |
| | | TGAAAAACTCTAGCCAACAAGACAACTCTTCTCGGGAAAG | |
| | | TCCCAGCTTAGAGGATGAGGAGACTAAAAAAGAGGAAGAA | |
| | | ACACCTAAACAAGAGGAACAGAAAGAAAGTGAAAAGATGA | |
| | | AAAGTGAGGAGCAGCCTATGGATTTAGAAAACCGTTCTAC | |
| | | AGCCAATGTTCTAGAAGAGACTACTGTGAAAAAAGAAAAA | |
| | | GAAGATGAAAAGGAACTTGTGAAACTGCCAGTCATAGTGA | |
| | | AGCTAGAAAAACCTTTGCCAGAAAATGAAGAAAAAAAGAT | |
| | | TATCAAAGAAGAAAGTGATTCCTTCAAGGAAAATGTCAAA | |
| | | CCCATTAAAGTTGAGGTGAAGGAATGTAGAGCAGATCCTA | |
| | | AAGATACCAAAAGTAGCATGGAGAAGCCAGTGGCACAGGA | |
| | | GCCTGAAAGGATCGAATTTGGTGGCAATATTAAATCTTCT | |
| | | CACGAAATTACTGAGAAATCTACTGAAGAAACTGAGAAAC | |
| | | TTAAAAATGACCAGCAGGCCAAGATACCACTAAAAAAACG | |
| | | AGAAATTAAACTGAGTGATGATTTTGACAGTCCAGTCAAG | |
| | | GGACCTTTGTGTAAATCAGTTACTCCAACAAAAGAGTTTT | |
| | | TGAAAGATGAAATAAAACAAGAGGAAGAGACTTGTAAAAG | |
| | | GATCTCTACAATCACTGCTTTGGGTCATGAAGGGAAACAG | |
| | | CTGGTAAATGGAGAAGTTAGTGATGAAAGGGTAGCTCCAA | |
| | | ATTTTAAGACAGAACCAATAGAGACAAAGTTTTATGAGAC | |
| | | AAAGGAAGAGAGCTATAGCCCCTCTAAGGACAGAAATATC | |
| | | ATCACGGAGGGAAATGGAACAGAGTCCTTAAATTCTGTCA | |
| | | TAACAAGTATGAAAACAGGTGAGCTTGAGAAAGAAACAGC | |
| | | CCCTTTGAGGAAAGATGCAGATAGTTCAATATCAGTCTTA | |
| | | GAGATCCATAGTCAAAAAGCACAAATAGAGGAACCCGATC | |
| | | CTCCAGAAATGGAAACTTCTCTTGATTCTTCTGAGATGGC | |
| | | AAAAGATCTCTCTTCAAAAACTGCTTTATCTTCCACCGAG | |
| | | TCGTGTACCATGAAAGGTGAAGAGAAGTCTCCCAAAACTA | |
| | | AGAAGGATAAGCGCCCACCAATCCTAGAATGTCTTGAAAA | |
| | | GTTAGAGAAGTCCAAAAAGACTTTTCTTGATAAGGACGCA | |
| | | CAAAGATTGAGTCCAATACCAGAAGAAGTTCCAAAGAGTA | |
| | | CTCTAGAGTCAGAAAAGCCTGGCTCTCCTGAGGCAGCTGA | |
| | | AACTTCTCCACCATCTAATATCATTGACCACTGTGAGAAA | |
| | | CTAGCCTCAGAAAAAGAAGTGGTAGAATGCCAGAGTACAA | |
| | | GTACTGTTGGTGGCCAGTCTGTGAAAAAAGTAGACCTAGA | |
| | | AACCCTAAAAGAGGATTCTGAGTTCACAAAGGTAGAAATG | |
| | | GATAATCTGGACAATGCCCAGACCTCTGGCATAGAGGAGC | |
| | | CTTCTGAGACAAAGGGTTCTATGCAAAAAAGCAAATTCAA | |
| | | ATATAAGTTGGTTCCTGAAGAAGAAACCACTGCCTCAGAA | |
| | | AATACAGAGATAACCTCTGAAAGGCAGAAAGAGGGCATCA | |
| | | AATTAACAATCAGGATATCAAGTCGGAAAAAGAAGCCCGA | |
| | | TTCTCCCCCCAAAGTTCTAGAACCAGAAAACAAGCAAGAG | |
| | | AAGACAGAAAAGGAAGAGGAGAAAACAAATGTGGGTCGTA | |
| | | CTTTAAGAAGATCTCCAAGAATATCTAGACCCACTGCAAA | |
| | | AGTGGCTGAGATCAGAGATCAGAAAGCTGATAAAAAAAGA | |
| | | GGGGAAGGAGAAGATGAGGTGGAAGAAGAGTCAACAGCTT | |
| | | TGCAAAAAACTGACAAAAAGGAAATTTTGAAAAAATCAGA | |
| | | GAAAGATACAAATTCTAAAGTAAGCAAGGTAAAACCCAAA | |
| | | GGCAAAGTTCGATGGACTGGTTCTCGGACACGTGGCAGAT | |
| | | GGAAATATTCCAGCAATGATGAAAGTGAAGGGTCTGGCAG | |
| | | TGAAAAATCATCTGCAGCTTCAGAAGAGGAGGAAGAAAAG | |
| | | GAAAGTGAAGAAGCCATCCTAGCAGATGATGATGAACCAT | |
| | | GCAAAAAATGTGGCCTTCCAAACCATCCTGAGCTAATTCT | |
| | | TCTGTGTGACTCTTGCGATAGTGGATACCATACTGCCTGC | |
| | | CTTCGCCCTCCTCTGATGATCATCCCAGATGGAGAATGGT | |
| | | TCTGCCCACCTTGCCAACATAAACTGCTCTGTGAAAAATT | |
| | | AGAGGAACAGTTGCAGGATTTGGATGTTGCCTTAAAGAAG | |
| | | AAAGAGCGTGCCGAACGAAGAAAAGAACGCTTGGTGTATG | |
| | | TTGGTATCAGTATTGAAAACATCATTCCTCCACAAGAGCC | |
| | | AGACTTTTCTGAAGATCAAGAAGAAAAGAAAAAAGATTCA | |
| | | AAAAAATCCAAAGCAAACTTGCTTGAAAGGAGGTCAACAA | |
| | | GAACAAGGAAATGTATAAGCTACAGATTTGATGAGTTTGA | |
| | | TGAAGCAATTGATGAAGCTATTGAAGATGACATCAAAGAA | |
| | | GCCGATGGAGGAGGAGTTGGCCGAGGAAAAGATATCTCCA | |
| | | CCATCACAGGTCATCGTGGGAAAGACATCTCTACTATTTT | |
| | | GGATGAAGAAAGAAAAGAAAATAAACGACCCCAGAGGGCA | |
| | | GCTGCTGCTCGAAGGAAGAAACGCCGGCGATTAAATGATC | |
| | | TGGACAGTGATAGCAACCTGGATGAAGAAGAGAGCGAGGA | |
| | | TGAATTCAAGATCAGTGATGGATCTCAAGATGAGTTTGTT | |
| | | GTGTCTGATGAAAACCCAGATGAAAGTGAAGAAGATCCGC | |
| | | CATCTAATGATGACAGTGACACTGACTTTTGTAGCCGTAG | |
| | | ACTGAGGCGACACCCCTCTCGGCCAATGAGGCAGAGCAGG | |
| | | CGTTTGCGAAGAAAGACCCCAAAGAAAAAATATTCCGATG | |
| | | ATGATGAAGAGGAGGAATCTGAGGAGAATAGTAGAGACTC | |
| | | TGAAAGTGACTTCAGTGATGATTTTAGTGATGATTTTGTA | |
| | | GAAACTCGGCGAAGGCGGTCAAGGAGAAATCAGAAAAGAC | |
| | | AAATTAACTACAAAGAAGACTCAGAAAGTGACGGTTCCCA | |
| | | GAAGAGTTTGCGACGTGGTAAAGAAATAAGGCGAGTACAC | |
| | | AAGCGAAGACTTTCCAGCTCAGAGAGTGAAGAGAGCTATT | |
| | | TGTCCAAGAACTCTGAAGATGATGAGCTAGCTAAAGAATC | |
| | | AAAGCGGTCAGTTCGAAAGCGGGGCCGAAGCACAGACGAG | |
| | | TATTCAGAAGCAGATGAGGAGGAGGAGGAAGAGGAAGGCA | |
| | | AACCATCCCGCAAACGGCTACACCGGATTGAGACGGATGA | |
| | | GGAGGAGAGTTGTGACAATGCTCATGGAGATGCAAATCAG | |
| | | CCTGCCCGTGACAGCCAGCCTAGGGTCCTGCCCTCAGAAC | |
| | | AAGAGAGCACCAAGAAGCCCTACCGGATAGAAAGTGATGA | |
| | | GGAAGAGGACTTTGAAAATGTAGGCAAAGTGGGGAGCCCA | |
| | | TTGGACTATAGCTTAGTGGACTTACCTTCAACCAATGGAC | |
| | | AGAGCCCTGGCAAAGCCATTGAGAACTTGATTGGCAAGCC | |
| | | TACTGAGAAGTCTCAGACCCCCAAGGACAACAGCACAGCC | |
| | | AGTGCAAGCCTAGCCTCCAATGGGACAAGTGGTGGGCAGG | |
| | | AGGCAGGAGCACCAGAAGAGGAGGAAGATGAGCTTTTGAG | |
| | | AGTGACTGACCTTGTTGATTATGTCTGTAACAGTGAACAG | |
| | | TTATAAGACTTTTTTTCCATTTTTGTGCTAATTTATTCCA | |
| | | CGGTAGCTCTCACACCAGCGGGCCAGTTATTAAAAGCTGT | |
| | | TTAATTTTTCCTAGAAAACTCCACTACAGAATGACTTTTA | |
| | | GAAGAAAAATTTCAACAAATCCTGAAGTCTTTCTGTGAAG | |
| | | TGACCAGTTCTGAACTTTGAAGATAAATAATTGCTGTAAA | |
| | | TTCCTTTTGATTTTCTTTTTCCAGGTTCATGGTCCTTGGT | |
| | | AATTTCATTCATGGAAAAAAATCTTATTATAATAACAACA | |
| | | AAGATTTGTATATTTTTGACTTTATATTTCCTGAGCTCTC | |
| | | CTGACTTTGTGAAAAAGGGTGGATGAAAATGCATTCCGAA | |
| | | TCTGTGAGGGCCCAAAACAGAATTTAGGGGTGGGTGAAAG | |
| | | CACTTGTGCTTTAGCTTTTTCATATTAAATATATATTATA | |
| | | TTTAAACATTCATGGCATAGATGATGATTTACAGACAATT | |
| | | TAAAAGTTCAAGTCTGTACTGTTACAGTTTGAGAATTGTA | |
| | | GATAACATCATACATAAGTCATTTAGTAACAGCCTTTGTG | |
| | | AAATGAACTTGTTTACTATTGGAGATAACCACACTTAATA | |
| | | AAGAAGAGACAGTGAAAGTACCATCATAATTAACCTAAAT | |
| | | TTTTGTTATAGCAGAGTTTCTTGTTTAAAAAAAAATAAAA | |
| | | TCATCTGAAAAGCAAAAA | |
| RTN2 | NM_005619.4 | CGCGCGCTGCAGTGCCTTCCCCACCTCGGCCCCGCCCGCC | 36 |
| | | CCCGCCGAGCCGAGCACCAGGGCGGCGGCGGCGGCGGCGG | |
| | | CGGCGGCGGCGGCTGGAGCAGCCCGGGAGGAGGAGGCGGC | |
| | | GAGAATGGCAGCGGCGTCGTGGGCGCGGCGGAGATGAGCG | |
| | | CCCGCGACCCCGGGCCCAGGGCGGCACAGCCGGAGTGGGC | |
| | | GGGGGTCCCGATGCAGGCCCGAGGGGGGCCATGGGGCAGG | |
| | | TCCTGCCGGTCTTCGCCCACTGCAAAGAAGCTCCGTCTAC | |
| | | AGCCTCCTCAACTCCTGATTCCACAGAAGGAGGGAACGAC | |
| | | GACTCTGATTTTCGAGAGCTGCACACAGCCCGGGAATTCT | |
| | | CAGAGGAGGACGAGGAGGAGACCACGTCGCAGGACTGGGG | |
| | | CACCCCCCGGGAGCTGACCTTCTCCTACATCGCCTTTGAT | |
| | | GGTGTAGTGGGCTCCGGGGGCCGCAGGGATTCAACTGCCC | |
| | | GCCGCCCCCGCCCCCAGGGCCGCTCAGTCTCGGAACCACG | |
| | | AGACCAGCACCCTCAGCCCAGCCTGGGCGACAGCTTGGAG | |
| | | AGCATCCCCAGCCTGAGCCAATCCCCGGAGCCTGGACGAC | |
| | | GGGGTGATCCTGACACCGCGCCTCCATCCGAGCGCCCTCT | |
| | | GGAAGACCTGAGGCTTCGGTTGGACCATCTGGGCTGGGTG | |
| | | GCCCGGGGAACGGGATCCGGGGAGGACTCTTCCACCAGCA | |
| | | GCTCCACCCCGCTGGAAGACGAAGAACCCCAAGAACCCAA | |
| | | CAGATTGGAGACAGGAGAAGCTGGGGAAGAACTGGACCTA | |
| | | CGACTCCGACTTGCTCAGCCCTCATCGCCCGAGGTCTTGA | |
| | | CTCCCCAGCTCAGTCCGGGCTCTGGGACACCCCAGGCCGG | |
| | | TACTCCGTCCCCATCCCGATCGCGAGATTCGAACTCTGGG | |
| | | CCCGAAGAGCCATTGCTGGAAGAGGAAGAAAAGCAGTGGG | |
| | | GGCCACTGGAGCGAGAGCCAGTAAGGGGACAGTGCCTCGA | |
| | | TAGCACGGACCAATTAGAATTCACGGTGGAGCCACGCCTT | |
| | | CTAGGAACAGCTATGGAATGGTTAAAGACATCATTGCTTT | |
| | | TGGCTGTTTACAAGACGGTTCCAATTTTGGAATTGTCCCC | |
| | | ACCTCTGTGGACAGCCATTGGCTGGGTCCAAAGGGGCCCC | |
| | | ACCCCCCCTACTCCTGTCCTCCGGGTTCTACTGAAGTGGG | |
| | | CAAAATCCCCGAGAAGCAGCGGTGTCCCCAGCCTCTCACT | |
| | | CGGAGCCGATATGGGGAGTAAAGTGGCGGACCTGCTGTAC | |
| | | TGGAAGGACACGAGGACGTCAGGAGTGGTCTTCACAGGCC | |
| | | TGATGGTCTCCCTCCTCTGCCTCCTGCACTTTAGCATCGT | |
| | | GTCCGTGGCCGCGCACTTGGCTCTGTTGCTGCTCTGCGGC | |
| | | ACCATCTCTCTCAGGGTTTACCGCAAAGTGCTGCAGGCCG | |
| | | TGCACCGGGGGGATGGAGCCAACCCTTTCCAGGCCTACCT | |
| | | GGATGTGGACCTCACCCTGACTCGGGAGCAGACGGAACGT | |
| | | TTGTCCCACCAGATCACCTCCCGCGTGGTCTCGGCGGCCA | |
| | | CGCAGCTGCGGCACTTCTTCCTGGTAGAAGACCTCGTGGA | |
| | | TTCCCTCAAGCTGGCCCTCCTCTTCTACATCTTGACCTTC | |
| | | GTGGGTGCCATCTTCAATGGTTTGACTCTTCTCATTCTGG | |
| | | GAGTGATTGGTCTATTCACCATCCCCCTGCTGTACCGGCA | |
| | | GCACCAGGCTCAGATCGACCAATATGTGGGGTTGGTGACC | |
| | | AATCAGTTGAGCCACATCAAAGCTAAGATCCGAGCTAAAA | |
| | | TCCCAGGGACCGGAGCCCTGGCCTCTGCAGCAGCCGCAGT | |
| | | CTCCGGATCCAAAGCCAAAGCCGAATGAGAACGGTGTCTC | |
| | | TGCCCGCAGGACGCCTGCCCCCAGCCCCCGCAGCCCTCTG | |
| | | GCCCCCTCCATCTCTTGTCCGTTCCCACCCACCCCCCTCC | |
| | | TCGGCCCGAGCCTTTTCCCGGTGGGTGTCAGGATCACTCC | |
| | | CACTAGGGACTCTGCGCTAATTACCTGAGCGACCAGGACT | |
| | | ACATTTCCCAAGAGGCTCTGCTCCAGGAGTCCAGGAAAGA | |
| | | CGAGGCACCTTGGCCGCGGGGCCTGCTGGGACTTGTAGTT | |
| | | GCCTAGACAGGGCACCACCCTGCACTTCCGGACCCGCCGC | |
| | | TGGAGGCGCCGTGAGGCGTTGGTGTCTCCTGGATGCTACT | |
| | | AGCCCCAACGCCGGGGCTTTGCATGGGGCCCAGGGGAGGC | |
| | | CTGAGCTTGGATTTACACTGTAATAAAGACTCCTGTGGAA | |
| | | AACCCGAG | |
| SMARCD3 | NM_001003801.1 | AGCAGGACTCAGAGGGGAGAGTTGGAGGAAAAAAAAAGGC | 37 |
| | | AGAAAAGGGAAAGAAAGAGGAAGAGAGAGAGAGAGTGAGA | |
| | | GGAGCCGCTGAGCCCACCCCGATGGCCGCGGACGAAGTTG | |
| | | CCGGAGGGGCGCGCAAAGCCACGAAAAGCAAACTTTTTGA | |
| | | GTTTCTGGTCCATGGGGTGCGCCCCGGGATGCCGTCTGGA | |
| | | GCCCGGATGCCCCACCAGGGGGCGCCCATGGGCCCCCCGG | |
| | | GCTCCCCGTACATGGGCAGCCCCGCCGTGCGACCCGGCCT | |
| | | GGCCCCCGCGGGCATGGAGCCCGCCCGCAAGCGAGCAGCG | |
| | | CCCCCGCCCGGGCAGAGCCAGGCACAGAGCCAGGGCCAGC | |
| | | CGGTGCCCACCGCCCCCGCGCGGAGCCGCAGTGCCAAGAG | |
| | | GAGGAAGATGGCTGACAAAATCCTCCCTCAAAGGATTCGG | |
| | | GAGCTGGTCCCCGAGTCCCAGGCTTACATGGACCTCTTGG | |
| | | CATTTGAGAGGAAACTGGATCAAACCATCATGCGGAAGCG | |
| | | GGTGGACATCCAGGAGGCTCTGAAGAGGCCCATGAAGCAA | |
| | | AAGCGGAAGCTGCGACTCTATATCTCCAACACTTTTAACC | |
| | | CTGCGAAGCCTGATGCTGAGGATTCCGACGGCAGCATTGC | |
| | | CTCCTGGGAGCTACGGGTGGAGGGGAAGCTCCTGGATGAT | |
| | | CCCAGCAAACAGAAGCGGAAGTTCTCTTCTTTCTTCAAGA | |
| | | GTTTGGTCATCGAGCTGGACAAAGATCTTTATGGCCCTGA | |
| | | CAACCACCTCGTTGAGTGGCATCGGACACCCACGACCCAG | |
| | | GAGACGGACGGCTTCCAGGTGAAACGGCCTGGGGACCTGA | |
| | | GTGTGCGCTGCACGCTGCTCCTCATGCTGGACTACCAGCC | |
| | | TCCCCAGTTCAAACTGGATCCCCGCCTAGCCCGGCTGCTG | |
| | | GGGCTGCACACACAGAGCCGCTCAGCCATTGTCCAGGCCC | |
| | | TGTGGCAGTATGTGAAGACCAACAGGCTGCAGGACTCCCA | |
| | | TGACAAGGAATACATCAATGGGGACAAGTATTTCCAGCAG | |
| | | ATTTTTGATTGTCCCCGGCTGAAGTTTTCTGAGATTCCCC | |
| | | AGCGCCTCACAGCCCTGCTATTGCCCCCTGACCCAATTGT | |
| | | CATCAACCATGTCATCAGCGTGGACCCTTCAGACCAGAAG | |
| | | AAGACGGCGTGCTATGACATTGACGTGGAGGTGGAGGAGC | |
| | | CATTAAAGGGGCAGATGAGCAGCTTCCTCCTATCCACGGC | |
| | | CAACCAGCAGGAGATCAGTGCTCTGGACAGTAAGATCCAT | |
| | | GAGACGATTGAGTCCATAAACCAGCTCAAGATCCAGAGGG | |
| | | ACTTCATGCTAAGCTTCTCCAGAGACCCCAAAGGCTATGT | |
| | | CCAAGACCTGCTCCGCTCCCAGAGCCGGGACCTCAAGGTG | |
| | | ATGACAGATGTAGCCGGCAACCCTGAAGAGGAGCGCCGGG | |
| | | CTGAGTTCTACCACCAGCCCTGGTCCCAGGAGGCCGTCAG | |
| | | TCGCTACTTCTACTGCAAGATCCAGCAGCGCAGGCAGGAG | |
| | | CTGGAGCAGTCGCTGGTTGTGCGCAACACCTAGGAGCCCA | |
| | | AAAATAAGCAGCACGACGGAACTTTCAGCCGTGTCCCGGG | |
| | | CCCCAGCATTTTGCCCCGGGCTCCAGCATCACTCCTCTGC | |
| | | CACCTTGGGGTGTGGGGCTGGATTAAAAGTCATTCATCTG | |
| | | ACAAAAAAAAAAAAAAAAAA | |
| SPATA7 | NM_001040428.3 | ACAATAGCGACTCACTGGACCCAGCCCTTAGCAACGGCCT | 38 |
| | | GGCGACGGTTTCCCTGCTGCTGCAGCCCCCGTCGGCTCCT | |
| | | CTTTTCCAGTCCTCCACTGCCGGGGCTGGGCCCGGCCGCG | |
| | | GGAAGGACCGAAGGGGATACAGCGTGTCCCTGCGGCGGCT | |
| | | GCAAGAGGACTAAGCATGGATGGCAGCCGGAGAGTCAGAG | |
| | | CAACCTCTGTCCTTCCCAGATATGGTCCACCGTGCCTATT | |
| | | TAAAGGACACTTGAGCACCAAAAGTAATGCTGCAGTAGAC | |
| | | TGCTCGGTTCCAGTAAGCGTGAGTACCAGCATAAAGTATG | |
| | | CAGACCAACAACGAAGAGAGAAACTCAAAAAGGAATTAGC | |
| | | ACAATGTGAAAAAGAGTTCAAATTAACTAAAACTGCAATG | |
| | | CGAGCCAATTATAAAAATAATTCCAAGTCACTTTTTAATA | |
| | | CCTTACAAAAGCCCTCAGGCGAACCGCAAATTGAGGATGA | |
| | | CATGTTAAAAGAAGAAATGAATGGATTTTCATCCTTTGCA | |
| | | AGGTCACTAGTACCCTCTTCAGAGAGACTACACCTAAGTC | |
| | | TACATAAATCCAGTAAAGTCATCACAAATGGTCCTGAGAA | |
| | | GAACTCCAGTTCCTCCCCGTCCAGTGTGGATTATGCAGCC | |
| | | TCCGGGCCCCGGAAACTGAGCTCTGGAGCCCTGTATGGCA | |
| | | GAAGGCCCAGAAGCACATTCCCAAATTCCCACCGGTTTCA | |
| | | GTTAGTCATTTCGAAAGCACCCAGTGGGGATCTTTTGGAT | |
| | | AAACATTCTGAACTCTTTTCTAACAAACAATTGCCATTCA | |
| | | CTCCTCGCACTTTAAAAACAGAAGCAAAATCTTTCCTGTC | |
| | | ACAGTATCGCTATTATACACCTGCCAAAAGAAAAAAGGAT | |
| | | TTTACAGATCAACGGATAGAAGCTGAAACCCAGACTGAAT | |
| | | TAAGCTTTAAATCTGAGTTGGGGACAGCTGAGACTAAAAA | |
| | | CATGACAGATTCAGAAATGAACATAAAGCAGGCATCTAAT | |
| | | TGTGTGACATATGATGCCAAAGAAAAAATAGCTCCTTTAC | |
| | | CTTTAGAAGGGCATGACTCAACATGGGATGAGATTAAGGA | |
| | | TGATGCTCTTCAGCATTCCTCACCAAGGGCAATGTGTCAG | |
| | | TATTCCCTGAAGCCCCCTTCAACTCGTAAAATCTACTCTG | |
| | | ATGAAGAAGAACTGTTGTATCTGAGTTTCATTGAAGATGT | |
| | | AACAGATGAAATTTTGAAACTTGGTTTATTTTCAAACAGG | |
| | | TTTTTAGAACGACTGTTCGAGCGACATATAAAACAAAATA | |
| | | AACATTTGGAGGAGGAAAAAATGCGCCACCTGCTGCATGT | |
| | | CCTGAAAGTAGACTTAGGCTGCACATCGGAGGAAAACTCG | |
| | | GTAAAGCAAAATGATGTTGATATGTTGAATGTATTTGATT | |
| | | TTGAAAAGGCTGGGAATTCAGAACCAAATGAATTAAAAAA | |
| | | TGAAAGTGAAGTAACAATTCAGCAGGAACGTCAACAATAC | |
| | | CAAAAGGCTTTGGATATGTTATTGTCGGCACCAAAGGATG | |
| | | AGAACGAGATATTCCCTTCACCAACTGAATTTTTCATGCC | |
| | | TATTTATAAATCAAAGCATTCAGAAGGGGTTATAATTCAA | |
| | | CAGGTGAATGATGAAACAAATCTTGAAACTTCAACTTTGG | |
| | | ATGAAAATCATCCAAGTATTTCAGACAGTTTAACAGATCG | |
| | | GGAAACTTCTGTGAATGTCATTGAAGGTGATAGTGACCCT | |
| | | GAAAAGGTTGAGATTTCAAATGGATTATGTGGTCTTAACA | |
| | | CATCACCCTCCCAATCTGTTCAGTTCTCCAGTGTCAAAGG | |
| | | CGACAATAATCATGACATGGAGTTATCAACTCTTAAAATC | |
| | | ATGGAAATGAGCATTGAGGACTGCCCTTTGGATGTTTAAT | |
| | | CTTCATTAATAAATACCTCAAATGGCCAGTAACTCAAAAA | |
| | | AAAAAAAAAAAAAAA | |
| SST1 | NM_001049.2 | TGGTCATCGCACGGCGGCAGCTCCTCACCTGGATTTAGAA | 39 |
| | | GAGCTGGCGTCCCCGCCCGCCCAAGCCTTTAAACTCTCGT | |
| | | CTGCCAGAACCCGCCAACTCTCCAGGCTTAGGGCCAGTTT | |
| | | CCGCGATTCTAAGAGTAATTGCGTGGGCACCTGTGCTGGG | |
| | | GCCAGGCGCAAAGAAGGGAGTTGGTCTGCGCGAAGATCGT | |
| | | CAACCTGCTAACAGACCGCACATGCACTTTGCACCGACCA | |
| | | TCTACGTCTCAGTCTGGAGGTTGCGCACTTTGGCTGCTGA | |
| | | CGCGCTGGTGGTGCCTATTAATCATTTACCAGTCCAGAGC | |
| | | CGCGCCAGTTAATGGCTGTGCCGTGCGGTGCTCCCACATC | |
| | | CTGGCCTCTCCTCTCCACGGTCGCCTGTGCCCGGGCACCC | |
| | | CGGAGCTGCAAACTGCAGAGCCCAGGCAACCGCTGGGCTG | |
| | | TGCGCCCCGCCGGCGCCGGTAGGAGCCGCGCTCCCCGCAG | |
| | | CGGTTGCGCTCTACCCGGAGGCGCTGGGCGGCTGTGGGCT | |
| | | GCAGGCAAGCGGTCGGGTGGGGAGGGAGGGCGCAGGCGGC | |
| | | GGGTGCGCGAGGAGAAAGCCCCAGCCCTGGCAGCCCCACT | |
| | | GGCCCCCCTCAGCTGGGATGTTCCCCAATGGCACCGCCTC | |
| | | CTCTCCTTCCTCCTCTCCTAGCCCCAGCCCGGGCAGCTGC | |
| | | GGCGAAGGCGGCGGCAGCAGGGGCCCCGGGGCCGGCGCTG | |
| | | CGGACGGCATGGAGGAGCCAGGGCGAAATGCGTCCCAGAA | |
| | | CGGGACCTTGAGCGAGGGCCAGGGCAGCGCCATCCTGATC | |
| | | TCTTTCATCTACTCCGTGGTGTGCCTGGTGGGGCTGTGTG | |
| | | GGAACTCTATGGTCATCTACGTGATCCTGCGCTATGCCAA | |
| | | GATGAAGACGGCCACCAACATCTACATCCTAAATCTGGCC | |
| | | ATTGCTGATGAGCTGCTCATGCTCAGCGTGCCCTTCCTAG | |
| | | TCACCTCCACGTTGTTGCGCCACTGGCCCTTCGGTGCGCT | |
| | | GCTCTGCCGCCTCGTGCTCAGCGTGGACGCGGTCAACATG | |
| | | TTCACCAGCATCTACTGTCTGACTGTGCTCAGCGTGGACC | |
| | | GCTACGTGGCCGTGGTGCATCCCATCAAGGCGGCCCGCTA | |
| | | CCGCCGGCCCACCGTGGCCAAGGTAGTAAACCTGGGCGTG | |
| | | TGGGTGCTATCGCTGCTCGTCATCCTGCCCATCGTGGTCT | |
| | | TCTCTCGCACCGCGGCCAACAGCGACGGCACGGTGGCTTG | |
| | | CAACATGCTCATGCCAGAGCCCGCTCAACGCTGGCTGGTG | |
| | | GGCTTCGTGTTGTACACATTTCTCATGGGCTTCCTGCTGC | |
| | | CCGTGGGGGCTATCTGCCTGTGCTACGTGCTCATCATTGC | |
| | | TAAGATGCGCATGGTGGCCCTCAAGGCCGGCTGGCAGCAG | |
| | | CGCAAGCGCTCGGAGCGCAAGATCACCTTAATGGTGATGA | |
| | | TGGTGGTGATGGTGTTTGTCATCTGCTGGATGCCTTTCTA | |
| | | CGTGGTGCAGCTGGTCAACGTGTTTGCTGAGCAGGACGAC | |
| | | GCCACGGTGAGTCAGCTGTCGGTCATCCTCGGCTATGCCA | |
| | | ACAGCTGCGCCAACCCCATCCTCTATGGCTTTCTCTCAGA | |
| | | CAACTTCAAGCGCTCTTTCCAACGCATCCTATGCCTCAGC | |
| | | TGGATGGACAACGCCGCGGAGGAGCCGGTTGACTATTACG | |
| | | CCACCGCGCTCAAGAGCCGTGCCTACAGTGTGGAAGACTT | |
| | | CCAACCTGAGAACCTGGAGTCCGGCGGCGTCTTCCGTAAT | |
| | | GGCACCTGCACGTCCCGGATCACGACGCTCTGAGCCCGGG | |
| | | CCACGCAGGGGCTCTGAGCCCGGGCCACGCAGGGGCCCTG | |
| | | AGCCAAAAGAGGGGGAGAATGAGAAGGGAAGGCCGGGTGC | |
| | | GAAAGGGACGGTATCCAGGGCGCCAGGGTGCTGTCGGGAT | |
| | | AACGTGGGGCTAGGACACTGACAGCCTTTGATGGAGGAAC | |
| | | CCAAGAAAGGCGCGCGACAATGGTAGAAGTGAGAGCTTTG | |
| | | CTTATAAACTGGGAAGGCTTTCAGGCTACCTTTTTCTGGG | |
| | | TCTCCCACTTTCTGTTCCTTCCTCCACTGCGCTTACTCCT | |
| | | CTGACCCTCCTTCTATTTTCCCTACCCTGCAACTTCTATC | |
| | | CTTTCTTCCGCACCGTCCCGCCAGTGCAGATCACGAACTC | |
| | | ATTAACAACTCATTCTGATCCTCAGCCCCTCCAGTCGTTA | |
| | | TTTCTGTTTGTTTAAGCTGAGCCACGGATACCGCCACGGG | |
| | | TTTCCCTCGGCGTTAGTCCCTAGCCGCGCGGGGCCGCTGT | |
| | | CCAGGTTCTGTCTGGTGCCCCTACTGGAGTCCCGGGAATG | |
| | | ACCGCTCTCCCTTTGCGCAGCCCTACCTTAAGGAAAGTTG | |
| | | GACTTGAGAAAGATCTAAGCAGCTGGTCTTTTCTCCTACT | |
| | | CTTGGGTGAAGGTGCATCTTTCCCTGCCCTCCCCTGTCCC | |
| | | CCTCTCGCCGCCCGCCCGCCACCACCACTCTCACTCCACC | |
| | | CAGAGTAGAGCCAGGTGCTTAGTAAAATAGGTCCCGCGCT | |
| | | TCGAACTCCAGGCTTTCTGGAGTTCCCACCCAAGCCCTCC | |
| | | TTTGGAGCAAAGAAGGAGCTGAGAACAAGCCGAATGAGGA | |
| | | GTTTTTATAAGATTGCGGGGTCGGAGTGTGGGCGCGTAAT | |
| | | AGGAATCACCCTCCTACTGCGCGTTTTCAAAGACCAAGCG | |
| | | CTGGGCGCTCCCGGGCCGCGCGTCTGCGTTAGGCAGGGCA | |
| | | GGGTAGTGCAGGGCACACCTTCCCCGGGGTTCGGGGTTCG | |
| | | GGGTTCGGTTGCAGGGCTGCAGCCCGCCTTGGCTTTCTCC | |
| | | CTCACCCAAGTTTCCGGAGGAGCCGACCTAAAAGTAACAA | |
| | | TAGATAAGGTTTCCTGCTCCAGTGTATCTCAAAAGACCGG | |
| | | GCGCCAGGGGCGGGGGACCTAGGGCGACGTCTTCAGAGTC | |
| | | CGCCAGTGTTGGCGGTGTCGCCGCAACCTGCAGGCTCCCG | |
| | | AGTGGGGCCTGCCTGGTCTCTAGAGGGTTGCTGCCTTTCA | |
| | | AGCGGTGCCTAAGAAGTTATTTTCTTGTTTAACATATATA | |
| | | TTTATTAATTTATTTGTCGTGTTGGAAAATGTGTCTCTGC | |
| | | TTTCCTTTTCTCTGCTTGCCTAGCCCCAGGTCTTTTCTTT | |
| | | GGGACCCTGGGGGCGGGCATGGAAGTGGAAGTAGGGGCAA | |
| | | GCTCTTGCCCCACTCCCTGGCCATCTCAACGCCTCTCCTC | |
| | | AATGCTGGGCCCTCTTATCTCATCCTTTCCTCTAGCTTTT | |
| | | CTATTTTTGATTGTGTTGAGTGAAGTTTGGAGATTTTTCA | |
| | | TACTTTTCTTACTATAGTCTCTTGTTTGTCTTATTAGGAT | |
| | | AATACATAAATGATAATGTGGGTTATCCTCCTCTCCATGC | |
| | | ACAGTGGAAAGTCCTGAACTCCTGGCTTTCCAGGAGACAT | |
| | | ATATAGGGGAACATCACCCTATATATAATTTGAGTGTATA | |
| | | TATATTTATATATATGATGTGGACATATGTATACTTATCT | |
| | | TGCTCCATTGTCATGAGTCCATGAGTCTAAGTATAGCCAC | |
| | | TGATGGTGACAGGTGTGAGTCTGGCTGGAACACTTTCAGT | |
| | | TTCAGGAGTGCAAGCAGCACTCAAACCTGGAGCTGAGGAA | |
| | | TCTAATTCAGACAGAGACTTTAATCACTGCTGAAGATGCC | |
| | | CCTGCTCCCTCTGGGTTCCAGCAGAGGTGATTCTTACATA | |
| | | TGATCCAGTTAACATCATCACTTTTTTTGAGGACATTGAA | |
| | | AGTGAAATAATTTGTGTCTGTGTTTAATATTACCAACTAC | |
| | | ATTGGAAGCCTGAGCAGGGCGAGGACCAATAATTTTAATT | |
| | | ATTTATATTTCCTGTATTGCTTTAGTATGCTGGCTTGTAC | |
| | | ATAGTAGGCACTAAATACATGTTTGTTGGTTGATTGTTTA | |
| | | AGCCAGAGTGTATTACAACAATCTGGAGATACTAAATCTG | |
| | | GGGTTCTCAGGTTCACTCATTGACATGATATACAATGGTT | |
| | | AAAATCACTATTGAAAAATACGTTTTGTGTATATTTGCTT | |
| | | CAACAACTTTGTGCTTTCCTGAAAGCAGTAACCAAGAGTT | |
| | | AAGATATCCCTAATGTTTTGCTTAAACTAATGAACAAATA | |
| | | TGCTTTGGGTCATAAATCAGAAAGTTTAGATCTGTCCCTT | |
| | | AATAAAAATATATATTACTACTCCTTTGGAAAATAGATTT | |
| | | TTAATGGTTAAGAACTGTGAAATTTACAAATCAAAATCTT | |
| | | AATCATTATCCTTCTAAGAGGATACAAATTTAGTGCTCTT | |
| | | AACTTGTTACCATTGTAATATTAACTAAATAAACAGATGT | |
| | | ATTATGCTGTTAAAAAAAAAAAAAAAAAAAAAAAAAAAAA | |
| | | AAAAAAAAAAAAAAAAAAAAAAA | |
| SST3 | NM_001051.4 | CTGCATCTCTCCCTCTCACCCGTGTCTCCTCTCCTCTCTT | 40 |
| | | TCCTTCTCGTCTTCTCCCTGTCACGCATCTCTCATCACTC | |
| | | CCCCTCATTCTGCCTTTCCTCCTACTCACGGTCTCCTCTC | |
| | | CCTCTCCCTCTCTCTCTCTCCCCCTCCCTCTTTCTCTCTC | |
| | | TCTCTCTTTCTCCACCTCCTCCCGACCCCCTTTCCCCTCT | |
| | | ATTTCTATTGGCTTCTGTGTCCCTTGCTCCCCTCTTCTCT | |
| | | TCCTCACCCTGGGAAGCTTCTCCCCCCTATCCTTGCCCCT | |
| | | GCCCCCCCAGGATGTGTCCTGGAGATGGGGGGTGACGTAC | |
| | | CAGGCTCTGGTTGGGAAGTCAGGGCCGGAGACCAGATGGG | |
| | | AGAGGCTCTGTGGACAGCCGTGGCCGAGGGCCTGGGAGGG | |
| | | AACCTGAGCCCGCAAGCGGTCTAGAAGTGGGTGCCTTGTG | |
| | | GGGACCCTAGTTAGGAGTGCCCTGGGGGCACCTGGGGACT | |
| | | GGGCAGGGAGAGGGGACAGCAGAATGATAACCAGCCTGGC | |
| | | GGCAAGGAGGGAAGCCCTCACCCCATGGGCAGGCAAATAG | |
| | | CTGACTGCTGACCACCCTCCCCTCAGCCATGGACATGCTT | |
| | | CATCCATCATCGGTGTCCACGACCTCAGAACCTGAGAATG | |
| | | CCTCCTCGGCCTGGCCCCCAGATGCCACCCTGGGCAACGT | |
| | | GTCGGCGGGCCCAAGCCCGGCAGGGCTGGCCGTCAGTGGC | |
| | | GTTCTGATCCCCCTGGTCTACCTGGTGGTGTGCGTGGTGG | |
| | | GCCTGCTGGGTAACTCGCTGGTCATCTATGTGGTCCTGCG | |
| | | GCACACGGCCAGCCCTTCAGTCACCAACGTCTACATCCTC | |
| | | AACCTGGCGCTGGCCGACGAGCTCTTCATGCTGGGGCTGC | |
| | | CCTTCCTGGCCGCCCAGAACGCCCTGTCCTACTGGCCCTT | |
| | | CGGCTCCCTCATGTGCCGCCTGGTCATGGCGGTGGATGGC | |
| | | ATCAACCAGTTCACCAGCATATTCTGCCTGACTGTCATGA | |
| | | GCGTGGACCGCTACCTGGCCGTGGTACATCCCACCCGCTC | |
| | | GGCCCGCTGGCGCACAGCTCCGGTGGCCCGCACGGTCAGC | |
| | | GCGGCTGTGTGGGTGGCCTCAGCCGTGGTGGTGCTGCCCG | |
| | | TGGTGGTCTTCTCGGGAGTGCCCCGCGGCATGAGCACCTG | |
| | | CCACATGCAGTGGCCCGAGCCGGCGGCGGCCTGGCGAGCC | |
| | | GGCTTCATCATCTACACGGCCGCACTGGGCTTCTTCGGGC | |
| | | CGCTGCTGGTCATCTGCCTCTGCTACCTGCTCATCGTGGT | |
| | | GAAGGTGCGCTCAGCTGGGCGCCGGGTGTGGGCACCCTCG | |
| | | TGCCAGCGGCGGCGGCGCTCCGAACGCAGGGTCACGCGCA | |
| | | TGGTGGTGGCCGTGGTGGCGCTCTTCGTGCTCTGCTGGAT | |
| | | GCCCTTCTACGTGCTCAACATCGTCAACGTGGTGTGCCCA | |
| | | CTGCCCGAGGAGCCTGCCTTCTTTGGGCTCTACTTCCTGG | |
| | | TGGTGGCGCTGCCCTATGCCAACAGCTGTGCCAACCCCAT | |
| | | CCTTTATGGCTTCCTCTCCTACCGCTTCAAGCAGGGCTTC | |
| | | CGCAGGGTCCTGCTGCGGCCCTCCCGCCGTGTGCGCAGCC | |
| | | AGGAGCCCACTGTGGGGCCCCCGGAGAAGACTGAGGAGGA | |
| | | GGATGAGGAGGAGGAGGATGGGGAGGAGAGCAGGGAGGGG | |
| | | GGCAAGGGGAAGGAGATGAACGGCCGGGTCAGCCAGATCA | |
| | | CGCAGCCTGGCACCAGCGGGCAGGAGCGGCCGCCCAGCAG | |
| | | AGTGGCCAGCAAGGAGCAGCAGCTCCTACCCCAAGAGGCT | |
| | | TCCACTGGGGAGAAGTCCAGCACGATGCGCATCAGCTACC | |
| | | TGTAGGGGCCTGGGGAAAGCCAGGATGGCCCGAGGAAGAG | |
| | | GCAGAAGCCGTGGGTGTGCCTAGGGCCTACTTCCCAAGGT | |
| | | GCCACAGGCCCATGATGGGATGTTGAGGGGCCTGGACTTT | |
| | | GATGCTATTGCTGCCAGGTCTTGCTGTGTGACCTTGGGTA | |
| | | GGTTGCTTCTACTCTCTGGGCCTTGTTTTCTCCTCTGTGA | |
| | | CTCAGGGATAGGAGTCATCAGCCTGGATGAGCTATGTCAG | |
| | | ATGAGAGGTTTGGAGGGCACTGTTGCTGGGCTGACCTGGC | |
| | | TGAGCAGGCAAAAGGTGGGTGCAGACTGGCCTCCCCCCAG | |
| | | GGATGGAGTGTCTTGGGGCATCAACTAGAATCTTGGCCCT | |
| | | CAGAGGGATAAACCAAGGCCAGGATTTCTTGGGCTCAGAG | |
| | | TCAGGAACACAGGAGCTGCTGGGGGCTGGGCTGGAAACCT | |
| | | AAACAGAAGAAAGCCTAACCCGGTGGGAGGAGTGGGGCAG | |
| | | AAATGGTCAGGCCCCAGATCAGCTCCCTCCCCTCGACTGT | |
| | | GAGGCCTTGGACCAGCTCTGCTCCTCTCTAGGCCTCAGGC | |
| | | TTCACCTGGGTAAAACCCAACAACCTCTACACCCTTTTGG | |
| | | CCCAGGCAGTCAATGCTGGAGGTCCTGTGCTCCTGGACGG | |
| | | GAAGAGCAGGTGAATTTCCTGCTCATGGAAGCGAATGAAG | |
| | | TCCAGCTTCAGGGTCTCTCACTGCCTGGGCTTTTGCAAGG | |
| | | CCCTGCATCTACTTTTGTACTTGTCATTTTGTATTCGTTT | |
| | | TCTTAAAGAGGGACCTCGAACTGCATAAGCTTAGGCCACC | |
| | | CAAAGCCTGGCTCTGCCCCTGCTGAGGTCAGCCACCCAAT | |
| | | CCCCAAGGAAGCTCATGTTGGGTCTTATGGCTGGAGTAGG | |
| | | GGCCCCCGGGGGTTCCCAGGTCTTTTGAGGGCTTCCAGGC | |
| | | ACCTCCTTGTAGGAAGGGCCATCCCTGTTCCTCTCCTTGT | |
| | | GACCCATATTCTCCCTTCCTGGAGACCGAGACAGGGACCC | |
| | | AGCCCATGAGGACTGGCATGGAAAGGCAGAGTGTCTGAAG | |
| | | AGCGCTGTGAGGAGAAGGAAGAGGAAGGGAGAAGAGGAAG | |
| | | AGGAAGGAGAAGGAAGAGGAAGACAAGGGGGAAAGGGGAG | |
| | | GATGAGGAGGGGGAAGGAGAAGTACAGATCTGTTTCCTGG | |
| | | AGCCGTCTTTGGCCCCCCTGGGCTGAGCTCAGTGGTAGCA | |
| | | TCTGTGAACCTGAGTTGCCGACAACAGCCCCACCCAACCA | |
| | | GTACTGAGGGAAGGACACGATCAGGGTGGAACAGCCAGGG | |
| | | TGCAATGGCAAATGCACAGAGTACAGACAGGCACAGGGCC | |
| | | TGCGTCCCTGAGGGGCCTCAGAGTGCTGCCAAGAGGGCTC | |
| | | AGGCCTTAATAAAGCCCTAGGGTGGAGCTGGCTACCAGGG | |
| | | ACATTGGGAGGACTGGGGAGCTCCCTCCCCATGCTCTATC | |
| | | ATCCTGGAGACTACAGGTCGGGAGGCCCAGGGAAGACAAG | |
| | | AAGAGGCTGAAGTGGGACTGTGGAGGGGGACCATGGGGAG | |
| | | CAGCCACCATCCAAGGCTGGGCCTAGACTCCCTCCCAGAG | |
| | | ATGGTCCCTCAGAGCTGTGGTGAGGCTGGCCCTGGGAGGG | |
| | | TGAGACCCCCGGTGAAATCCTTCCGCTTCCCCACCCCTTG | |
| | | CAGAGGGCAGGGGTCCTCAGGGAAAGCACAGGAACCAGAC | |
| | | TTTTGGAGACTTGGATCTTCAGCACACCTCAGGGTCCTGG | |
| | | GCTGGCATTGGCCTTCCGGGCCTCAATTTCCCCATCAACA | |
| | | AATGGAGATGAATCCCAGCTTGGCTGCCTCCTGGGATCTA | |
| | | ACGAGAAAATGAGTCATGTGAGGTAACTTCCAGGCTCACT | |
| | | GCAATGGGTACGGTGGGGTGTATCAGATTATAAAGTGGGG | |
| | | GTGCCCTCCTCACCCCCAGGCTTGGCCTATACCCCCCTCT | |
| | | CCATCAAGTGGCCTCTCTGTGTCTGTCCTTTGGGGTGAGG | |
| | | ACACTGTAGGCCATGAGAAATGGGCAGTTGGGGGGTCAGA | |
| | | GGCCAAGGGTTAGGGAGGCAGGGCTTGGGGAGAGTGTGGG | |
| | | ACCATCAGAAGAGAAGGAAGTTTACAAAACCACATTTTGT | |
| | | GTGGAGATGGAGGCTGGAGGCCCGGCCCTGGGACTTGGTC | |
| | | TGGGGTTTCTTGAGGAAGATCTGAGGGTCCAAGGGAGGAA | |
| | | GGATGCCCTGGCCTTCTGGCCTTCTCTGGCTGATCCTGCC | |
| | | TTCTTGCTGCCTAGGACAGGAGAGTAATGTCCTAGAATGG | |
| | | TCCCTGGGAGGCCAGTTAGGAAACCCTTTGCTGCTTCTGT | |
| | | CTCTAGCTCTTGTCAATAAAGACGGTGACACCTGAAAAAA | |
| | | AAAAAAAAAAA | |
| SST4 | NM_001052.2 | CCGAGCTCTCTGGCGCAGCGCTAGCTCCGCCGCGCTCAGC | 41 |
| | | TGCCCTGCGCCGGCACCCCTGGTCATGAGCGCCCCCTCGA | |
| | | CGCTGCCCCCCGGGGGCGAGGAAGGGCTGGGGACGGCCTG | |
| | | GCCCTCTGCAGCCAATGCCAGTAGCGCTCCGGCGGAGGCG | |
| | | GAGGAGGCGGTGGCGGGGCCCGGGGACGCGCGGGCGGCGG | |
| | | GCATGGTCGCTATCCAGTGCATCTACGCGCTGGTGTGCCT | |
| | | GGTGGGGCTGGTGGGCAACGCCCTGGTCATCTTCGTGATC | |
| | | CTTCGCTACGCCAAGATGAAGACGGCTACCAACATCTACC | |
| | | TGCTCAACCTGGCCGTAGCCGACGAGCTCTTCATGCTGAG | |
| | | CGTGCCCTTCGTGGCCTCGTCGGCCGCCCTGCGCCACTGG | |
| | | CCCTTCGGCTCCGTGCTGTGCCGCGCGGTGCTCAGCGTCG | |
| | | ACGGCCTCAACATGTTCACCAGCGTCTTCTGTCTCACCGT | |
| | | GCTCAGCGTGGACCGCTACGTGGCCGTGGTGCACCCTCTG | |
| | | CGCGCGGCGACCTACCGGCGGCCCAGCGTGGCCAAGCTCA | |
| | | TCAACCTGGGCGTGTGGCTGGCATCCCTGTTGGTCACTCT | |
| | | CCCCATCGCCATCTTCGCAGACACCAGACCGGCTCGCGGC | |
| | | GGCCAGGCCGTGGCCTGCAACCTGCAGTGGCCACACCCGG | |
| | | CCTGGTCGGCAGTCTTCGTGGTCTACACTTTCCTGCTGGG | |
| | | CTTCCTGCTGCCCGTGCTGGCCATTGGCCTGTGCTACCTG | |
| | | CTCATCGTGGGCAAGATGCGCGCCGTGGCCCTGCGCGCTG | |
| | | GCTGGCAGCAGCGCAGGCGCTCGGAGAAGAAAATCACCAG | |
| | | GCTGGTGCTGATGGTCGTGGTCGTCTTTGTGCTCTGCTGG | |
| | | ATGCCTTTCTACGTGGTGCAGCTGCTGAACCTCTTCGTGA | |
| | | CCAGCCTTGATGCCACCGTCAACCACGTGTCCCTTATCCT | |
| | | TAGCTATGCCAACAGCTGCGCCAACCCCATTCTCTATGGC | |
| | | TTCCTCTCCGACAACTTCCGCCGATTCTTCCAGCGGGTTC | |
| | | TCTGCCTGCGCTGCTGCCTCCTGGAAGGTGCTGGAGGTGC | |
| | | TGAGGAGGAGCCCCTGGACTACTATGCCACTGCTCTCAAG | |
| | | AGCAAAGGTGGGGCAGGGTGCATGTGCCCCCCACTCCCCT | |
| | | GCCAGCAGGAAGCCCTGCAACCAGAACCCGGCCGCAAGCG | |
| | | CATCCCCCTCACCAGGACCACCACCTTCTGAGGAGCCCTT | |
| | | CCCCTACCCACCCTGCGT | |
| SST5 | NM_001053.3 | ATGCCTGCATGTGCTGGTTCAGGGACTCACCACCCTGGCG | 42 |
| | | TCCTCCCTTCTTCTCTTGCAGAGCCTGACGCACCCCAGGG | |
| | | CTGCCGCCATGGAGCCCCTGTTCCCAGCCTCCACGCCCAG | |
| | | CTGGAACGCCTCCTCCCCGGGGGCTGCCTCTGGAGGCGGT | |
| | | GACAACAGGACGCTGGTGGGGCCGGCGCCCTCGGCAGGGG | |
| | | CCCGGGCGGTGCTGGTGCCCGTGCTGTACCTGCTGGTGTG | |
| | | TGCGGCCGGGCTGGGCGGGAACACGCTGGTCATCTACGTG | |
| | | GTGCTGCGCTTCGCCAAGATGAAGACCGTCACCAACATCT | |
| | | ACATTCTCAACCTGGCAGTGGCCGACGTCCTGTACATGCT | |
| | | GGGGCTGCCTTTCCTGGCCACGCAGAACGCCGCGTCCTTC | |
| | | TGGCCCTTCGGCCCCGTCCTGTGCCGCCTGGTCATGACGC | |
| | | TGGACGGCGTCAACCAGTTCACCAGTGTCTTCTGCCTGAC | |
| | | AGTCATGAGCGTGGACCGCTACCTGGCAGTGGTGCACCCG | |
| | | CTGAGCTCGGCCCGCTGGCGCCGCCCGCGTGTGGCCAAGC | |
| | | TGGCGAGCGCCGCGGCCTGGGTCCTGTCTCTGTGCATGTC | |
| | | GCTGCCGCTCCTGGTGTTCGCGGACGTGCAGGAGGGCGGT | |
| | | ACCTGCAACGCCAGCTGGCCGGAGCCCGTGGGGCTGTGGG | |
| | | GCGCCGTCTTCATCATCTACACGGCCGTGCTGGGCTTCTT | |
| | | CGCGCCGCTGCTGGTCATCTGCCTGTGCTACCTGCTCATC | |
| | | GTGGTGAAGGTGAGGGCGGCGGGCGTGCGCGTGGGCTGCG | |
| | | TGCGGCGGCGCTCGGAGCGGAAGGTGACGCGCATGGTGTT | |
| | | GGTGGTGGTGCTGGTGTTTGCGGGATGTTGGCTGCCCTTC | |
| | | TTCACCGTCAACATCGTCAACCTGGCCGTGGCGCTGCCCC | |
| | | AGGAGCCCGCCTCCGCCGGCCTCTACTTCTTCGTGGTCAT | |
| | | CCTCTCCTACGCCAACAGCTGTGCCAACCCCGTCCTCTAC | |
| | | GGCTTCCTCTCTGACAACTTCCGCCAGAGCTTCCAGAAGG | |
| | | TTCTGTGCCTCCGCAAGGGCTCTGGTGCCAAGGACGCTGA | |
| | | CGCCACGGAGCCGCGTCCAGACAGGATCCGGCAGCAGCAG | |
| | | GAGGCCACGCCACCCGCGCACCGCGCCGCAGCCAACGGGC | |
| | | TTATGCAGACCAGCAAGCTGTGAGAGTGCAGGCGGGGGGT | |
| | | GGGCGGCCCCGTGTCACCCCCAGGAGCGGAGGTTGCACTG | |
| | | CGGTGACCCCCACCCATGACCTGCCAGTCAGGATGCTCCC | |
| | | CGGCGGTGGTGTGAGGACAGAGCTGGCTGAAGCCAGGCTG | |
| | | GGGTAGACACAGGGCAGTAGGTTCCCCACCGTGACCGACC | |
| | | ATCCCCTCTAACCGTCTGCCACACAGCGGGGGCTCCCGGG | |
| | | AGGTAGGGGAGGTGGCCAGACCGGTGGGGGGCTCCGCCAT | |
| | | GCCGTGCAAGTGCTCAGGGCCGCCTCACCCTCCATCTGGC | |
| | | CCCAGCCCATGCCGGCCTTCCCTCTGGGGAGCGACTTTTC | |
| | | CAGAAGGCCGGCCAGGCGAGAGGGTCTTCCTGACGGCGGA | |
| | | GCTGACCTGCCCGGCCCACCAGCTGCATGTCAGCTCCGAG | |
| | | CCACCGGGTCCCCGTCCAAGGCTGCTCTGCTAAGTTAAAG | |
| | | ACACCCGAAAGCGCTTGACTCAGGTCCCCGGAGTCCCTGG | |
| | | CCAGGGCCCCAGCCCCTCGCTTGCCCTGCACTGTGTGGAC | |
| | | TCTGGGGATGCAGGTGTAAGGGGAGTGTGGCTGGGCAGCC | |
| | | CCTGGTCAGCCAGGGTCACGCCTGTCCTGGGGGCCCCACC | |
| | | CTGCTGCCCGACACCCCCCATGGGAGGCTGCGGGCGGCAG | |
| | | TTGCTGTCTCAGAGAGGGGAGTGTGGGGGCTTGGGCGCTG | |
| | | GCCTAGCCAGGGGCGAGGTGGGGAGGCGGCTGGTGCAGAG | |
| | | GAGAGCTGGGGGCTGAGGTTGGGGTGAAGGCTGCAGCCCT | |
| | | CCAGGCTGCTGGGGGTGCAGATGGCTGTGCCGTGCTGAGA | |
| | | TTGGCTCTGTCTGGAGGGGTCCAGTGTGGGGTGCCTGAGG | |
| | | GCACTAGGGAGAGGTGCTCCTGCTGCAGGAGGACCTGAGG | |
| | | GTCAGGGCTTGGAGAGGACAGGGAACCTGCGGCCGTCTCT | |
| | | TCTGCTTTGGGGCAGGGGCTCTGGCCCGGGAGAGGGAACG | |
| | | GGGACAGGAGCAGAGGACGGTCATCCAGGCGCAGCGGGGA | |
| | | GCTGCTCCCCAGGCCACAGCAGACAGCACTGCTGAGAGGC | |
| | | AGCGGCCGCGCGGGTGACGCAAATGGCAGGCCCTGGGAAT | |
| | | CCCGCCGCCTCCCACCTAGAATTGTCCTACCTCCCCCACC | |
| | | CCAAACACCAGCTTTTCCTGGCGCCCCAGGCCCAGAACGT | |
| | | GGGCCCAGAGAGCCTTGCTGGGGTCTCTGGGGCACCTTGG | |
| | | CCTTGCTCTGAGGCTGGAAGGAGAAGGACCAGGGTGCGGC | |
| | | ATCACTCGGCCTCAGGGACCCCTCTGCCCTGCCCAGCACT | |
| | | GGCCCCGACCCGTGCTCCCGCCGTCTGCCCAGAGCAGGAC | |
| | | CTCAACCTCCTGGAGGGCACAGGGAGCGGCTGAGTGGGCA | |
| | | CAAATCCTGGCAGGAGAAAGGCCCAGGCTGAGGCCAGGCC | |
| | | TGGGAAACATCCAAGCAGTGAGGACACGCGTGTTTGACAA | |
| | | CTGCTCCCCTGAATAAATGCGAGGATAAATGTTT | |
| TECPR2 | NM_001172631.1 | CCCCCGGCGGAGCCAGCTGCTGCTCTTCGGTGCTGGCCCC | 43 |
| | | GGTGCCGGCCCCGTTGCCCAGGGAACAGGCTCCCGGCAGC | |
| | | CCCCGCGGCCCGGAGTCCATCCCGCCTCCTCCGGCCCGGC | |
| | | GGGGCCGACGAGTCCGGAGGGGCTGCCGCGGGAGCCCCCA | |
| | | GGTTTCCCTAGATGACAAATAAACATTCCTTTTCCTGCGT | |
| | | GAAGATAGTCTGTGGAAACCTTGGCCATGGCATCGATATC | |
| | | AGAGCCTGTTACATTCAGAGAGTTCTGCCCGTTGTACTAT | |
| | | CTCCTCAATGCCATTCCGACAAAGATCCAGAAGGGTTTCC | |
| | | GCTCTATCGTGGTCTATCTCACGGCCCTCGACACCAACGG | |
| | | GGACTACATCGCGGTGGGCAGCAGCATCGGCATGCTCTAT | |
| | | CTGTACTGCCGGCACCTCAACCAGATGAGGAAGTACAACT | |
| | | TTGAGGGGAAGACGGAATCTATCACTGTGGTGAAGCTGCT | |
| | | GAGCTGCTTTGATGACCTGGTGGCAGCAGGCACAGCCTCT | |
| | | GGCAGGGTTGCAGTTTTTCAACTTGTATCTTCATTGCCAG | |
| | | GGAGAAATAAACAGCTTCGGAGATTTGATGTCACTGGTAT | |
| | | TCACAAAAATAGCATTACAGCTCTGGCTTGGAGCCCCAAT | |
| | | GGAATGAAATTGTTCTCTGGAGATGACAAAGGCAAAATTG | |
| | | TTTATTCTTCTCTGGATCTAGACCAGGGGCTCTGTAACTC | |
| | | CCAGCTGGTGTTGGAGGAGCCATCTTCCATTGTGCAGCTG | |
| | | GATTATAGCCAGAAAGTGCTGCTGGTCTCTACTCTGCAAA | |
| | | GAAGTCTGCTCTTTTACACTGAAGAAAAGTCTGTAAGGCA | |
| | | AATTGGAACACAACCAAGGAAAAGTACTGGGAAATTTGGT | |
| | | GCTTGTTTTATACCAGGACTCTGTAAGCAAAGTGATCTAA | |
| | | CCTTGTATGCGTCACGGCCCGGGCTCCGGCTATGGAAGGC | |
| | | TGATGTCCACGGGACTGTTCAAGCCACGTTTATCTTAAAA | |
| | | GATGCTTTTGCCGGGGGAGTCAAGCCTTTTGAACTGCACC | |
| | | CGCGTCTGGAATCCCCCAACAGTGGAAGTTGCAGCTTACC | |
| | | TGAGAGGCACCTGGGGCTTGTTTCATGTTTCTTTCAAGAA | |
| | | GGCTGGGTGCTGAGTTGGAATGAATATAGTATCTATCTCC | |
| | | TAGACACAGTCAACCAGGCCACAGTTGCTGGTTTGGAAGG | |
| | | ATCCGGTGATATTGTGTCTGTTTCGTGCACAGAAAATGAA | |
| | | ATATTTTTCTTGAAAGGAGATAGGAACATTATAAGAATTT | |
| | | CAAGCAGGCCTGAAGGATTAACATCAACAGTGAGAGATGG | |
| | | TCTGGAGATGTCTGGATGCTCAGAGCGTGTCCACGTGCAG | |
| | | CAAGCGGAGAAGCTGCCAGGGGCCACAGTTTCTGAGACGA | |
| | | GGCTCAGAGGCTCTTCCATGGCCAGCTCCGTGGCCAGCGA | |
| | | GCCAAGGAGCAGGAGCAGCTCGCTCAACTCCACCGACAGC | |
| | | GGCTCCGGGCTCCTGCCCCCTGGGCTCCAGGCCACCCCTG | |
| | | AGCTGGGCAAGGGCAGCCAGCCCCTGTCACAGAGATTCAA | |
| | | CGCCATCAGCTCAGAGGACTTTGACCAGGAGCTTGTCGTG | |
| | | AAGCCTATCAAAGTGAAAAGGAAGAAGAAGAAGAAGAAGA | |
| | | CAGAAGGTGGAAGCAGGAGCACCTGTCACAGCTCCCTGGA | |
| | | ATCGACACCCTGCTCCGAATTTCCTGGGGACAGTCCCCAG | |
| | | TCCTTGAACACAGACTTGCTGTCGATGACCTCAAGTGTCC | |
| | | TGGGCAGTAGCGTGGATCAGTTAAGTGCAGAGTCTCCAGA | |
| | | CCAGGAAAGCAGCTTCAATGGTGAAGTGAACGGTGTCCCA | |
| | | CAGGAAAATACTGACCCCGAAACGTTTAATGTCCTGGAGG | |
| | | TGTCAGGATCAATGCCTGATTCTCTGGCTGAGGAAGATGA | |
| | | CATTAGAACTGAAATGCCACACTGTCACCATGCACATGGG | |
| | | CGGGAGCTGCTCAATGGAGCGAGGGAAGATGTGGGAGGCA | |
| | | GTGATGTCACGGGACTCGGAGATGAGCCGTGTCCTGCAGA | |
| | | TGATGGACCAAATAGCACACAGTTACCCTTCCAAGAACAG | |
| | | GACAGCTCTCCTGGGGCGCATGATGGGGAAGACATCCAAC | |
| | | CCATTGGCCCCCAAAGCACTTTTTGTGAAGTCCCCCTCCT | |
| | | GAACTCACTCACTGTGCCTTCCAGCCTCAGCTGGGCCCCA | |
| | | AGTGCTGAACAGTGGCTGCCTGGGACCAGAGCTGATGAAG | |
| | | GCAGCCCCGTGGAGCCCAGCCAAGAGCAGGACATCCTAAC | |
| | | CAGCATGGAGGCCTCTGGCCACCTCAGCACAAATCTCTGG | |
| | | CATGCTGTCACTGATGATGACACAGGTCAGAAAGAAATAC | |
| | | CCATTTCTGAACGTGTCTTGGGGAGTGTGGGAGGACAGCT | |
| | | GACTCCGGTCTCTGCCTTGGCAGCCAGCACTCACAAGCCC | |
| | | TGGCTTGAGCAGCCTCCACGGGATCAGACATTGACGTCCA | |
| | | GCGATGAGGAGGACATCTATGCCCACGGGCTTCCTTCTTC | |
| | | ATCCTCAGAGACGAGTGTGACAGAGCTCGGACCTAGTTGC | |
| | | TCCCAGCAGGACCTGAGCCGGCTGGGTGCAGAGGACGCCG | |
| | | GGCTGCTCAAGCCAGATCAGTTTGCAGAAAGCTGGATGGG | |
| | | CTACTCGGGTCCCGGCTATGGCATCCTCAGCTTGGTGGTC | |
| | | TCCGAGAAGTATATCTGGTGCCTGGACTACAAAGGCGGCC | |
| | | TGTTCTGCAGCGCGTTGCCGGGCGCCGGGCTGCGCTGGCA | |
| | | GAAGTTTGAAGATGCTGTCCAGCAGGTGGCAGTCTCGCCC | |
| | | TCAGGAGCCCTTCTCTGGAAGATTGAACAGAAATCTAACC | |
| | | GGGCTTTTGCTTGTGGGAAAGTCACCATCAAGGGGAAGCG | |
| | | GCACTGGTACGAAGCCCTGCCCCAGGCAGTGTTTGTGGCC | |
| | | CTGAGCGATGACACGGCCTGGATCATCAGGACCAGTGGGG | |
| | | ACCTATACTTGCAGACAGGTCTGAGCGTGGATCGCCCTTG | |
| | | TGCCAGAGCCGTAAAGGTGGACTGTCCCTACCCGCTGTCC | |
| | | CAGATCACAGCCCGGAACAATGTGGTGTGGGCGCTGACAG | |
| | | AGCAGAGGGCCCTCCTGTACCGGGAGGGCGTGAGCAGCTT | |
| | | CTGTCCGGAAGGCGAGCAGTGGAAGTGTGACATTGTCAGC | |
| | | GAAAGGCAAGCTTTAGAACCCGTCTGCATAACGCTCGGGG | |
| | | ATCAGCAGACTCTCTGGGCCCTGGACATCCATGGGAACCT | |
| | | GTGGTTCAGAACTGGCATTATTTCCAAGAAGCCCCAAGGA | |
| | | GATGACGACCATTGGTGGCAAGTGAGCATCACGGACTATG | |
| | | TGGTGTTTGACCAGTGCAGCTTATTTCAGACGATAATCCA | |
| | | TGCCACTCACTCGGTGGCCACAGCAGCCCAAGCCCCCGTA | |
| | | GAAAAGGTGGCAGATAAGCTGCGCATGGCGTTTTGGTCCC | |
| | | AGCAGCTTCAGTGCCAGCCAAGCCTTCTCGGGGTCAATAA | |
| | | CAGCGGTGTCTGGATCTCCTCGGGCAAGAATGAATTCCAC | |
| | | GTCGCTAAGGGAAGTCTCATAGGCACCTACTGGAATCATG | |
| | | TGGTTCCCCGTGGGACAGCTTCTGCTACAAAATGGGCCTT | |
| | | TGTGTTGGCTTCTGCAGCTCCCACGAAGGAAGGAAGCTTC | |
| | | CTGTGGCTGTGCCAGAGCAGCAAGGACCTGTGCAGCGTCA | |
| | | GCGCCCAGAGCGCACAGTCGCGGCCCTCCACGGTGCAGCT | |
| | | GCCTCCCGAAGCCGAGATGCGCGCCTATGCCGCCTGCCAG | |
| | | GATGCGCTGTGGGCGCTGGACAGCCTCGGCCAGGTGTTCA | |
| | | TCAGGACGCTCTCCAAGAGCTGCCCCACGGGCATGCACTG | |
| | | GACCAGGCTGGACCTCTCCCAGCTAGGAGCTGTAAAATTG | |
| | | ACAAGCTTGGCATGTGGAAATCAGCACATCTGGGCCTGTG | |
| | | ATTCCAGGGGTGGAGTTTACTTCCGTGTAGGGACTCAGCC | |
| | | TCTCAATCCCAGTCTCATGCTTCCAGCCTGGATAATGATT | |
| | | GAGCCACCTGTCCAGGTAAGCAGAAGTTAGCTGGTGGAAC | |
| | | TCACTCTTCAGTAAGACAGAAACTGTGAGGATGCTGGTAC | |
| | | TGGGAAAAAGGATCTGCACAGCCTCTAGAGGCCTCCCAGC | |
| | | AAATGCGGGGAGCCATGCCCCCAGGGTCTACACACTCTCG | |
| | | TTCATCAACATCACAACTGGAATTCGGGATTTGTGAAGTT | |
| | | TAGAGCTGAACAGACTGTTACAGATTATGAGTCAACACGT | |
| | | ATATTTTCTCTTTCAAAATAATAATATTTCGTTTTTGACT | |
| | | TTTTACTAAGTGAATATTATTTTTTAAATCTGCCTATATA | |
| | | TTGGAACCTCTATTTTATAATAATAATGATAATAAATCAG | |
| | | TACCCAGAAGTATAAAGAAGGTAAAAGTTACTTTGAAAAA | |
| | | AAAAAAAAAAAAAAAAAAAAAAAAAAA | |
| TPH1 | NM_004179.2 | TTTTAGAGAATTACTCCAAATTCATCATGATTGAAGACAA | 44 |
| | | TAAGGAGAACAAAGACCATTCCTTAGAAAGGGGAAGAGCA | |
| | | AGTCTCATTTTTTCCTTAAAGAATGAAGTTGGAGGACTTA | |
| | | TAAAAGCCCTGAAAATCTTTCAGGAGAAGCATGTGAATCT | |
| | | GTTACATATCGAGTCCCGAAAATCAAAAAGAAGAAACTCA | |
| | | GAATTTGAGATTTTTGTTGACTGTGACATCAACAGAGAAC | |
| | | AATTGAATGATATTTTTCATCTGCTGAAGTCTCATACCAA | |
| | | TGTTCTCTCTGTGAATCTACCAGATAATTTTACTTTGAAG | |
| | | GAAGATGGTATGGAAACTGTTCCTTGGTTTCCAAAGAAGA | |
| | | TTTCTGACCTGGACCATTGTGCCAACAGAGTTCTGATGTA | |
| | | TGGATCTGAACTAGATGCAGACCATCCTGGCTTCAAAGAC | |
| | | AATGTCTACCGTAAACGTCGAAAGTATTTTGCGGACTTGG | |
| | | CTATGAACTATAAACATGGAGACCCCATTCCAAAGGTTGA | |
| | | ATTCACTGAAGAGGAGATTAAGACCTGGGGAACCGTATTC | |
| | | CAAGAGCTCAACAAACTCTACCCAACCCATGCTTGCAGAG | |
| | | AGTATCTCAAAAACTTACCTTTGCTTTCTAAATATTGTGG | |
| | | ATATCGGGAGGATAATATCCCACAATTGGAAGATGTCTCC | |
| | | AACTTTTTAAAAGAGCGTACAGGTTTTTCCATCCGTCCTG | |
| | | TGGCTGGTTACTTATCACCAAGAGATTTCTTATCAGGTTT | |
| | | AGCCTTTCGAGTTTTTCACTGCACTCAATATGTGAGACAC | |
| | | AGTTCAGATCCCTTCTATACCCCAGAGCCAGATACCTGCC | |
| | | ATGAACTCTTAGGTCATGTCCCGCTTTTGGCTGAACCTAG | |
| | | TTTTGCCCAATTCTCCCAAGAAATTGGCTTGGCTTCTCTT | |
| | | GGCGCTTCAGAGGAGGCTGTTCAAAAACTGGCAACGTGCT | |
| | | ACTTTTTCACTGTGGAGTTTGGTCTATGTAAACAAGATGG | |
| | | ACAGCTAAGAGTCTTTGGTGCTGGCTTACTTTCTTCTATC | |
| | | AGTGAACTCAAACATGCACTTTCTGGACATGCCAAAGTAA | |
| | | AGCCCTTTGATCCCAAGATTACCTGCAAACAGGAATGTCT | |
| | | TATCACAACTTTTCAAGATGTCTACTTTGTATCTGAAAGT | |
| | | TTTGAAGATGCAAAGGAGAAGATGAGAGAATTTACCAAAA | |
| | | CAATTAAGCGTCCATTTGGAGTGAAGTATAATCCATATAC | |
| | | ACGGAGTATTCAGATCCTGAAAGACACCAAGAGCATAACC | |
| | | AGTGCCATGAATGAGCTGCAGCATGATCTCGATGTTGTCA | |
| | | GTGATGCCCTTGCTAAGGTCAGCAGGAAGCCGAGTATCTA | |
| | | ACAGTAGCCAGTCATCCAGGAACATTTGAGCATCAATTCG | |
| | | GAGGTCTGGGCCATCTCTTGCTTTCCTTGAACACCTGATC | |
| | | CTGGAGGGACAGCATCTTCTGGCCAAACAATATTATCGAA | |
| | | TTCCACTACTTAAGGAATCACTAGTCTTTGAAAATTTGTA | |
| | | CCTGGATATTCTATTTACCACTTATTTTTTTGTTTAGTTT | |
| | | TATTTCTTTTTTTTTTTGGTAGCAGCTTTAATGAGACAAT | |
| | | TTATATACCATACAAGCCACTGACCACCCATTTTTAATAG | |
| | | AGAAGTTGTTTGACCCAATAGATAGATCTAATCTCAGCCT | |
| | | AACTCTATTTTCCCCAATCCTCCTTGAGTAAAATGACCCT | |
| | | TTAGGATCGCTTAGAATAACTTGAGGAGTATTATGGCGCT | |
| | | GACTCATATTGTTACCTAAGATCCCCTTATTTCTAAAGTA | |
| | | TCTGTTACTTATTGC | |
| TRMT112 | NM_016404.2 | GGCCACCCGCAGAACAGAGCTTCCGGGACCCACGCCTCGT | 45 |
| | | TTGCACTGGGTGCTGGACAGCCGACGCAACTACAAATGGG | |
| | | GCGGAGCTTTCGGCACTGGAGCAGCTAATTTGCATATAGG | |
| | | AATGAGGTGCGGCTCGGCTTCCATGGGCCTAATTTACAGA | |
| | | TAGGGCGGTATTTCTGCCCCTTAACCGAAAGTGGGATACA | |
| | | GAGGACGACGGTGTTAGGCGCCTGTGTAGGAGTAAAATGT | |
| | | GTTTATTTTGCATTCAACGAGAGCTCCTGCATTGCAGCTA | |
| | | TTTTGCATATGATTTGCATCTTACGAAGAATTTGTGGCAA | |
| | | AAAAAAGCTGGGCGTGCGCCGTAGGAACCTCCTGCTGAGA | |
| | | CGCTTCCGGTAGCGGCGCGTGACCCGACAGGTCTTTCACC | |
| | | TACCTACCTCAGCTCCCACAAACACGAGAAGTTCCAGCAA | |
| | | GTTCGCCACTTCCGGTTCTCCTGGCTATCCAATAGCATCG | |
| | | AGAGGAGCATCCCCGGAAGTGAGGCAGCGGAGGACGACCT | |
| | | TTTTCCGGTTCCGGCCTGGCGAGAGTTTGTGCGGCGACAT | |
| | | GAAACTGCTTACCCACAATCTGCTGAGCTCGCATGTGCGG | |
| | | GGGGTGGGGTCCCGTGGCTTCCCCCTGCGCCTCCAGGCCA | |
| | | CCGAGGTCCGTATCTGCCCTGTGGAATTCAACCCCAACTT | |
| | | CGTGGCGCGTATGATACCTAAAGTGGAGTGGTCGGCGTTC | |
| | | CTGGAGGCGGCCGATAACTTGCGTCTGATCCAGGTGCCGA | |
| | | AAGGGCCGGTTGAGGGATATGAGGAGAATGAGGAGTTTCT | |
| | | GAGGACCATGCACCACCTGCTGCTGGAGGTGGAAGTGATA | |
| | | GAGGGCACCCTGCAGTGCCCGGAATCTGGACGTATGTTCC | |
| | | CCATCAGCCGCGGGATCCCCAACATGCTGCTGAGTGAAGA | |
| | | GGAAACTGAGAGTTGATTGTGCCAGGCGCCAGTTTTTCTT | |
| | | GTTATGACTGTGTATTTTTGTTGATCTATACCCTGTTTCC | |
| | | GAATTCTGCCGTGTGTATCCCCAACCCTTGACCCAATGAC | |
| | | ACCAAACACAGTGTTTTTGAGCTCGGTATTATATATTTTT | |
| | | TTCTCATTAAAGGTTTAAAACCAAAAGCGGTTTCTCTTTG | |
| | | CAGCAAATATACATTAAAATAGAGTCTCTGTACAGCCAAG | |
| | | GGCTCTGGGCCCTGGCTTGCCCCATGTCCCTGCGCCTCCC | |
| | | TGGCCAAACCCAAAAATAAATATAGTGTTATTGCTCTGCA | |
| | | GGGCATAGAGGCAGTGCTCTCCTACCCCCTGAGGAGGCTC | |
| | | GTTGGGAGCTGATGGGGAAGCCCTG | |
| VMAT1 | NM_003053.3 | CACACACACACATACACAGAATCCTCAGATAACAGGAGGC | 46 |
| | | AATAAATCCAACAGCACATCCACGTTCAGAGAACAGTGTC | |
| | | CCTGCTGTCTTGCTAACAGCTGCCAATACCTCACTGAGTG | |
| | | CCTCACACCAACATGGGCTCCAAGTGAGTTTCCTTCGTCT | |
| | | GGGCAGACTCCCTCCCCTCTTCCATAAAGGCTGCAGGAGA | |
| | | CCTGTAGCTGTCACAGGACCTTCCCTAAGAGCCCGCAGGG | |
| | | GAAGACTGCCCCAGTCCGGCCATCACCATGCTCCGGACCA | |
| | | TTCTGGATGCTCCCCAGCGGTTGCTGAAGGAGGGGAGAGC | |
| | | GTCCCGGCAGCTGGTGCTGGTGGTGGTATTCGTCGCTTTG | |
| | | CTCCTGGACAACATGCTGTTTACTGTGGTGGTGCCAATTG | |
| | | TGCCCACCTTCCTATATGACATGGAGTTCAAAGAAGTCAA | |
| | | CTCTTCTCTGCACCTCGGCCATGCCGGAAGTTCCCCACAT | |
| | | GCCCTCGCCTCTCCTGCCTTTTCCACCATCTTCTCCTTCT | |
| | | TCAACAACAACACCGTGGCTGTTGAAGAAAGCGTACCTAG | |
| | | TGGAATAGCATGGATGAATGACACTGCCAGCACCATCCCA | |
| | | CCTCCAGCCACTGAAGCCATCTCAGCTCATAAAAACAACT | |
| | | GCTTGCAAGGCACAGGTTTCTTGGAGGAAGAGATTACCCG | |
| | | GGTCGGGGTTCTGTTTGCTTCAAAGGCTGTGATGCAACTT | |
| | | CTGGTCAACCCATTCGTGGGCCCTCTCACCAACAGGATTG | |
| | | GATATCATATCCCCATGTTTGCTGGCTTTGTTATCATGTT | |
| | | TCTCTCCACAGTTATGTTTGCTTTTTCTGGGACCTATACT | |
| | | CTACTCTTTGTGGCCCGAACCCTTCAAGGCATTGGATCTT | |
| | | CATTTTCATCTGTTGCAGGTCTTGGAATGCTGGCCAGTGT | |
| | | CTACACTGATGACCATGAGAGAGGACGAGCCATGGGAACT | |
| | | GCTCTGGGGGGCCTGGCCTTGGGGTTGCTGGTGGGAGCTC | |
| | | CCTTTGGAAGTGTAATGTACGAGTTTGTTGGGAAGTCTGC | |
| | | ACCCTTCCTCATCCTGGCCTTCCTGGCACTACTGGATGGA | |
| | | GCACTCCAGCTTTGCATCCTACAGCCTTCCAAAGTCTCTC | |
| | | CTGAGAGTGCCAAGGGGACTCCCCTCTTTATGCTTCTCAA | |
| | | AGACCCTTACATCCTGGTGGCTGCAGGGTCCATCTGCTTT | |
| | | GCCAACATGGGGGTGGCCATCCTGGAGCCCACACTGCCCA | |
| | | TCTGGATGATGCAGACCATGTGCTCCCCCAAGTGGCAGCT | |
| | | GGGTCTAGCTTTCTTGCCTGCCAGTGTGTCCTACCTCATT | |
| | | GGCACCAACCTCTTTGGTGTGTTGGCCAACAAGATGGGTC | |
| | | GGTGGCTGTGTTCCCTAATCGGGATGCTGGTAGTAGGTAC | |
| | | CAGCTTGCTCTGTGTTCCTCTGGCTCACAATATTTTTGGT | |
| | | CTCATTGGCCCCAATGCAGGGCTTGGCCTTGCCATAGGCA | |
| | | TGGTGGATTCTTCTATGATGCCCATCATGGGGCACCTGGT | |
| | | GGATCTACGCCACACCTCGGTGTATGGGAGTGTCTACGCC | |
| | | ATCGCTGATGTGGCTTTTTGCATGGGCTTTGCTATAGGTC | |
| | | CATCCACCGGTGGTGCCATTGTAAAGGCCATCGGTTTTCC | |
| | | CTGGCTCATGGTCATCACTGGGGTCATCAACATCGTCTAT | |
| | | GCTCCACTCTGCTACTACCTGCGGAGCCCCCCGGCAAAGG | |
| | | AAGAGAAGCTTGCTATTCTGAGTCAGGACTGCCCCATGGA | |
| | | GACCCGGATGTATGCAACCCAGAAGCCCACGAAGGAATTT | |
| | | CCTCTGGGGGAGGACAGTGATGAGGAGCCTGACCATGAGG | |
| | | AGTAGCAGCAGAAGGTGCTCCTTGAATTCATGATGCCTCA | |
| | | GTGACCACCTCTTTCCCTGGGACCAGATCACCATGGCTGA | |
| | | GCCCACGGCTCAGTGGGCTTCACATACCTCTGCCTGGGAA | |
| | | TCTTCTTTCCTCCCCTCCCATGGACACTGTCCCTGATACT | |
| | | CTTCTCACCTGTGTAACTTGTAGCTCTTCCTCTATGCCTT | |
| | | GGTGCCGCAGTGGCCCATCTTTTATGGGAAGACAGAGTGA | |
| | | TGCACCTTCCCGCTGCTGTGAGGTTGATTAAACTTGAGCT | |
| | | GTGACGGGTTCTGCAAGGGGTGACTCATTGCATAGAGGTG | |
| | | GTAGTGAGTAATGTGCCCCTGAAACCAGTGGGGTGACTGA | |
| | | CAAGCCTCTTTAATCTGTTGCCTGATTTTCTCTGGCATAG | |
| | | TCCCAACAGATCGGAAGAGTGTTACCCTCTTTTCCTCAAC | |
| | | GTGTTCTTTCCCGGGTTTTCCCAGCCGAGTTGAGAAAATG | |
| | | TTCTCAGCATTGTCTTGCTGCCAAATGCCAGCTTGAAGAG | |
| | | TTTTGTTTTGTTTTTTTTCATTTATTTTTTTTTTTAATAA | |
| | | AGTGAGTGATTTTTCTGTGGCTAAATCTAGAGCTGCTAAA | |
| | | AGGGCTTTACCCTCAGTGAAAAGTGTCTTCTATTTTCATT | |
| | | ATCTTTCAGAAACAGGAGCCCATTTCTCTTCTGCTGGAGT | |
| | | TATTGACATTCTCCTGACCTCCCCTGTGTGTTCCTACCTT | |
| | | TTCTGAACCTCTTAGACTCTTAGAAATAAAAGTAGAAGAA | |
| | | AGACAGAAAAAATAACTGATTAGACCCAAGATTTCATGGG | |
| | | AAGAAGTTAAAAGAAACTGCCTTGAAATCCCTCCTGATTG | |
| | | TAGATTTCCTAACAGGAGGGGTGTAATGTGACATTGTTCA | |
| | | TACTTGCTAATAAATACATTATTGCCTAATTCAAAAAAAA | |
| | | AAAAAAAAAA | |
| VMAT2 | NM_003054.4 | AGAGCCGGACGGGGTAAACTGAGCGGCGGCGGCGGGGCGC | 47 |
| | | TGGGGCGGAGACTGCGACCCGGAGCCGCCCGGACTGACGG | |
| | | AGCCCACTGCGGTGCGGGCGTTGGCGCGGGCACGGAGGAC | |
| | | CCGGGCAGGCATCGCAAGCGACCCCGAGCGGAGCCCCGGA | |
| | | GCCATGGCCCTGAGCGAGCTGGCGCTGGTCCGCTGGCTGC | |
| | | AGGAGAGCCGCCGCTCGCGGAAGCTCATCCTGTTCATCGT | |
| | | GTTCCTGGCGCTGCTGCTGGACAACATGCTGCTCACTGTC | |
| | | GTGGTCCCCATCATCCCAAGTTATCTGTACAGCATTAAGC | |
| | | ATGAGAAGAATGCTACAGAAATCCAGACGGCCAGGCCAGT | |
| | | GCACACTGCCTCCATCTCAGACAGCTTCCAGAGCATCTTC | |
| | | TCCTATTATGATAACTCGACTATGGTCACCGGGAATGCTA | |
| | | CCAGAGACCTGACACTTCATCAGACCGCCACACAGCACAT | |
| | | GGTGACCAACGCGTCCGCTGTTCCTTCCGACTGTCCCAGT | |
| | | GAAGACAAAGACCTCCTGAATGAAAACGTGCAAGTTGGTC | |
| | | TGTTGTTTGCCTCGAAAGCCACCGTCCAGCTCATCACCAA | |
| | | CCCTTTCATAGGACTACTGACCAACAGAATTGGCTATCCA | |
| | | ATTCCCATATTTGCGGGATTCTGCATCATGTTTGTCTCAA | |
| | | CAATTATGTTTGCCTTCTCCAGCAGCTATGCCTTCCTGCT | |
| | | GATTGCCAGGTCGCTGCAGGGCATCGGCTCGTCCTGCTCC | |
| | | TCTGTGGCTGGGATGGGCATGCTTGCCAGTGTCTACACAG | |
| | | ATGATGAAGAGAGAGGCAACGTCATGGGAATCGCCTTGGG | |
| | | AGGCCTGGCCATGGGGGTCTTAGTGGGCCCCCCCTTCGGG | |
| | | AGTGTGCTCTATGAGTTTGTGGGGAAGACGGCTCCGTTCC | |
| | | TGGTGCTGGCCGCCCTGGTACTCTTGGATGGAGCTATTCA | |
| | | GCTCTTTGTGCTCCAGCCGTCCCGGGTGCAGCCAGAGAGT | |
| | | CAGAAGGGGACACCCCTAACCACGCTGCTGAAGGACCCGT | |
| | | ACATCCTCATTGCTGCAGGCTCCATCTGCTTTGCAAACAT | |
| | | GGGCATCGCCATGCTGGAGCCAGCCCTGCCCATCTGGATG | |
| | | ATGGAGACCATGTGTTCCCGAAAGTGGCAGCTGGGCGTTG | |
| | | CCTTCTTGCCAGCTAGTATCTCTTATCTCATTGGAACCAA | |
| | | TATTTTTGGGATACTTGCACACAAAATGGGGAGGTGGCTT | |
| | | TGTGCTCTTCTGGGAATGATAATTGTTGGAGTCAGCATTT | |
| | | TATGTATTCCATTTGCAAAAAACATTTATGGACTCATAGC | |
| | | TCCGAACTTTGGAGTTGGTTTTGCAATTGGAATGGTGGAT | |
| | | TCGTCAATGATGCCTATCATGGGCTACCTCGTAGACCTGC | |
| | | GGCACGTGTCCGTCTATGGGAGTGTGTACGCCATTGCGGA | |
| | | TGTGGCATTTTGTATGGGGTATGCTATAGGTCCTTCTGCT | |
| | | GGTGGTGCTATTGCAAAGGCAATTGGATTTCCATGGCTCA | |
| | | TGACAATTATTGGGATAATTGATATTCTTTTTGCCCCTCT | |
| | | CTGCTTTTTTCTTCGAAGTCCACCTGCCAAAGAAGAAAAA | |
| | | ATGGCTATTCTCATGGATCACAACTGCCCTATTAAAACAA | |
| | | AAATGTACACTCAGAATAATATCCAGTCATATCCGATAGG | |
| | | TGAAGATGAAGAATCTGAAAGTGACTGAGATGAGATCCTC | |
| | | AAAAATCATCAAAGTGTTTAATTGTATAAAACAGTGTTTC | |
| | | CAGTGACACAACTCATCCAGAACTGTCTTAGTCATACCAT | |
| | | CCATCCCTGGTGAAAGAGTAAAACCAAAGGTTATTATTTC | |
| | | CTTTCCATGGTTATGGTCGATTGCCAACAGCCTTATAAAG | |
| | | AAAAAGAAGCTTTTCTAGGGGTTTGTATAAATAGTGTTGA | |
| | | AACTTTATTTTATGTATTTAATTTTATTAAATATCATACA | |
| | | ATATATTTTGATGAAATAGGTATTGTGTAAATCTATAAAT | |
| | | ATTTGAATCCAAACCAAATATAATTTTTTAACTTACATTA | |
| | | ACAAACATTTGGGCAAAAATCATATTGGTAATGAGTGTTT | |
| | | AAAATTAAAGCACACATTATCTCTGAGACTCTTCCAACAA | |
| | | AGAGAAACTAGAATGAAGTCTGAAAAACAGAATCAAGTAA | |
| | | GACAGCATGTTATATAGTGACACTGAATGTTATTTAACTT | |
| | | GTAGTTACTATCAATATATTTATGCGTTAAACAGCTAGTT | |
| | | CTCTCAAGTGTAGAGGACAAGAACTTGTGTCAGTTATCTT | |
| | | TTGAATCCATAAATCTTAGCTGGCATTAGTTTTCTATGTA | |
| | | ATCACCTACCTAGAGAGAGTTGTAAATTATATGTTAACAT | |
| | | GTTATCTGGTTGGCAGCAAACACTAAAGCCAATAAAGGAA | |
| | | AAACAGTAAATGTTCCGAAAGCAGAGAAAAGCAACCAAAC | |
| | | ATATTGTTATGAACTAAAAGCTTTCCCTTTAAGATGCATA | |
| | | CTTGTCTTACTGGATGAAGAAAATTGAGGGTACATGTACC | |
| | | TTATACTGTCAAGGTTGTTTAAACATGATAAGGTTAATCG | |
| | | CCATCTACTTCAAGTTTTAGAAAAGGAAACAAGAAGCTGA | |
| | | AAACAGCTGCTCTGACTTTAATATCTGACTATATCTTTGA | |
| | | TCTGTTTGCAGGTCATCCAAGTGTTTTCTAGGAATATATT | |
| | | TATTTTAGGTTGTCTGAAACTACTATTTTTTAGACTCCTG | |
| | | AAAGTTGTTCACATCAATGTGAAGACAAATTTTAAATGAA | |
| | | AATGAAGAATGAAATTATGTCTTGAATCATATATTAAGAA | |
| | | GTAAAAATAATAGTGATCAGGCAGAAAAGAAAAATGGAAC | |
| | | ATCTAAAAATGTATGTGCTAACTATATCATCCAGTGTGCA | |
| | | GTGTTGTGTATTTTTCTAAGCATGACAACATTGATGTGCC | |
| | | TTTTCAGTGTAACAGCAAATACTGTTAGTGAACATTGTCA | |
| | | ATTTATGTCATTTTGTTAAGAGATATGACTGGAGTGTGCA | |
| | | GTGTGGAATGTCTCTAATACTACTTGTGAATCCTGCAGTT | |
| | | CTATAATCATAAACAAAAATTACTTAGTTTCGTTAAGCTA | |
| | | AGATTGTGTTTGTGTTAACTTCGACATCAAGGAGCAAAGA | |
| | | ACTTTAGAACAGACTCCTCAATCTTGTGACTTTCTTATTC | |
| | | TCTAGGAAAGTAACACTTCGTTTCATGAAGCTTTTCTGTG | |
| | | GGGCTTCGATTATTTCAAGTCTGGTTTCTAAGTGCAGTGT | |
| | | GTTTGAAGCAAACGAACTTCCAACTCACTTATTTGGCATT | |
| | | GGGCAACTTGGCCAAGTCTGCCACTTTGGAAGATGGCTCT | |
| | | GGAGGAAACTCTCATATGGCTAAAAAGGCAGGCTAGTTTC | |
| | | TTACTTCTACAGGGGTAGAGCCTTAAAAAAGAACGTGCTA | |
| | | CAAATTGGTTCTCTTTGAGGGTTTCTGGTTCTCCCTGCCC | |
| | | CCAATACCATATACTTTATTGCAATTTTATTTTTGCCTTT | |
| | | ACGGCTCTGTGTCTTTCTGCAAGAAGGCCTGGCAAAGGTA | |
| | | TGCCTGCTGTTGGTCCCTCGGGATAAGATAAAATATAAAT | |
| | | AAAACCTTCAGAACTGTTTTGGAGCAAAAGATAGCTTGTA | |
| | | CTTGGGGAAAAAAATTCTAAGTTCTTTTATATGACTAATA | |
| | | TTCTTGGTTAGCAAGACTGGAAAGAGGTGTTTTTTTAAAA | |
| | | TGTACATACCAGAACAAAGAACATACAGCTCTCTGAACAT | |
| | | TTATTTTTTGAACAGAGGTGGTTTTTATGTTTGGACCTGG | |
| | | TAATACAGATACAAAAACTTTAATGAGGTAGCAATGAATA | |
| | | TTCAACTGTTTGACTGCTAAGTGTATCTGTCCATATTTTA | |
| | | GCAAGTTTACTTAATAAATCTTCTGAACCATGAAAAAAAA | |
| | | AAAAA | |
| VPS13C | NM_001018088.2 | CCGGAGGGGCTGTCATTTGCAGCGCTGGTCGCAGCCCTCA | 48 |
| | | GCTGCGCCGGGCGGTTCCGGCTCCTCCCTCTCCTTGTGCC | |
| | | TCAGCGCCACCATGGTGCTGGAGTCGGTGGTCGCGGACTT | |
| | | GCTGAACCGCTTCCTGGGGGACTATGTGGAGAACCTGAAC | |
| | | AAGTCCCAGCTGAAGCTGGGCATCTGGGGCGGAAATGTGG | |
| | | CTTTAGATAATCTACAGATAAAAGAAAATGCCCTGAGTGA | |
| | | ATTGGATGTTCCTTTTAAAGTCAAGGCTGGCCAAATTGAT | |
| | | AAATTAACTTTGAAGATTCCTTGGAAGAACCTTTATGGAG | |
| | | AAGCAGTTGTTGCGACCCTGGAAGGATTATACCTGCTTGT | |
| | | TGTCCCTGGAGCAAGTATTAAGTATGATGCTGTAAAAGAA | |
| | | GAAAAATCCTTGCAGGATGTTAAACAGAAAGAGCTATCCC | |
| | | GAATTGAAGAAGCCCTTCAAAAAGCAGCAGAAAAAGGCAC | |
| | | ACATTCAGGGGAGTTCATATATGGCTTGGAGAACTTTGTT | |
| | | TACAAGGACATCAAGCCTGGACGTAAACGTAAAAAGCACA | |
| | | AAAAACATTTTAAGAAACCTTTTAAAGGTCTTGATCGTTC | |
| | | AAAAGATAAGCCAAAAGAAGCCAAAAAGGATACATTTGTG | |
| | | GAAAAATTGGCAACTCAAGTAATAAAAAATGTACAAGTAA | |
| | | AAATCACAGATATTCACATTAAATATGAAGATGATGTCAC | |
| | | TGATCCAAAGCGGCCTCTTTCATTTGGTGTCACACTGGGA | |
| | | GAGCTTAGTCTACTGACTGCAAATGAACACTGGACTCCAT | |
| | | GCATATTAAATGAAGCAGACAAAATTATATACAAGCTTAT | |
| | | ACGACTTGATAGTCTTAGCGCCTACTGGAATGTAAATTGC | |
| | | AGCATGTCTTACCAGAGATCAAGGGAACAGATTTTGGATC | |
| | | AGCTGAAAAATGAAATTCTTACAAGTGGAAATATACCCCC | |
| | | AAATTATCAATACATTTTCCAGCCAATATCAGCCTCTGCA | |
| | | AAACTCTACATGAATCCTTATGCAGAATCAGAGCTCAAAA | |
| | | CGCCCAAACTGGATTGCAACATAGAAATACAAAATATTGC | |
| | | CATTGAACTGACCAAACCTCAGTACTTAAGTATGATTGAC | |
| | | CTTTTGGAGTCAGTGGATTATATGGTTAGGAATGCGCCTT | |
| | | ATAGGAAATACAAGCCTTATTTACCACTTCATACCAATGG | |
| | | TCGACGATGGTGGAAATATGCAATTGATTCTGTTCTTGAA | |
| | | GTTCATATAAGAAGGTATACACAGATGTGGTCATGGAGTA | |
| | | ACATAAAAAAGCACAGGCAGTTACTCAAGAGTTATAAAAT | |
| | | TGCCTACAAAAACAAGTTAACACAGTCTAAAGTCTCAGAA | |
| | | GAAATACAGAAAGAAATTCAGGACTTGGAGAAGACTCTAG | |
| | | ATGTTTTTAACATAATTTTAGCAAGGCAACAAGCACAAGT | |
| | | TGAGGTGATTCGGTCTGGGCAAAAATTAAGGAAAAAGTCT | |
| | | GCTGACACAGGCGAGAAACGTGGAGGCTGGTTTAGTGGGT | |
| | | TGTGGGGTAAGAAAGAGTCTAAGAAAAAGGACGAAGAATC | |
| | | ATTGATTCCTGAAACTATTGATGACCTTATGACTCCAGAG | |
| | | GAAAAAGATAAACTCTTCACTGCCATTGGTTATAGTGAGA | |
| | | GTACCCACAACCTAACTTTACCTAAGCAGTATGTTGCCCA | |
| | | TATTATGACCCTGAAGTTAGTAAGCACCTCTGTTACGATA | |
| | | AGAGAAAACAAGAATATTCCAGAAATACTAAAAATTCAGA | |
| | | TAATTGGCCTGGGCACTCAAGTATCTCAGCGACCAGGAGC | |
| | | ACAAGCACTTAAGGTAGAAGCGAAATTAGAACACTGGTAT | |
| | | ATAACAGGTTTGAGACAGCAGGATATTGTGCCATCACTTG | |
| | | TGGCTTCAATTGGTGACACTACATCATCCTTGCTTAAAAT | |
| | | TAAATTTGAAACCAATCCGGAGGATAGTCCTGCTGACCAG | |
| | | ACTCTGATTGTTCAGTCCCAGCCTGTGGAGGTCATCTATG | |
| | | ATGCTAAAACTGTCAATGCAGTGGTTGAATTCTTTCAATC | |
| | | AAATAAGGGATTGGATCTTGAGCAAATAACATCAGCAACA | |
| | | TTGATGAAGCTGGAAGAAATTAAGGAGAGAACAGCTACAG | |
| | | GACTTACACATATTATTGAAACTCGAAAAGTCCTTGATTT | |
| | | AAGGATAAATCTGAAGCCTTCTTATCTAGTAGTTCCACAG | |
| | | ACGGGTTTCCACCATGAAAAGTCAGATCTTCTGATTTTAG | |
| | | ATTTTGGTACATTTCAGCTCAACAGTAAAGATCAAGGTTT | |
| | | ACAGAAGACTACTAATTCATCTCTGGAAGAAATAATGGAT | |
| | | AAGGCATATGACAAGTTTGATGTTGAAATAAAAAATGTAC | |
| | | AACTACTTTTTGCAAGAGCAGAGGAAACCTGGAAAAAGTG | |
| | | TCGATTTCAGCATCCATCAACTATGCATATATTGCAACCC | |
| | | ATGGATATTCATGTTGAGTTGGCTAAGGCCATGGTAGAAA | |
| | | AAGACATTAGAATGGCCAGATTTAAAGTGTCAGGAGGACT | |
| | | TCCTTTGATGCATGTGAGAATTTCTGACCAGAAGATGAAA | |
| | | GATGTGCTATATTTGATGAACAGTATACCTTTGCCACAGA | |
| | | AATCATCAGCCCAGTCTCCAGAGAGACAGGTATCCTCAAT | |
| | | TCCTATTATTTCAGGTGGTACAAAAGGTCTACTTGGTACT | |
| | | TCACTATTGCTAGACACTGTGGAATCAGAGTCTGATGATG | |
| | | AGTATTTTGATGCTGAAGATGGAGAACCACAGACTTGTAA | |
| | | AAGTATGAAAGGATCAGAACTTAAAAAAGCTGCAGAGGTC | |
| | | CCAAATGAGGAGCTCATCAATCTTCTACTCAAGTTTGAAA | |
| | | TTAAAGAAGTGATTTTGGAATTTACTAAACAGCAGAAAGA | |
| | | AGAAGATACAATTCTAGTATTTAATGTTACTCAGTTAGGA | |
| | | ACAGAGGCCACAATGAGAACATTTGACTTAACTGTGGTAT | |
| | | CTTATTTAAAGAAAATCAGCTTGGATTATCATGAAATTGA | |
| | | AGGATCCAAAAGGAAGCCCCTTCACTTGATTAGCTCTTCT | |
| | | GACAAACCTGGATTAGATCTTTTGAAAGTGGAGTATATTA | |
| | | AGGCTGATAAGAATGGACCTAGTTTTCAAACTGCTTTTGG | |
| | | AAAAACTGAACAAACAGTTAAGGTGGCCTTTTCATCTTTA | |
| | | AATCTGTTGCTGCAAACACAAGCTCTTGTCGCTTCTATTA | |
| | | ATTACCTCACAACCATTATTCCATCTGATGATCAAAGCAT | |
| | | AAGTGTTGCTAAGGAGGTACAAATTTCAACTGAAAAACAA | |
| | | CAAAAAAATTCAACTCTGCCAAAAGCGATTGTATCCTCCA | |
| | | GAGATAGTGACATTATTGATTTCAGGCTATTTGCCAAGTT | |
| | | GAATGCTTTCTGTGTCATTGTTTGCAACGAAAAGAACAAT | |
| | | ATCGCCGAAATCAAGATTCAAGGACTGGATTCCTCCCTTT | |
| | | CTCTCCAGTCAAGAAAGCAGTCACTTTTTGCCCGACTAGA | |
| | | AAATATTATTGTCACAGATGTTGATCCAAAGACAGTTCAT | |
| | | AAGAAAGCTGTGTCAATAATGGGAAATGAAGTTTTCCGTT | |
| | | TTAATTTGGATTTGTATCCAGATGCTACTGAGGGGGATTT | |
| | | GTATACTGACATGTCCAAAGTGGATGGTGTGCTGTCTCTG | |
| | | AATGTTGGCTGTATTCAGATTGTCTATCTTCATAAATTCC | |
| | | TTATGTCACTTCTGAACTTCCTGAATAATTTCCAGACAGC | |
| | | CAAAGAGTCTCTGAGTGCTGCCACTGCCCAGGCTGCAGAA | |
| | | AGGGCTGCCACAAGTGTGAAAGATCTTGCCCAGAGGAGTT | |
| | | TTCGTGTTTCCATCAATATTGATTTGAAAGCACCGGTTAT | |
| | | AGTCATCCCACAGTCTTCTATTTCCACCAATGCAGTAGTG | |
| | | GTAGATCTTGGGTTAATCAGAGTTCATAATCAGTTCAGTC | |
| | | TGGTGTCTGATGAAGACTACTTAAATCCTCCAGTAATTGA | |
| | | TAGAATGGATGTGCAGCTAACAAAGCTTACACTTTATAGG | |
| | | ACAGTGATCCAGCCAGGCATCTACCATCCTGATATTCAGC | |
| | | TGTTGCACCCAATTAACTTGGAATTTCTTGTAAATCGGAA | |
| | | TCTAGCTGCATCTTGGTACCACAAGGTGCCTGTTGTGGAA | |
| | | ATTAAAGGACATCTTGATTCAATGAATGTTAGTCTAAATC | |
| | | AAGAAGATCTTAATCTTTTATTTAGGATACTAACAGAAAA | |
| | | TCTCTGTGAGGGTACTGAAGACTTGGATAAAGTGAAACCA | |
| | | AGAGTACAAGAGACAGGTGAAATTAAAGAGCCCCTTGAAA | |
| | | TCTCTATATCACAAGATGTACATGATTCAAAAAATACTTT | |
| | | AACAACTGGAGTGGAAGAAATTAGGTCTGTAGACATCATT | |
| | | AATATGCTGCTGAATTTTGAAATTAAAGAGGTTGTGGTTA | |
| | | CTTTGATGAAAAAATCAGAAAAGAAAGGAAGGCCTTTACA | |
| | | TGAGCTAAATGTCCTGCAACTTGGAATGGAAGCTAAAGTT | |
| | | AAAACCTATGACATGACTGCTAAAGCTTATCTAAAAAAAA | |
| | | TTAGTATGCAGTGCTTTGATTTCACTGACTCTAAAGGGGA | |
| | | ACCTCTTCACATTATTAACTCTTCTAATGTGACTGACGAA | |
| | | CCCCTTCTGAAAATGTTACTGACAAAGGCAGACAGTGATG | |
| | | GACCAGAATTTAAAACTATTCATGACAGTACCAAACAGAG | |
| | | ACTGAAGGTTTCATTTGCATCCTTAGACTTAGTACTTCAT | |
| | | TTGGAAGCTTTACTTTCCTTCATGGATTTTTTATCATCTG | |
| | | CTGCTCCATTCTCTGAGCCTTCCTCTTCTGAGAAGGAATC | |
| | | CGAGCTGAAACCACTTGTGGGGGAGTCCAGAAGTATCGCT | |
| | | GTCAAAGCTGTATCCAGCAACATTTCCCAAAAGGATGTGT | |
| | | TTGATTTAAAGATCACAGCTGAATTAAATGCATTTAATGT | |
| | | CTTTGTCTGTGATCAGAAGTGTAACATTGCAGATATTAAA | |
| | | ATACATGGAATGGATGCCTCTATTTCTGTGAAGCCTAAGC | |
| | | AGACTGATGTGTTTGCCAGACTTAAAGATATTATAGTTAT | |
| | | GAATGTAGATTTGCAGTCCATTCACAAAAAGGCTGTCTCT | |
| | | ATTTTGGGAGATGAAGTCTTTAGGTTCCAACTGACTCTTT | |
| | | ATCCAGATGCCACAGAAGGAGAGGCCTATGCTGATATGTC | |
| | | CAAAGTAGACGGCAAACTTAGTTTTAAAGTGGGTTGTATT | |
| | | CAGATTGTTTATGTTCATAAATTCTTCATGTCTCTTTTGA | |
| | | ACTTCCTCAACAATTTCCAAACTGCTAAAGAAGCTTTGAG | |
| | | TACAGCCACAGTCCAGGCTGCAGAAAGAGCTGCTTCCAGC | |
| | | ATGAAAGACTTGGCTCAAAAGAGTTTCCGCCTTTTGATGG | |
| | | ATATTAATTTGAAAGCACCAGTTATTATTATTCCTCAGTC | |
| | | TTCAGTATCACCTAATGCTGTTATAGCAGATCTGGGTTTA | |
| | | ATCAGAGTTGAAAACAAGTTTAGCTTGGTTCCTATGGAAC | |
| | | ATTATTCTCTTCCTCCAGTCATTGATAAAATGAACATCGA | |
| | | ACTCACTCAGTTGAAGCTGTCAAGAACTATTTTGCAGGCT | |
| | | AGCTTGCCACAAAATGACATTGAAATTTTAAAACCAGTCA | |
| | | ACATGCTTTTGTCCATACAGCGAAACTTAGCAGCAGCATG | |
| | | GTATGTGCAAATTCCAGGGATGGAGATAAAAGGAAAACTA | |
| | | AAACCTATGCAGGTTGCTCTCAGTGAAGATGACTTGACAG | |
| | | TTTTAATGAAAATTTTGCTAGAAAATCTTGGAGAAGCTTC | |
| | | CTCACAACCAAGCCCTACACAGTCTGTGCAGGAGACTGTA | |
| | | AGAGTGAGAAAAGTTGATGTTTCAAGTGTACCTGACCATC | |
| | | TCAAAGAACAAGAAGATTGGACAGACTCAAAGCTCTCTAT | |
| | | GAACCAGATTGTCAGTCTCCAATTTGACTTTCACTTTGAA | |
| | | TCTCTTTCCATTATCCTTTATAACAATGATATCAACCAGG | |
| | | AATCTGGAGTTGCATTTCATAATGACAGTTTCCAACTTGG | |
| | | TGAACTCAGACTACATCTTATGGCCTCCTCAGGGAAGATG | |
| | | TTTAAGGATGGCTCAATGAATGTCAGCGTTAAACTTAAGA | |
| | | CATGCACCCTTGATGATCTCAGAGAAGGAATTGAGAGAGC | |
| | | AACATCGAGAATGATTGACAGAAAGAATGACCAAGATAAC | |
| | | AACAGTTCTATGATTGATATAAGTTACAAACAAGACAAAA | |
| | | ATGGAAGTCAAATTGATGCTGTTCTTGACAAGCTGTATGT | |
| | | ATGTGCCAGTGTGGAATTTCTGATGACTGTGGCAGATTTC | |
| | | TTTATCAAAGCTGTGCCTCAGAGTCCAGAAAATGTGGCAA | |
| | | AAGAAACACAGATTTTACCAAGACAGACTGCCACAGGGAA | |
| | | GGTCAAGATAGAGAAAGATGACTCTGTTAGACCAAATATG | |
| | | ACTTTAAAGGCCATGATCACAGATCCAGAAGTGGTATTTG | |
| | | TTGCCAGCCTGACAAAGGCTGATGCTCCTGCTCTGACAGC | |
| | | CTCGTTTCAGTGCAACCTTTCTCTGTCAACATCCAAACTC | |
| | | GAACAGATGATGGAAGCTTCTGTGAGAGATCTGAAAGTGC | |
| | | TCGCTTGCCCTTTTCTCAGAGAAAAGAGAGGGAAAAACAT | |
| | | TACCACAGTCTTGCAGCCCTGTTCTTTATTTATGGAAAAA | |
| | | TGTACGTGGGCTTCAGGAAAGCAAAATATAAATATTATGG | |
| | | TTAAAGAATTTATAATTAAGATTTCACCCATAATTCTTAA | |
| | | TACTGTGTTGACAATCATGGCTGCATTGTCTCCAAAAACA | |
| | | AAAGAAGATGGATCCAAAGATACGTCTAAGGAAATGGAAA | |
| | | ATCTTTGGGGTATCAAATCGATTAATGATTATAACACTTG | |
| | | GTTTCTTGGTGTTGACACGGCAACAGAAATAACGGAAAGC | |
| | | TTCAAAGGCATTGAACATTCACTGATAGAGGAAAATTGTG | |
| | | GTGTTGTTGTAGAATCCATTCAAGTTACCTTAGAATGTGG | |
| | | CCTTGGACATCGAACTGTACCTTTATTATTGGCAGAGTCT | |
| | | AAGTTTTCAGGAAATATTAAAAATTGGACTTCTCTAATGG | |
| | | CTGCTGTTGCTGACGTGACACTACAGGTGCACTATTACAA | |
| | | TGAGATCCATGCTGTCTGGGAGCCACTGATTGAGAGAGTG | |
| | | GAGGGGAAGAGACAATGGAATTTAAGGCTTGATGTAAAGA | |
| | | AGAACCCAGTTCAGGATAAAAGTTTGCTGCCAGGAGATGA | |
| | | TTTTATTCCTGAGCCACAAATGGCAATTCATATTTCTTCA | |
| | | GGAAATACAATGAATATAACAATATCCAAAAGTTGTCTTA | |
| | | ATGTTTTCAACAATTTAGCAAAAGGTTTTTCAGAGGGCAC | |
| | | TGCTTCTACTTTTGACTACTCTTTAAAGGACAGAGCTCCT | |
| | | TTTACGGTAAAAAATGCTGTAGGTGTTCCCATTAAGGTGA | |
| | | AGCCCAATTGTAATCTCAGAGTAATGGGCTTCCCTGAGAA | |
| | | AAGTGATATTTTTGATGTTGATGCTGGCCAGAATTTGGAA | |
| | | CTGGAGTATGCCAGCATGGTACCTTCAAGTCAAGGGAACC | |
| | | TATCTATATTGAGCCGTCAAGAAAGCTCCTTCTTCACTCT | |
| | | GACCATTGTACCTCATGGATATACAGAAGTTGCAAATATC | |
| | | CCTGTGGCCAGACCTGGACGGCGATTGTATAATGTACGGA | |
| | | ATCCCAATGCCAGTCATTCTGACTCTGTCTTGGTACAAAT | |
| | | TGATGCAACTGAAGGGAATAAAGTAATTACCCTTCGCTCT | |
| | | CCTCTACAGATCAAAAACCATTTCTCCATTGCATTTATCA | |
| | | TCTATAAATTTGTTAAGAATGTTAAGCTATTGGAGCGCAT | |
| | | TGGGATAGCCAGACCTGAAGAGGAGTTCCATGTTCCTTTA | |
| | | GATTCATATAGATGTCAATTGTTTATCCAGCCAGCTGGAA | |
| | | TCTTAGAGCATCAGTACAAAGAATCTACCACTTATATTTC | |
| | | CTGGAAGGAAGAACTTCATAGGAGCAGGGAAGTCAGATGC | |
| | | ATGTTGCAGTGTCCATCAGTAGAAGTCAGCTTCTTACCTC | |
| | | TCATAGTGAATACAGTTGCTCTGCCTGATGAATTGAGCTA | |
| | | CATATGTACACATGGGGAAGACTGGGATGTAGCTTACATT | |
| | | ATTCATCTTTATCCTTCTCTCACTTTGCGGAATCTTCTCC | |
| | | CATATTCCCTAAGATATTTACTTGAGGGAACAGCAGAAAC | |
| | | TCATGAGCTGGCAGAAGGCAGTACTGCTGATGTTCTGCAT | |
| | | TCGAGAATCAGTGGTGAAATAATGGAATTAGTCCTGGTGA | |
| | | AATACCAGGGCAAAAACTGGAATGGACATTTCCGCATACG | |
| | | TGATACACTACCAGAATTCTTTCCTGTGTGTTTTTCTTCT | |
| | | GACTCCACAGAAGTGACGACAGTCGACCTGTCAGTCCACG | |
| | | TCAGGAGAATTGGCAGCCGGATGGTGCTGTCTGTCTTTAG | |
| | | TCCCTATTGGTTAATCAACAAGACTACCCGGGTTCTCCAG | |
| | | TATCGTTCAGAAGATATTCATGTGAAACATCCAGCTGATT | |
| | | TCAGGGATATTATTTTATTTTCTTTCAAGAAGAAGAACAT | |
| | | TTTTACTAAAAATAAGGTACAATTAAAAATTTCAACCAGT | |
| | | GCCTGGTCCAGTAGTTTCTCATTGGATACAGTGGGAAGTT | |
| | | ATGGGTGTGTGAAGTGTCCTGCCAACAATATGGAGTACCT | |
| | | GGTTGGTGTTAGCATCAAAATGAGCAGTTTCAACCTTTCA | |
| | | CGAATAGTTACCCTGACTCCCTTTTGTACCATTGCAAACA | |
| | | AGTCATCATTAGAACTAGAAGTTGGCGAGATTGCATCTGA | |
| | | TGGCTCAATGCCAACTAATAAATGGAACTATATTGCTTCT | |
| | | TCAGAGTGCCTTCCATTTTGGCCAGAAAGTTTGTCAGGCA | |
| | | AACTTTGTGTGAGAGTGGTGGGCTGTGAAGGATCTTCCAA | |
| | | ACCATTCTTTTATAACCGACAGGATAATGGCACTTTATTG | |
| | | AGCTTAGAAGATCTGAATGGGGGTATCTTGGTGGATGTAA | |
| | | ACACTGCCGAACATTCAACTGTCATAACTTTTTCTGATTA | |
| | | CCATGAGGGATCTGCACCTGCCTTGATAATGAACCATACA | |
| | | CCATGGGACATCCTCACATACAAACAGAGTGGGTCACCAG | |
| | | AAGAAATGGTCTTGCTGCCAAGACAGGCTCGACTTTTTGC | |
| | | CTGGGCAGATCCTACTGGTACCAGAAAACTTACATGGACA | |
| | | TATGCAGCAAATGTTGGGGAACATGATCTGTTAAAGGATG | |
| | | GATGTGGACAGTTTCCATATGATGCAAACATCCAGATACA | |
| | | CTGGGTATCATTTCTGGATGGGCGCCAGAGAGTTTTGCTT | |
| | | TTCACCGATGATGTTGCCTTGGTTTCCAAAGCACTGCAGG | |
| | | CAGAAGAAATGGAACAGGCTGATTATGAAATAACCTTGTC | |
| | | TCTCCACAGTCTTGGGCTTTCACTGGTTAACAATGAAAGC | |
| | | AAGCAGGAAGTTTCCTATATTGGGATAACCAGTTCTGGTG | |
| | | TTGTTTGGGAGGTGAAACCAAAGCAGAAATGGAAGCCATT | |
| | | TAGTCAAAAGCAGATAATCTTATTGGAACAATCCTATCAG | |
| | | AAACATCAAATATCAAGAGACCATGGCTGGATTAAGCTAG | |
| | | ATAATAATTTTGAGGTCAATTTTGATAAAGATCCAATGGA | |
| | | AATGCGCCTCCCTATTCGTAGCCCTATTAAACGAGACTTT | |
| | | TTATCAGGAATTCAGATTGAATTTAAGCAGTCTTCTCACC | |
| | | AGAGAAGTTTAAGGGCCAGGTTGTACTGGCTTCAGGTTGA | |
| | | TAATCAGTTACCAGGTGCAATGTTCCCTGTTGTATTTCAT | |
| | | CCTGTTGCCCCTCCAAAATCTATTGCTTTAGATTCAGAGC | |
| | | CCAAGCCTTTCATTGATGTGAGTGTCATCACAAGATTTAA | |
| | | TGAGTACAGTAAAGTCTTACAGTTCAAGTATTTTATGGTC | |
| | | CTCATTCAGGAAATGGCCTTAAAAATTGATCAAGGGTTTC | |
| | | TAGGAGCTATTATTGCACTGTTTACCCCAACAACAGACCC | |
| | | TGAAGCTGAAAGAAGACGGACAAAGTTAATCCAACAAGAT | |
| | | ATTGATGCTCTAAATGCAGAATTAATGGAGACTTCAATGA | |
| | | CTGATATGTCAATTCTTAGTTTCTTTGAACATTTCCATAT | |
| | | TTCTCCTGTGAAGTTGCATTTGAGTTTGTCTTTGGGTTCC | |
| | | GGAGGTGAAGAATCAGACAAAGAAAAACAGGAAATGTTTG | |
| | | CAGTTCATTCTGTCAACTTGCTGTTGAAAAGCATAGGTGC | |
| | | TACTCTGACTGATGTGGATGACCTTATATTCAAACTTGCT | |
| | | TATTATGAAATTCGATATCAGTTCTACAAGAGAGATCAGC | |
| | | TTATATGGAGTGTTGTTAGGCATTACAGTGAACAGTTCTT | |
| | | GAAACAGATGTATGTCCTTGTATTGGGGTTAGATGTACTT | |
| | | GGAAACCCATTTGGATTAATTAGAGGTCTGTCTGAAGGAG | |
| | | TTGAAGCTTTATTCTATGAACCCTTCCAGGGTGCTGTTCA | |
| | | AGGCCCTGAAGAATTTGCAGAGGGGTTAGTGATTGGAGTG | |
| | | AGAAGCCTCTTTGGACACACAGTAGGTGGTGCAGCAGGAG | |
| | | TTGTATCTCGAATCACCGGTTCTGTTGGGAAAGGTTTGGC | |
| | | AGCAATTACAATGGACAAGGAATATCAGCAAAAAAGAAGA | |
| | | GAAGAGTTGAGTCGACAGCCCAGAGATTTTGGAGACAGCC | |
| | | TGGCCAGAGGAGGAAAGGGCTTTCTGCGAGGAGTTGTTGG | |
| | | TGGAGTGACTGGAATAATAACAAAACCTGTGGAAGGTGCC | |
| | | AAAAAGGAAGGAGCTGCTGGATTCTTTAAAGGAATTGGAA | |
| | | AAGGGCTTGTGGGTGCTGTGGCCCGTCCAACTGGTGGAAT | |
| | | CGTAGATATGGCCAGTAGTACCTTCCAAGGCATTCAGAGG | |
| | | GCAGCAGAATCAACTGAGGAAGTATCTAGCCTCCGTCCCC | |
| | | CTCGCCTGATCCATGAAGATGGCATCATTCGTCCTTATGA | |
| | | CAGACAGGAATCTGAGGGCTCTGACTTACTTGAGCAAGAA | |
| | | CTGGAAATACAGGAATAAATGTTTCCTAAACTACTACTTG | |
| | | ATTTCATCCTTAAAAATCAAAACAAACTGTGGTGTTAATT | |
| | | GACTGTGTGTGAATTCCATTGTCAATTTTAATGAAATTTT | |
| | | CTTTAAAACTCTCACCTCCATCTGAACTTTTCATAGTAGT | |
| | | GGGATTGACTACAAATAAAAACTTGTGGTATTCCTGGTAA | |
| | | TACTGTCCAGAAATAAGAGATTAGTATAAAATATTAAAGG | |
| | | ATGCAGAGAATCAGCTCTCTTCTGCGTTTAATAGATGAAA | |
| | | GCCTTTATTGAGCTCAGAAGCAGATACTGTTACTATCATT | |
| | | TCGAAAATTTTATCTTATGGTGTTCATGTGCATTTCAGGT | |
| | | AAAATTGAAAAACAGGACAATTATTATGTCCAATTAATAT | |
| | | GTTTATGTTTGTGAGTCTTGATGATGGAATTACATAGCTT | |
| | | TCTGTTTCACAAATGGCTCTAAATTTGCTTAAGTTACGGG | |
| | | ACTATTACCTGGAGCATCTGCTTTAATAATTGAATTGTCA | |
| | | GTTGCTCTGAGCCTGCCCTTAGACCTCAAGTAATAAATAG | |
| | | TTGGCACATGAATTTTGAGGATATGTTTCCTCTTCCCTCT | |
| | | TTTTCCTATTTAACCCCTTGGTACTGTTGCTAAATAAATG | |
| | | ATAGCCATTTTATAATTATGTTATATACATTTTCAGCCTT | |
| | | TAGCATTTCTGCTTTTCAAAAATTGAATCTCCTTGTTGGT | |
| | | TATGCTTATTTCATAATTATTAGTTTTAATTAATGTAGAT | |
| | | AGAAGTTGAACATGTAATTAGGCAAATTGCTGTGTGGCAC | |
| | | TTGAATACATAGATTTCTTTATTTTCAAAAACCAACCTTT | |
| | | TGCTTTTAAATCCTTAGAGAGGGTTTATTATCTTAGAGAA | |
| | | AAAATAATTATAATCATTATTTTTGAAATTAGTATCCTCT | |
| | | TAATTCTCAACATAAGTTATGTTTCAATTTCTTTTTTTTG | |
| | | TAATAAATGATGGAAATGTTTAACAATGTCTTATCTAGCA | |
| | | ACTTTCATGCTTCTCCTCAGAAATGAAGCCAAAGTATAAA | |
| | | CTTAGATTTAATGTGTTGTATATTTGAAGAGAATGAAACT | |
| | | ATTAACATATAATTGTTCAGTTGGATTATGTATTTTAAGG | |
| | | ATTGCAGTTATCAAAATAATAAATTGAATGTTTTATGTTT | |
| | | AACCACTTTAAAGAAGAAAGACTGACATCCAAAAACCAGC | |
| | | GTGTGCTAGATATACAAAGGAAATTACTTCTGTCCTTAAG | |
| | | GGACCAAGTATAACAAAACATGTAACTGTTAAAAGTAGCT | |
| | | GACAAACCTTTCTTGTGCCTAGATAATTTAGCATTGGCAA | |
| | | AAATGTCACCACATGCAGTTTTCTAGGAGAGTCAAGCACA | |
| | | AATAACTAATTCAAGATGCTGACTTAAATCATCTCCAATA | |
| | | GTTACCCTTCCTGAGATTCTAAAGTAACAATTTTTAATTT | |
| | | TACTGGTTATATTGCTGTTTTACTGAGACTTACTTTTAAG | |
| | | AACCCCTGTAACTTAAGATTTTTTCTTAATTGTTTTGTTT | |
| | | AGCTCTGTTATTAATTTTTTCCTTGTGATATCTTTTTATA | |
| | | ACTCTCTGTCAAAAAGCACAAAACTTCAAGAAACTTTTAA | |
| | | TTATTTTGTCTGAACATATAATCTTGTCTGATTTCTTAGT | |
| | | TTTTATTAAGATATCAGACAACTTTTAAAACTTTAGTGCA | |
| | | TTATTATAATTACTGGAAGAAAAAGAATGATTATACACTA | |
| | | ATGAGAGGACTTGGTAGTTTTTGTCGTGGATGTCAAGTGT | |
| | | GGGCATGGATAATTGAAATATTTAGGCTATTTCATTCTTT | |
| | | GCCCATCTTGCTGTGATCAGTTAGTTGGGTAAAAATATTT | |
| | | ATTGATTATTTAGACTGTACTGGATATACAAAAGAAGCCT | |
| | | TCTGTCCTTAAGGGACCGAGTAAAACAAAACATGGAAATA | |
| | | TTAAAGAGTATTAGAGTATAAAAGTATATCTTTTTAGCCC | |
| | | TTTGTAATATGGCCAAATTCTAAATAATTTATTTGGGGAT | |
| | | CTTTTGATCCTCATGTTCCTTTTTCTCCTAAGTACTACTT | |
| | | TGTATTCTTTAATATGCAGCTTTGAGAGTTACTGAATCAT | |
| | | ATATTATATTTCCATGAGATGTACTATTCTACTTATCCTC | |
| | | TAATCTTCATATATATATACACACACACATATATATACAC | |
| | | ATACATATATACACACGTACATATATGTACACATACAGAT | |
| | | ATACATACACACAAACACATATATACACACATACATATAC | |
| | | ACACATATATATACACATACAAATATACACATATATACAC | |
| | | ATACATATATATACACACATACAAATATACCCATATGTAC | |
| | | ACATACATATATACACATACATATATACACACACATATAC | |
| | | ACACATATATACGCAAACATACACATATTTACACATACAT | |
| | | ATATACATACATTATATGTATGTATATATAGTCATTTAAT | |
| | | ACTCATTTTGGTTCACATACTTATGATCATGCAACGTTTA | |
| | | AAACAGCATTTCTTGCTTTTTAGTTTTAGTTATATTTTTC | |
| | | CATGTTCTTAGAAATGCCTCATTAACATTTTTAATTCTTG | |
| | | TATTGCCATCTATTGAGGTGACATTACATTGTGTTTTTAT | |
| | | CTCGTCTTAATTCATGACATTAAATTATTCTACTAACAGT | |
| | | AATAATGCTGTAATAAACATCATTATAGATTTTGCTTTTT | |
| | | TATATCTTGTTTGCTTTTTCATATTTCCTTAGAATTTACT | |
| | | TGAAAAAATTGAATTACTGGGTAAAGGGCTTTTGCAAAGT | |
| | | ATTGTTAAATTCCTCGAGTTGCATTTTTGGAAAGGGGACG | |
| | | TGAATATTTTATCAACTAATTTGGTCTCCCTGCTGCCATT | |
| | | AGTGACTGAATATCTTAATCTGAATCTCAGAGTGTAGTGG | |
| | | GTTTTTAGTAGTGCTGAAGACAAGTTTTCTAAAGTGTATT | |
| | | ATGGTGATAAATTATATTTTAAAAACTGTCAATGGCTTGA | |
| | | AGCACAATAGCCTAATAACTAACGAAAATACATACAAGAT | |
| | | AGAAAGTGGGTAGTATTTCTTGTACTTGCATTTCAGATCT | |
| | | AAATATTTTAACATATTTAAATTTCAAGCTGCAGATAAAT | |
| | | GCATTACATTATTAAATTCATTTCCCATTTTCTCTTTGAA | |
| | | GAAATTAAGGCAAAAGTGTTAAAAGATTTTAACTAATTCG | |
| | | CACAAGTGAATTGTGAAACAAGTAGCTATTGCTGTGAAAT | |
| | | CTGCACTCCTCTCTGAGACTCATTCTGAAGATGAGATCCC | |
| | | AGTTCTTTGTGGATTCCTCTTCCTTATTCATGGCTTTTTG | |
| | | CAATTGTCAAGGAATGACTAGGTACCAAGCAACTTTAAAA | |
| | | AATGTATATTTAAGCATTGAAATAATATCAAATGTGATTT | |
| | | CTCTGCTTGTGGTTATATTGATTATATTATCCTTTTAATA | |
| | | ATATTGGCATTATATTCTTGGTCGTAAAATGTCAAGGTCT | |
| | | TATTTATTCAGTATATTTATGTTCTGTATTTTCATATATA | |
| | | TTATCTATTTTCAGCCATGCATTATATATAATGTCAGTAA | |
| | | TAGTATTTCATTAGCATTCATTATAAAAAAACTCGTTTTT | |
| | | AATATTTGACTAATTCAAGTCACAGTACTTTTGAGATAGC | |
| | | TGAAAAGGAAAATAAATGTGTTTTAATGTGCTACTAAAAA | |
| | | AAAAAAA | |
| WDFY3 | NM_014991.4 | GCGGCCGCAGAATCGAGCTCGGGCCCCGGCCCCCGGCCCG | 49 |
| | | CGGCGCGGGGCTCCCGGGCCCCGCCGCGGACGTCGCGCCG | |
| | | GTCGCCCCTTCCCCGTAGCCCGTGCGCCCTCGGCGCGGAG | |
| | | CCCCGGCCCGCCGCGGTCCCGTCTCCTGGGCCTGTCCCGC | |
| | | CCGCGCCCTCCGCCGGCCCTCAGGTATAATACTTCTCCAC | |
| | | GTCTGCTTCAGGAAGAAAGTGCCTGCCATTCTTATCATTT | |
| | | CTAAGCAGGTTCATGCCAGCCCAGAACAGAGAATCAGCTG | |
| | | GAGCCCAGATTTCAAGTTTTGAGTAAAATACCTTCAAGCG | |
| | | AATGGGCCCTATTGTGCTCACACATTCAGAACCTGTTACC | |
| | | CAAGGAATTCCCTAAAGAATTAGAAGTGCGTCTCACCAAC | |
| | | CAGCCAAGATGAACATGGTGAAGAGGATCATGGGGCGGCC | |
| | | GAGGCAGGAGGAGTGCAGCCCACAAGACAACGCCTTAGGA | |
| | | CTGATGCACCTCCGCCGGCTCTTCACGGAGTTGTGCCATC | |
| | | CTCCCCGGCACATGACTCAGAAGGAACAAGAAGAGAAACT | |
| | | GTATATGATGCTGCCAGTGTTTAACAGGGTTTTTGGAAAT | |
| | | GCTCCGCCGAATACAATGACAGAAAAATTTTCTGATCTTC | |
| | | TGCAGTTCACAACACAAGTCTCACGACTAATGGTGACAGA | |
| | | AATTCGAAGGAGAGCATCAAACAAATCCACAGAGGCTGCA | |
| | | AGTCGGGCCATAGTTCAGTTCCTAGAGATTAATCAGAGTG | |
| | | AAGAAGCCAGTAGAGGCTGGATGCTTCTAACGACAATTAA | |
| | | TTTGTTAGCTTCCTCTGGTCAGAAAACCGTGGACTGCATG | |
| | | ACAACAATGTCAGTGCCTTCCACCCTGGTTAAATGTTTAT | |
| | | AGGTGCACAGAATGAGCTACCTCTAGCAGAACGTCGAGGA | |
| | | CTACTCCAGAAAGTTTTTGTACAGATCTTAGTGAAACTGT | |
| | | GCAGTTTTGTTTCCCCTGCGGAGGAGCTGGCTCAGAAAGA | |
| | | TGATCTCCAGCTTCTATTCAGTGCAATAACCTCTTGGTGC | |
| | | CCTCCCTATAACCTGCCTTGGAGAAAGAGTGCTGGAGAAG | |
| | | TCCTCATGACCATATCTCGTCATGGTCTTAGTGTCAATGT | |
| | | AGTGAAGTATATTCATGAGAAAGAGTGTTTATCTACATGT | |
| | | GTTCAGAATATGCAGCAATCAGATGACCTGTCTCCCCTAG | |
| | | AAATTGTCGAAATGTTTGCTGGGCTTTCTTGTTTCCTCAA | |
| | | AGATTCCAGCGATGTTTCCCAAACACTTCTGGATGATTTT | |
| | | CGGATATGGCAAGGATATAATTTTCTTTGTGATCTCTTGC | |
| | | TTAGATTGGAACAAGCAAAAGAGGCAGAATCCAAAGATGC | |
| | | CTTGAAAGATCTGGTTAATCTGATAACTTCCCTAACAACA | |
| | | TATGGTGTCAGTGAACTAAAACCAGCTGGTATTACCACAG | |
| | | GGGCACCCTTTTTATTGCCTGGATTTGCAGTACCTCAGCC | |
| | | TGCAGGCAAAGGTCACAGTGTGAGAAACGTCCAGGCCTTT | |
| | | GCAGTTCTTCAGAATGCATTTTTAAAAGCAAAAACCAGCT | |
| | | TCCTTGCCCAAATCATCCTTGATGCTATCACAAATATTTA | |
| | | CATGGCTGACAATGCCAATTACTTCATCCTAGAGTCACAG | |
| | | CACACATTGTCACAGTTTGCAGAGAAGATTTCTAAACTCC | |
| | | CAGAAGTACAAAACAAATACTTTGAGATGCTGGAGTTTGT | |
| | | TGTTTTTAGCTTAAATTATATACCTTGTAAAGAACTTATT | |
| | | AGTGTCAGTATCCTCTTAAAATCTAGCTCTTCTTATCACT | |
| | | GTAGCATTATTGCAATGAAAACACTTCTTAAGTTTACAAG | |
| | | ACATGACTACATATTTAAAGACGTGTTCAGGGAGGTTGGC | |
| | | CTTTTGGAGGTCATGGTAAACCTTTTGCATAAATATGCTG | |
| | | CCCTGTTGAAGGATCCAACTCAGGCACTAAATGAACAAGG | |
| | | GGACTCAAGAAATAATAGTTCAGTTGAAGACCAAAAACAC | |
| | | CTGGCTTTATTGGTTATGGAGACCTTGACAGTGCTTCTTC | |
| | | AAGGATCAAACACAAATGCAGGAATTTTTCGAGAATTTGG | |
| | | AGGTGCAAGATGTGCACATAATATAGTAAAGTACCCTCAA | |
| | | TGCCGGCAGCATGCCTTGATGACTATCCAACAGCTGGTGC | |
| | | TCTCCCCAAATGGGGACGATGACATGGGCACTCTCCTGGG | |
| | | GCTAATGCATTCAGCCCCACCGACGGAATTGCAGTTGAAG | |
| | | ACTGATATTTTAAGGGCCCTCCTGTCGGTCCTTCGAGAAA | |
| | | GCCATCGTTCAAGAACAGTTTTTAGGAAAGTTGGAGGATT | |
| | | TGTGTACATTACATCCTTGCTCGTTGCTATGGAAAGATCT | |
| | | TTGAGCTGTCCACCCAAGAATGGCTGGGAGAAAGTGAACC | |
| | | AGAATCAAGTGTTTGAACTTCTTCACACTGTGTTCTGCAC | |
| | | GTTGACTGCAGCAATGCGCTATGAGCCAGCCAACTCTCAT | |
| | | TTCTTCAAAACAGAGATTCAGTATGAGAAGTTGGCAGATG | |
| | | CTGTTCGATTTCTTGGCTGCTTCTCAGACCTAAGAAAAAT | |
| | | AAGCGCCATGAATGTCTTCCCCTCAAATACACAGCCATTT | |
| | | CAAAGACTTTTAGAGGAAGATGTAATCTCAATAGAATCAG | |
| | | TGTCACCCACGTTACGGCACTGCAGTAAACTTTTTATTTA | |
| | | TCTTTACAAAGTAGCCACAGATTCTTTTGACAGTCGTGCA | |
| | | GAACAGATCCCTCCTTGCCTGACAAGTGAGTCTTCTCTCC | |
| | | CCTCTCCTTGGGGTACACCAGCTTTGTCCAGGAAAAGGCA | |
| | | TGCATATCATTCTGTTTCAACTCCCCCTGTTTACCCTCCT | |
| | | AAAAATGTTGCCGACCTGAAACTACATGTGACAACTTCAT | |
| | | CTCTGCAGAGTTCTGATGCAGTCATCATTCATCCTGGAGC | |
| | | CATGCTTGCCATGCTGGACCTACTGGCCTCTGTTGGGTCA | |
| | | GTGACACAGCCAGAACATGCTTTGGATCTTCAACTTGCCG | |
| | | TGGCAAATATTTTACAATCCCTGGTGCACACAGAAAGGAA | |
| | | CCAGCAAGTCATGTGTGAAGCTGGTCTTCATGCACGACTG | |
| | | CTGCAGAGGTGCAGTGCTGCATTGGCTGATGAGGACCACT | |
| | | CACTGCACCCGCCCCTGCAGCGGATGTTTGAACGATTAGC | |
| | | CTCTCAGGCTCTGGAACCCATGGTGTTGAGGGAGTTTTTA | |
| | | CGTTTGGCAAGTCCTTTAAATTGTGGTGCCTGGGACAAAA | |
| | | AACTGCTAAAACAATATAGGGTCCACAAACCAAGTTCACT | |
| | | GAGTTATGAACCAGAAATGAGAAGTAGTATGATCACATCT | |
| | | CTGGAAGGTCTGGGTACTGATAATGTTTTTAGCTTACATG | |
| | | AAGATAACCATTACCGGATAAGCAAGAGCCTGGTAAAATC | |
| | | TGCGGAAGGAAGTACTGTACCCCTGACCAGGGTGAAGTGT | |
| | | CTGGTCTCCATGACAACCCCACATGACATCAGACTTCATG | |
| | | GGTCATCAGTTACTCCAGCTTTTGTTGAATTTGACACATC | |
| | | ACTTGAAGGGTTTGGATGTCTTTTTTTGCCCAGTTTGGCC | |
| | | CCTCATAATGCTCCTACAAATAATACCGTCACAACAGGTC | |
| | | TTATTGATGGGGCTGTGGTCAGTGGCATTGGTTCTGGTGA | |
| | | AAGATTCTTCCCTCCTCCCTCCGGCTTAAGTTACTCTAGC | |
| | | TGGTTTTGTATTGAACATTTTAGTTCTCCTCCAAATAACC | |
| | | ACCCTGTCAGACTTCTTACTGTTGTGCGCCGAGCAAATTC | |
| | | TTCTGAGCAACATTACGTGTGCCTTGCAATAGTTCTATCA | |
| | | GCAAAAGACCGATCTCTGATTGTTTCCACCAAAGAGGAAC | |
| | | TCCTCCAAAATTATGTTGATGATTTTAGTGAAGAGTCCTC | |
| | | ATTTTATGAAATTCTCCCATGCTGTGCTCGCTTTCGATGT | |
| | | GGAGAGCTTATCATTGAGGGACAGTGGCATCATTTGGTCC | |
| | | TGGTAATGAGCAAAGGCATGTTGAAAAACAGTACTGCAGC | |
| | | CCTTTATATTGATGGACAGCTTGTTAACACTGTAAAGCTT | |
| | | CATTATGTCCACAGTACTCCAGGGGGTTCAGGTTCGGCAA | |
| | | ATCCACCAGTGGTGAGCACGGTCTATGCCTACATTGGTAC | |
| | | TCCACCTGCCCAACGCCAAATTGCCTCATTGGTTTGGCGC | |
| | | CTGGGACCCACACATTTTCTAGAAGAAGTTTTACCTTCTT | |
| | | CAAATGTTACTACCATTTATGAACTTGGACCAAATTATGT | |
| | | TGGAAGCTTTCAGGCTGTATGTATGCCATGTAAAGATGCA | |
| | | AAATCCGAAGGGGTGGTGCCATCCCCTGTGTCATTAGTAC | |
| | | CAGAGGAGAAAGTGTCATTTGGCCTCTATGCACTCTCTGT | |
| | | GTCGTCTCTAACAGTGGCAAGAATCCGGAAAGTGTATAAC | |
| | | AAATTGGATAGCAAAGCCATTGCTAAGCAGTTAGGCATTT | |
| | | CCTCACATGAGAATGCCACTCCTGTGAAGTTGATACACAA | |
| | | TTCAGCAGGACATCTTAATGGATCTGCACGGACAATTGGG | |
| | | GCCGCTCTGATTGGATACTTGGGAGTAAGAACATTTGTCC | |
| | | CTAAGCCTGTTGCCACTACTTTGCAGTACGTTGGTGGAGC | |
| | | TGCAGCCATCCTGGGCCTGGTGGCCATGGCCTCTGATGTG | |
| | | GAAGGGTTATATGCAGCAGTCAAGGCCCTGGTTTGTGTGG | |
| | | TCAAGAGTAACCCACTAGCCAGCAAAGAAATGGAAAGAAT | |
| | | CAAGGGCTACCAGTTGCTGGCAATGTTGCTTAAGAAGAAA | |
| | | CGTTCCCTTCTTAACAGCCACATCCTCCATCTAACTTTTT | |
| | | CTTTGGTGGGAACTGTTGATAGTGGACATGAGACCTCCAT | |
| | | TATTCCAAATTCAACTGCTTTCCAGGACCTCCTCTGTGAT | |
| | | TTTGAAGTCTGGCTCCATGCACCATATGAACTTCATCTTT | |
| | | CCTTATTTGAACACTTTATTGAACTGCTCACAGAGTCCAG | |
| | | TGAAGCCTCAAAGAATGCCAAATTAATGAGAGAATTCCAG | |
| | | TTAATCCCAAAGCTGCTCCTGACTCTTCGAGATATGTCTT | |
| | | TATCCCAGCCTACTATTGCTGCTATTAGTAATGTCCTGAG | |
| | | CTTCTTACTGCAAGGTTTTCCTAGCAGCAATGATCTGCTC | |
| | | AGATTTGGGCAGTTTATTTCTTCTACTTTGCCAACCTTTG | |
| | | CGGTTTGTGAGAAATTTGTAGTAATGGAAATAAATAATGA | |
| | | AGAGAAGCTTGACACTGGAACTGAAGAGGAGTTTGGAGGT | |
| | | CTTGTATCAGCTAATCTTATACTTTTGAGGAACAGACTTC | |
| | | TGGATATCTTGCTAAAACTAATTTATACATCTAAAGAAAA | |
| | | GACAAGCATTAATTTGCAAGCTTGTGAAGAACTGGTGAAG | |
| | | ACACTGGGTTTTGACTGGATCATGATGTTTATGGAGGAAC | |
| | | ACTTACATTCCACCACAGTTACAGCAGCCATGAGGATTCT | |
| | | TGTTGTCCTACTAAGTAATCAGTCTATTCTCATCAAGTTT | |
| | | AAAGAAGGACTCAGTGGTGGAGGATGGCTTGAACAGACAG | |
| | | ATTCTGTCTTAACTAATAAGATTGGAACTGTATTAGGATT | |
| | | CAACGTGGGCAGAAGTGCTGGTGGGAGATCGACGGTCAGG | |
| | | GAGATTAACCGAGATGCTTGTCATTTTCCTGGTTTTCCAG | |
| | | TCCTTCAGTCATTCCTTCCTAAACACACTAATGTCCCTGC | |
| | | CCTCTATTTTCTCCTCATGGCCTTGTTTCTGCAGCAGCCA | |
| | | GTTAGTGAGCTGCCTGAGAACCTGCAGGTCAGTGTGCCTG | |
| | | TCATCAGCTGCCGGAGTAAGCAGGGTTGCCAGTTTGATTT | |
| | | GGATTCCATTTGGACATTCATCTTTGGAGTTCCTGCCTCC | |
| | | AGCGGAACTGTGGTCTCTTCTATCCATAACGTATGCACAG | |
| | | AAGCTGTTTTTTTATTATTGGGAATGCTCCGCAGCATGCT | |
| | | GACTTCACCTTGGCAATCAGAAGAAGAGGGATCTTGGCTC | |
| | | CGAGAATATCCTGTGACCCTGATGCAGTTCTTCAGATATT | |
| | | TGTATCACAACGTGCCAGACCTTGCCTCCATGTGGATGAG | |
| | | CCCTGACTTCCTGTGTGCATTAGCAGCCACCGTCTTCCCC | |
| | | TTCAATATTCGCCCTTACTCAGAGATGGTGACTGACCTTG | |
| | | ATGATGAAGTTGGATCTCCAGCAGAAGAGTTTAAAGCGTT | |
| | | TGCAGCAGACACAGGGATGAACAGGAGCCAATCAGAGTAC | |
| | | TGCAATGTGGGCACCAAGACATATCTGACCAATCACCCGG | |
| | | CTAAAAAGTTCGTTTTTGACTTCATGCGGGTCTTAATCAT | |
| | | AGACAACCTCTGTCTCACTCCTGCCAGCAAGCAAACTCCA | |
| | | CTAATTGATCTTTTGTTGGAGGCTTCCCCTGAAAGGTCTA | |
| | | CAAGAACTCAGCAAAAAGAATTTCAAACTTACATTTTGGA | |
| | | TAGCGTGATGGACCATTTGCTTGCAGCTGATGTGTTATTA | |
| | | GGGGAAGATGCATCTCTGCCTATTACCAGTGGAGGAAGCT | |
| | | ACCAGGTATTGGTGAACAATGTGTTTTATTTCACACAGCG | |
| | | TGTGGTGGACAAGCTTTGGCAAGGCATGTTCAACAAAGAA | |
| | | TCTAAACTTCTTATAGATTTTATAATTCAACTAATTGCAC | |
| | | AGTCAAAGAGAAGATCACAGGGATTGTCACTGGATGCAGT | |
| | | GTATCATTGCCTCAATAGGACCATCTTGTACCAGTTCTCA | |
| | | CGGGCACACAAAACCGTTCCTCAGCAAGTAGCTCTGCTTG | |
| | | ATTCACTCAGGGTCCTCACTGTAAACAGAAACTTGATCCT | |
| | | GGGACCTGGGAACCATGACCAAGAATTCATTAGCTGTCTG | |
| | | GCCCACTGCTTGATAAATCTACATGTTGGAAGCAACGTGG | |
| | | ATGGATTTGGACTGGAAGCAGAAGCCCGCATGACCACATG | |
| | | GCACATTATGATCCCCTCGGACATTGAACCAGATGGTAGT | |
| | | TACAGCCAAGATATTAGTGAAGGGCGTCAGCTTCTCATAA | |
| | | AAGCTGTCAACAGAGTTTGGACTGAACTGATACATAGTAA | |
| | | GAAACAAGTCTTAGAGGAACTTTTCAAAGTAACTCTACCT | |
| | | GTGAATGAAAGGGGCCACGTGGACATAGCTACAGCAAGGC | |
| | | CACTCATTGAAGAAGCTGCCCTGAAGTGCTGGCAGAATCA | |
| | | TTTGGCCCATGAAAAGAAATGCATAAGTCGAGGAGAAGCT | |
| | | TTAGCGCCCACCACACAGTCCAAATTATCCCGTGTCAGCA | |
| | | GTGGCTTTGGTCTTTCCAAGTTAACAGGATCAAGAAGGAA | |
| | | TCGAAAAGAAAGTGGTCTTAATAAACACAGTCTTTCCACC | |
| | | CAGGAGATTTCGCAGTGGATGTTTACTCACATTGCTGTTG | |
| | | TTCGTGACTTAGTAGATACACAATATAAAGAATATCAGGA | |
| | | GCGTCAGCAGAATGCCCTGAAGTACGTGACAGAAGAGTGG | |
| | | TGTCAGATCGAGTGCGAGCTGTTGAGGGAGCGGGGGCTGT | |
| | | GGGGCCCTCCCATCGGCTCCCACCTCGACAAGTGGATGCT | |
| | | GGAGATGACAGAAGGGCCCTGCAGGATGAGGAAAAAGATG | |
| | | GTGCGAAATGATATGTTTTATAACCATTACCCTTACGTGC | |
| | | CAGAAACTGAGCAAGAGACAAATGTGGCGTCTGAGATCCC | |
| | | AAGTAAACAGCCTGAGACACCCGATGATATTCCTCAAAAG | |
| | | AAACCTGCTCGATATAGAAGAGCCGTAAGTTATGACAGTA | |
| | | AAGAGTACTACATGCGACTGGCCTCTGGCAATCCCGCCAT | |
| | | TGTCCAAGACGCCATTGTGGAGAGTTCAGAAGGTGAAGCT | |
| | | GCTCAGCAAGAACCAGAGCATGGGGAAGACACTATTGCTA | |
| | | AAGTCAAAGGTTTGGTCAAGCCTCCTCTAAAACGCTCCCG | |
| | | ATCTGCACCTGATGGAGGAGATGAGGAGAACCAGGAGCAG | |
| | | CTACAAGACCAGATTGCTGAGGGCAGCTCCATAGAAGAGG | |
| | | AGGAGAAAACAGATAATGCTACCTTACTGCGCCTGTTAGA | |
| | | GGAAGGAGAAAAGATCCAACACATGTACCGCTGTGCTCGA | |
| | | GTCCAGGGCCTAGATACCAGTGAGGGGCTCCTTCTTTTTG | |
| | | GTAAAGAGCATTTTTATGTGATTGATGGATTTACCATGAC | |
| | | AGCAACCAGGGAAATAAGAGATATTGAAACCTTACCTCCA | |
| | | AATATGCATGAGCCTATTATTCCTAGAGGAGCCAGGCAAG | |
| | | GCCCTAGTCAACTCAAGAGAACATGCAGCATTTTTGCATA | |
| | | TGAAGATATCAAGGAAGTTCATAAAAGGAGATATCTCCTG | |
| | | CAGCCTATTGCTGTGGAAGTTTTCTCTGGAGATGGACGGA | |
| | | ATTACCTCCTTGCTTTTCAGAAAGGAATCAGAAACAAAGT | |
| | | CTATCAAAGGTTTTTGGCTGTAGTGCCATCTCTAACGGAC | |
| | | AGTTCAGAATCTGTATCTGGGCAACGACCAAACACGAGTG | |
| | | TGGAGCAGGGATCTGGGTTACTTAGCACTTTGGTTGGAGA | |
| | | GAAGTCTGTGACTCAGAGATGGGAGAGAGGTGAAATCAGC | |
| | | AACTTCCAATATTTGATGCATTTGAACACTTTGGCTGGCA | |
| | | GATCATATAATGATCTCATGCAGTATCCTGTCTTCCCCTG | |
| | | GATCCTTGCAGATTATGACTCAGAGGAGGTGGATCTTACT | |
| | | AATCCCAAGACGTTTAGAAACCTGGCTAAGCCAATGGGAG | |
| | | CACAAACAGATGAACGATTAGCTCAGTATAAGAAGCGGTA | |
| | | TAAAGACTGGGAGGATCCTAATGGAGAAACTCCTGCATAC | |
| | | CACTATGGGACCCACTATTCATCTGCAATGATTGTGGCCT | |
| | | CATACCTTGTAAGGATGGAGCCTTTCACACAGATATTCTT | |
| | | AAGGCTACAGGGTGGCCACTTTGACCTGGCTGACCGGATG | |
| | | TTTCACAGTGTGCGCGAGGCCTGGTATTCAGCGTCAAAGC | |
| | | ACAATATGGCAGATGTAAAAGAACTTATCCCAGAGTTCTT | |
| | | TTATTTACCAGAATTCCTGTTCAATTCCAACAACTTTGAT | |
| | | CTAGGCTGTAAACAAAATGGCACCAAGCTTGGAGATGTTA | |
| | | TCCTTCCACCCTGGGCAAAAGGGGACCCACGAGAATTCAT | |
| | | CAGAGTCCATCGTGAGGCTTTGGAGTGTGATTACGTGAGT | |
| | | GCCCATCTACATGAGTGGATTGACTTAATCTTCGGTTATA | |
| | | AACAGCAAGGCCCTGCTGCAGTAGAAGCTGTAAATGTCTT | |
| | | CCATCATCTTTTTTATGAGGGTCAAGTGGATATCTACAAC | |
| | | ATCAATGACCCACTAAAGGAGACAGCCACAATTGGGTTCA | |
| | | TTAATAACTTCGGTCAGATCCCTAAACAGTTATTTAAAAA | |
| | | ACCTCATCCACCAAAGCGAGTGAGAAGTCGACTCAATGGA | |
| | | GACAATGCAGGAATCTCTGTCCTACCAGGATCTACAAGTG | |
| | | ACAAGATCTTTTTTCATCATCTAGACAACTTGAGGCCTTC | |
| | | TCTAACACCTGTAAAAGAACTCAAAGAACCTGTAGGACAA | |
| | | ATCGTATGTACAGATAAAGGTATTCTTGCGGTGGAACAGA | |
| | | ATAAGGTTCTTATCCCACCAACCTGGAATAAAACTTTTGC | |
| | | TTGGGGCTATGCAGACCTCAGTTGCAGACTGGGAACCTAT | |
| | | GAGTCAGACAAGGCCATGACTGTTTATGAATGCTTGTCTG | |
| | | AGTGGGGCCAGATTCTCTGTGCAATCTGCCCCAACCCCAA | |
| | | GCTGGTCATCACGGGTGGAACAAGCACGGTTGTGTGTGTG | |
| | | TGGGAGATGGGCACCTCCAAAGAAAAGGCCAAGACCGTCA | |
| | | CCCTCAAACAGGCCTTACTGGGCCACACTGATACCGTCAC | |
| | | CTGCGCCACAGCATCATTAGCCTATCACATAATTGTCAGT | |
| | | GGGTCCCGTGATCGAACCTGTATCATTTGGGATTTGAACA | |
| | | AACTGTCATTTCTAACCCAGCTTCGAGGGCATCGAGCTCC | |
| | | AGTTTCTGCTCTTTGTATCAATGAATTAACAGGGGACATT | |
| | | GTGTCCTGCGCTGGCACATATATCCATGTGTGGAGCATCA | |
| | | ATGGGAACCCTATCGTGAGTGTCAACACGTTCACAGGTAG | |
| | | GAGCCAGCAGATCATCTGCTGCTGCATGTCGGAGATGAAC | |
| | | GAATGGGACACGCAGAACGTCATAGTGACAGGACACTCAG | |
| | | ATGGAGTGGTTCGGTTTTGGAGAATGGAATTTTTGCAAGT | |
| | | TCCTGAAACACCAGCTCCTGAGCCTGCTGAAGTCCTAGAA | |
| | | ATGCAGGAAGACTGTCCAGAAGCACAAATAGGGCAGGAAG | |
| | | CCCAAGACGAGGACAGCAGTGATTCAGAAGCAGATGAGCA | |
| | | GAGCATCAGCCAGGACCCTAAGGACACTCCAAGCCAACCC | |
| | | AGCAGCACCAGCCACAGGCCCCGGGCAGCCTCCTGCCGCG | |
| | | CAACAGCCGCCTGGTGTACTGACAGTGGCTCTGACGACTC | |
| | | CAGACGCTGGTCCGACCAGCTCAGTCTAGATGAGAAAGAC | |
| | | GGCTTCATATTTGTGAACTATTCAGAGGGCCAGACCAGAG | |
| | | CCCATCTGCAGGGCCCCCTTAGCCACCCCCACCCCAATCC | |
| | | CATTGAGGTGCGGAATTACAGCAGATTGAAACCTGGGTAC | |
| | | CGATGGGAACGGCAGCTGGTGTTCAGGAGTAAGCTGACTA | |
| | | TGCACACAGCCTTTGATCGAAAGGACAATGCACACCCAGC | |
| | | TGAGGTCACTGCCTTGGGCATCTCCAAGGATCACAGTAGG | |
| | | ATCCTCGTTGGTGACAGTCGAGGCCGAGTTTTCAGCTGGT | |
| | | CTGTGAGTGACCAGCCAGGCCGTTCTGCTGCTGATCACTG | |
| | | GGTGAAGGATGAAGGTGGTGACAGCTGCTCAGGCTGCTCG | |
| | | GTGAGGTTTTCACTCACAGAAAGACGACACCATTGCAGGA | |
| | | ACTGTGGTCAGCTCTTCTGCCAGAAGTGCAGTCGCTTTCA | |
| | | ATCTGAAATCAAACGCTTGAAAATCTCATCCCCGGTGCGT | |
| | | GTTTGTCAGAACTGTTATTATAACTTACAGCATGAGAGAG | |
| | | GTTCAGAAGATGGGCCTCGAAATTGTTGAAGATTCAACAA | |
| | | GCTGAGTGGAGACCATGGTCTGTAGACCCCTTCCCGATTC | |
| | | TCCTGTCCCAGCTTGGAAGGCATTGAAAACAGTCTCCGTT | |
| | | TACACATCTCTTCATACCACGTGTTTGAAGTGTTAAAATT | |
| | | CAAAGGGATCATTGAATAAAACGGGTGTAGAGTACAGGAA | |
| | | TGGGGCAGACGCGATTCAGGTGAACAGCACAAGAAGAATA | |
| | | TGAGGTGGTTCCTAGGAGCAACACTTTCGACCTCCAGTTC | |
| | | TCCCTGATGACAGTAGCTGTCTCCAAGAGAAAAATCCTCA | |
| | | CTTATTAACTCTCTTTTCTTGCATCTCATTTTTATAGAGC | |
| | | TACTCATCCTTATTTGGAAAAACCAACAACAAAAAAGGCT | |
| | | TTTAGAAAATGGTTGTAAATCTGACTTCTTTGCAAGTAAC | |
| | | TATGTATATTGTAAATAGATATAAAAGGCCTTTTTTCTAA | |
| | | ATAAGGACTTAACTGCCTGTAACATGAAACTTCAAACTAA | |
| | | ACCACTAACTCAATGAACTACTTATGGTTTGTCTGACATC | |
| | | CCTCACTTACCAATTAATTATAAATATGTTTTTTTAAATC | |
| | | CCCAAAGACATTATCTGTGGTCTTTTTTTCCTTTCAAGCT | |
| | | CAGCCTGTGTGCCTGATGTCATTTCTTTCAAGTTGCCCAC | |
| | | AGTATCTCCACTTAAACTAGGCTAGTAACCAAAATAATGT | |
| | | GGACCTTCTTTAGGAAACAGTGTGGGAGAATAGGAGTCCA | |
| | | GCCGTAAGATAAACTGGAAATATTTGGGCGTCTTGTACCT | |
| | | GGCTACGCACCACCTCAGTGTTGTTCCTACATAAACAGGG | |
| | | CCCCTTTTAAACTTGTATGTGGACTGCTGTTTGGTCAAAG | |
| | | AATACCTTCTTAGCATTGCAGAAAGGTGGTCAGATGACCA | |
| | | GTGTAGTGCAGGAAACAGCCCTGTCTCAACTAATGGAAAT | |
| | | ATATTTGCATGTAACCCAAAATTAGCTTATCTTGCATAGA | |
| | | ACATAATAAGTATGTGTCTTTGGTGACACTAATGTTCTAC | |
| | | TATAGCTTATTTTCAAACAAGGGGTAAAAAAAGGAAAGAA | |
| | | AGAAGTGTACAGAATTAACATATAAACTTTGTTGTAAAAC | |
| | | TGAATCATGTCAGAACTGCTTAAAATTAACCTTTACCATT | |
| | | TAATGTCATCTACCTGAAAACAGTGAGATTTATACTGTAT | |
| | | CAATGTCTATTTTTTTGTTTTTGCTATGAATATAATTACA | |
| | | GTATTTTAATATTTAGTTATTTAATTTGTTCTACTAGTTG | |
| | | GATACAGAACACACAAATCCAGGGGGATTAAAGCTGGAAG | |
| | | GGGCTAAGAGATTAGTTTACAGAGAAAAGGCTTGGTGGTG | |
| | | GGATTTTTTTAAATGTGTGTTATGTACATATATATATATA | |
| | | TATAATATATATTAAAAATGAAACAATTAATCTAGATTTT | |
| | | AACATTTTCAGAAACTTAGTGATAACATTATGAACAATTC | |
| | | TAAAAGCCCTGTGATTTGAAAAATATAGAATCATTAATGG | |
| | | CCCAAGATAGGCCTTCACACCTTCACAGGTGCGAAAGGAA | |
| | | AGGCCTTCACACCCTCACAGAGGCATCATGCAAAGGACAG | |
| | | CGGCTTTGGCTTTTCCAATTTTCCATCTTTAGGCCCTGGT | |
| | | GAGAGGCACACTTATGCACTAAAATGCACATATATGCACA | |
| | | TGCATTCAAAAATAGGCATTTGGTACAATGGTGATCTTGT | |
| | | ACCTGATGGGCTGAAACCAGCTTAAGAACAAATTTGTTCT | |
| | | TCCTGATATGATAACTAGGTCTCCAAGAGAAAATAGAAAG | |
| | | GCTGCTTTAGTGCCTTACGCTTACTAAATTTAAATCTTTA | |
| | | TTTACCTGGGTTTGAGCCTACAGTCTATTTATGATTACAT | |
| | | ATCAAAATTGATTAAAACACTTCCATTTCTAAAAGTTCAA | |
| | | ATATACTTGTTAATAAAAGGATTATCGGCATTAATACTTT | |
| | | AATTTAAAGAAAAGTTGTGTTCTGTTTTCCTTTCTGTGTC | |
| | | TTACTCCCCCCACACTCTCCCTCCCCCATCACCATCTTCA | |
| | | ATTCTAATAAATAATGCTGATGTTCAACAGTTGCAGAAAT | |
| | | TGTGCTATTATGTAACTGTGGGCCTTGCCCCTGTCTGGCC | |
| | | CTCTAGATGATTTGTAGCAGTGTTATTCTACACTTTTTAA | |
| | | AAGAAGCGTCCTCCTTTTGTCCATGAATCATGTTTACCCC | |
| | | ATACCCAGTGGCAGAGGTGTTCTTTAAAGACTTGAATATA | |
| | | TGAATGTGTGTGTGTAGTTACTTAAAGGTTATTCCTCTTT | |
| | | GTAATAGGAAACTATATGGGATGAACACTTTTAAACTTTC | |
| | | CGACACAACTTCCATTACTAACTTTCTAACAGAACTTCCA | |
| | | TAACTAGAAGGTGGAAACCAAAACCCTCATGGTAGTATTT | |
| | | CCTCTGGCAGCTGGTGCTGTGGGCAACTGTTTTGTTCAAT | |
| | | CGGGTTTCTTTTCTTTTTGCCTCTAATGCAGAAATCAACA | |
| | | GAATCACTCACACATACAAGTACACTCACATACATAAACT | |
| | | AATTATTTCTCTGGATATCTTTCTGTGTTCCATGTAAATT | |
| | | TATTTACCAACATCTATTGTCAACATGTACATCTACCTTA | |
| | | GTATGGTCTGCATTCTTTTTCTGAGAGTACCTCATAGGGC | |
| | | TCCTGCCTGATCTTTGTAGTTTGTTCATTCATCCATCCAC | |
| | | CTGTTCATTTGTTCATCCATGTATTCTAACATTTCTATGT | |
| | | AGTGTGCAACTCTAATGTCATGCTTTTGAAGAAGAGAATA | |
| | | GCTGCCCATAGCAGCCATCCGTCTGGATAATAGCAAAACA | |
| | | CTCTAGATAAGTTATTTTGCACTTTCTTATGTATAAAGTT | |
| | | GGTAGAAACTTATTTTTGCTTTGTATCATTTAAATACATT | |
| | | TTGTTTTGGTAAATGAACTGTGTATAAAATATTTATGCCG | |
| | | TTAAAACTGTTTTTAGAAAGTATTTTTAATTTCAGCAAGT | |
| | | TTGGTTACTTGTTGCATGACTCTTAACACAGCTGACTTTT | |
| | | TGTGTCAGTGCAATGTATATTTTTTGTCCTGTTATTAACT | |
| | | TGTAAGCCCTAGTAATGGCCAATTATTTGTACAGCAACAG | |
| | | AAGTAAATTGAAGATACTGGCTAAGACTGGATTGATTGTG | |
| | | GACTTTTATACTATATTGCAGAAACCAATATCTGTTTCTT | |
| | | GGTGGTTATGTAAAAGACCTGAAGAATTACTATCTAGTGT | |
| | | GCAGTCTGTGATATCTGAATGTTCATTGTATATTTGTCTC | |
| | | TGATGCAAAAAGGTAGAGTAACACAATTACAATACATGAT | |
| | | TAAATGCAATAGTCCAGGTACTTAAGTAATTTTTTTTTCA | |
| | | TTTCAAATAAATACCTATTTACCACCAAAAGAAAGAAAAA | |
| | | AAAAAAAAA | |
| ZFHX3 | NM_001164766.1 | CGCGGCCCGAGCGCCTCTTTTCGGGATTAAAAGCGCCGCC | 50 |
| | | AGCTCCCGCCGCCGCCGCCGTCGCCAGCAGCGCCGCTGCA | |
| | | GCCGCCGCCGCCGGAGAAGCAACCGCTGGGCGGTGAGATC | |
| | | CCCCTAGACATGCGGCTCGGGGGCGGGCAGCTGGTGTCAG | |
| | | AGGAGCTGATGAACCTGGGCGAGAGCTTCATCCAGACCAA | |
| | | CGACCCGTCGCTGAAGCTCTTCCAGTGCGCCGTCTGCAAC | |
| | | AAGTTCACGACGGACAACCTGGACATGCTGGGCCTGCACA | |
| | | TGAACGTGGAGCGCAGCCTGTCGGAGGACGAGTGGAAGGC | |
| | | GGTGATGGGGGACTCATACCAGTGCAAGCTCTGCCGCTAC | |
| | | AACACCCAGCTCAAGGCCAACTTCCAGCTGCACTGCAAGA | |
| | | CAGACAAGCACGTGCAGAAGTACCAGCTGGTGGCCCACAT | |
| | | CAAGGAGGGCGGCAAGGCCAACGAGTGGAGGCTCAAGTGT | |
| | | GTGGCCATCGGCAACCCCGTGCACCTCAAGTGCAACGCCT | |
| | | GTGACTACTACACCAACAGCCTGGAGAAGCTGCGGCTGCA | |
| | | CACGGTCAACTCCAGGCACGAGGCCAGCCTGAAGTTGTAC | |
| | | AAGCACCTGCAGCAGCATGAGAGTGGTGTAGAAGGTGAGA | |
| | | GCTGCTACTACCACTGCGTTCTGTGCAACTACTCCACCAA | |
| | | GGCCAAGCTCAACCTCATCCAGCATGTGCGCTCCATGAAG | |
| | | CACCAGCGAAGCGAGAGCCTGCGAAAGCTGCAGCGGCTGC | |
| | | AGAAGGGCCTTCCAGAGGAGGACGAGGACCTGGGGCAGAT | |
| | | CTTCACCATCCGCAGGTGCCCCTCCACGGACCCAGAAGAA | |
| | | GCCATTGAAGATGTTGAAGGACCCAGTGAAACAGCTGCTG | |
| | | ATCCAGAGGAGCTTGCTAAGGACCAAGAGGGCGGAGCATC | |
| | | GTCCAGCCAAGCAGAGAAGGAGCTGACAGATTCTCCTGCA | |
| | | ACCTCCAAACGCATCTCCTTCCCAGGTAGCTCAGAGTCTC | |
| | | CCCTCTCTTCGAAGCGACCAAAAACAGCTGAGGAGATCAA | |
| | | ACCGGAGCAGATGTACCAGTGTCCCTACTGCAAGTACAGT | |
| | | AATGCCGATGTCAACCGGCTCCGGGTGCATGCCATGACGC | |
| | | AGCACTCGGTGCAACCCATGCTTCGCTGCCCCCTGTGCCA | |
| | | GGACATGCTCAACAACAAGATCCACCTCCAGCTGCACCTC | |
| | | ACCCACCTCCACAGCGTGGCACCTGACTGCGTGGAGAAGC | |
| | | TCATTATGACGGTGACCACCCCTGAGATGGTGATGCCAAG | |
| | | CAGCATGTTCCTCCCAGCAGCTGTTCCAGATCGAGATGGG | |
| | | AATTCCAATTTGGAAGAGGCAGGAAAGCAGCCTGAAACCT | |
| | | CAGAGGATCTGGGAAAGAACATCTTGCCATCCGCAAGCAC | |
| | | AGAGCAAAGCGGAGATTTGAAACCATCCCCTGCTGACCCA | |
| | | GGCTCTGTGAGAGAAGACTCAGGCTTCATCTGCTGGAAGA | |
| | | AGGGGTGCAACCAGGTTTTCAAAACTTCTGCTGCCCTTCA | |
| | | GACGCATTTTAATGAAGTGCATGCCAAGAGGCCTCAGCTG | |
| | | CCGGTGTCAGATCGCCATGTGTACAAGTACCGCTGTAATC | |
| | | AGTGTAGCCTGGCCTTCAAGACCATTGAAAAGTTGCAGCT | |
| | | CCATTCTCAGTACCATGTGATCAGAGCTGCCACCATGTGC | |
| | | TGTCTTTGTCAGCGCAGTTTCCGAACTTTCCAGGCTCTGA | |
| | | AGAAGCACCTTGAGACAAGCCACCTGGAGCTGAGTGAGGC | |
| | | TGACATCCAACAGCTTTATGGTGGCCTGCTGGCCAATGGG | |
| | | GACCTCCTGGCAATGGGAGACCCCACTCTGGCAGAGGACC | |
| | | ATACCATAATTGTTGAGGAAGACAAGGAGGAAGAGAGTGA | |
| | | CTTGGAAGATAAACAGAGCCCAACGGGCAGTGACTCTGGG | |
| | | TCAGTACAAGAAGACTCGGGCTCAGAGCCAAAGAGAGCTC | |
| | | TGCCTTTCAGAAAAGGTCCCAATTTTACTATGGAAAAGTT | |
| | | CCTAGACCCTTCTCGCCCTTACAAGTGTACCGTCTGCAAG | |
| | | GAATCTTTCACTCAAAAGAATATCCTGCTAGTACACTACA | |
| | | ATTCTGTCTCCCACCTGCATAAGTTAAAGAGAGCCCTTCA | |
| | | AGAATCAGCAACCGGTCAGCCAGAACCCACCAGCAGCCCA | |
| | | GACAACAAACCTTTTAAGTGTAACACTTGTAATGTGGCCT | |
| | | ACAGCCAGAGTTCCACTCTGGAGATCCATATGAGGTCTGT | |
| | | GTTACATCAAACCAAGGCCCGGGCAGCCAAGCTGGAGGCT | |
| | | GCAAGTGGCAGCAGCAATGGGACTGGGAACAGCAGCAGTA | |
| | | TTTCCTTGAGCTCCTCCACGCCAAGTCCTGTGAGCACCAG | |
| | | TGGCAGTAACACCTTTACCACCTCCAATCCAAGCAGTGCT | |
| | | GGCATTGCTCCAAGCTCTAACTTACTAAGCCAAGTGCCCA | |
| | | CTGAGAGTGTAGGGATGCCACCCCTGGGGAATCCTATTGG | |
| | | TGCCAACATTGCTTCCCCTTCAGAGCCCAAAGAGGCCAAT | |
| | | CGGAAGAAACTGGCAGATATGATTGCATCCAGGCAGCAGC | |
| | | AACAACAGCAGCAGCAACAGCAACAACAACAACAACAACA | |
| | | ACAACAACAAGCACAAACGCTGGCCCAGGCCCAGGCTCAA | |
| | | GTTCAAGCTCACCTGCAGCAGGAGCTGCAGCAACAGGCTG | |
| | | CCCTGATCCAGTCTCAGCTGTTTAACCCCACCCTCCTTCC | |
| | | TCACTTCCCCATGACAACTGAGACCCTGCTGCAACTACAG | |
| | | CAGCAGCAGCACCTCCTCTTCCCTTTCTACATCCCCAGTG | |
| | | CTGAGTTCCAGCTTAACCCCGAGGTGAGCTTGCCAGTGAC | |
| | | CAGTGGGGCACTGACACTGACTGGGACAGGCCCAGGCCTG | |
| | | CTGGAAGATCTGAAGGCTCAGGTTCAGGTCCCACAGCAGA | |
| | | GCCATCAGCAGATCTTGCCGCAGCAGCAGCAGAACCAACT | |
| | | CTCTATAGCCCAGAGTCACTCTGCCCTCCTTCAGCCAAGC | |
| | | CAGCACCCCGAAAAGAAGAACAAATTGGTCATCAAAGAAA | |
| | | AGGAAAAAGAAAGCCAGAGAGAGAGGGACAGCGCCGAGGG | |
| | | GGGAGAGGGCAACACCGGTCCGAAGGAAACACTGCCAGAT | |
| | | GCCTTGAAGGCCAAAGAGAAGAAAGAGTTGGCACCAGGGG | |
| | | GTGGTTCTGAGCCTTCCATGCTCCCTCCACGCATTGCTTC | |
| | | AGATGCCAGAGGGAACGCCACCAAGGCCCTGCTGGAGAAC | |
| | | TTTGGCTTTGAGTTGGTCATCCAGTATAATGAGAACAAGC | |
| | | AGAAGGTGCAGAAAAAGAATGGGAAGACTGACCAGGGAGA | |
| | | GAACCTGGAAAAGCTCGAGTGTGACTCCTGCGGCAAGTTG | |
| | | TTTTCCAACATCTTGATTTTAAAGAGTCATCAAGAGCACG | |
| | | TTCATCAGAATTACTTTCCTTTCAAACAGCTCGAGAGGTT | |
| | | TGCCAAACAGTACAGAGACCACTACGATAAACTGTACCCA | |
| | | CTGAGGCCCCAGACCCCAGAGCCACCACCACCTCCCCCTC | |
| | | CACCCCCTCCACCCCCACTTCCGGCAGCGCCGCCTCAGCC | |
| | | GGCGTCCACACCAGCCATCCCCGCATCAGCCCCACCCATC | |
| | | ACCTCACCTACAATTGCACCGGCCCAGCCATCAGTGCCGC | |
| | | TCACCCAGCTCTCCATGCCGATGGAGCTGCCCATCTTCTC | |
| | | GCCGCTGATGATGCAGACGATGCCGCTGCAGACCTTGCCG | |
| | | GCTCAGCTACCCCCGCAGCTGGGACCTGTGGAGCCTCTGC | |
| | | CTGCGGACCTGGCCCAACTCTACCAGCATCAGCTCAATCC | |
| | | AACCCTGCTCCAGCAGCAGAACAAGAGGCCTCGCACCAGG | |
| | | ATCACAGATGATCAGCTCCGAGTCTTGCGGCAATATTTTG | |
| | | ACATTAACAACTCCCCCAGTGAAGAGCAAATAAAAGAGAT | |
| | | GGCAGACAAGTCCGGGTTGCCCCAGAAAGTGATCAAGCAC | |
| | | TGGTTCAGGAACACTCTCTTCAAAGAGAGGCAGCGTAACA | |
| | | AGGACTCCCCTTACAACTTCAGTAATCCTCCTATCACCAG | |
| | | CCTGGAGGAGCTCAAGATTGACTCCCGGCCCCCTTCGCCG | |
| | | GAACCTCCAAAGCAGGAGTACTGGGGAAGCAAGAGGTCTT | |
| | | CAAGAACAAGGTTTACGGACTACCAGCTGAGGGTCTTACA | |
| | | GGACTTCTTCGATGCCAATGCTTACCCAAAGGATGATGAA | |
| | | TTTGAGCAACTCTCTAATTTACTGAACCTTCCAACCCGAG | |
| | | TGATAGTGGTGTGGTTTCAGAATGCCCGACAGAAGGCCAG | |
| | | GAAGAATTATGAGAATCAGGGAGAGGGCAAAGATGGAGAG | |
| | | CGGCGTGAGCTTACAAATGATAGATACATTCGAACAAGCA | |
| | | ACTTGAACTACCAGTGCAAAAAATGTAGCCTGGTGTTTCA | |
| | | GCGCATCTTTGATCTCATCAAGCACCAGAAGAAGCTGTGT | |
| | | TACAAGGATGAGGATGAGGAGGGGCAGGACGACAGCCAAA | |
| | | ATGAGGATTCCATGGATGCCATGGAAATCCTGACGCCTAC | |
| | | CAGCTCATCCTGCAGTACCCCGATGCCCTCACAGGCTTAC | |
| | | AGCGCCCCAGCACCATCAGCCAATAATACAGCTTCCTCCG | |
| | | CTTTCTTGCAGCTTACAGCGGAGGCTGAGGAACTGGCCAC | |
| | | CTTCAATTCAAAAACAGAGGCAGGCGATGAGAAACCAAAG | |
| | | CTGGCGGAAGCTCCCAGTGCACAGCCAAACCAAACCCAAG | |
| | | AAAAGCAAGGACAACCAAAGCCAGAGCTGCAGCAGCAAGA | |
| | | GCAGCCCGAGCAGAAGACCAACACTCCCCAGCAGAAGCTC | |
| | | CCCCAGCTGGTGTCCCTGCCTTCGTTGCCACAGCCTCCTC | |
| | | CACAAGCGCCCCCTCCACAGTGCCCCTTACCCCAGTCGAG | |
| | | CCCCAGTCCTTCCCAGCTCTCCCACCTGCCCCTCAAGCCC | |
| | | CTCCACACATCAACTCCTCAACAGCTCGCAAACCTACCTC | |
| | | CTCAGCTAATCCCCTACCAGTGTGACCAGTGTAAGTTGGC | |
| | | ATTTCCGTCATTTGAGCACTGGCAGGAGCATCAGCAGCTC | |
| | | CACTTCCTGAGCGCGCAGAACCAGTTCATCCACCCCCAGT | |
| | | TTTTGGACAGGTCCCTGGATATGCCTTTCATGCTCTTTGA | |
| | | TCCCAGTAACCCACTCCTGGCCAGCCAGCTGCTCTCTGGG | |
| | | GCCATACCTCAGATTCCAGCAAGCTCAGCCACTTCTCCTT | |
| | | CAACTCCAACCTCCACAATGAACACTCTCAAGAGGAAGCT | |
| | | GGAGGAAAAGGCCAGTGCAAGCCCTGGCGAAAACGACAGT | |
| | | GGGACAGGAGGAGAAGAGCCTCAGAGAGACAAGCGTTTGA | |
| | | GAACAACCATCACACCGGAACAACTAGAAATTCTCTACCA | |
| | | GAAGTATCTACTGGATTCCAATCCGACTCGAAAGATGTTG | |
| | | GATCACATTGCACACGAGGTGGGCTTGAAGAAACGTGTGG | |
| | | TACAAGTCTGGTTTCAGAACACCCGAGCTCGGGAAAGGAA | |
| | | AGGACAGTTCCGGGCTGTAGGCCCAGCGCAGGCCCACAGG | |
| | | AGATGCCCTTTTTGCAGAGCGCTCTTCAAAGCCAAGACTG | |
| | | CTCTTGAGGCTCATATCCGGTCCCGTCACTGGCATGAAGC | |
| | | CAAGAGAGCTGGCTACAACCTAACTCTGTCTGCGATGCTC | |
| | | TTAGACTGTGATGGGGGACTCCAGATGAAAGGAGATATTT | |
| | | TTGACGGAACTAGCTTTTCCCACCTACCCCCAAGCAGTAG | |
| | | TGATGGTCAGGGTGTCCCCCTCTCACCTGTGAGTAAAACC | |
| | | ATGGAATTGTCACCCAGAACTCTTCTAAGCCCTTCCTCCA | |
| | | TTAAGGTGGAAGGGATTGAAGACTTTGAAAGCCCCTCCAT | |
| | | GTCCTCAGTTAATCTAAACTTTGACCAAACTAAGCTGGAC | |
| | | AACGATGACTGTTCCTCTGTCAACACAGCAATCACAGATA | |
| | | CCACAACTGGAGACGAGGGCAACGCAGATAACGACAGTGC | |
| | | AACGGGAATAGCAACTGAAACCAAATCCTCTTCTGCACCC | |
| | | AACGAAGGGTTGACCAAAGCGGCCATGATGGCAATGTCTG | |
| | | AGTATGAAGATCGGTTGTCATCTGGTCTGGTCAGCCCGGC | |
| | | CCCGAGCTTTTATAGCAAGGAATATGACAATGAAGGTACA | |
| | | GTGGACTACAGTGAAACCTCAAGCCTTGCAGATCCCTGCT | |
| | | CCCCGAGTCCTGGTGCGAGTGGATCTGCAGGCAAATCTGG | |
| | | TGACAGCGGAGATCGGCCTGGGCAGAAACGTTTTCGCACT | |
| | | CAAATGACCAATCTGCAGCTGAAGGTCCTCAAGTCATGCT | |
| | | TTAATGACTACAGGACACCCACTATGCTAGAATGTGAGGT | |
| | | CCTGGGCAATGACATTGGACTGCCAAAGAGAGTCGTTCAG | |
| | | GTCTGGTTCCAGAATGCCCGGGCAAAAGAAAAGAAGTCCA | |
| | | AGTTAAGCATGGCCAAGCATTTTGGTATAAACCAAACGAG | |
| | | TTATGAGGGACCCAAAACAGAGTGCACTTTGTGTGGCATC | |
| | | AAGTACAGCGCTCGGCTGTCTGTACGTGACCATATCTTTT | |
| | | CCCAACAGCATATCTCCAAAGTTAAAGACACCATTGGAAG | |
| | | CCAGCTGGACAAGGAGAAAGAATACTTTGACCCAGCCACC | |
| | | GTACGTCAGTTGATGGCTCAACAAGAGTTGGACCGGATTA | |
| | | AAAAGGCCAACGAGGTCCTTGGACTGGCAGCTCAGCAGCA | |
| | | AGGGATGTTTGACAACACCCCTCTTCAGGCCCTTAACCTT | |
| | | CCTACAGCATATCCAGCGCTCCAGGGCATTCCTCCTGTGT | |
| | | TGCTCCCGGGCCTCAACAGCCCCTCCTTGCCAGGCTTTAC | |
| | | TCCATCCAACACAGCTTTAACGTCTCCTAAGCCGAACTTG | |
| | | ATGGGTCTGCCCAGCACAACTGTTCCTTCCCCTGGCCTCC | |
| | | CCACTTCTGGATTACCAAATAAACCGTCCTCAGCGTCGCT | |
| | | GAGCTCCCCAACCCCAGCACAAGCCACGATGGCGATGGGC | |
| | | CCTCAGCAACCCCCCCAGCAGCAGCAGCAGCAGCAGCAAC | |
| | | CACAGGTGCAGCAGCCTCCCCCGCCGCCAGCAGCCCAGCC | |
| | | GCCACCCACACCACAGCTCCCACTGCAACAGCAGCAGCAA | |
| | | CGCAAGGACAAAGACAGTGAGAAAGTAAAGGAGAAGGAAA | |
| | | AGGCACACAAAGGGAAAGGGGAACCCCTGCCTGTCCCCAA | |
| | | GAAGGAGAAAGGAGAGGCCCCCACGGCAACTGCAGCCACG | |
| | | ATCTCAGCCCCGCTGCCCACCATGGAGTATGCGGTAGACC | |
| | | CTGCACAGCTGCAGGCCCTGCAGGCCGCGTTGACTTCGGA | |
| | | CCCCACAGCATTGCTCACAAGCCAGTTCCTTCCTTACTTT | |
| | | GTACCAGGCTTTTCTCCTTATTATGCTCCCCAGATCCCTG | |
| | | GCGCCCTGCAGAGCGGGTACCTGCAGCCTATGTATGGCAT | |
| | | GGAAGGCCTGTTCCCCTACAGCCCTGCACTGTCGCAGGCC | |
| | | CTGATGGGGCTGTCCCCAGGCTCCCTACTGCAGCAGTACC | |
| | | AGCAATACCAGCAGAGTCTGCAGGAGGCAATTCAGCAGCA | |
| | | GCAGCAGCGGCAACTACAGCAGCAGCAGCAGCAAAAAGTG | |
| | | CAGCAGCAGCAGCCCAAAGCAAGCCAAACCCCAGTCCCCC | |
| | | CCGGGGCTCCTTCCCCAGACAAAGACCCTGCCAAAGAATC | |
| | | CCCCAAACCAGAAGAACAGAAAAACACCCCCCGTGAGGTG | |
| | | TCCCCCCTCCTGCCGAAACTCCCTGAAGAGCCAGAAGCAG | |
| | | AAAGCAAAAGTGCGGACTCCCTCTACGACCCCTTCATTGT | |
| | | TCCAAAGGTGCAGTACAAGTTGGTCTGCCGCAAGTGCCAG | |
| | | GCGGGCTTCAGCGACGAGGAGGCAGCGAGGAGCCACCTGA | |
| | | AGTCCCTCTGCTTCTTCGGCCAGTCTGTGGTGAACCTGCA | |
| | | AGAGATGGTGCTTCACGTCCCCACCGGCGGCGGCGGCGGT | |
| | | GGCAGTGGCGGCGGCGGCGGCGGTGGCGGCGGCGGCGGCG | |
| | | GCGGCGGCTCGTACCACTGCCTGGCGTGCGAGAGCGCGCT | |
| | | CTGTGGGGAGGAAGCTCTGAGTCAACATCTCGAGTCGGCC | |
| | | TTGCACAAACACAGAACAATCACGAGAGCAGCAAGAAACG | |
| | | CCAAAGAGCACCCTAGTTTATTACCTCACTCTGCCTGCTT | |
| | | CCCCGATCCTAGCACCGCATCTACCTCGCAGTCTGCCGCT | |
| | | CACTCAAACGACAGCCCCCCTCCCCCGTCGGCCGCCGCCC | |
| | | CCTCCTCCGCTTCCCCCCACGCCTCCAGGAAGTCTTGGCC | |
| | | GCAAGTGGTCTCCCGGGCTTCGGCAGCGAAGCCCCCTTCT | |
| | | TTTCCTCCTCTCTCCTCATCTTCAACGGTTACCTCAAGTT | |
| | | CATGCAGCACCTCAGGGGTTCAGCCCTCGATGCCAACAGA | |
| | | CGACTATTCGGAGGAGTCTGACACGGATCTCAGCCAAAAG | |
| | | TCCGACGGACCGGCGAGCCCGGTGGAGGGTCCCAAAGACC | |
| | | CCAGCTGCCCCAAGGACAGTGGTCTGACCAGTGTAGGAAC | |
| | | GGACACCTTCAGATTGTAAGCTTTGAAGATGAACAATACA | |
| | | AACAAATGAATTTAAATACAAAAATTAATAACAAACCAAT | |
| | | TTCAAAAATAGACTAACTGCAATTCCAAAGCTTCTAACCA | |
| | | AAAAACAAAAAAAAAAAAAAAAAGAAAAAAAAGAAAAAGC | |
| | | GTGGGTTGTTTTCCCATATACCTATCTATGCCGGTGATTT | |
| | | TACATTCTTGTCTTTTTCTTTTCTTTTAATATTAAAAAAA | |
| | | AAAAAAAAGCCCTAACCCTGTTACATTGTGTCCTTTTGAA | |
| | | GGTACTATTGGTCTGGGAAACAGAAGTCCGCAGGGCCTCC | |
| | | CTAATGTCTTTGGAGCTTAAACCCCTTGTATATTTGCCCC | |
| | | TTTTCAATAAACGCCCCACGCTGATAGCACAGAGGAGCCC | |
| | | GGCATGCACTGTATGGGAAAGCAGTCCACCTTGTTACAGT | |
| | | TTTAAATTTCTTGCTATCTTAGCATTCAGATACCAATGGC | |
| | | TTGCTAAAAGAAAAAAAGAAATGTAATGTCTTTTTATTCT | |
| | | CAGGTCAATCGCTCACACTTTGTTTTCAGAATCATTGTTT | |
| | | TATATATTATTGTTTTTTCAGTTTTTTTTTTTTTTTTTGT | |
| | | TCCAGAAAAGATTTTTTGTTTTGTTAACTTAAAAATGGGC | |
| | | AGAAAGTATTCAAGAAAAACAATGTGAACTGCTTTAGCTT | |
| | | TCTGGGGATTTTTAAGGATAGCTTTTCTGCTGAAGCCAAT | |
| | | TTCAAGGGGAAAAGTTAAGCACTCCCACTTTCAAAAAAAA | |
| | | AAAAAAATAATAACCCACACACACAAAGAGTGTTGAGGAC | |
| | | TTGTAGCTTAAAAAAAATAAGTTTTAAAAACTGACTTTCT | |
| | | GTATTTATGATAGATATGACCATTTTTGGTGTTGAGTAGA | |
| | | TTGTTGCATTGGAAATGAACTGAAGCAGTATGGTAGATTT | |
| | | AAAAGGAAAAAAAAAAAAAAACCTTTTGTGTACATTTAGC | |
| | | TTTTTGTATGGTCCAGCTGACAGCTCCTCATTTGATGTTG | |
| | | TCTTGTTCATTCCTAGCAGATGATAGATTGCAATCCGTTG | |
| | | ATTCGCCTAAGCTTTTCTCCCCTTGTCCCTTAATTCCACT | |
| | | TTCTCTTTCTTGTCCCTTAATTCCACTTTCTCTTTCCTTC | |
| | | TCCCACCTCCCGTCCTATAATCTCCCACTTAAGGTAGCTG | |
| | | CCTTCATTTCTTAGAGGGAGCTGCAGAATTATTTTATAAA | |
| | | ACTAAAGAAAGAATTTCAAGGGATTCTAGGGGTCATTAGG | |
| | | ATCCTCACAGATTATTTTTGGTTGGGGAGTTGAAACTTTT | |
| | | TAAAGGCATATAATTCTAGTTACCTGTGTCTGTTAGCTTT | |
| | | GTGCATTTATTTTTTATTTATCCTTCTTTTGGCTTTTTTT | |
| | | TCTTTGTACCCCTTCTTTTCCTCCTTGTTTGGTAGGAGCT | |
| | | TCAAATATTCTTTTTTTTTCTATACTAAAGGATTTGTTTC | |
| | | CATTTGTGTAATTGGCTGTGTACTTTTCTTTTCTAAAAAA | |
| | | AGTTTTTGGTTAGGGATTTGGTTTTTGGTTTTGTGTTTGT | |
| | | TTTTTCTTTCCTCTCTCAGAAAAAAAAATTTCATGCTTTA | |
| | | AATAAAATCCAAAGACACACCCTTTCACTGCTGATGCAGA | |
| | | AAAAAGGGAAAGGGTTCTTGTTACTTGAGAATTTGTTTCT | |
| | | GATTTAAACAAACAAGACTTAGTTTAATAAAAGAAAGAGA | |
| | | AAAACAAAAGATTCCCAGGTTGTTATGTGCTTCTTCTGCA | |
| | | AGCAGAGAGGCAAATGTTAATGACAATTCCATATACCAAA | |
| | | AGACACATTTTTTACTTCAAAGTTTTGTCCTTGTGTTAGG | |
| | | CAGTCTGAGCAGCGAGTGATCCAGAGCGCAGCCAACAAAG | |
| | | CAGCAGATAGCAGTGTACAGAAAGCAAAAAAGGAACTGTA | |
| | | TGTGAGGCACTTGTTTCTGTTAATATCCATATTCCTGTTA | |
| | | ACACACACCCTTTCTCATGTAAAAAGAAAAATAAATAAAT | |
| | | GGTCTGAACTTTGAAAACTTTGTGCTGCTAAAACATAGAT | |
| | | TTTGGAGACAAATAAATAGATGCTTTGCTGTTTCACTTTC | |
| | | ATAGCTAAACATCAACAGAAACCATCTCCCCTTGCCCCCA | |
| | | AAGTGTGAAATCCTTCTTCCCTTCGTTTTCTTCCTTATGT | |
| | | TTCAAAAGGGAACTTTGAAGACTGTGAATACAGGTTCCAT | |
| | | TGGTCACCTTTCGGGCTTCTTTCCCCAGTGCTGAAGCCAC | |
| | | TCATCGACTTTGCAAAAGACTGGAGCATTCCAAGATCTGA | |
| | | AAATGGATTTTTTTTCTTTTTTTCTTTTTTAGCCGGGACT | |
| | | ATTTTATTTTTATGAATTTGTTTTTAGTTTAATGAAATAG | |
| | | TAGATCCTGAAATGTTGTACATATTTCTAACTAGGCTGAT | |
| | | GCACAGTGCAAATTCCTTTTTTAATTGTTTTTTTTAAGTA | |
| | | GAAATACTAAAGAAAGAATACCATCTAACTATTCATACCA | |
| | | GTATCCAGTTGTAGCATAAGGTGTCAAAAGCAAGTACGCA | |
| | | AAACATTTACTGTTTTAACAAGCTATTTCCTTTTAACAAG | |
| | | AAATCTTGTATTTCTTCCTGTGTTTGAGATGAACATTTTT | |
| | | AAATTTTAAAGTTGTACAGTTTTTTGTTTTCCATTATTTT | |
| | | ATCTTGTTTGTAACTCTATGAAATATATATATATATATTT | |
| | | TTTGCCATTTAACTGTTGTATGTTACTCTGTGTCTGTACC | |
| | | ATATAGAAAAAAAATTGTTTTTGTTTTTGGTTCTCTATGT | |
| | | GATATCAGTTAACAATGTAACACTAGCTTTACCTGTCAAA | |
| | | TTCTGCTAGGTCTTCTCTGAAAACGTTGTTTTTAAAAATG | |
| | | ATATTGCTTGGTAATAGTGCAATTTCTATCCTTTTCCCTC | |
| | | CCCCCTCAACTTTTAAGTTCTTTTCTTTATAATTTTGCTG | |
| | | CCCCCTCCCTGATGGTTTGGGTTTTTGTTTTTGTTTTTGT | |
| | | TTTTTTTTTTCATGGAGCTACTATGCCATCCTCCCTCTGT | |
| | | GAGGCAGAGTGACTGTCAGTGTTTTGTTATGCCATGCCTT | |
| | | GAGCTGTGGGTGTTTGGCGACAATAAGGTGGTTGAATAGA | |
| | | TTGGCTGAGCACACTTCCACCCACCTAGTGTTCTCAGAGG | |
| | | GGTTATGTGATTGTTTCAACCTGGAGTGGGTTGCACCCTT | |
| | | AATGCTTTCCTCTGCAACTAAACCGCCCACATATATGTTC | |
| | | ATTGAAAAAAGTAAGAATAATTCTCAGCACTAACCCAGAA | |
| | | GTAGCAAAGCAGTCAGTGATGGTGAACATTAGAGGTCAAA | |
| | | CATGAGTTAGATGTTTGTGGGCTGACAGCCATCGTGGCTA | |
| | | TGACCAGTACTATTTACAAAGCATGAATTCACTACAATGC | |
| | | TCAACTGTTTGTTTAGCTTTATCTCACTTGGGGAATTTAT | |
| | | TCCTGTCTGCTGCATTGTAGGTAGCTGGGTAGGATATATT | |
| | | TCCACTTGCTTTTTAAATTAGTTCTTCACCTCCATTGACA | |
| | | CTCGTTTTTTGGTTTTCTCCCTATAGTGTGGGTTGGTGCT | |
| | | AGACACCAGTCTGACCCACAGAATGGGAGTTATTTCATCC | |
| | | ATCTTTCCTCCATCCTTCCAAAAACCACATATCTACACAA | |
| | | GGAAAAATTTAATACATCTAGGAATTTTTTTTTTAATTAC | |
| | | AAGCTATTTAAAGAGATGAATGTGGCCAAAGTTTTACACA | |
| | | ATTGAAAATAAAGTAAAACAGACGGCATGTGTTTAAACCT | |
| | | GAGTTTATCAGGCATGGCAGGAAGTTGCAGGAGAGAGAGG | |
| | | CAGTGACCCAAGCCAGTGCACTTGATGTTCATGGACATAT | |
| | | ATTTTTTTTAAATAATAAATTAAAACATTTTAAATAGAAG | |
| | | CATAAATTGAGTTGTTTGTTGGCGCTGAGATACTGCCCAC | |
| | | TGTGAAACAAAGCTTTGACTAGTTTTTTGTTTGTTTACTT | |
| | | TCTTCAGGGGGGAGGGGGGCAAGTTTGGGTAGGAAAGAAA | |
| | | GCATAAATGAACGTGACCCTGAGGTGAAGAGGTATATGAA | |
| | | CAGCCTTTGCAATGTACAAAAAGAAAAAAAAACAAAAAAC | |
| | | AACAAAAAAAATAGAGCAAGTGAAACCAAAAATGATGTTC | |
| | | TTGGTGTTTTTCTATAATGTAGTCTTGTTAGCTTTTTTGT | |
| | | TACTGTAACAATGCTGATCTCGAACTGTACCAAAATACAT | |
| | | GGAGACTAACAAACAGAACCACATGGAACTTTCAAACTGA | |
| | | AAAAAAAATTTGTCACAAAAACTTTGTTGTCATAGTTAAG | |
| | | TTGATTGTAGATGGTAATTGAATATACTCCTTTGAAAATA | |
| | | TTTCATCAAGTATGTTTCCTGCTCATTGTGATACATTAAA | |
| | | AAAAAAATATGAGCAAAA | |
| ZXDC | NM_001040653.3 | GGGCGCGGGCAGCTCTGCGTCCGAAGCTGCTCCGACGCCG | 51 |
| | | TCGCTGGGACCAAGATGGACCTCCCGGCGCTGCTCCCCGC | |
| | | CCCGACTGCGCGCGGAGGGCAACATGGCGGCGGCCCCGGC | |
| | | CCGCTCCGCCGAGCCCCAGCGCCGCTCGGCGCGAGCCCCG | |
| | | CGCGCCGCCGCCTGCTACTGGTGCGGGGCCCTGAAGATGG | |
| | | CGGGCCCGGGGCGCGGCCCGGGGAGGCCTCCGGGCCAAGC | |
| | | CCGCCGCCCGCCGAGGACGACAGCGACGGCGACTCTTTCT | |
| | | TGGTGCTGCTGGAAGTGCCGCACGGCGGCGCTGCCGCCGA | |
| | | GGCTGCCGGATCACAGGAGGCCGAGCCTGGCTCCCGTGTC | |
| | | AACCTGGCGAGCCGCCCCGAGCAGGGCCCCAGCGGCCCGG | |
| | | CCGCCCCCCCCGGCCCTGGCGTAGCCCCGGCGGGCGCCGT | |
| | | CACCATCAGCAGCCAGGACCTGCTGGTGCGTCTCGACCGC | |
| | | GGCGTCCTCGCGCTGTCTGCGCCGCCCGGCCCCGCAACCG | |
| | | CGGGCGCCGCCGCTCCCCGCCGCGCGCCCCAGGCCTCCGG | |
| | | CCCCAGCACGCCCGGCTACCGCTGCCCCGAGCCGCAGTGC | |
| | | GCGCTGGCCTTCGCCAAGAAGCACCAGCTCAAGGTGCACC | |
| | | TGCTCACGCACGGCGGCGGTCAGGGCCGGCGGCCCTTCAA | |
| | | GTGCCCACTGGAGGGCTGTGGTTGGGCCTTCACAACGTCC | |
| | | TACAAGCTCAAGCGGCACCTGCAGTCGCACGACAAGCTGC | |
| | | GGCCCTTCGGCTGTCCAGTGGGCGGCTGTGGCAAGAAGTT | |
| | | CACTACGGTCTATAACCTCAAGGCGCACATGAAGGGCCAC | |
| | | GAGCAGGAGAGCCTGTTCAAGTGCGAGGTGTGCGCCGAGC | |
| | | GCTTCCCCACGCACGCCAAGCTCAGCTCCCACCAGCGCAG | |
| | | CCACTTCGAGCCCGAGCGCCCTTACAAGTGTGACTTTCCC | |
| | | GGCTGTGAGAAGACATTTATCACAGTGAGTGCCCTGTTTT | |
| | | CCCATAACCGAGCCCACTTCAGGGAACAAGAGCTCTTTTC | |
| | | CTGCTCCTTTCCTGGGTGCAGCAAGCAGTATGATAAAGCC | |
| | | TGTCGGCTGAAAATTCACCTGCGGAGCCATACAGGTGAAA | |
| | | GACCATTTATTTGTGACTCTGACAGCTGTGGCTGGACCTT | |
| | | CACCAGCATGTCCAAACTTCTAAGGCACAGAAGGAAACAT | |
| | | GACGATGACCGGAGGTTTACCTGCCCTGTCGAGGGCTGTG | |
| | | GGAAATCATTCACCAGAGCAGAGCATCTGAAAGGCCACAG | |
| | | CATAACCCACCTAGGCACAAAGCCGTTCGAGTGTCCTGTG | |
| | | GAAGGATGTTGCGCGAGGTTCTCCGCTCGTAGCAGTCTGT | |
| | | ACATTCACTCTAAGAAACACGTGCAGGATGTGGGTGCTCC | |
| | | GAAAAGCCGTTGCCCAGTTTCTACCTGCAACAGACTCTTC | |
| | | ACCTCCAAGCACAGCATGAAGGCGCACATGGTCAGACAGC | |
| | | ACAGCCGGCGCCAAGATCTCTTACCTCAGCTAGAAGCTCC | |
| | | GAGTTCTCTTACTCCCAGCAGTGAACTCAGCAGCCCAGGC | |
| | | CAAAGTGAGCTCACTAACATGGATCTTGCTGCACTCTTCT | |
| | | CTGACACACCTGCCAATGCTAGTGGTTCTGCAGGTGGGTC | |
| | | GGATGAGGCTCTGAACTCCGGAATCCTGACTATTGACGTC | |
| | | ACTTCTGTGAGCTCCTCTCTGGGAGGGAACCTCCCTGCTA | |
| | | ATAATAGCTCCCTAGGGCCGATGGAACCCCTGGTCCTGGT | |
| | | GGCCCACAGTGATATTCCCCCAAGCCTGGACAGCCCTCTG | |
| | | GTTCTCGGGACAGCAGCCACGGTTCTGCAGCAGGGCAGCT | |
| | | TCAGTGTGGATGACGTGCAGACTGTGAGTGCAGGAGCATT | |
| | | AGGCTGTCTGGTGGCTCTGCCCATGAAGAACTTGAGTGAC | |
| | | GACCCACTGGCTTTGACCTCCAATAGTAACTTAGCAGCAC | |
| | | ATATCACCACACCGACCTCTTCGAGCACCCCCCGAGAAAA | |
| | | TGCCAGTGTCCCGGAACTGCTGGCTCCAATCAAGGTGGAG | |
| | | CCGGACTCGCCTTCTCGCCCAGGAGCAGTTGGGCAGCAGG | |
| | | AAGGAAGCCATGGGCTGCCCCAGTCCACGTTGCCCAGTCC | |
| | | AGCAGAGCAGCACGGTGCCCAGGACACAGAGCTCAGTGCA | |
| | | GGCACTGGCAACTTCTATTTGGTATGAAGCACTCTATTCA | |
| | | GTCACCACCATATAGGTCACTTCTCTCATACTCGGTCTTG | |
| | | AGGATATTCTGGATTAATCCTTTCTATGCAGACGTTTCTG | |
| | | GTTTACAAAAGGACGCAGCCCTGGACTACAAGTCTGGAAC | |
| | | TGACAAGTTCTTATGACCTTGACAAATCACCTTAACCCAT | |
| | | CTGAGCCTTAAATTCTCATTTATTTCCTGCATAAGGAGAT | |
| | | TTGGCTAAATGCTTTCTGAGGTCCTTTGGAGTCCTGTGGC | |
| | | TCCATGGTAATGTGCTCCTTTCCTTGAAGATTGGGGGTTT | |
| | | TGTAATGTTGAGATACTTTGCCTCTATGCTTGTCAGCTCA | |
| | | TGACCAGTCCTAGAAGAGGAGTCGAGACATAAGCCACCTT | |
| | | CAGAGGTTCAATGGAAACTTTAAAACCATACCAAACTCTT | |
| | | TTTTAAAATTAGAATTAACAAGAAAAAAAAAAAGGGTGGG | |
| | | GTTTATGAGCCTTAGTTCTTGGAGGATTATAAGAGTACTT | |
| | | CCCCAGTTTTGAGGCTGGACAGTTAATATACTTTATATCA | |
| | | ATTATACATTTAATATAATTTAATTTAAAATAATTTAAAG | |
| | | ATTCTTAGGAGATAGTCTGACTTTCCTGACCTAGATGGGA | |
| | | ATGATCAGATAGGGATTTTTTTTGTGGCACAGGCTAAATT | |
| | | TGATGGTGACATTTATATTGTTGAGAATGTTACATCTTAT | |
| | | TTTACCACAACTTTTAAAAAATGTTACATCTTTTGCAGTA | |
| | | GGATCAGTTGTGAGGCACATAGTAGCTGAGGCTCCATGGA | |
| | | GCCACCTTTCATTTCTTTCAGTCAGAGAGGAGGACAGTCT | |
| | | CTGTCTCTGCATTTCTGGTGTCTTGCTTGTCGGTGGCAGA | |
| | | GCCATGCTTGCCGGCATTTGCTTAGGCGGCCATAGTAGTT | |
| | | GCTAAGTGTACAGGTGACTGGGCAGGGATGGGAGGTGGCC | |
| | | ACAGGTCAGAGACAAGTGCTCAGTCAGTCCCTGGTGCCAG | |
| | | GACTGTGTGCCTCGGTGCCTTGGGAAATGGAAGCTCCCTG | |
| | | GTGCAGCTGCAGCTGTGGGTGGAGGTAGAGAAGCCAGCAA | |
| | | GACCTTGGTCTTAACCCCGTGTTCATTTTCTTGCTAGCTG | |
| | | TGTGACGTTGGGCTACCTCGCTTCTCTGAGTACAAATGGT | |
| | | GTGTGGTGAATGGGTCCCAGGTATGCTACGAGCTTTGAGG | |
| | | GCTGCTCTTTTTCTCTTCATAGCGATAAGTGTTAAACTGT | |
| | | CTTTCTTAGGAAACGTTCACAGACTTGCAACAGCTGATGT | |
| | | CCTCTGAGTACTGTCTGACTCCCTCAGGCAAGTTCCTGAA | |
| | | TTCAGTACCATCATTATTATTTTTGTGTAAGACTTTGACA | |
| | | AAGTATAGCCCCTGCCACCAGAGCAGCCTGTACAGTGGGT | |
| | | CTCTAAGGTGGGACCTGCCCCGGGCCTGCCATGCACGTGT | |
| | | GTGAAACAGCGTGAAAAGTGTCGCGGTAAGGTGACCCTGG | |
| | | GTTACCCAGGCAAGGCTCGGTGTTTGTTTCAGAAAGCAGA | |
| | | GAAGTATGTAATTGATTTTAAAAGTTTCTGTTTAAAATAT | |
| | | TTGGCTATGTTTTAGACTATGAAGGAATGAACTTTGCTTC | |
| | | TCTGGATAAGAAAGTCACATACATTGTTCCAGCTCCAAGT | |
| | | TTGTTCGGCCCTCGCCACAAGTGGATGTAGCGTTTGGCCC | |
| | | TTTGTGTGCCTTGCTGGTGACTCTGGTTTTGGGAGCTCGG | |
| | | ATATGTCCCAGAAGCAGGCTTATGGCACTTCTGTAGCTCC | |
| | | CTTGCTACCCTTCCTTTGTGTCTAGATAAGTGACTGACAT | |
| | | GCTTTTCTTTGGTCTCAGGAAAGTGGGGGCTCAGCAAGAA | |
| | | CTGATTACCGAGCCATTCAACTAGCCAAGGAAAAAAAGCA | |
| | | GAGAGGAGCGGGGAGCAATGCAGGTGAGGCCGTGTGTGCT | |
| | | GCAGCCGGACGAGCAAGGGCCTGAGGGTTCTCTGTCACTG | |
| | | TTACTGGCAGAAGAAACACAGCAGGTGTTTCTGTGCTCTT | |
| | | GGTTTTACTTTTCTGTTCAGAATACCCTTTTATCAACTCC | |
| | | TTAGTTTTATTTGAACTTAAGGGAAAAAATTAGTAACAAA | |
| | | ATTCCCAGCATCAGTATGAACATATTTTATTTGCCTAAAC | |
| | | AAGCTTTGTGAAAGTTAAGCGTTCAAACACCAGTGTCAGT | |
| | | TACCTGGAAGGCTACTAAGGTAAATAAGCAAAGCAGGCCA | |
| | | GTTGTCAGGAAAGCAGAGATTGTGCCTGGTGCTGAATGGC | |
| | | CTTGGGGCCTGATCTTGGCATGGCAGAGACCTGGGGACTG | |
| | | CCACTGTCCCCAGGTACGTGTACATGGAGCCAAACTGTGT | |
| | | GTCCTGTGGCATTGTCAGAGTTATGTTGAAATCTTATTTG | |
| | | AAAATGTTAGCAACTTACTTGCATTTTTAAAGACCAAACA | |
| | | AGAGCTGGTAACCTATGGCCTCAAGCATCTGTCCTTCCTA | |
| | | AAAATGGAATAGTGGGATGTAGTGCTTAATGGAAACTGCT | |
| | | AAATCTTTTTCTAAAAACTAACAGTGGATTTTTAAAATAT | |
| | | ATTGTTTTTTGTGTATTTCATTTGTCCTTTGTATTTATCT | |
| | | AAAAGGGTTGATATGATTTTATATCTTGCTCTCTATTCCT | |
| | | AATAGTATTATGACTTCTTATTTAAAATAAATAACAATTG | |
| | | CCGGTTTTCTGTTAAAAAAAAAAAAA | |
| ZZZ3 | NM_015534.4 | GTTGGCAGAGCAGTTGTCCTGGATGGCGGAGCCTTGGGTT | 52 |
| | | CCGGGGGCCTGGGACCTGCAACTCTTTCTACAAGATATCA | |
| | | AGTTATTCTAGTACAACCATATAAATAAATAATACCTGAA | |
| | | GTCTCAGTGTAACATGGACAATTAACAGTGATGACAGATA | |
| | | AATACAGACGCATGGGGATCAAATACTAGGCAAAACGCTT | |
| | | TTTAAAAGTGTATCAGGCTTTTAAGAAACACTGCAGGATC | |
| | | CTGTCTATCTTAATGCTGATAGAGCTCAGCTAAAAATTTA | |
| | | GGAGGTTCTAGTATTCTTCATGGCTGAAGCTGAGAGAGTC | |
| | | TGAAACCCTGATGCTTAAGCTCCATTCTAGATCATAGCTC | |
| | | CAACTCCTTCAGGATATAAGGAAAAGAGATTATATTTCCA | |
| | | CAATGATAGATCTTTGGTTGTACAGGTTTCCCAATGAGTG | |
| | | GATCATGATGACCGTATTGTAGGGACTTGCCATAGTATGG | |
| | | CTGCTTCCCGATCTACTCGTGTTACAAGATCAACAGTGGG | |
| | | GTTAAACGGCTTGGATGAATCTTTTTGTGGTAGAACTTTA | |
| | | AGGAATCGTAGCATTGCGCATCCTGAAGAAATCTCTTCTA | |
| | | ATTCTCAAGTACGATCAAGATCACCAAAGAAGAGACCAGA | |
| | | GCCTGTGCCAATTCAGAAAGGAAATAATAATGGGAGAACC | |
| | | ACTGATTTAAAACAGCAGAGTACCCGAGAATCATGGGTAA | |
| | | GCCCTAGGAAAAGAGGACTTTCTTCTTCAGAAAAGGATAA | |
| | | CATAGAAAGGCAGGCTATAGAAAATTGTGAGAGAAGGCAA | |
| | | ACAGAACCTGTTTCACCAGTTTTAAAAAGAATTAAGCGTT | |
| | | GTCTTAGATCTGAAGCACCAAACAGTTCAGAAGAAGATTC | |
| | | TCCTATAAAATCAGACAAGGAGTCAGTAGAACAGAGGAGT | |
| | | ACAGTAGTGGACAATGATGCAGATTTTCAAGGGACTAAAC | |
| | | GAGCTTGTCGATGTCTTATACTGGATGATTGTGAGAAAAG | |
| | | GGAAATTAAAAAGGTGAATGTCAGTGAGGAAGGGCCACTT | |
| | | AATTCTGCAGTAGTTGAAGAAATCACAGGCTATTTGGCTG | |
| | | TCAATGGTGTTGATGACAGTGATTCAGCTGTTATAAACTG | |
| | | TGATGACTGTCAGCCTGATGGGAACACTAAACAAAATAGC | |
| | | ATTGGTTCCTATGTGTTACAGGAAAAATCAGTAGCTGAAA | |
| | | ATGGGGATACGGATACCCAAACTTCAATGTTCCTTGATAG | |
| | | TAGGAAGGAGGACAGTTATATAGACCATAAGGTGCCTTGC | |
| | | ACAGATTCACAAGTGCAGGTCAAGTTGGAGGACCACAAAA | |
| | | TAGTAACTGCCTGCTTGCCTGTGGAACATGTTAATCAGCT | |
| | | GACTACTGAGCCAGCTACAGGGCCCTTTTCTGAAACTCAG | |
| | | TCATCTTTAAGGGATTCTGAGGAGGAAGTAGATGTGGTGG | |
| | | GAGATAGCAGTGCCTCAAAAGAGCAGTGTAAAGAAAACAC | |
| | | CAATAACGAACTGGACACAAGTCTTGAGAGTATGCCAGCC | |
| | | TCCGGAGAACCTGAACCATCTCCTGTTCTAGACTGTGTTT | |
| | | CAGCTCAAATGATGTCTTTATCAGAACCTCAAGAACATCG | |
| | | TTATACTCTGAGAACCTCACCACGAAGGGCAGCCCCTACC | |
| | | AGAGGTAGTCCCACTAAAAACAGTTCTCCTTACAGAGAAA | |
| | | ATGGACAATTTGAGGAGAATAATCTTAGTCCTAATGAAAC | |
| | | AAATGCAACTGTTAGTGATAATGTAAGTCAATCTCCTACA | |
| | | AATCCTGGTGAAATTTCTCAAAATGAAAAAGGGATATGTT | |
| | | GTGACTCTCAAAATAATGGAAGTGAAGGAGTAAGTAAACC | |
| | | ACCCTCAGAGGCAAGACTCAATATTGGACATTTGCCATCT | |
| | | GCCAAAGAGAGTGCCAGTCAGCACATTACAGAAGAGGAAG | |
| | | ATGATGATCCTGATGTTTATTACTTTGAATCAGATCATGT | |
| | | GGCACTGAAACACAACAAAGATTATCAGAGACTATTACAG | |
| | | ACGATTGCTGTACTCGAGGCTCAGCGTTCTCAAGCAGTCC | |
| | | AAGACCTTGAAAGTTTAGGCAGGCACCAGAGAGAAGCACT | |
| | | GAAAAATCCCATTGGATTTGTGGAAAAACTCCAGAAGAAG | |
| | | GCTGATATTGGGCTTCCATATCCACAGAGAGTTGTTCAAT | |
| | | TGCCTGAGATCGTATGGGACCAATATACCCATAGCCTTGG | |
| | | GAATTTTGAAAGAGAATTTAAAAATCGTAAAAGACATACT | |
| | | AGAAGAGTTAAGCTAGTTTTTGATAAAGTAGGTTTACCTG | |
| | | CTAGACCAAAAAGTCCTTTAGATCCTAAGAAGGATGGAGA | |
| | | GTCCCTTTCATATTCTATGTTGCCTTTGAGTGATGGTCCA | |
| | | GAAGGCTCAAGCAGTCGTCCTCAGATGATAAGAGGACGCT | |
| | | TGTGTGATGATACCAAACCTGAAACATTTAACCAGTTGTG | |
| | | GACTGTTGAAGAACAGAAAAAGCTGGAACAGCTACTCATC | |
| | | AAATACCCTCCTGAAGAAGTAGAATCTCGACGCTGGCAGA | |
| | | AGATAGCAGATGAATTGGGCAACAGGACAGCAAAACAGGT | |
| | | TGCCAGCCGAGTACAGAAGTATTTCATAAAGCTAACTAAA | |
| | | GCTGGCATTCCAGTACCAGGCAGAACACCAAACTTATATA | |
| | | TATACTCCAAAAAGTCTTCAACAAGCAGACGACAGCACCC | |
| | | TCTTAATAAGCATCTCTTTAAGCCTTCCACTTTCATGACT | |
| | | TCACATGAACCGCCAGTGTATATGGATGAAGATGATGACC | |
| | | GATCTTGTTTTCATAGCCACATGAACACTGCTGTTGAAGA | |
| | | TGCATCAGATGACGAAAGTATTCCTATCATGTATAGGAAT | |
| | | TTACCTGAATATAAAGAACTATTACAGTTTAAAAAGTTAA | |
| | | AGAAGCAGAAACTTCAGCAAATGCAAGCTGAAAGTGGATT | |
| | | TGTGCAACATGTGGGCTTTAAGTGTGATAACTGTGGCATA | |
| | | GAACCCATCCAGGGTGTTCGGTGGCATTGCCAGGATTGTC | |
| | | CTCCAGAAATGTCTTTGGATTTCTGTGATTCTTGTTCAGA | |
| | | CTGTCTACATGAAACAGATATTCACAAGGAAGATCACCAA | |
| | | TTAGAACCTATTTATAGGTCAGAGACATTCTTAGACAGAG | |
| | | ACTACTGTGTGTCTCAGGGCACCAGTTACAATTACCTTGA | |
| | | CCCAAACTACTTTCCAGCAAACAGATGACATGGAAGAGAA | |
| | | CATCATTTACTAGTCCTCTTCAACACATAGCAATGGTATC | |
| | | ATTGTTAATTATGTGCACAGTTTGGAAAGATTCTCTGCTT | |
| | | TCCCAGAAATGACACTCACAGCATGAGAGCTTCCTGAGTG | |
| | | TTCTCGTCAAGTACAGCTCTGCACCGTTGTGGCTCTAGAT | |
| | | CACTGTTCAGCAGCTGAACATTCCTGGTGAGCAAAGGTTT | |
| | | CCCTGGTGAATTTTTCACCACTGCGTTTTAGGTGGTGATC | |
| | | TTAAATGGGTGAGATGGAACGAGAGCACACATTAAAGAGA | |
| | | GAGTAAATTCCAAAGGTTTCAAAGAACTTGGTCATAAATA | |
| | | TGATAATGAGAAGACAAAGTATTTATATTAAAACAGTTTA | |
| | | GTAGCCTTCAGTTTTGTGAAAATAGTTTTCAGCACAGAAA | |
| | | CTGACTTCTTTAGACAAAGTTTTAACCAATGATGGTGTTT | |
| | | GCTTCTAGGATATACACTTTAAAAGAACTCACTGTCCCAG | |
| | | TGGTGGTCATTGATGGCCTTTAGTAAATTGGAGCTGCTTA | |
| | | ATCATATTGATATCTAATTTCTTTTAACCACAATGAATTG | |
| | | TCCTTAATTACCAACAGTGAAGCACTACAGGAGGCAACTG | |
| | | TGGCATTGCTTCCTTAACCAGCTCATGGTGTGTGAATGTT | |
| | | ATAAAATTGTCACTCAGATATATTTTTTAAATGTAATGTT | |
| | | ATATAAGATGATCATGTGATGTGTACAAACTATGGTGAAA | |
| | | AGTGCCAGTGGTAGTAACTGTGTAAAGTTTCTAATTCACA | |
| | | ACATTAATTCCTTTAAAATACACAGCCTTCTGCCTCTGTA | |
| | | TTTGGAGTTGTCAGTACAACTCATCAAAGAAAACTGCCTA | |
| | | ATATAAAAATCATATATATGGTAATAATTTCCCTCTTTTG | |
| | | TAGTCTGCACAAGATCCATAAAAGATTGTATTTTTATTAC | |
| | | TATTTAAACAAGTGATTAAATTTAGTCTGCACAGTGAGCA | |
| | | AGGGTTCACATGCATTCTTTTATACTGCTGGATTTTGTTG | |
| | | TGCATCATTTAAAACATTTTGTATGTTTCTTCTTATCTGT | |
| | | GTATACAGTATGTTCTTGAATGATGTTCATTTGTCAGGAG | |
| | | AACTGTGAGAAATAAACTATGTGGATACTGTCTGTTTATA | |
| | | TTAAAAGAAAAAAAAAAAAAAAAA | |

The 51 GEP-NEN biomarkers include: AKAP8L (A kinase (PRKA) anchor protein 8-like), APLP2 (amyloid beta (A4) precursor-like protein 2), ARAF1 (v-raf murine sarcoma 3611 viral oncogene homolog), ATP6V1H (ATPase, H+ transporting, lysosomal 50/57 kDa, VI subunit H), BNIP3L (BCL2/adenovirus E1B 19 kDa interacting protein 3-like), BRAF (v-raf murine sarcoma viral oncogene homolog BI), C21ORF7 (chromosome 21 open reading frame 7), CD59 (CD59 molecule, complement regulatory protein), COMMD9 (COMM domain containing 9), CTGF (connective tissue growth factor), ENPP4 (ectonucleotide pyrophosphatase/phosphodiesterase 4), FAM131A (family with sequence similarity 131, member A, transcript variant 2), FLJ 10357 (Rho guanine nucleotide exchange factor (GEF) 40 (ARHGEF40), FZD7 (frizzled homolog 7 (Drosophila)), GLT8D1 (glycosyltransferase 8 domain containing 1, transcript variant 3), HDAC9 (histone deacetylase 9, transcript variant 6), HSF2 (heat shock transcription factor 2, transcript variant 1), Ki-67 (antigen identified by monoclonal antibody Ki-67), KRAS (v-Ki-ras2 Kirsten rat sarcoma viral oncogene homolog), LEO1 (Pafl/RNA polymerase II complex component homolog (S. cerevisiae)), MORF4L2 (mortality factor 4 like 2, transcript variant 1), NAPILI (nucleosome assembly protein 1-like 1), NOL3 (nucleolar protein 3 (apoptosis repressor with CARD domain), transcript variant 3), NUDT3 (nudix (nucleoside diphosphate linked moiety X)-type motif 3), OAZ2 (ornithine decarboxylase antizyme 2), PANK2 (pantothenate kinase 2), PHF21A (PHD finger protein 21A, transcript variant 1), PKD1 (polycystic kidney disease 1 (autosomal dominant), transcript variant 2), PLD3 (phospholipase D family, member 3, transcript variant 1), PNMA2 (paraneoplastic antigen MA2), PQBP1 (polyglutamine binding protein 1, transcript variant 2), RAF1 (v-raf-1 murine leukemia viral oncogene homolog 1), RNF41 (ring finger protein 41, transcript variant 4), RSF1 (remodeling and spacing factor 1), RTN2 (reticulon 2, transcript variant 1), SMARCD3 (SWI/SNF related, matrix associated, actin dependent regulator of chromatin, subfamily d, member 3, transcript variant 3), SPATA7 (spermatogenesis associated 7, transcript variant 2), SST1 (somatostatin receptor 1), SST3 (somatostatin receptor 3), SST4 (somatostatin receptor 4), SST5 (somatostatin receptor 5, transcript variant 1), TECPR2 (tectonin beta-propeller repeat containing 2, transcript variant 2), TPH1 (tryptophan hydroxylase 1), TRMT112 (tRNA methyltransferase 11-2 homolog (S. cerevisiae)), VMAT1 (solute carrier family 18 (vesicular monoamine), member 1), VMAT 2 (solute carrier family 18 (vesicular monoamine), member 2), VPS13C (vacuolar protein sorting 13 homolog C (S. cerevisiae), transcript variant 2B), WDFY3 (WD repeat and FYVE domain containing 3), ZFHX3 (zinc finger homeobox 3, transcript variant B), ZXDC (zinc finger C, transcript variant 2), and ZZZ3 (zinc finger, ZZ-type containing 3), including gene products typically human gene products, including transcripts, mRNA, cDNA, coding sequences, proteins and polypeptides, as well as polynucleotides (nucleic acids) encoding the proteins and polypeptides, including naturally occurring variants, e.g., allelic variants, splice variants, transcript variants, and single nucleotide polymorphism (SNP) variants. For example, the biomarkers include polynucleotides, proteins, and polypeptides having the sequences disclosed herein, and naturally occurring variants thereof.

The housekeeping gene used to normalize expression of the 51 marker genes is the human ALG9 (asparagine-linked glycosylation 9, alpha-1,2-mannosyltransferase homolog).

Of these 51 differentially expressed biomarker genes, 38 biomarker genes are useful for the generation of mathematically-derived expression level scores for diagnosing, monitoring, and/or prognosticating the presence of GEP-NEN and/or different states of GEP-NENs. These 38 GEP-NEN biomarkers include: PNMA2, NAPILI, FZD7, SLC18A2/VMAT2, NOL3, SSTR5, TPH1, RAF1, RSF1, SSTR3, SSTR1, CD59, ARAF, APLP2, KRAS, MORF4L2, TRMT112, MKI67/KI67, SSTR4, CTGF, SPATA7, ZFHX3, PHF21A, SLC18A1/VMAT1, ZZZ3, TECPR2, ATP6V1H, OAZ2, PANK2, PLD3, PQBP1, RNF41, SMARCD3, BNIP3L, WDFY3, COMMD9, BRAF, and GLT8D1.

Of the 38 biomarker genes useful for the generation of a mathematically-derived expression level score for diagnosing, monitoring, and/or prognosticating the presence of GEP-NENs, at least 22 biomarker genes may be needed to generate an adequate classifier. These at least 22 biomarker genes include PNMA2, NAPILI, FZD7, SLC18A2, NOL3, SSTR5, TPH1, RAF1, RSF1, SSTR3, SSTR1, CD59, ARAF, APLP2, KRAS, MORF4L2, TRMT112, MKI67, SSTR4, CTGF, SPATA7, and ZFHX3.

The ALG9 biomarkers/housekeeping genes include human ALG9 gene products, including natural variants, e.g., allelic variants, and homologs and analogs thereof. In one example, the ALG9 biomarker/housekeeping gene is a polynucleotide having the nucleotide sequence set forth in SEQ ID NO: 1 (referenced at NM_024740.2), or containing a protein-coding portion thereof, a natural variant thereof, or a protein encoded by such a polynucleotide.

The AKAP8L biomarkers include human AKAP8L gene products, including natural variants, e.g., allelic variants, and homologs and analogs thereof. In one example, the AKAP8L biomarker is a polynucleotide having the nucleotide sequence set forth in SEQ ID NO: 2 (referenced at NM_014371.3), or containing a protein-coding portion thereof, a natural variant thereof, or a protein encoded by such a polynucleotide.

The APLP2 biomarkers include human APLP2 gene products, including natural variants, e.g., allelic variants, and homologs and analogs thereof. In one example, the APLP2 biomarker is a polynucleotide having the nucleotide sequence set forth in SEQ ID NO: 3 (referenced at NM_001142276.1) or containing a protein-coding portion thereof, a natural variant thereof, or a protein encoded by such a polynucleotide.

The ARAF1 biomarkers include human ARAF1 gene products, including natural variants, e.g., allelic variants, and homologs and analogs thereof. In one example, the ARAF1 biomarker is a polynucleotide having the nucleotide sequence set forth in SEQ ID NO: 4 (referenced at NM_001654.4), or containing a protein-coding portion thereof, a natural variant thereof, or a protein encoded by such a polynucleotide.

The ATP6V1H biomarkers include human ATP6V1H gene products, including natural variants, e.g., allelic variants, and homologs and analogs thereof. In one example, the ATP6V1H biomarker is a polynucleotide having the nucleotide sequence set forth in SEQ ID NO: 5 (referenced at NM_015941.3), or containing a protein-coding portion thereof, a natural variant thereof, or a protein encoded by such a polynucleotide.

The BNIP3L biomarkers include human BNIP3L gene products, including natural variants, e.g., allelic variants, and homologs and analogs thereof. In one example, the BNIP3L biomarker is a polynucleotide having the nucleotide sequence set forth in SEQ ID NO: 6 (referenced at NM_004331.2), or containing a protein-coding portion thereof, a natural variant thereof, or a protein encoded by such a polynucleotide.

The BRAF biomarkers include BRAF gene products, including natural variants, e.g., allelic variants, and homologs and analogs thereof. In one example, the BRAF biomarker is a polynucleotide having the nucleotide sequence set forth in SEQ ID NO: 7 (referenced at NM_004333.4), or containing a protein-coding portion thereof, a natural variant thereof, or a protein encoded by such a polynucleotide.

The C21ORF7 biomarkers include C21ORF7 gene products, including natural variants, e.g., allelic variants, and homologs and analogs thereof. In one example, the C21ORF7 biomarker is a polynucleotide having the nucleotide sequence set forth in SEQ ID NO: 8 (referenced at NM_020152.3), or containing a protein-coding portion thereof, a natural variant thereof, or a protein encoded by such a polynucleotide.

The CD59 biomarkers include CD59 gene products, including natural variants, e.g., allelic variants, and homologs and analogs thereof. In one example, the CD59 biomarker is a polynucleotide having the nucleotide sequence set forth in SEQ ID NO: 9 (referenced at NM_203331.2), or containing a protein-coding portion thereof, a natural variant thereof, or a protein encoded by such a polynucleotide.

The COMMD9 biomarkers include COMMD9 gene products, including natural variants, e.g., allelic variants, and homologs and analogs thereof. In one example, the COMMD9 biomarker is a polynucleotide having the nucleotide sequence set forth in SEQ ID NO: 10 (referenced at NM_001101653.1), or containing a protein-coding portion thereof, a natural variant thereof, or a protein encoded by such a polynucleotide.

The CTGF biomarkers include CTGF gene products, including natural variants, e.g., allelic variants, and homologs and analogs thereof. In one example, the CTGF biomarker is a polynucleotide having the nucleotide sequence set forth in SEQ ID NO: 11 (referenced at NM_001901.2), or containing a protein-coding portion thereof, a natural variant thereof, or a protein encoded by such a polynucleotide.

The ENPP4 biomarkers include ENPP4 gene products, including natural variants, e.g., allelic variants, and homologs and analogs thereof. In one example, the ENPP4 biomarker is a polynucleotide having the nucleotide sequence set forth in SEQ ID NO: 12 (referenced at NM_014936.4), or containing a protein-coding portion thereof, a natural variant thereof, or a protein encoded by such a polynucleotide.

The FAM131A biomarkers include FAM131A gene products, including natural variants, e.g., allelic variants, and homologs and analogs thereof. In one example, the FAM131A biomarker is a polynucleotide having the nucleotide sequence set forth in SEQ ID NO: 13 (referenced at NM_001171093.1), or containing a protein-coding portion thereof, a natural variant thereof, or a protein encoded by such a polynucleotide.

The FLJ1035 biomarkers include FLJ1035 gene products, including natural variants, e.g., allelic variants, and homologs and analogs thereof. In one example, the FLJ1035 biomarker is a polynucleotide having the nucleotide sequence set forth in SEQ ID NO: 14 (referenced at NM_018071.4), or containing a protein-coding portion thereof, a natural variant thereof, or a protein encoded by such a polynucleotide.

The FZD7 biomarkers include FZD7 gene products, including natural variants, e.g., allelic variants, and homologs and analogs thereof. In one example, the FZD7 biomarker is a polynucleotide having the nucleotide sequence set forth in SEQ ID NO: 15 (referenced at NM_003507.1), or containing a protein-coding portion thereof, a natural variant thereof, or a protein encoded by such a polynucleotide.

The GLT8D1 biomarkers include GLT8D1 gene products, including natural variants, e.g., allelic variants, and homologs and analogs thereof. In one example, the GLT8D1 biomarker is a polynucleotide having the nucleotide sequence set forth in SEQ ID NO: 16 (referenced at NM_001010983.2), or containing a protein-coding portion thereof, a natural variant thereof, or a protein encoded by such a polynucleotide.

The HDAC9 biomarkers include HDAC9 gene products, including natural variants, e.g., allelic variants, and homologs and analogs thereof. In one example, the HDAC9 biomarker is a polynucleotide having the nucleotide sequence set forth in SEQ ID NO: 17 (referenced at NM_001204144.1), or containing a protein-coding portion thereof, a natural variant thereof, or a protein encoded by such a polynucleotide.

The HSF2 biomarkers include HSF2 gene products, including natural variants, e.g., allelic variants, and homologs and analogs thereof. In one example, the HSF2 biomarker is a polynucleotide having the nucleotide sequence set forth in SEQ ID NO: 18 (referenced at NM_004506.3), or containing a protein-coding portion thereof, a natural variant thereof, or a protein encoded by such a polynucleotide.

The Ki-67 biomarkers include Ki-67 gene products, including natural variants, e.g., allelic variants, and homologs and analogs thereof. In one example, the Ki-67 biomarker is a polynucleotide having the nucleotide sequence set forth in SEQ ID NO: 19 (referenced at NM_001145966.1), or containing a protein-coding portion thereof, a natural variant thereof, or a protein encoded by such a polynucleotide.

The KRAS biomarkers include KRAS gene products, including natural variants, e.g., allelic variants, and homologs and analogs thereof. In one example, the KRAS biomarker is a polynucleotide having the nucleotide sequence set forth in SEQ ID NO: 20 (referenced at NM_004985.4), or containing a protein-coding portion thereof, a natural variant thereof, or a protein encoded by such a polynucleotide.

The LEO1 biomarkers include LEO1 gene products, including natural variants, e.g., allelic variants, and homologs and analogs thereof. In one example, the LEO1 biomarker is a polynucleotide having the nucleotide sequence set forth in SEQ ID NO: 21 (referenced at NM_138792.3), or containing a protein-coding portion thereof, a natural variant thereof, or a protein encoded by such a polynucleotide.

The MORF4L2 biomarkers include MORF4L2 gene products, including natural variants, e.g., allelic variants, and homologs and analogs thereof. In one example, the MORF4L2 biomarker is a polynucleotide having the nucleotide sequence set forth in SEQ ID NO: 22 (referenced at NM_001142418.1), or containing a protein-coding portion thereof, a natural variant thereof, or a protein encoded by such a polynucleotide.

The NAP1L1 biomarkers include NAPILI gene products, including natural variants, e.g., allelic variants, and homologs and analogs thereof. In one example, the NAPILI biomarker is a polynucleotide having the nucleotide sequence set forth in SEQ ID NO: 23 (referenced at NM_139207.2), or containing a protein-coding portion thereof, a natural variant thereof, or a protein encoded by such a polynucleotide.

The NOL3 biomarkers include NOL3 gene products, including natural variants, e.g., allelic variants, and homologs and analogs thereof. In one example, the NOL3 biomarker is a polynucleotide having the nucleotide sequence set forth in SEQ ID NO: 24 (referenced at NM_001185057.2), or containing a protein-coding portion thereof, a natural variant thereof, or a protein encoded by such a polynucleotide.

The NUDT3 biomarkers include NUDT3 gene products, including natural variants, e.g., allelic variants, and homologs and analogs thereof. In one example, the NUDT3 biomarker is a polynucleotide having the nucleotide sequence set forth in SEQ ID NO: 25 (referenced at NM_006703.3), or containing a protein-coding portion thereof, a natural variant thereof, or a protein encoded by such a polynucleotide.

The OAZ2 biomarkers include OAZ2 gene products, including natural variants, e.g., allelic variants, and homologs and analogs thereof. In one example, the OAZ2 biomarker is a polynucleotide having the nucleotide sequence set forth in SEQ ID NO: 26 (referenced at NM_002537.3), or containing a protein-coding portion thereof, a natural variant thereof, or a protein encoded by such a polynucleotide.

The PANK2 biomarkers include PANK2 gene products, including natural variants, e.g., allelic variants, and homologs and analogs thereof. In one example, the PANK2 biomarker is a polynucleotide having the nucleotide sequence set forth in SEQ ID NO: 27 (referenced at NM_024960.4), or containing a protein-coding portion thereof, a natural variant thereof, or a protein encoded by such a polynucleotide.

The PHF21A biomarkers include PHF21A gene products, including natural variants, e.g., allelic variants, and homologs and analogs thereof. In one example, the PHF21A biomarker is a polynucleotide having the nucleotide sequence set forth in SEQ ID NO: 28 (referenced at NM_001101802.1), or containing a protein-coding portion thereof, a natural variant thereof, or a protein encoded by such a polynucleotide.

The PKD1 biomarkers include PKD1 gene products, including natural variants, e.g., allelic variants, and homologs and analogs thereof. In one example, the PKD1 biomarker is a polynucleotide having the nucleotide sequence set forth in SEQ ID NO: 29 (referenced at NM_000296.3), or containing a protein-coding portion thereof, a natural variant thereof, or a protein encoded by such a polynucleotide.

The PLD3 biomarkers include PLD3 gene products, including natural variants, e.g., allelic variants, and homologs and analogs thereof. In one example, the PLD3 biomarker is a polynucleotide having the nucleotide sequence set forth in SEQ ID NO: 30 (referenced at NM_001031696.3), or containing a protein-coding portion thereof, a natural variant thereof, or a protein encoded by such a polynucleotide.

The PNMA2 biomarkers include PNMA2 gene products, including natural variants, e.g., allelic variants, and homologs and analogs thereof. In one example, the PNMA2 biomarker is a polynucleotide having the nucleotide sequence set forth in SEQ ID NO: 31 (referenced at NM_007257.5), or containing a protein-coding portion thereof, a natural variant thereof, or a protein encoded by such a polynucleotide.

The PQBP1 biomarkers include PQBP1 gene products, including natural variants, e.g., allelic variants, and homologs and analogs thereof. In one example, the PQBP1 biomarker is a polynucleotide having the nucleotide sequence set forth in SEQ ID NO: 32 (referenced at NM_001032381.1), or containing a protein-coding portion thereof, a natural variant thereof, or a protein encoded by such a polynucleotide.

The RAF1 biomarkers include RAF1 gene products, including natural variants, e.g., allelic variants, and homologs and analogs thereof. In one example, the RAF1 biomarker is a polynucleotide having the nucleotide sequence set forth in SEQ ID NO: 33 (referenced at NM_002880.3), or containing a protein-coding portion thereof, a natural variant thereof, or a protein encoded by such a polynucleotide.

The RNF41 biomarkers include RNF41 gene products, including natural variants, e.g., allelic variants, and homologs and analogs thereof. In one example, the RNF41 biomarker is a polynucleotide having the nucleotide sequence set forth in SEQ ID NO: 34 (referenced at NM_001242826.1), or containing a protein-coding portion thereof, a natural variant thereof, or a protein encoded by such a polynucleotide.

The RSF1 biomarkers include RSF1 gene products, including natural variants, e.g., allelic variants, and homologs and analogs thereof. In one example, the RSF1 biomarker is a polynucleotide having the nucleotide sequence set forth in SEQ ID NO: 35 (referenced at NM_016578.3), or containing a protein-coding portion thereof, a natural variant thereof, or a protein encoded by such a polynucleotide.

The RTN2 biomarkers include RTN2 gene products, including natural variants, e.g., allelic variants, and homologs and analogs thereof. In one example, the RTN2 biomarker is a polynucleotide having the nucleotide sequence set forth in SEQ ID NO: 36 (referenced at NM_005619.4), or containing a protein-coding portion thereof, a natural variant thereof, or a protein encoded by such a polynucleotide.

The SMARCD3 biomarkers include SMARCD3 gene products, including natural variants, e.g., allelic variants, and homologs and analogs thereof. In one example, the SMARCD3 biomarker is a polynucleotide having the nucleotide sequence set forth in SEQ ID NO: 37 (referenced at NM_001003801.1), or containing a protein-coding portion thereof, a natural variant thereof, or a protein encoded by such a polynucleotide.

The SPATA7 biomarkers include SPATA7 gene products, including natural variants, e.g., allelic variants, and homologs and analogs thereof. In one example, the SPATA7 biomarker is a polynucleotide having the nucleotide sequence set forth in SEQ ID NO: 38 (referenced at NM_001040428.3), or containing a protein-coding portion thereof, a natural variant thereof, or a protein encoded by such a polynucleotide.

The SSTR1 biomarkers include SSTR1 gene products, including natural variants, e.g., allelic variants, and homologs and analogs thereof. In one example, the SSTR1 biomarker is a polynucleotide having the nucleotide sequence set forth in SEQ ID NO: 39 (referenced at NM_001049.2), or containing a protein-coding portion thereof, a natural variant thereof, or a protein encoded by such a polynucleotide.

The SSTR3 biomarkers include SSTR3 gene products, including natural variants, e.g., allelic variants, and homologs and analogs thereof. In one example, the SSTR3 biomarker is a polynucleotide having the nucleotide sequence set forth in SEQ ID NO: 40 (referenced at NM_001051.4), or containing a protein-coding portion thereof, a natural variant thereof, or a protein encoded by such a polynucleotide.

The SST4 biomarkers include SST4 gene products, including natural variants, e.g., allelic variants, and homologs and analogs thereof. In one example, the SST4 biomarker is a polynucleotide having the nucleotide sequence set forth in SEQ ID NO: 41 (referenced at NM_001052.2), or containing a protein-coding portion thereof, a natural variant thereof, or a protein encoded by such a polynucleotide.

The SST5 biomarkers include SST5 gene products, including natural variants, e.g., allelic variants, and homologs and analogs thereof. In one example, the SST5 biomarker is a polynucleotide having the nucleotide sequence set forth in SEQ ID NO: 42 (referenced at NM_001053.3), or containing a protein-coding portion thereof, a natural variant thereof, or a protein encoded by such a polynucleotide.

The TECPR2 biomarkers include TECPR2 gene products, including natural variants, e.g., allelic variants, and homologs and analogs thereof. In one example, the TECPR2 biomarker is a polynucleotide having the nucleotide sequence set forth in SEQ ID NO: 43 (referenced at NM_001172631.1), or containing a protein-coding portion thereof, a natural variant thereof, or a protein encoded by such a polynucleotide.

The TPH1 biomarkers include TPH1 gene products, including natural variants, e.g., allelic variants, and homologs and analogs thereof. In one example, the TPH1 biomarker is a polynucleotide having the nucleotide sequence set forth in SEQ ID NO: 44 (referenced at NM_004179.2), or containing a protein-coding portion thereof, a natural variant thereof, or a protein encoded by such a polynucleotide.

The TRMT112 biomarkers include TRMT112 gene products, including natural variants, e.g., allelic variants, and homologs and analogs thereof. In one example, the TRMT112 biomarker is a polynucleotide having the nucleotide sequence set forth in SEQ ID NO: 45 (referenced at NM_016404.2), or containing a protein-coding portion thereof, a natural variant thereof, or a protein encoded by such a polynucleotide.

The VMAT1 biomarkers include VMAT1 gene products, including natural variants, e.g., allelic variants, and homologs and analogs thereof. In one example, the VMAT1 biomarker is a polynucleotide having the nucleotide sequence set forth in SEQ ID NO: 46 (referenced at NM_003053.3), or containing a protein-coding portion thereof, a natural variant thereof, or a protein encoded by such a polynucleotide.

The VMAT2 biomarkers include VMAT2 gene products, including natural variants, e.g., allelic variants, and homologs and analogs thereof. In one example, the VMAT2 biomarker is a polynucleotide having the nucleotide sequence set forth in SEQ ID NO: 47 (referenced at NM_003054.4), or containing a protein-coding portion thereof, a natural variant thereof, or a protein encoded by such a polynucleotide.

The VPS13C biomarkers include VPS13C gene products, including natural variants, e.g., allelic variants, and homologs and analogs thereof. In one example, the VPS13C biomarker is a polynucleotide having the nucleotide sequence set forth in SEQ ID NO: 48 (referenced at NM_001018088.2), or containing a protein-coding portion thereof, a natural variant thereof, or a protein encoded by such a polynucleotide.

The WDFY3 biomarkers include WDFY3 gene products, including natural variants, e.g., allelic variants, and homologs and analogs thereof. In one example, the WDFY3 biomarker is a polynucleotide having the nucleotide sequence set forth in SEQ ID NO: 49 (referenced at NM_014991.4), or containing a protein-coding portion thereof, a natural variant thereof, or a protein encoded by such a polynucleotide.

The ZFHX3 biomarkers include ZFHX3 gene products, including natural variants, e.g., allelic variants, and homologs and analogs thereof. In one example, the ZFHX3 biomarker is a polynucleotide having the nucleotide sequence set forth in SEQ ID NO: 50 (referenced at NM_001164766.1), or containing a protein-coding portion thereof, a natural variant thereof, or a protein encoded by such a polynucleotide.

The ZXDC biomarkers include ZXDC gene products, including natural variants, e.g., allelic variants, and homologs and analogs thereof. In one example, the ZXDC biomarker is a polynucleotide having the nucleotide sequence set forth in SEQ ID NO: 51 (referenced at NM_001040653.3), or containing a protein-coding portion thereof, a natural variant thereof, or a protein encoded by such a polynucleotide.

The ZZZ3 biomarkers include ZZZ3 gene products, including natural variants, e.g., allelic variants, and homologs and analogs thereof. In one example, the ZZZ3 biomarker is a polynucleotide having the nucleotide sequence set forth in SEQ ID NO: 52 (referenced at NM_015534.4), or containing a protein-coding portion thereof, a natural variant thereof, or a protein encoded by such a polynucleotide.

In some embodiments, the panel of polynucleotides further includes one or more polynucleotide able to specifically hybridize to “housekeeping,” or reference genes, for example, genes for which differences in expression is known or not expected to correlate with differences in the variables analyzed, for example, with the presence or absence of GEP-NEN or other neoplastic disease, differentiation of various GEP-NEN sub-types, metastasis, mucosal or other tissue types, prognostic indications, and/or other phenotype, prediction, or outcome. In some aspects, expression levels of such housekeeping genes are detected and used as an overall expression level standards, such as to normalize expression data obtained for GEP-NEN biomarkers across various samples.

Housekeeping genes are well known in the art. Typically, the housekeeping genes include one or more genes characterized as particularly appropriate for analyzing GEP-NEN samples, such as ALG9. See Kidd M, et al., “GeneChip, geNorm and Gastrointestinal tumors: novel reference genes for real-time PCR.” Physiol Genomics 2007; 30:363-70. In the current application, ALG9 is the housekeeping gene of choice.

The present invention provides methods, compositions, and systems, for the detection of the GEP-NEN biomarkers and for identifying, isolating, and enriching tumors and cells that express the GEP-NEN biomarkers. For example, provided are agents, sets of agents, and systems for detecting the GEP-NEN biomarkers and methods for use of the same, including for diagnostic and prognostic uses.

In one embodiment, the agents are proteins, polynucleotides or other molecules which specifically bind to or specifically hybridize to the GEP-NEN biomarkers. The agents include polynucleotides, such as probes and primers, e.g. sense and antisense PCR primers, having identity or complementarity to the polynucleotide biomarkers, such as mRNA, or proteins, such as antibodies, which specifically bind to such biomarkers. Sets and kits containing the agents, such as agents specifically hybridizing to or binding the panel of biomarkers, also are provided.

Thus, the systems, e.g., microarrays, sets of polynucleotides, and kits, provided herein include those with nucleic acid molecules, typically DNA oligonucleotides, such as primers and probes, the length of which typically varies between 15 bases and several kilo bases, such as between 20 bases and 1 kilobase, between 40 and 100 bases, and between 50 and 80 nucleotides or between 20 and 80 nucleotides. In one aspect, most (i.e. at least 60% of) nucleic acid molecules of a nucleotide microarray, kit, or other system, are capable of hybridizing to GEP-NEN biomarkers.

In one example, systems containing polynucleotides that specifically hybridize to the biomarkers, e.g., nucleic acid microarrays, are provided to detect and measure changes in expression levels and determine expression profiles of the biomarkers according to the provided methods. Among such systems, e.g., microarrays, are those comprising polynucleotides able to hybridize to at least as at least 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 80, 85, 90, 95, or 100 or more biomarkers, such as to at least 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51, and/or all of the following sets of biomarkers:
PNMA2, NAPILI, FZD7, SLC18A2/VMAT2, NOL3, SSTR5, TPH1, RAF1, RSF1, SSTR3, SSTR1, CD59, ARAF, APLP2, KRAS, MORF4L2, TRMT112, MKI67/KI67, SSTR4, CTGF, SPATA7, ZFHX3, PHF21A, SLC18A1/VMAT1, ZZZ3, TECPR2, ATP6V1H, OAZ2, PANK2, PLD3, PQBP1, RNF41, SMARCD3, BNIP3L, WDFY3, COMMD9, BRAF, and GLT8D1 gene products;
In some aspects, at least 60%, or at least 70%, at least 80%, or more, of the nucleic acid molecules of the system, e.g., microarray, are able to hybridize to biomarkers in the panel of biomarkers. In one example, probes immobilized on such nucleotide microarrays comprise at least 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 80, 85, 90, 95, or 100 or more biomarkers, such as to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51, or more nucleic acid molecules able to hybridize to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 80, 85, 90, 95, or 100 or more biomarkers, such as to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51, or more of the biomarkers, where each of the nucleic acid molecules is capable of specifically hybridizing to a different one of the biomarkers, such that at least that many different biomarkers can be bound.

In one example, the remaining nucleic acid molecules, such as 40% or at most 40% of the nucleic acid molecules on the microarray or in the set of polynucleotides are able to hybridize to a set of reference genes or a set of normalization genes (such as housekeeping genes), for example, for normalization in order to reduce systemic bias. Systemic bias results in variation by inter-array differences in overall performance, which can be due to for example inconsistencies in array fabrication, staining and scanning, and variation between labeled RNA samples, which can be due for example to variations in purity. Systemic bias can be introduced during the handling of the sample in a microarray experiment. To reduce systemic bias, the determined RNA levels are preferably corrected for background non-specific hybridization and normalized.

The use of such reference probes is advantageous but not mandatory. In one embodiment a set of polynucleotides or system, e.g., microarray, is provided wherein at least 90% of the nucleic acid sequences are able to hybridize to the GEP-NEN biomarkers; further embodiments include such systems and sets in which at least 95% or even 100% of the polynucleotides hybridize to the biomarkers.

Disclosed herein are exemplary suitable polynucleotides, such as PCR primers. Other nucleic acid probes and primers, able to hybridize to different regions of the biomarkers are of course also suitable for use in connection with the provided systems, kits and methods.

The present invention provides methods for detecting and quantifying the biomarkers, including detecting the presence, absence, amount or relative amount, such as expression levels or expression profile of the biomarkers. Typically, the methods are nucleic acid based methods, for example, measuring the presence, amount or expression levels of biomarker mRNA expression. Such methods typically are carried out by contacting polynucleotide agents to biological samples, such as test samples and normal and reference samples, for example, to quantify expression levels of nucleic acid biomarkers (e.g., mRNA) in the samples.

Detection and analysis of biomarkers according to the provided embodiments can be performed with any suitable method known in the art. For example, where the biomarkers are RNA biomarkers, RNA detection and quantification methods are used.

Exemplary methods for quantifying or detecting nucleic acid expression levels, e.g., mRNA expression, are well known, and include northern blotting and in situ hybridization (Parker and Barnes, Methods in Molecular Biology 106:247-283, 1999); RNAse protection assays (Hod, Biotechniques 13:852-854, 1992); and quantitative or semi-quantitative reverse transcription polymerase chain reaction (RT-PCR) (Weis et al., Trends in Genetics 8:263-264, 1992), representative methods for sequencing-based gene expression analysis include Serial Analysis of Gene Expression (SAGE), and gene expression analysis by massively parallel signature sequencing (MPSS).

Therefore, in one embodiment, expression of the biomarker or biomarker panel includes RNA expression; the methods include determining levels of RNA of the biomarkers, such as RNA obtained from and/or present in a sample of a patient, and performing analysis, diagnosis, or predictive determinations based upon the RNA expression levels determined for the biomarkers or panel of biomarkers.

RNA samples can be processed in numerous ways, as is known to those in the art. Several methods are well known for isolation of RNA from samples, including guanidinium thiocyanate-phenol-chloroform extraction, which may be carried out using the TRIZOL® reagent, a proprietary formulation (see Chomczynski P, Sacchi N (2006). “The single-step method of RNA isolation by acid guanidinium thiocyanate-phenol-chloroform extraction: twenty-something years on”. Nat Protoc 1 (2): 581-5). In this method, TRIZOL® is used to extract RNA and DNA; chloroform and centrifugation are used to separate RNA from other nucleic acids, followed by a series of washes with ethanol for cleanup of the RNA sample.

The RNA samples can be freshly prepared from samples e.g., cells or tissues at the moment of harvesting; alternatively, they can be prepared from samples that stored at −70° C. until processed for sample preparation. Alternatively, tissues or cell samples can be stored under and/or subjected to other conditions known in the art to preserve the quality of the RNA, including fixation for example with formalin or similar agent; and incubation with RNase inhibitors such as RNASIN® (ribonuclease inhibitors) or RNASECURE™ (RNase inactivation reagents); aqueous solutions such as RNALATER® (RNA stabilization solutions), Hepes-Glutamic acid buffer mediated Organic solvent Protection Effect (HOPE®), and RCL2® (formalin-free tissue fixatives); and non-aqueous solutions such as Universal Molecular Fixative (Sakura Finetek USA Inc.). A chaotropic nucleic acid isolation lysis buffer (Boom method, Boom et al. J Clin Microbiol. 1990; 28:495-503) may also be used for RNA isolation.

In one embodiment, RNA is isolated from buffy coat by incubating samples with TRIZOL®, followed by RNA clean-up. RNA is dissolved in diethyl pyrocarbonate water and measured spectrophotometrically, and an aliquot analyzed on a Bioanalyzer (Agilent Technologies, Palo Alto, CA) to assess the quality of the RNA (Kidd M, et al. “The role of genetic markers—NAP 1L1, MAGE-D2, and MTA1—in defining small-intestinal carcinoid neoplasia,” Ann Surg Oncol 2006; 13 (2): 253-62). In another embodiment, RNA is isolated from plasma using the QIAamp RNA Blood Mini Kit; in some cases, this method allows better detection by real-time PCR of significantly more housekeeping genes from plasma compared to the TRIZOL® approach. In another embodiment, RNA is isolated directly from whole blood, for example, using the QIAamp RNA Blood Mini Kit in a similar manner.

Methods for isolating RNA from fixed, paraffin-embedded tissues as the RNA source are well-known and generally include mRNA isolation, purification, primer extension and amplification (for example: T. E. Godfrey et al, Molec. Diagnostics 2:84-91 [2000]; K. Specht et al., Am. J. Pathol. 158:419-29 [2001]). In one example, RNA is extracted from a sample such as a blood sample using the QIAamp RNA Blood Mini Kit RNA. Typically, RNA is extracted from tissue, followed by removal of protein and DNA and analysis of RNA concentration. An RNA repair and/or amplification step may be included, such as a step for reverse transcription of RNA for RT-PCR.

Expression levels or amounts of the RNA biomarkers may be determined or quantified by any method known in the art, for example, by quantifying RNA expression relative to housekeeping gene or with relation to RNA levels of other genes measured at the same time. Methods to determine RNA levels of genes are known to a skilled person and include, but are not limited to, Northern blotting, (quantitative) PCR, and microarray analysis.

RNA biomarkers can be reverse transcribed to produce cDNA and the methods of the present invention can include detecting and quantifying that produced cDNA. In some embodiments, the cDNA is detected by forming a complex with a labeled probe. In some embodiments, the RNA biomarkers are directed detected by forming a complex with a labeled probe or primer.

Northern blotting may be performed for quantification of RNA of a specific biomarker gene or gene product, by hybridizing a labeled probe that specifically interacts with the RNA, following separation of RNA by gel electrophoresis. Probes are for example labeled with radioactive isotopes or chemiluminescent substrates. Quantification of the labeled probe that has interacted with said nucleic acid expression product serves as a measure for determining the level of expression. The determined level of expression can be normalized for differences in the total amounts of nucleic acid expression products between two separate samples with for instance an internal or external calibrator by comparing the level of expression of a gene that is known not to differ in expression level between samples or by adding a known quantity of RNA before determining the expression levels.

For RT-PCR, biomarker RNA is reverse transcribed into cDNA. Reverse transcriptase polymerase chain reaction (RT-PCR) is, for example, performed using specific primers that hybridize to an RNA sequence of interest and a reverse transcriptase enzyme. Furthermore, RT-PCR can be performed with random primers, such as for instance random hexamers or decamers which hybridize randomly along the RNA, or oligo d (T) which hybridizes to the poly(A) tail of mRNA, and reverse transcriptase enzyme.

In some embodiments, RNA expression levels of the biomarkers in a sample, such as one from a patient suffering from or suspected of suffering from GEP-NEN or associated symptom or syndrome, are determined using quantitative methods such as by real-time rt-PCR (qPCR) or microarray analysis. In some embodiments, quantitative Polymerase Chain Reaction (QPCR) is used to quantify the level of expression of nucleic acids. In one aspect, detection and determining expression levels of the biomarkers is carried out using RT-PCR, GeneChip analysis, quantitative real-time PCR (Q RT-PCR), or carcinoid tissue microarray (TMA) immunostaining/quantitation, for example, to compare biomarker RNA, e.g., mRNA, or other expression product, levels in different sample populations, characterize patterns of gene expression, to discriminate between closely related mRNAs, and to analyze RNA structure.

In one example, QPCR is performed using real-time PCR (RTPCR), where the amount of product is monitored during the amplification reaction, or by end-point measurements, in which the amount of a final product is determined. As is known to a skilled person, rtPCR is for instance performed by the use of a nucleic acid intercalator, such as for example ethidium bromide or SYBR® Green I dye, which interacts which all generated double stranded products resulting in an increase in fluorescence during amplification, or for instance by the use of labeled probes that react specifically with the generated double stranded product of the gene of interest. Alternative detection methods that can be used are provided by amongst other things dendrimer signal amplification, hybridization signal amplification, and molecular beacons.

In one embodiment, reverse transcription on total RNA is carried out using the High Capacity cDNA Archive Kit (Applied Biosystems (ABI), Foster City, CA) following the manufacturer's suggested protocol (briefly, using 2 micrograms of total RNA in 50 microliters water, mixing with 50 uL of 2XRT mix containing Reverse Transcription Buffer, deoxynucleotide triphosphate solution, random primers, and Multiscribe Reverse Transcriptase). RT reaction conditions are well known. In one example, the RT reaction is performed using the following thermal cycler conditions: 10 mins, 25° C.; 120 min., 37° C. {see Kidd M, et al., “The role of genetic markers—NAP 1 LI, MAGE-D2, and MTA1—in defining small-intestinal carcinoid neoplasia,” Ann Surg Oncol 2006; 13 (2): 253-62).

For measurement of individual transcript levels, in one embodiment, Assays-on-Demand™ products are used with the ABI 7900 Sequence Detection System according to the manufacturer's suggestions (see Kidd M, Eick G, Shapiro M D, et al. Microsatellite instability and gene mutations in transforming growth factor-beta type II receptor are absent in small bowel carcinoid tumors. Cancer 2005; 103 (2): 229-36). In one example, cycling is performed under standard conditions, using the TaqMan® Universal PCR Master Mix Protocol, by mixing cDNA in 7.2 uL water, 0.8 uL 20·ASSAYS-ON-DEMAND™ (gene expression products) primer and probe mix and 8 uL of 2×TaqMan Universal Master mix, in a 384-well optical reaction plate, under the following conditions: 50° C., 2 min.; 95° C.; 10 min.; 50 cycles at 95° C. for 15 min., 60° for 1 min (see Kidd M, et al, “The role of genetic markers—NAP 1 LI, MAGE-D2, and MTA1—in defining small-intestinal carcinoid neoplasia,” Ann Surg Oncol 2006; 13 (2): 253-62).

Typically, results from real-time PCR are normalized, using internal standards and/or by comparison to expression levels for housekeeping genes. For example, in one embodiment, Raw AC_{T} (delta C_{T}=change in cycle time as a function of amplification) data from QPCR as described above is normalized using well-known methods, such as geNorm {see Vandesompele J, De Preter K, Pattyn F, et al. Accurate normalization of real-time quantitative RT-PCR data by geometric averaging of multiple internal control genes. Genome Biol 2002; 3 (7): RESEARCH0034). Normalization by house-keeping gene expression levels is also well-known. See Kidd M, et al., “GeneChip, geNorm, and gastrointestinal tumors: novel reference genes for real-time PCR,” Physiol Genomics 2007; 30 (3): 363-70.

Microarray analysis involves the use of selected nucleic acid molecules that are immobilized on a surface. These nucleic acid molecules, termed probes, are able to hybridize to nucleic acid expression products. In a preferred embodiment the probes are exposed to labeled sample nucleic acid, hybridized, washed and the (relative) amount of nucleic acid expression products in the sample that are complementary to a probe is determined. Microarray analysis allows simultaneous determination of nucleic acid expression levels of a large number of genes. In a method according to the invention it is preferred that at least 5 genes according to the invention are measured simultaneously.

Background correction can be performed for instance according to the “offset” method that avoids negative intensity values after background subtraction. Furthermore, normalization can be performed in order to make the two channels on each single array comparable for instance using global loess normalization, and scale normalization which ensures that the log-ratios are scaled to have the same median-absolute-deviation (MAD) across arrays.

Protein levels may, for example, be measured using antibody-based binding assays. Enzyme labeled, radioactively labeled or fluorescently labeled antibodies may be used for detection of protein. Exemplary assays include enzyme-linked immunosorbent assays (ELISA), radio-immuno assays (RIA), Western Blot assays and immunohistochemical staining assays. Alternatively, in order to determine the expression level of multiple proteins simultaneously protein arrays such as antibody-arrays are used.

Typically, the biomarkers and housekeeping markers are detected in a biological sample, such as a tissue or fluid sample, such as a blood, such as whole blood, plasma, serum, stool, urine, saliva, tears, serum or semen sample, or a sample prepared from such a tissue or fluid, such as a cell preparation, including of cells from blood, saliva, or tissue, such as intestinal mucosa, tumor tissue, and tissues containing and/or suspected of containing GEP-NEN metastases or shed tumor cells, such as liver, bone, and blood. In one embodiment, a specific cell preparation is obtained by fluorescence-activated cell sorting (FACS) of cell suspensions or fluid from tissue or fluid, such as mucosa, e.g., intestinal mucosa, blood or buffy coat samples.

In some embodiments, the sample is taken from a GEP-NEN patient, a patient suspected of having GEP-NEN, a patient having and/or suspected of having cancer generally, a patient exhibiting one or more GEP-NEN symptoms or syndromes or determined to be at-risk for GEP-NEN, or a GEP-NEN patient undergoing treatment or having completed treatment, including patients whose disease is and/or is thought to be in remission.

In other embodiments, the sample is taken from a human without GEP-NEN disease, such as a healthy individual or an individual with a different type of cancer, such as an adenocarcinoma, for example, a gastrointestinal adenocarcinoma or one of the breast, prostate, or pancreas, or a gastric or hepatic cancer, such as esophageal, pancreatic, gallbladder, colon, or rectal cancer.

In some embodiments, the sample is taken from the GEP-NEN tumor or metastasis. In other embodiments, the sample is taken from the GEP-NEN patient, but from a tissue or fluid not expected to contain GEP-NEN or GEP-NEN cells; such samples may be used as reference or normal samples. Alternatively, the normal or reference sample may be a tissue or fluid or other biological sample from a patient without GEP-NEN disease, such as a corresponding tissue, fluid or other sample, such as a normal blood sample, a normal small intestinal (SI) mucosa sample, a normal enterochromaffin (EC) cell preparation.

In some embodiments, the sample is a whole blood sample. As neuroendocrine tumors metastasize, they typically shed cells into the blood. Accordingly, detection of the panels of GEP-NEN biomarkers provided herein in plasma and blood samples may be used for identification of GEP-NENs at an early time point and for predicting the presence of tumor metastases, e.g., even if anatomic localization studies are negative. Accordingly, the provided agents and methods are useful for early diagnosis.

Thus, in some embodiments, the methods can identify a GEP-NEN molecular signature or expression profile in 1 mL or about 1 mL of whole blood. In some aspects, the molecular signature or expression profile is stable for up to four hours (for example, when samples are refrigerated 4-8° C. following phlebotomy) prior to freezing. In one aspect, the approach able to diagnose, prognosticate or predict a given GEP-NEN-associated outcome using a sample obtained from tumor tissue is also able to make the same diagnosis, prognosis, or prediction using a blood sample.

A number of existing detection and diagnostic methodologies require 7 to 10 days to produce a possible positive result, and can be costly. Thus, in one aspect, the provided methods and compositions are useful in improving simplicity and reducing costs associated with GEP-NEN diagnosis, and make early-stage diagnosis feasible.

Thus in one example, the biomarkers are detected in circulation, for example by detection in a blood sample, such as a serum, plasma, cells, e.g., peripheral blood mononuclear cells (PBMCs), obtained from buffy coat, or whole blood sample.

Tumor-specific transcripts have been detected in whole blood in some cancers. See Sieuwerts A M, et al., “Molecular characterization of circulating tumor cells in large quantities of contaminating leukocytes by a multiplex real-time PCR,” Breast Cancer Res Treat 2009; 118 (3): 455-68 and Mimori K, et al, “A large-scale study of MT1-MMP as a marker for isolated tumor cells in peripheral blood and bone marrow in gastric cancer cases,” Ann Surg Oncol 2008; 15 (10): 2934-42.

The CELLSEARCH® CTC Test (circulating tumor cell kits) (described by Kahan L., “Medical devices; immunology and microbiology devices, classification of the immunomagnetic circulating cancer cell selection and enumeration system. Final rule,” Fed Regist 2004; 69:26036-8) uses magnetic beads coated with EpCAM-specific antibodies that detects epithelial cells (CK-Aug. 18, 2019) and leukocytes (CD45), as described by Sieuwerts A M, Kraan J, Bolt-de Vries J, et al., “Molecular characterization of circulating tumor cells in large quantities of contaminating leukocytes by a multiplex real-time PCR,” Breast Cancer Res Treat 2009; 118 (3): 455-68. This method has been used to detect circulating tumor cells (CTCs), and monitoring disease progression and therapy efficacy in metastatic prostate (Danila D C, Heller G, Gignac G A, et al. Circulating tumor cell number and prognosis in progressive castration-resistant prostate cancer. Clin Cancer Res 2007; 13 (23): 7053-8), colorectal (Cohen S J, Alpaugh R K, Gross S, et al.).

Isolation and characterization of circulating tumor cells in patients with metastatic colorectal cancer. Clin Colorectal Cancer 2006; 6 (2): 125-32. and breast (Cristofanilli M, Budd G T, Ellis M J, et al., Circulating tumor cells, disease progression, and survival in metastatic breast cancer. N Engl J Med 2004; 351 (8): 781-91).

This and other existing approaches have not been entirely satisfactory for detection of GEP-NEN cells, which can exhibit variable expression and/or not express cytokeratin (See Van Eeden S, et al, Classification of low-grade neuroendocrine tumors of midgut and unknown origin, “Hum Pathol 2002; 33 (11): 1126-32; Cai Y C, et al., “Cytokeratin 7 and 20 and thyroid transcription factor 1 can help distinguish pulmonary from gastrointestinal carcinoid and pancreatic endocrine tumors,” Hum Pathol 2001; 32 (10): 1087-93, and studies described herein, detecting EpCAM transcript expression in two of twenty-nine GEP-NEN samples).

Factors to consider in the available detection methods for circulating tumor cells are relatively low numbers of the cells in peripheral blood, typically about 1 per 106 peripheral blood mononuclear cells (PBMCs) (see Ross A A, et al. “Detection and viability of tumor cells in peripheral blood stem cell collections from breast cancer patients using immunocytochemical and clonogenic assay techniques,” Blood 1993; 82 (9): 2605-10), and the potential for leukocyte contamination. See Sieuwerts A M, et al. “Molecular characterization of circulating tumor cells in large quantities of contaminating leukocytes by a multiplex real-time PCR,” Breast Cancer Res Treat 2009; 118 (3): 455-68; Mimori K, et al) and technical complexity of available approaches. These factors can render available methods not entirely satisfactory for use in the clinical laboratory.

In some embodiments, Neuroendocrine cells are FACS-sorted to heterogeneity, using known methods, following acridine orange (AO) staining and uptake, as described Kidd M, et al., “Isolation, Purification and Functional Characterization of the Mastomys EC cell,” Am J Physiol 2006; 291: G778-91; Modlin EVI, et al., “The functional characterization of normal and neoplastic human enterochromaffin cells,” Clin Endocrinol Metab 2006; 91 (6): 2340-8.

In some embodiments, the provided detection methods are used to detect, isolate, or enrich for the GEP-NEN cells and/or biomarkers in two to three mL of blood or less. The methods are performed using standard laboratory apparatuses and thus are easily performed in the clinical laboratory setting. In one example, a readout is obtained within 12 hours, at an average cost of approximately 20-30 per sample.

The present invention provides diagnostic, prognostic, and predictive uses for the agents and detection methods provided herein, such as for the diagnosis, prognosis, and prediction of GEP-NEN, associated outcomes, and treatment responsiveness. For example, available GEP-NEN classification methods are limited, in part due to incorrect classifications and that individual lesions or tumors can evolve into different GEP-NEN sub-types or patterns, and/or contain more than one GEP-NEN sub-type. Known classification frameworks are limited, for example, in the ability to predict response to treatment or discriminate accurately between tumors with similar histopathologic features that may vary substantially in clinical course and treatment response, and to predict treatment responsiveness.

There is therefore a need for molecular or gene-based classification schemes. The provided methods and systems, including GEP-NEN-specific predictive gene-based models, address these issues, and may be used in identifying and analyzing molecular parameters that are predictive of biologic behavior and prediction based on such parameters.

Among the provided diagnostic, prognostic, and predictive methods are those which employ statistical analysis and biomathematical algorithms and predictive models to analyze the detected information about expression of GEP-NEN biomarkers and other markers such as housekeeping genes. In some embodiments, expression levels, detected binding or other information is normalized and assessed against reference value(s), such as expression levels in normal samples or standards. Provided embodiments include methods and systems for classification and prediction of GEP-NENs using the detected and measured information about the expression of the GEP-NEN biomarkers, for example, in classification, staging, prognosis, treatment design, evaluation of treatment options, and prediction of GEP-NEN disease outcomes, e.g., predicting development of metastases.

In some embodiments, the methods are used to establish GEP-NEN diagnosis, such as diagnosis or detection of early-stage disease or metastasis, define or predict the extent of disease, identify early spread or metastasis, predict outcome or prognosis, predict progression, classify disease activity, monitor treatment responsiveness, detect or monitor for recurrence, and to facilitate early therapeutic intervention. For example, among the provided methods and algorithms are those for use in classification, staging, prognosis, treatment design, evaluation of treatment options, and prediction of GEP-NEN disease outcomes, e.g., predicting development of metastases.

In one embodiment, the methods, algorithms and models are useful for diagnostic surveillance, such as routine surveillance and patient follow-up. In some embodiments, the methods, algorithms and models provide for early diagnosis; in one aspect, the methods are capable of detection of low-volume tumors, and detection of circulating tumor cells, including at early stages of disease, such as detection of as few as at or about 3 circulating GEP-NEN cells per milliliter of blood. In some embodiments, early evidence of disease allows early therapeutic intervention, at a time when therapies are more effective, which can improve survival rates and disease outcomes.

For example, in one embodiment, the methods useful for early detection of the recurrence and/or metastasis of GEP-NEN, such as after treatment for example following surgical or chemical intervention. In some aspect, the methods are performed weekly or monthly following therapeutic intervention, for example, on human blood samples. In some aspects, the methods are capable of detecting micrometastases that are too small to be detected by conventional means, such as by imaging methods. For example, in one aspect the methods are capable of detecting metastases less than one centimeter (cm), such as at or about 1 cm, 0.9 cm, 0.8 cm, 0.7 cm, 0.6 cm, 0.5 cm, 0.4 cm, 0.3 cm, 0.2 cm, or 0.1 cm metastases, such as in the liver.

For example, among the provided methods and systems are those that determine the presence or absence (or both) of a GEP-NEN in a subject or sample with a correct call rate of between 56 and 92%, such as at least or at least about a 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% correct call rate. In some cases, the methods are useful for diagnosis with a specificity or sensitivity of at least or at least about 70%, 7%5, 80%, 81%, 82%, 83%, 84%, 85%, 86%, 87%, 88%, 89%, 90%, 91%, 92%, 93%, 94%, 95%, 96%, 97%, 98%, 99%, or 100%.

In other aspects, the methods are capable of detecting the recurrence, metastasis, or spread of GEP-NEN following treatment or during initial disease progression at an earlier stage as compared with other diagnostic methods, such as imaging and detection of available biomarkers. In some aspects, the detected expression levels and/or expression signature of the biomarkers correlate significantly with the progression of disease, disease severity or aggressiveness, lack of responsiveness of treatment, reduction in treatment efficacy, GEP-NEN-associated events, risk, prognosis, type or class of GEP-NEN or disease stage.

Among the provided embodiments are methods that use the provided biomarkers and detection thereof in treatment development, strategy, and monitoring, including evaluation of response to treatment and patient-specific or individualized treatment strategies that take into consideration the likely natural history of the tumor and general health of the patient.

GEP-NEN management strategies include surgery—for cure (rarely achieved) or cytoreduction-radiological intervention—for example, by chemoembolization or radiofrequency ablation-chemotherapy, cryoablation, and treatment with somatostatin and somatostatin analogues (such as SANDOSTATIN® LAR (Octreotide acetate injection)) to control symptoms caused by released peptides and neuroamines. Biological agents, including interferon, and hormone therapy, and somatostatin-tagged radionucleotides are under investigation.

In one example, Cryoablation liberates GEP-NEN tissue for entry into the blood, which in turn induces symptoms, as described by Mazzaglia P J, et ah, “Laparoscopic radiofrequency ablation of neuroendocrine liver metastases: a 10-year experience evaluating predictors of survival,” Surgery 2007,142 (1): 10-9. Chemo therapeutic agents, e.g., systemic cytotoxic chemo therapeutic agents, include etoposide, cisplatin, 5-fluorouracil, streptozotocin, doxorubicin; vascular endothelial growth factor inhibitors, receptor tyrosine kinase inhibitors (e.g., Sunitinib, Sorafenib, and Vatalanib), and mammalian target of rapamycin (mTOR) inhibitors (e.g., Temsirolimus and Everolimus), and combinations thereof, for example to treat disseminated and/or poorly differentiated/aggressive disease. Other treatment approaches are well known.

In some embodiments, the detection and diagnostic methods are used in conjunction with treatment, for example, by performing the methods weekly or monthly before and/or after treatment. In some aspects, the expression levels and profiles correlate with the progression of disease, ineffectiveness or effectiveness of treatment, and/or the recurrence or lack thereof of disease. In some aspects, the expression information indicates that a different treatment strategy is preferable. Thus, provided herein are therapeutic methods, in which the GEP-NEN biomarker detection methods are performed prior to treatment, and then used to monitor therapeutic effects.

At various points in time after initiating or resuming treatment, significant changes in expression levels or expression profiles of the biomarkers (e.g., as compared to expression or expression profiles before treatment, or at some other point after treatment, and/or in a normal or reference sample) indicates that a therapeutic strategy is or is not successful, that disease is recurring, or that a different therapeutic approach should be used. In some embodiments, the therapeutic strategy is changed following performing of the detection methods, such as by adding a different therapeutic intervention, either in addition to or in place of the current approach, by increasing or decreasing the aggressiveness or frequency of the current approach, or stopping or reinstituting the treatment regimen.

In another aspect, the detected expression levels or expression profile of the biomarkers identifies the GEP-NEN disease for the first time or provides the first definitive diagnosis or classification of GEP-NEN disease. In some aspects of this embodiment, a treatment approach is designed based upon the expression levels or expression profiles, and/or the determined classification. The methods include iterative approaches, whereby the biomarker detection methods are followed by initiation or shift in therapeutic intervention, followed by continued periodic monitoring, reevaluation, and change, cessation, or addition of a new therapeutic approach, optionally with continued monitoring.

In some aspects, the methods and systems determine whether or not the assayed subject is responsive to treatment, such as a subject who is clinically categorized as in complete remission or exhibiting stable disease. In some aspects, the methods and systems determine whether or not the subject is untreated (or treatment-I, i.e., has not received treatment) or is non-responsive (i.e., clinically categorized as “progressive.” For example, methods are provided for distinguishing treatment-responsive and non-responsive patients, and for distinguishing patients with stable disease or those in complete remission, and those with progressive disease. In various aspects, the methods and systems make such calls with at least at or about 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% correct call rate (i.e., accuracy), specificity, or sensitivity.

In some aspects, the sensitivity or correct call rate for the diagnostic or predictive or prognostic outcome is greater than, e.g., significantly greater than, that obtained using a known diagnosis or prognostic method, such as detection and measurement of circulating CgA or other single protein.

Typically, the diagnostic, prognostic, and predictive methods, often in an initial step, select a subset of biomarkers based on their ability to build a classifier that may accurately predict and classify GEP-NEN and/or different stages of GEP-NEN.

Any of a number of well-known methods for evaluating differences in gene expression may be used to select the subset of biomarkers. For example, an accurate classifier may be based on topographic, pattern-recognition based protocols e.g., support vector machines (SVM) (Noble W S. What is a support vector machine? Nat Biotechnol. 2006; 24 (12): 1565-7). Machine-learning based techniques are typically desirable for developing sophisticated, automatic, and/or objective algorithms for analyzing high-dimensional and multimodal biomedical data. In some examples, SVM—a variant of the supervised learning algorithm—is used in connection with the provided methods and systems. SVMs have been used to predict the grading of astrocytomas with a >90 accuracy, and prostatic carcinomas with an accuracy of 74-80% (Glotsos D, Tohka J, Ravazoula P, Cavouras D, Nikiforidis G. Automated diagnosis of brain tumours astrocytomas using probabilistic neural network clustering and support vector machines. Int J Neural Syst 2005; 15 (1-2): 1-11; Glotsos D, Tohka J, Ravazoula P, Cavouras D, Nikiforidis G. Automated diagnosis of brain tumours astrocytomas using probabilistic neural network clustering and support vector machines. Int J Neural Syst 2005; 15 (1-2): 1-11).

Other algorithms for building an accurate classifier include linear discriminant analysis (LDA), naive Bayes (NB), and K-nearest neighbor (KNN) protocols. Such approaches are useful for identifying individual or multi-variable alterations in neoplastic conditions (Drozdov I, Tsoka S, Ouzounis C A, Shah A M. Genome-wide expression patterns in physiological cardiac hypertrophy. BMC Genomics. 2010; 11:55; Freeman T C, Goldovsky L, Brosch M, et al. Construction, visualization, and clustering of transcription networks from microarray expression data. PLoS Comput Biol 2007; 3 (10): 2032-42; Zampetaki A, Kiechl S, Drozdov I, et al. Plasma microRNA profiling reveals loss of endothelial miR-126 and other microRNAs in type 2 diabetes. Circ Res. 2010; 107 (6): 810-7. Epub 2010 Jul. 22; Dhawan M, Selvaraja S, Duan Z H. Application of committee kNN classifiers for gene expression profile classification. Int J Bioinform Res Appl. 2010; 6 (4): 344-52; Kawarazaki S, Taniguchi K, Shirahata M, et al. Conversion of a molecular classifier obtained by gene expression profiling into a classifier based on real-time PCR: a prognosis predictor for gliomas. BMC Med Genomics. 2010; 3:52; Vandebriel R J, Van Loveren H, Meredith C. Altered cytokine (receptor) mRNA expression as a tool in immunotoxicology. Toxicology. 1998; 130 (1): 43-67; Urgard E, Vooder T, Vosa U, et al. Metagenes associated with survival in non-small cell lung cancer. Cancer Inform. 2011; 10:175-83. Epub 2011 Jun. 2; Pimentel M, Amichai M, Chua K, Braham L. Validating a New Genomic Test for Irritable Bowel Syndrome Gastroenterology 2011; 140 (Suppl 1): S-798; Lawlor G. Rosenberg L, Ahmed A, et al. Increased Peripheral Blood GATA-3 Expression in Asymptomatic Patients With Active Ulcerative Colitis at Colonoscopy. Gastroenterology 2011; 140 (Suppl 1)).

In some embodiments, an accurate classifier for GEP-NEN and/or different stages of GEP-NEN is based on a combination of the SVM, LDA, NB, and KNN protocols. This is termed the Multi-Analyte-Algorithm Risk Classifier for NETs (MAARC-NET).

Methods using the predictive algorithms and models use statistical analysis and data compression methods, such as those well known in the art. For example, expression data may be transformed, e.g., In-transformed, and imported into a statistical analysis program, such as PARTEK® GENOMICS SUITE® (genomic data analysis software) or similar program, for example. Data are compressed and analyzed for comparison.

Whether differences in expression level score or values are deemed significant may be determined by well-known statistical approaches, and typically is done by designating a threshold for a particular statistical parameter, such as a threshold p-value (e.g., p<0.05), threshold S-value (e.g., +0.4, with S<−0.4 or S>0.4), or other value, at which differences are deemed significant, for example, where expression of a biomarker is considered significantly down- or up-regulated, respectively, among two different samples, for example, representing two different GEP-NEN sub-types, tumors, stages, localizations, aggressiveness, or other aspect of GEP-NEN or normal or reference sample.

In one aspect, the algorithms, predictive models, and methods are based on biomarkers expressed from genes associated with regulatory gene clusters (i.e., SSTRome, Proliferome. Signalome. Metabolome, Secretome, Secretome, Plurome, EpiGenome, and Apoptome) underlying various GEP-NEN subtypes.

In one aspect, the methods apply the mathematical formulations, algorithms or models identify specific cutoff points, for example, pre-determined expression level scores, which distinguish between normal and GEP-NEN samples, between GEP-NEN and other cancers, and between various sub-types, stages, and other aspects of disease or disease outcome. In another aspect, the methods are used for prediction, classification, prognosis, and treatment monitoring and design. In one aspect, the predictive embodiments are useful for identifying molecular parameters predictive of biologic behavior, and prediction of various GEP-NEN-associated outcomes using the parameters. In one aspect of these embodiments, machine learning approaches are used, e.g., to develop sophisticated, automatic and objective algorithms for the analysis of high-dimensional and multimodal biomedical data.

A “ROC curve” as used herein refers to a plot of the true positive rate (sensitivity) against the false positive rate (specificity) for a binary classifier system as its discrimination threshold is varied. A ROC curve can be represented equivalently by plotting the fraction of true positives out of the positives (TPR=true positive rate) versus the fraction of false positives out of the negatives (FPR=false positive rate). Each point on the ROC curve represents a sensitivity/specificity pair corresponding to a particular decision threshold.

AUC represents the area under the ROC curve. The AUC is an overall indication of the diagnostic accuracy of 1) a subset or panel of GEP-NEN biomarkers and 2) a ROC curve. AUC is determined by the “trapezoidal rule.” For a given curve, the data points are connected by straight line segments, perpendiculars are erected from the abscissa to each data point, and the sum of the areas of the triangles and trapezoids so constructed is computed. In certain embodiments of the methods provided herein, a subset or panel of GEP-NEN has an AUC in the range of about 0.75 to 1.0. In certain of these embodiments, the AUC is in the range of about 0.50 to 0.85, 0.85 to 0.9, 0.9 to 0.95, or 0.95 to 1.0.

For the comparison of expression level scores or other values, and to identify expression profiles (expression signatures) or regulatory signatures based on GEP-NEN biomarker expression, data are compressed. Compression typically is by Principal Component Analysis (PCA) or similar technique for describing and visualizing the structure of high-dimensional data. PCA allows the visualization and comparison of GEP-NEN biomarker expression and determining and comparing expression profiles (expression signatures, expression patterns) among different samples, such as between normal or reference and test samples and among different tumor types.

In some embodiments, expression level data are acquired, e.g., by real-time PCR, and reduced or compressed, for example, to principal components.

PCA is used to reduce dimensionality of the data (e.g., measured expression values) into uncorrelated principal components (PCs) that explain or represent a majority of the variance in the data, such as about 50%, 60%, 70%, 75%, 80%, 85%, 90%, 95%, or 99% of the variance.

In one example, the PCA is 3-component PCA, in which three PCs are used that collectively represent most of the variance, for example, about 75%, 80%, 85%, 90%, or more variance in the data (Jolliffe I T, “Principle Component Analysis,” Springer, 1986).

PCA mapping, e.g., 3-component PCA mapping is used to map data to a three dimensional space for visualization, such as by assigning first (1^{st}), second (2^{nd}) and third (3^{rd}) PC's to the X-, Y-, and Z-axes, respectively.

PCA may be used to determine expression profiles for the biomarkers in various samples. For example, reduced expression data for individual sample types (e.g., each tumor type, sub-type or grade, or normal sample type) are localized in a PCA coordinate system and localized data used to determine individual transcript expression profiles or signatures.

In one aspect, the expression profile is determined for each sample by plotting or defining a centroid (center of mass; average expression), corresponding to or representing the sample's individual transcript expression profile (regulatory signature), as given by the principal component vector, as determined by PCA for the panel of biomarkers.

Generally, two centroids or points of localization separated by a relatively large distance in this coordinate system represent two relatively distinct transcript expression profiles. Likewise, relatively close centroids represent relatively similar profiles. In this representation, the distance between centroids is inversely equivalent to the similarity measure (greater distance=less similarity) for the different samples, such that large distances or separation between centroids indicates samples having distinct transcript expression signatures. Proximity of centroids indicates similarity between samples. For example, the relative distance between centroids for different GEP-NEN tumor samples represents the relative similarity of their regulatory signatures or transcript expression profiles.

In one aspect, the statistical and comparative analysis includes determining the inverse correlation between expression levels or values for two biomarkers. In one example, this correlation and the cosine of the angle between individual expression vectors (greater angle=less similarity), is used to identify related gene expression clusters (Gabriel K R, “The biplot graphic display of matrices with application to principal component analysis,” Biometrika 1971; 58 (3): 453).

In some embodiments, there is a linear correlation between expression levels of two or more biomarkers, and/or the presence or absence of GEP-NEN, sub-type, stage, or other outcome. In one aspect, there is an expression-dependent correlation between the provided GEP-NEN biomarkers and characteristics of the biological samples, such as between biomarkers (and expression levels thereof) and various GEP-NEN sub-types (e.g., benign versus active disease).

Pearson's Correlation (PC) coefficients (R2) may be used to assess linear relationships (correlations) between pairs of values, such as between expression levels of a biomarker for different biological samples (e.g., tumor sub-types) and between pairs of biomarkers. This analysis may be used to linearly separate distribution in expression patterns, by calculating PC coefficients for individual pairs of the biomarkers (plotted on x- and y-axes of individual Similarity Matrices). Thresholds may be set for varying degrees of linear correlation, such as a threshold for highly linear correlation of (R>0.50, or 0.40). Linear classifiers can be applied to the datasets. In one example, the correlation coefficient is 1.0.

In one embodiment, regulatory clusters are determined by constructing networks of correlations using statistical analyses, for example, to identify regulatory clusters composed of subsets of the panel of biomarkers. In one example, PC correlation coefficients are determined and used to construct such networks of correlations. In one example, the networks are identified by drawing edges between transcript pairs having R above the pre-defined threshold. Degree of correlation can provide information on reproducibility and robustness.

Also provided herein are objective algorithms, predictive models, and topographic analytical methods, and methods using the same, to analyze high-dimensional and multimodal biomedical data, such as the data obtained using the provided methods for detecting expression of the GEP-NEN biomarker panels. As discussed above, the objective algorithms, models, and analytical methods include mathematical analyses based on topographic, pattern-recognition based protocols e.g., support vector machines (SVM) (Noble W S. What is a support vector machine? Nat Biotechnol. 2006; 24 (12): 1565-7), linear discriminant analysis (LDA), naive Bayes (NB), and K-nearest neighbor (KNN) protocols, as well as other supervised learning algorithms and models, such as Decision Tree, Perceptron, and regularized discriminant analysis (RDA), and similar models and algorithms well-known in the art (Gallant S I, “Perceptron-based learning algorithms,” Perceptron-based learning algorithms 1990; 1 (2): 179-91).

In some embodiments, Feature Selection (FS) is applied to remove the most redundant features from a dataset, such as a GEP-NEN biomarker expression dataset, and generate a relevant subset of GEP-NEN biomarkers. FS enhances the generalization capability, accelerates the learning process, and improves model interpretability. In one aspect, FS is employed using a “greedy forward” selection approach, selecting the most relevant subset of features for the robust learning models. (Peng H, Long F, Ding C, “Feature selection based on mutual information: criteria of max-dependency, max-relevance, and min-redundancy,” IEEE Transactions on Pattern Analysis and Machine Intelligence, 2005; 27 (8): 1226-38).

In some embodiments, Support Vector Machines (SVM) algorithms are used for classification of data by increasing the margin between the n data sets (Cristianini N, Shawe-Taylor J. An Introduction to Support Vector Machines and other kernel-based learning methods. Cambridge: Cambridge University Press, 2000).

In some embodiments, the predictive models include Decision Tree, which maps observations about an item to a conclusion about its target value (Zhang H, Singer B. “Recursive Partitioning in the Health Sciences,” (Statistics for Biology and Health): Springer, 1999.). The leaves of the tree represent classifications and branches represent conjunctions of features that devolve into the individual classifications. It has been used effectively (70-90%) to predict prognosis of metastatic breast cancer (Yu L et al “TGF-beta receptor-activated p38 MAP kinase mediates Smad-independent TGF-beta responses,” Embo J 2002; 21 (14): 3749-59), as well as colon cancer (Zhang H et al “Recursive partitioning for tumor classification with gene expression microarray data.,” Proc Natl Acad Sci USA 2001; 98 (12): 6730-5), to predict the grading of astrocytomas (Glotsos D et al “Automated diagnosis of brain tumours astrocytomas using probabilistic neural network clustering and support vector machines,” Int J Neural Syst 2005; 15 (1-2): 1-11) with a >90% accuracy, and prostatic carcinomas with an accuracy of 74-80% (Mattfeldt T et al. “Classification of prostatic carcinoma with artificial neural networks using comparative genomic hybridization and quantitative stereological data,” Pathol Res Pract 2003; 199 (12): 773-84). The efficiency of this technique has been measured by 10-fold cross-validation (Pirooznia M et al “A comparative study of different machine learning methods on microarray gene expression data,” BMC Genomics 2008; 9 Suppl 1: S13).

The predictive models and algorithms further include Perceptron, a linear classifier that forms a feed forward neural network and maps an input variable to a binary classifier (Gallant S I. “Perceptron-based learning algorithms,” Perceptron-based learning algorithms 1990; 1 (2): 179-91). It has been used to predict malignancy of breast cancer (Markey M K et al. “Perceptron error surface analysis: a case study in breast cancer diagnosis,” Comput Biol Med 2002; 32 (2): 99-109). In this model, the learning rate is a constant that regulates the speed of learning. A lower learning rate improves the classification model, while increasing the time to process the variable (Markey M K et al. “Perceptron error surface analysis: a case study in breast cancer diagnosis,” Comput Biol Med 2002; 32 (2): 99-109). In one example, a learning rate of 0.05 is used. In one aspect, a Perceptron algorithm is used to distinguish between localized or primary tumors and corresponding metastatic tumors. In one aspect, three data scans are used to generate decision boundaries that explicitly separate data into classes.

The predictive models and algorithms further include Regularized Discriminant Analysis (RDA), which can be used as a flexible alternative to other data mining techniques, including Linear and Quadratic Discriminant Analysis (LDA, QDA) (Lilien R H, Farid H, Donald B R. “Probabilistic disease classification of expression-dependent proteomic data from mass spectrometry of human serum.,” J Comput Biol 2003; 10 (6): 925-46.; Cappellen D. Luong-Nguyen N H, Bongiovanni S, et al. “Transcriptional program of mouse osteoclast differentiation governed by the macrophage colony-stimulating factor and the ligand for the receptor activator of NFkappa B.” J Biol Chem 2002; 277 (24): 21971-82.). RDA's regularization parameters, γ and λ, are used to design an intermediate classifier between LDA and QDA. QDA is performed when γ=0 and λ\hat{ }O while LDA is performed when γ=0 and λ=1 (Picon A, Gold L I, Wang J, Cohen A, Friedman E. A subset of metastatic human colon cancers expresses elevated levels of transforming growth factor beta 1. Cancer Epidemiol. Biomarkers Prev. 1998; 7 (6): 497-504).

To reduce over-fitting, RDA parameters are selected to minimize cross-validation error while not being equal 0.0001, thus forcing RDA to produce a classifier between LDA, QDA, and L2 (Pima I, Aladjem M., “Regularized discriminant analysis for face recognition,” Pattern Recognition 2003; 37 (9): 1945-48). Finally, regularization itself has been used widely to overcome over-fitting in machine learning (Evgeniou T, Pontil M, Poggio T. “Regularization Networks and Support Vector Machines.,” Advances in Computational Math 2000; 13 (1): 1-50.; Ji S, Ye J. Kernel “Uncorrelated and Regularized Discriminant Analysis: A Theoretical and Computational Study.,” IEEE Transactions on Knowledge and Data Engineering 2000; 20 (10): 1311-21.).

In one example, regularization parameters are defined as γ=0.002 and λ=0. In one example, for each class pair, S-values are assigned to all transcripts which are then arranged by a decreasing S-value. RDA is performed, e.g., 21 times, such that the N^{th} iteration consists of top N scoring transcripts. Error estimation can be carried out by a 10-fold cross-validation of the RDA classifier. This can be done by partitioning the tissue data set into complementary subsets, performing the analysis on one subset (called the training set), and validating the analysis on the other subset (called the validation set or testing set).

In one example, misclassification error is averaged to reduce variability in the overall predictive assessment, which can provide a more accurate approach to error estimation compared to other approaches, including bootstrapping and leave-one-out cross-validation (Kohavi R. “A study of cross-validation and bootstrap for accuracy estimation and model selection.,” Proceedings of the Fourteenth International Joint Conference on Artificial Intelligence, 1995; 2 (12): 1137-43.).

In one example, selection for tissue classification is performed, for example, by computing the rank score(S) for each gene and for each class pair as:

$$S = \frac{\vert {μ}_{C 2} - {μ}_{C 1} \vert}{{σ}_{C 1} + {σ}_{C 2}}$$

where μ_{C1} and μ_{C2} represent means of first and second class respectively and σ_{C1} and σ_{C2} are inter-class standard deviations. A large S value is indicative of a substantial differential expression (“Fold Change”) and a low standard deviation (“transcript stability”) within each class. Genes may be sorted by a decreasing S-value and used as inputs for the regularized discriminant analysis algorithm (RDA).

The algorithms and models may be evaluated, validated and cross-validated, for example, to validate the predictive and classification abilities of the models, and to evaluate specificity and sensitivity. In one example, radial basis function is used as a kernel, and a 10-fold cross-validation used to measure the sensitivity of classification (Cristianini N, Shawe-Taylor J. “An Introduction to Support Vector Machines and other kernel-based learning methods.,” Cambridge: Cambridge University Press, 2000.). Various classification models and algorithms may be compared by the provided methods, for example, using training and cross-validation, as provided herein, to compare performance of the predictive models for predicting particular outcomes.

Embodiments of the provided methods, systems, and predictive models are reproducible, with high dynamic range, can detect small changes in data, and are performed using simple methods, at low cost, e.g., for implementation in a clinical laboratory.

Kits and other articles of manufacture are provided for use in the diagnostic, prognostic, predictive, and therapeutic applications described herein. In some embodiments, the kits include a carrier, package, or packaging, compartmentalized to receive one or more containers such as vials, tubes, plates, and wells, in which each of the containers includes one of the separate elements for use in the methods provided herein, and in some aspects further include a label or insert with instructions for use, such as the uses described herein. In one example, the individual containers include individual agents for detection of the GEP-NEN biomarkers as provided herein; in some examples, individual containers include agents for detection of housekeeping genes and/or normalization.

For example, the container(s) can comprise an agent, such as a probe or primer, which is or can be detectably labeled. Where the method utilizes nucleic acid hybridization for detection, the kit can also have containers containing nucleotide(s) for amplification of the target nucleic acid sequence. Kits can comprise a container comprising a reporter, such as a biotin-binding protein, such as avidin or streptavidin, bound to a reporter molecule, such as an enzymatic, fluorescent, or radioisotope label; such a reporter can be used with, e.g., a nucleic acid or antibody.

The kits will typically comprise the container(s) described above and one or more other containers associated therewith that comprise materials desirable from a commercial and user standpoint, including buffers, diluents, filters, needles, syringes; carrier, package, container, vial and/or tube labels listing contents and/or instructions for use, and package inserts with instructions for use.

A label can be present on or with the container to indicate that the composition is used for a specific therapeutic or non-therapeutic application, such as a prognostic, prophylactic, diagnostic or laboratory application, and can also indicate directions for either in vivo or in vitro use, such as those described herein. Directions and or other information can also be included on an insert(s) or label(s) which is included with or on the kit. The label can be on or associated with the container. A label a can be on a container when letters, numbers or other characters forming the label are molded or etched into the container itself; a label can be associated with a container when it is present within a receptacle or carrier that also holds the container, e.g., as a package insert. The label can indicate that the composition is used for diagnosing, treating, prophylaxing or prognosing a condition, such as GEP-NEN.

In another embodiment, an article(s) of manufacture containing compositions, such as amino acid sequence(s), small molecule(s), nucleic acid sequence(s), and/or antibody(s), e.g., materials useful for the diagnosis, prognosis, or therapy of GEP-NEN is provided. The article of manufacture typically comprises at least one container and at least one label. Suitable containers include, for example, bottles, vials, syringes, and test tubes. The containers can be formed from a variety of materials such as glass, metal or plastic. The container can hold amino acid sequence(s), small molecule(s), nucleic acid sequence(s), cell population(s) and/or antibody(s). In one embodiment, the container holds a polynucleotide for use in examining the mRNA expression profile of a cell, together with reagents used for this purpose. In another embodiment a container comprises an antibody, binding fragment thereof or specific binding protein for use in evaluating protein expression of GEP-NEN biomarkers in biological samples, e.g., blood or cells and tissues, or for relevant laboratory, prognostic, diagnostic, prophylactic and therapeutic purposes; indications and/or directions for such uses can be included on or with such container, as can reagents and other compositions or tools used for these purposes.

The article of manufacture can further comprise a second container comprising a pharmaceutically-acceptable buffer, such as phosphate-buffered saline, Ringer's solution and/or dextrose solution. It can further include other materials desirable from a commercial and user standpoint, including other buffers, diluents, filters, stirrers, needles, syringes, and/or package inserts with indications and/or instructions for use.

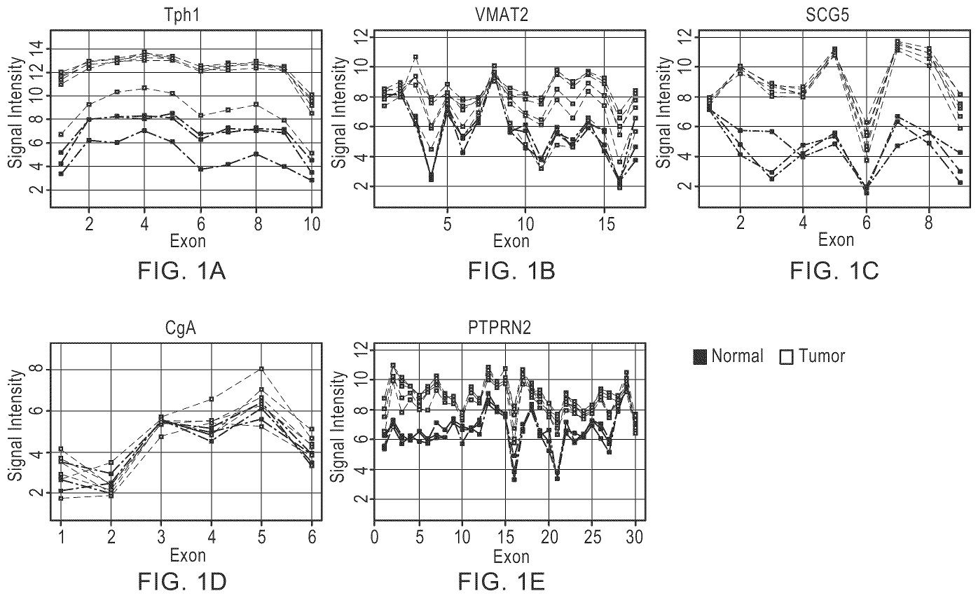

Differential Expression of NET Marker GENESIN Primary NETs—An exon-level screen of localized small intestinal NETs using Affymetrix Human Exon 1.0 ST arrays was performed to define alternative splicing events in neuroendocrine tumor tissue in comparison to a control (normal intestinal mucosa). Exon expression analysis identified 1287 differentially expressed genes between normal intestinal mucosa and NET tumor tissues. Five hundred and twenty nine genes were upregulated and 758 were downregulated. As an example, a subset of NET marker genes was focused on, in particular CgA, Tph1, VMAT2, SCG5, and PTPRN2. The RMA-normalized exon expression of the NET marker genes in this subset is shown in FIGS. 1A-1E in normal (green) and tumor (red) samples. Of these genes, Tph1 was the only gene where all exons were differentially expressed in tumor (FC>1.5, p<0.05), while CgA was the only gene where all exon expressions remained constant between tumor and normal samples.

Two of 17 differentially expressed exons were identified in VMAT2 and eight of 9 in SCG5. In PTPRN2 six of 30 exons were differentially expressed. These results demonstrate that specific primer/probe sets are required to maximize differences between neoplasia and normal gene expression.

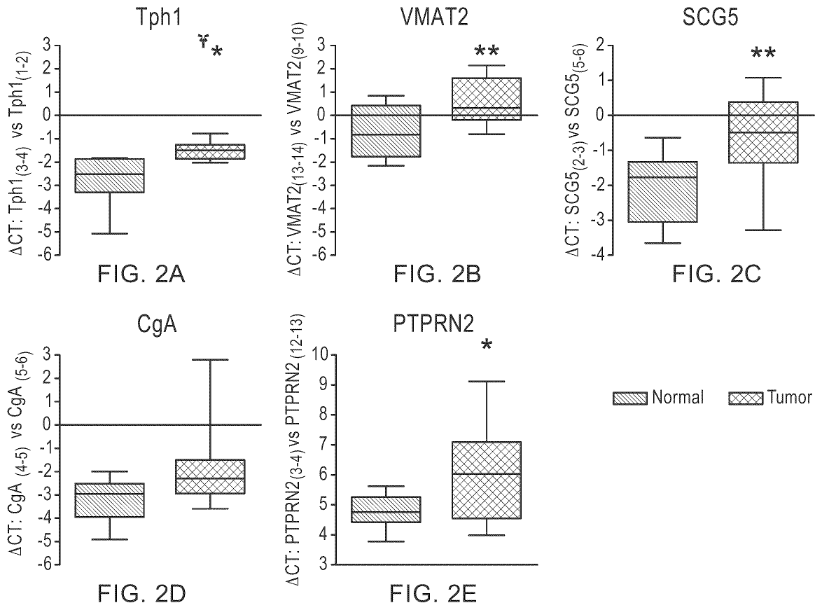

Validating Alternative Splicing in NET Marker Genes by RT-PCR—With reference to FIGS. 2A-2E, the findings of differential exon transcript levels was validated using reverse transcriptase polymerase chain reaction (RT-PCR). All marker gene exons, including Tph1₁₋₂, VMAT2₉₋₁₀, SCG5₂₋₃, and PTPRN2₁₂₋₁₃, were confirmed to be differentially expressed in tumor samples versus normal mucosa, with the exception of Cg₄₋₅.

Genomic and RT-PCR data from FIGS. 1A-1E and 2A-2E, respectively, identify that differential splicing occurs in NETs and that candidate biomarkers, e.g., VMAT2, require the use of specific primer/probe sets to effectively capture differences in expression of target transcripts.

To evaluate the relevance in blood, a microarray analysis of peripheral NET blood samples was performed. Up-regulated genes (n=1,397) included GO-Fat terms such as “RNA splicing”, “Vesicle-mediated transport”, and “Chromatin modification” which is consistent with known roles for these processes in NET pathobiology. Comparisons of the blood transcriptome with GEP-NET transcriptomes identified 236 up-regulated genes, 72 of which were examined for utility as biomarkers. A preliminary screen identified 51 genes as upregulated in tumor blood samples compared to controls. Forty-two genes (83%) were transcribed from multiple exons. A minimum of two primer/probe sets were tested for these genes in blood to define the most relevant combinations for target amplification. The housekeeping gene and 51 validated targets and exons of interest for primer/probe sets are described in TABLE 2. The amplicon positions identified for each GEN-NEN biomarker in Table 2 are the identified as underlined sequences in Table 1.

TABLE 2
Primer Details

| GEP-NEN | | NCBI | | | Amplicon | | |
|---|---|---|---|---|---|---|---|
| Biomarker | | Chromosome | UniGene | | Size | Exon | |
| Symbol | Name | location | ID | RefSeq | Length | Boundary | Position |
| ALG9 | asparagine-linked | Chr. 11- | Hs.503850 | NM_024740.2 | 68 | 4-5 | 541-600 |
| | glycosylation 9, | 111652919- | | | | | |
| | alpha-1,2-mannosyl | 111742305 | | | | | |
| | transferase homolog | | | | | | |
| AKAP8L | A kinase (PRKA) | Chr.19: | Hs.399800 | NM_014371.3 | 75 | 12-13 | 1596-1670 |
| | anchor protein 8-like | 15490859- | | | | | |
| | | 15529833 | | | | | |
| APLP2 | amyloid beta (A4) | Chr. 11- | Hs.370247 | NM_001142276.1 | 102 | 14-15 | 2029-2132 |
| | precursor-like | 129939716- | | | | | |
| | protein 2 | 130014706 | | | | | |
| ARAF1 | v-raf murine sarcoma | Chr. X- | Hs.446641 | NM_001654.4 | 74 | 10-11 | 1410-1475 |
| | 3611 viral oncogene | 47420578- | | | | | |
| | homolog | 47431320 | | | | | |
| ATP6V1H | ATPase, H + | Chr.8: | Hs.491737 | NM_015941.3 | 102 | 13-14 | 1631-1732 |
| | transporting, | 54628115- | | | | | |
| | lysosomal | 54755850 | | | | | |
| | 50/57 kDa, V1, | | | | | | |
| | Subunit H | | | | | | |
| BNIP3L | BCL2/adenovirus | Chr.8: | Hs.131226 | NM_004331.2 | 69 | 2-3 | 374-342 |
| | E1B 19 kDa | 26240523- | | | | | |
| | interacting | 26270644 | | | | | |
| | protein 3-like | | | | | | |
| BRAF | v-raf murine sarcoma | Chr. 7- | Hs.550061 | NM_004333.4 | 77 | 1-2 | 165-233 |
| | viral oncogene | 140433812- | | | | | |
| | homolog B1 | 140624564 | | | | | |
| C21ORF7 | chromosome 21 open | Chr.21: | Hs.222802 | NM_020152.3 | 76 | — | 611-686 |
| | reading frame 7 | 30452873- | | | | | |
| | | 30548204 | | | | | |
| CD59 | CD59 molecule, | Chr. 11- | Hs.278573 | NM_203331.2 | 70 | 3-4 | 193-264 |
| | complement | 33724556- | | | | | |
| | regulatory protein | 33758025 | | | | | |
| COMMD9 | COMM domain | Chr.11: | Hs.279836 | NM_001101653.1 | 85 | 2-3 | 191-275 |
| | containing 9 | 36293842- | | | | | |
| | | 36310999 | | | | | |
| CTGF | connective tissue | Chr. 6- | Hs.410037 | NM_001901.2 | 60 | 4-5 | 929-990 |
| | growth factor | 132269316- | | | | | |
| | | 132272518 | | | | | |
| ENPP4 | ectonucleotide | Chr.6: | Hs.643497 | NM_014936.4 | 82 | 3-4 | 1221-1303 |
| | pyrophos phatase/ | 46097701- | | | | | |
| | phosphodiesterase 4 | 46114436 | | | | | |
| FAM131A | family with sequence | Chr.3: | Hs.591307 | NM_001171093.1 | 64 | 4-5 | 498-561 |
| | similarity 131, | 184053717- | | | | | |
| | member A, | 184064063 | | | | | |
| | transcript variant 2 | | | | | | |
| FLJ10357 | Rho guanine | Chr.14: | Hs.35125 | NM_018071.4 | 102 | 16-17 | 3557-3658 |
| | nucleotide exchange | 21538527- | | | | | |
| | factor (GEF) 40 | 21558036 | | | | | |
| | (ARHGEF40) | | | | | | |
| FZD7 | frizzled homolog 7 | Chr. 2- | Hs.173859 | NM_003507.1 | 70 | 1-1 | 1-70 |
| | (Drosophila) | 202899310- | | | | | |
| | | 202903160 | | | | | |
| GLT8D1 | glycosyltransferase 8 | Chr.3: | Hs.297304 | NM_001010983.2 | 87 | 4-5 | 924-1010 |
| | domain containing 1, | 52728504- | | | | | |
| | transcript variant 3 | 52740048 | | | | | |
| HDAC9 | histone deacetylase 9, | Chr.7: | Hs.196054 | NM_001204144.1 | 69 | 11-12 | 1777-1845 |
| | transcript variant 6 | 18535369- | | | | | |
| | | 19036993 | | | | | |
| HSF2 | heat shock | Chr.6: | Hs.158195 | NM_004506.3 | 82 | 10-11 | 1324-1405 |
| | transcription factor 2, | 122720696- | | | | | |
| | transcript variant 1 | 122754264 | | | | | |
| Ki-67 | antigen identified by | Chr. 10- | Hs.689823 | NM_001145966.1 | 78 | 6-7 | 556-635 |
| | monoclonal antibody | 129894923- | | | | | |
| | Ki-67 | 129924655 | | | | | |
| KRAS | v-Ki-ras2 Kirsten | Chr. 12- | Hs.505033 | NM_004985.4 | 130 | 4-5 | 571-692 |
| | rat sarcoma viral | 25358180- | | | | | |
| | oncogene homolog | 25403854 | | | | | |
| LEO1 | Leo1, Paf1/RNA | Chr.15: | Hs.567662 | NM_138792.3 | 122 | 10-11 | 1753-1874 |
| | polymerase II | 52230222- | | | | | |
| | complex component | 52263958 | | | | | |
| | homolog (S. cerevisiae) | | | | | | |
| MORF4L2 | mortality factor 4 like | Chr.X: | Hs.326387 | NM_001142418.1 | 153 | 5-5 | 1294-1447 |
| | 2, transcript variant 1 | 102930426- | | | | | |
| | | 102943086 | | | | | |
| NAP1L1 | nucleosome assembly | Chr. 12- | Hs.524599 | NM_139207.2 | 139 | 16-16 | 1625-1764 |
| | protein 1-like 1 | 76438672- | | | | | |
| | | 76478738 | | | | | |
| NOL3 | nucleolar protein 3 | Chr.16: | Hs.513667 | NM_001185057.2 | 118 | 1-2 | 131-248 |
| | (apoptosis repressor | 67204405- | | | | | |
| | with CARD domain), | 67209643 | | | | | |
| | transcript variant 3 | | | | | | |
| NUDT3 | nudix (nucleoside | Chr.6: | Hs.188882 | NM_006703.3 | 62 | 2-3 | 500-561 |
| | diphosphate linked | 34255997- | | | | | |
| | moiety X)-type motif 3 | 34360441 | | | | | |
| OAZ2 | ornithine | Chr.15: | Hs.713816 | NM_002537.3 | 96 | 1-2 | 189-284 |
| | decarboxylase | 64979773- | | | | | |
| | antizyme 2 | 64995462 | | | | | |
| PANK2 | pantothenate kinase 2 | Chr.20: | Hs.516859 | NM_024960.4 | 126 | 4-5 | 785-910 |
| | | 3869486- | | | | | |
| | | 3904502 | | | | | |
| PHF21A | PHD finger protein | Chr.11: | Hs.502458 | NM_001101802.1 | 127 | 16-17 | 2241-2367 |
| | 21A, transcript | 45950870- | | | | | |
| | variant 1 | 46142985 | | | | | |
| PKD1 | polycystic kidney | Chr.16: | Hs.75813 | NM_000296.3 | 110 | 16-17 | 7224-7333 |
| | disease 1 (autosomal | 2138711- | | | | | |
| | dominant), transcript | 2185899 | | | | | |
| | variant 2 | | | | | | |
| PLD3 | phospholipase D | Chr.19: | Hs.257008 | NM_001031696.3 | 104 | 6-7 | 780-883 |
| | family, member 3, | 40854332- | | | | | |
| | transcript variant 1 | 40884390 | | | | | |
| PNMA2 | paraneoplastic | Chr. 8- | Hs.591838 | NM_007257.5 | 60 | 3-3 | 283-343 |
| | antigen MA2 | 26362196- | | | | | |
| | | 26371483 | | | | | |
| PQBP1 | polyglutamine binding | Chr.X: | Hs.534384 | NM_001032381.1 | 68 | 2-3 | 157-224 |
| | protein 1, transcript | 48755195- | | | | | |
| | variant 2 | 48760422 | | | | | |
| RAF1 | v-raf-1 murine | Chr. 3- | Hs.159130 | NM_002880.3 | 90 | 7-8 | 1186-1277 |
| | leukemia viral | 12625100- | | | | | |
| | oncogene homolog 1 | 12705700 | | | | | |
| RNF41 | ring finger protein 41, | Chr.12: | Hs.524502 | NM_001242826 | 72 | 2-3 | 265-336 |
| | transcript variant 4 | 56598285- | | | | | |
| | | 56615735 | | | | | |
| RSF1 | remodeling and | Chr.11: | Hs.420229 | NM_016578.3 | 60 | 7-8 | 2804-2863 |
| | spacing factor 1 | 77377274- | | | | | |
| | | 77531880 | | | | | |
| RTN2 | reticulon 2, | Chr.19: | Hs.47517 | NM_005619.4 | 87 | 9-10 | 1681-1766 |
| | transcript variant 1 | 45988550- | | | | | |
| | | 46000313 | | | | | |
| SMARCD3 | SWI/SNF related, | Chr.7: | Hs.647067 | NM_001003801.1 | 109 | 8-9 | 986-1094 |
| | matrix associated, actin | 150936059- | | | | | |
| | dependent regulator of | 150974231 | | | | | |
| | chromatin, subfamily | | | | | | |
| | d, member 3, | | | | | | |
| | transcript variant 3 | | | | | | |
| SPATA7 | spermato genesis | Chr.14: | Hs.525518 | NM_001040428.3 | 81 | 1-2 | 160-241 |
| | associated 7, | 88851988- | | | | | |
| | transcript variant 2 | 88904804 | | | | | |
| SST1 | somatostatin | Chr.14: | Hs.248160 | NM_001049.2 | 85 | 3-3 | 724-808 |
| | receptor 1 | 38677204- | | | | | |
| | | 38682268 | | | | | |
| SST3 | somatostatin | Chr.22: | Hs.225995 | NM_001051.4 | 84 | 2-2 | 637-720 |
| | receptor 3 | 37602245- | | | | | |
| | | 37608353 | | | | | |
| SST4 | somatostatin | Chr.20: | Hs.673846 | NM_001052.2 | 104 | 1-1 | 91-194 |
| | receptor 4 | 23016057- | | | | | |
| | | 23017314 | | | | | |
| SST5 | somatostatin | Chr.16: | Hs.449840 | NM_001053.3 | 157 | 1-1 | 1501-1657 |
| | receptor 5, | 1122756- | | | | | |
| | transcript variant 1 | 1131454 | | | | | |
| TECPR2 | tectonin beta-propeller | Chr.14: | Hs.195667 | NM_001172631.1 | 61 | 12-13 | 3130-3191 |
| | repeat containing 2, | 102829300- | | | | | |
| | transcript variant 2 | 102968818 | | | | | |
| TPH1 | tryptophan | Chr. 11- | Hs.591999 | NM_004179.2 | 145 | 1-2 | 73-219 |
| | hydroxylase 1 | 18042538- | | | | | |
| | | 18062309 | | | | | |
| TRMT112 | tRNA | Chr.11: | Hs.333579 | NM_016404.2 | 91 | 1-2 | 45-135 |
| | methyltransferase 11-2 | 64084163- | | | | | |
| | homolog (S. cerevisiae) | 64085033 | | | | | |
| VMAT1 | solute carrier family 18 | Chr. 8- | Hs.158322 | NM_003053.3 | 102 | 1-2 | 93-196 |
| | (vesicular monoamine), | 20002366- | | | | | |
| | member 1 | 20040717 | | | | | |
| VMAT2 | solute carrier family 18 | Chr. 10- | Hs.596992 | NM_003054.4 | 60 | 9-10 | 896-957 |
| | (vesicular monoamine), | 119000716- | | | | | |
| | member 2 | 119037095 | | | | | |
| VPS13C | vacuolar protein sorting | Chr.15: | Hs.511668 | NM_001018088.2 | 65 | 69-70 | 9685-9749 |
| | 13 homolog C | 62144588- | | | | | |
| | (S. cerevisiae), | 62352647 | | | | | |
| | transcript variant 2B | | | | | | |
| WDFY3 | WD repeat and FYVE | Chr.4: | Hs.480116 | NM_014991.4 | 81 | 64-65 | 10190-10270 |
| | domain containing 3 | 85590690- | | | | | |
| | | 85887544 | | | | | |
| ZFHX3 | zinc finger homeobox3, | Chr.16: | Hs.598297 | NM_001164766.1 | 68 | 5-6 | 886-953 |
| | transcript variant B | 72816784- | | | | | |
| | | 73092534 | | | | | |
| ZXDC | zinc finger C, | Chr.3: | Hs.440049 | NM_001040653.3 | 61 | 1-2 | 936-1001 |
| | transcript variant 2 | 126156444- | | | | | |
| | | 126194762 | | | | | |
| ZZZ3 | zinc finger, | Chr.1: | Hs.480506 | NM_015534.4 | 62 | 13-14 | 2909-2971 |
| | ZZ-type containing 3 | 78030190- | | | | | |
| | | 78148343 | | | | | |

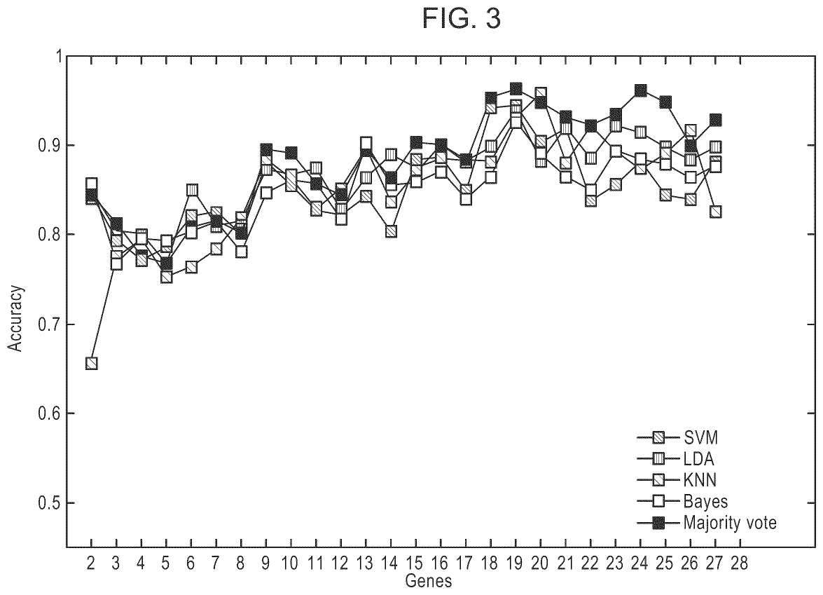

Delineation of Minimum Gene Set for Mathematically-Derived (MAARC-NET) Scoring System—Four classification algorithms (SVM, LDA, KNN, and Bayes) and a 10-fold cross-validation design were used to build a classifier for the identification of GEP-NETs in blood. See Modlin I, Drozdov I, Kidd M: The Identification of gut neuroendocrine tumor disease by multiple synchronous transcript analysis in blood. Plos One 2013, e63364. These classifiers were built on a training set and significantly up-regulated features between control and tumor cases were calculated by t-test. With reference to FIG. 3, an examination of the 51 genes featured in TABLE 2 identified that inclusion of at least 22 genes was sufficient to build an accurate (>0.85) classifier. FIG. 3 shows the prediction accuracy of each classifier algorithm using sequential addition of up to 27 significantly up-regulated genes (p<0.05) in the GEP-NET samples obtained using results of the 10-fold cross validation. The average accuracy of the SVM, LDA, KNN, and Bayes algorithms to distinguish GEP-NET from control blood samples using the sequentially added 27 genes was comparable-0.89 (0.85-1.0), 0.89 (0.86-0.93), 0.88 (0.85-0.93), and 0.86 (0.85-0.93) respectively. The “majority voting” combination of the four classifiers achieved an accuracy of 0.88. The at least 22 genes sufficient to build an accurate classifier were used to develop the MAARC-NET scoring system, and are featured in TABLE 3.

TABLE 3
Twenty Two Genes Included in the Mathematically-Derived
MAARC-NET Scoring System

| | Fold Change | p-value | Adjusted p-value |
|---|---|---|---|
| PNMA2 | 0.819515 | 6.74E−21 | 3.43E−19 |
| NAP1L1 | 0.662434 | 4.9E−18 | 1.25E−16 |
| FZD7 | 0.799858 | 3.82E−15 | 6.5E−14 |
| SLC18A2 | 0.524046 | 1.08E−12 | 1.37E−11 |
| NOL3 | 0.809571 | 7.22E−10 | 7.36E−09 |
| SSTR5 | 0.877322 | 1.64E−09 | 1.4E−08 |
| TPH1 | 0.459185 | 1.75E−07 | 1.27E−06 |
| RAF1 | 0.316509 | 1.54E−06 | 7.86E−06 |
| RSF1 | 0.530054 | 1.74E−06 | 8.07E−06 |
| SSTR3 | 0.555269 | 3.82E−06 | 1.62E−05 |
| SSTR1 | 0.493052 | 1.73E−05 | 6.81E−05 |
| CD59 | 0.26257 | 2.7E−05 | 9.82E−05 |
| ARAF | 0.228332 | 4.07E−05 | 0.000138 |
| APLP2 | 0.228153 | 4.42E−05 | 0.000141 |
| KRAS | 0.205822 | 9.92E−05 | 0.000298 |
| MORF4L2 | 0.319826 | 0.000169 | 0.000453 |
| TRMT112 | 0.269618 | 0.001125 | 0.002524 |
| MKI67 | 0.191245 | 0.003468 | 0.007074 |
| SSTR4 | 0.313807 | 0.003734 | 0.007324 |
| CTGF | 0.196845 | 0.007665 | 0.01303 |
| SPATA7 | 0.288625 | 0.01467 | 0.02338 |
| ZFHX3 | 0.13248 | 0.031354 | 0.045687 |

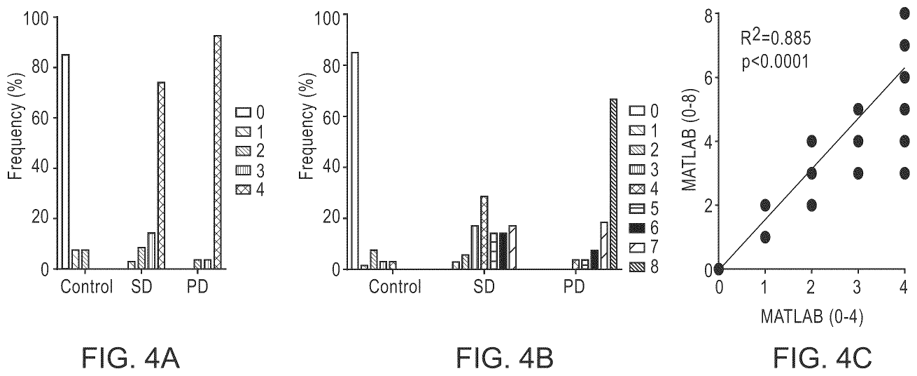

Refinement of Mathematically-Derived MAARC-NET Scoring System-Individual PCR-based gene expressions are included in a score. See Modlin I, Drozdov I, Kidd M, Plos One 2013. The score is based on a “majority vote” strategy and was developed from a binary classification system whereby a sample will be called “normal” and given a score of 0 or “tumor” and will be scored “1”. The score can range from 0 (four calls all “normal”) to 4 (four calls all “tumor”). Each “call” is the binary result (either “0” for normal or “1” for tumor) of one of four different learning algorithms: Support Vector Machine (SVM), Linear Discrimination Analysis (LDA), K-Nearest Neighbor (KNN), and Naïve Bayes (Bayes). Each of these four learning algorithms were trained on an internal training set including 67 controls and 63 GEP-NEN. In this training set, differentially expressed genes (control versus GEP-NEN) were identified as significant using a t-test. Based upon the training set, each of the learning algorithms were trained to differentiate between normal and tumor gene expression to within a level of significance of at least p<0.05. According to the majority voting strategy, those samples with less than 2 “normal” calls are classified as GEP-NEN. With reference to FIG. 4A, an audit of samples identified that 85% of controls exhibited a score of “0.” No tumors scored “0.” ROC analyses identified that a score of 2 was the cut-off for normal samples (controls) versus tumors (score≥2). This approach exhibited correct call rates of 91-97% with sensitivities and specificities of 85-98% and 93-97% for the identification of GEP-NETs in two independent sets. See Modlin I, Drozdov I, Kidd M, Plos One 2013.

These data were initially derived from a test data set of 130 samples (n=67 controls, n=63 NETs). Inherent in the test set are two classes of NETs-clinically defined as treated, stable disease (SD: n=35) and untreated, progressive disease (PD: n=28). The classification algorithm also segregated the tumor call into two units “treated” and “untreated.” The 0-4 binary classification was therefore amended to represent 3 possible calls for each particular sample: “normal”, “tumor (treated)” and “tumor (untreated)”.

A number of rules were implemented to generate an amended majority vote strategy. A call of “normal” was assigned a value of 0; a call of tumor “treated” was assigned a value of 1; a call of tumor “untreated” was assigned a value of 2. By way of example, if a sample results in four calls of “normal,” a value of 0 was assigned for each call, thereby totaling a score of 0. If a sample results in four calls of tumor “treated,” a value of 1 was assigned for call, thereby totaling a score of 4. If a sample results in four calls of tumor “untreated,” a “2” is assigned for each, thereby totaling a score of 8. Scores in the amended majority vote strategy can therefore range between 0 and 8.

Examination of the test dataset (n=130) was used to establish whether the amended majority vote-derived score could serve as a measure of “treatment” responses. Similarly to the published 0-4 score shown in FIG. 4A, the majority of NET patients exhibited an amended majority vote score>2 as shown in FIG. 4B. With reference to FIG. 4C, majority vote and amended majority vote scores were significantly related (R²=0.89, p<0.0001).

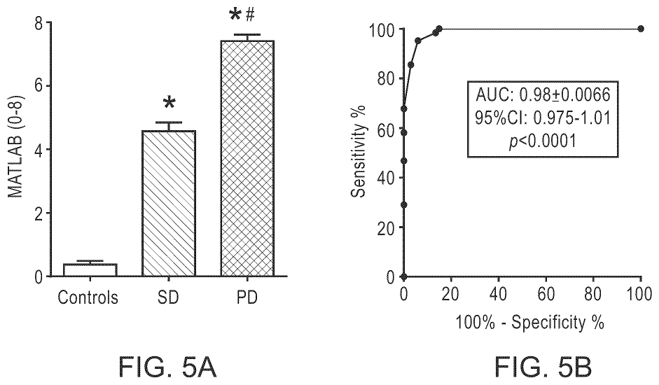

With reference to FIG. 5A, analysis of the data in the test set identified that an amended mathematically-derived score (0-8) was significantly elevated in tumors compared to controls and was highest in PD relative to SD.

With reference to FIG. 5B, a receiver operating characteristic (ROC) curve was generated of controls versus GEP-NETs (SD and PD combined). A ROC curve is a generalization of the set of potential combinations of sensitivity and specificity possible for predictors. A ROC curve is a plot of the true positive rate (sensitivity) against the false positive rate (1—specificity) for the different possible cut-points of a diagnostic test. FIG. 5B is a graphical representation of the functional relationship between the distribution of the sensitivity and specificity values in the test set and in a cohort of control samples. The area under the curve (AUC) is an overall indication of the diagnostic accuracy of (1) the amended mathematically-derived scores and (2) a receiver operating characteristic (ROC) curve. AUC may be determined by the “trapezoidal rule.” For a given ROC curve, the data points are connected by straight line segments, perpendiculars are erected from the abscissa to each data point, and the sum of the areas of the triangles and trapezoids so constructed is computed.

The ROC curve in FIG. 5B identifies that the amended mathematically-derived score may be utilized to differentiate between controls and GEP-NETs-exhibiting an AUC of >0.98, and a p<0.0001; *p<0.05 vs. controls; #p<0.05 vs. SD (2-tailed Mann-Whitney U-test).

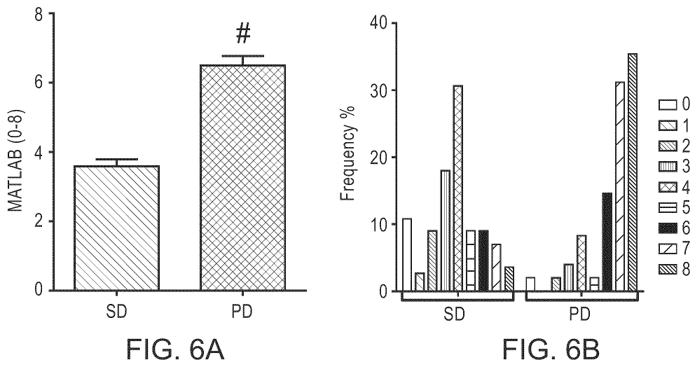

Amended mathematically-derived scores were subsequently examined in an independent set (SD: n=111, PD: n=48). With reference to FIG. 6A, the scores were significantly elevated in the independent set, exhibiting a p<0.0001. With reference to FIG. 6B, a frequency distribution plot of amended mathematically-derived scores in SD and PD patients confirmed that PD samples exhibited higher scores, with #p<0.0001 vs. SD (2-tailed Mann-Whitney U-test).

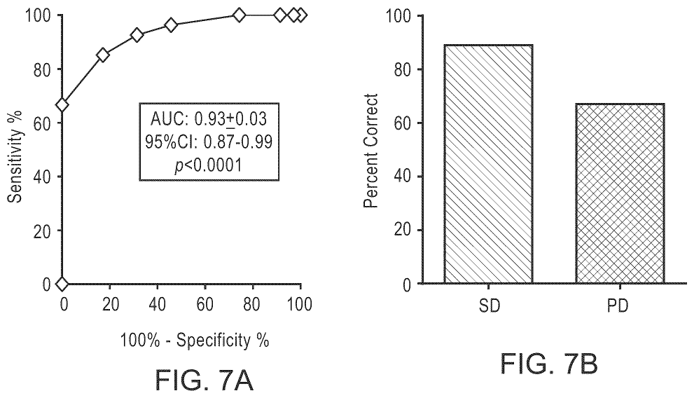

With reference to FIG. 7A, a second ROC curve was generated to determine whether the amended mathematically-derived score could be utilized to differentiate SD from PD. In the test set (SD: n=35, PD: n=28), the ROC analysis identified that the score could be used to differentiate PD from SD tumors with an AUC of 0.93. A score cutoff of >6.5 (i.e. a score of ≥7) had a sensitivity of 85% and 83% specificity for detecting PDs (Likelihood ratio: 4.97).

With reference to FIG. 7B, the utility of the amended mathematically-derived scoring system to differentiate between SD and PD in the independent set (n=111 SD, n=48 PD) was assessed. The percentage correctly called ranged between 70-90% using a cut-off of ≥7. For SD, 89% of NETs were correctly predicted using the cut-off of ≥7 while 67% of PD were correctly predicted. The performance metrics were: sensitivity=67%, specificity=89%, PPV=73% and NPV=86%. Accordingly, the data indicate that a mathematically-derived MAARC-NET score ranging from 0-8 has utility for discriminating between controls and GEP-NETs.

Application of Scoring System and Developing a Nomogram for “NETEST 1”—To differentiate between controls and NETs, a cut-off of ≥3 has a sensitivity of 95% and 94% specificity. The sensitivity can be improved to 98% using a cut-off of ≥2. To differentiate between SD and PD, a cut-off of ≥7 can be used (sensitivity of 85% and 83% specificity). The sensitivity can be improved to 96% using a cut-off of ≥5.

The mathematically-derived MAARC-NET scores therefore range from 0-2 (control); 2-5 (SD); and 5-8 (PD). These scores can be converted to a percentage as displayed in TABLE 4.

TABLE 4
Mathematically-Derived Scores Percentage

| | Mathematically-derived Score | | | |
|---|---|---|---|---|
| | 0-2 | 2-5 | 5-7 | 7-8 |
| Disease Nomogram | 0 | 0-55% | 55-75% | 75-100% |
| Score | | | | |
| | | Low | Moderate | High |

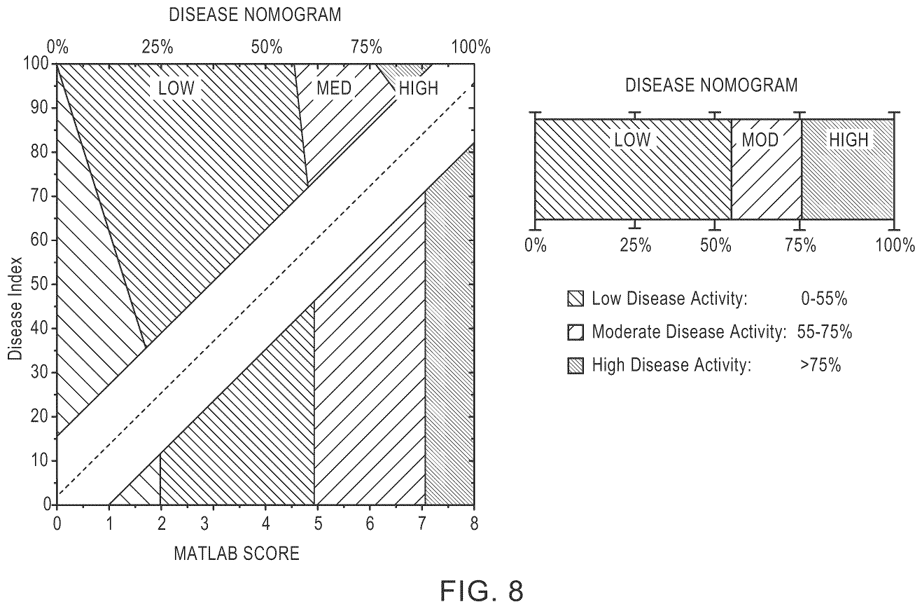

With reference to FIG. 8, the score percentages from TABLE 4 can be displayed within a nomogram representing “NETest 1.” The NETest 1 nomogram demonstrates how the amended mathematically-derived score is achieved and how it categorizes patients into different classes of GEP-NEN (no disease, stable disease, or progressive disease).

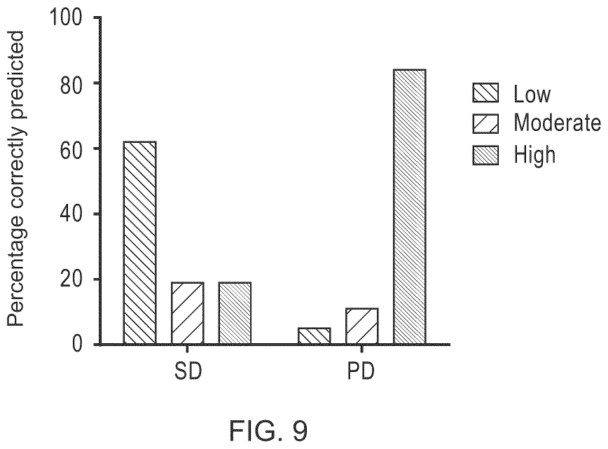

With reference to FIG. 9, the utility of the NETest 1 nomogram was assessed. Values for the correct predictions of SD and PD using the NETest 1 nomogram of FIG. 8 are shown. Overall, the NETest 1 nomogram identified 80% of SD patients as exhibiting low or moderate disease activity and 84% of PD patients as exhibiting high disease activity.

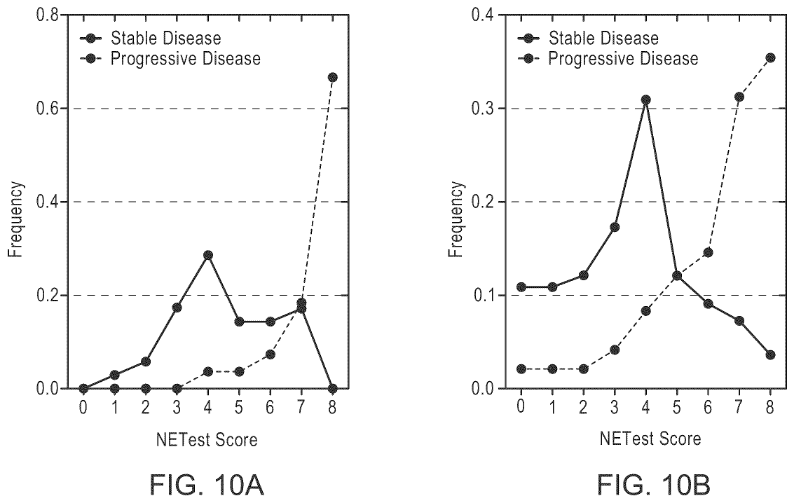

Application of Scoring System and Developing a Nomogram for “NETEST 2”-MAARC-NET-derivedNETest Scores (0-8) in patients clinically defined as either stable or progressive disease (best clinical judgment and/or imaging data) were examined. The frequency distribution of scores for each subtype in both the test set (FIG. 10A) or the independent set (FIG. 10B) demonstrate that SD patients have a median NETest value of 4 and PD patients range from 7-8. However, SD patients can exhibit MAARC-NET-derived scores>4 while PD can exhibit scores<7.

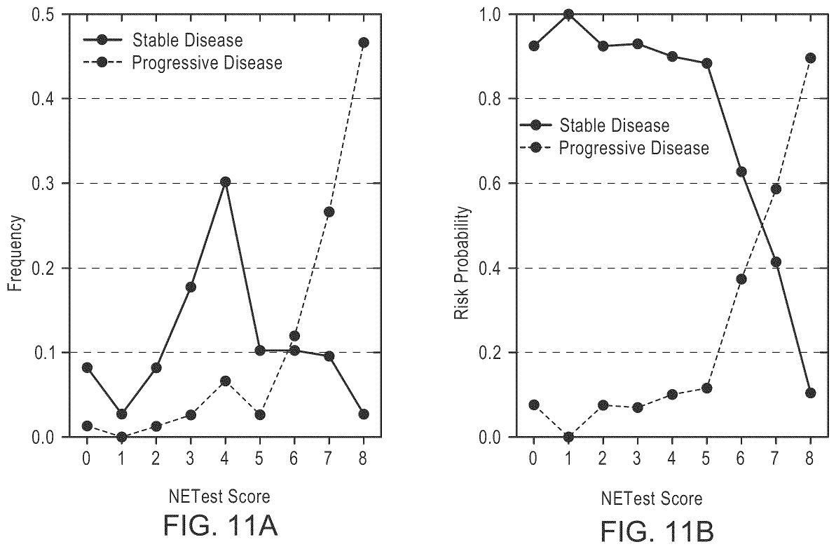

An assessment of the complete patient group (test set+independent set) demonstrated that the highest frequency SD score was 4 (30%-FIG. 11A), while 46% of PD had a score of 8 (FIG. 11A). A risk probability assessment identified that NETest scores ranging between 0-5 were associated with SD with a ≥90% certainty (FIG. 11B). A score of 8 was most likely PD (>90%). However, scores of 6 and 7 could not accurately differentiate SD versus PD.

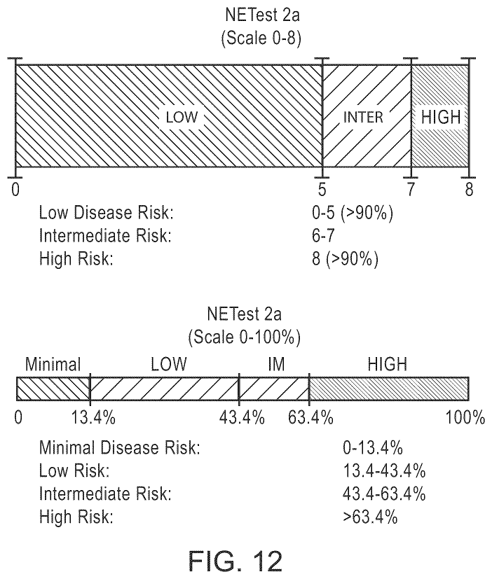

Based on these results from FIGS. 11A and 11B, the NETest 1 nomogram from FIG. 8 can be updated to include risk values. The NETest 2a nomogram of FIG. 12 includes the NETest with the inclusion of score and risk categorizations.

To upgrade the risk assessment NETest 2a nomogram, individual gene expression in SD and PD samples may be evaluated. The genes that were most differentially expressed in SD and PD samples were identified and used in decision trees to generate the rules for defining whether a NETest score was SD or PD. This approach provides the basis for NETest 2.

A NETest score of 5 has a >90% chance of identifying an SD sample (as shown in FIGS. 11A-11B and 12). Comparisons of the individual 51 gene expression profiles between patients scored as 5 (SD versus PD) identified expression of SMARCD3 and TPH1 as candidate differentiation markers. Using the rule:
If SMARCD3<0.13 and TPH1<4 then call PD.

This allowed for 100% accuracy in defining progressive disease.

A NETest score of 6 has a ˜50% chance of differentiating SD from PD samples. Gene expression profile analysis identified VMAT1 and PHF21A as candidates. A ROC analysis defined the AUCs for each to differentiate PD from SD to be:

VMAT1: ROC = 0.835
PHF21A: ROC = 0.733
Using the rule:
If VMAT1≥0 and PHF21A<1.2 then SD
If VMAT1≥0 and PHF21A≥1.2 then PD

This allowed for 100% accuracy in defining progressive disease and 90% accuracy in defining SD. The overall accuracy was 93%.

A NETest score of 7 has a ˜50% chance of differentiating SD from PD samples. As for NETest scores of 6, gene expression profile analysis identified both VMAT1 and PHF21A as candidates. A ROC analysis defined the AUCs for each to differentiate PD from SD to be:

VMAT1: ROC = 0.835
PHF21A: ROC = 0.733
Using the rule:
If VMAT1≥0 and PHF21A>1 then SD
If VMAT1≥0 and PHF21A≤1 then PD

This allowed for a 100% accuracy for defining progressive disease and 95% accuracy for SD. The overall accuracy was 97.5%.

A NETest score of 8 has a ≥90% chance of identifying a sample as PD. Expression of ZZZ3 was identified as a candidate. A ROC analysis defined the AUC for this gene to be 1.0.

Using the rule:
If ZZZ3≤14 then PD

This allowed for a 100% accuracy for defining progressive disease and differentiating from SD.

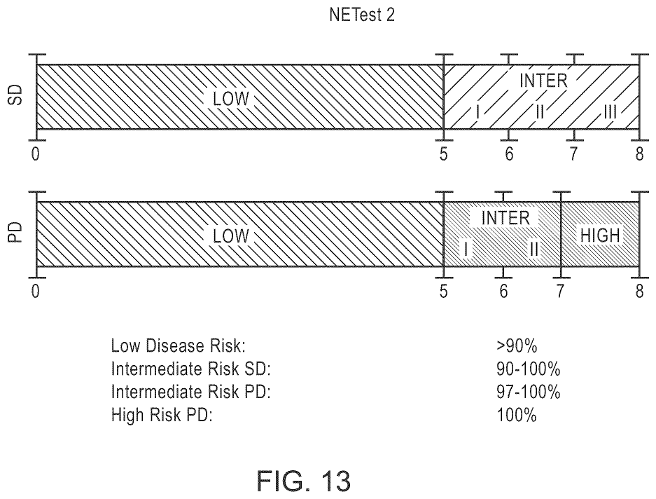

With reference to FIG. 13, this individual gene expression information was used to finalize the “NETest 2” nomogram, which provides an accurate disease categorization profile for the patient. The combination of NETest scores and individual gene expression information used in the NETest 2 nomogram of FIG. 13 is further detailed in TABLE 5.

TABLE 5
NETEST 2 Nomogram Information

| | | Accuracy |
|---|---|---|
| Low risk stable disease | NETest score 0-5 | 90-100% |
| Intermediate risk stable | NETest score 6 (low | 90-100% |
| disease (I) | PHF21A) | |
| Intermediate risk stable | NETest score 7 (high | 95-100% |
| disease (II) | PHF21A) | |
| Intermediate risk stable | NETest score 8 (high ZZZ3) | 100% |
| disease (III) | | |
| Intermediate risk progressive | NETest score 6 (high | 100% |
| disease (I) | PHF21A) | |
| Intermediate risk progressive | NETest score 7 (low | 97.5-100% |
| disease (II) | PHF21A) | |
| High risk progressive | NETest score 8 (low ZZZ3) | 100% |
| disease | | |

Defining Clinically Relevant Genes—To further refine the scoring system, gene cluster expression was examined and algorithms were developed to capture the information. Individual gene clusters incorporate biological information that may augment the mathematically-derived MAARC-NET scoring systems. One focus may be given to literature-curated gene clusters which are included in TABLE 6.

TABLE 6
Genes included in each Cluster

| Cluster Name | Genes |
|---|---|
| Proliferome | Ki67, NAP1L1, NOL3, TECPR2 |
| Growth Factor Signalome | ARAF1, BRAF, KRAS, RAF1 |
| Metabolome | ATP6V1H, OAZ2, PANK2, PLD3 |
| Secretome I (General) | PNMA2, VMAT2 |
| Secretome II (Progressive) | PQBP1, TPH1 |
| Epigenome | MORF4L2, NAP1L1, PQBP1, RNF41, RSF1, |
| | SMARCD3, ZFHX3 |
| Apoptome | BNIP3L, WDFY3 |
| Plurome | COMMD9 |
| SSTRome | SSTR1, SSTR3, SSTR4, SSTR5 |

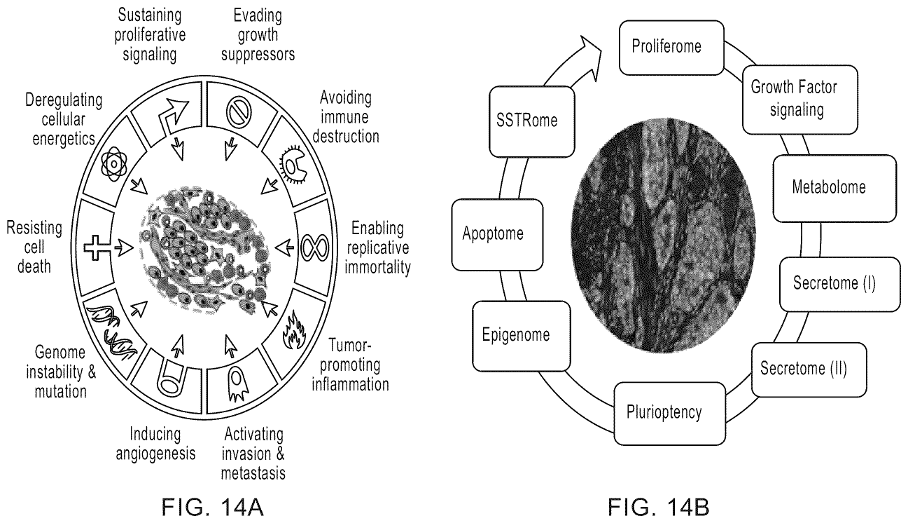

With reference to FIG. 14A, the Hallmarks of Neoplasia are illustrated, including the delineation of tumor (adenocarcinoma)-derived hallmarks. With reference to FIG. 14B, the NET hallmarks based on the Hanahan and Weinberg classifications are illustrated.

Values for the nine clusters represented in FIGS. 14A-14B were derived from gene addition. In addition to the gene clusters, two algorithms were also assessed:
1) the “PDA” algorithm, which included a summation of the proliferome, signalome, secretome II, plurome and epigenome (the PDA algorithm is also referred to as Progressive Diagnostic I);
2) the “NDA” algorithm, which included expression of 15 genes associated with disease: these included ARAF1, BRAF, KRAS, RAF1, Ki67, NAPILI, NOL3, GLT8D1, PLD3, PNMA2, VMAT2, TPH1, FZD7, MORF4L2 and ZFHX3 (the NDA algorithm is also referred to as Progressive Diagnostic II). Genes were summated and an averaged value was derived.

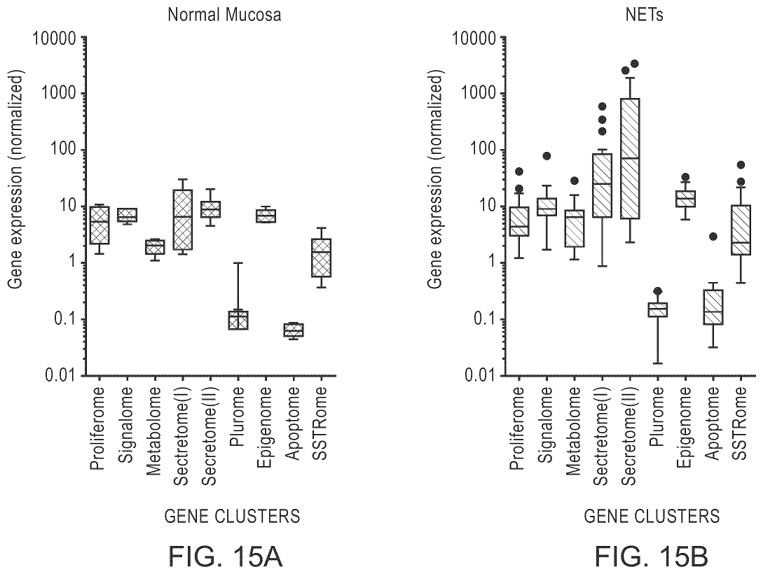

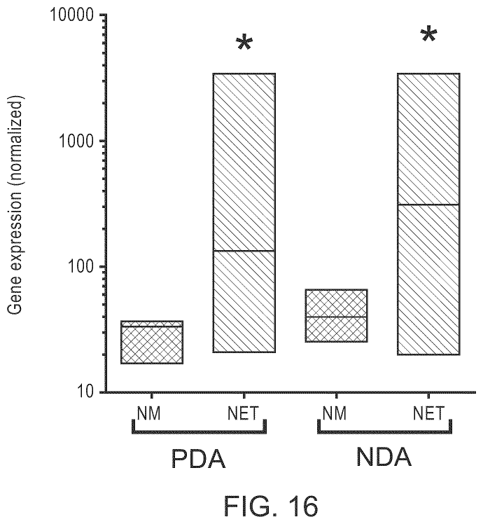

Prior to assessing the value of the nine gene clusters and two algorithms in blood samples, their expression in NET tumor tissue was assessed to confirm that these were NET-relevant. With reference to FIGS. 15B and 15A, respectively, expression in 22 NETs may be compared to expression in normal mucosa (n=10). Assessment identified that seven of the nine clusters were specific to NETs (in comparison to normal mucosa). In particular, expression of the signalome, metabolome, secretome (I) and (II), epigenome, apoptome and SSTRome were elevated in NETs (p<0.05). Genes in the apoptome were decreased in NETs, while the proliferome was not different between NETs and normal mucosa. With respect to the algorithms, FIG. 16 shows that each of the PDA and NDA were significantly increased (p<0.05) in NET tumor tissue compared to normal mucosa.

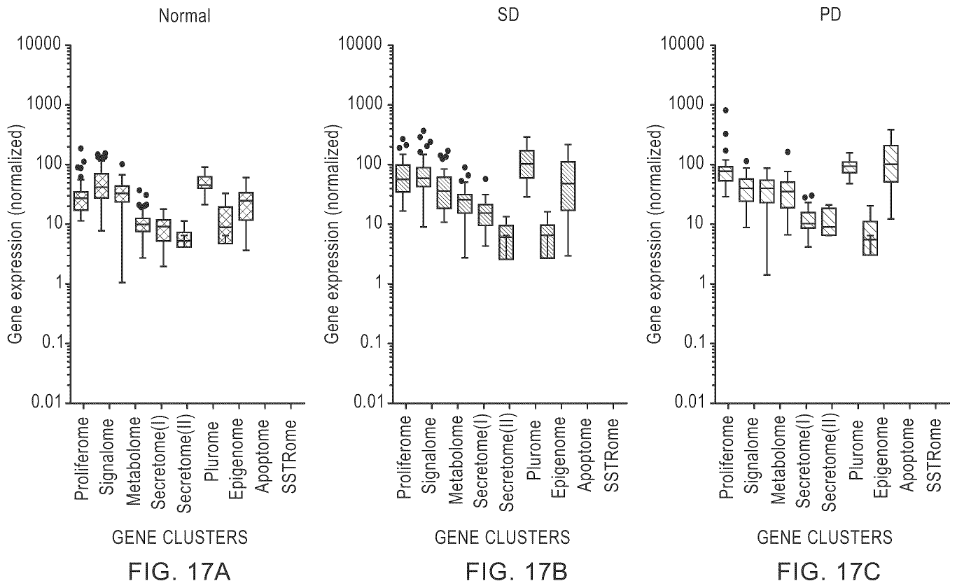

Thereafter, the expression of each of the clusters was assessed in blood samples. We examined the test (n=130) set and evaluated whether expression they were related to SD or PD. Significant differences were noted in gene expression between controls and SD/PD, as shown in FIGS. 17A-17C and TABLE 7.

TABLE 7
Gene Clusters and Clinical Outcome

| Cluster Name | Con vs SD | Con vs PD | SD vs PD |
|---|---|---|---|
| Proliferome | p < 0.05 | p < 0.05 | ns |
| Growth Factor Signalome | p < 0.05 | ns | p < 0.05 |
| Metabolome | ns | p < 0.05 | ns |
| Secretome I (General) | p < 0.05 | p < 0.05 | p < 0.05 |
| Secretome II (Progressive) | p < 0.05 | p < 0.05 | p < 0.05 |
| Epigenome | ns | p < 0.05 | p < 0.05 |
| Apoptome | p < 0.05 | p < 0.05 | ns |
| Plurome | p < 0.05 | p < 0.05 | ns |
| SSTRome | p < 0.05 | p < 0.05 | p < 0.05 |

ns = not significant
Two-tailed Mann-Whitney U-test

These data demonstrate that gene clusters can be used to differentiate SD and PD from controls as well as identify differences between SD and PD.

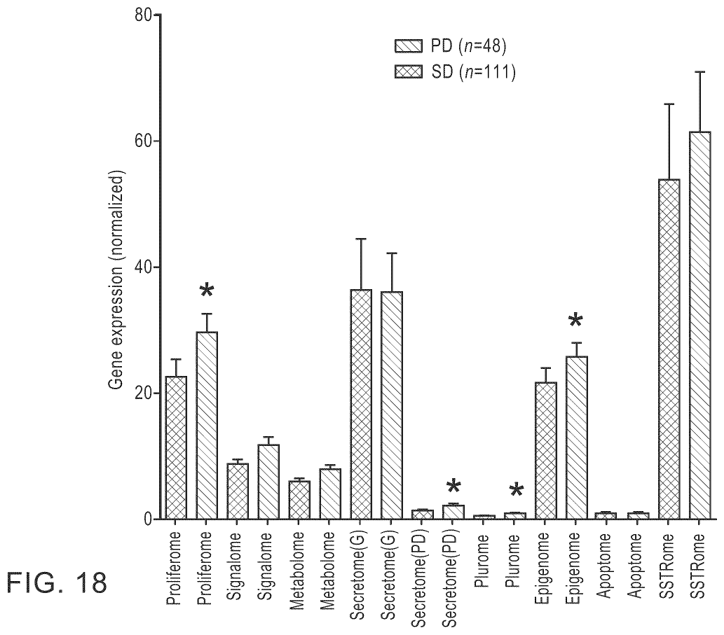

With reference to FIG. 18, gene cluster results were examined in the independent set (n=159), evaluating each of the clusters in SD vs PD. In the independent set, the proliferome, secretome (II), plurome and epigenome were significantly increased.

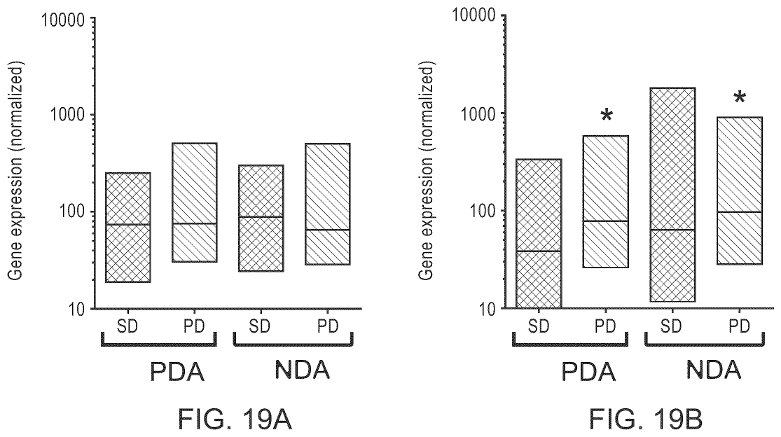

Next the PDA and NDA were evaluated in each of the two datasets (independent and test sets). With reference to FIG. 19A, no significant differences were identified between SD and PD for either of the two algorithms in the test set. With reference to FIG. 19B, each of the PDA and NDA were elevated in the independent set.

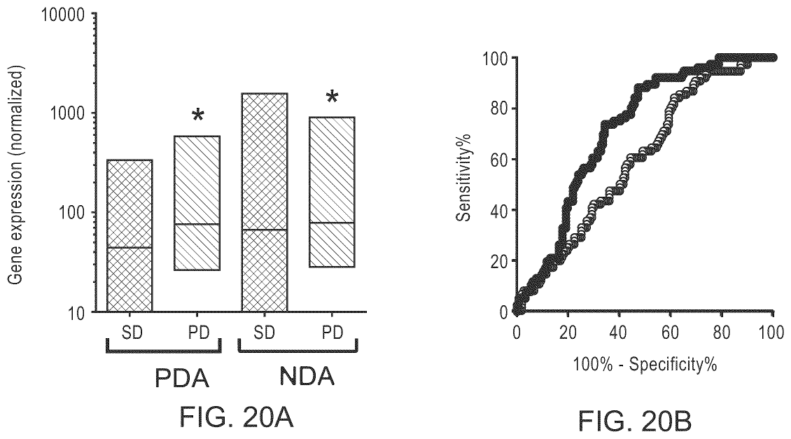

Next each of the algorithms were included in a combined set (test+independent: n=222) and their utility to predict SD versus PD was evaluated. With reference to FIG. 20A, both PDA and NDA were elevated in PD compared to SD in the combined sets. With reference to FIG. 20B, a ROC analysis identified the following parameters for PDA and NDA listed in TABLE 8.

TABLE 8
ROC Analysis Parameters, PDA and NDA in Combined Set

| | PDA | NDA |
|---|---|---|
| AUC | 0.72 ± 0.034 | 0.6 ± 0.038 |
| 95% CI | 0.652-0.785 | 0.525-0.675 |
| p-value | <0.0001 | 0.014 |
| ROC cut-off | 58 | 74 |

Two additional algorithms based on gene cluster expression differences in the test (TDA) and independent (IDA) set were evaluated. TDA included a summation of gene clusters significantly different between SD and PD in the test set.

These included TDA: Secretome (I), Plurome and SSTRome (the TDA algorithm is also referred to as Progressive Diagnostic III); and
IDA: Proliferome, secretome (II), plurome and epigenome (the IDA algorithm is also referred to as Progressive Diagnostic IV).

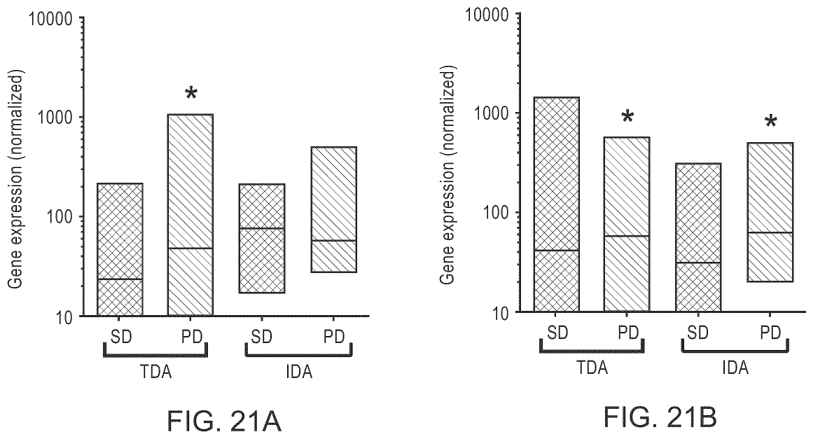

Each of the algorithms in the test set and independent set were evaluated. With reference to FIG. 21A, TDA was significantly elevated in PD compared to SD in the test set. With reference to FIG. 21B, both TDA and IDA algorithms were significantly elevated in the independent set.

Next, a ROC analyses with both algorithms in the combined dataset was performed. The ROC analysis identified the following parameters for TDA and IDA listed in TABLE 9.

TABLE 9
ROC Analysis Parameters, TDA and IDA in Combined Set

| | TDA | IDA |
|---|---|---|
| AUC | 0.62 ± 0.04 | 0.70 ± 0.034 |
| 95% CI | 0.542-0.698 | 0.637-0.770 |
| p-value | 0.003 | <0.001 |
| ROC-cut-off | >43 | >46 |

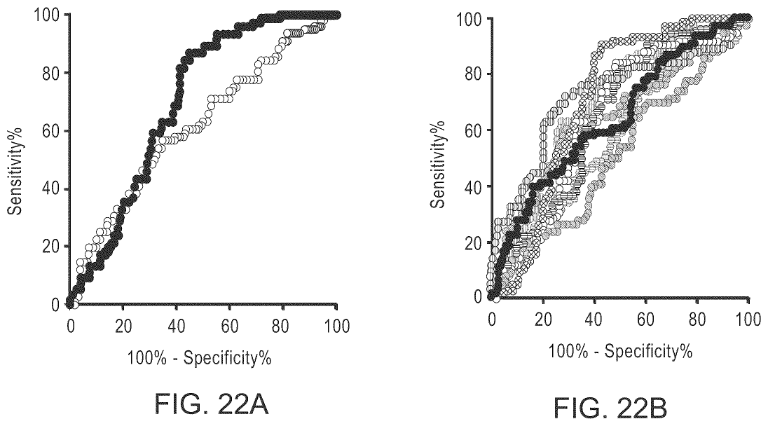

Algorithm-generated ROC curves of TDA and IDA for differentiating between SD and PD are shown in FIG. 22A. Algorithm-generated ROC curves for each of the clusters for differentiating between SD and PD are shown in FIG. 22B. The ROC curves in FIGS. 22A and 22B demonstrate that AUCs range from 0.51 (GF signalome) to 0.72 (plurome) for the differentiation of SD and PD.

Accordingly, individual gene cluster expression and algorithms that capture this information contain biologically relevant information that correlates with clinical observations. These provide the basis for defining clinically relevant MAARC-NET scoring systems.

Demonstration of Clinical Utility of NETEST Genes—The clinical utility of NETest scores, as well as the scores from pertinent gene clusters and algorithms, will now be defined. An examination of how surgical removal of a NET altered the circulating gene signature was performed to demonstrate how the test will have utility as a measure of the completeness of surgical therapy.

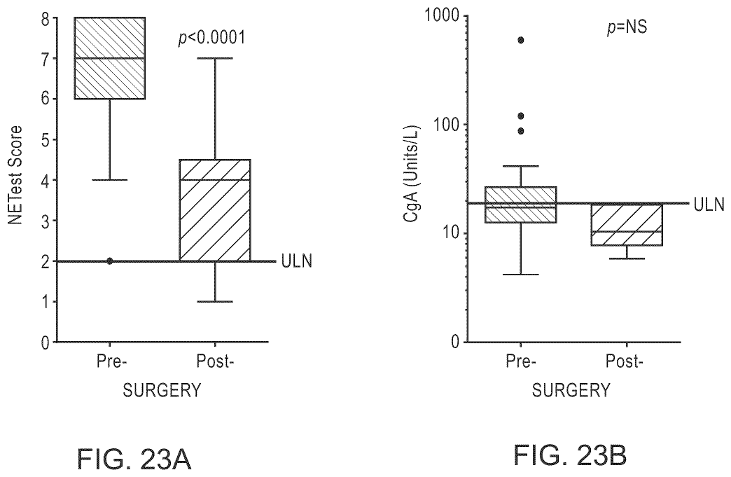

Parameters in 29 surgically treated patients prior to surgery and >1 month post-surgery was examined. As a group, MAARC-NET scores were significantly decreased (p<0.0001) from a mean of 6.58±1.48 to 3.65±1.6, as shown in in FIG. 23A. Chromogranin A (CgA), a gene used in a prior known single biomarker assay for NETs, was not significantly decreased (58.5±137.9 ng/ml vs. 55.25±154.8), as shown in FIG. 23B.

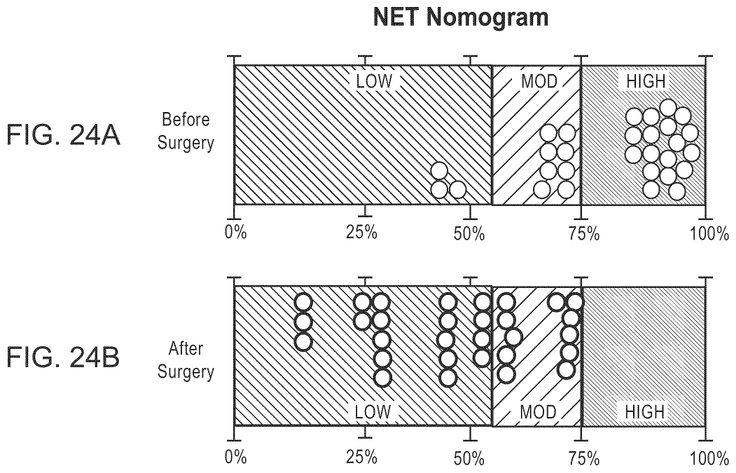

An examination of how NETest 1 performed, i.e. changes in NETest score pre- and post-surgical therapy, is included in FIGS. 24A-24B. Prior to surgery, 62% of patients were included in the high disease category; after surgery this was 0% (χ²=24, p=5×10⁻⁸).

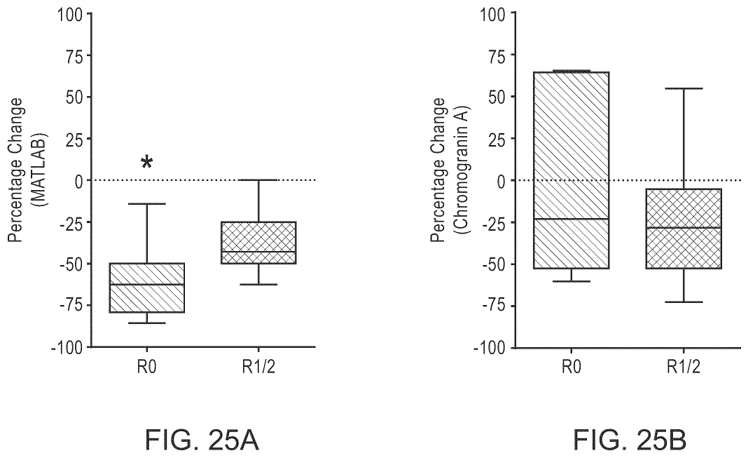

An alternative assessment of how surgery affected disease status is provided by the percentage changes in surgical approaches-no evidence of residual disease (R0) versus evidence of residual disease including metastases. With reference to FIG. 25A, levels for the MAARC-NET score were significantly decreased (p<0.003) in the R0 group (complete resection) compared to the R1/R2 group (incomplete resection).

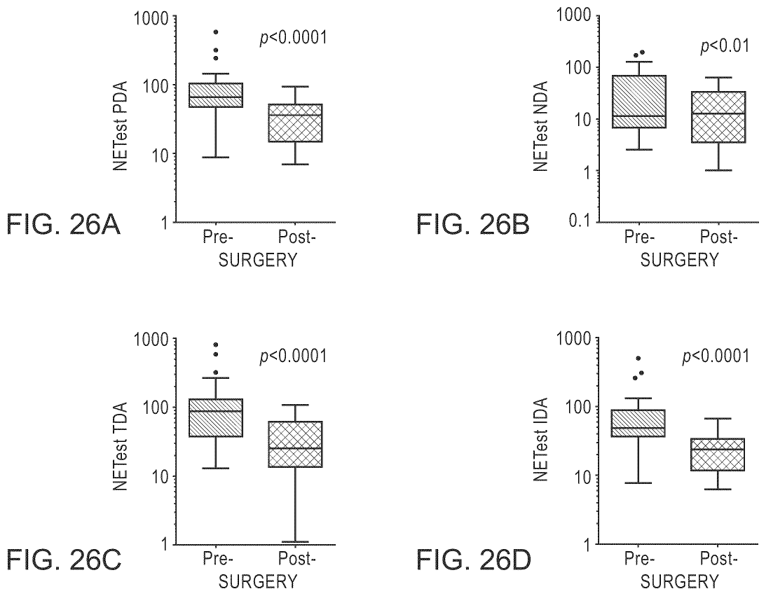

To better define the role of surgery each of the four algorithms were examined. Significant decreases were identified (post-surgery) in PDA (99.3±21 vs. 41.1±7.5, p<0.0001; FIG. 26A), NDA (45.8±10.3 vs. 29.6±7.8, p<0.01; FIG. 26B), TDA (133.3±32.3 vs. 43.8±9.3, p<0.0001; FIG. 26C) and IDA (86.1±19.3 vs. 34.1±7.2, p<0.0001; FIG. 26D).

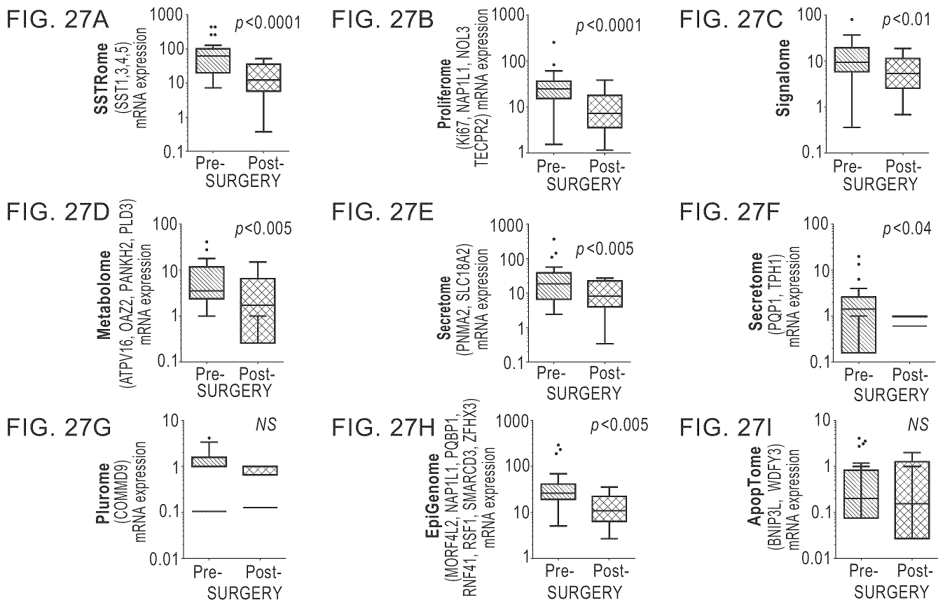

With reference to FIGS. 27A-27I, an examination of individual clusters identified significant decreases in the SSTRome, proliferome, GF signalome, metabolome, secretome I/II and the epigenome pre- and post-surgery.

With reference to TABLE 10, surgical removal of the tumor tissue was associated with decreases in circulating gene expression to levels not different to or below ROC cut-off values for SD for each of the four algorithms and for 6 of the 9 gene clusters.

TABLE 10
Relationship Between Surgical Excision, Gene Clusters
and Each of the Algorithms

| Algorithm/ | | | | | ROC for |
|---|---|---|---|---|---|
| Cluster | p-value | Change | Pre-surgery | Post-surgery | SD |
| NDA | 0.009 | ↓ | 45 | 30 | <74 |
| PDA | <0.0001 | ↓ | 99 | 41 | <58 |
| TDA | <0.0001 | ↓ | 133 | 44 | <74 |
| IDA | <0.0001 | ↓ | 86 | 34 | <46 |
| SSTRome | <0.0001 | ↓ | 93 | 23 | <25.5 |
| Proliferome | <0.0001 | ↓ | 34 | 15 | <20 |
| GF Signalome | 0.009 | ↓ | 14.8 | 8 | <9 |
| Metabolome | 0.004 | ↓ | 8.2 | 6.8 | <6.5 |
| Secretome (I) | 0.004 | ↓ | 39.2 | 19.5 | <11 |
| Secretome (II) | 0.04 | ↓ | 2.4 | 0.85 | <1.6 |
| Plurome | NS | ↔ | 0.8 | 0.8 | <0.9 |
| Epigenome | 0.005 | ↓ | 48.7 | 17.7 | <2.3 |
| Apoptome | NS | ↔ | 0.72 | 0.84 | >0.5 |

All patients who had surgery can be considered as exhibiting progressive/active disease. Following surgery, the scores or algorithms were indicative of progressive disease in 3-7 of the twenty-nine patients (10-24%) depending on the algorithm used.

Surgery significantly reduced the circulating tumor signature and can provide evidence for the degree both of tumor removal as well as for evidence of residual active disease.

The clinical utility of the test therefore is defined by the examination of scores, algorithms and clusters and evaluation in comparison to pre-surgical bloods. Evidence of elevated expression of e.g., PDA or proliferome in post-surgical samples is indicative of residual progressive (highly active disease).

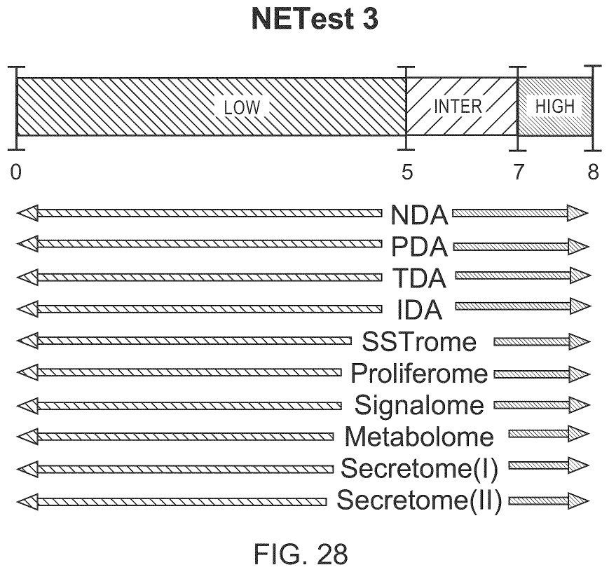

With reference to FIG. 28, a NETest 3 nomogram is illustrated with the inclusion of surgically-relevant algorithms and gene clusters. A combination score, as well as alterations in gene clusters e.g., a significant increase in the proliferome, will be indicative of disease regrowth following surgery. Of note, is that while post-operative imaging identified disease in n=1 (10%) of the R0 patients, elevated gene scores were evident in 6 (60%) at 1 month. Subsequently, two R0 individuals developed positive imaging at 6 months.

Effect of Standard Drug Therapies on Circulating NET Signature—The efficacy of a standard pharmacological therapy for NETs, somatostatin (used to treat>80% of patients), was evaluated on the circulating NET signature. Signatures were evaluated in patients treated with a somatostatin analog who were considered as either SD (n=63) or PD (n=26) by imaging and best clinical judgment. Those patients who were SD on somatostatin analogs were considered to be stable-treated patients, while those patients who were PD on somatostatin analogs were considered to be failing therapy.

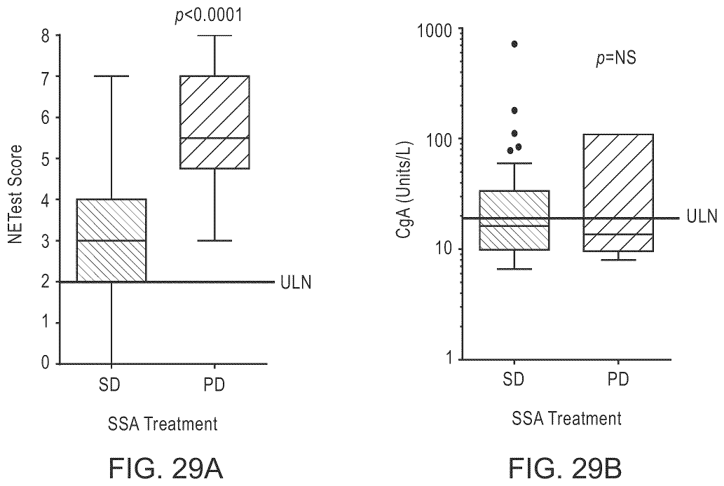

With reference to FIG. 29A, MAARC-NET scores were significantly lower in the SD group than those failing therapy: 3.33±0.21 vs 5.77±0.3 (p<0.001). With reference to FIG. 29B, Chromogranin A was not significantly different in the two groups (44.7±17.2 ng/ml vs. 102.4±58.7).

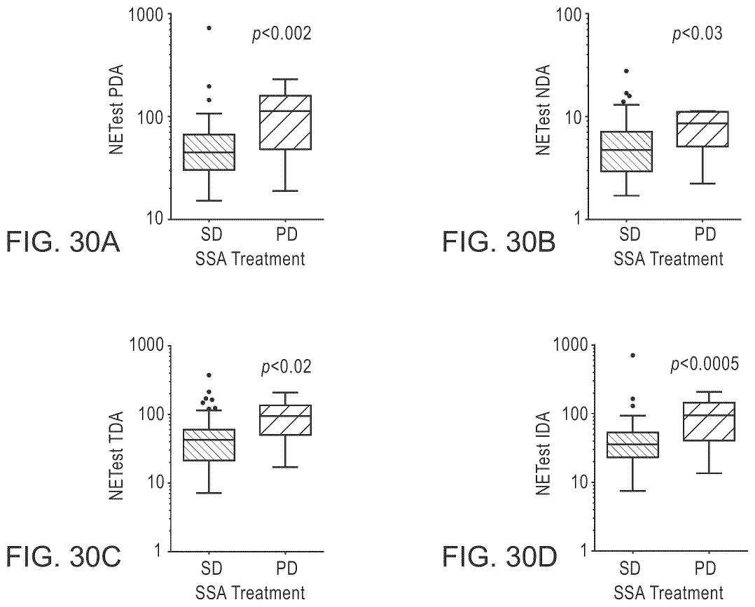

An assessment of the algorithms demonstrated significant differences in each of them in SD compared to PD. Specifically, PDA (62.8±11.4 vs. 153.9±36.2, p<0.002; FIG. 30A), NDA (6±0.6 vs. 13.5±3, p<0.03; FIG. 30B), TDA (56.8±7.4 vs. 154±37.2, p<0.02; FIG. 30C) and IDA (51.7±11.1 vs. 140.5±36, p<0.0005; FIG. 30D).

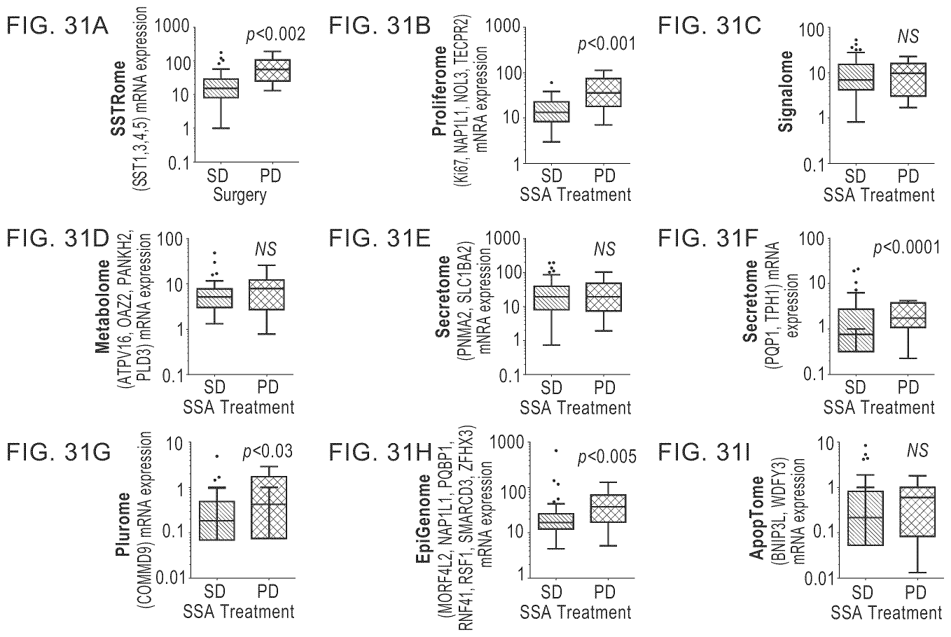

With reference to FIGS. 31A-31I, examination of individual clusters identified that the SSTRome, proliferome, secretome II, plurome and the epigenome were significantly lower in the SD group relative to the PD group.

These data demonstrate that patients who exhibit progressive disease despite somatostatin analog (SSA) therapy exhibit increases in the MAARC-NET score, as well as each of the four algorithms and specific gene clusters including an increase in proliferation, as well as the epigenome. One mechanism to evaluate whether the SSA treatment is effective therefore is to evaluate whether scores for these parameters alter. However, given the overlap in each of these parameters between the SD and PD groups, it would be helpful to better define the PD group. To do this, the expression may be compared of the circulating signature in those failing therapy to that in controls. The hypothesis behind this approach was that an effective therapy (i.e. SD) would normalize the signatures. The corollary is that PD will be significantly different to normal. To establish this, ROC analyses were used to examine normal circulating transcripts and compared to PD. All four algorithms were examined as well as the gene clusters.

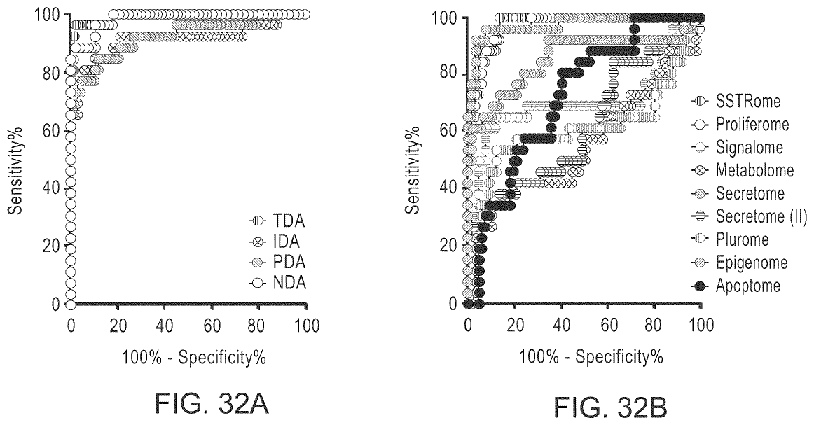

With reference to FIGS. 32A-32B, analysis of the data identified that algorithms (FIG. 32A) and selected clusters (FIG. 32B) differentiated controls from PD treated with SSAs. Data for the individual clusters are included in TABLE 11.

TABLE 11
Relationship between Gene Clusters and each of the Algorithms
for those Failing SSA Therapy and Controls

| Algorithm/ | | | | ROC for |
|---|---|---|---|---|
| Cluster | AUC | 95% CI | p-value | PD |
| NDA | 0.98 ± 0.01 | 0.965-1.00 | <0.0001 | >3 |
| PDA | 0.92 ± 0.04 | 0.851-0.994 | <0.0001 | >40 |
| TDA | 0.99 ± 0.01 | 0.975-1.01 | <0.0001 | >29 |
| IDA | 0.91 ± 0.04 | 0.828-0.998 | <0.0001 | >31 |
| SSTRome | 0.98 ± 0.01 | 0.95-1 | <0.0001 | >22 |
| Proliferome | 0.97 ± 0.02 | 0.94-1 | <0.0001 | >14 |
| GF Signalome | 0.71 ± 0.07 | 0.564-0.855 | <0.002 | >5 |
| Metabolome | 0.56 ± 0.07 | 0.41-0.7 | NS | <8 |
| Secretome (I) | 0.98 ± 0.02 | 0.944-1 | <0.0001 | >4 |
| Secretome (II) | 0.62 ± 0.07 | 0.486-0.759 | NS | >1.6 |
| Plurome | 0.61 ± 0.08 | 0.454-0.763 | NS | <0.7 |
| Epigenome | 0.86 ± 0.05 | 0.756-0.962 | <0.0001 | >16 |
| Apoptome | 0.73 ± 0.06 | 0.618-0.834 | <0.001 | <0.95 |

Based on the data in TABLE 11, NDA and TDA were examined as well as the SSTRome, Proliferome, and Secretome (I) in the SD cases to evaluate whether these parameters correlated with clinical assessments of therapeutic efficacy.

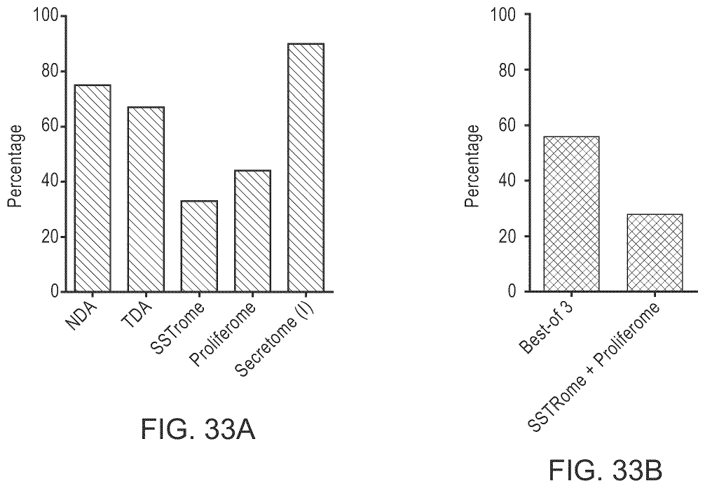

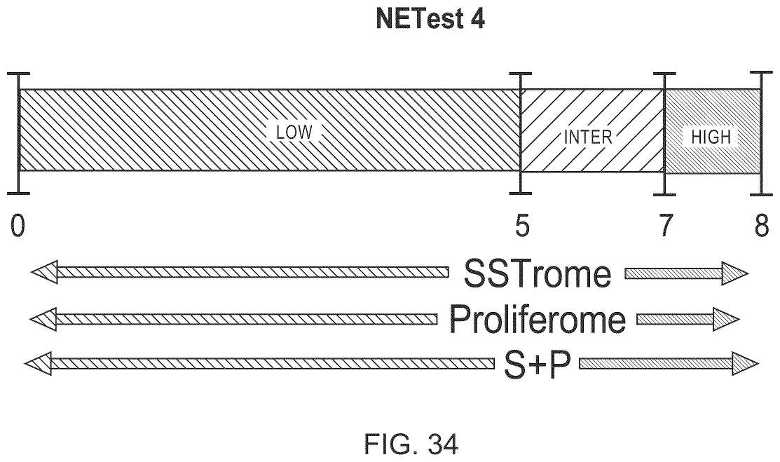

An assessment of individual algorithms or gene clusters identified that samples would be categorized as exhibiting disease in 33-75% of cases (FIG. 33A). In comparison to a best of 3 score (56%) a combination of elevations in the SSTRome and Proliferome resulted in the lowest number of cases (28%) predicted as exhibiting progressive disease (FIG. 33B). With reference to FIG. 34, the nomogram for somatostatin analog treated patients, named “NETest 4,” therefore includes the MAARC-NET score as well as the SSTRome, proliferome and their combination.

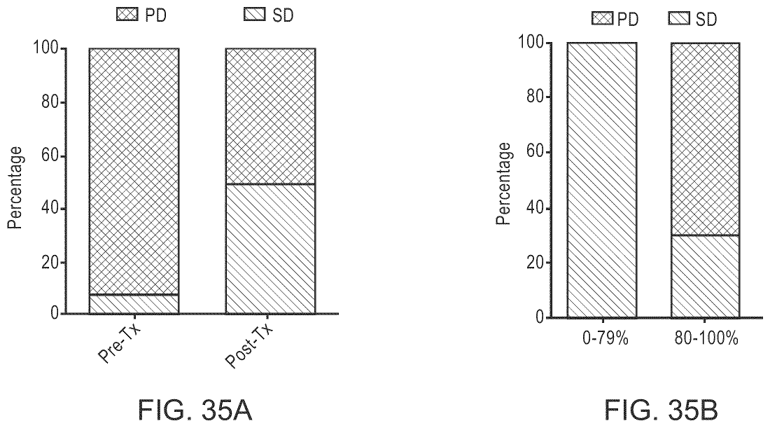

Utility of NETEST and Gene Expression for the Prediction of Somatostatin Analog Efficacy—To evaluate the utility of the NETest in therapy, the relationship between SSAs and clinically defined outcomes (per RECIST criteria) were evaluated. Samples were collected both pre-therapy as well as monthly in twenty-eight patients. Imaging was available to stage and categorize disease patterns pre- and during therapy (up to 12 months follow-up). In this prospective sample set, SSA resulted in a significant reduction in the number of patients with progressive disease (FIG. 35A).

Scores were also determined in blood samples collected prior to as well as monthly during SSA treatment to evaluate whether early alterations were predictive of outcome, i.e., response to therapy.

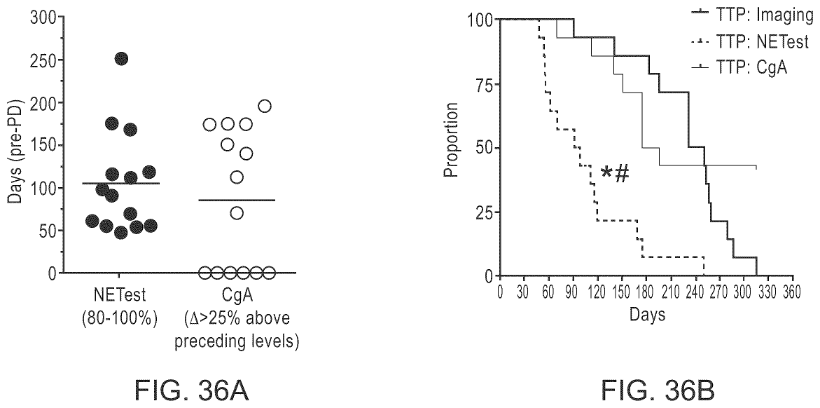

With reference to FIG. 35B, the results identify that elevated NETest scores (80-100% activity) measured at any time point during therapy were predictive of therapeutic responsiveness. With reference to FIG. 36A, a significant rise in the NETest (80-100%) occurred from 48-252 days (mean=105 days) prior to the detection of clinically significant disease (PD). The mean time for CgA was 70 days (range: 0-196 days). The NETest was more informative, occurring at an earlier time (p=0.04), and in more patients (high activity was noted in 100%) than CgA (57% exhibited>25% elevation, p=0.016).

With reference to FIG. 36B, the elevation in NETest (80-100% score) occurred at a significantly earlier time (94.5 days) than image-identifiable disease progression (241 days) in the 14 patients (*p<0.0001, Chi²=19). A similar analysis for CgA identified that this was not different to image-based assessment (FIG. 36B, 185.5 days vs. 241 days). CgA alterations occurred significantly later than the NETest (p=0.002, Chi²=13.6).

Utility of NETEST and Gene Expression for the Prediction of Disease Recurrence—Utility of NETEST To evaluate the utility of the NETest disease recurrence, the relationship between the NETest and clinically defined outcomes (per RECIST criteria) was evaluated in a long-term prospective study. Samples were collected both pre-therapy as well as at intervals up to five years in thirty four patients. Imaging was available to stage and categorize disease patterns pre- and during therapy (up to 65 months follow-up).

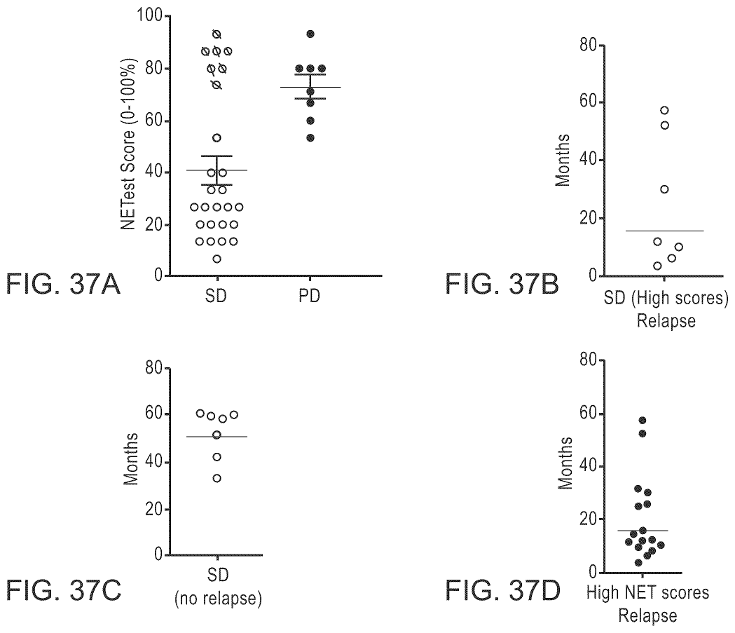

In this prospective sample set, the initial NETest scores were significantly elevated in the PD patients (median: 75%, range 53-94%) compared to the SD patients (median: 26%, range 7-94%; p=0.01) (FIG. 37A). Eight SD patients had levels>40%. Of these 7 developed disease recurrence in a median of 12.2 months (range 3.6-57.7; FIG. 37B). With reference to FIG. 37C, seven of the initial SD patients (with low NETest scores) did not develop recurrent disease. The median follow-up time was 58 months (range: 32-64).

Sixteen events of progressive disease were identified over the time course. Each was associated with elevated NETest (scores>80%). With reference to FIG. 37D, the median time to progression for patients with elevated scores was 13.4 months (range: 3.6-57).

Overall, 23/24 events where the NETest was elevated was associated with development of disease recurrence in median ˜13 months. Seven of seven with consistently low scores were disease free (up to 5 years). The accuracy of the test was 97%.

Utility of NET Genes as Surrogate Measure of Tumor Proliferation and Imaging—The utility of NETest genes as well as clusters of genes to function as surrogate markers of histopathological and imaging parameters was evaluated. A particular focus was placed on the Ki-67 index (a marker of tumor proliferation) and on somatostatin-based imaging e.g., ⁶⁸Ga-PET. This was undertaken to demonstrate that the NETest and elements thereof could have clinical utility as adjuncts for standard clinical measures. As an example, Ki-67 measurements are tissue based and therefore are invasive. Demonstrating a blood-derived correlate would provide a real-time measure of tumor growth without the need for a biopsy.

These analyses were conducted in two separate datasets: Dataset 1 (n=28) and Dataset 2 (n=22). Dataset 1 included patients who were collected for therapeutic intervention, namely peptide receptor radionucleotide therapy (PRRT). Dataset 2 included patients who exhibited stable disease and were undergoing routine follow-up.

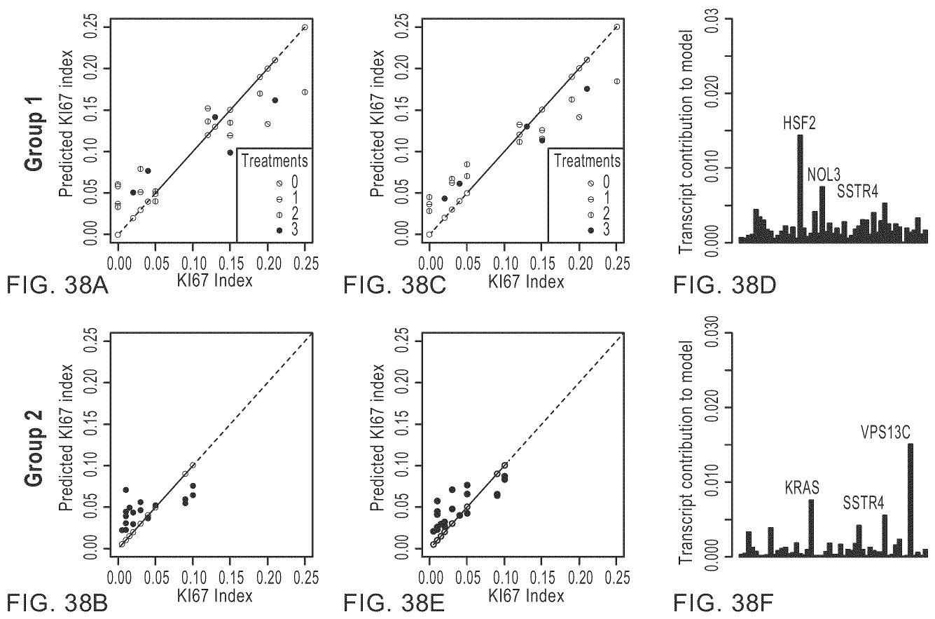

A Surrogate for the Ki-67 Index: Multivariate regression analysis did not identify any significant correlation between individual gene expression and the Ki-67 index (a marker of tumor proliferation) in either of the two groups. With reference to FIGS. 38A and 38B, examination of somatostatin receptor expression identified significant correlations (R=0.9, p=2×10⁻⁸) with Ki67 in each of the tumor groups.

An examination of all genes in the NETest identified significantly higher correlations with Ki-67 (R=0.93-98, p=10⁻⁹-10⁻¹³, FIGS. 38C-38E). The single most informative gene was SSTR4 (FIG. 38D-38F). These data demonstrate firstly, that the NETest as a whole can be used as a liquid biopsy to determine the proliferative index of the tumor i.e., provides a surrogate marker for a tissue-based histopathological measurement. Secondly, expression of circulating somatostatin receptor genes can also be used as a measure of tumor proliferation.

Proliferome+SSTRome algorithm is also referred to as Progressive Diagnostic V; the highly relevant genes (KRAS, SSTR4, and VPS13C) algorithm is also referred to as Progressive Diagnostic VI; the highly relevant genes+SSTRome algorithm is also referred to as Progressive Diagnostic VII.

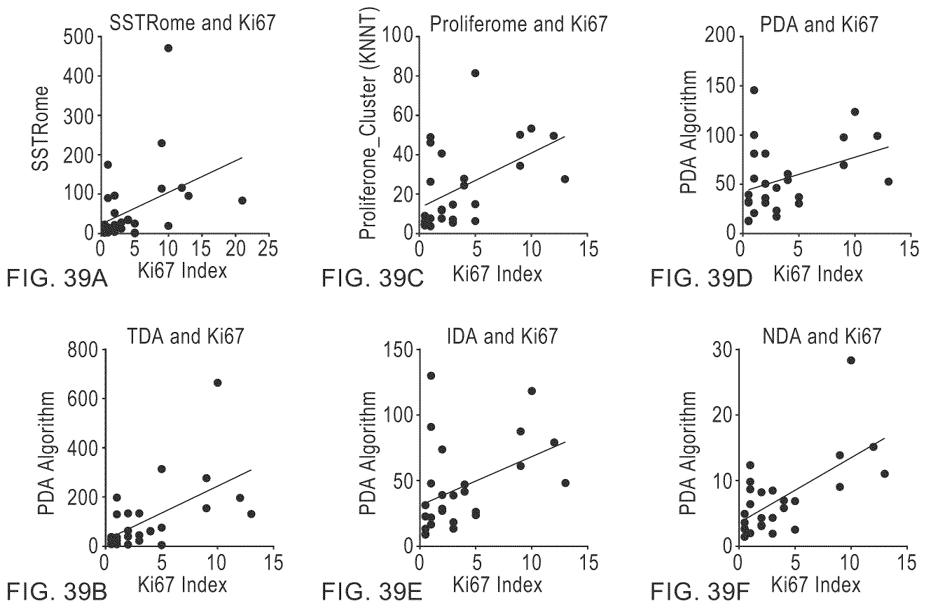

With reference to FIGS. 39A-39F, correlations (linear regression) between gene clusters (SSTRome and proliferome) or each of the algorithms and the Ki-67 index, are shown. Examination of individual gene clusters confirmed that the SSTRome and Proliferome correlated with the Ki-67 index (R=0.16-0.25, p<0.05, FIGS. 39A, 39C). Analysis of the algorithms identified that the NDA and TDA algorithms were highly correlated with the Ki-67 index (R=0.34-0.42, p<0.002, FIGS. 39B, 39F) while the PDA and IDA were less well-correlated (R=0.14-0.17, p=0.06, FIGS. 39D, 39E). These results demonstrate that gene clusters and algorithms including biologically relevant tumor information e.g., SSTRome can be utilized as a measure of tumor tissue proliferation.

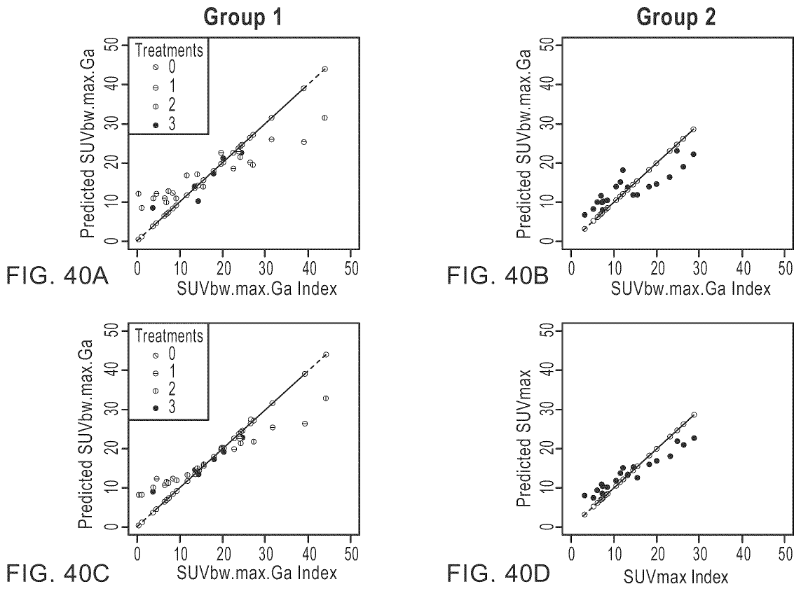

Relationship with Somatostatin-Based Imaging: Next was examined whether genes in the test correlated with two variables from somatostatin-based imaging, the SUVbmax (tumor uptake—a measure of receptor density/target availability) and the MTV (molecular tumor volume—a measure of the tumor burden). Multivariate regression analysis did not identify any single gene to correlate with the SUVmax. However, both the SSTRome as well as the NETest genes as a group were well correlated with the SUVmax. Correlations in both groups ranged between R=0.88-0.94 (p<10⁻⁷) for the SSTRome (FIGS. 40A-40B) and R=0.97-0.98, p<10⁻¹³ for the NET gene set (FIGS. 40C-40D).

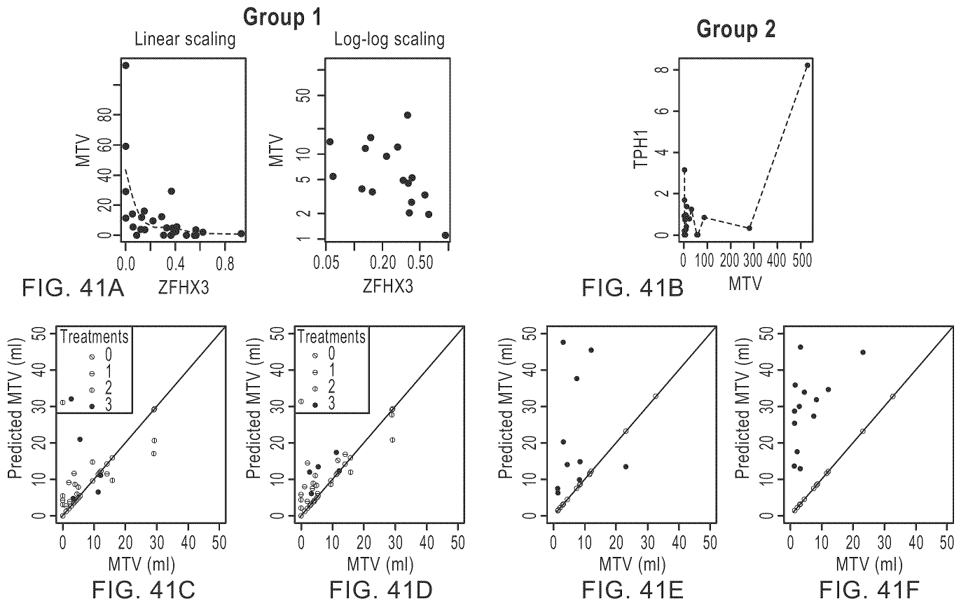

Multivariate regression analysis identified ZFHX3 as a marker of MTV in Group 1 (R=0.98, FIG. 41A) while TPH1 was correlated with MTV in Group 2 (R=0.76, FIG. 41B).

Similarly to the SUVmax, both the SSTRome as well as the NETest genes as a group were well correlated with the MTV. Correlations in both groups ranged between R=0.72-0.77 (p<10⁻⁴) for the SSTRome (FIGS. 41C-41E) and R=0.91-0.95, p<10⁻¹² for the NET gene set (FIGS. 41D-41F).

These data demonstrate that genes in the NETest correlate and can be used to estimate both the target availability for somatostatin analog-based therapies as well as provide a measure of the tumor burden. Both these aspects are critical for directing therapy as well as measuring the efficacy of therapy.

ZFHX3 as a Marker for Disease Assessment: The identification of ZFHX3 as the best marker for MTV, as shown in FIG. 41A, suggests that expression of this gene may have clinical utility as a measure of tumor burden and changes thereof. ZFHX3 is a zinc finger protein involved in the regulation of neuronal differentiation through the control of cell cycle arrest. Loss of ZFHX3 expression with a subsequent loss of cell cycle arrest therefore is related to tumor proliferation and the development of new lesions and/or progression of disease.

It was examined whether measurements of ZFHX3 may provide a marker of new growth/progression in NETs and if that alteration in ZFHX3 may reflect response to therapy or therapy failure (progression). Expression of this gene was initially assessed in patients who had evidence of new lesions.

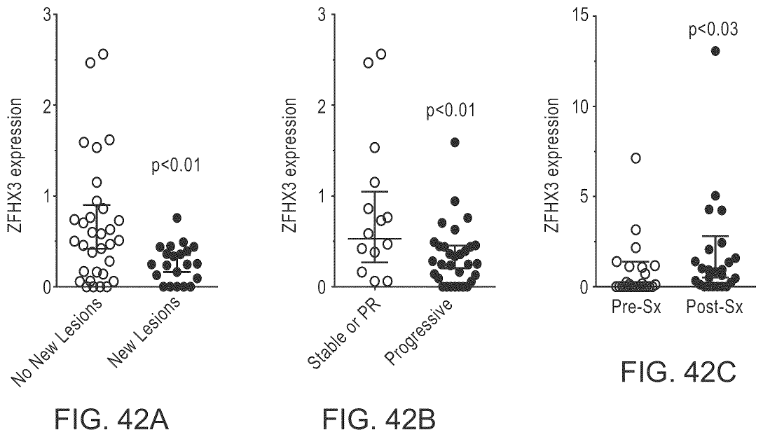

With reference to FIG. 42A, patients who had developed new lesions (identified by imaging) expressed significantly decreased ZFHX3. With reference to FIG. 42B, those patients that were determined as SD also have significantly higher levels than those who were progressive. Moreover, with reference to FIG. 42C, expression of the gene was increased following surgery.

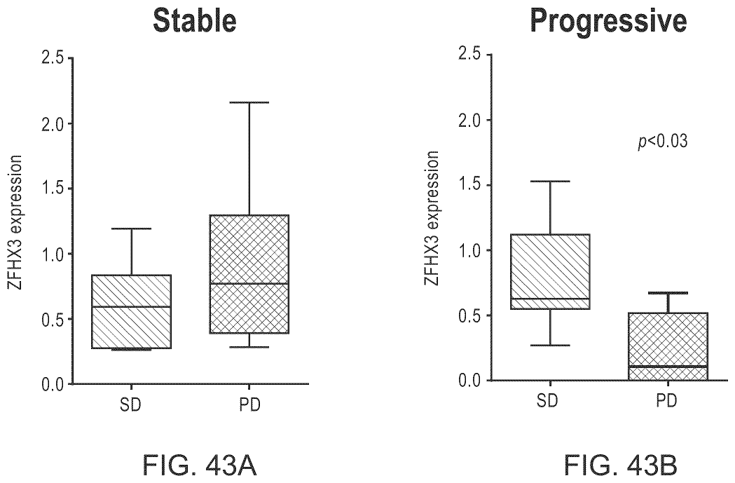

With reference to FIGS. 43A-43B, long-term follow-up (>3 years) in a group identified that patients who remained stable exhibited no changes in ZFHX3 expression over this time period, while patients who developed progressive disease had significantly lower expression levels.

These data demonstrate that ZFHX3 expression correlates with the development of new lesions and a decrease in expression can be used to define disease progression.

Utility of NETEST and Gene Expression for the Prediction of Therapeutic Efficacy—To further evaluate the utility of the NETest in therapy, the relationship between PRRT and clinically defined (per RECIST criteria) outcomes were evaluated. Samples were collected both pre-therapy as well as at follow-up in fifty-four patients. Imaging was available to stage and categorize disease patterns pre- and post-therapy (at 3 and 6 month follow-up).

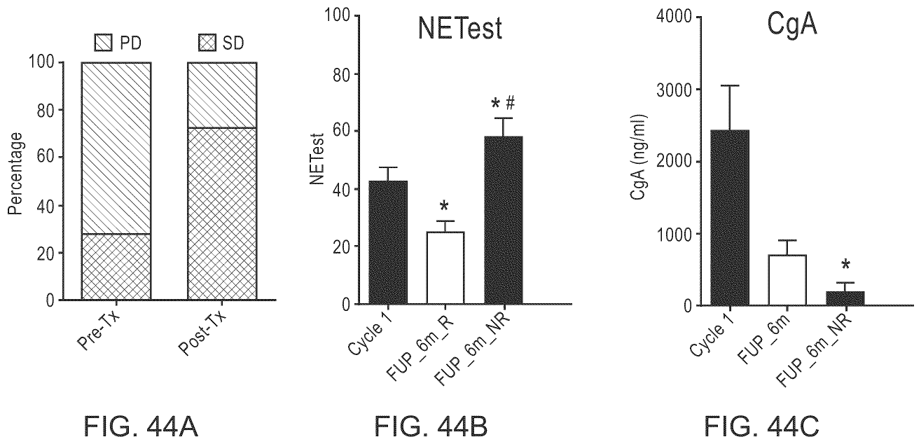

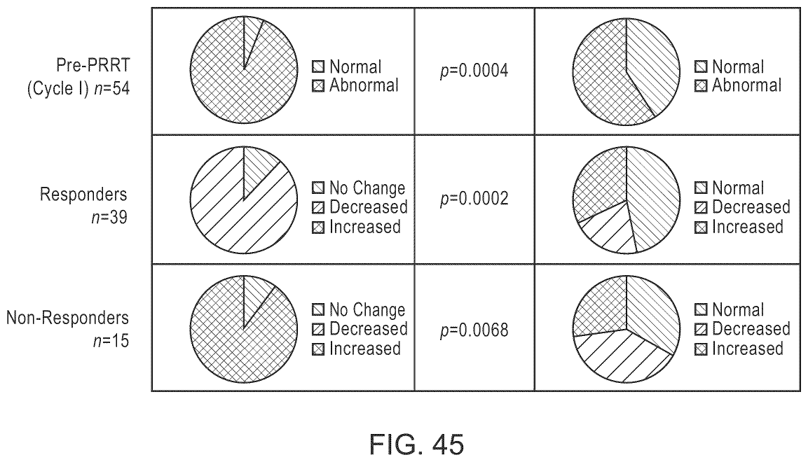

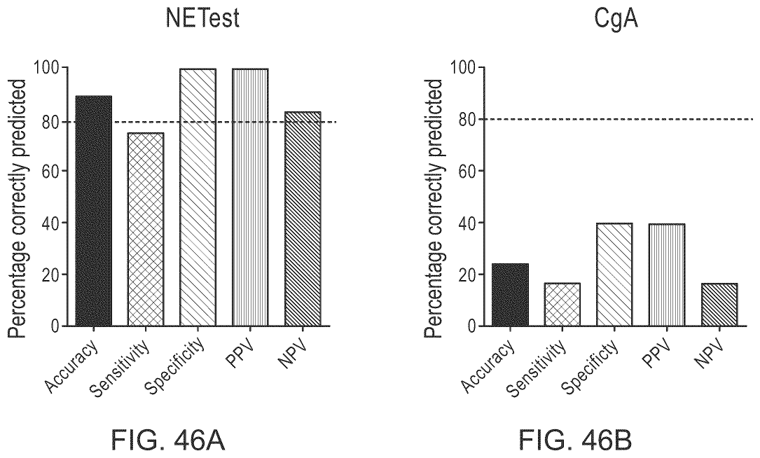

In this prospective sample set, radiotherapy significantly resulted in a reduction in the number of patients with progressive disease (FIG. 44A). Patients who did not respond to therapy i.e., categorized as progressive disease at the 6 month follow-up period exhibited an increase in the NETest score. The score was significantly reduced in patients with SD at this time point (FIG. 44B). No significant alterations were noted for CgA (FIG. 44C). Alterations in NETest paralleled changes in therapeutic responses (FIG. 45). The metrics for biomarkers and outcome identified that the NETest had an accuracy of 89%, sensitivity 75%, specificity 100%, PPV 100% and NPV 83% (FIG. 46A). With reference to FIG. 46B, CgA had an accuracy of 24%, sensitivity 17%, specificity 40%, PPV 40% and NPV 17%. The NETest significantly outperformed CgA (Chi-square=27.4; p=1.2×10⁻⁷).

Pre-treatment NETest scores as well as grading were available and used to identify whether a combination of gene expression and clinical parameters were predictive of outcome, i.e., response to therapy.

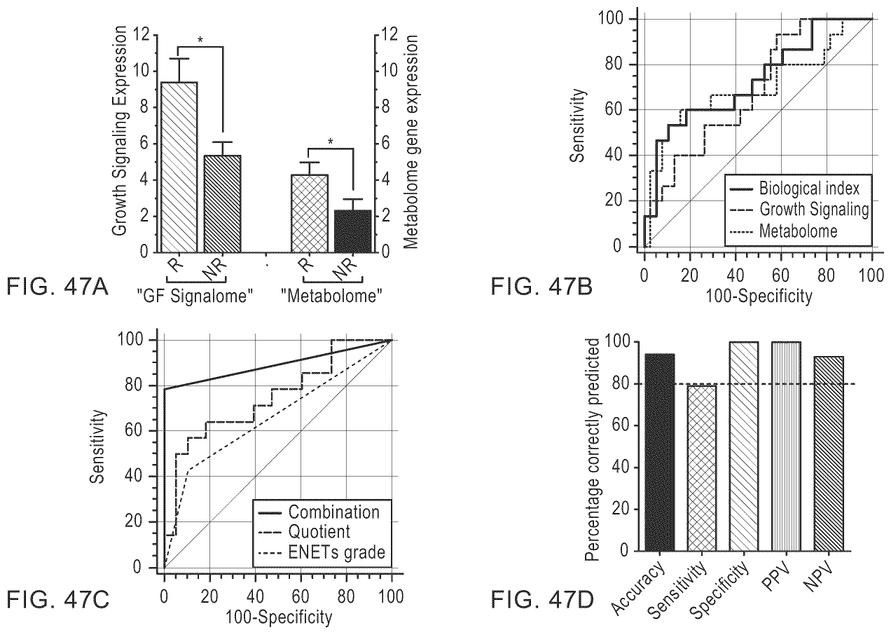

With reference to FIG. 47A, a subset of NETest gene expression levels were significantly different between responders and non-responders prior to therapy. These included genes linked to growth factor signaling (GF signalome: ARAF1, BRAF, KRAS and RAF1) as well as genes linked to metabolism (including ATP6V1H, OAZ2, PANK2, PLD) 3). Specifically, PRRT-responders exhibited significantly elevated growth factor signaling (9.4±1.3 vs. 5.3±0.7, p=0.05) and significantly elevated metabolomic gene expression (4.37 vs. 2.3±0.6, p=0.03) prior to PRRT. An integration of the two “clusters” (GF signalome+metabolome) into a “Biological Index” through summation of gene expression enabled prediction of future PRRT-responders from non-responders. A cut-off of 5.9 (normalized gene expression) exhibited >85% specificity for predicting response (>5.9 predicted PRRT responders) and resulted in an AUC of 0.74=0.08 (z-statistic=2.915, p=0.0036) (FIG. 47B).

No clinical parameters were predictive of PRRT response except tumor grade. Low grade tumors responded (77%) to therapy while ˜50% of high grade lesions were associated with responses. Grading alone was only 65% accurate (p=0.1). In contrast a “Prediction Quotient” which comprised the combination of the Biological Index (“GF signalome”+ “metabolome”) and the tumor grade was significantly (92%) more accurate. The Prediction Quotient had a significantly better AUC (0.90±0.07) than histological grade alone for predicting treatment response (AUC-0.66, difference between areas 0.23, z-statistic 2.25, p=0.024) (FIG. 47C).

The Prediction Quotient was also clinically useful. Patients could be segregated into Low Grade/High Ome and High Grade/Low Ome groups. The latter had a significantly lower PFS (17 months) than the low grade/high Ome group (PFS not reached, Log-rank: 26.8; p<0.0001: FIG. 47D). The Hazard Ratio was 53.3.

These results demonstrate that alterations in score correlate with treatment responses and that circulating NET transcript measurements prior to therapy are predictive of outcome to PRRT.

1.-38. (canceled)

39. A method for treating a progressive gastroenteropancreatic neuroendocrine neoplasm (GEP-NEN) in a human subject in need thereof, the method comprising:
determining the expression levels of at least 23 biomarkers from a test sample from the human subject by performing reverse transcription polymerase chain reaction (RT-PCR) with a plurality of probes or primers specific to detect the expression of the at least 23 biomarkers, wherein the at least 23 biomarkers comprise APLP2, ARAF, CD59, CTGF, FZD7, KRAS, MKI67/KI67, MORF4L2, NAPILI, NOL3, PNMA2, RAF1, RSF1, SLC18A2/VMAT2, SPATA7, SSTR1, SSTR3, SSTR4, SSTR5, TPH1, TRMT112, ZFHX3, and ALG9, wherein the test sample is blood, serum, plasma, or neoplastic tissue;
normalizing the expression levels of APLP2, ARAF, CD59, CTGF, FZD7, KRAS, MKI67/KI67, MORF4L2, NAPILI, NOL3, PNMA2, RAF1, RSF1, SLC18A2/VMAT2, SPATA7, SSTR1, SSTR3, SSTR4, SSTR5, TPH1, TRMT112, and ZFHX3 to the expression level of ALG9 to obtain normalized expression levels;
classifying the test sample with respect to the presence or development of a GEP-NEN using the normalized expression levels in a classification system, wherein the classification system is a machine learning system that comprises four different algorithms: Support Vector Machine, Linear Discrimination Analysis, K-Nearest Neighbor, and Naïve Bayes;
assigning a score based on a result of each of the four different algorithms;
comparing the score with a predetermined cutoff value;
determining the presence of a progressive GEP-NEN in the subject, wherein determining the presence of a progressive GEP-NEN in the subject comprises determining that the score is equal to or greater than the predetermined cutoff value, wherein the predetermined cutoff value is 5 on a MAARC-NET scoring system scale of 0-8;
administering a treatment to the subject identified as having a progressive GEP-NEN, wherein the treatment comprises surgery or drug therapy.

40. The method of claim 39, wherein determining the presence of a progressive GEP-NEN in the subject further comprises determining that the score is equal to or greater than a predetermined cutoff value of 5 and less than a predetermined cutoff value of 6;
determining the expression level of SMARCD3 from the test sample;
normalizing the expression level of SMARCD3 to the expression level of ALG9 to obtain a normalized expression level;
comparing the normalized expression level of SMARCD3 and TPH1 in the test sample with a first and a second predetermined cutoff value, respectively;
determining the presence of progressive GEP-NEN in the subject by determining that the normalized expression level of SMARCD3 is equal to or less than the first predetermined cutoff value and the expression level of TPH1 is less than the second predetermined cutoff value.

41. The method of claim 39, wherein determining the presence of a progressive GEP-NEN in the subject further comprises determining that the score is equal to or greater than a predetermined cutoff value of 6 and less than a predetermined cutoff value of 7;
determining the expression levels of VMAT1 and PHF21A from the test sample;
normalizing the expression levels of VMAT1 and PHF21A to the expression level of ALG9;
comparing the normalized expression level of VMAT1 and PHF21A with a first and a second predetermined cutoff value, respectively; and
determining the presence of progressive GEP-NEN in the subject by determining that the normalized expression level of VMAT1 is equal to or greater than the first predetermined cutoff value and the expression level of PHF21A is equal to or greater than the second predetermined cutoff value.

42. The method of claim 39, wherein determining the presence of a progressive GEP-NEN in the subject further comprises determining that the score is equal to or greater than a predetermined cutoff value of 7 and less than a predetermined cutoff value of 8, according to the method of claim 1;
determining the expression levels of VMAT1 and PHF21A from the test sample,
normalizing the expression levels of VMAT1 and PHF21A to the expression level of ALG9;
comparing the normalized expression level of VMAT1 and PHF21A with a first and a second predetermined cutoff value, respectively; and
determining the presence of progressive GEP-NEN in the subject by determining that the normalized expression level of VMAT1 is equal to or greater than the first predetermined cutoff value and the expression level of PHF21A is equal to or less than the second predetermined cutoff value.

43. The method of claim 39, wherein determining the presence of a progressive GEP-NEN in the subject further comprises determining that the score is equal to a predetermined cutoff value of 8, according to the method of claim 1;
determining the expression level of ZZZ3 from the test sample;
normalizing the expression level of ZZZ3 to the expression level of ALG9;
comparing the normalized expression level of ZZZ3 with a predetermined cutoff value; and
determining the presence of progressive GEP-NEN in the subject by determining that the normalized expression level of ZZZ3 is equal to or less than the predetermined cutoff value.

44. The method of claim 39, wherein the biomarker is RNA or cDNA.

45. The method of claim 44, wherein when the biomarker is RNA, the RNA is reverse transcribed to produce cDNA, and the produced cDNA expression level is detected.

46. The method of claim 39, wherein the expression level of the biomarker is detected by forming a complex between the biomarker and a labeled probe or primer.

47. The method of claim 44, wherein when the biomarker is RNA or cDNA, the RNA or cDNA detected by forming a complex between the RNA or cDNA and a labeled nucleic acid probe or primer.

48. The method of claim 47, wherein when the label is a fluorescent label.

49. The method of claim 47, wherein the complex between the RNA or cDNA and the labeled nucleic acid probe or primer is a hybridization complex.

50. The method of claim 39, wherein a subject in need thereof is a subject diagnosed with a GEP-NEN, a subject having at least one GEP-NEN symptom or a subject having a predisposition or familial history for developing a GEP-NEN.

51. The method of claim 39, wherein the drug therapy comprises somatostatin analog treatment, peptide receptor radionuclide therapy (PRRT) or any combination thereof.

52. The method of claim 39, wherein the drug therapy comprises somatostatin analog treatment.

53. The method of claim 39, wherein the drug therapy comprises peptide receptor radionuclide therapy (PRRT).

54. A method for determining a response of a peptide receptor radionucleotide therapy (PRRT) of a GEP-NEN in a subject in need thereof, comprising:
determining the expression level of each of 8 biomarkers from a test sample from the subject and a reference sample by contacting the test sample and the reference sample with a plurality of agents specific to detect the expression of each of the 8 biomarkers, wherein the 8 biomarkers comprise ARAF1, BRAF, KRAS, RAF1, ATP6V1H, OAZ2, PANK2, PLD3;
normalizing the expression level of the 8 biomarkers in the test sample to the expression level of the 8 biomarkers in the reference sample;
comparing the normalized expression level of the 8 biomarkers in the test sample with a predetermined cutoff value;
determining the presence of a PRRT-responsive GEP-NEN in the subject
when the normalized expression level of the 8 biomarkers is greater than a predetermined cutoff value.

55. A method for determining a response of a peptide receptor radionucleotide therapy (PRRT) of a GEP-NEN in a subject in need thereof, comprising:
(a) following a first cycle of PRRT therapy:
determining the expression level of at least 22 biomarkers from a first cycle test sample from the subject by contacting the first cycle test sample with a plurality of agents specific to detect the expression of the at least 22 biomarkers, wherein the 22 biomarkers are selected from the group consisting of APLP2, ARAF, ATP6V1H, BNIP3L, BRAF, CD59, COMMD9, CTGF, FZD7, GLT8D1, KRAS, MKI67/KI67, MORF4L2, NAP1L1, NOL3, OAZ2, PANK2, PHF21A, PLD3, PNMA2, PQBP1, RAF1, RNF41, RSF1, SLC18A1/VMAT1, SLC18A2/VMAT2, SMARCD3, SPATA7, SSTR1, SSTR3, SSTR4, SSTR5, TECPR2, TPH1, TRMT112, WDFY3, ZFHX3 and ZZZ3;
determining the expression level of the at least 22 biomarkers from a reference sample by contacting the reference sample with a plurality of agents specific to detect the expression of the at least 22 biomarkers;
normalizing the expression level of the at least 22 biomarkers in the first cycle test sample to the expression level of the at least 22 biomarkers in the reference sample;
(b) following a second cycle of PRRT therapy:
determining the expression level of at least 22 biomarkers from a second cycle test sample from the subject by contacting the test sample with a plurality of agents specific to detect the expression of the at least 22 biomarkers, wherein the 22 biomarkers are selected from the group consisting of APLP2, ARAF, ATP6V1H, BNIP3L, BRAF, CD59, COMMD9, CTGF, FZD7, GLT8D1, KRAS, MKI67/KI67, MORF4L2, NAP1L1, NOL3, OAZ2, PANK2, PHF21A, PLD3, PNMA2, PQBP1, RAF1, RNF41, RSF1, SLC18A1/VMAT1, SLC18A2/VMAT2, SMARCD3, SPATA7, SSTR1, SSTR3, SSTR4, SSTR5, TECPR2, TPH1, TRMT112, WDFY3, ZFHX3 and ZZZ3;
determining the expression level of the at least 22 biomarkers from a reference sample by contacting the reference sample with a plurality of agents specific to detect the expression of the at least 22 biomarkers;
normalizing the expression level of the at least 22 biomarkers in the second cycle test sample to the expression level of the at least 22 biomarkers in the reference sample;
(c) determining a ratio of change of the normalized expression levels from (a) to the normalized expression levels from (b);
(d) determining the presence of a PRRT-responsive GEP-NEN when the ratio of change is greater than a pre-PRRT therapy cutoff value.


SEQUENCE LISTING (52 sequences)

SEQ ID NO: 1 - DNA, 6158 bp, Homo sapiens
  source 1..6158: mol_type = other DNA
gtcttttgtccctcggcggacaccgtttgccagccaaagctatgtctgcgcgctcaccgacttcatagggtgccgaattcttttttccccaggcttgccatggctagtcgaggggctcggcagcgcctgaagggcagcggggccagcagtggggatacggccccggctgcggacaagctgcgggagctgctgggcagccgagaggcgggcggcgcggagcaccggaccgagttatctgggaacaaagcaggacaagtctgggcacctgaaggatctactgctttcaagtgtctgctttcagcaaggttatgtgctgctctcctgagcaacatctctgactgtgatgaaacattcaactactgggagccaacacactacctcatctatggggaagggtttcagacttgggaatattccccagcatatgccattcgctcctatgcttacctgttgcttcatgcctggccagctgcatttcatgcaagaattctacaaactaataagattcttgtgttttactttttgcgatgtcttctggcttttgtgagctgtatttgtgaactttacttttacaaggctgtgtgcaagaagtttgggttgcacgtgagtcgaatgatgctagccttcttggttctcagcactggcatgttttgctcatcatcagcattccttcctagtagcttctgtatgtacactacgttgatagccatgactggatggtatatggacaagacttccattgctgtgctgggagtagcagctggggctatcttaggctggccattcagtgcagctcttggtttacccattgcctttgatttgctggtcatgaaacacaggtggaagagtttctttcattggtcgctgatggccctcatactatttctggtgcctgtggtggtcattgacagctactattatgggaagttggtgattgcaccactcaacattgttttgtataatgtctttactcctcatggacctgatctttatggtacagaaccctggtatttctatttaattaatggatttctgaatttcaatgtagcctttgctttggctctcctagtcctaccactgacttctcttatggaatacctgctgcagagatttcatgttcagaatttaggccacccgtattggcttaccttggctccaatgtatatttggtttataattttcttcatccagcctcacaaagaggagagatttcttttccctgtgtatccacttatatgtctctgtggcgctgtggctctctctgcacttcagcacagttttctgtacttccagaaatgttaccactttgtgtttcaacgatatcgcctggagcactatactgtgacatcgaattggctggcattaggaactgtcttcctgtttgggctcttgtcattttctcgctctgtggcactgttcagaggatatcacgggccccttgatttgtatccagaattttaccgaattgctacagacccaaccatccacactgtcccagaaggcagacctgtgaatgtctgtgtgggaaaagagtggtatcgatttcccagcagcttccttcttcctgacaattggcagcttcagttcattccatcagagttcagaggtcagttaccaaaaccttttgcagaaggacctctggccacccggattgttcctactgacatgaatgaccagaatctagaagagccatccagatatattgatatcagtaaatgccattatttagtggatttggacaccatgagagaaacaccccgggagccaaaatattcatccaataaagaagaatggatcagcttggcctatagaccattccttgatgcttctagatcttcaaagctgctgcgggcattctatgtccccttcctgtcagatcagtatacagtgtacgtaaactacaccatcctcaaaccccggaaagcaaagcaaatcaggaagaaaagtggaggttagcaacacacctgtggccccaaaggacaaccatcttgttaactattgattccagtgacctgactccctgcaagtcatcgcctgtaacatttgtaataaaggtcttctgacatgaatactggaatctgggtgctctgggctagtcaaagtctatttcaaagtctaatcaaagtcacatttgctccctgtgtgtgtctctgttctgcatgtaaactttttgcagctaggcagagaaaggccctaaagcacagatagatatattgctccacatctcattgtttttcctctgttcaattatttactagaccggagaagagcagaaccaacttacaggaagaattgaaaatcctggtactggatggctgtgataagctgttctccacactctggcctggcatctgagaactagcaagcctctcttaggccatatgggcttctccaccaaagctgtttggcagctcctagcagaccttcttattgaaatcctcatgctgaaaatgaacacagcctagttgccaacccacatgtccttttcacctccagcaagactaagcttctttaaagcacttcacaggactaggaccctgtcctggagctatctcaggaaaaaggtgaccatttgaggaactgtgacctaattttattataatgatgcctctaattttcatttcctttacaaccaactgtaactataaggttgtattgcttttttgttcagttttagcatgctattttttgaattctagactcctccatgtgaagatatcaacagacaaaactacaactgtataggacatatttggagaaaattctatcaattgatacatttggatgacatcacatttttaagtaatgtaatctgaggccattgctgaggaaattaagaattttcctttttttttaaccacccccagtgaaaaggatcagtgtatatttatagcacctattttttagttctgtctgttgtgaggcacatcctgcatggggcacttctagtcaaataggcaatgataaggacctaattaaaatgtgataagtgtatactattactttaaaagcctttacagtcagtacttcagtttacaaggcactttcacagcatctcgtttgatcctcacagtcacaacatgtggtagacaaggcaggtgatttttatccccattttacagataaggaaacaggctgcgggtggggagtgaggggaggtaaagatagttagttgcctaaggtcacacagccagtaagtaatagagctgggactggaacccaggtttccttactctcatctattgctcctccatattcctcactcaaccatgaaaacattacttgaaaggactgatgaggttaaccagagacctaactgatattgtaactttctattttaaggaagaattgtgtctgtatttgagttctttggagcctccagtctgcctgtgtgttagaccagcacagcagtgctgtgtgatgcagcctgacctgtggcaggaaagtagtgcttctgtttggaagtcatgttcttttgcagccacacaggatccaaatatcagtactattcctgtagtcaatctggggtcacattataggtgccttatttccctaagggtaactgatctgaatatctgcaaataggatgaatctatttttcagaagttccatctttcatttttctttttttttttgagacagagtctcattctgtcgcccatgctggagtgcagtggcgcgatctcggctcgctgcaacctctgcctcccaggttgaagcaattctcatgcctcagccacccgagtagctgggattacaggcatgcgccatcatgcccagctaatttatgtatttttagtagagttggagtttcaccatgttggccaggctggtcttggactcctgacctcaggtcatccacccgcctcagcctcccaaagtgctggtattacaggcgtgagccaccgcacccagccccatctttcattttcaaagagaagggcattctaataggaactggtgccaagagagaagaaaagaagtgataacagaagaaatggctagttacaatattaaaaagctcctctttgagatctcctctgcaggaatatcagagacggagttgaagcgctggagaggtaataggtctagacagtacagaacaataactggggagtgtgtgaggatagactgggctcccccttgcttgaaagatctctggcatttaattctcaattcttgattactattttccagtgtaaaactagcacatatgatctgactacaggacagagaattttaagtgaaacatttgccttacttgcagtaataatgtgctgttcttcacagtagctaaggccctctatgtttcccagaggtaaataagaatccaggaatggaggtccatctgtgatgaatggcttttttctaatcaaagtagtataatgctgttttatctgttttgtcatcttgtttttttttttttttaaaaaaacaaaaccttaattataatatagcgcaaagaaaggccaggactgatgcagggattccttggaaatatcagttcctatcacttttaaaacctgattttggatctctctgttctatgtatgtctttagtgagagcacaatacatggcagaacgctgtgccaaatgttataggtaaggaatatagaaatgaatgttttttgttgtgaaggtgttttcatgtgatattttataaacacattttaaaaaatctccatcactttttagtataggaaggatagctttgcctgggaaaaacagtttcaacacacctgctcagagtagcagttctccctcaaaaaagcagtgttcagcctgcactgactgttctgcttgccaaaaggaggaagcatgcaagatacttatttctccatagattgtggagtatagagggatgtgggactacagattattattttttttccccgagacagagtcttgctctgtcgcccaggttggaacacaatggcacgacctcagctcactgcaacctctgtctcccgggttcaagcaattctcctgcttcagcctcctgagtagctgggattacaggcacacaccaccaccgcactcagctaatttttgtatttttagtagaggtggggttttaccatgttggccaggctggtcttaaactcctgaccttgtaatcatcccgcctcggcctcctaaagtgctaggattacaggcatgagccaccgcacccggcccagataatttttaatagcctttgatcatggggtgagtgagggagtaggtatacttggcaaatgcatggttctctgatttctagctctaaagcagccttatctgaatccccaaatcttgtgatgctgagtaccattactgaaccagtctgcacggtaggcatctgctaccaaaatttacctcctacctggtaggtgtcatctgataagaaagaagacaggttattttaattttttgagataatcacagaaaattgcagcccatactctttattaccgaattcaagtttggaaatagaccctttgttttaaatcatgatgggtctttatcccaatcatttatctgggtcatttttccaactttggagttctaggaaagaaccttgaaaacctgatatgattctgcagcatgaggtctacggtgaccatttgggcaaagctccagtggcaatcatttattgtgttttgcatttcctgggatttattgaaataagaattcactgtgattatgtagtcttctggctagtatcaggcagctctgcttttaatttggttaattttattttctctgaagagggagaagaggtacaatttaatcttggcctccacaagcatattaaagctcacgtgttaatcagtgcattcttatgctcctacattaaatgccttgggtaaatggataaatggacatgtgcccagctttaattttttttgcaacagaaagatcagacttccgtatggcatcgttggatttcagaggctttctggtgtatctgtaaatctgaatgttgccttctgccagtctgtataaccaggtgattcatgctgcaaatgaaatcaggaagcagtaaagtgttaaagcaagagtattgtccaattcacttgtcttcctgatccttgtactttatttcacgtgtcggtgtttacattacatacttatatttcctgtgaaagaaagagttaaataaattgtagcagtttga

SEQ ID NO: 2 - DNA, 2231 bp, Homo sapiens
  source 1..2231: mol_type = other DNA
actgatatgaggaggcatagagatagacagcggttccttccaatagacgtgaagccgaggccggtatgagccaatgcggtcgggaggcggggctcgggtgtgtgtggaggggaccctgtggttagcagcagctatcgcagcgtcggatgttcagagcagcagaagccggcgtcgtcggatgttgtgttgcccgccaccatgagctacacaggctttgtccagggatctgaaaccactttgcagtcgacatactcggataccagcgctcagcccacctgtgattatggatatggaacttggaactctgggacaaatagaggctacgagggctatggctatggctatggctatggccaggataacaccaccaactatgggtatggtatggccacttcacactcttgggaaatgcctagctctgacacaaatgcaaacactagtgcctcgggtagcgccagtgccgattccgttttatccagaattaaccagcgcttagatatggtgccgcatttggagacagacatgatgcaaggaggcgtgtacggctcaggtggagaaaggtatgactcttatgagtcctgcgactcgagggccgtcctgagtgagcgcgacctgtaccggtcaggctatgactacagcgagcttgaccctgagatggaaatggcctatgagggccaatacgatgcctaccgcgaccagttccgcatgcgtggcaacgacaccttcggtcccagggcacagggctgggcccgggatgcccggagcggccggccaatggcctcaggctatgggcgcatgtgggaagaccccatgggggcccggggccagtgcatgtctggtgcctctcggctgccctccctcttctcccagaacatcatccccgagtacggcatgttccagggcatgcgaggtgggggcgccttcccgggcggctcccgctttggtttcgggtttggcaatggcatgaagcagatgaggcggacctggaagacctggaccacagccgacttccgaaccaagaagaagaagagaaagcagggcggcagtcctgatgagccagatagcaaagccacccgcacggactgctcggacaacagcgactcagacaatgatgagggcaccgagggggaagccacagagggccttgaaggcaccgaggctgtggagaagggctccagagtggacggagaggatgaggagggaaaagaggatgggagagaagaaggcaaagaggatccagagaagggggccctaaccacccaggatgaaaatggccagaccaagcgcaagttgcaggcaggcaagaagagtcaggacaagcagaaaaagcggcagcgagaccgcatggtggaaaggatccagtttgtgtgttctctgtgcaaataccggaccttctatgaggacgagatggccagccatcttgacagcaagttccacaaggaacactttaagtacgtaggcaccaagctccctaagcagacggctgactttctgcaggagtacgtcactaacaagaccaagaagacagaggagctccgaaaaaccgtggaggaccttgatggcctcatccagcaaatctacagagaccaggatctgacccaggaaattgccatggagcattttgtgaagaaggtggaggcagcccattgtgcagcctgcgacctcttcattcccatgcagtttgggatcatccagaagcatctgaagaccatggatcacaaccggaaccgcaggctcatgatggagcagtccaagaagtcctccctcatggtggcccgcagtattctcaacaacaagctcatcagcaagaagctggagcgctacctgaagggcgagaaccctttcaccgacagccccgaggaggagaaggagcaggaggaggctgagggcggtgccctggacgagggggcgcagggcgaagcggcagggatctcggagggcgcagagggcgtgccggcgcagcctcccgtgcccccagagccagcccccggggccgtgtcgccgccaccgccgccgcccccagaggaggaggaggagggcgccgtgcccttgctgggaggggcgctgcaacgccagatccgcggcatcccgggcctcgacgtggaggacgacgaggagggcggcgggggcgccccgtgacccgagctcggggcgggcggagcccgcgtggccgaagctggaaaccaaacctaataaagttttcccatcccaccaaaaaaaaaaaaaaaaaa

SEQ ID NO: 3 - DNA, 3791 bp, Homo sapiens
  source 1..3791: mol_type = other DNA
agaaggagggcgtggtaatatgaagtcagttccggttggtgtaaaacccccggggcggcggcgaactggctttagatgcttctgggtcgcggtgtgctaagcgaggagtccgagtgtgtgagcttgagagccgcgcgctagagcgacccggcgagggatggcggccaccgggaccgcggccgccgcagccacgggcaggctcctgcttctgctgctggtggggctcacggcgcctgccttggcgctggccggctacatcgaggctcttgcagccaatgccggaacaggatttgctgttgctgagcctcaaatcgcaatgttttgtgggaagttaaatatgcatgtgaacattcagactgggaaatgggaacctgatccaacaggcaccaagagctgctttgaaacaaaagaagaagttcttcagtactgtcaggagatgtatccagagctacagatcacaaatgtgatggaggcaaaccagcgggttagtattgacaactggtgccggagggacaaaaagcaatgcaagagtcgctttgttacacctttcaagtgtctcgtgggtgaatttgtaagtgatgtcctgctagttccagaaaagtgccagtttttccacaaagagcggatggaggtgtgtgagaatcaccagcactggcacacggtagtcaaagaggcatgtctgactcagggaatgaccttatatagctacggcatgctgctcccatgtggggtagaccagttccatggcactgaatatgtgtgctgccctcagacaaagattattggatctgtgtcaaaagaagaggaagaggaagatgaagaggaagaggaagaggaagatgaagaggaagactatgatgtttataaaagtgaatttcctactgaagcagatctggaagacttcacagaagcagctgtggatgaggatgatgaggatgaggaagaaggggaggaagtggtggaggaccgagattactactatgacaccttcaaaggagatgactacaatgaggagaatcctactgaacccggcagcgacggcaccatgtcagacaaggaaattactcatgatgtcaaagctgtctgctcccaggaggcgatgacggggccctgccgggccgtgatgcctcgttggtacttcgacctctccaagggaaagtgcgtgcgctttatatatggtggctgcggcggcaacaggaacaattttgagtctgaggattattgtatggctgtgtgtaaagcgatgattcctccaactcctctgccaaccaatgatgttgatgtgtatttcgagacctctgcagatgataatgagcatgctcgcttccagaaggctaaggagcagctggagattcggcaccgcaaccgaatggacagggtaaagaaggaatgggaagaggcagagcttcaagctaagaacctccccaaagcagagaggcagactctgattcagcacttccaagccatggttaaagctttagagaaggaagcagccagtgagaagcagcagctggtggagacccacctggcccgagtggaagctatgctgaatgaccgccgtcggatggctctggagaactacctggctgccttgcagtctgacccgccacggcctcatcgcattctccaggccttacggcgttatgtccgtgctgagaacaaagatcgcttacataccatccgtcattaccagcatgtgttggctgttgacccagaaaaggcggcccagatgaaatcccaggtgatgacacatctccacgtgattgaagaaaggaggaaccaaagcctctctctgctctacaaagtaccttatgtagcccaagaaattcaagaggaaattgatgagctccttcaggagcagcgtgcagatatggaccagttcactgcctcaatctcagagacccctgtggacgtccgggtgagctctgaggagagtgaggagatcccaccgttccaccccttccaccccttcccagccctacctgagaacgaaggatctggagtgggagagcaggatgggggactgatcggtgccgaagagaaagtgattaacagtaagaataaagtggatgaaaacatggtcattgacgagactctggatgttaaggaaatgattttcaatgccgagagagttggaggcctcgaggaagagcgggaatccgtgggcccactgcgggaggacttcagtctgagtagcagtgctctcattggcctgctggtcatcgcagtggccattgccacggtcatcgtcatcagcctggtgatgctgaggaagaggcagtatggcaccatcagccacgggatcgtggaggttgatccaatgctcaccccagaagagcgtcacctgaacaagatgcagaaccatggctatgagaaccccacctacaaatacctggagcagatgcagatttaggtggcagggagcgcggcagccctggcggagggatgcaggtgggccggaagatcccacgattccgatcgactgccaagcagcagccgctgccaggggctgcgtctgacatcctgacctcctggactgtaggactatataaagtactactgtagaactgcaatttccattcttttaaatgggtgaaaaatggtaatataacaatatatgatatataaaccttaaatgaaaaaaatgatctattgcagatatttgatgtagttttcttttttaaattaatcagaaaccccacttccattgtattgtctgacacatgctctcaatatataataaatgggaaatgtcgattttcaataatagacttatatgcaggctgtcgttccggttatgttgtgtaagtcaactcttcagcctcattcactgtcctggcttttatttaaagaaaaaaaaggcagtattccctttttaaatgagctttcaggaagttgctgagaaatggggtggaatagggaactgtaatggccactgaagcacgtgagagaccctcgcaaaatgatgtgaaaggaccagtttcttgaagtccagtgtttccacggctggatacctgtgtgtctccataaaagtcctgtcaccaaggacgttaaaggcattttattccagcgtcttctagagagcttagtgtatacagatgagggtgtccgctgctgctttccttcggaatccagtgcttccacagagattagcctgtagcttatatttgacattcttcactgtctgttgtttacctaccgtagctttttaccgttcacttccccttccaactatgtccagatgtgcaggctcctcctctctggactttctccaaaggcactgaccctcggcctctactttgtcccctcacctccaccccctcctgtcaccggccttgtgacattcactcagagaagaccacaccaaggaggcggccgctggcccaggagagaacacggggaggtttgtttgtgtgaaaggaaagtagtccaggctgtccctgaaactgagtctgtggacactgtggaaagctttgaacaattgtgttttcgtcacaggagtctttgtaatgcttgtacagttgatgtcgatgctcactgcttctgctttttctttctttttattttaaatctgaaggttctggtaacctgtggtgtatttttattttcctgtgactgtttttgttttgtttttttcctttttcctcccctttgaccctattcatgtctctacccactatgcacagattaaacttcacctacaaactccttaatatgatctgtggagaatgtacacagtttaaacacatcaataaatactttaacttccaccgagaaaaaaaaaaaaaaaa

SEQ ID NO: 4 - DNA, 2562 bp, Homo sapiens
  source 1..2562: mol_type = other DNA
cttgacagacgtgaccctgacccaataagggtggaaggctgagtcccgcagagccaataacgagagtccgagaggcgacggaggcggactctgtgaggaaacaagaagagaggcccaagatggagacggcggcggctgtagcggcgtgacaggagccccatggcacctgcccagccccacctcagcccatcttgacaaaatctaaggctccatggagccaccacggggcccccctgccaatggggccgagccatcccgggcagtgggcaccgtcaaagtatacctgcccaacaagcaacgcacggtggtgactgtccgggatggcatgagtgtctacgactctctagacaaggccctgaaggtgcggggtctaaatcaggactgctgtgtggtctaccgactcatcaagggacgaaagacggtcactgcctgggacacagccattgctcccctggatggcgaggagctcattgtcgaggtccttgaagatgtcccgctgaccatgcacaattttgtacggaagaccttcttcagcctggcgttctgtgacttctgccttaagtttctgttccatggcttccgttgccaaacctgtggctacaagttccaccagcattgttcctccaaggtccccacagtctgtgttgacatgagtaccaaccgccaacagttctaccacagtgtccaggatttgtccggaggctccagacagcatgaggctccctcgaaccgccccctgaatgagttgctaaccccccagggtcccagcccccgcacccagcactgtgacccggagcacttccccttccctgccccagccaatgcccccctacagcgcatccgctccacgtccactcccaacgtccatatggtcagcaccacggcccccatggactccaacctcatccagctcactggccagagtttcagcactgatgctgccggtagtagaggaggtagtgatggaaccccccgggggagccccagcccagccagcgtgtcctcggggaggaagtccccacattccaagtcaccagcagagcagcgcgagcggaagtccttggccgatgacaagaagaaagtgaagaacctggggtaccgggactcaggctattactgggaggtaccacccagtgaggtgcagctgctgaagaggatcgggacgggctcgtttggcaccgtgtttcgagggcggtggcatggcgatgtggccgtgaaggtgctcaaggtgtcccagcccacagctgagcaggcccaggctttcaagaatgagatgcaggtgctcaggaagacgcgacatgtcaacatcttgctgtttatgggcttcatgacccggccgggatttgccatcatcacacagtggtgtgagggctccagcctctaccatcacctgcatgtggccgacacacgcttcgacatggtccagctcatcgacgtggcccggcagactgcccagggcatggactacctccatgccaagaacatcatccaccgagatctcaagtctaacaacatcttcctacatgaggggctcacggtgaagatcggtgactttggcttggccacagtgaagactcgatggagcggggcccagcccttggagcagccctcaggatctgtgctgtggatggcagctgaggtgatccgtatgcaggacccgaacccctacagcttccagtcagacgtctatgcctacggggttgtgctctacgagcttatgactggctcactgccttacagccacattggctgccgtgaccagattatctttatggtgggccgtggctatctgtccccggacctcagcaaaatctccagcaactgccccaaggccatgcggcgcctgctgtctgactgcctcaagttccagcgggaggagcggcccctcttcccccagatcctggccacaattgagctgctgcaacggtcactccccaagattgagcggagtgcctcggaaccctccttgcaccgcacccaggccgatgagttgcctgcctgcctactcagcgcagcccgccttgtgccttaggccccgcccaagccaccagggagccaatctcagccctccacgccaaggagccttgcccaccagccaatcaatgttcgtctctgccctgatgctgcctcaggatcccccattccccaccctgggagatgagggggtccccatgtgcttttccagttcttctggaattgggggacccccgccaaagactgagccccctgtctcctccatcatttggtttcctcttggctttggggatacttctaaattttgggagctcctccatctccaatggctgggatttgtggcagggattccactcagaacctctctggaatttgtgcctgatgtgccttccactggattttggggttcccagcaccccatgtggattttggggggtcccttttgtgtctcccccgccattcaaggactcctctctttcttcaccaagaagcacagaattctgctgggcctttgcttgtttaaaaaaaaaaaaaaaaaaaaaaaaaaaaaaaaaaaaaaaaaaaaaaaaaaaaaaaaaaaaaaaaaaa

SEQ ID NO: 5 - DNA, 2186 bp, Homo sapiens
  source 1..2186: mol_type = other DNA
agcagtcacgtgcctccgatcacgtgaccggcgcctctgtcattctactgcggccgccctggcttccttctacctgtgcggccctcaacgtctccttggtgcgggacccgcttcactttcggctcccggagtctccctccactgctcagacctctggacctgacaggagacgcctacttggctctgacgcggcgccccagcccggctgtgtccccggcgccccggaccaccctccctgccggctttgggtgcgttgtggggtcccgaggattcgcgagatttgttgaaagacattcaagattacgaagtttagatgaccaaaatggatatccgaggtgctgtggatgctgctgtccccaccaatattattgctgccaaggctgcagaagttcgtgcaaacaaagtcaactggcaatcctatcttcagggacagatgatttctgctgaagattgtgagtttattcagaggtttgaaatgaaacgaagccctgaagagaagcaagagatgcttcaaactgaaggcagccagtgtgctaaaacatttataaatctgatgactcatatctgcaaagaacagaccgttcagtatatactaactatggtggatgatatgctgcaggaaaatcatcagcgtgttagcattttctttgactatgcaagatgtagcaagaacactgcgtggccctactttctgccaatgttgaatcgccaggatcccttcactgttcatatggcagcaagaattattgccaagttagcagcttggggaaaagaactgatggaaggcagtgacttaaattactatttcaattggataaaaactcagctgagttcacagaaactgcgtggtagcggtgttgctgttgaaacaggaacagtctcttcaagtgatagttcgcagtatgtgcagtgcgtggccgggtgtttgcagctgatgctccgggtcaatgagtaccgctttgcttgggtggaagcagatggggtaaattgcataatgggagtgttgagtaacaagtgtggctttcagctccagtatcaaatgattttttcaatatggctcctggcattcagtcctcaaatgtgtgaacacctgcggcgctataatatcattccagttctgtctgatatccttcaggagtctgtcaaagagaaagtaacaagaatcattcttgcagcatttcgtaactttttagaaaaatcaactgaaagagaaactcgccaagaatatgccctggctatgattcagtgcaaagttctgaaacagttggagaacttggaacagcagaagtacgatgatgaagatatcagcgaagatatcaaatttcttttggaaaaacttggagagagtgtccaggaccttagttcatttgatgaatacagttcagaacttaaatctggaaggttggaatggagtcctgtgcacaaatctgagaaattttggagagagaatgctgtgaggttaaatgagaagaattatgaactcttgaaaatcttgacaaaacttttggaagtgtcagatgatccccaagtcttagctgttgctgctcacgatgttggagaatatgtgcggcattatccacgaggcaaacgggtcatcgagcagctcggtgggaagcagctggtcatgaaccacatgcatcatgaagaccagcaggtccgctataatgctctgctggccgtgcagaagctcatggtgcacaactgggaataccttggcaagcagctccagtccgagcagccccagaccgctgccgcccgaagctaagcctgcctctggccttcccctccgcctcaatgcagaaccagtagtgggagcactgtgtttagagttaagagtgaacactgtttgattttacttggaatttcctctgttatatagcttttcccaatgctaatttccaaacaacaacaacaaaataacatgtttgcctgttaagttgtataaaagtaggtgattctgtatttaaagaaaatattactgttacatatactgcttgcaatttctgtatttattgttctctggaaataaatatagttattaaaggattctcactccaaacatggcctctctctttacttggactttgaacaaaagtcaactgttgtctcttttcaaaccaaattgggagaattgttgcaaagtagtgaatggcaaataaatgttttaaaatctatcgctctatcaa

SEQ ID NO: 6 - DNA, 3505 bp, Homo sapiens
  source 1..3505: mol_type = other DNA
cgtcaggggcaggggagggacggcgcaggcgcagaaaagggggcggcggactcggcttgttgtgttgctgcctgagtgccggagacggtcctgctgctgccgcagtcctgccagctgtccgacaatgtcgtcccacctagtcgagccgccgccgcccctgcacaacaacaacaacaactgcgaggaaaatgagcagtctctgcccccgccggccggcctcaacagttcctgggtggagctacccatgaacagcagcaatggcaatgataatggcaatgggaaaaatggggggctggaacacgtaccatcctcatcctccatccacaatggagacatggagaagattcttttggatgcacaacatgaatcaggacagagtagttccagaggcagttctcactgtgacagcccttcgccacaagaagatgggcagatcatgtttgatgtggaaatgcacaccagcagggaccatagctctcagtcagaagaagaagttgtagaaggagagaaggaagtcgaggctttgaagaaaagtgcggactgggtatcagactggtccagtagacccgaaaacattccacccaaggagttccacttcagacaccctaaacgttctgtgtctttaagcatgaggaaaagtggagccatgaagaaagggggtattttctccgcagaatttctgaaggtgttcattccatctctcttcctttctcatgttttggctttggggctaggcatctatattggaaagcgactgagcacaccctctgccagcacctactgagggaaaggaaaagcccctggaaatgcgtgtgacctgtgaagtggtgtattgtcacagtagcttatttgaacttgagaccattgtaagcatgacccaacctaccaccctgtttttacatatccaattccagtaactctcaaattcaatattttattcaaactctgttgaggcattttactaaccttataccctttttggcctgaagacattttagaatttcctaacagagtttactgttgtttagaaatttgcaagggcttcttttccgcaaatgccaccagcagattataattttgtcagcaatgctattatctctaattagtgccaccagactagacctgtatcattcatggtataaattttactcttgcaacataactaccatctctctcttaaaacgagatcaggttagcaaatgatgtaaaagaagctttattgtctagttgttttttttcccccaagacaaaggcaagtttccctaagtttgagttgatagttattaaaaagaaaacaaaacaaaaaaaaaaggcaaggcacaacaaaaaaatatcctgggcaataaaaaaaatattttaaaccagctttggagccacttttttgtctaagcctcctaatagcgtcttttaatttataggaggcaaactgtataaatgataggtatgaaatagaataagaagtaaaatacatcagcagattttcatactagtatgttgtaatgctgtcttttctatggtgtagaatctttctttctgataaggaacgtctcaggcttagaaatatatgaaattgctttttgagatttttgcgtgtgtgtttgatattttttacgataattagctgcatgtgaatttttcatgaccttctttacattttttattttttatttctttatttttttttctctaagaagaggctttggaatgagttccaatttgtgatgttaatacaggcttcttgttttaggaagcatcacctatactctgaagcctttaaactctgaagagaattgtttcagagttattccaagcacttgtgcaacttggaaaaacagacttgggttgtgggaacagttgacagcgttctgaaaagatgccatttgtttccttctgatctctcactgaataatgtttactgtacagtcttcccaaggtgattcctgcgactgcaggcactggtcattttctcatgtagctgtcttttcagttatggtaaactcttaaagttcagaacactcaacagattccttcagtgatatacttgttcgttcatttctaaaatgtgaagctttaggaccaaattgttagaaagcatcaggatgaccagttatctcgagtagattttcttggatttcagaacatctagcatgactctgaaggataccacatgttttatatataaataattactgtttatgatatagacattgatattgactatttagagaaccgttgttaattttaaaactagcaatctataaagtgcaccaggtcaacttgaataaaaacactatgacagacaggtttgccagtttgcagaaactaactcttttctcacatcaacatttgtaaaattgatgtgttatagtggaaaataacatatagattaaacaaaatttttatcttttttcaagaatatagctggctatctttaagaaagatgatatatcctagttttgaaagtaattttcttttttctttctagcatttgatgtctaaataattttggacatctttttcctagaccatgtttctgtcttactcttaaacctggtaacacttgatttgccttctataacctatttatttcaagtgttcatatttgaatttctttgggaagaaagtaaatctgatggctcactgatttttgaaaagcctgaataaaattggaaagactggaaagttaggagaactgactagctaaactgctacagtatgcaatttctattacaattggtattacaggggggaaaagtaaaattacactttacctgaaagtgacttcttacagctagtgcattgtgctctttccaagttcagcagcagttctatcagtggtgccactgaaactgggtatatttatgatttctttcagcgttaaaaagaaacatagtgttgccctttttcttaaagcatcagtgaaattatggaaaattacttaaaacgtgaatacatcatcacagtagaatttattatgagagcatgtagtatgtatctgtagccctaacacatgggatgaacgttttactgctacacccagatttgtgttgaacgaaaacattgtggtttggaaaggagaattcaacaattaatagttgaaattgtgaggttaatgtttaaaaagctttacacctgtttacaatttggggacaaaaaggcaggcttcatttttcatatgtttgatgaaaactggctcaagatgtttgtaaatagaatcaagagcaaaactgcacaaacttgcacattggaaagtgcaacaagttcccgtgattgcagtaaaaatatttactattctaaaaaaatgagaattgaagacttagccagtcagataagttttttcatgaacccgttgtggaaattattggaattaactgagccaaagtgattatgcattcttcatctattttagttagcactttgtatcgttatatacagtttacaatacatgtataacttgtagctataaacattttgtgccattaaagctctcacaaaactttaaaaa

SEQ ID NO: 7 - DNA, 2949 bp, Homo sapiens
  source 1..2949: mol_type = other DNA
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaaaa

SEQ ID NO: 8 - DNA, 2260 bp, Homo sapiens
  source 1..2260: mol_type = other DNA
cgcagccccggttcctgcccgcacctctccctccacacctccccgcaagctgagggagccggctccggcctcggccagcccaggaaggcgctcccacagcgcagtggtgggctgaagggctcctcaagtgccgccaaagtgggagcccaggcagaggaggcgccgagagcgagggagggctgtgaggactgccagcacgctgtcacctctcaatagcagcccaaacagattaagacatgggagatgtacaagggcagccgtggggctggcaacagcttcgtaatcctggcttcctgctttctgggtcaaagccctggtggtgtgttcttgatatcggtccatctagtggcgttgtttgattcctcccaccttgctgatcattcgtagtgtagcccccaaggtgtggaataacccttaagcccttaccggggtccttctggactgagaattgttgtaaagtaatactgctcaggtgaaagacaacttgagtggttaaattactgtcatgcaaagcgactagatggttcagctgattgcacctttagaagttatgtggaacgaggcagcagatcttaagccccttgctctgtcacgcaggctggaatgcagtggtggaatcatggctcactacagccctgacctcctgggcccagagatggagtctcgctattttgcccaggttggtcttgaacacctggcttcaagcagtcctcctgcttttggcttcttgaagtgcttggattacagtatttcagttttatgctctgcaacaagtttggccatgttggaggacaatccaaaggtcagcaagttggctactggcgattggatgctcactctgaagccaaagtctattactgtgcccgtggaaatccccagctcccctctggatgatacaccccctgaagactccattcctttggtctttccagaattagaccagcagctacagcccctgccgccttgtcatgactccgaggaatccatggaggtgttcaaacagcactgccaaatagcagaagaataccatgaggtcaaaaaggaaatcaccctgcttgagcaaaggaagaaggagctcattgccaagttagatcaggcagaaaaggagaaggtggatgctgctgagctggttcgggaattcgaggctctgacggaggagaatcggacgttgaggttggcccagtctcaatgtgtggaacaactggagaaacttcgaatacagtatcagaagaggcagggctcgtcctaactttaaatttttcagtgtgagcatacgaggctgatgactgccctgtgctggccaaaagatttttattttaaatgaatagtgagtcagatctattgcttctctgtattacccacatgacaactgtctataatgagtttactgcttgccagcttctagcttgagagaagggatattttaaatgagatcattaacgtgaaactattactagtatatgtttttggagatcagaattcttttccaaagatatatgtttttttcttttttaggaagatatgatcatgctgtacaacagggtagaaaatgataaaaatagactattgactgacccagctaagaatcgtgggctgagcagagttaaaccatgggacaaacccataacatgttcaccatagtttcacgtatgtgtatttttaaatttcatgcctttaatatttcaaatatgctcaaatttaaactgtcagaaacttctgtgcatgtatttatatttgccagagtataaacttttatactctgatttttatccttcaatgattgattatactaagaataaatggtcacatatcctaaaagcttcttcatgaaattattagcagaaaccatgtttgtaaccaaagcacatttgccaatgctaactggctgttgtaataataaacagataaggctgcatttgcttcatgccatgtgacctcacagtaaacatctctgcctttgcctgtgtgtgttctgggggaggggggacatggaaaaatattgtttggacattacttgggtgagtgcccatgaaaacatcagtgaacttgtaactattgttttgttttggatttaaggagatgttttagatcagtaacagctaataggaatatgcgagtaaattcagaattgaaacaatttctccttgttctacctatcaccacattttctcaaattgaactctttgttatatgtccatttctattcatgtaacttctttttcattaaacatggatcaaaactgacaaaaaaaaaaaaaaa

SEQ ID NO: 9 - DNA, 7680 bp, Homo sapiens
  source 1..7680: mol_type = other DNA
ggggccggggggcggagccttgcgggctggagcgaaagaatgcgggggctgagcgcagaagcggctcgaggctggaagaggatcttgggcgccgccagtctttagcaccagttggtgtaggagttgagacctacttcacagtagttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 10 - DNA, 2880 bp, Homo sapiens
  source 1..2880: mol_type = other DNA
gcttccctgggtgccacggtcatgtgacttcggcaagatggctgccctgacagcggagcattttgcagcactccagagcctgctcaagctgctccaggctctgcaccgcctcactaggctggtggcattccgtgacctgtcctctgccgaggcaattctggctctctttccagaaaatttccaccaaaacctcaaaaacctgctgacaaagatcatcctagaacatgtgtctacttggagaaccgaagcccaggcaaatcagatctctctgccacgcctggtcgatctggactggagagtggatatcaaaacctcctcagacagcatcagccgcatggccgtccccacctgcctgctccagatgaagatccaagaagatcccagcctatgcggagacaaaccctccatctcagctgtcaccgtggagctgagcaaagaaacactggacaccatgttagatggcctgggccgcatccgagaccaactctctgccgtggccagtaaatgatccagccagctgccagggccactgccatgacccagctgctcatgagtgataaatgtctccccatatgcaggctgcccttgcagctgcagctgacaacaggcaggatggtggggacagcagggggctactgccatccagaagttacagttggattgggaagaagcagccagatcccccgctgttctcactcatcttctttctctttctgaagctggagagcagaagcccccatctttgaaaagctcctgagtgcaacttaattaccaccatggcagggtgagggaacatttgcatcgtcagctgcctctgcatagctgtttgagaaattcaggcccaaatcatgcagcctatccaataagtaagtttatttccaacattagctctaattagttcatttccaatcccagaacacatggagggaatcggacaggtgatgccagcagttcctgctcctctgtcagggaagccaggcagagcccacagagcatggtccatccagagtgttccctgagccccctccaccatactggaacccctcttcagtgtaggaagtctgaaatgggtgctaattcccttcttcatgaaaccagggccctcttccttcatctaatgcagccactcctaggtgaagaagtgggaataattggaaataaacaacagttctaaaacttccatgatttttgtagcttcttttgtccccaagttgaagcttttggccagtaccttctctagtttttaaagatgatcccaacttcctaattcccagctaagcccttgacccatggtgtgacatgaaatcaggcaattgaatcgcaccactttctgtgttttcacctgttacgtagaacaaaaggaagcaaggtggccaggcgcaatggctcacgcctgtaatcccagcactttgggaggccgaggcaggcagatcatgaggtcaggagatcgagaccatggtgaaaccccatctctactaaaaatacaaaaaattagctgggcgcggtggcgggcatctgtagtcccagctcctcgggaggctgaggcaggagaatggcgtgaacctgggaggcagagcttgcagtgagccgagatcgtgccactgcactccagtctgggtgacagagaaggactcgtctcaaaaaataaaaataaataaaaaggaagcaaggctaatcatcagtatgtgcttgttacaagagctatgatgaaggcactccttcgagtttaaccaaatgagatcatctctgtcatgtgcctcacgcctcacagggactccatgtgtgaagattcccccttcactcaccagatcatctccatggcaacagcttgcagcctgctcttggagtgctttgttttggcagcttctctgctagtttgtgtatggagtgaatggaggaggtaaatccacagattaagaatatgctgtcaggagtcaggcagccaaggtcagaagccagctctgcttctcagtggtaaggtgcttgacttctacatctcaattttcacccactttgtacttttttcctaaattaaatgagtataatagtagtacctacttgataggacttttgtgaaaattaaatgatataatgcacctaaaaacagtactgttacaactaataggaaaggctttgattattaatggatgagagtagaaagcttggtgcatttattgtctcatctactataacagagttggtgtgagaattagtattatcatcctccctttattgaccaggaaaccagctcattgagattgagtcatctgctggtaaatggtctcattaagaggtggacccatatttctctagctttctctttacaacacaggactttgcaaggaacatataattctgtgactagcgccatttggaaaatgttgaaactgaagtagagatgagagatcttacgtctgcctacccagtgagatacgaggaaggtcaagggaaaaaaaattccaagctcttctttatctgctataggaaatgaacattcaattttttgcatgcaacgacaagaggtcaaggaccccagaagccagcccgctacttccaagttgagagcccctggtcataccctccagttgagctcagatttgtcacaaatttacccctctcctttccttccattccccatgacctgcagagagagatgtcagataccttcctcttggcctcccatgggcatccataagaaacttacttgaagcaagaagcccagtataggtgtctgggcagttggacatttcctctagccagatctgtccgaatagagccatctgggtacatgacgcagagggcatttgataaataactggaaaagtcaataaatctttgctacccttcaaaaaaaaaaaaaaaaaaaaaaaaaaaaaaaaa

SEQ ID NO: 11 - DNA, 2358 bp, Homo sapiens
  source 1..2358: mol_type = other DNA
aaactcacacaacaactcttccccgctgagaggagacagccagtgcgactccaccctccagctcgacggcagccgccccggccgacagccccgagacgacagcccggcgcgtcccggtccccacctccgaccaccgccagcgctccaggccccgccgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcaaccccagacactggtttgaagaatgttaagacttgacagtggaactacattagtacacagcaccagaatgtatattaaggtgtggctttaggagcagtgggagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtccttggcaggctgatttctaggtaggaaatgtggtagcctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtgtatattttttctataaaaaaaaaaaaaaaaa

SEQ ID NO: 12 - DNA, 4697 bp, Homo sapiens
  source 1..4697: mol_type = other DNA
agacgctcgcctggcagctgcgcacactcggagcgccccgagcggcgcagatagggacgttggggctgtgccccgcggcgcggcgcctgccactgcgcaggcgcctcaggaagagctcggcatcgcccctcttcctccaggtcccccttccccgcaacttcccacgagtgccaggtgccgcgagcgccgagttccgcgcattggaaagaagcgaccgcggcggctggaaccctgattgctgtccttcaacgtgttcattatgaagttattagtaatacttttgttttctggacttataactggttttagaagtgactcttcctctagtttgccacctaagttactactagtatcctttgatggcttcagagctgattatctgaagaactatgaatttcctcatctccagaattttatcaaagaaggtgttttggtagagcatgttaaaaatgtttttatcacaaaaacatttccaaaccactacagtattgtgacaggcttgtatgaagaaagccatggcattgtggctaattccatgtatgatgcagtcacaaagaaacacttttctgactctaatgacaaggatcctttttggtggaatgaggcagtacctatttgggtgaccaatcagcttcaggaaaacagatcaagtgctgctgctatgtggcctggtactgatgtacccattcacgataccatctcttcctattttatgaattacaactcctcagtgtcatttgaggaaagactaaataatattactatgtggctaaacaattcgaacccaccagtcacctttgcaacactatattgggaagaaccagatgcaagtggccacaaatacggacctgaagataaagaaaacatgagcagagtgttgaaaaaaatagatgatcttatcggtgacttagtccaaagactcaagatgttagggctatgggaaaatcttaatgtgatcattacaagtgatcatgggatgacccagtgttctcaggacagactgataaacctggattcctgcatcgatcattcatactacactcttatagatttgagcccagttgctgcaatacttcccaaaataaatagaacagaggtttataacaaactgaaaaactgtagccctcatatgaatgtttatctcaaagaagacattcctaacagattttattaccaacataatgatcgaattcagcccattattttggttgccgatgaaggctggacaattgtgctaaatgaatcatcacaaaaattaggtgaccatggttatgataattctttgcctagtatgcatccatttctagctgcccacggacctgcatttcacaaaggctacaagcatagcacaattaacattgtggatatttatccaatgatgtgccacatcctgggattaaaaccacatcccaataatgggacctttggtcatactaagtgcttgttagttgaccagtggtgcattaatctcccagaagccatcgcgattgttatcggttcactcttggtgttaaccatgctaacatgcctcataataatcatgcagaatagactttctgtacctcgtccattttctcgacttcagctacaagaagatgatgatgatcctttaattgggtgacatgtgctagggcttatacaaagtgtctttgattaatcacaaaactaagaatacatccaaagaatagtgttgtaactatgaaaaagaatactttgaaagacaaagaacttagactaagcatgttaaaattattactttgttttccttgtgttttgtttcggtgcatttgctaataagataacgctgaccatagtaaaattgttagtaaatcattaggtaacatcttgtggtaggaaatcattaggtaacatcaatcctaactagaaatactaaaaatggcttttgagaaaaatacttcctctgcttgtattttgcgatgaagatgtgatacatctttaaatgaaaatataccaaaatttagtaggcatgtttttctaataaatttatatatttgtaaagaaaacaacagaaatctttatgcaatttgtgaattttgtatattagggaggaaaagcttcctatatttttatatttacctttaattagtttgtatctcaagtaccctcttgaggtaggaaatgctctgtgatggtaaataaaattggagcagacagaaaagatatagcaaatgaagaaatattttaaggaaacctatttgaaaaaaaaagcaaagaccatttgataaaagcctgagttgtcaccattatgtcttaagctgttagtcttaaagattattgttaaaaaattcagaagaaaagagagacaagtgctcttctctctatctatgcttaatgcctttatgtaagttacttagttgtttgcgtgtgcctgtgcaagtgtgtttgtgtgtggttgtgtggacattatgtgatttactatataaggaggtcagagatggactgtggccaggcttccacattcctgaagcacacagatctcaggaaaggttatttttgcacttcatatttgtttactttctcctaactcacaagttaaaatcataacttaatttcattaacttttatcatttaactctctcatgtttgttgtaacctgaggtatccaaatgctacagaaaaatttatgacccaaatacaaatctcaatttgactgggacagaatgaggaatggagatttttgtatttatctttgggactttatgccttactttttaggctatagaatagttaagaaattttaaacaaaatttagtatcttttggtctttcacaccattcatatgttaagtggcagaatagccttagtgctacctccacttttttctccagtatttgcatcacagaaataatccctctgtttaacatgtttgttcagagccaagggtttattgtgaagaactgtcatcctgcctttgctagctggtaccttctagtaatcaaaattaatatgaagaaactaggttgtgacagactagattatatttagtaggggaaaaattgggctcaagaaccattcatcagtacgtgagacaagcagttaatagtatgatctttaaagttttgacaatataaaataaacttggtaactgttttacaaatataaaagtataataaatatgcagcccagttaaatattgattatctgtgatggtaaagaacaacagtggtgccagtcatcaaacatacagtgcgtcctattgagtcactgctaatttcttgagcctggtatttgctgcctattgtatttgtggttgttgagaggcattttcaaaccctgtataaataatccatgctgttggtcataagttaactgtattaagaacagtaaaataaataaaaaccaatagtactaattttgctttaaaaaaatttctaatttttttcacataaaacaattatcctaaaggttaatagttgatcgaaacagaataatagaaaaattctactttaatttccattaaaaagcaaatagcattgacacatttaaagcttttcatttaaagtagtggatgtttttgaagtatctaaaatagtagcagaatattttatacttggtccttgcaatggtgtgagttttaatgattgcattatcgtgattggtggttatgagtttcagaaatctatacttggcatccaactcatgagtggattttatataggatggaacaggaaggtatgtcctgtcagtatcttaaccctttcaacaagacatttacctatttgtctttccttacgttctcaaaatattaactcgaattgtaaattaagcaaaaatttaaaaagtatatgttgatgggacaagaagaatagtatttatttaataaaacatatattatattgaactatgtgttaattcatttgtatcttttaaaaaattatcactgttaaagccattgactcctttagtacactgagaaaaatcttatagtaaaactagcctttcacattaaggttttggtgtgtattttgttaaataactaacatgctgctctattttctgggtgtagaaagtatttggctctaggaaacatttacttgtttgtgaaaacaataccccaaggtaataggaaaagtttgagttaagtgtttttaattcagtcagtgaattcagaataagtacattcatgtataacatagggacagttctgctgctgttatttatatgcaattcttctggtaaatagcaatagaataaaacatatttcaatgtttgtgtataggttttatattattattccactaggaatggcataagaatttatagataaattcttgtaacattaaaggattaaaatgtttttacattgtttttgggtgtctccttcttgtgcccatatctgataagctttatggattattgcatttaattccttttatttggagggttttacttccttgttaacatataaagttataaatgaaggacaaggaggagatggaaaatgtgtatttattgttaattcttaaaatagtgtgtaaataaaataacatcagtgtgctttaaagaaatgtgtatgtagtgccttaatttaaattaaaatatttttgactgttacttgagttcagaattaatgactttgttcatgatttttaaaatgtgtgtgaataaaatctaccaaaaaattcttactgtaattattaaatataaagttcagtgtcaaaaaaaaaaaaaaaaaa

SEQ ID NO: 13 - DNA, 2448 bp, Homo sapiens
  source 1..2448: mol_type = other DNA
accggcccggttccctctccggggagcggcggcggacgcgcggctcccacccctcccctctcacgggctctcccctccccagtgtggccgcgaccctaccctctgcaaggcgatggcccgcgccccgagcgcaggctagcgtgcctgggtgcccggccatgggctgtatcggctctcggagcccggcgggtcaggcatttctggggaccaacagctggccgaggctcagggatagagacggctgctccagctaaaggtgaatgttggagacacagtcgcgatgctgcccaagtcccggcgagccctaactatccaggagatcgctgcgctggccaggtcctccctgcatggtatttcccaggtggtgaaggaccacgtgaccaagcctaccgccatggcccagggccgagtggctcacctcattgagtggaagggctggagcaagccgagtgactcacctgctgccctggaatcagccttttcctcctattcagacctcagcgagggcgaacaagaggctcgctttgcagcaggagtggctgagcagtttgccatcgcggaagccaagctccgagcatggtcttcggtggatggcgaggactccactgatgactcctatgatgaggactttgctgggggaatggacacagacatggctgggcagctgcccctggggccgcacctccaggacctgttcaccggccaccggttctcccggcctgtgcgccagggctccgtggagcctgagagcgactgctcacagaccgtgtccccagacaccctgtgctctagtctgtgcagcctggaggatgggttgttgggctccccggcccggctggcctcccagctgctgggcgatgagctgcttctcgccaaactgccccccagccgggaaagtgccttccgcagcctgggcccactggaggcccaggactcactctacaactcgcccctcacagagtcctgcctttcccccgcggaggaggagccagccccctgcaaggactgccagccactctgcccaccactaacgggcagctgggaacggcagcggcaagcctctgacctggcctcttctggggtggtgtccttagatgaggatgaggcagagccagaggaacagtgacccacatcatgcctggcagtggcatgcatcccccggctgctgccaggggcagagcctctgtgcccaagtgtgggctcaaggctcccagcagagctccacagcctagagggctcctgggagcgctcgcttctccgttgtgtgttttgcatgaaagtgtttggagaggaggcaggggctgggctgggggcgcatgtcctgcccccactcccggggcttgccgggggttgcccggggcctctggggcatggctacagctgtggcagacagtgatgttcatgttcttaaaatgccacacacacatttcctcctcggataatgtgaaccactaagggggttgtgactgggctgtgtgagggtggggtgggagggggcccagcaaccccccaccctccccatgcctctctcttctctgcttttcttctcacttccgagtccatgtgcagtgcttgatagaatcacccccacctggaggggctggctcctgccctcccggagcctatgggttgagccgtccctcaagggcccctgcccagctgggctcgtgctgtgcttcattcacctctccatcgtctctaaatcttcctcttttttcctaaagacagaaggtttttggtctgttttttcagtcggatcttctcttctctgggaggctttggaatgatgaaagcatgtaccctccacccttttcctggccccctaatggggcctgggccctttcccaacccctcctaggatgtgcgggcagtgtgctggcgcctcacagccagccgggctgcccattcacgcagagctctctgagcgggaggtggaagaaaggatggctctggttgccacagagctgggacttcatgttcttctagagagggccacaagagggccacaggggtggccgggagttgtcagctgatgcctgctgagaggcaggaattgtgccagtgagtgacagtcatgagggagtgtctcttcttggggaggaaagaaggtagagcctttctgtctgaatgaaaggccaaggctacagtacagggccccaccccagccagggtgttaatgcccacgtagtggaggcctctggcagatcctgcattccaaggtcactggactgtacgtttttatggttgtgggaagggtgggtggctttagaattaagggccttgtaggctttggcaggtaagagggcccaaggtaagaacgagagccaacgggcacaagcattctatatataagtggctcattaggtgtttattttgttctatttaagaatttgttttattaaattaatataaaaatctttgtaaatctctaaaaaaaaaaaaaaaaaa

SEQ ID NO: 14 - DNA, 5948 bp, Homo sapiens
  source 1..5948: mol_type = other DNA
ggagcgggccgagccgccaccgcggccggagctgtcccttagccagacccggcgagacacgagcggcgggagggaggcggtggcgcgcccggccccgcccgcccgaccaagcgtcggacgcggcccggcgccgagccatggagcctgagccagtggaggactgtgtgcagagcactctcgccgccctgtatccaccctttgaggcaacagcccccaccctgttgggccaggtgttccaggtggtggagaggacttatcgggaggacgcactgaggtacacgctggacttcctggtaccagccaagcacctgcttgccaaggtccagcaggaagcctgtgcccaatacagtggattcctcttcttccatgaggggtggccgctctgcctgcatgaacaggtggtggtgcagctagcagccctaccctggcaactgctgcgcccaggagacttctatctgcaggtggtgccctcagctgcccaagcaccccgactagcactcaagtgtctggcccctgggggtgggcgggtgcaggaggttcctgtgcccaatgaggcttgtgcctacctattcacacctgagtggctacaaggcatcaacaaggaccggccaacaggtcgcctcagtacctgcctactgtctgcgccctctgggattcagcggctgccctgggctgagctcatctgtccacgatttgtgcacaaagagggcctcatggttggacatcagccaagtacactgcccccagaactgccctctggacctccagggcttcccagccctccacttcctgaggaggcgctgggtacccggagtcctggggatgggcacaatgcccctgtggaaggacctgagggcgagtatgtggagctgttagaggtgacgctgcccgtgagggggagcccaacagatgctgaaggctccccaggcctctccagagtccggacggtacccacccgcaagggcgctggagggaagggccgccaccggagacaccgggcgtggatgcaccagaagggcctggggcctcggggccaggatggagcacgcccacccggcgaggggagcagcaccggagcctcccctgagtctcccccaggagctgaggctgtcccagaggcagcagtcttggaggtgtctgagcccccagcagaggctgtgggagaagcctccggatcttgccccctgaggccaggggagcttagaggaggaggaggaggaggccagggggctgaaggaccacctggtacccctcggagaacaggcaaaggaaacagaagaaagaagcgagctgcaggtcgaggggctcttagccgaggaggggacagtgccccactgagccctggggacaaggaagatgccagccaccaagaagcccttggcaatctgccctcaccaagtgagcacaagcttccagaatgccacctggttaaggaggaatatgaaggctcagggaagccagaatctgagccaaaagagctcaaaacagcaggcgagaaagagcctcagctctctgaagcctgtgggcctacagaagagggggccggagagagagagctggaggggccaggcctgctgtgtatggcaggacacacaggcccagaaggccccctgtctgacactccaacacctccgctggagactgtgcaggaaggaaaaggggacaacattccagaagaggcccttgcagtctccgtctctgatcaccctgatgtagcttgggacttgatggcatctggattcctcatcctgacgggaggggtggaccagagtgggcgagctctgctgaccattaccccaccgtgccctcctgaggagcccccaccctcccgagacacgctgaacacaactcttcattacctccactcactgctcaggcctgatctacagacactggggctgtccgtcctgctggaccttcgtcaggcacctccactgcctccagcactcattcctgccttgagccaacttcaggactcaggagatcctccccttgttcagcggctgctgattctcattcatgatgaccttccaactgaactctgtggatttcagggtgctgaggtgctgtcagagaatgatctgaaaagagtggccaagccagaggagctgcagtgggagttaggaggtcacagggacccctctcccagtcactgggtagagatacaccaggaagtggtaaggctatgtcgcctgtgccaaggtgtgctgggctcggtacggcaggccattgaggagctggagggagcagcagagccagaggaagaggaggcagtgggaatgcccaagccactgcagaaggtgctggcagatccccggctgacggcactgcagagggatgggggggccatcctgatgaggctgcgctccactcccagcagcaagctggagggccaaggcccagctacactgtatcaggaagtggacgaggccattcaccagcttgtgcgcctctccaacctgcacgtgcagcagcaagagcagcggcagtgcctgcggcgactccagcaggtgttgcagtggctctcgggcccaggggaggagcagctggcaagctttgctatgcctggggacaccttgtctgccctgcaggagacagagctgcgattccgtgctttcagcgctgaggtccaggagcgcctggcccaggcacgggaggccctggctctggaggagaatgccacctcccagaaggtgctggatatctttgaacagcggctggagcaggttgagagtggcctccatcgggccctgcggctacagcgcttcttccagcaggcacatgaatgggtggatgagggctttgctcggctggcaggagctgggccgggtcgggaggctgtgctggctgcactggccctgcggcgggccccagagcccagtgccggcaccttccaggagatgcgggccctggccctggacctgggcagcccagcagccctgcgagaatggggccgctgccaggcccgctgccaagagctagagaggaggatccagcaacacgtgggagaggaggcgagcccacggggctaccgacgacggcgggcagacggtgccagcagtggaggggcccagtgggggccccgcagcccctcgcccagcctcagctccttgctgctccccagcagccctgggccacggccagccccatcccattgctccctggccccatgtggagaggactatgaggaagagggccctgagctggctccagaagcagagggcaggcccccaagagctgtgctgatccgaggcctggaggtcaccagcactgaggtggtagacaggacgtgctcaccacgggaacacgtgctgctgggccgggctagggggccagacggaccctggggagtaggcaccccccggatggagcgcaagcgaagcatcagtgcccagcagcggctggtgtctgagctgattgcctgtgaacaagattacgtggccaccttgagtgagccagtgccaccccctgggcctgagctgacgcctgaacttcggggcacctgggctgctgccctgagtgcccgggaaaggcttcgcagcttccaccggacacactttctgcgggagcttcagggctgcgccacccaccccctacgcattggggcctgcttccttcgccacggggaccagttcagcctttatgcacagtacgtgaagcaccgacacaaactggagaatggtctggctgcgctcagtcccttaagcaagggctccatggaggctggcccttacctgccccgagccctgcagcagcctctggaacagctgactcggtatgggcggctcctggaggagctcctgagggaagctgggcctgagctcagttctgagtgccgggcccttggggctgctgtacagctgctccgggaacaagaggcccgtggcagagacctgctggccgtggaggcggtgcgtggctgtgagatagatctgaaggagcagggacagctcttgcatcgagaccccttcactgtcatctgtggccgaaagaagtgccttcgccatgtctttctcttcgagcatctcctcctgttcagcaagctcaagggccctgaaggggggtcagagatgtttgtttacaagcaggcctttaagactgctgatatggggctgacagaaaacatcggggacagcggactctgctttgagttgtggtttcggcggcggcgtgcacgagaggcatacactctgcaggcaacctcaccagagatcaaactcaagtggacaagttctattgcccagctgctgtggagacaggcagcccacaacaaggagctccgagtgcagcagatggtgtccatgggcattgggaataaacccttcctggacatcaaagcccttggggagcggacgctgagtgccctgctcactggaagagccgcccgcacccgggcctccgtggccgtgtcatcctttgagcatgccggcccctcccttcccggcctttcgccgggagcctgctccctgcctgcccgcgtcgaggaggaggcctgggatctggacgtcaagcaaatttccctggccccagaaacacttgactcttctggagatgtgtccccaggaccaagaaacagccccagcctgcaacccccccaccctgggagcagcactcccaccctggccagtcgagggatcttagggctatcccgacagagtcatgctcgagccctgagtgaccccaccacgcctctgtgacctggagaagatccagaacttgcgtgcagcttctcctctcagcacactttgggctgggatggcagtggggcataatggagccctgggcgatcgctgaatttcttccctctgcttcctggacacagaggaggtctaacgaccagagtattgccctgccaccactatctctagtctccctagcttggtgccttctcctgcaggagtcagagcagccacattgcttgccttcataccctggaggtggggaagttatccctcttccggtgctttcccatcctgggccactgtatccaggacatcactcccatgccagccctccctggcagcccatgttctcctcttttctcaccccctgactttccctgagaagaatcatctctgccaggtcaactggagtccctggtgactccattctgaggtgtcacaagcaatgaagctatgcaaacaataggagggtgtgacaggggaaccgtagactttatatatgtaattactgttattataatactattgttatattaaatgtatttactcacactttgcctctaaggagctagagtagtcctctggattaaggtgataaataacttgagcactttccctcaaccagcccttaactagaacacagaaaataaaaccaagactggaaggtcccctctacccctcccaggcccagagctagctgactgtgtatgagcctgggagaatgtgtctcctccacagtggctcccagaggttccacacactctctgaagctccttctcccacactgcacctactccttgaggctgaactggtcacagacaaactgggatccagcacagtccagcagttctcaaaatgaggtcctcaggccacagtgcgtgagaacttgcttggctgtttgttaaatgctaattcttgggccccatcagagctactgcatcgaaacctgggggtaaaacccaatattctgcatttcttatcaaactctttgggtgataactaagtgtctgaagaggtgactatttcctgacagaaggacccaaagagggaagcaggacataggtaggcagacagacacagggccctgtgcctcaagacacctgtttattggggacacgactctgcaatagggatgacaggaatcgtaccaaaaatagcgacgtctacagggcccctgatggggctagaagggtacagtgccccccaccctcaccccttgtacaaaaataaactctcacgcctatggaccagcaaaaaaaaaaaaaa

SEQ ID NO: 15 - DNA, 3851 bp, Homo sapiens
  source 1..3851: mol_type = other DNA
ctctcccaaccgcctcgtcgcactcctcaggctgagagcaccgctgcactcgcggccggcgatgcgggaccccggcgcggccgctccgctttcgtccctgggcctctgtgccctggtgctggcgctgctgggcgcactgtccgcgggcgccggggcgcagccgtaccacggagagaagggcatctccgtgccggaccacggcttctgccagcccatctccatcccgctgtgcacggacatcgcctacaaccagaccatcctgcccaacctgctgggccacacgaaccaagaggacgcgggcctcgaggtgcaccagttctacccgctggtgaaggtgcagtgttctcccgaactccgctttttcttatgctccatgtatgcgcccgtgtgcaccgtgctcgatcaggccatcccgccgtgtcgttctctgtgcgagcgcgcccgccagggctgcgaggcgctcatgaacaagttcggcttccagtggcccgagcggctgcgctgcgagaacttcccggtgcacggtgcgggcgagatctgcgtgggccagaacacgtcggacggctccgggggcccaggcggcggccccactgcctaccctaccgcgccctacctgccggacctgcccttcaccgcgctgcccccgggggcctcagatggcagggggcgtcccgccttccccttctcatgcccccgtcagctcaaggtgcccccgtacctgggctaccgcttcctgggtgagcgcgattgtggcgccccgtgcgaaccgggccgtgccaacggcctgatgtactttaaggaggaggagaggcgcttcgcccgcctctgggtgggcgtgtggtccgtgctgtgctgcgcctcgacgctctttaccgttctcacctacctggtggacatgcggcgcttcagctacccagagcggcccatcatcttcctgtcgggctgctacttcatggtggccgtggcgcacgtggccggcttccttctagaggaccgcgccgtgtgcgtggagcgcttctcggacgatggctaccgcacggtggcgcagggcaccaagaaggagggctgcaccatcctcttcatggtgctctacttcttcggcatggccagctccatctggtgggtcattctgtctctcacttggttcctggcggccggcatgaagtggggccacgaggccatcgaggccaactcgcagtacttccacctggccgcgtgggccgtgcccgccgtcaagaccatcactatcctggccatgggccaggtagacggggacctgctgagcggggtgtgctacgttggcctctccagtgtggacgcgctgcggggcttcgtgctggcgcctctgttcgtctacctcttcataggcacgtccttcttgctggccggcttcgtgtccctcttccgtatccgcaccatcatgaaacacgacggcaccaagaccgagaagctggagaagctcatggtgcgcatcggcgtcttcagcgtgctctacacagtgcccgccaccatcgtcctggcctgctacttctacgagcaggccttccgcgagcactgggagcgcacctggctcctgcagacgtgcaagagctatgccgtgccctgcccgcccggccacttcccgcccatgagccccgacttcaccgtcttcatgatcaagtacctgatgaccatgatcgtcggcatcaccactggcttctggatctggtcgggcaagaccctgcagtcgtggcgccgcttctaccacagacttagccacagcagcaagggggagactgcggtatgagccccggcccctccccacctttcccaccccagccctcttgcaagaggagaggcacggtagggaaaagaactgctgggtgggggcctgtttctgtaactttctccccctctactgagaagtgacctggaagtgagaagttctttgcagatttggggcgaggggtgatttggaaaagaagacctgggtggaaagcggtttggatgaaaagatttcaggcaaagacttgcaggaagatgatgataacggcgatgtgaatcgtcaaaggtacgggccagcttgtgcctaatagaaggttgagaccagcagagactgctgtgagtttctcccggctccgaggctgaacggggactgtgagcgatccccctgctgcagggcgagtggcctgtccagacccctgtgaggccccgggaaaggtacagccctgtctgcggtggctgctttgttggaaagagggagggcctcctgcggtgtgcttgtcaagcagtggtcaaaccataatctcttttcactggggccaaactggagcccagatgggttaatttccagggtcagacattacggtctctcctcccctgccccctcccgcctgtttttcctcccgtactgctttcaggtcttgtaaaataagcatttggaagtcttgggaggcctgcctgctagaatcctaatgtgaggatgcaaaagaaatgatgataacattttgagataaggccaaggagacgtggagtaggtatttttgctactttttcattttctggggaaggcaggaggcagaaagacgggtgttttatttggtctaataccctgaaaagaagtgatgacttgttgcttttcaaaacaggaatgcatttttccccttgtctttgttgtaagagacaaaagaggaaacaaaagtgtctccctgtggaaaggcataactgtgacgaaagcaacttttataggcaaagcagcgcaaatctgaggtttcccgttggttgttaatttggttgagataaacattcctttttaaggaaaagtgaagagcagtgtgctgtcacacaccgttaagccagaggttctgacttcgctaaaggaaatgtaagaggttttgttgtctgttttaaataaatttaattcggaacacatgatccaacagactatgttaaaatattcagggaaatctctcccttcatttactttttcttgctataagcctatatttaggtttcttttctatttttttctcccatttggatcctttgaggtaaaaaaacataatgtcttcagcctcataataaaggaaagttaattaaaaaaaaaaagcaaagagccattttgtcctgttttcttggttccatcaatctgtttattaaacatcatccatatgctgaccctgtctctgtgtggttgggttgggaggcgatcagcagataccatagtgaacgaagaggaaggtttgaaccatgggccccatctttaaagaaagtcattaaaagaaggtaaacttcaaagtgattctggagttctttgaaatgtgctggaagacttaaatttattaatcttaaatcatgtactttttttctgtaatagaactcggattcttttgcatgatggggtaaagcttagcagagaatcatgggagctaacctttatcccacctttgacactaccctccaatcttgcaacactatcctgtttctcagaacagtttttaaatgccaatcatagagggtactgtaaagtgtacaagttactttatatatgtaatgttcacttgagtggaactgctttttacattaaagttaaaatcgatcttgtgtttcttcaaccttcaaaactatctcatctgtcagatttttaaaactccaacacaggttttggcatcttttgtgctgtatcttttaagtgcatgtgaaatttgtaaaatagagataagtacagtatgtatattttgtaaatctcccatttttgtaagaaaatatatattgtatttatacatttttactttggatttttgttttgttggctttaaaggtctaccccactttatcacatgtacagatcacaaataaatttttttaaatac

SEQ ID NO: 16 - DNA, 2394 bp, Homo sapiens
  source 1..2394: mol_type = other DNA
gacgggccggtacagcccgtgtccccgccccgcgccatcgctaggcgacgtgcgcttttgccgcgccgtgctgcccgcgagggcagctgaggtggtggtggcggccgccttgtcgaggcatcgcgcgcccgtgaagtgttcgccgtcagtgctgttgggtgcctggagccgcgtcccccgtcccgaaaactgtccttgacagtacttgcgcggcccaacggccgccggcgcccccgcgtctccatggcgacggcctttttccctgcgaggaccccggcggcagggctgccccgcggcgcctgcttggcgcgacgctctagcggttaccgctgcgggctggctgggcgtagtggggctgcgcggctgccacggagctagagggcaagtgtgctcggcccagcgtgcagggaacgcgggcggccagacaacgggctgggctccggggcctgcggcgcgggcgctgagctggcagggcgggtcggggcgcgggctgcatccgcatctcctccatcgcctgcagtaagggcggccgcggcgagcctttgaggggaacgacttgtcggagccctaaccaggggtatctctgagcctggtgggatccccggagcgtcacatcactttccgatcacttcaaagtacagcagaccgaggacacggttgttaccaagaccaggctgttgccttggaagagcccagagcgtgtcaagggagacagccacatcacgccagaaatacatgacagctggattagccctgggagagggaggcccagatgtgggagctcaggggaggtgcagctcaacgtggagtttggaggaggctaccttgacctttgaatgccaagtgggagccagccagatgaaaggggttaaaaactaatatttatatgacagaagaaaaagatgtcattccgtaaagtaaacatcatcatcttggtcctggctgttgctctcttcttactggttttgcaccataacttcctcagcttgagcagtttgttaaggaatgaggttacagattcaggaattgtagggcctcaacctatagactttgtcccaaatgctctccgacatgcagtagatgggagacaagaggagattcctgtggtcatcgctgcatctgaagacaggcttgggggggccattgcagctataaacagcattcagcacaacactcgctccaatgtgattttctacattgttactctcaacaatacagcagaccatctccggtcctggctcaacagtgattccctgaaaagcatcagatacaaaattgtcaattttgaccctaaacttttggaaggaaaagtaaaggaggatcctgaccagggggaatccatgaaacctttaacctttgcaaggttctacttgccaattctggttcccagcgcaaagaaggccatatacatggatgatgatgtaattgtgcaaggtgatattcttgccctttacaatacagcactgaagccaggacatgcagctgcattttcagaagattgtgattcagcctctactaaagttgtcatccgtggagcaggaaaccagtacaattacattggctatcttgactataaaaaggaaagaattcgtaagctttccatgaaagccagcacttgctcatttaatcctggagtttttgttgcaaacctgacggaatggaaacgacagaatataactaaccaactggaaaaatggatgaaactcaatgtagaagagggactgtatagcagaaccctggctggtagcatcacaacacctcctctgcttatcgtattttatcaacagcactctaccatcgatcctatgtggaatgtccgccaccttggttccagtgctggaaaacgatattcacctcagtttgtaaaggctgccaagttactccattggaatggacatttgaagccatggggaaggactgcttcatatactgatgtttgggaaaaatggtatattccagacccaacaggcaaattcaacctaatccgaagatataccgagatctcaaacataaagtgaaacagaatttgaactgtaagcaagcatttctcaggaagtcctggaagatagcatgcgtgggaagtaacagttgctaggcttcaatgcctatcggtagcaagccatggaaaaagatgtgtcagctaggtaaagatgacaaactgccctgtctggcagtcagcttcccagacagactatagactataaatatgtctccatctgccttaccaagtgttttcttactacaatgctgaatgactggaaagaagaactgatatggctagttcagctagctggtacagataattcaaaactgctgttggttttaattttgtaacctgtggcctgatctgtaaataaaacttacatttttcaataggtaaaaaaaaaaaaaaaa

SEQ ID NO: 17 - DNA, 4463 bp, Homo sapiens
  source 1..4463: mol_type = other DNA
gcagcgcgcaccgagccggccgcgccgcgcccgccgctctcgccgctttcgccgcggtctcctcctctagcgcccgccgcggccggtaaatctcggctggaggagcagcggcggcccccgagtcaactttcattccctttttgcttctgcctcaccattctcttctcctcctcgaaagatggctgtttggagaagggggagaagttaagaggtcgccagcgcggagcgaaggagggcgcgatagcctcagcaggagcgggcggaggtttctcctctgccaacccctcctggaccattgtcagcagttgaacgacaaaggctgtgaatctgcatcctagtcttagcagtccctctgattctcatgatgagctcacctgcacagcctgacctcatgtggaaccttgtaccatgggtgctattctgtggctgctgtaggatcttcccagatggggtggctggacgagagcagctcttggctcagcaaagaatgcacagtatgatcagctcagtggatgtgaagtcagaagttcctgtgggcctggagcccatctcacctttagacctaaggacagacctcaggatgatgatgcccgtggtggaccctgttgtccgtgagaagcaattgcagcaggaattacttcttatccagcagcagcaacaaatccagaagcagcttctgatagcagagtttcagaaacagcatgagaacttgacacggcagcaccaggctcagcttcaggagcatatcaaggaacttctagccataaaacagcaacaagaactcctagaaaaggagcagaaactggagcagcagaggcaagaacaggaagtagagaggcatcgcagagaacagcagcttcctcctctcagaggcaaagatagaggacgagaaagggcagtggcaagtacagaagtaaagcagaagcttcaagagttcctactgagtaaatcagcaacgaaagacactccaactaatggaaaaaatcattccgtgagccgccatcccaagctctggtacacggctgcccaccacacatcattggatcaaagctctccaccccttagtggaacatctccatcctacaagtacacattaccaggagcacaagatgcaaaggatgatttcccccttcgaaaaactgaatcctcagtcagtagcagttctccaggctctggtcccagttcaccaaacaatgggccaactggaagtgttactgaaaatgagacttcggttttgccccctacccctcatgccgagcaaatggtttcacagcaacgcattctaattcatgaagattccatgaacctgctaagtctttatacctctccttctttgcccaacattaccttggggcttcccgcagtgccatcccagctcaatgcttcgaattcactcaaagaaaagcagaagtgtgagacgcagacgcttaggcaaggtgttcctctgcctgggcagtatggaggcagcatcccggcatcttccagccaccctcatgttactttagagggaaagccacccaacagcagccaccaggctctcctgcagcatttattattgaaagaacaaatgcgacagcaaaagcttcttgtagctggtggagttcccttacatcctcagtctcccttggcaacaaaagagagaatttcacctggcattagaggtacccacaaattgccccgtcacagacccctgaaccgaacccagtctgcacctttgcctcagagcacgttggctcagctggtcattcaacagcaacaccagcaattcttggagaagcagaagcaataccagcagcagatccacatgaacaaactgctttcgaaatctattgaacaactgaagcaaccaggcagtcaccttgaggaagcagaggaagagcttcagggggaccaggcgatgcaggaagacagagcgccctctagtggcaacagcactaggagcgacagcagtgcttgtgtggatgacacactgggacaagttggggctgtgaaggtcaaggaggaaccagtggacagtgatgaagatgctcagatccaggaaatggaatctggggagcaggctgcttttatgcaacaggtaataggcaaagatttagctccaggatttgtaattaaagtcattatctgaacatgaaatgcattgcaggtttggtaaatggatatgatttcctatcagtttatatttctctatgatttgagttcagtgtttaaggattctacctaatgcagatatatgtatatatctatatagaggtctttctatatactgatctctatatagatatcaatgtttcattgaaaatccactggtaaggaaatacctgttatactaaaattatgatacataatatctgagcagttaataggctttaaatttatcccaaagcctgctacaccaattacttctaaagaaaacaaattcactgttattttgagtttatgtgttgagatcagtgactgctggatagtctcccagtctgatcaatgaagcattcgattagtttttgattttttgcaacatctagaatttaattttcacatcactgtacataatgtatcatactatagtcttgaacactgttaaaggtagtctgccccttccttcctctctctttttttagttaagtagaaatgttctggtcaccatgccagtagtcctaggttattgtgtaggttgcaattgaacatattaggaatacaggtggttttaaatatatagatgcaaattgcagcactactttaaatattagattatgtctcacatagcactgctcattttacttttattttgtgtaatttgatgacactgtctatcaaaaaagagcaaatgaagcagatgcaaatgttagtgagaagtaatgtgcagcattatggtccaatcagatacaatattgtgtctacaattgcaaaaaacacagtaacaggatgaatattatctgatatcaagtcaaaatcagtttgaaaagaaggtgtatcatattttatattgtcactagaatctcttaagtataattccataatgacatgggcatataccgtaacattctggcaaataacaattagaaaagataggtttaacaaaaaaatttacttgtatataatgcaccttcaggaggactatgtcctttgatgctataaaatacaaacaactttgaaggcaacagaagacactgtttattcaagtcagttctttgtcaggttcctgctgttctcctacagaaaagtgattctgtgagggtgaacaggaaatgccttgtggaaacaggaagtccaagtgattcatgtactgaggaatgtaggaaaaaaaatctgaggatagtgctttactctttctgtttttaaagggcactctatgaattgatttattgtctaagaaaataacaccacaagtagggaaattgttacggaagcttttcactggaacatttccttcatattcccttttgatatgtttaccttgttttataggtttacttttgttaagctagttaaaggttcgttgtattaagacccctttaatatggataatccaaattgacctagaatctttgtgaggttttttctattaaaatatttatatttctaaatccgaggtatttcaaggtgtagtatcctatttcaaaggagatatagcagttttgccaaatgtagacattgttcaactgtatgttattggcacgtgttgtttacattttgctgtgacatttaaaaatatttctttaaaaatgttactgctaaagatacattatccttttttaaaaagtctccattcaaattaaattaacataactagaagttagaaagtttaaaagttttccacataatgaaagtccttctgataatttgacaaatagctataataggaacactccctatcaccaacatattttggttagtatattccttcatattaaaatgactttttgtcagttgttttgcattaaaaatatggcatgcctaagataaaattgtatattttttccatctcataaatattcattttcttcaaagtcttttttcaatctcataaaaaagggatagtgcatcttttaaaatacattttatttggggaggaacatgtggctgagcagacttttgtataatattacttcaaagatatgtaatcacaaacaaaaaaaactattttttataatgtcatttgagagagtttcatcagtacagttggtggacgttaattgtttgaatttgatagtctttgaatttaatcaagaaactacctggaaccagtgaaaaggaaagctggacttaaataatcttagaattaattgataaatgtctcttttaaaatctactgtatttattataatttacacccttgaaggtgatctcttgttttgtgttgtaaatatattgtttgtatgtttcccttcttgccttctgttataagtctcttcctttctcaaataaagttttttttaaaagaaaaaaaaaaaaaaaaaaaa

SEQ ID NO: 18 - DNA, 2697 bp, Homo sapiens
  source 1..2697: mol_type = other DNA
acttgtccgtcacgtgcggccgcccggcctctcggccttgccgcgcgcctggcggggttgggggggcggggaccaagatctgctgcgcctgcgttgtgggcgttctcggggagctgctgccgtagctgccgccgccgctaccaccgcgttcgggtgtagaatttggaatccctgcgccgcgttaacaatgaagcagagttcgaacgtgccggctttcctcagcaagctgtggacgcttgtggaggaaacccacactaacgagttcatcacctggagccagaatggccaaagttttctggtcttggatgagcaacgatttgcaaaagaaattcttcccaaatatttcaagcacaataatatggcaagctttgtgaggcaactgaatatgtatggtttccgtaaagtagtacatatcgactctggaattgtaaagcaagaaagagatggtcctgtagaatttcagcatccttacttcaaacaaggacaggatgacttgttggagaacattaaaaggaaggtttcatcttcaaaaccagaagaaaataaaattcgtcaggaagatttaacaaaaattataagtagtgctcagaaggttcagataaaacaggaaactattgagtccaggctttctgaattaaaaagtgagaatgagtccctttggaaggaggtgtcagaattacgagcaaagcatgcacaacagcaacaagttattcgaaagattgtccagtttattgttacattggttcaaaataaccaacttgtgagtttaaaacgtaaaaggcctctacttctaaacactaatggagcccaaaagaagaacctgtttcagcacatagtcaaagaaccaactgataatcatcatcataaagttccacacagtaggactgaaggtttaaagccaagggagaggatttcagatgacatcattatttatgatgttactgatgataatgcagatgaagaaaatatcccagttattccagaaactaatgaggatgttatatctgatccctccaactgtagccagtaccctgatattgtcatcgttgaagatgacaatgaagatgagtatgcacctgtcattcagagtggagagcagaatgaaccagccagagaatccctaagttcaggcagtgatggcagcagccctctcatgtctagtgctgtccagctaaatggctcatccagtctgacctcagaagatccagtgaccatgatggattccattttgaatgataacatcaatcttttgggaaaggttgagctgttggattatcttgacagtattgactgcagtttagaggacttccaggccatgctatcaggaagacaatttagcatagacccagatctcctggttgatcttttcactagttctgtgcagatgaatcccacagattacatcaataatacaaaatctgagaataaaggattagaaactaccaagaacaatgtagttcagccagtttcggaagagggaagaaaatctaaatccaaaccagataagcagcttatccagtataccgcctttccacttcttgcattcctcgatgggaaccctgcttcttctgttgaacaggcgagtacaacagcatcatcagaagttttgtcctctgtagataaacccatagaagttgatgagcttctggatagcagcctagacccagaaccaacccaaagtaagcttgttcgcctggagccattgactgaagctgaagctagtgaagctacactgttttatttatgtgaacttgctcctgcacctctggatagtgatatgccacttttagatagctaaatccccaggaagtggactttacatgtatatattcatcaaaatgatgaactatttattttaaagtatcatttggtactttttttgtaaattgctttgttttgtttaatcagatactgtggaataaaagcaccttttgcttttctcactaaccacacactcttgcagagctttcaggtgttactcagctgcatagttacgcagatgtaatgcacattattggcgtatctttaagttggattcaaatggccatttttctccaattttggtaaattggatatcttttttttacaaatacgaccattaacctcagttaaatttttgtttgttttcctgtttgatgctgtctatttgcattgagtgtaagtcatttgaactaatggtataactcctaaagctttctctgctccagttatttttattaaatatttttcacttggcttatttttaaaactgggaacataaagtgcctgtatcttgtaaaacttcatttgtttcttttggttcagagaagttcatttatgttcaaagacgtttattcatgttcaacaggaaagacaaagtgtacgtgaatgctcgctgtctgatagggttccagctccatatatatagaaagatcgggggtgggatgggatggagtgagccccatccagttagttggactagttttaaataaaggttttccggtttgtgtttttttgaaccatactgtttagtaaaataaatacaatgaatgttgagtactagtgtctgttatgtgtcttctttagaggtgacactcacatgaaacaattttttcttctcataggaagcagtagctttaaactgtctgtggttcattattctcaatatgaatcataccaagatatttgtgcctcatctcgaaaatatattgtatattg

SEQ ID NO: 19 - DNA, 11427 bp, Homo sapiens
  source 1..11427: mol_type = other DNA
taccgggcggaggtgagcgcggcgccggctcctcctgcggcggactttgggtgcgacttgacgagcggtggttcgacaagtggccttgcgggccggatcgtcccagtggaagagttgtaaatttgcttctggccttcccctacggattatacctggccttcccctacggattatactcaacttactgtttagaaaatgtggcccacgagacgcctggttactatcaaaaggagcggggtcgacggtccccactttcccctgagcctcagcacctgcttgtttggaaggggtattgaatgtgacatccgtatccagcttcctgttgtgtcaaaacaacattgcaaaattgaaatccatgagcaggaggcaatattacataatttcagttccacaaatccaacacaagtaaatgggtctgttattgatgagcctgtacggctaaaacatggagatgtaataactattattgatcgttccttcaggtatgaaaatgaaagtcttcagaatggaaggaagtcaactgaatttccaagaaaaatacgtgaacaggagccagcacgtcgtgtctcaagatctagcttctcttctgaccctgatgagagtgagggaatacctttgaaaagaaggcgtgtgtcctttggtgggcacctaagacctgaactatttgatgaaaacttgcctcctaatacgcctctcaaaaggggagaagccccaaccaaaagaaagtctctggtaatgcacactccacctgtcctgaagaaaatcatcaaggaacagcctcaaccatcaggaaaacaagagtcaggttcagaaatccatgtggaagtgaaggcacaaagcttggttataagccctccagctcctagtcctaggaaaactccagttgccagtgatcaacgccgtaggtcctgcaaaacagcccctgcttccagcagcaaatctcagacagaggttcctaagagaggagggagaaagagtggcaacctgccttcaaagagagtgtctatcagccgaagtcaacatgatattttacagatgatatgttccaaaagaagaagtggtgcttcggaagcaaatctgattgttgcaaaatcatgggcagatgtagtaaaacttggtgcaaaacaaacacaaactaaagtcataaaacatggtcctcaaaggtcaatgaacaaaaggcaaagaagacctgctactccaaagaagcctgtgggcgaagttcacagtcaatttagtacaggccacgcaaactctccttgtaccataataatagggaaagctcatactgaaaaagtacatgtgcctgctcgaccctacagagtgctcaacaacttcatttccaaccaaaaaatggactttaaggaagatctttcaggaatagctgaaatgttcaagaccccagtgaaggagcaaccgcagttgacaagcacatgtcacatcgctatttcaaattcagagaatttgcttggaaaacagtttcaaggaactgattcaggagaagaacctctgctccccacctcagagagttttggaggaaatgtgttcttcagtgcacagaatgcagcaaaacagccatctgataaatgctctgcaagccctcccttaagacggcagtgtattagagaaaatggaaacgtagcaaaaacgcccaggaacacctacaaaatgacttctctggagacaaaaacttcagatactgagacagagccttcaaaaacagtatccactgcaaacaggtcaggaaggtctacagagttcaggaatatacagaagctacctgtggaaagtaagagtgaagaaacaaatacagaaattgttgagtgcatcctaaaaagaggtcagaaggcaacactactacaacaaaggagagaaggagagatgaaggaaatagaaagaccttttgagacatataaggaaaatattgaattaaaagaaaacgatgaaaagatgaaagcaatgaagagatcaagaacttgggggcagaaatgtgcaccaatgtctgacctgacagacctcaagagcttgcctgatacagaactcatgaaagacacggcacgtggccagaatctcctccaaacccaagatcatgccaaggcaccaaagagtgagaaaggcaaaatcactaaaatgccctgccagtcattacaaccagaaccaataaacaccccaacacacacaaaacaacagttgaaggcatccctggggaaagtaggtgtgaaagaagagctcctagcagtcggcaagttcacacggacgtcaggggagaccacgcacacgcacagagagccagcaggagatggcaagagcatcagaacgtttaaggagtctccaaagcagatcctggacccagcagcccgtgtaactggaatgaagaagtggccaagaacgcctaaggaagaggcccagtcactagaagacctggctggcttcaaagagctcttccagacaccaggtccctctgaggaatcaatgactgatgagaaaactaccaaaatagcctgcaaatctccaccaccagaatcagtggacactccaacaagcacaaagcaatggcctaagagaagtctcaggaaagcagatgtagaggaagaattcttagcactcaggaaactaacaccatcagcagggaaagccatgcttacgcccaaaccagcaggaggtgatgagaaagacattaaagcatttatgggaactccagtgcagaaactggacctggcaggaactttacctggcagcaaaagacagctacagactcctaaggaaaaggcccaggctctagaagacctggctggctttaaagagctcttccagactcctggtcacaccgaggaattagtggctgctggtaaaaccactaaaataccctgcgactctccacagtcagacccagtggacaccccaacaagcacaaagcaacgacccaagagaagtatcaggaaagcagatgtagagggagaactcttagcgtgcaggaatctaatgccatcagcaggcaaagccatgcacacgcctaaaccatcagtaggtgaagagaaagacatcatcatatttgtgggaactccagtgcagaaactggacctgacagagaacttaaccggcagcaagagacggccacaaactcctaaggaagaggcccaggctctggaagacctgactggctttaaagagctcttccagacccctggtcatactgaagaagcagtggctgctggcaaaactactaaaatgccctgcgaatcttctccaccagaatcagcagacaccccaacaagcacaagaaggcagcccaagacacctttggagaaaagggacgtacagaaggagctctcagccctgaagaagctcacacagacatcaggggaaaccacacacacagataaagtaccaggaggtgaggataaaagcatcaacgcgtttagggaaactgcaaaacagaaactggacccagcagcaagtgtaactggtagcaagaggcacccaaaaactaaggaaaaggcccaacccctagaagacctggctggcttgaaagagctcttccagacaccagtatgcactgacaagcccacgactcacgagaaaactaccaaaatagcctgcagatcacaaccagacccagtggacacaccaacaagctccaagccacagtccaagagaagtctcaggaaagtggacgtagaagaagaattcttcgcactcaggaaacgaacaccatcagcaggcaaagccatgcacacacccaaaccagcagtaagtggtgagaaaaacatctacgcatttatgggaactccagtgcagaaactggacctgacagagaacttaactggcagcaagagacggctacaaactcctaaggaaaaggcccaggctctagaagacctggctggctttaaagagctcttccagacacgaggtcacactgaggaatcaatgactaacgataaaactgccaaagtagcctgcaaatcttcacaaccagacccagacaaaaacccagcaagctccaagcgacggctcaagacatccctggggaaagtgggcgtgaaagaagagctcctagcagttggcaagctcacacagacatcaggagagactacacacacacacacagagccaacaggagatggtaagagcatgaaagcatttatggagtctccaaagcagatcttagactcagcagcaagtctaactggcagcaagaggcagctgagaactcctaagggaaagtctgaagtccctgaagacctggccggcttcatcgagctcttccagacaccaagtcacactaaggaatcaatgactaacgaaaaaactaccaaagtatcctacagagcttcacagccagacctagtggacaccccaacaagctccaagccacagcccaagagaagtctcaggaaagcagacactgaagaagaatttttagcatttaggaaacaaacgccatcagcaggcaaagccatgcacacacccaaaccagcagtaggtgaagagaaagacatcaacacgtttttgggaactccagtgcagaaactggaccagccaggaaatttacctggcagcaatagacggctacaaactcgtaaggaaaaggcccaggctctagaagaactgactggcttcagagagcttttccagacaccatgcactgataaccccacgactgatgagaaaactaccaaaaaaatactctgcaaatctccgcaatcagacccagcggacaccccaacaaacacaaagcaacggcccaagagaagcctcaagaaagcagacgtagaggaagaatttttagcattcaggaaactaacaccatcagcaggcaaagccatgcacacgcctaaagcagcagtaggtgaagagaaagacatcaacacatttgtggggactccagtggagaaactggacctgctaggaaatttacctggcagcaagagacggccacaaactcctaaagaaaaggccaaggctctagaagatctggctggcttcaaagagctcttccagacaccaggtcacactgaggaatcaatgaccgatgacaaaatcacagaagtatcctgcaaatctccacaaccagacccagtcaaaaccccaacaagctccaagcaacgactcaagatatccttggggaaagtaggtgtgaaagaagaggtcctaccagtcggcaagctcacacagacgtcagggaagaccacacagacacacagagagacagcaggagatggaaagagcatcaaagcgtttaaggaatctgcaaagcagatgctggacccagcaaactatggaactgggatggagaggtggccaagaacacctaaggaagaggcccaatcactagaagacctggccggcttcaaagagctcttccagacaccagaccacactgaggaatcaacaactgatgacaaaactaccaaaatagcctgcaaatctccaccaccagaatcaatggacactccaacaagcacaaggaggcggcccaaaacacctttggggaaaagggatatagtggaagagctctcagccctgaagcagctcacacagaccacacacacagacaaagtaccaggagatgaggataaaggcatcaacgtgttcagggaaactgcaaaacagaaactggacccagcagcaagtgtaactggtagcaagaggcagccaagaactcctaagggaaaagcccaacccctagaagacttggctggcttgaaagagctcttccagacaccaatatgcactgacaagcccacgactcatgagaaaactaccaaaatagcctgcagatctccacaaccagacccagtgggtaccccaacaatcttcaagccacagtccaagagaagtctcaggaaagcagacgtagaggaagaatccttagcactcaggaaacgaacaccatcagtagggaaagctatggacacacccaaaccagcaggaggtgatgagaaagacatgaaagcatttatgggaactccagtgcagaaattggacctgccaggaaatttacctggcagcaaaagatggccacaaactcctaaggaaaaggcccaggctctagaagacctggctggcttcaaagagctcttccagacaccaggcactgacaagcccacgactgatgagaaaactaccaaaatagcctgcaaatctccacaaccagacccagtggacaccccagcaagcacaaagcaacggcccaagagaaacctcaggaaagcagacgtagaggaagaatttttagcactcaggaaacgaacaccatcagcaggcaaagccatggacacaccaaaaccagcagtaagtgatgagaaaaatatcaacacatttgtggaaactccagtgcagaaactggacctgctaggaaatttacctggcagcaagagacagccacagactcctaaggaaaaggctgaggctctagaggacctggttggcttcaaagaactcttccagacaccaggtcacactgaggaatcaatgactgatgacaaaatcacagaagtatcctgtaaatctccacagccagagtcattcaaaacctcaagaagctccaagcaaaggctcaagatacccctggtgaaagtggacatgaaagaagagcccctagcagtcagcaagctcacacggacatcaggggagactacgcaaacacacacagagccaacaggagatagtaagagcatcaaagcgtttaaggagtctccaaagcagatcctggacccagcagcaagtgtaactggtagcaggaggcagctgagaactcgtaaggaaaaggcccgtgctctagaagacctggttgacttcaaagagctcttctcagcaccaggtcacactgaagagtcaatgactattgacaaaaacacaaaaattccctgcaaatctcccccaccagaactaacagacactgccacgagcacaaagagatgccccaagacacgtcccaggaaagaagtaaaagaggagctctcagcagttgagaggctcacgcaaacatcagggcaaagcacacacacacacaaagaaccagcaagcggtgatgagggcatcaaagtattgaagcaacgtgcaaagaagaaaccaaacccagtagaagaggaacccagcaggagaaggccaagagcacctaaggaaaaggcccaacccctggaagacctggccggcttcacagagctctctgaaacatcaggtcacactcaggaatcactgactgctggcaaagccactaaaataccctgcgaatctcccccactagaagtggtagacaccacagcaagcacaaagaggcatctcaggacacgtgtgcagaaggtacaagtaaaagaagagccttcagcagtcaagttcacacaaacatcaggggaaaccacggatgcagacaaagaaccagcaggtgaagataaaggcatcaaagcattgaaggaatctgcaaaacagacaccggctccagcagcaagtgtaactggcagcaggagacggccaagagcacccagggaaagtgcccaagccatagaagacctagctggcttcaaagacccagcagcaggtcacactgaagaatcaatgactgatgacaaaaccactaaaataccctgcaaatcatcaccagaactagaagacaccgcaacaagctcaaagagacggcccaggacacgtgcccagaaagtagaagtgaaggaggagctgttagcagttggcaagctcacacaaacctcaggggagaccacgcacaccgacaaagagccggtaggtgagggcaaaggcacgaaagcatttaagcaacctgcaaagcggaagctggacgcagaagatgtaattggcagcaggagacagccaagagcacctaaggaaaaggcccaacccctggaagatctggccagcttccaagagctctctcaaacaccaggccacactgaggaactggcaaatggtgctgctgatagctttacaagcgctccaaagcaaacacctgacagtggaaaacctctaaaaatatccagaagagttcttcgggcccctaaagtagaacccgtgggagacgtggtaagcaccagagaccctgtaaaatcacaaagcaaaagcaacacttccctgcccccactgcccttcaagaggggaggtggcaaagatggaagcgtcacgggaaccaagaggctgcgctgcatgccagcaccagaggaaattgtggaggagctgccagccagcaagaagcagagggttgctcccagggcaagaggcaaatcatccgaacccgtggtcatcatgaagagaagtttgaggacttctgcaaaaagaattgaacctgcggaagagctgaacagcaacgacatgaaaaccaacaaagaggaacacaaattacaagactcggtccctgaaaataagggaatatccctgcgctccagacgccaaaataagactgaggcagaacagcaaataactgaggtctttgtattagcagaaagaatagaaataaacagaaatgaaaagaagcccatgaagacctccccagagatggacattcagaatccagatgatggagcccggaaacccatacctagagacaaagtcactgagaacaaaaggtgcttgaggtctgctagacagaatgagagctcccagcctaaggtggcagaggagagcggagggcagaagagtgcgaaggttctcatgcagaatcagaaagggaaaggagaagcaggaaattcagactccatgtgcctgagatcaagaaagacaaaaagccagcctgcagcaagcactttggagagcaaatctgtgcagagagtaacgcggagtgtcaagaggtgtgcagaaaatccaaagaaggctgaggacaatgtgtgtgtcaagaaaataagaaccagaagtcatagggacagtgaagatatttgacagaaaaatcgaactgggaaaaatataataaagttagttttgtgataagttctagtgcagtttttgtcataaattacaagtgaattctgtaagtaaggctgtcagtctgcttaagggaagaaaactttggatttgctgggtctgaatcggcttcataaactccactgggagcactgctgggctcctggactgagaatagttgaacaccgggggctttgtgaaggagtctgggccaaggtttgccctcagctttgcagaatgaagccttgaggtctgtcaccacccacagccaccctacagcagccttaactgtgacacttgccacactgtgtcgtcgtttgtttgcctatgtcctccagggcacggtggcaggaacaactatcctcgtctgtcccaacactgagcaggcactcggtaaacacgaatgaatggatgagcgcacggatgaatggagcttacaagatctgtctttccaatggccgggggcatttggtccccaaattaaggctattggacatctgcacaggacagtcctatttttgatgtcctttcctttctgaaaataaagttttgtgctttggagaatgactcgtgagcacatctttagggaccaagagtgactttctgtaaggagtgactcgtggcttgccttggtctcttgggaatacttttctaactagggttgctctcacctgagacattctccacccgcggaatctcagggtcccaggctgtgggccatcacgacctcaaactggctcctaatctccagctttcctgtcattgaaagcttcggaagtttactggctctgctcccgcctgttttctttctgactctatctggcagcccgatgccacccagtacaggaagtgacaccagtactctgtaaagcatcatcatccttggagagactgagcactcagcaccttcagccacgatttcaggatcgcttccttgtgagccgctgcctccgaaatctcctttgaagcccagacatctttctccagcttcagacttgtagatataactcgttcatcttcatttactttccactttgccccctgtcctctctgtgttccccaaatcagagaatagcccgccatcccccaggtcacctgtctggattcctccccattcacccaccttgccaggtgcaggtgaggatggtgcaccagacagggtagctgtcccccaaaatgtgccctgtgcgggcagtgccctgtctccacgtttgtttccccagtgtctggcggggagccaggtgacatcataaatacttgctgaatgaatgcagaaatcagcggtactgacttgtactatattggctgccatgatagggttctcacagcgtcatccatgatcgtaagggagaatgacattctgcttgagggagggaatagaaaggggcagggaggggacatctgagggcttcacagggctgcaaagggtacagggattgcaccagggcagaacaggggagggtgttcaaggaagagtggctcttagcagaggcactttggaaggtgtgaggcataaatgcttccttctacgtaggccaacctcaaaactttcagtaggaatgttgctatgatcaagttgttctaacactttagacttagtagtaattatgaacctcacatagaaaaatttcatccagccatatgcctgtggagtggaatattctgtttagtagaaaaatcctttagagttcagctctaaccagaaatcttgctgaagtatgtcagcaccttttctcaccctggtaagtacagtatttcaagagcacgctaagggtggttttcattttacagggctgttgatgatgggttaaaaatgttcatttaagggctacccccgtgtttaatagatgaacaccacttctacacaaccctccttggtactgggggagggagagatctgacaaatactgcccattcccctaggctgactggatttgagaacaaatacccacccatttccaccatggtatggtaacttctctgagcttcagtttccaagtgaatttccatgtaataggacattcccattaaatacaagctgtttttactttttcgcctcccagggcctgtgggatctggtcccccagcctctcttgggctttcttacactaactctgtacctaccatctcctgcctcccttaggcaggcacctccaaccaccacacactccctgctgttttccctgcctggaactttccctcctgccccaccaagatcatttcatccagtcctgagctcagcttaagggaggcttcttgcctgtgggttccctcacccccatgcctgtcctccaggctggggcaggttcttagtttgcctggaattgttctgtacctctttgtagcacgtagtgttgtggaaactaagccactaattgagtttctggctcccctcctggggttgtaagttttgttcattcatgagggccgactgcatttcctggttactctatcccagtgaccagccacaggagatgtccaataaagtatgtgatgaaatggtcttaaaaaaaaaaaaaa

SEQ ID NO: 20 - DNA, 5765 bp, Homo sapiens
  source 1..5765: mol_type = other DNA
tcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgactatgagtgtgtatttattcatgaaatttgaactgtttgccccgaaatggatatggaatactttataagccatagacactatagtataccagtgaatcttttatgcagcttgttagaagtatcctttatttctaaaaggtgctgtggatattatgtaaaggcgtgtttgcttaaacttaaaaccatatttagaagtagatgcaaaacaaatctgcctttatgacaaaaaaataggataacattatttatttatttccttttatcaaagaaggtaattgatacacaacaggtgacttggttttaggcccaaaggtagcagcagcaacattaataatggaaataattgaatagttagttatgtatgttaatgccagtcaccagcaggctatttcaaggtcagaagtaatgactccatacatattatttatttctataactacatttaaatcattaccagg

SEQ ID NO: 21 - DNA, 2202 bp, Homo sapiens
  source 1..2202: mol_type = other DNA
cgtaaagagaggccgggagctgcccctaaccgaggcagcagcggacgtgagcgataatggcggatatggaggatctcttcgggagcgacgccgacagcgaagctgagcgtaaagattctgattctggatctgactcagattctgatcaagagaatgctgcctctggcagtaatgcctctggaagtgaaagtgatcaggatgaaagaggtgattcaggacaaccaagtaataaggaactgtttggagatgacagtgaggacgagggagcttcacatcatagtggtagtgataatcactctgaaagatcagacaatagatcagaagcttctgagcgttctgaccatgaggacaatgacccctcagatgtagatcagcacagtggatcagaagcccctaatgatgatgaagacgaaggtcatagatcggatggagggagccatcattcagaagcagaaggttctgaaaaagcacattcagatgatgaaaaatggggcagagaagataaaagtgaccagtcagatgatgaaaagatacaaaattctgatgatgaggagagggcacaaggatctgatgaagataagctgcagaattctgacgatgatgagaaaatgcagaacacagatgatgaggagaggcctcagctttccgatgatgagagacaacagctatctgaggaggaaaaggctaattctgatgatgaacggccggtagcttctgataatgatgatgagaaacagaattctgatgatgaagaacaaccacagctgtctgatgaagagaaaatgcaaaattctgatgatgaaaggccacaggcctcagatgaagaacacaggcattcagatgatgaagaggaacaggatcataaatcagaatctgcaagaggcagtgatagtgaagatgaagttttacgaatgaaacgcaagaatgcgattgcatctgattcagaagcggatagtgacactgaggtgccaaaagataatagtggaaccatggatttatttggaggtgcagatgatatctcttcagggagtgatggagaagacaaaccacctactccaggacagcctgttgatgaaaatggattgcctcaggatcaacaggaagaggagccaattcctgagaccagaatagaagtagaaatacccaaagtaaacactgatttaggaaacgacttatattttgttaaactgcccaactttctcagtgtagagcccagaccttttgatcctcagtattatgaagatgaatttgaagatgaagaaatgctggatgaagaaggtagaaccaggttaaaattaaaggtagaaaatactataagatggaggatacgccgagatgaagaaggaaatgaaattaaagaaagcaatgctcggatagtcaagtggtcagatggaagcatgtccctgcatttaggcaatgaagtgtttgatgtgtacaaagccccactgcagggcgaccacaatcatctttttataagacaaggtactggtctacagggacaagcagtctttaaaacgaaactcaccttcagacctcactctacggacagtgccacacatagaaagatgactctgtcacttgcagataggtgttcaaagacacagaagattagaatcttgccaatggctggtcgtgatcctgaatgccaacgcacagaaatgattaagaaagaagaagaacgtttgagggcttccatacgtagggaatctcagcagcgccgaatgagagagaaacagcaccagcgggggctgagcgccagttacctggaacctgatcgatacgatgaggaggaggaaggcgaggagtccatcagcttggctgccattaaaaaccgatataaagggggcattcgagaggaacgagccagaatctattcatcagacagtgatgagggatcagaagaagataaagctcaaagattactcaaagcaaagaaacttaccagtgatgaggaaggtgaaccttccggaaagagaaaagcagaagatgatgataaagcaaataaaaagcataagaagtatgtgatcagcgatgaagaggaagaagatgatgattgaagtatgaaatatgaaaacattttatatattttattgtacagttataaatatgtaaacatgagttattttgattgaaatgaatcgatttgcttttgtgtaattttaattgtaataaaacaatttaaaagcaaaaaaaaaaaaaaaa

SEQ ID NO: 22 - DNA, 2047 bp, Homo sapiens
  source 1..2047: mol_type = other DNA
ttgattatggaacattctaaaacttagacaagacgattgtgattggctgaagggcatacgccctcctccagggtgacgtgtctgcctatggatatcagttgccagagaaacctggctttactatggcggttggaggaacggcagtgatcacacgtcggctgctgggaagatctggattctcgtttcaggtcaccatcagaaaagctaagtttgctgtatagtgaggatcaggagatctgatcctgattgcagaaccttccctgattacagaatcttgggattgttgagaggattacatgtaaagtaccaggacagtgcatggcacatatgatttcacaaaagttcatcttcattgcagatacctgcctttctttctaggttgtatctcccacttcacccttctagaccatcccagaagatctataagatttcatctgggaaatcactaggagttcttggaagggaaagaaggaagattgttggttggaataaaaacagggttgaatgagttccagaaagcagggttctcaacctcgtggacagcaatctgcagaagaagagaacttcaaaaaaccaactagaagcaacatgcagagaagtaaaatgagaggggcctcctcaggaaagaagacagctggtccacagcagaaaaatcttgaaccagctctcccaggaagatggggtggtcgctctgcagagaaccccccttcaggatccgtgaggaagaccagaaagaacaagcagaagactcctggaaacggagatggtggcagtaccagcgaagcacctcagccccctcggaagaaaagggcccgggcagaccccactgttgaaagtgaggaggcgtttaagaatagaatggaggttaaagtgaagattcctgaagaattaaaaccatggcttgttgaggactgggacttagttaccaggcagaagcagctgtttcaactccctgccaagaaaaatgtagatgcaattctggaggagtatgcaaattgcaagaaatcgcagggaaatgttgataataaggaatatgcggttaatgaagttgtggcaggaataaaagaatatttcaatgtgatgttgggcactcagctgctctacaaatttgagaggccccagtatgctgaaatcctcttggctcaccctgatgctccaatgtcccaggtttatggagcaccacacctactgagattatttgtaagaattggagcaatgttggcctatacgccccttgatgagaaaagccttgcattattgttgggctatttgcatgatttcctaaaatatctggcaaagaattctgcatctctctttactgccagtgattacaaagtggcttctgctgagtaccaccgcaaagccctgtgagcgtctacagacagctcaccatttttgtcctgtatctgtaaacactttttgttcttagtctttttcttgtaaaattgatgttctttaaaatcgttaatgtataacagggcttatgtttcagtttgttttccgttctgttttaaacagaaaataaaaggagtgtaagctccttttctcatttcaaagttgctaccagtgtatgcagtaattagaacaaagaagaaacattcagtagaacattttattgcctagttgacaacattgcttgaatgctggtggttcctatccctttgacactacacaattttctaatatgtgttaatgctatgtgacaaaacgccctgattcctagtgccaaaggttcaacttaatgtatatacctgaaaacccatgcatttgtgctctttttttttttttatggtgcttgaagtaaaacagcccatcctctgcaagtccatctatgttgttcttaggcattctatctttgctcaaattgttgaaggatggtgatttgtttcatggtttttgtatttgagtctaatgcacgttctaacatgatagaggcaatgcattattgtgtagccacggttttctggaaaagttgatattttaggaattgtatttcagatcttaaataaaatttgtttctaaatttcaaagcaaaaaaaaaaaaaaa

SEQ ID NO: 23 - DNA, 4451 bp, Homo sapiens
  source 1..4451: mol_type = other DNA
aaaagatatggtggggtgcttaacagaggaggttagacaccggcgggaaccagaggagcccaagcgcggcgcctgggcctcggggctgcaggagtcctcggtgggggtatggaggtcgccggggaaggaggacggttcagttgctaggcaacccggcctggacccgcctctcgctcgcgttgctgggagactacaaggccgggaggagggcggcgaaagggccctacgtgctgacgctaattgtatatgagcgcgagcggcgggctcttgggtcttttttagcgccatctgctcgcggcgccgcctcctgctcctcccgctgctgctgccgctgccgccctgagtcactgcctgcgcagctccggccgcctggctccccatactagtcgccgatatttggagttcttacaacatggcagacattgacaacaaagaacagtctgaacttgatcaagatttggatgatgttgaagaagtagaagaagaggaaactggtgaagaaacaaaactcaaagcacgtcagctaactgttcagatgatgcaaaatcctcagattcttgcagcccttcaagaaagacttgatggtctggtagaaacaccaacaggatacattgaaagcctgcctagggtagttaaaagacgagtgaatgctctcaaaaacctgcaagttaaatgtgcacagatagaagccaaattctatgaggaagttcacgatcttgaaaggaagtatgctgttctctatcagcctctatttgataagcgatttgaaattattaatgcaatttatgaacctacggaagaagaatgtgaatggaaaccagatgaagaagatgagatttcggaggaattgaaagaaaaggccaagattgaagatgagaaaaaagatgaagaaaaagaagaccccaaaggaattcctgaattttggttaactgtttttaagaatgttgacttgctcagtgatatggttcaggaacacgatgaacctattctgaagcacttgaaagatattaaagtgaagttctcagatgctggccagcctatgagttttgtcttagaatttcactttgaacccaatgaatattttacaaatgaagtgctgacaaagacatacaggatgaggtcagaaccagatgattctgatcccttttcttttgatggaccagaaattatgggttgtacagggtgccagatagattggaaaaaaggaaagaatgtcactttgaaaactattaagaagaagcagaaacacaagggacgtgggacagttcgtactgtgactaaaacagtttccaatgactctttctttaacttttttgcccctcctgaagttcctgagagtggagatctggatgatgatgctgaagctatccttgctgcagacttcgaaattggtcactttttacgtgagcgtataatcccaagatcagtgttatattttactggagaagctattgaagatgatgatgatgattatgatgaagaaggtgaagaagcggatgaggaaggggaagaagaaggagatgaggaaaatgatccagactatgacccaaagaaggatcaaaacccagcagagtgcaagcagcagtgaagcaggatgtatgtggccttgaggataacctgcactggtctaccttctgcttccctggaaaggatgaatttacatcatttgacaagcctattttcaagttatttgttgtttgtttgcttgtttttgtttttgcagctaaaataaaaatttcaaatacaattttagttcttacaagataatgtcttaattttgtaccaattcaggtagaagtagaggcctaccttgaattaagggttatactcagtttttaacacattgttgaagaaaaggtaccagctttggaacgagatgctatactaataagcaagtgtaaaaaaaaaaaaaaaagaggaagaaaatcttaagtgattgatgctgttttcttttaaaaaaaaaaaaaaaaattcattttctttgggttagagctagagagaaggccccaagcttctatggtttcttctaattcttattgcttaaagtatgagtatgtcacttacccgtgcttctgtttactgtgtaattaaaatgggtagtactgtttacctaactacctcatggatgtgttaaggcatattgagttaaatctcatataatgtttctcaatcttgttaaaagctcaaaattttgggcctatttgtaatgccagtgtgacactaagcattttgttcacaccacgctttgataactaaactggaaaacaaaggtgttaagtacctctgttctggatctgggcagtcagcactctttttagatctttgtgtggctcctatttttatagaagtggagggatgcactatttcacaaggtccaagatttgttttcagatatttttgatgactgtattgtaaatactacagggatagcactatagtattgtagtcatgagacttaaagtggaaataagactatttttgacaaaagatgccattaaatttcagactgtagagccacatttacaatacctcaggctaattactgttaattttggggttgaacttttttttgacagtgagggtggattattggattgtcattagaggaaggtctagatttcctgctcttaataaaattacattgaattgatttttagaggtaatgaaaacttcctttctgagaagttagtgttaaggtcttggaatgtgaacacattgtttgtagtgctatccattcctctcctgagattttaacttactactggaaatccttaaccaattataatagctttttttctttattttcaaaatgatttcctttgctttgattagacactatgtgctttttttttttaaccatagttcatcgaaatgcagctttttctgaacttcaaagatagaatcccatttttaatgaactgaagtagcaaaatcatctttttcattctttaggaaatagctattgccaaagtgaaggtgtagataatacctagtcttgttacataaaggggatgtggtttgcagaagaattttctttataaaattgaagttttaagggacgtcagtgtttatgccatttttccagttccaaaatgattccattccattctagaaatttgaagtatgtaacctgaaatccttaataaaatttggatttaattttataaaatgtactggtgatattttgggtgtttttttttaaatgaatgtatatactttttttttgaagagtggagagtagtgatgtctagagggagctattttgtgctgaggccactatgttctgtaaatatataattttaagagcaacctcacaatccctgctaagtggagtttattatttgaagactaaaatggaattccatagttcctgataggttatattctgggttattattctgagttatctacaaacatttttgagatttgtctttacactctgattgtagtttccagcagcccatgcacactgccaagtaagtctcattttttcctgttagaaatggtgaaatatcatataatcacttataaagaaaactgatatgaaaaaattttagagttgtttgctttatggtcactcaagtagggtaagtgttccacaaattccacaagttgatagtttaacatggatgtctgaaagccacatatataatttcttaggattcttaaattagtaaatctagcttactgaagcagtattagcatcactattttagattgcaaaaataccttaattgtgtggaactggcttgtagagtggtacttaagaaaaatgggattctacctctatttctgttttagcacacttaatcaggaaaggatatattaactttcataaaaatatttttgttgtgtgaataggttaatgatatggtaaggcccctaaaataactgaattaattgtttattgtaattgtaggccattcccattattaaaaataaagacaaaacttgaagtaactgaaaatcttatcgtgctatgtagaaatattgaactaatattcaaatatttgaatgctttggtttcagggattggtttaaaattggagtccttttttatgggttagtcttacaaaaatttaagcctttatatttttgactttaaatcaaaacaaatgttattttaaatgtacagaatagattggtagtgcagaagagtgtaagttcttcataggagctttagaaaagagaaatatgtgctaattcagtttttttttaatctgcactgtacatatatacttggtaattatgagcttgattttgtttttggaaatatgtgttcataatttaggtaatttgctacttaaagcactaagtctctgatacctgaaaagtacatgtaaatggtgatggtgaaataatactgcagttaacttaatagatgtatactggtgatttttgtatgctggattaaaactccagatattaaaatataacctggataaaaagcc

SEQ ID NO: 24 - DNA, 1530 bp, Homo sapiens
  source 1..1530: mol_type = other DNA
ggcattcagagagtagatgccagtcctgggaaaggcaggggaggagaggagagccacggctgacgcttggggacagaaggaggagcctgaggaggagacaggacagagcgtctggagaggcaggaggacaccgagttccccgtgttggcctccaggtcctgtgcttgcggagccgtccggcggctgggatcgagccccgacaatgggcaacgcgcaggagcggccgtcagagactatcgaccgcgagcggaaacgcctggtcgagacgctgcaggcggactcgggactgctgttggacgcgctgctggcgcggggcgtgctcaccgggccagagtacgaggcattggatgcactgcctgatgccgagcgcagggtgcgccgcctactgctgctggtgcagggcaagggcgaggccgcctgccaggagctgctacgctgtgcccagcgtaccgcgggcgcgccggaccccgcttgggactggcagcacgctaccgggaccgcagctatgaccctccatgcccaggccactggacgccggaggcacccggctcggggaccacatgccccgggttgcccagagcttcagaccctgacgaggccgggggccctgagggctccgaggcggtgcaatccgggaccccggaggagccagagccagagctggaagctgaggcctctaaagaggctgaaccggagccggagccagagccagagctggaacccgaggctgaagcagaaccagagccggaactggagccagaaccggacccagagcccgagcccgacttcgaggaaagggacgagtccgaagattcctgaaggccagagctctgacaggcggtgccccgcccatgctggataggacctgggatgctgctggagctgaatcggatgccaccaaggctcggtccagcccagtaccgctggaagtgaataaactccggagggtcggacgggacctgggctctctccacgattctggctgtttgcccaggaacttagggtgggtacctctgagtcccagggacctgggcaggcccaagcccaccacgagcatcatccagtcctcagccctaatctgcccttaggagtccaggctgcaccctggagatcccaaacctagccccctagtgggacaaggacctgaccctcctgcccgcatacacaacccatttcccctggtgagccacttggcagcatatgtaggtaccagctcaaccccacgcaagttcctgagctgaacatggagcaaggggagggtgacttctctccacatagggagggcttagagctcacagccttgggaagtgagactagaagaggggagcagaaagggaccttgagtagacaaaggccacacacatcattgtcattactgttttaattgtctggcttctctctggactgggagctcagtgaggattctgaccagtgacttacacaaaaggcgctctatacatattataatatattcgcttactaaatgaataaggactttccaaaaaaaaaaaaaaaaaaaaaaaaaaaaaa

SEQ ID NO: 25 - DNA, 2410 bp, Homo sapiens
  source 1..2410: mol_type = other DNA
ggtgcagccttacgccgctgacgcatcgcgcccaagatggcggcgcggtcgtcgtcgggggtggcggcggcagagggggcggcggccctggcggcagcggagacggcagccgtgacggtggcagcggcggcgcgggacctgggcctgggggaatgaggcggccgcggcgggccagcggcggagccgtgtagcggagaagctccccctccctgcttcccttggccgagccgggggcgcgcgcgcacgcggccgtccagagcgggctccccacccctcgactcctgcgacccgcaccgcacccccacccgggcccggaggatgatgaagctcaagtcgaaccagacccgcacctacgacggcgacggctacaagaagcgggccgcatgcctgtgtttccgcagcgagagcgaggaggaggtgctactcgtgagcagtagtcgccatccagacagatggattgtccctggaggaggcatggagcccgaggaggagccaagtgtggcagcagttcgtgaagtctgtgaggaggctggagtaaaagggacattgggaagattagttggaatttttgagaaccaggagaggaagcacaggacgtatgtctatgtgctcattgtcactgaagtgctggaagactgggaagattcagttaacattggaaggaagagggaatggtttaaaatagaagacgccataaaagtgctgcagtatcacaaacccgtgcaggcatcatattttgaaacattgaggcaaggctactcagccaacaatggcaccccagtcgtggccaccacatactcggtttctgctcagagctcgatgtcaggcatcagatgactgaagacttcctgtaagagaaatggaaattggaaactagactgaagtgcaaatcttccctctcaccctggctctttccacttctcacaggcctcctctttcaaataaggcatggtgggcagcaaagaaagggtgtattgataatgttgctgtttggtgttaagtgatggggctttttcttctgtttttattgagggtgggggttgggtgtgtaatttgtaagtacttttgtgcatgatctgtccctccctcttcccacccctgcagtcctctgaagagaggccaacagccttcccctgccttggattctgaagtgttcctgtttgtcttatcctggccctggccagacgttttctttgatttttaattttttttttttattaaaagataccagtatgagatgaaaacttccaataatttgtcctataatgtgctgtacagttcagtagagtggtcactttcactgcagtatacatttatctacacattatatatcggacatataatatgtaaataaatgacttctagaaagagaaatttgtttaatttttcaaggtttttttctcttttaatttgggcatttctagaattgagagcctcacaattaacatacctttttgttttcgatgctagtggctgggcaggttgccctgtcctttctctatttcccagtcattgactgtagatatgggaagagtttagctaccttcatagtgctcccaggactcatggcctttccttctttaagctgtatttccctgcccagaaagaaacaggaagaaacctttttttatttttttatttttttttaaccaagcaaggagcaaatggcctcagcccagatctgtaaaaacaatgatagaaattgaattctgccccacatgttgacagtagagttggaactggattcttgggattacttatctaaaaaactggagcatcaggtccatttctgttctgctggtttggaatcttttccgtaatgctatttattgccaacaatggcctctctttgtgtccatatatgccttacaccgtgctgacctgggtatcatccatgtgctctgaagcatccaactttactttgcaggtgcatcaatgtagtcctgtccctgaactgagtaaccgtgttcctgaaaagtacactagggaaattcacctgcttgcttgtctttgtattggcatggcacttgtgattgcaccatggagcatgctcagagctattaaattggtctcccatctcccaccaggatatgaaaggtccatatgggaggccacgtaatcacttattacagtggttacataatacactggctcactgcagactctcttgttttttgatacagtttcgtgctggcttcatttgccaattgtgttgtttagttcggaagtaagagggtcttgagattgaggggtagggagggctacactgactgatccgtggcttaagacaggagattatctctgtactccagtggcatctccttagccaagatgtgaaattaaaatcatagttcgcctcatttaaaaattctaataaagcactcaaactttgaaaaaaaaaaaaaaaaaa

SEQ ID NO: 26 - DNA, 1947 bp, Homo sapiens
  source 1..1947: mol_type = other DNA
atgcagatgaggcactcgggggcggggcggcggcggcggcggcggcggtggcggccggggagggtcagttggaggcaggcgctcgctgaggcaaaaggaggcgctcggcccgcggcctgacagggacttagcccgcagagatcgaccccgcgcgcgtgaccccacacccacccactcatccatctatccactccctgcgccgcctcctcccaccctgagcagagccgccgaggatgataaacacccaggacagtagtattttgcctttgagtaactgtccccagctccagtgctgcaggcacattgttccagggcctctgtggtgctcctgatgcccctcacccactgtcgaagatccccggtgggcgagggggcggcagggatccttctctctcagctctaatatataaggacgagaagctcactgtgacccaggacctccctgtgaatgatggaaaacctcacatcgtccacttccagtatgaggtcaccgaggtgaaggtctcttcttgggatgcagtcctgtccagccagagcctgtttgtagaaatcccagatggattattagctgatgggagcaaagaaggattgttagcactgctagagtttgctgaagagaagatgaaagtgaactatgtcttcatctgcttcaggaagggccgagaagacagagctccactcctgaagaccttcagcttcttgggctttgagattgtacgtccaggccatccctgtgtcccctctcggccagatgtgatgttcatggtttatcccctggaccagaacttgtccgatgaggactaatagtcatagaggatgctttacccaagagccacagtgggggaagaggggaagttaggcagccctgggacagacgagagggctcctcgctgtctagggaaggacactgaggggctcagggtgagggttgcctattgtgttctcggagttgactcgttgaaattgttttccataaagaacagtataaacatattattcacatgtaatcaccaatagtaaatgaagatgtttatgaactggcattagaagctttctaaactgcgctgtgtgatgtgttctatctagcctaggggaggacattgcctagagggggagggactgtctgggttcaggggcatggcctggagggctggtgggcagcactgtcaggctcaggtttccctgctgttggctttctgttttggttattaagacttgtgtattttctttctttgcttcctgtcaccccaggggctcctgagtataggcttttcagtccctgggcagtgtccttgagttgttttttgacactcttacctgggcttctctgtgtgcatttgcgtctggcctggagtaagcaggtccgacccctccttctttacagcttagtgttattctggcatttggttaagctggcttaatctgtttaatgttatcagtacattttaaataggggcattgaaatttactcccaccaccagggcttttttgggggatgcctgggcctttaaaacactagccaaactctaattaattctcaaatcactgccaggagttcttgctcctggctgcaggcccaggccccaaggtctccttcttggggtcacaaacagcagtaaggaagaggaatatatagcaactcagggcctgggaattgtggggcaatccgttcttagggactggatacttctggctggctgagtatagtactagctgcctccccaccaggttccgagtagtgtctgagactctgctctgcagggcctagggtagcgctgggagtgtagaagtggcctgcccttaactgttttcactaaacagctttttctaaggggagagcaagggggagagatctagattgggtgagggggacggggatgtcagggaggcaagtgtgttgtgttactgtgtcaataaactgatttaaagttgtgaaaaaaaaaaaaa

SEQ ID NO: 27 - DNA, 1712 bp, Homo sapiens
  source 1..1712: mol_type = other DNA
atgctgggggaggggctggcggcctcgacggcagctgcggaactaggccgagggacaaaggctaagtttttccatggtttggactggatatcggtggaactctggtcaagctggtatattttgaacccaaagacatcactgctgaagaagaagaggaagaagtggaaagtcttaaaagcattcggaagtacctgacctccaatgtggcttatgggtctacaggcattcgggacgtgcacctcgagctgaaggacctgactctgtgtggacgcaaaggcaatctgcactttatacgctttcccactcatgacatgcctgcttttattcaaatgggcagagataaaaacttctcgagtctccacactgtcttttgtgccactggaggtggagcgtacaaatttgagcaggattttctcacaataggtgatcttcagctttgcaaactggatgaactagattgcttgatcaaaggaattttatacattgactcagtcggattcaatggacggtcacagtgctattactttgaaaaccctgctgattctgaaaagtgtcagaagttaccatttgatttgaaaaatccgtatcctctgcttctggtgaacattggctcaggggttagcatcttagcagtatattccaaagataattacaaacgggtcacaggtactagtcttggaggaggaactttttttggtctctgctgtcttcttactggctgtaccacttttgaagaagctcttgaaatggcatctcgtggagatagcaccaaagtggataaactagtacgagatatttatggaggggactatgagaggtttggactgccaggctgggctgtggcttcaagctttggaaacatgatgagcaaggagaagcgagaggctgtcagtaaagaggacctggccagagcgactttgatcaccatcaccaacaacattggctcaatagcaagaatgtgtgcccttaatgaaaacattaaccaggtggtatttgttggaaatttcttgagaattaatacgatcgccatgcggcttttggcatatgctttggattattggtccaaggggcagttgaaagcacttttttcggaacacgagggttattttggagctgttggagcactccttgagctgttgaagatcccgtgatcattacctggggaggggttcctgaaaccttccacaatgggatctgtggactttcatttttttaagagacttactcaatttcatgactgtactacctgaaacaaagtgagaaaggacaggtgtatttttctaagtcatcaagataaatccttaagaattcagtctaaattagcaaccaggaaggaaaaatatattaaaaacaacaaaaaagtggcacatgtccaggcagtgtgaggatttgctgtatataagttgcctgctttgtatttttgaaatctctgcatcactcattggaagtgcttctgaagagagctgctctgtgttcagttgactggttttgtgtcctgtttgaacttgctgaatgtaaggcaggctactatgcgttataatctaatcacaatttgtcaatatggtcttggcaatcatctgtgcattactctggtttgcattaagcctgtgtgtgaacttactgtaaaacatgttttatttcaaggttctgcaaaattaattgggcaggttaattgtgtacctgaaacttaacaagcagtttttggaagggca

SEQ ID NO: 28 - DNA, 7332 bp, Homo sapiens
  source 1..7332: mol_type = other DNA
ggtgaatgggctggtggtgctcgctgctgctgctgagaggaggaggaggatgaagagttgggcttgtttgtctcctacagtttctctcctgctgctctgattcccccctcccgattccggcccggggcctgtgtgtgtccctcctggaggaggaggaggatccagttcctccccccaaccccctcctccccacccccccttgcctggggaagaggaggaaagaaacagcccagagagagagagagagagagagtgagtgagagagagaggagaggagaggaggaggaggaggagggagaagggaacaacctaccatcttaacacactaatatctaaaaagtgcgagaggcccagagcagcagcagaagcagcagcagcagctccagcttcttccctccctccccatgaagaagagttccctcctcctcctcctcctgcttctcctgctcagagttcctgcctccagctgccaggggggacagccagccagcagcaggaggggggctagagagctgaaggagagccagtttccccaaaattgctgcagtgagaagaggagtttgttactttaaacagaggctgaagaaactatagaattagcagagaaagtggagaaggtagaggatggagttgcagactctacaggaggctcttaaagtggaaattcaggttcaccagaaactggttgctcaaatgaagcaggatccacagaatgctgacttaaagaaacagcttcatgaactccaagccaaaatcacagctttgagtgagaaacagaaaagagtagttgaacagctacggaagaacctgatagtaaagcaagaacaaccggacaagttccaaatacagccattgccacaatctgaaaacaaactacaaacagcacagcagcaaccactacagcaactacaacaacagcagcagtaccaccaccaccacgcccagcagtcagctgcagcctctcccaacctgactgcttcacagaagactgtaactacagcttctatgattaccacaaagacactacctctcgtcttgaaagcagcaactgcgaccatgcctgcctctgtggtgggccagagacctaccattgctatggtgaccgccatcaacagtcagaaggctgtgctcagcactgatgtgcagaacacaccagtcaacctccagacgtctagtaaggtcactgggcctggggcagaggctgtccaaattgtggcaaaaaacacagtcactctggttcaggcaacacctcctcagcccatcaaagtaccacagtttatcccccctcctagactcactccacgtccaaactttcttccacaggttcgacccaagcctgtggcccagaataacattcctattgccccagcaccacctcccatgctcgcagctcctcagcttatccagaggcccgtcatgctgaccaagttcacccccacaacccttcccacatcccagaattccatccaccccgtccgtgtcgtcaatgggcagactgcaaccatagccaaaacgttccccatggcccagctcaccagcattgtgatagctactccagggaccagactcgctggacctcaaactgtacagcttagcaagccaagtcttgaaaaacagacagttaaatctcacacagaaacagatgagaaacaaacagagagccgcaccatcaccccacctgctgcacccaaaccaaaacgggaggagaaccctcagaaacttgccttcatggtgtctctagggttggtaacacatgaccatctagaagaaatccaaagcaagaggcaagagcgaaaaagaagaacaacagcaaatccggtctacagtggagcagtctttgagccagagcgtaagaagagtgcagtgacatacctaaacagcacaatgcaccctgggacccggaagagaggtcgtcctccaaaatacaatgcagtgctggggtttggagcccttaccccaacatccccccaatccagtcatcctgactcccctgaaaatgaaaagacagagaccacattcactttccctgcacctgttcagcctgtgtccctgcccagccccacctccacagacggtgatattcatgaggatttttgcagcgtttgcagaaaaagtggccagttactgatgtgcgacacatgttcccgtgtatatcatttggactgcttagacccccctctgaaaacaattcccaagggcatgtggatctgtcccagatgtcaggaccagatgctgaagaaggaagaagcaattccatggcctggaactttagcaattgttcattcctatattgcctacaaagcagcaaaagaagaagagaaacagaagttacttaaatggagttcagatttaaaacaagaacgagaacaactagagcaaaaggtgaaacagctcagcaattccataagtaaatgcatggaaatgaagaacaccatcctggcccggcagaaggagatgcacagctccctggagaaggtaaaacagctgattcgcctcatccacggcatcgacctctccaaacctgtagactctgaggccactgtgggggccatctccaatggcccggactgcaccccccctgccaatgccgccacctccacgccggccccttccccctcctcccagagctgcacagcgaactgtaaccagggggaagagactaaataacagagcccctctaggagaagccacgggatcccggcggcaaggagaacagaacactgaagactctagaaaagcaaagccggatttctggaaagtgcagaattcttttggttctttggttccagagagagagaagatgcttgtgccaggtggcaccagagtttgccaattgatccttcttattctgtgtgtacatgcaaagattggaccatgttacatgaaatagtgccagctggaggttctttgccagcaccatgccaagtgaaataatatatttactctctctattatacaccagtgtgtgcctgcagcagcctccacagccacgatgggtttgtttctgttttcttgggtggggagcagggacgggcggagggaggagagcaggtttcagatccttacttgccgagccgtttgtttaggtagagaagacaagtccaaagagtgtgtgggctttcctgtttctaaactttcgctactataaaaccaaaaaaaggaattgagatttcaccaaccccagtgcccagaagagggaaggggagtggctggagggagcagggggtgggacagtgtatcaaataagcagtatttaatcacctctggcgggggcctcgtgcaaggggagactgacaccaagaacagccagtaggttcttctcccctgcactctgctccctgcgcggtaaccccaccactcctgaagcctgcccagtctccttccttccctgcttggtgagtcgcgcatctccgtggttatcccgctgtctcctctccaagaacaagcagagcccgggccactggcccttgcccaaggcagggaagaaggatgtgtgtgtccaggaaggaaaaaaaggtggatcagtgattttacttgaaaacaagctccatcccttttctatatttataagaagagaagatcttgagtgaagcagcacgcgacccaggtgtgtgtgaattgaatggagacgtttcttttctctttctttaatttttgtttttgttctttttttctttaaggaaagttttattttactgttcattttactttcttggtaacaaaaactaaaataaggaatagaaaagctgtttttcaggctgacagtccaattaagggtagccaagaccttgcatggtagagtaggaatcatagtgtcagtgaggtcccgtgagtctttgtgagtccttgtgtcatcgttcgggcactgtttttttatgcaagggcaaaaatctttgtatctggggaaaaaaaacttttttttaaattaaaaaggaaaataaaagatattgaggtcttcctagtgttacttaaattaagatcaaggtaagaaacattgtaaaaaaaaattacaaaagtgctatttgtttcctaaaaacagtgatttctattaaaaaggtgtcagaactggagaaaatgccgtgtagttataattttttagcacagaccctgctgatcacgatgacattttgccgtgtgtgtgtctctagactggtgggccagtctccttgaaggacagaggcggagctccccacccttctctctcctcagaaaagaccgtgctctcttcttggtgcagggatcttgtctcctgttgtgaagcccaaatggaagcgtggatggtatcagggccctacccgtggtcttctcagattctgctagagcaaaaggctggtgcctaaataagatcccttcctttggtgctgcttttggtctttcagccaccagcattatgagtgcctgggggacacctccgagggaactggccagcggagctctgtggtgcgcacgcaccctggccgtgacaggagggtgcgggagtacaggctggctgcatcagcccttggtgcttagaacagaggaggagtgacatgttttgagggtacgtctctgagacagagccccagcgtggccttcgctctgtcttgcctttggggagaggtctgaagctcccactcctttctctgcctgttggctccaggcaccagaaatttactccactccacccacccacaagcctcctgggtgaccctgggctagaattgctgcgcttgcctcggcttggccggttgtggcctctccttgagaaaaccagggttgtgaaagactcagaccattctctcatcttgccttgtcagaagtaaattgtgtcagatttgtgctctcgctggagacctttgccccttgcgtgcccctggccgatgggagggcggtggaggctctgtaccctggccctgctggagcatctcccccaagcccactccaggccctgggaatggccagagtctaggagaggtagaaacgatcctatcagcttctctcccacccaattaggcccagagagacaaagacagatctgaaagcaaatgcaacagagaagagacacttcttagagtaaaatgtgtctcatctctatcagccatcgcctttcatcttcccaggggcctcagaagaaggaattaagttaggctgaacaggcctcagagttaggccctggctgcttgattggctgagggggaaagagttcccttttctcattcagaaaccaaggtgctgtgtctagtcagggagccttggagatgcctggactagttggaggaatcgttggcagaggatcagagaccagcagcaggctgtctgccctgtctagagctcttcccctcaacttgtctgggcccatctgggggttgccacacaacacctaacttaccttttcctgaaagaagttgggaaaccatcatcactagaggcctttgctcagagaggagctgccttaggagtcttgggtcggaggacggggctaggaattgaccagggctttgcctgccgccctcagcagtgtcgggtacattctgacctcgcctgcagctgggctgtggattcttcctgacattcagatgtgagctgttttgggagtcagctagtatggagtacgagatgcaacccagcccccaaacctacattctgcactcaaattccaaaacactgctttactgtaaagaagaggcccctggcacccaatctccctgtccttcactgtcccctcagacctgggcggggaggggggggggcctgtgaccacctgagacatacgctcgtgacactgccccaccccagccacctccacttgcttcctcctccttccctccgctgctctttccccacggcccagaatttagctgctctgacagccacttttgagaccagctggctttgtagtcacttcagagagctggagcggctgcccactgggccctgactgggagtcccctgccagctcctgatcaggcgctgcgccctggtggcagtgatgactgggagtcccctgccagctcctgtccaggcgctgcatcctggtaacagtgaggccatgttgctgtcatctccacctctgcattcttgctgcctgtgggtcctttttctttcatggagcctgctgggtcttgtctcacctgtgctgagctcctctggggttttgatttcttccttccttatcaggccctttggggtaagcctgctggttgtacctgacatagggaggcagttaggggcagtccctggtggggccgccctggcagcctccagctggcaccatcgtgtgcctggtttccctgcaacacctgcctctctgtccctgctgctgcttggctcaggcccaacaggcagcgtgcatggaggtggttacacacagctgtttccgtgagggtgaccgtgtctgcagcacgcttccgtctccgcatgcacggctgcctctccagccacctctgatacttctctcttggggccatcagagcctcccttgggctgtcacctcccagctcacacacactcttcagtggtttcctctcttcattctcttatagggcgtggtccttcttatttatctaaagggctgaatttaggagactttttacccaggggcaaaaggctcttagggtaatgagatggatggtggcccaggtgcattttccagggcctgggttctccagatcccgtggcttctgttgagtggaggcaactttgctctgtgtgaacctcgcccctgtccctctgccgggcacccctggcaggaagcaggactcccatcctcaccctgacttagactgtcctctgagtcagctcctctccaagacaggagtgggcagccctgggcagtcttctggccccttgctaaagtgaggggcaggaagctggggctgccctccagaaagccggggtaggaactctgaaaaatacctcctctaaacggaagcagggctctccagttccacttggcgccccctcccacaaggcccttcctccctgaggaccccacccccctaccccttccccagcagcctttggaccctcacctctctccggtgtccgtgggtcctcagcccagggtgagctgcagtcaggcgggatgggacgggcaggccagaggtcagccagctcctagcagagaagagccagccagaccccaaccctgtctcttgtccatgccctttgtgatttcagtcttggtagacttgtatttggagttttgtgcttcaaagtttttgtttttgtttgtttggtttttgttttgagggggtggggggggatacagagcagctgatcaatttgtatttatttattttaacattttactaaataaagccaaataaagcctctcaaaaaaaaaaaaaaaaa

SEQ ID NO: 29 - DNA, 14135 bp, Homo sapiens
  source 1..14135: mol_type = other DNA
gcactgcagcgccagcgtccgagcgggcggccgagctcccggagcggcctggccccgagccccgagcgggcgtcgctcagcagcaggtcgcggccgcagccccatccagccccgcgcccgccatgccgtccgcgggccccgcctgagctgcggcctccgcgcgcgggcgggcctggggacggcggggccatgcgcgcgctgccctaacgatgccgcccgccgcgcccgcccgcctggcgctggccctgggcctgggcctgtggctcggggcgctggcggggggccccgggcgcggctgcgggccctgcgagcccccctgcctctgcggcccagcgcccggcgccgcctgccgcgtcaactgctcgggccgcgggctgcggacgctcggtcccgcgctgcgcatccccgcggacgccacagcgctagacgtctcccacaacctgctccgggcgctggacgttgggctcctggcgaacctctcggcgctggcagagctggatataagcaacaacaagatttctacgttagaagaaggaatatttgctaatttatttaatttaagtgaaataaacctgagtgggaacccgtttgagtgtgactgtggcctggcgtggctgccgcgatgggcggaggagcagcaggtgcgggtggtgcagcccgaggcagccacgtgtgctgggcctggctccctggctggccagcctctgcttggcatccccttgctggacagtggctgtggtgaggagtatgtcgcctgcctccctgacaacagctcaggcaccgtggcagcagtgtccttttcagctgcccacgaaggcctgcttcagccagaggcctgcagcgccttctgcttctccaccggccagggcctcgcagccctctcggagcagggctggtgcctgtgtggggcggcccagccctccagtgcctcctttgcctgcctgtccctctgctccggccccccgccacctcctgcccccacctgtaggggccccaccctcctccagcacgtcttccctgcctccccaggggccaccctggtggggccccacggacctctggcctctggccagctagcagccttccacatcgctgccccgctccctgtcactgccacacgctgggacttcggagacggctccgccgaggtggatgccgctgggccggctgcctcgcatcgctatgtgctgcctgggcgctatcacgtgacggccgtgctggccctgggggccggctcagccctgctggggacagacgtgcaggtggaagcggcacctgccgccctggagctcgtgtgcccgtcctcggtgcagagtgacgagagccttgacctcagcatccagaaccgcggtggttcaggcctggaggccgcctacagcatcgtggccctgggcgaggagccggcccgagcggtgcacccgctctgcccctcggacacggagatcttccctggcaacgggcactgctaccgcctggtggtggagaaggcggcctggctgcaggcgcaggagcagtgtcaggcctgggccggggccgccctggcaatggtggacagtcccgccgtgcagcgcttcctggtctcccgggtcaccaggagcctagacgtgtggatcggcttctcgactgtgcagggggtggaggtgggcccagcgccgcagggcgaggccttcagcctggagagctgccagaactggctgcccggggagccacacccagccacagccgagcactgcgtccggctcgggcccaccgggtggtgtaacaccgacctgtgctcagcgccgcacagctacgtctgcgagctgcagcccggaggcccagtgcaggatgccgagaacctcctcgtgggagcgcccagtggggacctgcagggacccctgacgcctctggcacagcaggacggcctctcagccccgcacgagcccgtggaggtcatggtattcccgggcctgcgtctgagccgtgaagccttcctcaccacggccgaatttgggacccaggagctccggcggcccgcccagctgcggctgcaggtgtaccggctcctcagcacagcagggaccccggagaacggcagcgagcctgagagcaggtccccggacaacaggacccagctggcccccgcgtgcatgccagggggacgctggtgccctggagccaacatctgcttgccgctggacgcctcctgccacccccaggcctgcgccaatggctgcacgtcagggccagggctacccggggccccctatgcgctatggagagagttcctcttctccgttcccgcggggccccccgcgcagtactcggtcaccctccacggccaggatgtcctcatgctccctggtgacctcgttggcttgcagcacgacgctggccctggcgccctcctgcactgctcgccggctcccggccaccctggtccccaggccccgtacctctccgccaacgcctcgtcatggctgccccacttgccagcccagctggagggcacttgggcctgccctgcctgtgccctgcggctgcttgcagccacggaacagctcaccgtgctgctgggcttgaggcccaaccctggactgcggctgcctgggcgctatgaggtccgggcagaggtgggcaatggcgtgtccaggcacaacctctcctgcagctttgacgtggtctccccagtggctgggctgcgggtcatctaccctgccccccgcgacggccgcctctacgtgcccaccaacggctcagccttggtgctccaggtggactctggtgccaacgccacggccacggctcgctggcctgggggcagtgtcagcgcccgctttgagaatgtctgccctgccctggtggccaccttcgtgcccggctgcccctgggagaccaacgataccctgttctcagtggtagcactgccgtggctcagtgagggggagcacgtggtggacgtggtggtggaaaacagcgccagccgggccaacctcagcctgcgggtgacggcggaggagcccatctgtggcctccgcgccacgcccagccccgaggcccgtgtactgcagggagtcctagtgaggtacagccccgtggtggaggccggctcggacatggtcttccggtggaccatcaacgacaagcagtccctgaccttccagaacgtggtcttcaatgtcatttatcagagcgcggcggtcttcaagctctcactgacggcctccaaccacgtgagcaacgtcaccgtgaactacaacgtaaccgtggagcggatgaacaggatgcagggtctgcaggtctccacagtgccggccgtgctgtcccccaatgccacgctagcactgacggcgggcgtgctggtggactcggccgtggaggtggccttcctgtggacctttggggatggggagcaggccctccaccagttccagcctccgtacaacgagtccttcccggttccagacccctcggtggcccaggtgctggtggagcacaatgtcatgcacacctacgctgccccaggtgagtacctcctgaccgtgctggcatctaatgccttcgagaacctgacgcagcaggtgcctgtgagcgtgcgcgcctccctgccctccgtggctgtgggtgtgagtgacggcgtcctggtggccggccggcccgtcaccttctacccgcacccgctgccctcgcctgggggtgttctttacacgtgggacttcggggacggctcccctgtcctgacccagagccagccggctgccaaccacacctatgcctcgaggggcacctaccacgtgcgcctggaggtcaacaacacggtgagcggtgcggcggcccaggcggatgtgcgcgtctttgaggagctccgcggactcagcgtggacatgagcctggccgtggagcagggcgcccccgtggtggtcagcgccgcggtgcagacgggcgacaacatcacgtggaccttcgacatgggggacggcaccgtgctgtcgggcccggaggcaacagtggagcatgtgtacctgcgggcacagaactgcacagtgaccgtgggtgcggccagccccgccggccacctggcccggagcctgcacgtgctggtcttcgtcctggaggtgctgcgcgttgaacccgccgcctgcatccccacgcagcctgacgcgcggctcacggcctacgtcaccgggaacccggcccactacctcttcgactggaccttcggggatggctcctccaacacgaccgtgcgggggtgcccgacggtgacacacaacttcacgcggagcggcacgttccccctggcgctggtgctgtccagccgcgtgaacagggcgcattacttcaccagcatctgcgtggagccagaggtgggcaacgtcaccctgcagccagagaggcagtttgtgcagctcggggacgaggcctggctggtggcatgtgcctggcccccgttcccctaccgctacacctgggactttggcaccgaggaagccgcccccacccgtgccaggggccctgaggtgacgttcatctaccgagacccaggctcctatcttgtgacagtcaccgcgtccaacaacatctctgctgccaatgactcagccctggtggaggtgcaggagcccgtgctggtcaccagcatcaaggtcaatggctcccttgggctggagctgcagcagccgtacctgttctctgctgtgggccgtgggcgccccgccagctacctgtgggatctgggggacggtgggtggctcgagggtccggaggtcacccacgcttacaacagcacaggtgacttcaccgttagggtggccggctggaatgaggtgagccgcagcgaggcctggctcaatgtgacggtgaagcggcgcgtgcgggggctcgtcgtcaatgcaagccgcacggtggtgcccctgaatgggagcgtgagcttcagcacgtcgctggaggccggcagtgatgtgcgctattcctgggtgctctgtgaccgctgcacgcccatccctgggggtcctaccatctcttacaccttccgctccgtgggcaccttcaatatcatcgtcacggctgagaacgaggtgggctccgcccaggacagcatcttcgtctatgtcctgcagctcatagaggggctgcaggtggtgggcggtggccgctacttccccaccaaccacacggtacagctgcaggccgtggttagggatggcaccaacgtctcctacagctggactgcctggagggacaggggcccggccctggccggcagcggcaaaggcttctcgctcaccgtgctcgaggccggcacctaccatgtgcagctgcgggccaccaacatgctgggcagcgcctgggccgactgcaccatggacttcgtggagcctgtggggtggctgatggtggccgcctccccgaacccagctgccgtcaacacaagcgtcaccctcagtgccgagctggctggtggcagtggtgtcgtatacacttggtccttggaggaggggctgagctgggagacctccgagccatttaccacccatagcttccccacacccggcctgcacttggtcaccatgacggcagggaacccgctgggctcagccaacgccaccgtggaagtggatgtgcaggtgcctgtgagtggcctcagcatcagggccagcgagcccggaggcagcttcgtggcggccgggtcctctgtgcccttttgggggcagctggccacgggcaccaatgtgagctggtgctgggctgtgcccggcggcagcagcaagcgtggccctcatgtcaccatggtcttcccggatgctggcaccttctccatccggctcaatgcctccaacgcagtcagctgggtctcagccacgtacaacctcacggcggaggagcccatcgtgggcctggtgctgtgggccagcagcaaggtggtggcgcccgggcagctggtccattttcagatcctgctggctgccggctcagctgtcaccttccgcctgcaggtcggcggggccaaccccgaggtgctccccgggccccgtttctcccacagcttcccccgcgtcggagaccacgtggtgagcgtgcggggcaaaaaccacgtgagctgggcccaggcgcaggtgcgcatcgtggtgctggaggccgtgagtgggctgcaggtgcccaactgctgcgagcctggcatcgccacgggcactgagaggaacttcacagcccgcgtgcagcgcggctctcgggtcgcctacgcctggtacttctcgctgcagaaggtccagggcgactcgctggtcatcctgtcgggccgcgacgtcacctacacgcccgtggccgcggggctgttggagatccaggtgcgcgccttcaacgccctgggcagtgagaaccgcacgctggtgctggaggttcaggacgccgtccagtatgtggccctgcagagcggcccctgcttcaccaaccgctcggcgcagtttgaggccgccaccagccccagcccccggcgtgtggcctaccactgggactttggggatgggtcgccagggcaggacacagatgagcccagggccgagcactcctacctgaggcctggggactaccgcgtgcaggtgaacgcctccaacctggtgagcttcttcgtggcgcaggccacggtgaccgtccaggtgctggcctgccgggagccggaggtggacgtggtcctgcccctgcaggtgctgatgcggcgatcacagcgcaactacttggaggcccacgttgacctgcgcgactgcgtcacctaccagactgagtaccgctgggaggtgtatcgcaccgccagctgccagcggccggggcgcccagcgcgtgtggccctgcccggcgtggacgtgagccggcctcggctggtgctgccgcggctggcgctgcctgtggggcactactgctttgtgtttgtcgtgtcatttggggacacgccactgacacagagcatccaggccaatgtgacggtggcccccgagcgcctggtgcccatcattgagggtggctcataccgcgtgtggtcagacacacgggacctggtgctggatgggagcgagtcctacgaccccaacctggaggacggcgaccagacgccgctcagtttccactgggcctgtgtggcttcgacacagagggaggctggcgggtgtgcgctgaactttgggccccgcgggagcagcacggtcaccattccacgggagcggctggcggctggcgtggagtacaccttcagcctgaccgtgtggaaggccggccgcaaggaggaggccaccaaccagacggtgctgatccggagtggccgggtgcccattgtgtccttggagtgtgtgtcctgcaaggcacaggccgtgtacgaagtgagccgcagctcctacgtgtacttggagggccgctgcctcaattgcagcagcggctccaagcgagggcggtgggctgcacgtacgttcagcaacaagacgctggtgctggatgagaccaccacatccacgggcagtgcaggcatgcgactggtgctgcggcggggcgtgctgcgggacggcgagggatacaccttcacgctcacggtgctgggccgctctggcgaggaggagggctgcgcctccatccgcctgtcccccaaccgcccgccgctggggggctcttgccgcctcttcccactgggcgctgtgcacgccctcaccaccaaggtgcacttcgaatgcacgggctggcatgacgcggaggatgctggcgccccgctggtgtacgccctgctgctgcggcgctgtcgccagggccactgcgaggagttctgtgtctacaagggcagcctctccagctacggagccgtgctgcccccgggtttcaggccacacttcgaggtgggcctggccgtggtggtgcaggaccagctgggagccgctgtggtcgccctcaacaggtctttggccatcaccctcccagagcccaacggcagcgcaacggggctcacagtctggctgcacgggctcaccgctagtgtgctcccagggctgctgcggcaggccgatccccagcacgtcatcgagtactcgttggccctggtcaccgtgctgaacgagtacgagcgggccctggacgtggcggcagagcccaagcacgagcggcagcaccgagcccagatacgcaagaacatcacggagactctggtgtccctgagggtccacactgtggatgacatccagcagatcgctgctgcgctggcccagtgcatggggcccagcagggagctcgtatgccgctcgtgcctgaagcagacgctgcacaagctggaggccatgatgctcatcctgcaggcagagaccaccgcgggcaccgtgacgcccaccgccatcggagacagcatcctcaacatcacaggagacctcatccacctggccagctcggacgtgcgggcaccacagccctcagagctgggagccgagtcaccatctcggatggtggcgtcccaggcctacaacctgacctctgccctcatgcgcatcctcatgcgctcccgcgtgctcaacgaggagcccctgacgctggcgggcgaggagatcgtggcccagggcaagcgctcggacccgcggagcctgctgtgctatggcggcgccccagggcctggctgccacttctccatccccgaggctttcagcggggccctggccaacctcagtgacgtggtgcagctcatctttctggtggactccaatccctttccctttggctatatcagcaactacaccgtctccaccaaggtggcctcgatggcattccagacacaggccggcgcccagatccccatcgagcggctggcctcagagcgcgccatcaccgtgaaggtgcccaacaactcggactgggctgcccggggccaccgcagctccgccaactccgccaactccgttgtggtccagccccaggcctccgtcggtgctgtggtcaccctggacagcagcaaccctgcggccgggctgcatctgcagctcaactatacgctgctggacggccactacctgtctgaggaacctgagccctacctggcagtctacctacactcggagccccggcccaatgagcacaactgctcggctagcaggaggatccgcccagagtcactccagggtgctgaccaccggccctacaccttcttcatttccccggggagcagagacccagcggggagttaccatctgaacctctccagccacttccgctggtcggcgctgcaggtgtccgtgggcctgtacacgtccctgtgccagtacttcagcgaggaggacatggtgtggcggacagaggggctgctgcccctggaggagacctcgccccgccaggccgtctgcctcacccgccacctcaccgccttcggcgccagcctcttcgtgcccccaagccatgtccgctttgtgtttcctgagccgacagcggatgtaaactacatcgtcatgctgacatgtgctgtgtgcctggtgacctacatggtcatggccgccatcctgcacaagctggaccagttggatgccagccggggccgcgccatccctttctgtgggcagcggggccgcttcaagtacgagatcctcgtcaagacaggctggggccggggctcaggtaccacggcccacgtgggcatcatgctgtatggggtggacagccggagcggccaccggcacctggacggcgacagagccttccaccgcaacagcctggacatcttccggatcgccaccccgcacagcctgggtagcgtgtggaagatccgagtgtggcacgacaacaaagggctcagccctgcctggttcctgcagcacgtcatcgtcagggacctgcagacggcacgcagcgccttcttcctggtcaatgactggctttcggtggagacggaggccaacgggggcctggtggagaaggaggtgctggccgcgagcgacgcagcccttttgcgcttccggcgcctgctggtggctgagctgcagcgtggcttctttgacaagcacatctggctctccatatgggaccggccgcctcgtagccgtttcactcgcatccagagggccacctgctgcgttctcctcatctgcctcttcctgggcgccaacgccgtgtggtacggggctgttggcgactctgcctacagcacggggcatgtgtccaggctgagcccgctgagcgtcgacacagtcgctgttggcctggtgtccagcgtggttgtctatcccgtctacctggccatcctttttctcttccggatgtcccggagcaaggtggctgggagcccgagccccacacctgccgggcagcaggtgctggacatcgacagctgcctggactcgtccgtgctggacagctccttcctcacgttctcaggcctccacgctgaggcctttgttggacagatgaagagtgacttgtttctggatgattctaagagtctggtgtgctggccctccggcgagggaacgctcagttggccggacctgctcagtgacccgtccattgtgggtagcaatctgcggcagctggcacggggccaggcgggccatgggctgggcccagaggaggacggcttctccctggccagcccctactcgcctgccaaatccttctcagcatcagatgaagacctgatccagcaggtccttgccgagggggtcagcagcccagcccctacccaagacacccacatggaaacggacctgctcagcagcctgtccagcactcctggggagaagacagagacgctggcgctgcagaggctgggggagctggggccacccagcccaggcctgaactgggaacagccccaggcagcgaggctgtccaggacaggactggtggagggtctgcggaagcgcctgctgccggcctggtgtgcctccctggcccacgggctcagcctgctcctggtggctgtggctgtggctgtctcagggtgggtgggtgcgagcttccccccgggcgtgagtgttgcgtggctcctgtccagcagcgccagcttcctggcctcattcctcggctgggagccactgaaggtcttgctggaagccctgtacttctcactggtggccaagcggctgcacccggatgaagatgacaccctggtagagagcccggctgtgacgcctgtgagcgcacgtgtgccccgcgtacggccaccccacggctttgcactcttcctggccaaggaagaagcccgcaaggtcaagaggctacatggcatgctgcggagcctcctggtgtacatgctttttctgctggtgaccctgctggccagctatggggatgcctcatgccatgggcacgcctaccgtctgcaaagcgccatcaagcaggagctgcacagccgggccttcctggccatcacgcggtctgaggagctctggccatggatggcccacgtgctgctgccctacgtccacgggaaccagtccagcccagagctggggcccccacggctgcggcaggtgcggctgcaggaagcactctacccagaccctcccggccccagggtccacacgtgctcggccgcaggaggcttcagcaccagcgattacgacgttggctgggagagtcctcacaatggctcggggacgtgggcctattcagcgccggatctgctgggggcatggtcctggggctcctgtgccgtgtatgacagcgggggctacgtgcaggagctgggcctgagcctggaggagagccgcgaccggctgcgcttcctgcagctgcacaactggctggacaacaggagccgcgctgtgttcctggagctcacgcgctacagcccggccgtggggctgcacgccgccgtcacgctgcgcctcgagttcccggcggccggccgcgccctggccgccctcagcgtccgcccctttgcgctgcgccgcctcagcgcgggcctctcgctgcctctgctcacctcggtgtgcctgctgctgttcgccgtgcacttcgccgtggccgaggcccgtacttggcacagggaagggcgctggcgcgtgctgcggctcggagcctgggcgcggtggctgctggtggcgctgacggcggccacggcactggtacgcctcgcccagctgggtgccgctgaccgccagtggacccgtttcgtgcgcggccgcccgcgccgcttcactagcttcgaccaggtggcgcagctgagctccgcagcccgtggcctggcggcctcgctgctcttcctgcttttggtcaaggctgcccagcagctacgcttcgtgcgccagtggtccgtctttggcaagacattatgccgagctctgccagagctcctgggggtcaccttgggcctggtggtgctcggggtagcctacgcccagctggccatcctgctcgtgtcttcctgtgtggactccctctggagcgtggcccaggccctgttggtgctgtgccctgggactgggctctctaccctgtgtcctgccgagtcctggcacctgtcacccctgctgtgtgtggggctctgggcactgcggctgtggggcgccctacggctgggggctgttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttagtcctccttcctggcgggggtgggccgtggagtcggagtggacaccgctcagtattactttctgccgctgtcaaggccgagggccaggcagaatggctgcacgtaggttccccagagagcaggcaggggcatctgtctgtctgtgggcttcagcactttaaagaggctgtgtggccaaccaggacccagggtcccctccccagctcccttgggaaggacacagcagtattggacggtttctagcctctgagatgctaatttatttccccgagtcctcaggtacagcgggctgtgcccggccccaccccctgggcagatgtcccccactgctaaggctgctggcttcagggagggttagcctgcaccgccgccaccctgcccctaagttattacctctccagttcctaccgtactccctgcaccgtctcactgtgtgtctcgtgtcagtaatttatatggtgttaaaatgtgtatatttttgtatgtcactattttcactagggctgaggggcctgcgcccagagctggcctcccccaacacctgctgcgcttggtaggtgtggtggcgttatggcagcccggctgctgcttggatgcgagcttggccttgggccggtgctgggggcacagctgtctgccaggcactctcatcaccccagaggccttgtcatcctcccttgccccaggccaggtagcaagagagcagcgcccaggcctgctggcatcaggtctgggcaagtagcaggactaggcatgtcagaggaccccagggtggttagaggaaaagactcctcctgggggctggctcccagggtggaggaaggtgactgtgtgtgtgtgtgtgtgcgcgcgcgcacgcgcgagtgtgctgtatggcccaggcagcctcaaggccctcggagctggctgtgcctgcttctgtgtaccacttctgtgggcatggccgcttctagagcctcgacacccccccaacccccgcaccaagcagacaaagtcaataaaagagctgtctgactgc

SEQ ID NO: 30 - DNA, 2207 bp, Homo sapiens
  source 1..2207: mol_type = other DNA
gcatcctctcaccgccggaagctgaactgactcgtccgcggccgctctaccccaacaggccgccaccagcgagagtgcggccataaccatcacgtgaccgcccaccgacaccagcgagagtgcagtcgtaaccgtcacgtgaccgcccaccgtcggcccggcgctcccctccgcccgaagctagcaagcggcgcggccaatgagaaaggcgcatgcctggcccccgccggcctgcagtctagccgtagtgcgcctgcgcgcggctaggaggggccgtcaggcggggatacagcctggaaggtaatgcatgtccatggtacacaaattcacaagtttggagaccctgacacacccaccttctcacctgggctctgcgtatcccccagccttgagggaagatgaagcctaaactgatgtaccaggagctgaaggtgcctgcagaggagcccgccaatgagctgcccatgaatgagattgaggcgtggaaggctgcggaaaagaaagcccgctgggtcctgctggtcctcattctggcggttgtgggcttcggagccctgatgactcagctgtttctatgggaatacggcgacttgcatctctttgggcccaaccagcgcccagccccctgctatgacccttgcgaagcagtgctggtggaaagcattcctgagggcctggacttccccaatgcctccacggggaacccttccaccagccaggcctggctgggcctgctcgccggtgcgcacagcagcctggacatcgcctccttctactggaccctcaccaacaatgacacccacacgcaggagccctctgcccagcagggtgaggaggtcctccggcagctgcagaccctggcaccaaagggcgtgaacgtccgcatcgctgtgagcaagcccagcgggccccagccacaggcggacctgcaggctctgctgcagagcggtgcccaggtccgcatggtggacatgcagaagctgacccatggcgtcctgcataccaagttctgggtggtggaccagacccacttctacctgggcagtgccaacatggactggcgttcactgacccaggtcaaggagctgggcgtggtcatgtacaactgcagctgcctggctcgagacctgaccaagatctttgaggcctactggttcctgggccaggcaggcagctccatcccatcaacttggccccggttctatgacacccgctacaaccaagagacaccaatggagatctgcctcaatggaacccctgctctggcctacctggcgagtgcgcccccacccctgtgtccaagtggccgcactccagacctgaaggctctactcaacgtggtggacaatgcccggagtttcatctacgtcgctgtcatgaactacctgcccactctggagttctcccaccctcacaggttctggcctgccattgacgatgggctgcggcgggccacctacgagcgtggcgtcaaggtgcgcctgctcatcagctgctggggacactcggagccatccatgcgggccttcctgctctctctggctgccctgcgtgacaaccatacccactctgacatccaggtgaaactctttgtggtccccgcggatgaggcccaggctcgaatcccatatgcccgtgtcaaccacaacaagtacatggtgactgaacgcgccacctacatcggaacctccaactggtctggcaactacttcacggagacggcgggcacctcgctgctggtgacgcagaatgggaggggcggcctgcggagccagctggaggccattttcctgagggactgggactccccttacagccatgaccttgacacctcagctgacagcgtgggcaacgcctgccgcctgctctgaggcccgatccagtgggcaggccaaggcctgctgggcccccgcggacccaggtgctctgggtcacggtccctgtccccgcgcccccgcttctgtctgccccattgtggctcctcaggctctctcccctgctctcccacctctacctccacccccaccggcctgacgctgtggccccgggacccagcagagctgggggagggatcagcccccaaagaaatgggggtgcatgctgggcctggccccctggcccacccccactttccagggcaaaaagggcccagggttataataagtaaataacttgtctgtacagcctgaaaaaaaaaaaaaaaaaaa

SEQ ID NO: 31 - DNA, 4846 bp, Homo sapiens
  source 1..4846: mol_type = other DNA
gagcggtgctcaggggagggctggaggggagggaaggagagagagaggggagggcggcaccgcccctagccccgcgctccggaagtgaagcggccagaccaccagctaatggatgcggagcggagggcccgctgaccgctctccgcgcctggagcagcttggcttggctggagctaagagccagacacaccactgtgtggaggtgggtgatgtcttcctgtgctaaaaggtgaataaataagctcctcacctctcgcggaacactcgggaacacatcaacaggggtccaagccgccctgctgggaggcttctcttcaagagttctgggtcccagagtggaaggcattttcccatcaactggagagagacgaaacatcagagaccaggaggctgtggagaaagcagctgtcccaggtgcctcaactatcagagaagggtcagcgtcacgtggctgccagcatctttgagaaaatcactggcaatcggacttcagagctgcgggcacaggtgtggttagaactgagatacgacctgcccacctgggtcaggcctaaagacaagaagtcctgagttcttgccactgagtaggccagggtcatttgtccagaaaactttgtgactgtctttgagtgacctagtctgggacccattcattggtgggttctaaggttagaagctcatccaggatattttcaatattaagtcagtgcatagctgcaccactaacaaattggtgcctgtagagtcagagtgggtcaattcttaggacaatggcgctggcactgttagaggactggtgcaggataatgagtgtggatgagcagaagtcactgatggttacggggataccggcggactttgaggaggctgagattcaggaggtccttcaggagactttaaagtctctgggcaggtatagactgcttggcaagatattccggaagcaggagaatgccaatgctgtcttactagagcttctggaagatactgatgtctcggccattcccagtgaggtccagggaaaggggggtgtctggaaggtgatctttaagacccctaatcaggacactgagtttcttgaaagattgaacctgtttctagaaaaagaggggcagacggtctcgggtatgtttcgagccctggggcaggagggcgtgtctccagccacagtgccctgcatctcaccagaattactggcccatttgttgggacaggcaatggcacatgcgcctcagcccctgctacccatgagataccggaaactgcgagtattctcagggagtgctgtcccagccccagaggaagagtcctttgaggtctggttggaacaggccacggagatagtcaaagagtggccagtaacagaggcagaaaagaaaaggtggctggcggaaagcctgcggggccctgccctggacctcatgcacatagtgcaggcagacaacccgtccatcagtgtagaagagtgtttggaggcctttaagcaagtgtttgggagcctagagagccgcaggacagcccaggtgaggtatctgaagacctatcaggaggaaggagagaaggtctcagcctatgtgttacggctagaaaccctgctccggagagcggtggagaaacgcgccatccctcggcgtattgcggaccaggtccgcctggagcaggtcatggctggggccactcttaaccagatgctgtggtgccggcttagggagctgaaggatcagggcccgccccccagcttccttgagctaatgaaggtaatacgggaagaagaggaggaagaggcctcctttgagaatgagagtatcgaagagccagaggaacgagatggctatggccgctggaatcatgagggagacgactgaaaaccacctgggggcaggacccacagccagtgggctaagacctttaaaaaatttttttctttaatgtatgggactgaaatcaaaccatgaaagccaattattgaccttccttccttccttccttccctcccttcctccttctctccttctctcctcctctctcctctcctctcctctctttccttccttccttccttttttctttttctctttcttctttatttcttgggtctcactctcatcacccaggctagagtgcagtggcacaaaaatctcggctcactgcagccttgacttcccaggctcaggctcaggtgatcctcacaccttagcctcccaagtacctgggactacaggcacgcaccaccatgcctagctattcttttgtatttttggtagagacagggttttgctgtgttgctcaggctggtctggaacccctaggctcaaatgatgtgcccaactcggcctcccaaagtgctgggattacaggcatgaaccgccatgcctggcccttgatttttctttttaagaaaaaaatatctaggagtttcttagaccctatgtagattattaatgaacaaaagattaaactccaaatattaaatagtaagcctgaaggaatctgaaacacttgtacttccaattttctttaaataatcccaaatagaccagaattggcccataccatagaagaaagaattggcagtcaaaaaaaaaaataccttttgtaatgtttgaaaaataaagctgtttgacttgtcaggtgttttcctttctcaaatcagcaaattctctctgagtgcctggctttgtgagacactgtacaaggagttacaagactacagctataacctgcagttgagcagttataaacctacaaaatgggccctgccctcagagaggttccagtctagatgaggagctgatctagacaggtaaaaggctaactaaccctttgtgtaaataagttcatcaccccagtaaaagtgtcatcacccagtgaataggaccacctctgcctgcagatttttgttgttgttgttgtcattgttgttgttgttttaacctgggaagtgttcttcctgcctttctgctaggtgtcagatagatggtcccagagctaggtgctgtgtcaggccctgaagacacagatgactcaacctaagctttactttccagaggtccacagcctgagaggtgtccccaaagaaagggggacatgaggggactgcatgcttgagagcagggttgtttagggcaggtttggatttagtgagcaggctggtttgcttagagaaggcttttagtggcaacaaaggatgaagaggagagaaaaggaactcacatttattgagggcctactgtgtgcaaagtgtttcatgtatatctcattgaatgtatacagccaccctgttgtggtataattttgctctttataaagagaaagaccgaagctcagatgagttaagtggtctcctcaacaccaaaatgccaagaagtgatggagcctagacagaagcccagaactttctgactcacactagtccatcctctaccatcacgatgactttcaaattgtgctctgcagttctgcagattttctagcagtgccatctccaaaatgtgttttaaactctttatttttttaattattattagtattattttgagactgagtcttgctctatcacccaggctggagtgcagtggtgcaatctcagctcactgcaacctccgcctcccaggttcaagcgatttcgtgcctcagcctcccgagtagctgggattacaggcacccaccaccacgcccagctaatttttgtatttttagtagaaatggggtttcaccatgttggccaggctggtctcgaactcctgacctcaagtgatccactcacctcggcctcccaaagtgctgggattacaggtgtgagccaccatgcctgggctaaactctttaagtctctagtaaatgcagctagattcaaatgggctgataaccaaattttaacacatcagcattcaccaccaggtttacttttattttcagattggctcattttgtgcagaccttagagcaaagtttcctttatggtatctgtgtacgtatccaaacttcttttaattgttcacagattttaaaagcggtagcaccacatggttgtgtagatcagacctgtgtatttagatcagacctgtgtatcacgtaagtgtgtgagtgcagtgcagatgagcaccatttagttatatgtgctaggcaaatctccaacacagttgatgtgtagtcttgtggtagatttgtgcatactgtaagcaaattgcttagcttctctagacatcagtttccacatctgaaaaataagaagatgagagtacacggttgttatgaacaaatgacttaatgctttttaagcacgttgcatgacatctggaacacagaaagccctcaatacattgaagctcttaggattttcacgatgttcctgtctgctcaatgcatgctttctttattgttctgacagttgtgtggtaacaagctaatatgcttccagttgacttccagtctaccctggtgttagaaaccgtttcatctcttattgtaaatttgagtgcttgttgttttttatatttgtgatgactcttccagcagttgttgacaattgttagaggtttgacttttaaataattacttattttttctgattgtggttcagtttaactgaagaatatcctgagattgtaagaaaagcattttttaaaaggtatcacttgtgatcatttatctttctaaattctatttttaatactgttccaccaaagtgatgcagtggttaccatgacaccctaatttcatgtgtttttgtatttatgaaaatagtttcattgtcatttattggcggtatacaaagtaaaatgttataaatgtgaagttataaaataaatatatgctaataaaatcctgagtttttctgtttcct

SEQ ID NO: 32 - DNA, 1014 bp, Homo sapiens
  source 1..1014: mol_type = other DNA
tgcctcctgagcgtagtccagttactttcaggctcggggagtgaaggcctcgttgagagaaggtctcattcggtgttttgggaagagagtcgtgtgggcccaggtctgtctgctatcagctatgccgctgcccgttgcgctgcagacccgcttggccaagagaggcatcctcaaacatctggagcctgaaccagaggaagagatcattgccgaggactatgacgatgatcctgtggactacgaggccaccaggttggagggcctaccaccaagctggtacaaggtgttcgacccttcctgcgggctcccttactactggaatgcagacacagaccttgtatcctggctctccccacatgaccccaactccgtggttaccaaatcggccaagaagctcagaagcagtaatgcagatgctgaagaaaagttggaccggagccatgacaagtcggacaggggccatgacaagtcggaccgcagccatgagaaactagacaggggccacgacaagtcagaccggggccacgacaagtctgacagggatcgagagcgtggctatgacaaggtagacagagagagagagcgagacagggaacgggatcgggaccgcgggtatgacaaggcagaccgggaagagggcaaagaacggcgccaccatcgccgggaggagctggctccctatcccaagagcaagaaggcagtaagccgaaaggatgaagagttagaccccatggaccctagctcatactcagacgccccccggggcacgtggtcaacaggactccccaagcggaatgaggccaagactggcgctgacaccacagcagctgggcccctcttccagcagcggccgtatccatccccaggggctgtgctccgggccaatgcagaggcctcccgaaccaagcagcaggattgaagcttcggcctccctggccctgggttaaaataaaagctttctggtgatcctgcccaccaaaaaaaaaaaaaaaaaaaaaaaaaaaaaaaaaaaaa

SEQ ID NO: 33 - DNA, 3291 bp, Homo sapiens
  source 1..3291: mol_type = other DNA
agaatcggagagccggtggcgtcgcaggtcgggaggacgagcaccgagtcgagggctcgctcgtctgggccgcccgagagtcttaatcgcgggcgcttgggccgccatcttagatggcgggagtaagaggaaaacgattgtgaggcgggaacggctttctgctgccttttttgggccccgaaaagggtcagctggccgggctttggggcgcgtgccctgaggcgcggagcgcgtttgctacgatgcgggggctgctcggggctccgtcccctgggctggggacgcgccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccaccctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctgctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcagggagcagtcttccatcatgctgaattttgtcttccaggagctgcccctatggggcggggccgcagggccagccttgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaatttaggtgtaaaaaaaaaaaaaaaaaa

SEQ ID NO: 34 - DNA, 5216 bp, Homo sapiens
  source 1..5216: mol_type = other DNA
gatgtcccaggggtattggggcggggggttgaaataactggggttcaggaggagggatggtggtagagataaaaatgtgagaagggagcagcactggcgaggagtcgggagagtactcctgattgtgacatcacattcatcccctgggcgatggagcttgtcactgggaaggaatactcagtcggagaatagccaacaagatgggttactgggagaatctcttcagtggcactgagtggaggcatcagggggttggagccttgtgaacagggaacctgccccccaacacttggaaggacctgggtttcagtgatgagacatggggtatgatgtaacccgtttccagggggatgttgacgaagatcttatctgccctatttgcagtggagtcttggaggagccagtacaggcacctcattgtgaacatgctttctgcaacgcctgcatcacccagtggttctctcagcaacagacatgtccagtggaccgtagtgttgtgacggtcgcccatctgcgcccagtacctcggatcatgcggaacatgttgtcaaagctgcagattgcctgtgacaacgctgtgttcggctgtagtgccgttgtccggcttgacaacctcatgtctcacctcagcgactgtgagcacaacccgaagcggcctgtgacctgtgaacagggctgtggcctggagatgcccaaagatgagctgcccaaccataactgcattaagcacctgcgctcagtggtacagcagcagcagacacgcatcgcagagctggagaagacgtcagctgaacacaaacaccagctggcggagcagaagcgagacatccagctgctaaaggcatacatgcgtgcaatccgcagtgtcaaccccaaccttcagaacctggaggagacaattgaatacaacgagatcctagagtgggtgaactcccttcagccagcaagagtgacccgctggggagggatgatctcgactcctgatgctgtgctccaggctgtaatcaagcgctccctggtggagagtggctgtcctgcttctattgtcaacgagctgattgaaaatgcccacgagcgtagctggccccagggtctggccacactagagactagacagatgaaccgacgctactatgagaactacgtggccaagcgcatccctggcaagcaggctgttgtcgtgatggcctgtgagaaccagcacatgggggatgacatggtgcaagagccaggccttgtcatgatatttgcgcatggcgtggaagagatataagagaactcgactggctatcaggaagagatggaaatcagaaaatcccatcactccagcagctgggacctgagtcctacccaccattcttaatactgtggcttatacctgagccacacatctccctgcccttctggcactgaagggccttggggtagtttgctcagcctttcaggtgggaaacccagatttcctccctttgccatattcccctaaaatgtctataaattatcagtctgggtgggaaagcccccacctccatccattttcctgcttagggtccctggttccagttattttcagaaagcacaaagagattcaatttccctggaggatcaggacagaggaaggaatctctaatcgtccctctcctccaaaaccagggaatcagagcagtcaggcctgttgactctaagcagcagacatcctgaagaaatggtaagggtggagccaaatctctagaaataagtagtgaggccgttaattggccatcactgatggcccttagggaaagactggacctctgtgccaagcagtatccctgttcagcccaccttaaaggtgtaggcacccactgggtctaccagtatgcaggttgggatactgaaaatttccagatgagctcttctttcctacaagttttcataattagggaatgccagggtttagggtaggggttaatctgttgggggttgatgtgtttagcaagaagctactcctagcttttgctaaaatatggttggcactgcctcttgtggcacaggccataattgttccatagacccctctctagccctgtgactgtagttagttactttgataattttctttggccattgtttgtttatatttcacaaactccacctactgcccccccccctcttttttttaagaatggcctgatcatggctatctcagccacattgttggcaatttaatttatttacttcctttttttttttttaagaaaggaaaaaagaaaaaaaaatcaaacttgaaacttttcttttgatgttcctattgtgggggttctggatagggtgggacagggatgggggtgtgttttatattttttccttttcagcacaacctttggctttaatataggaagagccaagggagtcctcggctgaacttacgatatctgccccaaacctctgtaaccccaactgaaatgaggagcttcctctcttcctgtgaaggatatgacagtccagcatcgatgcctgtgccctctggaaaaatttcctcctagcccttccagggccttatcataaaactctggatttagagtattcattttgaaggcaactcccccttccccaagtttccttggagctgtatagctgggttctaagcttcaccatgcaaatcagaaattttatctctaagtacaggctgtgccgtgtctcacccacacccccctggggacttcagttccatttcaggttacctggggtataccttgatccctagagtgactggcagagtaagagaaggggagagataataggtgtgattattttaatatggaggtgggagtgtggttggagatagaaaggctcctccccaccatgtaatggcttcctctcagaattttattccaggctagcttgctgcaggtctgggtagttggatcatggctccactgggattggggtggaaagcttgaggggagtagggttccagctctgggacattgtgctcaggaatttgaaaacgctgctatacttactctggttactacatttcttccactcccctttcccctacctgccttaaccaaggctcatactgtcctgtccttaccctcagatggagccaggaagctcagtgaaaggcttccctaccctttgcactagtgtctctgcaggttgctggttgtgttgtatgtgctgttccatggtgttgactgcactaataataaaccttttactcaactctctaaattcttcagcattactccctttcttgagaaggtttcccctctgcttttgcctttctctcaccttaattccctttcttccttactttgttacctacccttatcttagtgctaacttctctttcaggaggatgtctgggagtagtgtgcacttcacagctgctttcccatgtaccctcctgcattcttccctcctatctcctgttctgtagcagccaaagctctctagtgatctgaactgtgtgcttcccagggtctgcctttatcctaaattccatgtcttccctgagtggtcctgagtttttgggataatttctacagaagatatgtatatatctttttcctttgtcccacaagcaactttgctttagaatctagaattcctttgcaggcagagaagtctctacctcccagtgtttcctagctaagaacgtaaatgtgaggagggaaatgtacttgcagaggtttcataattatttacttataaaaatagtcttcatagccgggcgcggtggctcacgcctgtaatcccagcactttgggaggccgaggtgggtggatcacaaggtcaggagttcgagaccatcctggctaacacagtgaaaccccgtctctactaaaaatacaaaaaattagccgggcgtggtggcaggcacctgtagtcccagctacttaggaggctgaggcaggagaatggcgtgaacccgggaggcagagcttgcagtgagcagagattgggccactgcattccagcctgggcgacagagcaaggctccgtctaaaaaaaaaaaaaaaaaaaaaagtcttcataggccgggcacggtggctcacgtctgtaatcccagcactttgggaggccaaggtgggtggatcacaacgtcaggagatcgagaccatcctggctaacatggtgaaaccctgtctctactaaaaatataaataaattagccggacaggcgcctgtcctcccagctactcaggaggctgaggcaggagaatggtgtgaacctgggaggcggagcttgcagtgagctgagatcacgccactgcactccagcctgggcaacagagcaagactccgtctcaaaaaaaaaaaaaaaaaaaccagtcttcataagtatttgctgctacctttccctgtcataagaaaaaggatagccagacatggtgggacgccactatgatcccagctccttggaaggctaaggcacaagaatcgcttgaacctgggaggtggaggttgcagtgagctgagatcatgccactgcactccagcctggtgacagagcaagagcctgtctcaaaaaaaaaaaagaaaagaaaagaaaaagggatatcttttcctcctcccagaagtttgttttaaatttgagcatttatcatgcacctgatgtaaacctaatagtactcttgatactctagtggcttgaaaaaaaaaaaaaaggcatttctgtgctgagtctgcgcttctatgcacacaaggtatgtttataaaatactgataagcatgtcacagtatagagcataagaggcaatgtatgtatcctagtgacattagcagtgcttttccccccttaaactcctttaaaattacttttagaacttgctgctcattcttgtgaatgttatgaatggtgtcatattgtccttttacagaagatacgatttttagaaacaaatattcattgaatgtctgccctgtgagatactcactagagtgaacatgaggaggcttatgtagcaaaatggcacctacctgcaaagaacttagtccctaatggagatgaatatataataagggatcataaatgtgctaagtggatttactagtaatatgtgagccaaggacgataaagctcctgattctgatgggtatcaggaaaggcttttcaggaagtgttacttgttataggtcagaggtcagcaaactacaggttacaaccccactgcctgcttttgtaaaaaactttattggaatacagttatgcccacttgtttata

SEQ ID NO: 35 - DNA, 5138 bp, Homo sapiens
  source 1..5138: mol_type = other DNA
gatccgcagaggagcccacttgagagcgcctcctgtcgtctgtaaggttgccttgccatccctcggcaccccaacttcccccgcccccccatcgcctcctcctccatcctccagttcaaaatggcgacggcggcggcagcggcggcggtgatggctcctccgggctgcccgggttcgtgccccaacttcgccgtagtctgctccttcttggagcgctacgggccgctgctagacctgcctgagttgccgttccctgagctggagcgggtgctgcaggcgccgccgccggacgtcggcaacggagaagtaccaaaagaattggtggagctccatttgaagctgatgaggaaaattggcaaatctgttactgcagacagatgggaaaaatatttgatcaagatatgccaagagtttaacagtacctgggcatgggagatggagaagaagggctatcttgaaatgagtgttgaatgcaaactagcactcttaaagtacctctgtgagtgtcagtttgatgacaatctcaaattcaagaatattattaatgaggaggatgccgatactatgcgtctccagccaattggtcgagacaaagatggcctcatgtactggtaccaattggatcaagatcacaatgtcagaatgtacatagaagaacaagatgatcaagatggctcttcatggaaatgcattgtcagaaatcgaaacgagttggctgagactcttgcactcctgaaagcacaaattgatcctgtactattgaaaaactctagccaacaagacaactcttctcgggaaagtcccagcttagaggatgaggagactaaaaaagaggaagaaacacctaaacaagaggaacagaaagaaagtgaaaagatgaaaagtgaggagcagcctatggatttagaaaaccgttctacagccaatgttctagaagagactactgtgaaaaaagaaaaagaagatgaaaaggaacttgtgaaactgccagtcatagtgaagctagaaaaacctttgccagaaaatgaagaaaaaaagattatcaaagaagaaagtgattccttcaaggaaaatgtcaaacccattaaagttgaggtgaaggaatgtagagcagatcctaaagataccaaaagtagcatggagaagccagtggcacaggagcctgaaaggatcgaatttggtggcaatattaaatcttctcacgaaattactgagaaatctactgaagaaactgagaaacttaaaaatgaccagcaggccaagataccactaaaaaaacgagaaattaaactgagtgatgattttgacagtccagtcaagggacctttgtgtaaatcagttactccaacaaaagagtttttgaaagatgaaataaaacaagaggaagagacttgtaaaaggatctctacaatcactgctttgggtcatgaagggaaacagctggtaaatggagaagttagtgatgaaagggtagctccaaattttaagacagaaccaatagagacaaagttttatgagacaaaggaagagagctatagcccctctaaggacagaaatatcatcacggagggaaatggaacagagtccttaaattctgtcataacaagtatgaaaacaggtgagcttgagaaagaaacagcccctttgaggaaagatgcagatagttcaatatcagtcttagagatccatagtcaaaaagcacaaatagaggaacccgatcctccagaaatggaaacttctcttgattcttctgagatggcaaaagatctctcttcaaaaactgctttatcttccaccgagtcgtgtaccatgaaaggtgaagagaagtctcccaaaactaagaaggataagcgcccaccaatcctagaatgtcttgaaaagttagagaagtccaaaaagacttttcttgataaggacgcacaaagattgagtccaataccagaagaagttccaaagagtactctagagtcagaaaagcctggctctcctgaggcagctgaaacttctccaccatctaatatcattgaccactgtgagaaactagcctcagaaaaagaagtggtagaatgccagagtacaagtactgttggtggccagtctgtgaaaaaagtagacctagaaaccctaaaagaggattctgagttcacaaaggtagaaatggataatctggacaatgcccagacctctggcatagaggagccttctgagacaaagggttctatgcaaaaaagcaaattcaaatataagttggttcctgaagaagaaaccactgcctcagaaaatacagagataacctctgaaaggcagaaagagggcatcaaattaacaatcaggatatcaagtcggaaaaagaagcccgattctccccccaaagttctagaaccagaaaacaagcaagagaagacagaaaaggaagaggagaaaacaaatgtgggtcgtactttaagaagatctccaagaatatctagacccactgcaaaagtggctgagatcagagatcagaaagctgataaaaaaagaggggaaggagaagatgaggtggaagaagagtcaacagctttgcaaaaaactgacaaaaaggaaattttgaaaaaatcagagaaagatacaaattctaaagtaagcaaggtaaaacccaaaggcaaagttcgatggactggttctcggacacgtggcagatggaaatattccagcaatgatgaaagtgaagggtctggcagtgaaaaatcatctgcagcttcagaagaggaggaagaaaaggaaagtgaagaagccatcctagcagatgatgatgaaccatgcaaaaaatgtggccttccaaaccatcctgagctaattcttctgtgtgactcttgcgatagtggataccatactgcctgccttcgccctcctctgatgatcatcccagatggagaatggttctgcccaccttgccaacataaactgctctgtgaaaaattagaggaacagttgcaggatttggatgttgccttaaagaagaaagagcgtgccgaacgaagaaaagaacgcttggtgtatgttggtatcagtattgaaaacatcattcctccacaagagccagacttttctgaagatcaagaagaaaagaaaaaagattcaaaaaaatccaaagcaaacttgcttgaaaggaggtcaacaagaacaaggaaatgtataagctacagatttgatgagtttgatgaagcaattgatgaagctattgaagatgacatcaaagaagccgatggaggaggagttggccgaggaaaagatatctccaccatcacaggtcatcgtgggaaagacatctctactattttggatgaagaaagaaaagaaaataaacgaccccagagggcagctgctgctcgaaggaagaaacgccggcgattaaatgatctggacagtgatagcaacctggatgaagaagagagcgaggatgaattcaagatcagtgatggatctcaagatgagtttgttgtgtctgatgaaaacccagatgaaagtgaagaagatccgccatctaatgatgacagtgacactgacttttgtagccgtagactgaggcgacacccctctcggccaatgaggcagagcaggcgtttgcgaagaaagaccccaaagaaaaaatattccgatgatgatgaagaggaggaatctgaggagaatagtagagactctgaaagtgacttcagtgatgattttagtgatgattttgtagaaactcggcgaaggcggtcaaggagaaatcagaaaagacaaattaactacaaagaagactcagaaagtgacggttcccagaagagtttgcgacgtggtaaagaaataaggcgagtacacaagcgaagactttccagctcagagagtgaagagagctatttgtccaagaactctgaagatgatgagctagctaaagaatcaaagcggtcagttcgaaagcggggccgaagcacagacgagtattcagaagcagatgaggaggaggaggaagaggaaggcaaaccatcccgcaaacggctacaccggattgagacggatgaggaggagagttgtgacaatgctcatggagatgcaaatcagcctgcccgtgacagccagcctagggtcctgccctcagaacaagagagcaccaagaagccctaccggatagaaagtgatgaggaagaggactttgaaaatgtaggcaaagtggggagcccattggactatagcttagtggacttaccttcaaccaatggacagagccctggcaaagccattgagaacttgattggcaagcctactgagaagtctcagacccccaaggacaacagcacagccagtgcaagcctagcctccaatgggacaagtggtgggcaggaggcaggagcaccagaagaggaggaagatgagcttttgagagtgactgaccttgttgattatgtctgtaacagtgaacagttataagactttttttccatttttgtgctaatttattccacggtagctctcacaccagcgggccagttattaaaagctgtttaatttttcctagaaaactccactacagaatgacttttagaagaaaaatttcaacaaatcctgaagtctttctgtgaagtgaccagttctgaactttgaagataaataattgctgtaaattccttttgattttctttttccaggttcatggtccttggtaatttcattcatggaaaaaaatcttattataataacaacaaagatttgtatatttttgactttatatttcctgagctctcctgactttgtgaaaaagggtggatgaaaatgcattccgaatctgtgagggcccaaaacagaatttaggggtgggtgaaagcacttgtgctttagctttttcatattaaatatatattatatttaaacattcatggcatagatgatgatttacagacaatttaaaagttcaagtctgtactgttacagtttgagaattgtagataacatcatacataagtcatttagtaacagcctttgtgaaatgaacttgtttactattggagataaccacacttaataaagaagagacagtgaaagtaccatcataattaacctaaatttttgttatagcagagtttcttgtttaaaaaaaaataaaatcatctgaaaagcaaaaa

SEQ ID NO: 36 - DNA, 2288 bp, Homo sapiens
  source 1..2288: mol_type = other DNA
cgcgcgctgcagtgccttccccacctcggccccgcccgcccccgccgagccgagcaccagggcggcggcggcggcggcggcggcggcggcggctggagcagcccgggaggaggaggcggcgagaatggcagcggcgtcgtgggcgcggcggagatgagcgcccgcgaccccgggcccagggcggcacagccggagtgggcgggggtcccgatgcaggcccgaggggggccatggggcaggtcctgccggtcttcgcccactgcaaagaagctccgtctacagcctcctcaactcctgattccacagaaggagggaacgacgactctgattttcgagagctgcacacagcccgggaattctcagaggaggacgaggaggagaccacgtcgcaggactggggcaccccccgggagctgaccttctcctacatcgcctttgatggtgtagtgggctccgggggccgcagggattcaactgcccgccgcccccgcccccagggccgctcagtctcggaaccacgagaccagcaccctcagcccagcctgggcgacagcttggagagcatccccagcctgagccaatccccggagcctggacgacggggtgatcctgacaccgcgcctccatccgagcgccctctggaagacctgaggcttcggttggaccatctgggctgggtggcccggggaacgggatccggggaggactcttccaccagcagctccaccccgctggaagacgaagaaccccaagaacccaacagattggagacaggagaagctggggaagaactggacctacgactccgacttgctcagccctcatcgcccgaggtcttgactccccagctcagtccgggctctgggacaccccaggccggtactccgtccccatcccgatcgcgagattcgaactctgggcccgaagagccattgctggaagaggaagaaaagcagtgggggccactggagcgagagccagtaaggggacagtgcctcgatagcacggaccaattagaattcacggtggagccacgccttctaggaacagctatggaatggttaaagacatcattgcttttggctgtttacaagacggttccaattttggaattgtccccacctctgtggacagccattggctgggtccaaaggggccccaccccccctactcctgtcctccgggttctactgaagtgggcaaaatccccgagaagcagcggtgtccccagcctctcactcggagccgatatggggagtaaagtggcggacctgctgtactggaaggacacgaggacgtcaggagtggtcttcacaggcctgatggtctccctcctctgcctcctgcactttagcatcgtgtccgtggccgcgcacttggctctgttgctgctctgcggcaccatctctctcagggtttaccgcaaagtgctgcaggccgtgcaccggggggatggagccaaccctttccaggcctacctggatgtggacctcaccctgactcgggagcagacggaacgtttgtcccaccagatcacctcccgcgtggtctcggcggccacgcagctgcggcacttcttcctggtagaagacctcgtggattccctcaagctggccctcctcttctacatcttgaccttcgtgggtgccatcttcaatggtttgactcttctcattctgggagtgattggtctattcaccatccccctgctgtaccggcagcaccaggctcagatcgaccaatatgtggggttggtgaccaatcagttgagccacatcaaagctaagatccgagctaaaatcccagggaccggagccctggcctctgcagcagccgcagtctccggatccaaagccaaagccgaatgagaacggtgtctctgcccgcaggacgcctgcccccagcccccgcagccctctggccccctccatctcttgtccgttcccacccacccccctcctcggcccgagccttttcccggtgggtgtcaggatcactcccactagggactctgcgctaattacctgagcgaccaggactacatttcccaagaggctctgctccaggagtccaggaaagacgaggcaccttggccgcggggcctgctgggacttgtagttgcctagacagggcaccaccctgcacttccggacccgccgctggaggcgccgtgaggcgttggtgtctcctggatgctactagccccaacgccggggctttgcatggggcccaggggaggcctgagcttggatttacactgtaataaagactcctgtggaaaacccgag

SEQ ID NO: 37 - DNA, 1700 bp, Homo sapiens
  source 1..1700: mol_type = other DNA
agcaggactcagaggggagagttggaggaaaaaaaaaggcagaaaagggaaagaaagaggaagagagagagagagtgagaggagccgctgagcccaccccgatggccgcggacgaagttgccggaggggcgcgcaaagccacgaaaagcaaactttttgagtttctggtccatggggtgcgccccgggatgccgtctggagcccggatgccccaccagggggcgcccatgggccccccgggctccccgtacatgggcagccccgccgtgcgacccggcctggcccccgcgggcatggagcccgcccgcaagcgagcagcgcccccgcccgggcagagccaggcacagagccagggccagccggtgcccaccgcccccgcgcggagccgcagtgccaagaggaggaagatggctgacaaaatcctccctcaaaggattcgggagctggtccccgagtcccaggcttacatggacctcttggcatttgagaggaaactggatcaaaccatcatgcggaagcgggtggacatccaggaggctctgaagaggcccatgaagcaaaagcggaagctgcgactctatatctccaacacttttaaccctgcgaagcctgatgctgaggattccgacggcagcattgcctcctgggagctacgggtggaggggaagctcctggatgatcccagcaaacagaagcggaagttctcttctttcttcaagagtttggtcatcgagctggacaaagatctttatggccctgacaaccacctcgttgagtggcatcggacacccacgacccaggagacggacggcttccaggtgaaacggcctggggacctgagtgtgcgctgcacgctgctcctcatgctggactaccagcctccccagttcaaactggatccccgcctagcccggctgctggggctgcacacacagagccgctcagccattgtccaggccctgtggcagtatgtgaagaccaacaggctgcaggactcccatgacaaggaatacatcaatggggacaagtatttccagcagatttttgattgtccccggctgaagttttctgagattccccagcgcctcacagccctgctattgccccctgacccaattgtcatcaaccatgtcatcagcgtggacccttcagaccagaagaagacggcgtgctatgacattgacgtggaggtggaggagccattaaaggggcagatgagcagcttcctcctatccacggccaaccagcaggagatcagtgctctggacagtaagatccatgagacgattgagtccataaaccagctcaagatccagagggacttcatgctaagcttctccagagaccccaaaggctatgtccaagacctgctccgctcccagagccgggacctcaaggtgatgacagatgtagccggcaaccctgaagaggagcgccgggctgagttctaccaccagccctggtcccaggaggccgtcagtcgctacttctactgcaagatccagcagcgcaggcaggagctggagcagtcgctggttgtgcgcaacacctaggagcccaaaaataagcagcacgacggaactttcagccgtgtcccgggccccagcattttgccccgggctccagcatcactcctctgccaccttggggtgtggggctggattaaaagtcattcatctgacaaaaaaaaaaaaaaaaaa

SEQ ID NO: 38 - DNA, 1935 bp, Homo sapiens
  source 1..1935: mol_type = other DNA
acaatagcgactcactggacccagcccttagcaacggcctggcgacggtttccctgctgctgcagcccccgtcggctcctcttttccagtcctccactgccggggctgggcccggccgcgggaaggaccgaaggggatacagcgtgtccctgcggcggctgcaagaggactaagcatggatggcagccggagagtcagagcaacctctgtccttcccagatatggtccaccgtgcctatttaaaggacacttgagcaccaaaagtaatgctgcagtagactgctcggttccagtaagcgtgagtaccagcataaagtatgcagaccaacaacgaagagagaaactcaaaaaggaattagcacaatgtgaaaaagagttcaaattaactaaaactgcaatgcgagccaattataaaaataattccaagtcactttttaataccttacaaaagccctcaggcgaaccgcaaattgaggatgacatgttaaaagaagaaatgaatggattttcatcctttgcaaggtcactagtaccctcttcagagagactacacctaagtctacataaatccagtaaagtcatcacaaatggtcctgagaagaactccagttcctccccgtccagtgtggattatgcagcctccgggccccggaaactgagctctggagccctgtatggcagaaggcccagaagcacattcccaaattcccaccggtttcagttagtcatttcgaaagcacccagtggggatcttttggataaacattctgaactcttttctaacaaacaattgccattcactcctcgcactttaaaaacagaagcaaaatctttcctgtcacagtatcgctattatacacctgccaaaagaaaaaaggattttacagatcaacggatagaagctgaaacccagactgaattaagctttaaatctgagttggggacagctgagactaaaaacatgacagattcagaaatgaacataaagcaggcatctaattgtgtgacatatgatgccaaagaaaaaatagctcctttacctttagaagggcatgactcaacatgggatgagattaaggatgatgctcttcagcattcctcaccaagggcaatgtgtcagtattccctgaagcccccttcaactcgtaaaatctactctgatgaagaagaactgttgtatctgagtttcattgaagatgtaacagatgaaattttgaaacttggtttattttcaaacaggtttttagaacgactgttcgagcgacatataaaacaaaataaacatttggaggaggaaaaaatgcgccacctgctgcatgtcctgaaagtagacttaggctgcacatcggaggaaaactcggtaaagcaaaatgatgttgatatgttgaatgtatttgattttgaaaaggctgggaattcagaaccaaatgaattaaaaaatgaaagtgaagtaacaattcagcaggaacgtcaacaataccaaaaggctttggatatgttattgtcggcaccaaaggatgagaacgagatattcccttcaccaactgaatttttcatgcctatttataaatcaaagcattcagaaggggttataattcaacaggtgaatgatgaaacaaatcttgaaacttcaactttggatgaaaatcatccaagtatttcagacagtttaacagatcgggaaacttctgtgaatgtcattgaaggtgatagtgaccctgaaaaggttgagatttcaaatggattatgtggtcttaacacatcaccctcccaatctgttcagttctccagtgtcaaaggcgacaataatcatgacatggagttatcaactcttaaaatcatggaaatgagcattgaggactgccctttggatgtttaatcttcattaataaatacctcaaatggccagtaactcaaaaaaaaaaaaaaaaaaaa

SEQ ID NO: 39 - DNA, 4343 bp, Homo sapiens
  source 1..4343: mol_type = other DNA
tggtcatcgcacggcggcagctcctcacctggatttagaagagctggcgtccccgcccgcccaagcctttaaactctcgtctgccagaacccgccaactctccaggcttagggccagtttccgcgattctaagagtaattgcgtgggcacctgtgctggggccaggcgcaaagaagggagttggtctgcgcgaagatcgtcaacctgctaacagaccgcacatgcactttgcaccgaccatctacgtctcagtctggaggttgcgcactttggctgctgacgcgctggtggtgcctattaatcatttaccagtccagagccgcgccagttaatggctgtgccgtgcggtgctcccacatcctggcctctcctctccacggtcgcctgtgcccgggcaccccggagctgcaaactgcagagcccaggcaaccgctgggctgtgcgccccgccggcgccggtaggagccgcgctccccgcagcggttgcgctctacccggaggcgctgggcggctgtgggctgcaggcaagcggtcgggtggggagggagggcgcaggcggcgggtgcgcgaggagaaagccccagccctggcagccccactggcccccctcagctgggatgttccccaatggcaccgcctcctctccttcctcctctcctagccccagcccgggcagctgcggcgaaggcggcggcagcaggggccccggggccggcgctgcggacggcatggaggagccagggcgaaatgcgtcccagaacgggaccttgagcgagggccagggcagcgccatcctgatctctttcatctactccgtggtgtgcctggtggggctgtgtgggaactctatggtcatctacgtgatcctgcgctatgccaagatgaagacggccaccaacatctacatcctaaatctggccattgctgatgagctgctcatgctcagcgtgcccttcctagtcacctccacgttgttgcgccactggcccttcggtgcgctgctctgccgcctcgtgctcagcgtggacgcggtcaacatgttcaccagcatctactgtctgactgtgctcagcgtggaccgctacgtggccgtggtgcatcccatcaaggcggcccgctaccgccggcccaccgtggccaaggtagtaaacctgggcgtgtgggtgctatcgctgctcgtcatcctgcccatcgtggtcttctctcgcaccgcggccaacagcgacggcacggtggcttgcaacatgctcatgccagagcccgctcaacgctggctggtgggcttcgtgttgtacacatttctcatgggcttcctgctgcccgtgggggctatctgcctgtgctacgtgctcatcattgctaagatgcgcatggtggccctcaaggccggctggcagcagcgcaagcgctcggagcgcaagatcaccttaatggtgatgatggtggtgatggtgtttgtcatctgctggatgcctttctacgtggtgcagctggtcaacgtgtttgctgagcaggacgacgccacggtgagtcagctgtcggtcatcctcggctatgccaacagctgcgccaaccccatcctctatggctttctctcagacaacttcaagcgctctttccaacgcatcctatgcctcagctggatggacaacgccgcggaggagccggttgactattacgccaccgcgctcaagagccgtgcctacagtgtggaagacttccaacctgagaacctggagtccggcggcgtcttccgtaatggcacctgcacgtcccggatcacgacgctctgagcccgggccacgcaggggctctgagcccgggccacgcaggggccctgagccaaaagagggggagaatgagaagggaaggccgggtgcgaaagggacggtatccagggcgccagggtgctgtcgggataacgtggggctaggacactgacagcctttgatggaggaacccaagaaaggcgcgcgacaatggtagaagtgagagctttgcttataaactgggaaggctttcaggctacctttttctgggtctcccactttctgttccttcctccactgcgcttactcctctgaccctccttctattttccctaccctgcaacttctatcctttcttccgcaccgtcccgccagtgcagatcacgaactcattaacaactcattctgatcctcagcccctccagtcgttatttctgtttgtttaagctgagccacggataccgccacgggtttccctcggcgttagtccctagccgcgcggggccgctgtccaggttctgtctggtgcccctactggagtcccgggaatgaccgctctccctttgcgcagccctaccttaaggaaagttggacttgagaaagatctaagcagctggtcttttctcctactcttgggtgaaggtgcatctttccctgccctcccctgtccccctctcgccgcccgcccgccaccaccactctcactccacccagagtagagccaggtgcttagtaaaataggtcccgcgcttcgaactccaggctttctggagttcccacccaagccctcctttggagcaaagaaggagctgagaacaagccgaatgaggagtttttataagattgcggggtcggagtgtgggcgcgtaataggaatcaccctcctactgcgcgttttcaaagaccaagcgctgggcgctcccgggccgcgcgtctgcgttaggcagggcagggtagtgcagggcacaccttccccggggttcggggttcggggttcggttgcagggctgcagcccgccttggctttctccctcacccaagtttccggaggagccgacctaaaagtaacaatagataaggtttcctgctccagtgtatctcaaaagaccgggcgccaggggcgggggacctagggcgacgtcttcagagtccgccagtgttggcggtgtcgccgcaacctgcaggctcccgagtggggcctgcctggtctctagagggttgctgcctttcaagcggtgcctaagaagttattttcttgtttaacatatatatttattaatttatttgtcgtgttggaaaatgtgtctctgctttccttttctctgcttgcctagccccaggtcttttctttgggaccctgggggcgggcatggaagtggaagtaggggcaagctcttgccccactccctggccatctcaacgcctctcctcaatgctgggccctcttatctcatcctttcctctagcttttctatttttgattgtgttgagtgaagtttggagatttttcatacttttcttactatagtctcttgtttgtcttattaggataatacataaatgataatgtgggttatcctcctctccatgcacagtggaaagtcctgaactcctggctttccaggagacatatataggggaacatcaccctatatataatttgagtgtatatatatttatatatatgatgtggacatatgtatacttatcttgctccattgtcatgagtccatgagtctaagtatagccactgatggtgacaggtgtgagtctggctggaacactttcagtttcaggagtgcaagcagcactcaaacctggagctgaggaatctaattcagacagagactttaatcactgctgaagatgcccctgctccctctgggttccagcagaggtgattcttacatatgatccagttaacatcatcactttttttgaggacattgaaagtgaaataatttgtgtctgtgtttaatattaccaactacattggaagcctgagcagggcgaggaccaataattttaattatttatatttcctgtattgctttagtatgctggcttgtacatagtaggcactaaatacatgtttgttggttgattgtttaagccagagtgtattacaacaatctggagatactaaatctggggttctcaggttcactcattgacatgatatacaatggttaaaatcactattgaaaaatacgttttgtgtatatttgcttcaacaactttgtgctttcctgaaagcagtaaccaagagttaagatatccctaatgttttgcttaaactaatgaacaaatatgctttgggtcataaatcagaaagtttagatctgtcccttaataaaaatatatattactactcctttggaaaatagatttttaatggttaagaactgtgaaatttacaaatcaaaatcttaatcattatccttctaagaggatacaaatttagtgctcttaacttgttaccattgtaatattaactaaataaacagatgtattatgctgttaaaaaaaaaaaaaaaaaaaaaaaaaaaaaaaaaaaaaaaaaaaaaaaaaaaa

SEQ ID NO: 40 - DNA, 4171 bp, Homo sapiens
  source 1..4171: mol_type = other DNA
ctgcatctctccctctcacccgtgtctcctctcctctctttccttctcgtcttctccctgtcacgcatctctcatcactccccctcattctgcctttcctcctactcacggtctcctctccctctccctctctctctctccccctccctctttctctctctctctctttctccacctcctcccgaccccctttcccctctatttctattggcttctgtgtcccttgctcccctcttctcttcctcaccctgggaagcttctcccccctatccttgcccctgcccccccaggatgtgtcctggagatggggggtgacgtaccaggctctggttgggaagtcagggccggagaccagatgggagaggctctgtggacagccgtggccgagggcctgggagggaacctgagcccgcaagcggtctagaagtgggtgccttgtggggaccctagttaggagtgccctgggggcacctggggactgggcagggagaggggacagcagaatgataaccagcctggcggcaaggagggaagccctcaccccatgggcaggcaaatagctgactgctgaccaccctcccctcagccatggacatgcttcatccatcatcggtgtccacgacctcagaacctgagaatgcctcctcggcctggcccccagatgccaccctgggcaacgtgtcggcgggcccaagcccggcagggctggccgtcagtggcgttctgatccccctggtctacctggtggtgtgcgtggtgggcctgctgggtaactcgctggtcatctatgtggtcctgcggcacacggccagcccttcagtcaccaacgtctacatcctcaacctggcgctggccgacgagctcttcatgctggggctgcccttcctggccgcccagaacgccctgtcctactggcccttcggctccctcatgtgccgcctggtcatggcggtggatggcatcaaccagttcaccagcatattctgcctgactgtcatgagcgtggaccgctacctggccgtggtacatcccacccgctcggcccgctggcgcacagctccggtggcccgcacggtcagcgcggctgtgtgggtggcctcagccgtggtggtgctgcccgtggtggtcttctcgggagtgccccgcggcatgagcacctgccacatgcagtggcccgagccggcggcggcctggcgagccggcttcatcatctacacggccgcactgggcttcttcgggccgctgctggtcatctgcctctgctacctgctcatcgtggtgaaggtgcgctcagctgggcgccgggtgtgggcaccctcgtgccagcggcggcggcgctccgaacgcagggtcacgcgcatggtggtggccgtggtggcgctcttcgtgctctgctggatgcccttctacgtgctcaacatcgtcaacgtggtgtgcccactgcccgaggagcctgccttctttgggctctacttcctggtggtggcgctgccctatgccaacagctgtgccaaccccatcctttatggcttcctctcctaccgcttcaagcagggcttccgcagggtcctgctgcggccctcccgccgtgtgcgcagccaggagcccactgtggggcccccggagaagactgaggaggaggatgaggaggaggaggatggggaggagagcagggaggggggcaaggggaaggagatgaacggccgggtcagccagatcacgcagcctggcaccagcgggcaggagcggccgcccagcagagtggccagcaaggagcagcagctcctaccccaagaggcttccactggggagaagtccagcacgatgcgcatcagctacctgtaggggcctggggaaagccaggatggcccgaggaagaggcagaagccgtgggtgtgcctagggcctacttcccaaggtgccacaggcccatgatgggatgttgaggggcctggactttgatgctattgctgccaggtcttgctgtgtgaccttgggtaggttgcttctactctctgggccttgttttctcctctgtgactcagggataggagtcatcagcctggatgagctatgtcagatgagaggtttggagggcactgttgctgggctgacctggctgagcaggcaaaaggtgggtgcagactggcctccccccagggatggagtgtcttggggcatcaactagaatcttggccctcagagggataaaccaaggccaggatttcttgggctcagagtcaggaacacaggagctgctgggggctgggctggaaacctaaacagaagaaagcctaacccggtgggaggagtggggcagaaatggtcaggccccagatcagctccctcccctcgactgtgaggccttggaccagctctgctcctctctaggcctcaggcttcacctgggtaaaacccaacaacctctacacccttttggcccaggcagtcaatgctggaggtcctgtgctcctggacgggaagagcaggtgaatttcctgctcatggaagcgaatgaagtccagcttcagggtctctcactgcctgggcttttgcaaggccctgcatctacttttgtacttgtcattttgtattcgttttcttaaagagggacctcgaactgcataagcttaggccacccaaagcctggctctgcccctgctgaggtcagccacccaatccccaaggaagctcatgttgggtcttatggctggagtaggggcccccgggggttcccaggtcttttgagggcttccaggcacctccttgtaggaagggccatccctgttcctctccttgtgacccatattctcccttcctggagaccgagacagggacccagcccatgaggactggcatggaaaggcagagtgtctgaagagcgctgtgaggagaaggaagaggaagggagaagaggaagaggaaggagaaggaagaggaagacaagggggaaaggggaggatgaggagggggaaggagaagtacagatctgtttcctggagccgtctttggcccccctgggctgagctcagtggtagcatctgtgaacctgagttgccgacaacagccccacccaaccagtactgagggaaggacacgatcagggtggaacagccagggtgcaatggcaaatgcacagagtacagacaggcacagggcctgcgtccctgaggggcctcagagtgctgccaagagggctcaggccttaataaagccctagggtggagctggctaccagggacattgggaggactggggagctccctccccatgctctatcatcctggagactacaggtcgggaggcccagggaagacaagaagaggctgaagtgggactgtggagggggaccatggggagcagccaccatccaaggctgggcctagactccctcccagagatggtccctcagagctgtggtgaggctggccctgggagggtgagacccccggtgaaatccttccgcttccccaccccttgcagagggcaggggtcctcagggaaagcacaggaaccagacttttggagacttggatcttcagcacacctcagggtcctgggctggcattggccttccgggcctcaatttccccatcaacaaatggagatgaatcccagcttggctgcctcctgggatctaacgagaaaatgagtcatgtgaggtaacttccaggctcactgcaatgggtacggtggggtgtatcagattataaagtgggggtgccctcctcacccccaggcttggcctatacccccctctccatcaagtggcctctctgtgtctgtcctttggggtgaggacactgtaggccatgagaaatgggcagttggggggtcagaggccaagggttagggaggcagggcttggggagagtgtgggaccatcagaagagaaggaagtttacaaaaccacattttgtgtggagatggaggctggaggcccggccctgggacttggtctggggtttcttgaggaagatctgagggtccaagggaggaaggatgccctggccttctggccttctctggctgatcctgccttcttgctgcctaggacaggagagtaatgtcctagaatggtccctgggaggccagttaggaaaccctttgctgcttctgtctctagctcttgtcaataaagacggtgacacctgaaaaaaaaaaaaaaaaa

SEQ ID NO: 41 - DNA, 1258 bp, Homo sapiens
  source 1..1258: mol_type = other DNA
ccgagctctctggcgcagcgctagctccgccgcgctcagctgccctgcgccggcacccctggtcatgagcgccccctcgacgctgccccccgggggcgaggaagggctggggacggcctggccctctgcagccaatgccagtagcgctccggcggaggcggaggaggcggtggcggggcccggggacgcgcgggcggcgggcatggtcgctatccagtgcatctacgcgctggtgtgcctggtggggctggtgggcaacgccctggtcatcttcgtgatccttcgctacgccaagatgaagacggctaccaacatctacctgctcaacctggccgtagccgacgagctcttcatgctgagcgtgcccttcgtggcctcgtcggccgccctgcgccactggcccttcggctccgtgctgtgccgcgcggtgctcagcgtcgacggcctcaacatgttcaccagcgtcttctgtctcaccgtgctcagcgtggaccgctacgtggccgtggtgcaccctctgcgcgcggcgacctaccggcggcccagcgtggccaagctcatcaacctgggcgtgtggctggcatccctgttggtcactctccccatcgccatcttcgcagacaccagaccggctcgcggcggccaggccgtggcctgcaacctgcagtggccacacccggcctggtcggcagtcttcgtggtctacactttcctgctgggcttcctgctgcccgtgctggccattggcctgtgctacctgctcatcgtgggcaagatgcgcgccgtggccctgcgcgctggctggcagcagcgcaggcgctcggagaagaaaatcaccaggctggtgctgatggtcgtggtcgtctttgtgctctgctggatgcctttctacgtggtgcagctgctgaacctcttcgtgaccagccttgatgccaccgtcaaccacgtgtcccttatccttagctatgccaacagctgcgccaaccccattctctatggcttcctctccgacaacttccgccgattcttccagcgggttctctgcctgcgctgctgcctcctggaaggtgctggaggtgctgaggaggagcccctggactactatgccactgctctcaagagcaaaggtggggcagggtgcatgtgccccccactcccctgccagcaggaagccctgcaaccagaacccggccgcaagcgcatccccctcaccaggaccaccaccttctgaggagcccttcccctacccaccctgcgt

SEQ ID NO: 42 - DNA, 2674 bp, Homo sapiens
  source 1..2674: mol_type = other DNA
atgcctgcatgtgctggttcagggactcaccaccctggcgtcctcccttcttctcttgcagagcctgacgcaccccagggctgccgccatggagcccctgttcccagcctccacgcccagctggaacgcctcctccccgggggctgcctctggaggcggtgacaacaggacgctggtggggccggcgccctcggcaggggcccgggcggtgctggtgcccgtgctgtacctgctggtgtgtgcggccgggctgggcgggaacacgctggtcatctacgtggtgctgcgcttcgccaagatgaagaccgtcaccaacatctacattctcaacctggcagtggccgacgtcctgtacatgctggggctgcctttcctggccacgcagaacgccgcgtccttctggcccttcggccccgtcctgtgccgcctggtcatgacgctggacggcgtcaaccagttcaccagtgtcttctgcctgacagtcatgagcgtggaccgctacctggcagtggtgcacccgctgagctcggcccgctggcgccgcccgcgtgtggccaagctggcgagcgccgcggcctgggtcctgtctctgtgcatgtcgctgccgctcctggtgttcgcggacgtgcaggagggcggtacctgcaacgccagctggccggagcccgtggggctgtggggcgccgtcttcatcatctacacggccgtgctgggcttcttcgcgccgctgctggtcatctgcctgtgctacctgctcatcgtggtgaaggtgagggcggcgggcgtgcgcgtgggctgcgtgcggcggcgctcggagcggaaggtgacgcgcatggtgttggtggtggtgctggtgtttgcgggatgttggctgcccttcttcaccgtcaacatcgtcaacctggccgtggcgctgccccaggagcccgcctccgccggcctctacttcttcgtggtcatcctctcctacgccaacagctgtgccaaccccgtcctctacggcttcctctctgacaacttccgccagagcttccagaaggttctgtgcctccgcaagggctctggtgccaaggacgctgacgccacggagccgcgtccagacaggatccggcagcagcaggaggccacgccacccgcgcaccgcgccgcagccaacgggcttatgcagaccagcaagctgtgagagtgcaggcggggggtgggcggccccgtgtcacccccaggagcggaggttgcactgcggtgacccccacccatgacctgccagtcaggatgctccccggcggtggtgtgaggacagagctggctgaagccaggctggggtagacacagggcagtaggttccccaccgtgaccgaccatcccctctaaccgtctgccacacagcgggggctcccgggaggtaggggaggtggccagaccggtggggggctccgccatgccgtgcaagtgctcagggccgcctcaccctccatctggccccagcccatgccggccttccctctggggagcgacttttccagaaggccggccaggcgagagggtcttcctgacggcggagctgacctgcccggcccaccagctgcatgtcagctccgagccaccgggtccccgtccaaggctgctctgctaagttaaagacacccgaaagcgcttgactcaggtccccggagtccctggccagggccccagcccctcgcttgccctgcactgtgtggactctggggatgcaggtgtaaggggagtgtggctgggcagcccctggtcagccagggtcacgcctgtcctgggggccccaccctgctgcccgacaccccccatgggaggctgcgggcggcagttgctgtctcagagaggggagtgtgggggcttgggcgctggcctagccaggggcgaggtggggaggcggctggtgcagaggagagctgggggctgaggttggggtgaaggctgcagccctccaggctgctgggggtgcagatggctgtgccgtgctgagattggctctgtctggaggggtccagtgtggggtgcctgagggcactagggagaggtgctcctgctgcaggaggacctgagggtcagggcttggagaggacagggaacctgcggccgtctcttctgctttggggcaggggctctggcccgggagagggaacggggacaggagcagaggacggtcatccaggcgcagcggggagctgctccccaggccacagcagacagcactgctgagaggcagcggccgcgcgggtgacgcaaatggcaggccctgggaatcccgccgcctcccacctagaattgtcctacctcccccaccccaaacaccagcttttcctggcgccccaggcccagaacgtgggcccagagagccttgctggggtctctggggcaccttggccttgctctgaggctggaaggagaaggaccagggtgcggcatcactcggcctcagggacccctctgccctgcccagcactggccccgacccgtgctcccgccgtctgcccagagcaggacctcaacctcctggagggcacagggagcggctgagtgggcacaaatcctggcaggagaaaggcccaggctgaggccaggcctgggaaacatccaagcagtgaggacacgcgtgtttgacaactgctcccctgaataaatgcgaggataaatgttt

SEQ ID NO: 43 - DNA, 4427 bp, Homo sapiens
  source 1..4427: mol_type = other DNA
cccccggcggagccagctgctgctcttcggtgctggccccggtgccggccccgttgcccagggaacaggctcccggcagcccccgcggcccggagtccatcccgcctcctccggcccggcggggccgacgagtccggaggggctgccgcgggagcccccaggtttccctagatgacaaataaacattccttttcctgcgtgaagatagtctgtggaaaccttggccatggcatcgatatcagagcctgttacattcagagagttctgcccgttgtactatctcctcaatgccattccgacaaagatccagaagggtttccgctctatcgtggtctatctcacggccctcgacaccaacggggactacatcgcggtgggcagcagcatcggcatgctctatctgtactgccggcacctcaaccagatgaggaagtacaactttgaggggaagacggaatctatcactgtggtgaagctgctgagctgctttgatgacctggtggcagcaggcacagcctctggcagggttgcagtttttcaacttgtatcttcattgccagggagaaataaacagcttcggagatttgatgtcactggtattcacaaaaatagcattacagctctggcttggagccccaatggaatgaaattgttctctggagatgacaaaggcaaaattgtttattcttctctggatctagaccaggggctctgtaactcccagctggtgttggaggagccatcttccattgtgcagctggattatagccagaaagtgctgctggtctctactctgcaaagaagtctgctcttttacactgaagaaaagtctgtaaggcaaattggaacacaaccaaggaaaagtactgggaaatttggtgcttgttttataccaggactctgtaagcaaagtgatctaaccttgtatgcgtcacggcccgggctccggctatggaaggctgatgtccacgggactgttcaagccacgtttatcttaaaagatgcttttgccgggggagtcaagccttttgaactgcacccgcgtctggaatcccccaacagtggaagttgcagcttacctgagaggcacctggggcttgtttcatgtttctttcaagaaggctgggtgctgagttggaatgaatatagtatctatctcctagacacagtcaaccaggccacagttgctggtttggaaggatccggtgatattgtgtctgtttcgtgcacagaaaatgaaatatttttcttgaaaggagataggaacattataagaatttcaagcaggcctgaaggattaacatcaacagtgagagatggtctggagatgtctggatgctcagagcgtgtccacgtgcagcaagcggagaagctgccaggggccacagtttctgagacgaggctcagaggctcttccatggccagctccgtggccagcgagccaaggagcaggagcagctcgctcaactccaccgacagcggctccgggctcctgccccctgggctccaggccacccctgagctgggcaagggcagccagcccctgtcacagagattcaacgccatcagctcagaggactttgaccaggagcttgtcgtgaagcctatcaaagtgaaaaggaagaagaagaagaagaagacagaaggtggaagcaggagcacctgtcacagctccctggaatcgacaccctgctccgaatttcctggggacagtccccagtccttgaacacagacttgctgtcgatgacctcaagtgtcctgggcagtagcgtggatcagttaagtgcagagtctccagaccaggaaagcagcttcaatggtgaagtgaacggtgtcccacaggaaaatactgaccccgaaacgtttaatgtcctggaggtgtcaggatcaatgcctgattctctggctgaggaagatgacattagaactgaaatgccacactgtcaccatgcacatgggcgggagctgctcaatggagcgagggaagatgtgggaggcagtgatgtcacgggactcggagatgagccgtgtcctgcagatgatggaccaaatagcacacagttacccttccaagaacaggacagctctcctggggcgcatgatggggaagacatccaacccattggcccccaaagcactttttgtgaagtccccctcctgaactcactcactgtgccttccagcctcagctgggccccaagtgctgaacagtggctgcctgggaccagagctgatgaaggcagccccgtggagcccagccaagagcaggacatcctaaccagcatggaggcctctggccacctcagcacaaatctctggcatgctgtcactgatgatgacacaggtcagaaagaaatacccatttctgaacgtgtcttggggagtgtgggaggacagctgactccggtctctgccttggcagccagcactcacaagccctggcttgagcagcctccacgggatcagacattgacgtccagcgatgaggaggacatctatgcccacgggcttccttcttcatcctcagagacgagtgtgacagagctcggacctagttgctcccagcaggacctgagccggctgggtgcagaggacgccgggctgctcaagccagatcagtttgcagaaagctggatgggctactcgggtcccggctatggcatcctcagcttggtggtctccgagaagtatatctggtgcctggactacaaaggcggcctgttctgcagcgcgttgccgggcgccgggctgcgctggcagaagtttgaagatgctgtccagcaggtggcagtctcgccctcaggagcccttctctggaagattgaacagaaatctaaccgggcttttgcttgtgggaaagtcaccatcaaggggaagcggcactggtacgaagccctgccccaggcagtgtttgtggccctgagcgatgacacggcctggatcatcaggaccagtggggacctatacttgcagacaggtctgagcgtggatcgcccttgtgccagagccgtaaaggtggactgtccctacccgctgtcccagatcacagcccggaacaatgtggtgtgggcgctgacagagcagagggccctcctgtaccgggagggcgtgagcagcttctgtccggaaggcgagcagtggaagtgtgacattgtcagcgaaaggcaagctttagaacccgtctgcataacgctcggggatcagcagactctctgggccctggacatccatgggaacctgtggttcagaactggcattatttccaagaagccccaaggagatgacgaccattggtggcaagtgagcatcacggactatgtggtgtttgaccagtgcagcttatttcagacgataatccatgccactcactcggtggccacagcagcccaagcccccgtagaaaaggtggcagataagctgcgcatggcgttttggtcccagcagcttcagtgccagccaagccttctcggggtcaataacagcggtgtctggatctcctcgggcaagaatgaattccacgtcgctaagggaagtctcataggcacctactggaatcatgtggttccccgtgggacagcttctgctacaaaatgggcctttgtgttggcttctgcagctcccacgaaggaaggaagcttcctgtggctgtgccagagcagcaaggacctgtgcagcgtcagcgcccagagcgcacagtcgcggccctccacggtgcagctgcctcccgaagccgagatgcgcgcctatgccgcctgccaggatgcgctgtgggcgctggacagcctcggccaggtgttcatcaggacgctctccaagagctgccccacgggcatgcactggaccaggctggacctctcccagctaggagctgtaaaattgacaagcttggcatgtggaaatcagcacatctgggcctgtgattccaggggtggagtttacttccgtgtagggactcagcctctcaatcccagtctcatgcttccagcctggataatgattgagccacctgtccaggtaagcagaagttagctggtggaactcactcttcagtaagacagaaactgtgaggatgctggtactgggaaaaaggatctgcacagcctctagaggcctcccagcaaatgcggggagccatgcccccagggtctacacactctcgttcatcaacatcacaactggaattcgggatttgtgaagtttagagctgaacagactgttacagattatgagtcaacacgtatattttctctttcaaaataataatatttcgtttttgactttttactaagtgaatattattttttaaatctgcctatatattggaacctctattttataataataatgataataaatcagtacccagaagtataaagaaggtaaaagttactttgaaaaaaaaaaaaaaaaaaaaaaaaaaaaaaaa

SEQ ID NO: 44 - DNA, 1815 bp, Homo sapiens
  source 1..1815: mol_type = other DNA
ttttagagaattactccaaattcatcatgattgaagacaataaggagaacaaagaccattccttagaaaggggaagagcaagtctcattttttccttaaagaatgaagttggaggacttataaaagccctgaaaatctttcaggagaagcatgtgaatctgttacatatcgagtcccgaaaatcaaaaagaagaaactcagaatttgagatttttgttgactgtgacatcaacagagaacaattgaatgatatttttcatctgctgaagtctcataccaatgttctctctgtgaatctaccagataattttactttgaaggaagatggtatggaaactgttccttggtttccaaagaagatttctgacctggaccattgtgccaacagagttctgatgtatggatctgaactagatgcagaccatcctggcttcaaagacaatgtctaccgtaaacgtcgaaagtattttgcggacttggctatgaactataaacatggagaccccattccaaaggttgaattcactgaagaggagattaagacctggggaaccgtattccaagagctcaacaaactctacccaacccatgcttgcagagagtatctcaaaaacttacctttgctttctaaatattgtggatatcgggaggataatatcccacaattggaagatgtctccaactttttaaaagagcgtacaggtttttccatccgtcctgtggctggttacttatcaccaagagatttcttatcaggtttagcctttcgagtttttcactgcactcaatatgtgagacacagttcagatcccttctataccccagagccagatacctgccatgaactcttaggtcatgtcccgcttttggctgaacctagttttgcccaattctcccaagaaattggcttggcttctcttggcgcttcagaggaggctgttcaaaaactggcaacgtgctactttttcactgtggagtttggtctatgtaaacaagatggacagctaagagtctttggtgctggcttactttcttctatcagtgaactcaaacatgcactttctggacatgccaaagtaaagccctttgatcccaagattacctgcaaacaggaatgtcttatcacaacttttcaagatgtctactttgtatctgaaagttttgaagatgcaaaggagaagatgagagaatttaccaaaacaattaagcgtccatttggagtgaagtataatccatatacacggagtattcagatcctgaaagacaccaagagcataaccagtgccatgaatgagctgcagcatgatctcgatgttgtcagtgatgcccttgctaaggtcagcaggaagccgagtatctaacagtagccagtcatccaggaacatttgagcatcaattcggaggtctgggccatctcttgctttccttgaacacctgatcctggagggacagcatcttctggccaaacaatattatcgaattccactacttaaggaatcactagtctttgaaaatttgtacctggatattctatttaccacttatttttttgtttagttttatttctttttttttttggtagcagctttaatgagacaatttatataccatacaagccactgaccacccatttttaatagagaagttgtttgacccaatagatagatctaatctcagcctaactctattttccccaatcctccttgagtaaaatgaccctttaggatcgcttagaataacttgaggagtattatggcgctgactcatattgttacctaagatccccttatttctaaagtatctgttacttattgc

SEQ ID NO: 45 - DNA, 1305 bp, Homo sapiens
  source 1..1305: mol_type = other DNA
ggccacccgcagaacagagcttccgggacccacgcctcgtttgcactgggtgctggacagccgacgcaactacaaatggggcggagctttcggcactggagcagctaatttgcatataggaatgaggtgcggctcggcttccatgggcctaatttacagatagggcggtatttctgccccttaaccgaaagtgggatacagaggacgacggtgttaggcgcctgtgtaggagtaaaatgtgtttattttgcattcaacgagagctcctgcattgcagctattttgcatatgatttgcatcttacgaagaatttgtggcaaaaaaaagctgggcgtgcgccgtaggaacctcctgctgagacgcttccggtagcggcgcgtgacccgacaggtctttcacctacctacctcagctcccacaaacacgagaagttccagcaagttcgccacttccggttctcctggctatccaatagcatcgagaggagcatccccggaagtgaggcagcggaggacgacctttttccggttccggcctggcgagagtttgtgcggcgacatgaaactgcttacccacaatctgctgagctcgcatgtgcggggggtggggtcccgtggcttccccctgcgcctccaggccaccgaggtccgtatctgccctgtggaattcaaccccaacttcgtggcgcgtatgatacctaaagtggagtggtcggcgttcctggaggcggccgataacttgcgtctgatccaggtgccgaaagggccggttgagggatatgaggagaatgaggagtttctgaggaccatgcaccacctgctgctggaggtggaagtgatagagggcaccctgcagtgcccggaatctggacgtatgttccccatcagccgcgggatccccaacatgctgctgagtgaagaggaaactgagagttgattgtgccaggcgccagtttttcttgttatgactgtgtatttttgttgatctataccctgtttccgaattctgccgtgtgtatccccaacccttgacccaatgacaccaaacacagtgtttttgagctcggtattatatatttttttctcattaaaggtttaaaaccaaaagcggtttctctttgcagcaaatatacattaaaatagagtctctgtacagccaagggctctgggccctggcttgccccatgtccctgcgcctccctggccaaacccaaaaataaatatagtgttattgctctgcagggcatagaggcagtgctctcctaccccctgaggaggctcgttgggagctgatggggaagccctg

SEQ ID NO: 46 - DNA, 2770 bp, Homo sapiens
  source 1..2770: mol_type = other DNA
cacacacacacatacacagaatcctcagataacaggaggcaataaatccaacagcacatccacgttcagagaacagtgtccctgctgtcttgctaacagctgccaatacctcactgagtgcctcacaccaacatgggctccaagtgagtttccttcgtctgggcagactccctcccctcttccataaaggctgcaggagacctgtagctgtcacaggaccttccctaagagcccgcaggggaagactgccccagtccggccatcaccatgctccggaccattctggatgctccccagcggttgctgaaggaggggagagcgtcccggcagctggtgctggtggtggtattcgtcgctttgctcctggacaacatgctgtttactgtggtggtgccaattgtgcccaccttcctatatgacatggagttcaaagaagtcaactcttctctgcacctcggccatgccggaagttccccacatgccctcgcctctcctgccttttccaccatcttctccttcttcaacaacaacaccgtggctgttgaagaaagcgtacctagtggaatagcatggatgaatgacactgccagcaccatcccacctccagccactgaagccatctcagctcataaaaacaactgcttgcaaggcacaggtttcttggaggaagagattacccgggtcggggttctgtttgcttcaaaggctgtgatgcaacttctggtcaacccattcgtgggccctctcaccaacaggattggatatcatatccccatgtttgctggctttgttatcatgtttctctccacagttatgtttgctttttctgggacctatactctactctttgtggcccgaacccttcaaggcattggatcttcattttcatctgttgcaggtcttggaatgctggccagtgtctacactgatgaccatgagagaggacgagccatgggaactgctctggggggcctggccttggggttgctggtgggagctccctttggaagtgtaatgtacgagtttgttgggaagtctgcacccttcctcatcctggccttcctggcactactggatggagcactccagctttgcatcctacagccttccaaagtctctcctgagagtgccaaggggactcccctctttatgcttctcaaagacccttacatcctggtggctgcagggtccatctgctttgccaacatgggggtggccatcctggagcccacactgcccatctggatgatgcagaccatgtgctcccccaagtggcagctgggtctagctttcttgcctgccagtgtgtcctacctcattggcaccaacctctttggtgtgttggccaacaagatgggtcggtggctgtgttccctaatcgggatgctggtagtaggtaccagcttgctctgtgttcctctggctcacaatatttttggtctcattggccccaatgcagggcttggccttgccataggcatggtggattcttctatgatgcccatcatggggcacctggtggatctacgccacacctcggtgtatgggagtgtctacgccatcgctgatgtggctttttgcatgggctttgctataggtccatccaccggtggtgccattgtaaaggccatcggttttccctggctcatggtcatcactggggtcatcaacatcgtctatgctccactctgctactacctgcggagccccccggcaaaggaagagaagcttgctattctgagtcaggactgccccatggagacccggatgtatgcaacccagaagcccacgaaggaatttcctctgggggaggacagtgatgaggagcctgaccatgaggagtagcagcagaaggtgctccttgaattcatgatgcctcagtgaccacctctttccctgggaccagatcaccatggctgagcccacggctcagtgggcttcacatacctctgcctgggaatcttctttcctcccctcccatggacactgtccctgatactcttctcacctgtgtaacttgtagctcttcctctatgccttggtgccgcagtggcccatcttttatgggaagacagagtgatgcaccttcccgctgctgtgaggttgattaaacttgagctgtgacgggttctgcaaggggtgactcattgcatagaggtggtagtgagtaatgtgcccctgaaaccagtggggtgactgacaagcctctttaatctgttgcctgattttctctggcatagtcccaacagatcggaagagtgttaccctcttttcctcaacgtgttctttcccgggttttcccagccgagttgagaaaatgttctcagcattgtcttgctgccaaatgccagcttgaagagttttgttttgttttttttcatttatttttttttttaataaagtgagtgatttttctgtggctaaatctagagctgctaaaagggctttaccctcagtgaaaagtgtcttctattttcattatctttcagaaacaggagcccatttctcttctgctggagttattgacattctcctgacctcccctgtgtgttcctaccttttctgaacctcttagactcttagaaataaaagtagaagaaagacagaaaaaataactgattagacccaagatttcatgggaagaagttaaaagaaactgccttgaaatccctcctgattgtagatttcctaacaggaggggtgtaatgtgacattgttcatacttgctaataaatacattattgcctaattcaaaaaaaaaaaaaaaaaa

SEQ ID NO: 47 - DNA, 3885 bp, Homo sapiens
  source 1..3885: mol_type = other DNA
agagccggacggggtaaactgagcggcggcggcggggcgctggggcggagactgcgacccggagccgcccggactgacggagcccactgcggtgcgggcgttggcgcgggcacggaggacccgggcaggcatcgcaagcgaccccgagcggagccccggagccatggccctgagcgagctggcgctggtccgctggctgcaggagagccgccgctcgcggaagctcatcctgttcatcgtgttcctggcgctgctgctggacaacatgctgctcactgtcgtggtccccatcatcccaagttatctgtacagcattaagcatgagaagaatgctacagaaatccagacggccaggccagtgcacactgcctccatctcagacagcttccagagcatcttctcctattatgataactcgactatggtcaccgggaatgctaccagagacctgacacttcatcagaccgccacacagcacatggtgaccaacgcgtccgctgttccttccgactgtcccagtgaagacaaagacctcctgaatgaaaacgtgcaagttggtctgttgtttgcctcgaaagccaccgtccagctcatcaccaaccctttcataggactactgaccaacagaattggctatccaattcccatatttgcgggattctgcatcatgtttgtctcaacaattatgtttgccttctccagcagctatgccttcctgctgattgccaggtcgctgcagggcatcggctcgtcctgctcctctgtggctgggatgggcatgcttgccagtgtctacacagatgatgaagagagaggcaacgtcatgggaatcgccttgggaggcctggccatgggggtcttagtgggcccccccttcgggagtgtgctctatgagtttgtggggaagacggctccgttcctggtgctggccgccctggtactcttggatggagctattcagctctttgtgctccagccgtcccgggtgcagccagagagtcagaaggggacacccctaaccacgctgctgaaggacccgtacatcctcattgctgcaggctccatctgctttgcaaacatgggcatcgccatgctggagccagccctgcccatctggatgatggagaccatgtgttcccgaaagtggcagctgggcgttgccttcttgccagctagtatctcttatctcattggaaccaatatttttgggatacttgcacacaaaatggggaggtggctttgtgctcttctgggaatgataattgttggagtcagcattttatgtattccatttgcaaaaaacatttatggactcatagctccgaactttggagttggttttgcaattggaatggtggattcgtcaatgatgcctatcatgggctacctcgtagacctgcggcacgtgtccgtctatgggagtgtgtacgccattgcggatgtggcattttgtatggggtatgctataggtccttctgctggtggtgctattgcaaaggcaattggatttccatggctcatgacaattattgggataattgatattctttttgcccctctctgcttttttcttcgaagtccacctgccaaagaagaaaaaatggctattctcatggatcacaactgccctattaaaacaaaaatgtacactcagaataatatccagtcatatccgataggtgaagatgaagaatctgaaagtgactgagatgagatcctcaaaaatcatcaaagtgtttaattgtataaaacagtgtttccagtgacacaactcatccagaactgtcttagtcataccatccatccctggtgaaagagtaaaaccaaaggttattatttcctttccatggttatggtcgattgccaacagccttataaagaaaaagaagcttttctaggggtttgtataaatagtgttgaaactttattttatgtatttaattttattaaatatcatacaatatattttgatgaaataggtattgtgtaaatctataaatatttgaatccaaaccaaatataattttttaacttacattaacaaacatttgggcaaaaatcatattggtaatgagtgtttaaaattaaagcacacattatctctgagactcttccaacaaagagaaactagaatgaagtctgaaaaacagaatcaagtaagacagcatgttatatagtgacactgaatgttatttaacttgtagttactatcaatatatttatgcgttaaacagctagttctctcaagtgtagaggacaagaacttgtgtcagttatcttttgaatccataaatcttagctggcattagttttctatgtaatcacctacctagagagagttgtaaattatatgttaacatgttatctggttggcagcaaacactaaagccaataaaggaaaaacagtaaatgttccgaaagcagagaaaagcaaccaaacatattgttatgaactaaaagctttccctttaagatgcatacttgtcttactggatgaagaaaattgagggtacatgtaccttatactgtcaaggttgtttaaacatgataaggttaatcgccatctacttcaagttttagaaaaggaaacaagaagctgaaaacagctgctctgactttaatatctgactatatctttgatctgtttgcaggtcatccaagtgttttctaggaatatatttattttaggttgtctgaaactactattttttagactcctgaaagttgttcacatcaatgtgaagacaaattttaaatgaaaatgaagaatgaaattatgtcttgaatcatatattaagaagtaaaaataatagtgatcaggcagaaaagaaaaatggaacatctaaaaatgtatgtgctaactatatcatccagtgtgcagtgttgtgtatttttctaagcatgacaacattgatgtgccttttcagtgtaacagcaaatactgttagtgaacattgtcaatttatgtcattttgttaagagatatgactggagtgtgcagtgtggaatgtctctaatactacttgtgaatcctgcagttctataatcataaacaaaaattacttagtttcgttaagctaagattgtgtttgtgttaacttcgacatcaaggagcaaagaactttagaacagactcctcaatcttgtgactttcttattctctaggaaagtaacacttcgtttcatgaagcttttctgtggggcttcgattatttcaagtctggtttctaagtgcagtgtgtttgaagcaaacgaacttccaactcacttatttggcattgggcaacttggccaagtctgccactttggaagatggctctggaggaaactctcatatggctaaaaaggcaggctagtttcttacttctacaggggtagagccttaaaaaagaacgtgctacaaattggttctctttgagggtttctggttctccctgcccccaataccatatactttattgcaattttatttttgcctttacggctctgtgtctttctgcaagaaggcctggcaaaggtatgcctgctgttggtccctcgggataagataaaatataaataaaaccttcagaactgttttggagcaaaagatagcttgtacttggggaaaaaaattctaagttcttttatatgactaatattcttggttagcaagactggaaagaggtgtttttttaaaatgtacataccagaacaaagaacatacagctctctgaacatttattttttgaacagaggtggtttttatgtttggacctggtaatacagatacaaaaactttaatgaggtagcaatgaatattcaactgtttgactgctaagtgtatctgtccatattttagcaagtttacttaataaatcttctgaaccatgaaaaaaaaaaaaa

SEQ ID NO: 48 - DNA, 14607 bp, Homo sapiens
  source 1..14607: mol_type = other DNA
ccggaggggctgtcatttgcagcgctggtcgcagccctcagctgcgccgggcggttccggctcctccctctccttgtgcctcagcgccaccatggtgctggagtcggtggtcgcggacttgctgaaccgcttcctgggggactatgtggagaacctgaacaagtcccagctgaagctgggcatctggggcggaaatgtggctttagataatctacagataaaagaaaatgccctgagtgaattggatgttccttttaaagtcaaggctggccaaattgataaattaactttgaagattccttggaagaacctttatggagaagcagttgttgcgaccctggaaggattatacctgcttgttgtccctggagcaagtattaagtatgatgctgtaaaagaagaaaaatccttgcaggatgttaaacagaaagagctatcccgaattgaagaagcccttcaaaaagcagcagaaaaaggcacacattcaggggagttcatatatggcttggagaactttgtttacaaggacatcaagcctggacgtaaacgtaaaaagcacaaaaaacattttaagaaaccttttaaaggtcttgatcgttcaaaagataagccaaaagaagccaaaaaggatacatttgtggaaaaattggcaactcaagtaataaaaaatgtacaagtaaaaatcacagatattcacattaaatatgaagatgatgtcactgatccaaagcggcctctttcatttggtgtcacactgggagagcttagtctactgactgcaaatgaacactggactccatgcatattaaatgaagcagacaaaattatatacaagcttatacgacttgatagtcttagcgcctactggaatgtaaattgcagcatgtcttaccagagatcaagggaacagattttggatcagctgaaaaatgaaattcttacaagtggaaatatacccccaaattatcaatacattttccagccaatatcagcctctgcaaaactctacatgaatccttatgcagaatcagagctcaaaacgcccaaactggattgcaacatagaaatacaaaatattgccattgaactgaccaaacctcagtacttaagtatgattgaccttttggagtcagtggattatatggttaggaatgcgccttataggaaatacaagccttatttaccacttcataccaatggtcgacgatggtggaaatatgcaattgattctgttcttgaagttcatataagaaggtatacacagatgtggtcatggagtaacataaaaaagcacaggcagttactcaagagttataaaattgcctacaaaaacaagttaacacagtctaaagtctcagaagaaatacagaaagaaattcaggacttggagaagactctagatgtttttaacataattttagcaaggcaacaagcacaagttgaggtgattcggtctgggcaaaaattaaggaaaaagtctgctgacacaggcgagaaacgtggaggctggtttagtgggttgtggggtaagaaagagtctaagaaaaaggacgaagaatcattgattcctgaaactattgatgaccttatgactccagaggaaaaagataaactcttcactgccattggttatagtgagagtacccacaacctaactttacctaagcagtatgttgcccatattatgaccctgaagttagtaagcacctctgttacgataagagaaaacaagaatattccagaaatactaaaaattcagataattggcctgggcactcaagtatctcagcgaccaggagcacaagcacttaaggtagaagcgaaattagaacactggtatataacaggtttgagacagcaggatattgtgccatcacttgtggcttcaattggtgacactacatcatccttgcttaaaattaaatttgaaaccaatccggaggatagtcctgctgaccagactctgattgttcagtcccagcctgtggaggtcatctatgatgctaaaactgtcaatgcagtggttgaattctttcaatcaaataagggattggatcttgagcaaataacatcagcaacattgatgaagctggaagaaattaaggagagaacagctacaggacttacacatattattgaaactcgaaaagtccttgatttaaggataaatctgaagccttcttatctagtagttccacagacgggtttccaccatgaaaagtcagatcttctgattttagattttggtacatttcagctcaacagtaaagatcaaggtttacagaagactactaattcatctctggaagaaataatggataaggcatatgacaagtttgatgttgaaataaaaaatgtacaactactttttgcaagagcagaggaaacctggaaaaagtgtcgatttcagcatccatcaactatgcatatattgcaacccatggatattcatgttgagttggctaaggccatggtagaaaaagacattagaatggccagatttaaagtgtcaggaggacttcctttgatgcatgtgagaatttctgaccagaagatgaaagatgtgctatatttgatgaacagtatacctttgccacagaaatcatcagcccagtctccagagagacaggtatcctcaattcctattatttcaggtggtacaaaaggtctacttggtacttcactattgctagacactgtggaatcagagtctgatgatgagtattttgatgctgaagatggagaaccacagacttgtaaaagtatgaaaggatcagaacttaaaaaagctgcagaggtcccaaatgaggagctcatcaatcttctactcaagtttgaaattaaagaagtgattttggaatttactaaacagcagaaagaagaagatacaattctagtatttaatgttactcagttaggaacagaggccacaatgagaacatttgacttaactgtggtatcttatttaaagaaaatcagcttggattatcatgaaattgaaggatccaaaaggaagccccttcacttgattagctcttctgacaaacctggattagatcttttgaaagtggagtatattaaggctgataagaatggacctagttttcaaactgcttttggaaaaactgaacaaacagttaaggtggccttttcatctttaaatctgttgctgcaaacacaagctcttgtcgcttctattaattacctcacaaccattattccatctgatgatcaaagcataagtgttgctaaggaggtacaaatttcaactgaaaaacaacaaaaaaattcaactctgccaaaagcgattgtatcctccagagatagtgacattattgatttcaggctatttgccaagttgaatgctttctgtgtcattgtttgcaacgaaaagaacaatatcgccgaaatcaagattcaaggactggattcctccctttctctccagtcaagaaagcagtcactttttgcccgactagaaaatattattgtcacagatgttgatccaaagacagttcataagaaagctgtgtcaataatgggaaatgaagttttccgttttaatttggatttgtatccagatgctactgagggggatttgtatactgacatgtccaaagtggatggtgtgctgtctctgaatgttggctgtattcagattgtctatcttcataaattccttatgtcacttctgaacttcctgaataatttccagacagccaaagagtctctgagtgctgccactgcccaggctgcagaaagggctgccacaagtgtgaaagatcttgcccagaggagttttcgtgtttccatcaatattgatttgaaagcaccggttatagtcatcccacagtcttctatttccaccaatgcagtagtggtagatcttgggttaatcagagttcataatcagttcagtctggtgtctgatgaagactacttaaatcctccagtaattgatagaatggatgtgcagctaacaaagcttacactttataggacagtgatccagccaggcatctaccatcctgatattcagctgttgcacccaattaacttggaatttcttgtaaatcggaatctagctgcatcttggtaccacaaggtgcctgttgtggaaattaaaggacatcttgattcaatgaatgttagtctaaatcaagaagatcttaatcttttatttaggatactaacagaaaatctctgtgagggtactgaagacttggataaagtgaaaccaagagtacaagagacaggtgaaattaaagagccccttgaaatctctatatcacaagatgtacatgattcaaaaaatactttaacaactggagtggaagaaattaggtctgtagacatcattaatatgctgctgaattttgaaattaaagaggttgtggttactttgatgaaaaaatcagaaaagaaaggaaggcctttacatgagctaaatgtcctgcaacttggaatggaagctaaagttaaaacctatgacatgactgctaaagcttatctaaaaaaaattagtatgcagtgctttgatttcactgactctaaaggggaacctcttcacattattaactcttctaatgtgactgacgaaccccttctgaaaatgttactgacaaaggcagacagtgatggaccagaatttaaaactattcatgacagtaccaaacagagactgaaggtttcatttgcatccttagacttagtacttcatttggaagctttactttccttcatggattttttatcatctgctgctccattctctgagccttcctcttctgagaaggaatccgagctgaaaccacttgtgggggagtccagaagtatcgctgtcaaagctgtatccagcaacatttcccaaaaggatgtgtttgatttaaagatcacagctgaattaaatgcatttaatgtctttgtctgtgatcagaagtgtaacattgcagatattaaaatacatggaatggatgcctctatttctgtgaagcctaagcagactgatgtgtttgccagacttaaagatattatagttatgaatgtagatttgcagtccattcacaaaaaggctgtctctattttgggagatgaagtctttaggttccaactgactctttatccagatgccacagaaggagaggcctatgctgatatgtccaaagtagacggcaaacttagttttaaagtgggttgtattcagattgtttatgttcataaattcttcatgtctcttttgaacttcctcaacaatttccaaactgctaaagaagctttgagtacagccacagtccaggctgcagaaagagctgcttccagcatgaaagacttggctcaaaagagtttccgccttttgatggatattaatttgaaagcaccagttattattattcctcagtcttcagtatcacctaatgctgttatagcagatctgggtttaatcagagttgaaaacaagtttagcttggttcctatggaacattattctcttcctccagtcattgataaaatgaacatcgaactcactcagttgaagctgtcaagaactattttgcaggctagcttgccacaaaatgacattgaaattttaaaaccagtcaacatgcttttgtccatacagcgaaacttagcagcagcatggtatgtgcaaattccagggatggagataaaaggaaaactaaaacctatgcaggttgctctcagtgaagatgacttgacagttttaatgaaaattttgctagaaaatcttggagaagcttcctcacaaccaagccctacacagtctgtgcaggagactgtaagagtgagaaaagttgatgtttcaagtgtacctgaccatctcaaagaacaagaagattggacagactcaaagctctctatgaaccagattgtcagtctccaatttgactttcactttgaatctctttccattatcctttataacaatgatatcaaccaggaatctggagttgcatttcataatgacagtttccaacttggtgaactcagactacatcttatggcctcctcagggaagatgtttaaggatggctcaatgaatgtcagcgttaaacttaagacatgcacccttgatgatctcagagaaggaattgagagagcaacatcgagaatgattgacagaaagaatgaccaagataacaacagttctatgattgatataagttacaaacaagacaaaaatggaagtcaaattgatgctgttcttgacaagctgtatgtatgtgccagtgtggaatttctgatgactgtggcagatttctttatcaaagctgtgcctcagagtccagaaaatgtggcaaaagaaacacagattttaccaagacagactgccacagggaaggtcaagatagagaaagatgactctgttagaccaaatatgactttaaaggccatgatcacagatccagaagtggtatttgttgccagcctgacaaaggctgatgctcctgctctgacagcctcgtttcagtgcaacctttctctgtcaacatccaaactcgaacagatgatggaagcttctgtgagagatctgaaagtgctcgcttgcccttttctcagagaaaagagagggaaaaacattaccacagtcttgcagccctgttctttatttatggaaaaatgtacgtgggcttcaggaaagcaaaatataaatattatggttaaagaatttataattaagatttcacccataattcttaatactgtgttgacaatcatggctgcattgtctccaaaaacaaaagaagatggatccaaagatacgtctaaggaaatggaaaatctttggggtatcaaatcgattaatgattataacacttggtttcttggtgttgacacggcaacagaaataacggaaagcttcaaaggcattgaacattcactgatagaggaaaattgtggtgttgttgtagaatccattcaagttaccttagaatgtggccttggacatcgaactgtacctttattattggcagagtctaagttttcaggaaatattaaaaattggacttctctaatggctgctgttgctgacgtgacactacaggtgcactattacaatgagatccatgctgtctgggagccactgattgagagagtggaggggaagagacaatggaatttaaggcttgatgtaaagaagaacccagttcaggataaaagtttgctgccaggagatgattttattcctgagccacaaatggcaattcatatttcttcaggaaatacaatgaatataacaatatccaaaagttgtcttaatgttttcaacaatttagcaaaaggtttttcagagggcactgcttctacttttgactactctttaaaggacagagctccttttacggtaaaaaatgctgtaggtgttcccattaaggtgaagcccaattgtaatctcagagtaatgggcttccctgagaaaagtgatatttttgatgttgatgctggccagaatttggaactggagtatgccagcatggtaccttcaagtcaagggaacctatctatattgagccgtcaagaaagctccttcttcactctgaccattgtacctcatggatatacagaagttgcaaatatccctgtggccagacctggacggcgattgtataatgtacggaatcccaatgccagtcattctgactctgtcttggtacaaattgatgcaactgaagggaataaagtaattacccttcgctctcctctacagatcaaaaaccatttctccattgcatttatcatctataaatttgttaagaatgttaagctattggagcgcattgggatagccagacctgaagaggagttccatgttcctttagattcatatagatgtcaattgtttatccagccagctggaatcttagagcatcagtacaaagaatctaccacttatatttcctggaaggaagaacttcataggagcagggaagtcagatgcatgttgcagtgtccatcagtagaagtcagcttcttacctctcatagtgaatacagttgctctgcctgatgaattgagctacatatgtacacatggggaagactgggatgtagcttacattattcatctttatccttctctcactttgcggaatcttctcccatattccctaagatatttacttgagggaacagcagaaactcatgagctggcagaaggcagtactgctgatgttctgcattcgagaatcagtggtgaaataatggaattagtcctggtgaaataccagggcaaaaactggaatggacatttccgcatacgtgatacactaccagaattctttcctgtgtgtttttcttctgactccacagaagtgacgacagtcgacctgtcagtccacgtcaggagaattggcagccggatggtgctgtctgtctttagtccctattggttaatcaacaagactacccgggttctccagtatcgttcagaagatattcatgtgaaacatccagctgatttcagggatattattttattttctttcaagaagaagaacatttttactaaaaataaggtacaattaaaaatttcaaccagtgcctggtccagtagtttctcattggatacagtgggaagttatgggtgtgtgaagtgtcctgccaacaatatggagtacctggttggtgttagcatcaaaatgagcagtttcaacctttcacgaatagttaccctgactcccttttgtaccattgcaaacaagtcatcattagaactagaagttggcgagattgcatctgatggctcaatgccaactaataaatggaactatattgcttcttcagagtgccttccattttggccagaaagtttgtcaggcaaactttgtgtgagagtggtgggctgtgaaggatcttccaaaccattcttttataaccgacaggataatggcactttattgagcttagaagatctgaatgggggtatcttggtggatgtaaacactgccgaacattcaactgtcataactttttctgattaccatgagggatctgcacctgccttgataatgaaccatacaccatgggacatcctcacatacaaacagagtgggtcaccagaagaaatggtcttgctgccaagacaggctcgactttttgcctgggcagatcctactggtaccagaaaacttacatggacatatgcagcaaatgttggggaacatgatctgttaaaggatggatgtggacagtttccatatgatgcaaacatccagatacactgggtatcatttctggatgggcgccagagagttttgcttttcaccgatgatgttgccttggtttccaaagcactgcaggcagaagaaatggaacaggctgattatgaaataaccttgtctctccacagtcttgggctttcactggttaacaatgaaagcaagcaggaagtttcctatattgggataaccagttctggtgttgtttgggaggtgaaaccaaagcagaaatggaagccatttagtcaaaagcagataatcttattggaacaatcctatcagaaacatcaaatatcaagagaccatggctggattaagctagataataattttgaggtcaattttgataaagatccaatggaaatgcgcctccctattcgtagccctattaaacgagactttttatcaggaattcagattgaatttaagcagtcttctcaccagagaagtttaagggccaggttgtactggcttcaggttgataatcagttaccaggtgcaatgttccctgttgtatttcatcctgttgcccctccaaaatctattgctttagattcagagcccaagcctttcattgatgtgagtgtcatcacaagatttaatgagtacagtaaagtcttacagttcaagtattttatggtcctcattcaggaaatggccttaaaaattgatcaagggtttctaggagctattattgcactgtttaccccaacaacagaccctgaagctgaaagaagacggacaaagttaatccaacaagatattgatgctctaaatgcagaattaatggagacttcaatgactgatatgtcaattcttagtttctttgaacatttccatatttctcctgtgaagttgcatttgagtttgtctttgggttccggaggtgaagaatcagacaaagaaaaacaggaaatgtttgcagttcattctgtcaacttgctgttgaaaagcataggtgctactctgactgatgtggatgaccttatattcaaacttgcttattatgaaattcgatatcagttctacaagagagatcagcttatatggagtgttgttaggcattacagtgaacagttcttgaaacagatgtatgtccttgtattggggttagatgtacttggaaacccatttggattaattagaggtctgtctgaaggagttgaagctttattctatgaacccttccagggtgctgttcaaggccctgaagaatttgcagaggggttagtgattggagtgagaagcctctttggacacacagtaggtggtgcagcaggagttgtatctcgaatcaccggttctgttgggaaaggtttggcagcaattacaatggacaaggaatatcagcaaaaaagaagagaagagttgagtcgacagcccagagattttggagacagcctggccagaggaggaaagggctttctgcgaggagttgttggtggagtgactggaataataacaaaacctgtggaaggtgccaaaaaggaaggagctgctggattctttaaaggaattggaaaagggcttgtgggtgctgtggcccgtccaactggtggaatcgtagatatggccagtagtaccttccaaggcattcagagggcagcagaatcaactgaggaagtatctagcctccgtccccctcgcctgatccatgaagatggcatcattcgtccttatgacagacaggaatctgagggctctgacttacttgagcaagaactggaaatacaggaataaatgtttcctaaactactacttgatttcatccttaaaaatcaaaacaaactgtggtgttaattgactgtgtgtgaattccattgtcaattttaatgaaattttctttaaaactctcacctccatctgaacttttcatagtagtgggattgactacaaataaaaacttgtggtattcctggtaatactgtccagaaataagagattagtataaaatattaaaggatgcagagaatcagctctcttctgcgtttaatagatgaaagcctttattgagctcagaagcagatactgttactatcatttcgaaaattttatcttatggtgttcatgtgcatttcaggtaaaattgaaaaacaggacaattattatgtccaattaatatgtttatgtttgtgagtcttgatgatggaattacatagctttctgtttcacaaatggctctaaatttgcttaagttacgggactattacctggagcatctgctttaataattgaattgtcagttgctctgagcctgcccttagacctcaagtaataaatagttggcacatgaattttgaggatatgtttcctcttccctctttttcctatttaaccccttggtactgttgctaaataaatgatagccattttataattatgttatatacattttcagcctttagcatttctgcttttcaaaaattgaatctccttgttggttatgcttatttcataattattagttttaattaatgtagatagaagttgaacatgtaattaggcaaattgctgtgtggcacttgaatacatagatttctttattttcaaaaaccaaccttttgcttttaaatccttagagagggtttattatcttagagaaaaaataattataatcattatttttgaaattagtatcctcttaattctcaacataagttatgtttcaatttcttttttttgtaataaatgatggaaatgtttaacaatgtcttatctagcaactttcatgcttctcctcagaaatgaagccaaagtataaacttagatttaatgtgttgtatatttgaagagaatgaaactattaacatataattgttcagttggattatgtattttaaggattgcagttatcaaaataataaattgaatgttttatgtttaaccactttaaagaagaaagactgacatccaaaaaccagcgtgtgctagatatacaaaggaaattacttctgtccttaagggaccaagtataacaaaacatgtaactgttaaaagtagctgacaaacctttcttgtgcctagataatttagcattggcaaaaatgtcaccacatgcagttttctaggagagtcaagcacaaataactaattcaagatgctgacttaaatcatctccaatagttacccttcctgagattctaaagtaacaatttttaattttactggttatattgctgttttactgagacttacttttaagaacccctgtaacttaagattttttcttaattgttttgtttagctctgttattaattttttccttgtgatatctttttataactctctgtcaaaaagcacaaaacttcaagaaacttttaattattttgtctgaacatataatcttgtctgatttcttagtttttattaagatatcagacaacttttaaaactttagtgcattattataattactggaagaaaaagaatgattatacactaatgagaggacttggtagtttttgtcgtggatgtcaagtgtgggcatggataattgaaatatttaggctatttcattctttgcccatcttgctgtgatcagttagttgggtaaaaatatttattgattatttagactgtactggatatacaaaagaagccttctgtccttaagggaccgagtaaaacaaaacatggaaatattaaagagtattagagtataaaagtatatctttttagccctttgtaatatggccaaattctaaataatttatttggggatcttttgatcctcatgttcctttttctcctaagtactactttgtattctttaatatgcagctttgagagttactgaatcatatattatatttccatgagatgtactattctacttatcctctaatcttcatatatatatacacacacacatatatatacacatacatatatacacacgtacatatatgtacacatacagatatacatacacacaaacacatatatacacacatacatatacacacatatatatacacatacaaatatacacatatatacacatacatatatatacacacatacaaatatacccatatgtacacatacatatatacacatacatatatacacacacatatacacacatatatacgcaaacatacacatatttacacatacatatatacatacattatatgtatgtatatatagtcatttaatactcattttggttcacatacttatgatcatgcaacgtttaaaacagcatttcttgctttttagttttagttatatttttccatgttcttagaaatgcctcattaacatttttaattcttgtattgccatctattgaggtgacattacattgtgtttttatctcgtcttaattcatgacattaaattattctactaacagtaataatgctgtaataaacatcattatagattttgcttttttatatcttgtttgctttttcatatttccttagaatttacttgaaaaaattgaattactgggtaaagggcttttgcaaagtattgttaaattcctcgagttgcatttttggaaaggggacgtgaatattttatcaactaatttggtctccctgctgccattagtgactgaatatcttaatctgaatctcagagtgtagtgggtttttagtagtgctgaagacaagttttctaaagtgtattatggtgataaattatattttaaaaactgtcaatggcttgaagcacaatagcctaataactaacgaaaatacatacaagatagaaagtgggtagtatttcttgtacttgcatttcagatctaaatattttaacatatttaaatttcaagctgcagataaatgcattacattattaaattcatttcccattttctctttgaagaaattaaggcaaaagtgttaaaagattttaactaattcgcacaagtgaattgtgaaacaagtagctattgctgtgaaatctgcactcctctctgagactcattctgaagatgagatcccagttctttgtggattcctcttccttattcatggctttttgcaattgtcaaggaatgactaggtaccaagcaactttaaaaaatgtatatttaagcattgaaataatatcaaatgtgatttctctgcttgtggttatattgattatattatccttttaataatattggcattatattcttggtcgtaaaatgtcaaggtcttatttattcagtatatttatgttctgtattttcatatatattatctattttcagccatgcattatatataatgtcagtaatagtatttcattagcattcattataaaaaaactcgtttttaatatttgactaattcaagtcacagtacttttgagatagctgaaaaggaaaataaatgtgttttaatgtgctactaaaaaaaaaaaa

SEQ ID NO: 49 - DNA, 14329 bp, Homo sapiens
  source 1..14329: mol_type = other DNA
gcggccgcagaatcgagctcgggccccggcccccggcccgcggcgcggggctcccgggccccgccgcggacgtcgcgccggtcgccccttccccgtagcccgtgcgccctcggcgcggagccccggcccgccgcggtcccgtctcctgggcctgtcccgcccgcgccctccgccggccctcaggtataatacttctccacgtctgcttcaggaagaaagtgcctgccattcttatcatttctaagcaggttcatgccagcccagaacagagaatcagctggagcccagatttcaagttttgagtaaaataccttcaagcgaatgggccctattgtgctcacacattcagaacctgttacccaaggaattccctaaagaattagaagtgcgtctcaccaaccagccaagatgaacatggtgaagaggatcatggggcggccgaggcaggaggagtgcagcccacaagacaacgccttaggactgatgcacctccgccggctcttcacggagttgtgccatcctccccggcacatgactcagaaggaacaagaagagaaactgtatatgatgctgccagtgtttaacagggtttttggaaatgctccgccgaatacaatgacagaaaaattttctgatcttctgcagttcacaacacaagtctcacgactaatggtgacagaaattcgaaggagagcatcaaacaaatccacagaggctgcaagtcgggccatagttcagttcctagagattaatcagagtgaagaagccagtagaggctggatgcttctaacgacaattaatttgttagcttcctctggtcagaaaaccgtggactgcatgacaacaatgtcagtgccttccaccctggttaaatgtttatatctgttttttgaccttccacatgtgcctgaggcagttggaggtgcacagaatgagctacctctagcagaacgtcgaggactactccagaaagtttttgtacagatcttagtgaaactgtgcagttttgtttcccctgcggaggagctggctcagaaagatgatctccagcttctattcagtgcaataacctcttggtgccctccctataacctgccttggagaaagagtgctggagaagtcctcatgaccatatctcgtcatggtcttagtgtcaatgtagtgaagtatattcatgagaaagagtgtttatctacatgtgttcagaatatgcagcaatcagatgacctgtctcccctagaaattgtcgaaatgtttgctgggctttcttgtttcctcaaagattccagcgatgtttcccaaacacttctggatgattttcggatatggcaaggatataattttctttgtgatctcttgcttagattggaacaagcaaaagaggcagaatccaaagatgccttgaaagatctggttaatctgataacttccctaacaacatatggtgtcagtgaactaaaaccagctggtattaccacaggggcaccctttttattgcctggatttgcagtacctcagcctgcaggcaaaggtcacagtgtgagaaacgtccaggcctttgcagttcttcagaatgcatttttaaaagcaaaaaccagcttccttgcccaaatcatccttgatgctatcacaaatatttacatggctgacaatgccaattacttcatcctagagtcacagcacacattgtcacagtttgcagagaagatttctaaactcccagaagtacaaaacaaatactttgagatgctggagtttgttgtttttagcttaaattatataccttgtaaagaacttattagtgtcagtatcctcttaaaatctagctcttcttatcactgtagcattattgcaatgaaaacacttcttaagtttacaagacatgactacatatttaaagacgtgttcagggaggttggccttttggaggtcatggtaaaccttttgcataaatatgctgccctgttgaaggatccaactcaggcactaaatgaacaaggggactcaagaaataatagttcagttgaagaccaaaaacacctggctttattggttatggagaccttgacagtgcttcttcaaggatcaaacacaaatgcaggaatttttcgagaatttggaggtgcaagatgtgcacataatatagtaaagtaccctcaatgccggcagcatgccttgatgactatccaacagctggtgctctccccaaatggggacgatgacatgggcactctcctggggctaatgcattcagccccaccgacggaattgcagttgaagactgatattttaagggccctcctgtcggtccttcgagaaagccatcgttcaagaacagtttttaggaaagttggaggatttgtgtacattacatccttgctcgttgctatggaaagatctttgagctgtccacccaagaatggctgggagaaagtgaaccagaatcaagtgtttgaacttcttcacactgtgttctgcacgttgactgcagcaatgcgctatgagccagccaactctcatttcttcaaaacagagattcagtatgagaagttggcagatgctgttcgatttcttggctgcttctcagacctaagaaaaataagcgccatgaatgtcttcccctcaaatacacagccatttcaaagacttttagaggaagatgtaatctcaatagaatcagtgtcacccacgttacggcactgcagtaaactttttatttatctttacaaagtagccacagattcttttgacagtcgtgcagaacagatccctccttgcctgacaagtgagtcttctctcccctctccttggggtacaccagctttgtccaggaaaaggcatgcatatcattctgtttcaactccccctgtttaccctcctaaaaatgttgccgacctgaaactacatgtgacaacttcatctctgcagagttctgatgcagtcatcattcatcctggagccatgcttgccatgctggacctactggcctctgttgggtcagtgacacagccagaacatgctttggatcttcaacttgccgtggcaaatattttacaatccctggtgcacacagaaaggaaccagcaagtcatgtgtgaagctggtcttcatgcacgactgctgcagaggtgcagtgctgcattggctgatgaggaccactcactgcacccgcccctgcagcggatgtttgaacgattagcctctcaggctctggaacccatggtgttgagggagtttttacgtttggcaagtcctttaaattgtggtgcctgggacaaaaaactgctaaaacaatatagggtccacaaaccaagttcactgagttatgaaccagaaatgagaagtagtatgatcacatctctggaaggtctgggtactgataatgtttttagcttacatgaagataaccattaccggataagcaagagcctggtaaaatctgcggaaggaagtactgtacccctgaccagggtgaagtgtctggtctccatgacaaccccacatgacatcagacttcatgggtcatcagttactccagcttttgttgaatttgacacatcacttgaagggtttggatgtctttttttgcccagtttggcccctcataatgctcctacaaataataccgtcacaacaggtcttattgatggggctgtggtcagtggcattggttctggtgaaagattcttccctcctccctccggcttaagttactctagctggttttgtattgaacattttagttctcctccaaataaccaccctgtcagacttcttactgttgtgcgccgagcaaattcttctgagcaacattacgtgtgccttgcaatagttctatcagcaaaagaccgatctctgattgtttccaccaaagaggaactcctccaaaattatgttgatgattttagtgaagagtcctcattttatgaaattctcccatgctgtgctcgctttcgatgtggagagcttatcattgagggacagtggcatcatttggtcctggtaatgagcaaaggcatgttgaaaaacagtactgcagccctttatattgatggacagcttgttaacactgtaaagcttcattatgtccacagtactccagggggttcaggttcggcaaatccaccagtggtgagcacggtctatgcctacattggtactccacctgcccaacgccaaattgcctcattggtttggcgcctgggacccacacattttctagaagaagttttaccttcttcaaatgttactaccatttatgaacttggaccaaattatgttggaagctttcaggctgtatgtatgccatgtaaagatgcaaaatccgaaggggtggtgccatcccctgtgtcattagtaccagaggagaaagtgtcatttggcctctatgcactctctgtgtcgtctctaacagtggcaagaatccggaaagtgtataacaaattggatagcaaagccattgctaagcagttaggcatttcctcacatgagaatgccactcctgtgaagttgatacacaattcagcaggacatcttaatggatctgcacggacaattggggccgctctgattggatacttgggagtaagaacatttgtccctaagcctgttgccactactttgcagtacgttggtggagctgcagccatcctgggcctggtggccatggcctctgatgtggaagggttatatgcagcagtcaaggccctggtttgtgtggtcaagagtaacccactagccagcaaagaaatggaaagaatcaagggctaccagttgctggcaatgttgcttaagaagaaacgttcccttcttaacagccacatcctccatctaactttttctttggtgggaactgttgatagtggacatgagacctccattattccaaattcaactgctttccaggacctcctctgtgattttgaagtctggctccatgcaccatatgaacttcatctttccttatttgaacactttattgaactgctcacagagtccagtgaagcctcaaagaatgccaaattaatgagagaattccagttaatcccaaagctgctcctgactcttcgagatatgtctttatcccagcctactattgctgctattagtaatgtcctgagcttcttactgcaaggttttcctagcagcaatgatctgctcagatttgggcagtttatttcttctactttgccaacctttgcggtttgtgagaaatttgtagtaatggaaataaataatgaagagaagcttgacactggaactgaagaggagtttggaggtcttgtatcagctaatcttatacttttgaggaacagacttctggatatcttgctaaaactaatttatacatctaaagaaaagacaagcattaatttgcaagcttgtgaagaactggtgaagacactgggttttgactggatcatgatgtttatggaggaacacttacattccaccacagttacagcagccatgaggattcttgttgtcctactaagtaatcagtctattctcatcaagtttaaagaaggactcagtggtggaggatggcttgaacagacagattctgtcttaactaataagattggaactgtattaggattcaacgtgggcagaagtgctggtgggagatcgacggtcagggagattaaccgagatgcttgtcattttcctggttttccagtccttcagtcattccttcctaaacacactaatgtccctgccctctattttctcctcatggccttgtttctgcagcagccagttagtgagctgcctgagaacctgcaggtcagtgtgcctgtcatcagctgccggagtaagcagggttgccagtttgatttggattccatttggacattcatctttggagttcctgcctccagcggaactgtggtctcttctatccataacgtatgcacagaagctgtttttttattattgggaatgctccgcagcatgctgacttcaccttggcaatcagaagaagagggatcttggctccgagaatatcctgtgaccctgatgcagttcttcagatatttgtatcacaacgtgccagaccttgcctccatgtggatgagccctgacttcctgtgtgcattagcagccaccgtcttccccttcaatattcgcccttactcagagatggtgactgaccttgatgatgaagttggatctccagcagaagagtttaaagcgtttgcagcagacacagggatgaacaggagccaatcagagtactgcaatgtgggcaccaagacatatctgaccaatcacccggctaaaaagttcgtttttgacttcatgcgggtcttaatcatagacaacctctgtctcactcctgccagcaagcaaactccactaattgatcttttgttggaggcttcccctgaaaggtctacaagaactcagcaaaaagaatttcaaacttacattttggatagcgtgatggaccatttgcttgcagctgatgtgttattaggggaagatgcatctctgcctattaccagtggaggaagctaccaggtattggtgaacaatgtgttttatttcacacagcgtgtggtggacaagctttggcaaggcatgttcaacaaagaatctaaacttcttatagattttataattcaactaattgcacagtcaaagagaagatcacagggattgtcactggatgcagtgtatcattgcctcaataggaccatcttgtaccagttctcacgggcacacaaaaccgttcctcagcaagtagctctgcttgattcactcagggtcctcactgtaaacagaaacttgatcctgggacctgggaaccatgaccaagaattcattagctgtctggcccactgcttgataaatctacatgttggaagcaacgtggatggatttggactggaagcagaagcccgcatgaccacatggcacattatgatcccctcggacattgaaccagatggtagttacagccaagatattagtgaagggcgtcagcttctcataaaagctgtcaacagagtttggactgaactgatacatagtaagaaacaagtcttagaggaacttttcaaagtaactctacctgtgaatgaaaggggccacgtggacatagctacagcaaggccactcattgaagaagctgccctgaagtgctggcagaatcatttggcccatgaaaagaaatgcataagtcgaggagaagctttagcgcccaccacacagtccaaattatcccgtgtcagcagtggctttggtctttccaagttaacaggatcaagaaggaatcgaaaagaaagtggtcttaataaacacagtctttccacccaggagatttcgcagtggatgtttactcacattgctgttgttcgtgacttagtagatacacaatataaagaatatcaggagcgtcagcagaatgccctgaagtacgtgacagaagagtggtgtcagatcgagtgcgagctgttgagggagcgggggctgtggggccctcccatcggctcccacctcgacaagtggatgctggagatgacagaagggccctgcaggatgaggaaaaagatggtgcgaaatgatatgttttataaccattacccttacgtgccagaaactgagcaagagacaaatgtggcgtctgagatcccaagtaaacagcctgagacacccgatgatattcctcaaaagaaacctgctcgatatagaagagccgtaagttatgacagtaaagagtactacatgcgactggcctctggcaatcccgccattgtccaagacgccattgtggagagttcagaaggtgaagctgctcagcaagaaccagagcatggggaagacactattgctaaagtcaaaggtttggtcaagcctcctctaaaacgctcccgatctgcacctgatggaggagatgaggagaaccaggagcagctacaagaccagattgctgagggcagctccatagaagaggaggagaaaacagataatgctaccttactgcgcctgttagaggaaggagaaaagatccaacacatgtaccgctgtgctcgagtccagggcctagataccagtgaggggctccttctttttggtaaagagcatttttatgtgattgatggatttaccatgacagcaaccagggaaataagagatattgaaaccttacctccaaatatgcatgagcctattattcctagaggagccaggcaaggccctagtcaactcaagagaacatgcagcatttttgcatatgaagatatcaaggaagttcataaaaggagatatctcctgcagcctattgctgtggaagttttctctggagatggacggaattacctccttgcttttcagaaaggaatcagaaacaaagtctatcaaaggtttttggctgtagtgccatctctaacggacagttcagaatctgtatctgggcaacgaccaaacacgagtgtggagcagggatctgggttacttagcactttggttggagagaagtctgtgactcagagatgggagagaggtgaaatcagcaacttccaatatttgatgcatttgaacactttggctggcagatcatataatgatctcatgcagtatcctgtcttcccctggatccttgcagattatgactcagaggaggtggatcttactaatcccaagacgtttagaaacctggctaagccaatgggagcacaaacagatgaacgattagctcagtataagaagcggtataaagactgggaggatcctaatggagaaactcctgcataccactatgggacccactattcatctgcaatgattgtggcctcataccttgtaaggatggagcctttcacacagatattcttaaggctacagggtggccactttgacctggctgaccggatgtttcacagtgtgcgcgaggcctggtattcagcgtcaaagcacaatatggcagatgtaaaagaacttatcccagagttcttttatttaccagaattcctgttcaattccaacaactttgatctaggctgtaaacaaaatggcaccaagcttggagatgttatccttccaccctgggcaaaaggggacccacgagaattcatcagagtccatcgtgaggctttggagtgtgattacgtgagtgcccatctacatgagtggattgacttaatcttcggttataaacagcaaggccctgctgcagtagaagctgtaaatgtcttccatcatcttttttatgagggtcaagtggatatctacaacatcaatgacccactaaaggagacagccacaattgggttcattaataacttcggtcagatccctaaacagttatttaaaaaacctcatccaccaaagcgagtgagaagtcgactcaatggagacaatgcaggaatctctgtcctaccaggatctacaagtgacaagatcttttttcatcatctagacaacttgaggccttctctaacacctgtaaaagaactcaaagaacctgtaggacaaatcgtatgtacagataaaggtattcttgcggtggaacagaataaggttcttatcccaccaacctggaataaaacttttgcttggggctatgcagacctcagttgcagactgggaacctatgagtcagacaaggccatgactgtttatgaatgcttgtctgagtggggccagattctctgtgcaatctgccccaaccccaagctggtcatcacgggtggaacaagcacggttgtgtgtgtgtgggagatgggcacctccaaagaaaaggccaagaccgtcaccctcaaacaggccttactgggccacactgataccgtcacctgcgccacagcatcattagcctatcacataattgtcagtgggtcccgtgatcgaacctgtatcatttgggatttgaacaaactgtcatttctaacccagcttcgagggcatcgagctccagtttctgctctttgtatcaatgaattaacaggggacattgtgtcctgcgctggcacatatatccatgtgtggagcatcaatgggaaccctatcgtgagtgtcaacacgttcacaggtaggagccagcagatcatctgctgctgcatgtcggagatgaacgaatgggacacgcagaacgtcatagtgacaggacactcagatggagtggttcggttttggagaatggaatttttgcaagttcctgaaacaccagctcctgagcctgctgaagtcctagaaatgcaggaagactgtccagaagcacaaatagggcaggaagcccaagacgaggacagcagtgattcagaagcagatgagcagagcatcagccaggaccctaaggacactccaagccaacccagcagcaccagccacaggccccgggcagcctcctgccgcgcaacagccgcctggtgtactgacagtggctctgacgactccagacgctggtccgaccagctcagtctagatgagaaagacggcttcatatttgtgaactattcagagggccagaccagagcccatctgcagggcccccttagccacccccaccccaatcccattgaggtgcggaattacagcagattgaaacctgggtaccgatgggaacggcagctggtgttcaggagtaagctgactatgcacacagcctttgatcgaaaggacaatgcacacccagctgaggtcactgccttgggcatctccaaggatcacagtaggatcctcgttggtgacagtcgaggccgagttttcagctggtctgtgagtgaccagccaggccgttctgctgctgatcactgggtgaaggatgaaggtggtgacagctgctcaggctgctcggtgaggttttcactcacagaaagacgacaccattgcaggaactgtggtcagctcttctgccagaagtgcagtcgctttcaatctgaaatcaaacgcttgaaaatctcatccccggtgcgtgtttgtcagaactgttattataacttacagcatgagagaggttcagaagatgggcctcgaaattgttgaagattcaacaagctgagtggagaccatggtctgtagaccccttcccgattctcctgtcccagcttggaaggcattgaaaacagtctccgtttacacatctcttcataccacgtgtttgaagtgttaaaattcaaagggatcattgaataaaacgggtgtagagtacaggaatggggcagacgcgattcaggtgaacagcacaagaagaatatgaggtggttcctaggagcaacactttcgacctccagttctccctgatgacagtagctgtctccaagagaaaaatcctcacttattaactctcttttcttgcatctcatttttatagagctactcatccttatttggaaaaaccaacaacaaaaaaggcttttagaaaatggttgtaaatctgacttctttgcaagtaactatgtatattgtaaatagatataaaaggccttttttctaaataaggacttaactgcctgtaacatgaaacttcaaactaaaccactaactcaatgaactacttatggtttgtctgacatccctcacttaccaattaattataaatatgtttttttaaatccccaaagacattatctgtggtctttttttcctttcaagctcagcctgtgtgcctgatgtcatttctttcaagttgcccacagtatctccacttaaactaggctagtaaccaaaataatgtggaccttctttaggaaacagtgtgggagaataggagtccagccgtaagataaactggaaatatttgggcgtcttgtacctggctacgcaccacctcagtgttgttcctacataaacagggccccttttaaacttgtatgtggactgctgtttggtcaaagaataccttcttagcattgcagaaaggtggtcagatgaccagtgtagtgcaggaaacagccctgtctcaactaatggaaatatatttgcatgtaacccaaaattagcttatcttgcatagaacataataagtatgtgtctttggtgacactaatgttctactatagcttattttcaaacaaggggtaaaaaaaggaaagaaagaagtgtacagaattaacatataaactttgttgtaaaactgaatcatgtcagaactgcttaaaattaacctttaccatttaatgtcatctacctgaaaacagtgagatttatactgtatcaatgtctatttttttgtttttgctatgaatataattacagtattttaatatttagttatttaatttgttctactagttggatacagaacacacaaatccagggggattaaagctggaaggggctaagagattagtttacagagaaaaggcttggtggtgggatttttttaaatgtgtgttatgtacatatatatatatatataatatatattaaaaatgaaacaattaatctagattttaacattttcagaaacttagtgataacattatgaacaattctaaaagccctgtgatttgaaaaatatagaatcattaatggcccaagataggccttcacaccttcacaggtgcgaaaggaaaggccttcacaccctcacagaggcatcatgcaaaggacagcggctttggcttttccaattttccatctttaggccctggtgagaggcacacttatgcactaaaatgcacatatatgcacatgcattcaaaaataggcatttggtacaatggtgatcttgtacctgatgggctgaaaccagcttaagaacaaatttgttcttcctgatatgataactaggtctccaagagaaaatagaaaggctgctttagtgccttacgcttactaaatttaaatctttatttacctgggtttgagcctacagtctatttatgattacatatcaaaattgattaaaacacttccatttctaaaagttcaaatatacttgttaataaaaggattatcggcattaatactttaatttaaagaaaagttgtgttctgttttcctttctgtgtcttactccccccacactctccctcccccatcaccatcttcaattctaataaataatgctgatgttcaacagttgcagaaattgtgctattatgtaactgtgggccttgcccctgtctggccctctagatgatttgtagcagtgttattctacactttttaaaagaagcgtcctccttttgtccatgaatcatgtttaccccatacccagtggcagaggtgttctttaaagacttgaatatatgaatgtgtgtgtgtagttacttaaaggttattcctctttgtaataggaaactatatgggatgaacacttttaaactttccgacacaacttccattactaactttctaacagaacttccataactagaaggtggaaaccaaaaccctcatggtagtatttcctctggcagctggtgctgtgggcaactgttttgttcaatcgggtttcttttctttttgcctctaatgcagaaatcaacagaatcactcacacatacaagtacactcacatacataaactaattatttctctggatatctttctgtgttccatgtaaatttatttaccaacatctattgtcaacatgtacatctaccttagtatggtctgcattctttttctgagagtacctcatagggctcctgcctgatctttgtagtttgttcattcatccatccacctgttcatttgttcatccatgtattctaacatttctatgtagtgtgcaactctaatgtcatgcttttgaagaagagaatagctgcccatagcagccatccgtctggataatagcaaaacactctagataagttattttgcactttcttatgtataaagttggtagaaacttatttttgctttgtatcatttaaatacattttgttttggtaaatgaactgtgtataaaatatttatgccgttaaaactgtttttagaaagtatttttaatttcagcaagtttggttacttgttgcatgactcttaacacagctgactttttgtgtcagtgcaatgtatattttttgtcctgttattaacttgtaagccctagtaatggccaattatttgtacagcaacagaagtaaattgaagatactggctaagactggattgattgtggacttttatactatattgcagaaaccaatatctgtttcttggtggttatgtaaaagacctgaagaattactatctagtgtgcagtctgtgatatctgaatgttcattgtatatttgtctctgatgcaaaaaggtagagtaacacaattacaatacatgattaaatgcaatagtccaggtacttaagtaatttttttttcatttcaaataaatacctatttaccaccaaaagaaagaaaaaaaaaaaaaa

SEQ ID NO: 50 - DNA, 12778 bp, Homo sapiens
  source 1..12778: mol_type = other DNA
cgcggcccgagcgcctcttttcgggattaaaagcgccgccagctcccgccgccgccgccgtcgccagcagcgccgctgcagccgccgccgccggagaagcaaccgctgggcggtgagatccccctagacatgcggctcgggggcgggcagctggtgtcagaggagctgatgaacctgggcgagagcttcatccagaccaacgacccgtcgctgaagctcttccagtgcgccgtctgcaacaagttcacgacggacaacctggacatgctgggcctgcacatgaacgtggagcgcagcctgtcggaggacgagtggaaggcggtgatgggggactcataccagtgcaagctctgccgctacaacacccagctcaaggccaacttccagctgcactgcaagacagacaagcacgtgcagaagtaccagctggtggcccacatcaaggagggcggcaaggccaacgagtggaggctcaagtgtgtggccatcggcaaccccgtgcacctcaagtgcaacgcctgtgactactacaccaacagcctggagaagctgcggctgcacacggtcaactccaggcacgaggccagcctgaagttgtacaagcacctgcagcagcatgagagtggtgtagaaggtgagagctgctactaccactgcgttctgtgcaactactccaccaaggccaagctcaacctcatccagcatgtgcgctccatgaagcaccagcgaagcgagagcctgcgaaagctgcagcggctgcagaagggccttccagaggaggacgaggacctggggcagatcttcaccatccgcaggtgcccctccacggacccagaagaagccattgaagatgttgaaggacccagtgaaacagctgctgatccagaggagcttgctaaggaccaagagggcggagcatcgtccagccaagcagagaaggagctgacagattctcctgcaacctccaaacgcatctccttcccaggtagctcagagtctcccctctcttcgaagcgaccaaaaacagctgaggagatcaaaccggagcagatgtaccagtgtccctactgcaagtacagtaatgccgatgtcaaccggctccgggtgcatgccatgacgcagcactcggtgcaacccatgcttcgctgccccctgtgccaggacatgctcaacaacaagatccacctccagctgcacctcacccacctccacagcgtggcacctgactgcgtggagaagctcattatgacggtgaccacccctgagatggtgatgccaagcagcatgttcctcccagcagctgttccagatcgagatgggaattccaatttggaagaggcaggaaagcagcctgaaacctcagaggatctgggaaagaacatcttgccatccgcaagcacagagcaaagcggagatttgaaaccatcccctgctgacccaggctctgtgagagaagactcaggcttcatctgctggaagaaggggtgcaaccaggttttcaaaacttctgctgcccttcagacgcattttaatgaagtgcatgccaagaggcctcagctgccggtgtcagatcgccatgtgtacaagtaccgctgtaatcagtgtagcctggccttcaagaccattgaaaagttgcagctccattctcagtaccatgtgatcagagctgccaccatgtgctgtctttgtcagcgcagtttccgaactttccaggctctgaagaagcaccttgagacaagccacctggagctgagtgaggctgacatccaacagctttatggtggcctgctggccaatggggacctcctggcaatgggagaccccactctggcagaggaccataccataattgttgaggaagacaaggaggaagagagtgacttggaagataaacagagcccaacgggcagtgactctgggtcagtacaagaagactcgggctcagagccaaagagagctctgcctttcagaaaaggtcccaattttactatggaaaagttcctagacccttctcgcccttacaagtgtaccgtctgcaaggaatctttcactcaaaagaatatcctgctagtacactacaattctgtctcccacctgcataagttaaagagagcccttcaagaatcagcaaccggtcagccagaacccaccagcagcccagacaacaaaccttttaagtgtaacacttgtaatgtggcctacagccagagttccactctggagatccatatgaggtctgtgttacatcaaaccaaggcccgggcagccaagctggaggctgcaagtggcagcagcaatgggactgggaacagcagcagtatttccttgagctcctccacgccaagtcctgtgagcaccagtggcagtaacacctttaccacctccaatccaagcagtgctggcattgctccaagctctaacttactaagccaagtgcccactgagagtgtagggatgccacccctggggaatcctattggtgccaacattgcttccccttcagagcccaaagaggccaatcggaagaaactggcagatatgattgcatccaggcagcagcaacaacagcagcagcaacagcaacaacaacaacaacaacaacaacaacaagcacaaacgctggcccaggcccaggctcaagttcaagctcacctgcagcaggagctgcagcaacaggctgccctgatccagtctcagctgtttaaccccaccctccttcctcacttccccatgacaactgagaccctgctgcaactacagcagcagcagcacctcctcttccctttctacatccccagtgctgagttccagcttaaccccgaggtgagcttgccagtgaccagtggggcactgacactgactgggacaggcccaggcctgctggaagatctgaaggctcaggttcaggtcccacagcagagccatcagcagatcttgccgcagcagcagcagaaccaactctctatagcccagagtcactctgccctccttcagccaagccagcaccccgaaaagaagaacaaattggtcatcaaagaaaaggaaaaagaaagccagagagagagggacagcgccgaggggggagagggcaacaccggtccgaaggaaacactgccagatgccttgaaggccaaagagaagaaagagttggcaccagggggtggttctgagccttccatgctccctccacgcattgcttcagatgccagagggaacgccaccaaggccctgctggagaactttggctttgagttggtcatccagtataatgagaacaagcagaaggtgcagaaaaagaatgggaagactgaccagggagagaacctggaaaagctcgagtgtgactcctgcggcaagttgttttccaacatcttgattttaaagagtcatcaagagcacgttcatcagaattactttcctttcaaacagctcgagaggtttgccaaacagtacagagaccactacgataaactgtacccactgaggccccagaccccagagccaccaccacctccccctccaccccctccacccccacttccggcagcgccgcctcagccggcgtccacaccagccatccccgcatcagccccacccatcacctcacctacaattgcaccggcccagccatcagtgccgctcacccagctctccatgccgatggagctgcccatcttctcgccgctgatgatgcagacgatgccgctgcagaccttgccggctcagctacccccgcagctgggacctgtggagcctctgcctgcggacctggcccaactctaccagcatcagctcaatccaaccctgctccagcagcagaacaagaggcctcgcaccaggatcacagatgatcagctccgagtcttgcggcaatattttgacattaacaactcccccagtgaagagcaaataaaagagatggcagacaagtccgggttgccccagaaagtgatcaagcactggttcaggaacactctcttcaaagagaggcagcgtaacaaggactccccttacaacttcagtaatcctcctatcaccagcctggaggagctcaagattgactcccggcccccttcgccggaacctccaaagcaggagtactggggaagcaagaggtcttcaagaacaaggtttacggactaccagctgagggtcttacaggacttcttcgatgccaatgcttacccaaaggatgatgaatttgagcaactctctaatttactgaaccttccaacccgagtgatagtggtgtggtttcagaatgcccgacagaaggccaggaagaattatgagaatcagggagagggcaaagatggagagcggcgtgagcttacaaatgatagatacattcgaacaagcaacttgaactaccagtgcaaaaaatgtagcctggtgtttcagcgcatctttgatctcatcaagcaccagaagaagctgtgttacaaggatgaggatgaggaggggcaggacgacagccaaaatgaggattccatggatgccatggaaatcctgacgcctaccagctcatcctgcagtaccccgatgccctcacaggcttacagcgccccagcaccatcagccaataatacagcttcctccgctttcttgcagcttacagcggaggctgaggaactggccaccttcaattcaaaaacagaggcaggcgatgagaaaccaaagctggcggaagctcccagtgcacagccaaaccaaacccaagaaaagcaaggacaaccaaagccagagctgcagcagcaagagcagcccgagcagaagaccaacactccccagcagaagctcccccagctggtgtccctgccttcgttgccacagcctcctccacaagcgccccctccacagtgccccttaccccagtcgagccccagtccttcccagctctcccacctgcccctcaagcccctccacacatcaactcctcaacagctcgcaaacctacctcctcagctaatcccctaccagtgtgaccagtgtaagttggcatttccgtcatttgagcactggcaggagcatcagcagctccacttcctgagcgcgcagaaccagttcatccacccccagtttttggacaggtccctggatatgcctttcatgctctttgatcccagtaacccactcctggccagccagctgctctctggggccatacctcagattccagcaagctcagccacttctccttcaactccaacctccacaatgaacactctcaagaggaagctggaggaaaaggccagtgcaagccctggcgaaaacgacagtgggacaggaggagaagagcctcagagagacaagcgtttgagaacaaccatcacaccggaacaactagaaattctctaccagaagtatctactggattccaatccgactcgaaagatgttggatcacattgcacacgaggtgggcttgaagaaacgtgtggtacaagtctggtttcagaacacccgagctcgggaaaggaaaggacagttccgggctgtaggcccagcgcaggcccacaggagatgccctttttgcagagcgctcttcaaagccaagactgctcttgaggctcatatccggtcccgtcactggcatgaagccaagagagctggctacaacctaactctgtctgcgatgctcttagactgtgatgggggactccagatgaaaggagatatttttgacggaactagcttttcccacctacccccaagcagtagtgatggtcagggtgtccccctctcacctgtgagtaaaaccatggaattgtcacccagaactcttctaagcccttcctccattaaggtggaagggattgaagactttgaaagcccctccatgtcctcagttaatctaaactttgaccaaactaagctggacaacgatgactgttcctctgtcaacacagcaatcacagataccacaactggagacgagggcaacgcagataacgacagtgcaacgggaatagcaactgaaaccaaatcctcttctgcacccaacgaagggttgaccaaagcggccatgatggcaatgtctgagtatgaagatcggttgtcatctggtctggtcagcccggccccgagcttttatagcaaggaatatgacaatgaaggtacagtggactacagtgaaacctcaagccttgcagatccctgctccccgagtcctggtgcgagtggatctgcaggcaaatctggtgacagcggagatcggcctgggcagaaacgttttcgcactcaaatgaccaatctgcagctgaaggtcctcaagtcatgctttaatgactacaggacacccactatgctagaatgtgaggtcctgggcaatgacattggactgccaaagagagtcgttcaggtctggttccagaatgcccgggcaaaagaaaagaagtccaagttaagcatggccaagcattttggtataaaccaaacgagttatgagggacccaaaacagagtgcactttgtgtggcatcaagtacagcgctcggctgtctgtacgtgaccatatcttttcccaacagcatatctccaaagttaaagacaccattggaagccagctggacaaggagaaagaatactttgacccagccaccgtacgtcagttgatggctcaacaagagttggaccggattaaaaaggccaacgaggtccttggactggcagctcagcagcaagggatgtttgacaacacccctcttcaggcccttaaccttcctacagcatatccagcgctccagggcattcctcctgtgttgctcccgggcctcaacagcccctccttgccaggctttactccatccaacacagctttaacgtctcctaagccgaacttgatgggtctgcccagcacaactgttccttcccctggcctccccacttctggattaccaaataaaccgtcctcagcgtcgctgagctccccaaccccagcacaagccacgatggcgatgggccctcagcaacccccccagcagcagcagcagcagcagcaaccacaggtgcagcagcctcccccgccgccagcagcccagccgccacccacaccacagctcccactgcaacagcagcagcaacgcaaggacaaagacagtgagaaagtaaaggagaaggaaaaggcacacaaagggaaaggggaacccctgcctgtccccaagaaggagaaaggagaggcccccacggcaactgcagccacgatctcagccccgctgcccaccatggagtatgcggtagaccctgcacagctgcaggccctgcaggccgcgttgacttcggaccccacagcattgctcacaagccagttccttccttactttgtaccaggcttttctccttattatgctccccagatccctggcgccctgcagagcgggtacctgcagcctatgtatggcatggaaggcctgttcccctacagccctgcactgtcgcaggccctgatggggctgtccccaggctccctactgcagcagtaccagcaataccagcagagtctgcaggaggcaattcagcagcagcagcagcggcaactacagcagcagcagcagcaaaaagtgcagcagcagcagcccaaagcaagccaaaccccagtcccccccggggctccttccccagacaaagaccctgccaaagaatcccccaaaccagaagaacagaaaaacaccccccgtgaggtgtcccccctcctgccgaaactccctgaagagccagaagcagaaagcaaaagtgcggactccctctacgaccccttcattgttccaaaggtgcagtacaagttggtctgccgcaagtgccaggcgggcttcagcgacgaggaggcagcgaggagccacctgaagtccctctgcttcttcggccagtctgtggtgaacctgcaagagatggtgcttcacgtccccaccggcggcggcggcggtggcagtggcggcggcggcggcggtggcggcggcggcggcggcggcggctcgtaccactgcctggcgtgcgagagcgcgctctgtggggaggaagctctgagtcaacatctcgagtcggccttgcacaaacacagaacaatcacgagagcagcaagaaacgccaaagagcaccctagtttattacctcactctgcctgcttccccgatcctagcaccgcatctacctcgcagtctgccgctcactcaaacgacagcccccctcccccgtcggccgccgccccctcctccgcttccccccacgcctccaggaagtcttggccgcaagtggtctcccgggcttcggcagcgaagcccccttcttttcctcctctctcctcatcttcaacggttacctcaagttcatgcagcacctcaggggttcagccctcgatgccaacagacgactattcggaggagtctgacacggatctcagccaaaagtccgacggaccggcgagcccggtggagggtcccaaagaccccagctgccccaaggacagtggtctgaccagtgtaggaacggacaccttcagattgtaagctttgaagatgaacaatacaaacaaatgaatttaaatacaaaaattaataacaaaccaatttcaaaaatagactaactgcaattccaaagcttctaaccaaaaaacaaaaaaaaaaaaaaaaagaaaaaaaagaaaaagcgtgggttgttttcccatatacctatctatgccggtgattttacattcttgtctttttcttttcttttaatattaaaaaaaaaaaaaaagccctaaccctgttacattgtgtccttttgaaggtactattggtctgggaaacagaagtccgcagggcctccctaatgtctttggagcttaaaccccttgtatatttgccccttttcaataaacgccccacgctgatagcacagaggagcccggcatgcactgtatgggaaagcagtccaccttgttacagttttaaatttcttgctatcttagcattcagataccaatggcttgctaaaagaaaaaaagaaatgtaatgtctttttattctcaggtcaatcgctcacactttgttttcagaatcattgttttatatattattgttttttcagtttttttttttttttttgttccagaaaagattttttgttttgttaacttaaaaatgggcagaaagtattcaagaaaaacaatgtgaactgctttagctttctggggatttttaaggatagcttttctgctgaagccaatttcaaggggaaaagttaagcactcccactttcaaaaaaaaaaaaaaataataacccacacacacaaagagtgttgaggacttgtagcttaaaaaaaataagttttaaaaactgactttctgtatttatgatagatatgaccatttttggtgttgagtagattgttgcattggaaatgaactgaagcagtatggtagatttaaaaggaaaaaaaaaaaaaaaccttttgtgtacatttagctttttgtatggtccagctgacagctcctcatttgatgttgtcttgttcattcctagcagatgatagattgcaatccgttgattcgcctaagcttttctccccttgtcccttaattccactttctctttcttgtcccttaattccactttctctttccttctcccacctcccgtcctataatctcccacttaaggtagctgccttcatttcttagagggagctgcagaattattttataaaactaaagaaagaatttcaagggattctaggggtcattaggatcctcacagattatttttggttggggagttgaaactttttaaaggcatataattctagttacctgtgtctgttagctttgtgcatttattttttatttatccttcttttggcttttttttctttgtaccccttcttttcctccttgtttggtaggagcttcaaatattctttttttttctatactaaaggatttgtttccatttgtgtaattggctgtgtacttttcttttctaaaaaaagtttttggttagggatttggtttttggttttgtgtttgttttttctttcctctctcagaaaaaaaaatttcatgctttaaataaaatccaaagacacaccctttcactgctgatgcagaaaaaagggaaagggttcttgttacttgagaatttgtttctgatttaaacaaacaagacttagtttaataaaagaaagagaaaaacaaaagattcccaggttgttatgtgcttcttctgcaagcagagaggcaaatgttaatgacaattccatataccaaaagacacattttttacttcaaagttttgtccttgtgttaggcagtctgagcagcgagtgatccagagcgcagccaacaaagcagcagatagcagtgtacagaaagcaaaaaaggaactgtatgtgaggcacttgtttctgttaatatccatattcctgttaacacacaccctttctcatgtaaaaagaaaaataaataaatggtctgaactttgaaaactttgtgctgctaaaacatagattttggagacaaataaatagatgctttgctgtttcactttcatagctaaacatcaacagaaaccatctccccttgcccccaaagtgtgaaatccttcttcccttcgttttcttccttatgtttcaaaagggaactttgaagactgtgaatacaggttccattggtcacctttcgggcttctttccccagtgctgaagccactcatcgactttgcaaaagactggagcattccaagatctgaaaatggattttttttctttttttcttttttagccgggactattttatttttatgaatttgtttttagtttaatgaaatagtagatcctgaaatgttgtacatatttctaactaggctgatgcacagtgcaaattccttttttaattgttttttttaagtagaaatactaaagaaagaataccatctaactattcataccagtatccagttgtagcataaggtgtcaaaagcaagtacgcaaaacatttactgttttaacaagctatttccttttaacaagaaatcttgtatttcttcctgtgtttgagatgaacatttttaaattttaaagttgtacagttttttgttttccattattttatcttgtttgtaactctatgaaatatatatatatatattttttgccatttaactgttgtatgttactctgtgtctgtaccatatagaaaaaaaattgtttttgtttttggttctctatgtgatatcagttaacaatgtaacactagctttacctgtcaaattctgctaggtcttctctgaaaacgttgtttttaaaaatgatattgcttggtaatagtgcaatttctatccttttccctcccccctcaacttttaagttcttttctttataattttgctgccccctccctgatggtttgggtttttgtttttgtttttgtttttttttttcatggagctactatgccatcctccctctgtgaggcagagtgactgtcagtgttttgttatgccatgccttgagctgtgggtgtttggcgacaataaggtggttgaatagattggctgagcacacttccacccacctagtgttctcagaggggttatgtgattgtttcaacctggagtgggttgcacccttaatgctttcctctgcaactaaaccgcccacatatatgttcattgaaaaaagtaagaataattctcagcactaacccagaagtagcaaagcagtcagtgatggtgaacattagaggtcaaacatgagttagatgtttgtgggctgacagccatcgtggctatgaccagtactatttacaaagcatgaattcactacaatgctcaactgtttgtttagctttatctcacttggggaatttattcctgtctgctgcattgtaggtagctgggtaggatatatttccacttgctttttaaattagttcttcacctccattgacactcgttttttggttttctccctatagtgtgggttggtgctagacaccagtctgacccacagaatgggagttatttcatccatctttcctccatccttccaaaaaccacatatctacacaaggaaaaatttaatacatctaggaattttttttttaattacaagctatttaaagagatgaatgtggccaaagttttacacaattgaaaataaagtaaaacagacggcatgtgtttaaacctgagtttatcaggcatggcaggaagttgcaggagagagaggcagtgacccaagccagtgcacttgatgttcatggacatatattttttttaaataataaattaaaacattttaaatagaagcataaattgagttgtttgttggcgctgagatactgcccactgtgaaacaaagctttgactagttttttgtttgtttactttcttcaggggggaggggggcaagtttgggtaggaaagaaagcataaatgaacgtgaccctgaggtgaagaggtatatgaacagcctttgcaatgtacaaaaagaaaaaaaaacaaaaaacaacaaaaaaaatagagcaagtgaaaccaaaaatgatgttcttggtgtttttctataatgtagtcttgttagcttttttgttactgtaacaatgctgatctcgaactgtaccaaaatacatggagactaacaaacagaaccacatggaactttcaaactgaaaaaaaaatttgtcacaaaaactttgttgtcatagttaagttgattgtagatggtaattgaatatactcctttgaaaatatttcatcaagtatgtttcctgctcattgtgatacattaaaaaaaaaatatgagcaaaa

SEQ ID NO: 51 - DNA, 4826 bp, Homo sapiens
  source 1..4826: mol_type = other DNA
gggcgcgggcagctctgcgtccgaagctgctccgacgccgtcgctgggaccaagatggacctcccggcgctgctccccgccccgactgcgcgcggagggcaacatggcggcggccccggcccgctccgccgagccccagcgccgctcggcgcgagccccgcgcgccgccgcctgctactggtgcggggccctgaagatggcgggcccggggcgcggcccggggaggcctccgggccaagcccgccgcccgccgaggacgacagcgacggcgactctttcttggtgctgctggaagtgccgcacggcggcgctgccgccgaggctgccggatcacaggaggccgagcctggctcccgtgtcaacctggcgagccgccccgagcagggccccagcggcccggccgccccccccggccctggcgtagccccggcgggcgccgtcaccatcagcagccaggacctgctggtgcgtctcgaccgcggcgtcctcgcgctgtctgcgccgcccggccccgcaaccgcgggcgccgccgctccccgccgcgcgccccaggcctccggccccagcacgcccggctaccgctgccccgagccgcagtgcgcgctggccttcgccaagaagcaccagctcaaggtgcacctgctcacgcacggcggcggtcagggccggcggcccttcaagtgcccactggagggctgtggttgggccttcacaacgtcctacaagctcaagcggcacctgcagtcgcacgacaagctgcggcccttcggctgtccagtgggcggctgtggcaagaagttcactacggtctataacctcaaggcgcacatgaagggccacgagcaggagagcctgttcaagtgcgaggtgtgcgccgagcgcttccccacgcacgccaagctcagctcccaccagcgcagccacttcgagcccgagcgcccttacaagtgtgactttcccggctgtgagaagacatttatcacagtgagtgccctgttttcccataaccgagcccacttcagggaacaagagctcttttcctgctcctttcctgggtgcagcaagcagtatgataaagcctgtcggctgaaaattcacctgcggagccatacaggtgaaagaccatttatttgtgactctgacagctgtggctggaccttcaccagcatgtccaaacttctaaggcacagaaggaaacatgacgatgaccggaggtttacctgccctgtcgagggctgtgggaaatcattcaccagagcagagcatctgaaaggccacagcataacccacctaggcacaaagccgttcgagtgtcctgtggaaggatgttgcgcgaggttctccgctcgtagcagtctgtacattcactctaagaaacacgtgcaggatgtgggtgctccgaaaagccgttgcccagtttctacctgcaacagactcttcacctccaagcacagcatgaaggcgcacatggtcagacagcacagccggcgccaagatctcttacctcagctagaagctccgagttctcttactcccagcagtgaactcagcagcccaggccaaagtgagctcactaacatggatcttgctgcactcttctctgacacacctgccaatgctagtggttctgcaggtgggtcggatgaggctctgaactccggaatcctgactattgacgtcacttctgtgagctcctctctgggagggaacctccctgctaataatagctccctagggccgatggaacccctggtcctggtggcccacagtgatattcccccaagcctggacagccctctggttctcgggacagcagccacggttctgcagcagggcagcttcagtgtggatgacgtgcagactgtgagtgcaggagcattaggctgtctggtggctctgcccatgaagaacttgagtgacgacccactggctttgacctccaatagtaacttagcagcacatatcaccacaccgacctcttcgagcaccccccgagaaaatgccagtgtcccggaactgctggctccaatcaaggtggagccggactcgccttctcgcccaggagcagttgggcagcaggaaggaagccatgggctgccccagtccacgttgcccagtccagcagagcagcacggtgcccaggacacagagctcagtgcaggcactggcaacttctatttggtatgaagcactctattcagtcaccaccatataggtcacttctctcatactcggtcttgaggatattctggattaatcctttctatgcagacgtttctggtttacaaaaggacgcagccctggactacaagtctggaactgacaagttcttatgaccttgacaaatcaccttaacccatctgagccttaaattctcatttatttcctgcataaggagatttggctaaatgctttctgaggtcctttggagtcctgtggctccatggtaatgtgctcctttccttgaagattgggggttttgtaatgttgagatactttgcctctatgcttgtcagctcatgaccagtcctagaagaggagtcgagacataagccaccttcagaggttcaatggaaactttaaaaccataccaaactcttttttaaaattagaattaacaagaaaaaaaaaaagggtggggtttatgagccttagttcttggaggattataagagtacttccccagttttgaggctggacagttaatatactttatatcaattatacatttaatataatttaatttaaaataatttaaagattcttaggagatagtctgactttcctgacctagatgggaatgatcagatagggattttttttgtggcacaggctaaatttgatggtgacatttatattgttgagaatgttacatcttattttaccacaacttttaaaaaatgttacatcttttgcagtaggatcagttgtgaggcacatagtagctgaggctccatggagccacctttcatttctttcagtcagagaggaggacagtctctgtctctgcatttctggtgtcttgcttgtcggtggcagagccatgcttgccggcatttgcttaggcggccatagtagttgctaagtgtacaggtgactgggcagggatgggaggtggccacaggtcagagacaagtgctcagtcagtccctggtgccaggactgtgtgcctcggtgccttgggaaatggaagctccctggtgcagctgcagctgtgggtggaggtagagaagccagcaagaccttggtcttaaccccgtgttcattttcttgctagctgtgtgacgttgggctacctcgcttctctgagtacaaatggtgtgtggtgaatgggtcccaggtatgctacgagctttgagggctgctctttttctcttcatagcgataagtgttaaactgtctttcttaggaaacgttcacagacttgcaacagctgatgtcctctgagtactgtctgactccctcaggcaagttcctgaattcagtaccatcattattatttttgtgtaagactttgacaaagtatagcccctgccaccagagcagcctgtacagtgggtctctaaggtgggacctgccccgggcctgccatgcacgtgtgtgaaacagcgtgaaaagtgtcgcggtaaggtgaccctgggttacccaggcaaggctcggtgtttgtttcagaaagcagagaagtatgtaattgattttaaaagtttctgtttaaaatatttggctatgttttagactatgaaggaatgaactttgcttctctggataagaaagtcacatacattgttccagctccaagtttgttcggccctcgccacaagtggatgtagcgtttggccctttgtgtgccttgctggtgactctggttttgggagctcggatatgtcccagaagcaggcttatggcacttctgtagctcccttgctacccttcctttgtgtctagataagtgactgacatgcttttctttggtctcaggaaagtgggggctcagcaagaactgattaccgagccattcaactagccaaggaaaaaaagcagagaggagcggggagcaatgcaggtgaggccgtgtgtgctgcagccggacgagcaagggcctgagggttctctgtcactgttactggcagaagaaacacagcaggtgtttctgtgctcttggttttacttttctgttcagaatacccttttatcaactccttagttttatttgaacttaagggaaaaaattagtaacaaaattcccagcatcagtatgaacatattttatttgcctaaacaagctttgtgaaagttaagcgttcaaacaccagtgtcagttacctggaaggctactaaggtaaataagcaaagcaggccagttgtcaggaaagcagagattgtgcctggtgctgaatggccttggggcctgatcttggcatggcagagacctggggactgccactgtccccaggtacgtgtacatggagccaaactgtgtgtcctgtggcattgtcagagttatgttgaaatcttatttgaaaatgttagcaacttacttgcatttttaaagaccaaacaagagctggtaacctatggcctcaagcatctgtccttcctaaaaatggaatagtgggatgtagtgcttaatggaaactgctaaatctttttctaaaaactaacagtggatttttaaaatatattgttttttgtgtatttcatttgtcctttgtatttatctaaaagggttgatatgattttatatcttgctctctattcctaatagtattatgacttcttatttaaaataaataacaattgccggttttctgttaaaaaaaaaaaaa

SEQ ID NO: 52 - DNA, 4344 bp, Homo sapiens
  source 1..4344: mol_type = other DNA
gttggcagagcagttgtcctggatggcggagccttgggttccgggggcctgggacctgcaactctttctacaagatatcaagttattctagtacaaccatataaataaataatacctgaagtctcagtgtaacatggacaattaacagtgatgacagataaatacagacgcatggggatcaaatactaggcaaaacgctttttaaaagtgtatcaggcttttaagaaacactgcaggatcctgtctatcttaatgctgatagagctcagctaaaaatttaggaggttctagtattcttcatggctgaagctgagagagtctgaaaccctgatgcttaagctccattctagatcatagctccaactccttcaggatataaggaaaagagattatatttccacaatgatagatctttggttgtacaggtttcccaatgagtggatcatgatgaccgtattgtagggacttgccatagtatggctgcttcccgatctactcgtgttacaagatcaacagtggggttaaacggcttggatgaatctttttgtggtagaactttaaggaatcgtagcattgcgcatcctgaagaaatctcttctaattctcaagtacgatcaagatcaccaaagaagagaccagagcctgtgccaattcagaaaggaaataataatgggagaaccactgatttaaaacagcagagtacccgagaatcatgggtaagccctaggaaaagaggactttcttcttcagaaaaggataacatagaaaggcaggctatagaaaattgtgagagaaggcaaacagaacctgtttcaccagttttaaaaagaattaagcgttgtcttagatctgaagcaccaaacagttcagaagaagattctcctataaaatcagacaaggagtcagtagaacagaggagtacagtagtggacaatgatgcagattttcaagggactaaacgagcttgtcgatgtcttatactggatgattgtgagaaaagggaaattaaaaaggtgaatgtcagtgaggaagggccacttaattctgcagtagttgaagaaatcacaggctatttggctgtcaatggtgttgatgacagtgattcagctgttataaactgtgatgactgtcagcctgatgggaacactaaacaaaatagcattggttcctatgtgttacaggaaaaatcagtagctgaaaatggggatacggatacccaaacttcaatgttccttgatagtaggaaggaggacagttatatagaccataaggtgccttgcacagattcacaagtgcaggtcaagttggaggaccacaaaatagtaactgcctgcttgcctgtggaacatgttaatcagctgactactgagccagctacagggcccttttctgaaactcagtcatctttaagggattctgaggaggaagtagatgtggtgggagatagcagtgcctcaaaagagcagtgtaaagaaaacaccaataacgaactggacacaagtcttgagagtatgccagcctccggagaacctgaaccatctcctgttctagactgtgtttcagctcaaatgatgtctttatcagaacctcaagaacatcgttatactctgagaacctcaccacgaagggcagcccctaccagaggtagtcccactaaaaacagttctccttacagagaaaatggacaatttgaggagaataatcttagtcctaatgaaacaaatgcaactgttagtgataatgtaagtcaatctcctacaaatcctggtgaaatttctcaaaatgaaaaagggatatgttgtgactctcaaaataatggaagtgaaggagtaagtaaaccaccctcagaggcaagactcaatattggacatttgccatctgccaaagagagtgccagtcagcacattacagaagaggaagatgatgatcctgatgtttattactttgaatcagatcatgtggcactgaaacacaacaaagattatcagagactattacagacgattgctgtactcgaggctcagcgttctcaagcagtccaagaccttgaaagtttaggcaggcaccagagagaagcactgaaaaatcccattggatttgtggaaaaactccagaagaaggctgatattgggcttccatatccacagagagttgttcaattgcctgagatcgtatgggaccaatatacccatagccttgggaattttgaaagagaatttaaaaatcgtaaaagacatactagaagagttaagctagtttttgataaagtaggtttacctgctagaccaaaaagtcctttagatcctaagaaggatggagagtccctttcatattctatgttgcctttgagtgatggtccagaaggctcaagcagtcgtcctcagatgataagaggacgcttgtgtgatgataccaaacctgaaacatttaaccagttgtggactgttgaagaacagaaaaagctggaacagctactcatcaaataccctcctgaagaagtagaatctcgacgctggcagaagatagcagatgaattgggcaacaggacagcaaaacaggttgccagccgagtacagaagtatttcataaagctaactaaagctggcattccagtaccaggcagaacaccaaacttatatatatactccaaaaagtcttcaacaagcagacgacagcaccctcttaataagcatctctttaagccttccactttcatgacttcacatgaaccgccagtgtatatggatgaagatgatgaccgatcttgttttcatagccacatgaacactgctgttgaagatgcatcagatgacgaaagtattcctatcatgtataggaatttacctgaatataaagaactattacagtttaaaaagttaaagaagcagaaacttcagcaaatgcaagctgaaagtggatttgtgcaacatgtgggctttaagtgtgataactgtggcatagaacccatccagggtgttcggtggcattgccaggattgtcctccagaaatgtctttggatttctgtgattcttgttcagactgtctacatgaaacagatattcacaaggaagatcaccaattagaacctatttataggtcagagacattcttagacagagactactgtgtgtctcagggcaccagttacaattaccttgacccaaactactttccagcaaacagatgacatggaagagaacatcatttactagtcctcttcaacacatagcaatggtatcattgttaattatgtgcacagtttggaaagattctctgctttcccagaaatgacactcacagcatgagagcttcctgagtgttctcgtcaagtacagctctgcaccgttgtggctctagatcactgttcagcagctgaacattcctggtgagcaaaggtttccctggtgaatttttcaccactgcgttttaggtggtgatcttaaatgggtgagatggaacgagagcacacattaaagagagagtaaattccaaaggtttcaaagaacttggtcataaatatgataatgagaagacaaagtatttatattaaaacagtttagtagccttcagttttgtgaaaatagttttcagcacagaaactgacttctttagacaaagttttaaccaatgatggtgtttgcttctaggatatacactttaaaagaactcactgtcccagtggtggtcattgatggcctttagtaaattggagctgcttaatcatattgatatctaatttcttttaaccacaatgaattgtccttaattaccaacagtgaagcactacaggaggcaactgtggcattgcttccttaaccagctcatggtgtgtgaatgttataaaattgtcactcagatatattttttaaatgtaatgttatataagatgatcatgtgatgtgtacaaactatggtgaaaagtgccagtggtagtaactgtgtaaagtttctaattcacaacattaattcctttaaaatacacagccttctgcctctgtatttggagttgtcagtacaactcatcaaagaaaactgcctaatataaaaatcatatatatggtaataatttccctcttttgtagtctgcacaagatccataaaagattgtatttttattactatttaaacaagtgattaaatttagtctgcacagtgagcaagggttcacatgcattcttttatactgctggattttgttgtgcatcatttaaaacattttgtatgtttcttcttatctgtgtatacagtatgttcttgaatgatgttcatttgtcaggagaactgtgagaaataaactatgtggatactgtctgtttatattaaaagaaaaaaaaaaaaaaaaa